SEQUENCE LISTING (253 sequences)

SEQ ID NO: 1 - RNA, 1065 nt, Homo sapiens
augcuguuuaaucugaggauccuguuaaacaaugcagcuuuuagaaauggucacaacuucaugguucgaaauuuucgguguggacaaccacuacaaaauaaagugcagcugaagggccgugaccuucucacucuaaaaaacuuuaccggagaagaaauuaaauauaugcuauggcuaucagcagaucugaaauuuaggauaaaacagaaaggagaguauuugccuuuauugcaagggaaguccuuaggcaugauuuuugagaaaagaaguacucgaacaagauugucuacagaaacaggcuuugcacuucugggaggacauccuuguuuucuuaccacacaagauauucauuugggugugaaugaaagucucacggacacggcccguguauugucuagcauggcagaugcaguauuggcucgaguguauaaacaaucagauuuggacacccuggcuaaagaagcauccaucccaauuaucaaugggcugucagauuuguaccauccuauccagauccuggcugauuaccucacgcuccaggaacacuauagcucucugaaaggucuuacccucagcuggaucggggaugggaacaauauccugcacuccaucaugaugagcgcagcgaaauucggaaugcaccuucaggcagcuacuccaaaggguuaugagccggaugcuaguguaaccaaguuggcagagcaguaugccaaagagaaugguaccaagcuguugcugacaaaugauccauuggaagcagcgcauggaggcaauguauuaauuacagacacuuggauaagcaugggacaagaagaggagaagaaaaagcggcuccaggcuuuccaagguuaccagguuacaaugaagacugcuaaaguugcugccucugacuggacauuuuuacacugcuugcccagaaagccagaagaaguggaugaugaagucuuuuauucuccucgaucacuaguguucccagaggcagaaaacagaaaguggacaaucauggcugucauggugucccugcugacagauuacucaccucagcuccagaagccuaaauuuuga

SEQ ID NO: 2 - DNA, 1065 bp, Homo sapiens
atgctgtttaatctgaggatcctgttaaacaatgcagcttttagaaatggtcacaacttcatggttcgaaattttcggtgtggacaaccactacaaaataaagtgcagctgaagggccgtgaccttctcactctaaaaaactttaccggagaagaaattaaatatatgctatggctatcagcagatctgaaatttaggataaaacagaaaggagagtatttgcctttattgcaagggaagtccttaggcatgatttttgagaaaagaagtactcgaacaagattgtctacagaaacaggctttgcacttctgggaggacatccttgttttcttaccacacaagatattcatttgggtgtgaatgaaagtctcacggacacggcccgtgtattgtctagcatggcagatgcagtattggctcgagtgtataaacaatcagatttggacaccctggctaaagaagcatccatcccaattatcaatgggctgtcagatttgtaccatcctatccagatcctggctgattacctcacgctccaggaacactatagctctctgaaaggtcttaccctcagctggatcggggatgggaacaatatcctgcactccatcatgatgagcgcagcgaaattcggaatgcaccttcaggcagctactccaaagggttatgagccggatgctagtgtaaccaagttggcagagcagtatgccaaagagaatggtaccaagctgttgctgacaaatgatccattggaagcagcgcatggaggcaatgtattaattacagacacttggataagcatgggacaagaagaggagaagaaaaagcggctccaggctttccaaggttaccaggttacaatgaagactgctaaagttgctgcctctgactggacatttttacactgcttgcccagaaagccagaagaagtggatgatgaagtcttttattctcctcgatcactagtgttcccagaggcagaaaacagaaagtggacaatcatggctgtcatggtgtccctgctgacagattactcacctcagctccagaagcctaaattttga

SEQ ID NO: 3 - protein, 353 aa, Homo sapiens
MLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIFGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 4 - protein, 355 aa, Artificial Sequence
  Synthetic construct
MLVFNLRILLNNAAFRNGHNFMVRNFRCGQPLQNRVQLKGRDLLTLKNFTGEEIRYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 5 - RNA, 129 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauag

SEQ ID NO: 6 - RNA, 28 nt, Artificial Sequence
  Synthetic construct
auuauuacaucaaaacaaaaagccgcca

SEQ ID NO: 7 - RNA, 245 nt, Artificial Sequence
  Synthetic construct
cuuaagggggcgcugccuacggagguggcagccaucuccuucucggcaucaagcuuaccauggugccccaggcccugcucuuggucccgcugcugguguucccccucugcuucggcaaguuccccaucuacaccauccccgacaagcuggggccguggagccccaucgacauccaccaccuguccugccccaacaaccucguggucgaggacgagggcugcaccaaccugagcggguucuccuac

SEQ ID NO: 8 - RNA, 177 nt, Artificial Sequence
  Synthetic construct
ugagugucguacagccuccaggccccccccucccgggagagccauaguggucugcggaaccggugaguacaccggaauugccgggaagacuggguccuuucuuggauaaacccacucuaugcccggccauuugggcgugcccccgcaagacugcuagccgaguaguguuggguugcg

SEQ ID NO: 9 - RNA, 89 nt, Artificial Sequence
  Synthetic construct
aauuauugguuaaagaaguauauuagugcuaauuucccuccguuuguccuagcuuuucucuucugucaaccccacacgccuuuggcaca

SEQ ID NO: 10 - RNA, 569 nt, Artificial Sequence
  Synthetic construct
cucccuccccccccccuaacguuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacguguguuuagucgagguuaaaaaacgucuaggccccccgaaccacggggacgugguuuuccuuugaaaaacacgaugauaau

SEQ ID NO: 11 - RNA, 44 nt, Artificial Sequence
  Synthetic construct
gucagcuuucaaacucuuuguuucuuguuuguugauugagaaua

SEQ ID NO: 12 - RNA, 54 nt, Artificial Sequence
  Synthetic construct
cucucgccugagaaaaaaaauccacgaaccaauuucucagcaaccagcagcacg

SEQ ID NO: 13 - RNA, 52 nt, Artificial Sequence
  Synthetic construct
accugugaggguucgaaggaaguagcaguguuuuuuguuccuagaggaagag

SEQ ID NO: 14 - RNA, 71 nt, Artificial Sequence
  Synthetic construct
acacagaaacauucgcaaaaacaaaaucccaguaucaaaauucuucucuuuuuuucauauuucgcaaagac

SEQ ID NO: 15 - RNA, 52 nt, Artificial Sequence
  Synthetic construct
cagaaaaauuugcuacauuguuucacaaacuucaaauauuauucauuuauuu

SEQ ID NO: 16 - RNA, 158 nt, Artificial Sequence
  Synthetic construct
cuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacau

SEQ ID NO: 17 - RNA, 166 nt, Artificial Sequence
  Synthetic construct
ugcaaggcuggccggaagcccuugccugaaagcaagauuucagccuggaagagggcaaaguggacgggaguggacaggaguggaugcgauaagaugugguuugaagcugaugggugccagcccugcauugcugagucaaucaauaaagagcuuucuuuugacccau

SEQ ID NO: 18 - RNA, 143 nt, Artificial Sequence
  Synthetic construct
acgccgaagccugcagccaugcgaccccacgccaccccgugccuccugccuccgcgcagccugcagcgggagacccuguccccgccccagccguccuccugggguggacccuaguuuaauaaagauucaccaaguuucacgca

SEQ ID NO: 19 - RNA, 220 nt, Artificial Sequence
  Synthetic construct
uagagcggcaaacccuagcuacacuccauagcuaguuucuuuuuuuuuuguuuuuuuuuuuuuuuuuuuuuuuuuuuuuuuuuuuuuuuccuuucuuuuccuucuuuuuuuccucuuuucuugguggcuccaucuuagcccuagucacggcuagcugugaaagguccgugagccgcaugacugcagagagugccguaacuggucucucugcagaucaugu

SEQ ID NO: 20 - RNA, 170 nt, Artificial Sequence
  Synthetic construct
acacaucacaaccacaaccuucucaggcuacccugagaaaaaaagacaugaagacucaggacucaucuuuucuguugguguaaaaucaacacccuaaggaacacaaauuucuuuaaacauuugacuucuugucucugugcugcaauuaauaaaaaauggaaagaaucuac

SEQ ID NO: 21 - RNA, 110 nt, Artificial Sequence
  Synthetic construct
gcuggagccucgguagccguuccuccugcccgcugggccucccaacgggcccuccuccccuccuugcaccggcccuuccuggucuuugaauaaagucugagugggcagca

SEQ ID NO: 22 - RNA, 123 nt, Artificial Sequence
  Synthetic construct
uagugcagucacuggcacaacgcguugcccgguaagccaaucggguauacacggucgucauacugcagacaggguucuucuacuuugcaagauagucuagaguaguaaaauaaauaguauaag

SEQ ID NO: 23 - RNA, 6 nt, Artificial Sequence
  Synthetic construct
gccacc

SEQ ID NO: 24 - RNA, 4 nt, Artificial Sequence
  Synthetic construct
gcca

SEQ ID NO: 25 - RNA, 9 nt, Artificial Sequence
  Synthetic construct
auaagugaa

SEQ ID NO: 26 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuguuuaaucugaggauccuguuaaacaaugcagcuuuuagaaauggucacaacuucaugguucgaaauuuucgguguggacaaccacuacaaaauaaagugcagcugaagggccgugaccuucucacucuaaaaaacuuuaccggagaagaaauuaaauauaugcuauggcuaucagcagaucugaaauuuaggauaaaacagaaaggagaguauuugccuuuauugcaagggaaguccuuaggcaugauuuuugagaaaagaaguacucgaacaagauugucuacagaaacaggcuuugcacuucugggaggacauccuuguuuucuuaccacacaagauauucauuugggugugaaugaaagucucacggacacggcccguguauugucuagcauggcagaugcaguauuggcucgaguguauaaacaaucagauuuggacacccuggcuaaagaagcauccaucccaauuaucaaugggcugucagauuuguaccauccuauccagauccuggcugauuaccucacgcuccaggaacacuauagcucucugaaaggucuuacccucagcuggaucggggaugggaacaauauccugcacuccaucaugaugagcgcagcgaaauucggaaugcaccuucaggcagcuacuccaaaggguuaugagccggaugcuaguguaaccaaguuggcagagcaguaugccaaagagaaugguaccaagcuguugcugacaaaugauccauuggaagcagcgcauggaggcaauguauuaauuacagacacuuggauaagcaugggacaagaagaggagaagaaaaagcggcuccaggcuuuccaagguuaccagguuacaaugaagacugcuaaaguugcugccucugacuggacauuuuuacacugcuugcccagaaagccagaagaaguggaugaugaagucuuuuauucuccucgaucacuaguguucccagaggcagaaaacagaaaguggacaaucauggcugucauggugucccugcugacagauuacucaccucagcuccagaagccuaaauuuugacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 27 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcucuuuaaucugcgcaucuuacugaacaacgccgcauuccggaacggucacaacuucaugguccgcaauuuccgcuguggccagccgcuucaaaacaagguccagcugaagggacgggaucugcugacacugaagaacuucaccggagaagagaucaaguacaugcuguggcucagcgcagacuugaaguuccggaucaagcagaagggagaauacuugccccugcugcaaggaaagucgcugggaaugauuuuugagaagcggucaacucgcaccagacucuccaccgaaacugguuucgcacugcuuggcgggcacccuugcuuccugacgacucaggacauccaccucggcgugaacgaaucgcuaaccgauaccgccagagugcuuucuuccauggccgacgcggugcuggccaggguguacaagcaguccgaccucgauaccuuggcaaaggaggcuuccauucccaucaucaacggccugagcgaccuguaccacccaauccaaauccuggcugacuaccugacccugcaagagcacuacagcagccugaagggucugacccugucauggauuggcgauggaaacaauauucugcacuccaucaugauguccgccgcgaaguucggaaugcaucugcaagccgccacuccaaaaggauacgaaccggaugcguccgugaccaaguuggcggaacaguacgcgaaggagaacggaaccaagcuucugcugacuaacgacccccucgaggcugcgcaugggggcaacgugcugauuaccgacaccuggaucuccauggggcaggaggaagagaagaagaagagacugcaggcauuccagggguaccaggucaccaugaaaaccgcaaaaguggcagcuucggacuggacuuuccugcauugccugccgaggaagccggaggaagucgacgacgaaguguucuacucgccucggucccugguguuccccgaggccgaaaaccggaaguggaccaucauggccgugaugguguccuugcugacugacuauagcccgcagcugcagaagccuaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 28 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuguuuaaccuacguauuuugcucaacaaugcagccuuuagaaacggacauaacuuuaugguucgaaacuuucgcugcgggcagccacugcagaacaagguccagcugaaagggagagauuugcucacgcugaagaacuuuacuggcgaagaaaucaaguauaugcugugguuguccgcggaccucaaguuucggauuaagcagaaaggggaguaucugccacugcugcaaggaaagagccucggcaugaucuucgagaagcggagcacucggaccaggcugaguaccgaaacuggcuucgcauuguuggguggacauccauguuuucugacaacgcaggacauucaucugggcgugaacgagagucugacggacacagcucgcguucuguccucuauggcugaugcgguguuggcccgggucuauaagcaguccgauuuggacaccuuggcuaaggaagcuagcauaccgauuaucaaugggcuguccgaccuguaucacccuauucaaauccuggccgacuaccucacacugcaagaacacuauagcucauugaagggacugacccugagcuggauaggggacggaaacaacauccuacauagcauuaugauguccgcugccaaguuuggcaugcaucuucaagccgccacgccaaaggguuaugagcccgacgcgucagugacaaagcuggccgagcaguacgcuaaggagaaugguaccaaauuacugcugacuaaugauccacuggaggcugcacauggcggcaauguacugaucaccgacacauggaucucgaugggccaggaggaagaaaagaagaagaggcuucaggccuuccaaggcuaccaggucaccaugaaaacagcuaagguugcagcaucugauuggaccuuucugcacugucugccaaggaagcccgaagagguggacgaugaaguauucuauagcccacggaguuugguguucccugaggcugaaaauaggaaguggacaauuauggccguaauggugucccuguuaaccgacuacucuccgcaacugcagaaaccuaaguuuuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 29 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuguuuaacuuaaggauccugcugaacaacgccgcuuuucguaacggucauaacuuuaugguccggaacuuuagauguggccagccgcugcagaacaagguucagcugaaggggagggaucugcugaccuugaagaacuuuaccggcgaagagaucaaguacauguuguggcugagcgccgaucugaaguuuaggauuaagcagaagggggaguauuugccacugcugcaaggaaaauccuuggggaugaucuucgagaagcgcuccacuagaacccggcuaagcacagaaaccggcuucgcacuucuggguggacaucccuguuuucugacgacgcaggauauacaccugggcgugaaugagagucugacggacacagcuaggguguugagcagcauggccgaugcaguacuggcccgcguuuauaagcagagcgacuuggacacacuggccaaggaagcgucaauuccgauuaucaaugggcugucagaccuguaucaucccauucaaaucuuggcugacuaucugacccugcaagaacauuacagcucccugaagggccucacguuguccuggauuggcgacggaaacaacauucugcauucgaucaugaugagcgcugcuaaguuuggcaugcaccuccaagccgcuacaccuaagggauaugagccugaugccagcguaaccaagcuggccgaacaguacgcgaaggagaauggcacgaaacugcuguugacaaaugacccacuggaggcagcucacgguggcaacgugcugaucaccgacacguggauaucuaugggacaggaagaagagaagaagaagcggcugcaggcauuccaaggguaucaggucaccaugaaaacggccaagguugcugcauccgacuggacauuucugcauugcuugccccgcaaaccagaagaaguagacgacgaagucuuuuauuccccacggucgcugguguuccccgaggcggagaaucgaaaguggacgauuauggccgugauggugucccugcugacugauuacucuccccaacugcaaaagccuaaguuuuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 30 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuuuucaaccugaggauccuccugaacaacgccgccuuucgcaauggucacaacuuuaugguccggaacuucagaugcggccagccgcugcagaacaagguccagcugaagggacgggaucugcugacucugaagaacuucaccggagaagagaucaaguacaugcuguggcugucggccgaccugaaguucaggaucaagcagaagggagaauaccucccgcugcugcaaggaaagucccugggcaugauuuucgagaagcgcucgaccagaacucgguuguccaccgaaaccggguuugcgcugcugggcggacauccuugcuuccugacgacucaggauauucaccugggagugaacgagucgcugaccgacaccgccagagugcugagcucgauggccgacgccguguuggcacgcguguacaagcaguccgaucuggauacccuggccaaagaagcuuccaucccgaucauuaacgggcugagcgaccucuaccaccccauucaaauccuggccgacuaccugacucugcaagaacacuacagcucgcugaagggguugacucuguccuggaucggcgacggaaacaacauccugcacuccaucaugaugucggccgcaaaguucggcaugcauuugcaagccgccaccccaaagggcuacgaaccagacgcgagcgucaccaagcuggccgaacaguacgcgaaggaaaaugguacuaagcugcugcugaccaacgacccauuggaagcugcccaugguggaaacgugcugaucaccgacaccuggaucucgaugggccaggaagaggagaagaagaagcggcugcaggcguuccagggguaucaggucaccaugaaaacagccaaaguggcagcgucagacuggaccuuccuccacugucugccucgcaagccagaggagguggacgacgagguguucuacuccccucggucccucguguucccugaggcugagaaccggaaguggaccauuauggccgugauggugucacuccugacugauuacuccccgcaacugcagaagcccaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 31 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuguuuaaccugaggauccuauugaacaaugcugcuuuucguaauggccauaacuuuaugguucggaacuuuagaugcgggcagccacugcagaacaagguccaguugaaaggccgcgaucuguugacauugaagaacuuuaccggcgaagagauuaaguauaugcuguggcugucugcugaccucaaguuucgaaucaagcagaagggcgaauaucucccccugcugcaaggaaagucucucggcaugaucuuugagaagcggaguacccgaacacggcugagcaccgaaacgggcuucgcacugcuggggggccaucccuguuuucugacaacgcaggacauccacuuggggguuaacgaaucauugacugauaccgcccgcguacugucauccauggccgacgcugugcuggcuaggguguacaagcagucagaucuggauacacuggccaaggaagcuagcauaccaaucaucaauggacugagugaccuuuaucacccgauucaaauacuagccgauuaucugacccugcaagagcauuacuccucgcugaaaggccucacgcuguccuggaucggcgacggcaacaacauucugcauaguauuaugaugucugcugccaaauucggcaugcaucugcaagcugcuacgccgaaggguuaugaacccgacgcgucaguuacgaagcucgcugagcaguaugcaaaggagaauggcacaaagcuguugcuuaccaacgauccccuggaagcugcucauggcggcaaugugcugauuacugacaccuggauuucaaugggccaggaggaggagaagaagaagagguuacaggcuuuucaagguuaccaagucacgaugaaaaccgcuaaggucgcagccagcgacuggacauuccugcacugucugccaagaaagccggaagaaguggacgacgagguguucuauuccccgcggucuuugguguuuccggaggccgaaaacaggaaauggaccauuauggccgugaugguaucguugcugacggacuacagcccucaguugcaaaagcccaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 32 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcucuuuaaccuccgcauccuccucaacaacgccgccuuccggaaugggcauaacuucaugguccggaacuucagaugcggccagccccugcaaaacaagguccaguugaagggacgggaccuccuuacgcugaagaacuuuaccggagaagagauuaaguacaugcugugguuguccgcugaccucaaguuccgcauuaagcagaagggagaauaucugccgcugcugcaaggaaagagccugggcaugaucuucgaaaagcgcuccacuagaacccggcugucgacugagacuggauucgccuugcucgguggacacccgugcuuccugacgacccaggacauccaccugggagugaacgagucacuuacggauaccgcgagggugcuguccucaauggccgacgcagugcucgcgcgcguguacaagcagucagaucuggauacccuggccaaggaagccagcauucccaucaucaacggacugagcgaccuuuaccacccaauccagauccucgccgacuacuuaacccugcaagagcacuacagcucccugaagggacugacucuguccuggaucggggauggaaacaacauccugcacuccaucaugaugucugccgcuaaguuugggaugcaucugcaagccgcaaccccuaagggauacgagcccgacgccucggugaccaaacuugcggaacaguacgccaaggaaaacgguaccaagcugcugcugaccaacgacccucuggaagcggcccacggaggaaaugugcugauuaccgacaccuggauuucgaugggccaggaggaggagaagaagaagagacugcaggcguuccagggauaucaggucaccaugaaaaccgccaaggucgccgccagcgacuggaccuuccugcacugucucccucggaaaccggaagaaguggaugacgagguguucuacuccccgcgcucgcugguguucccggaggcugaaaacaggaaguggacaaucauggccgugauggugucccuguugaccgacuacuccccacaacugcagaagcccaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 33 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuuuucaaucugcgcauccuccugaacaacgccgccuuccgcaauggacacaacuuuaugguccgcaacuuccgcugugggcagccgcugcagaacaagguccagcucaaggggagagaucuccugacccugaagaacuucacuggagaggagaucaaguacaugcuguggcuguccgccgaccugaaauuucggauuaagcagaagggcgaauaccucccacugcugcaaggaaagucuuugggcaugaucuucgaaaagagaagcacccggacccgguugagcaccgaaacuggguucgcgcuccucgguggacacccgugcuuccugaccacccaagauauucaucugggugucaacgaaagccugaccgacaccgccagggugcugucauccauggcugacgcagugcucgcccggguguacaagcagucagaccuggacacccucgccaaggaagcuucgaucccuaucaucaacggacuuuccgaccuguaccaccccauccaaauucuggccgacuaccugacucugcaagaacacuauagcucgcugaaaggacuuacucuguccuggaucggggacggcaacaacauucuccauuccaucaugauguccgcugccaaguucggaaugcaccuucaagcagcgacucccaagggauacgaaccugaugccuccgugacuaagcuggcagagcaguacgccaaggagaacgguacaaagcugcugcucacgaacgacccccuggaggcggcccacggcggaaacgugcugauuaccgauaccuggaucucaaugggccaggaagaggagaagaagaagcggcuccaggcguuucaaggcuaccaggucaccaugaaaaccgcgaaggucgccgccuccgacuggacuuucuugcacugccugccgcggaagcccgaggaaguggaugacgaaguguucuacucgccgagaucguugguguucccugaggccgaaaacaggaaguggaccaucauggccgugauggugucccugcugacugauuacagcccacagcugcagaagccuaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 34 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuuuucaaucuccgcauccuccuuaacaacgccgcguuuagaaacggccacaacuucaugguccggaacuucagauguggccagccgcuucaaaacaagguccagcugaagggccgggaucuucugacccugaagaacuuuacuggcgaagagaucaaguacaugcucuggcucuccgcggacuugaaguuccgcauuaagcagaagggggaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucuggacacucuggccaaggaggcgucaauuccuauuaucaacggccuuagugaccucuaccauccgauucagauccuggccgauuaccucacccugcaagaacacuacagcucccugaagggucugacauuguccuggaucggcgacggcaacaacauucuccauuccaucaugauguccgccgcaaaauucggcaugcaucuucaagccgccacgccgaaggguuacgagcccgacgcuuccgugacuaagcucgccgagcaguacgcuaaggagaacggaaccaagcuucugcugacuaacgacccacuagaagcagcccacgggggcaacgugcuuauuacugacaccuggaucuccaugggccaggaagaagagaaaaagaagcggcugcaggcguuccagggauaucaggucaccaugaaaaccgccaaggucgcugccuccgacuggaccuuccugcacugccugccucgcaagccugaagaaguggacgacgagguguucuacucgccacggagccucguguuccccgaggccgagaauagaaaguggaccaucauggccgugauggugucacugcucaccgacuacagcccgcagcuucagaagcccaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 35 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuuuucaaccugagaauccuccugaacaacgccgccuuccgcaauggucauaacuucaugguccgcaacuuucgcugcggacagccucuccaaaacaagguccagcucaaggggcgcgaccuccucacacugaagaacuucacuggagaagaaaucaaguacaugcuguggcugagcgccgaucugaaguuccggaucaagcagaagggagaguaccuuccucugcugcaagggaaguccuugggaaugauuuucgagaagcgguccacccggaccaggcugagcacugaaacuggcuucgcccugcugggaggccacccuuguuuccugaccacucaggacauccaccugggcgugaacgagucccugaccgauacugccagagugcuguccuccauggccgacgccgugcucgcccggguguacaagcagucagaccucgauacgcuggccaaggaagccuccauucccauuaucaauggucugucggaccucuaccauccaauccaaauccucgccgacuaccugacucugcaagaacacuacagcucacucaagggccucacccucuccuggaucggcgacggaaacaacauccuucacucgauuaugaugucggccgcgaaguucgggaugcaccuccaagcugccacuccaaaaggcuacgagccggaugccucagugacuaaguuggcggaacaguaugcgaaggagaacgguaccaagcuccugcugacuaacgacccgcuggaggccgcccacgggggaaacgugcucaucaccgauacuuggauuuccaugggacaggaggaagagaagaagaagcgguugcaggcauuucagggcuaccaggucaccaugaaaacugccaaagucgccgccagcgacuggaccuuccugcacugccugccccgcaagccugaagaaguggacgacgagguguucuacucuccccggucccucguguucccugaggccgaaaacaggaaguggaccaucauggcugugauggugucccuccugaccgacuacagcccucagcuccaaaaacccaaguuuuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 36 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuuuucaaccugagaauccucuugaacaaugcugcuuuucggaauggccacaacuuuaugguucggaacuuccguugcggccagccuuuacaaaacaagguccagcugaagggccgggauuugcucacacugaagaacuuuacuggggaggagauuaaguauaugcuguggcuguccgcugaccugaaguuuaggaucaagcagaagggcgaauaucugccgcugcugcaagggaaaagucugggcaugauuuuugaaaagcgcucuacccggaccagacugucuacggaaacaggcuuugcccugcugggcggccaccccuguuuucugacaacgcaggacauccaucugggcgugaacgaaucacugaccgauacugcucggguacucaguucuauggcugacgcagugcuggcuaggguguacaagcagagcgacuuggacacacuggcuaaggaggccagcauccccauuaucaauggccugucugauuuguaccaucccauucaaauccuggcugauuaucugacacuacaagagcauuacucaagucugaaggguuugacucucuccuggaucggcgacggcaacaacauuuuacauuccauuaugaugagugcugcuaaguuuggcaugcauuugcaagcugcuaccccaaagggcuaugaaccugacgcuagcguaaccaaguuggccgaacaguaugcuaaagagaauggcaccaagcugcuccugacgaaugacccccuggaagcugcucauggcggaaacguacuuauaacugauacauggauuagcaugggccaggaagaggagaagaagaagagacugcaggccuuccaaggcuaucaggucaccaugaaaacugccaagguugcagcuagcgacuggaccuuccugcacuguuugccgaggaaacccgaggagguggacgaugaagucuuuuauucuccccgcuccuugguguuucccgaggcugaaaaucgaaaguggacgauaauggcagugauggugucccuacugaccgacuauucuccacaacugcagaagccuaaauucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 37 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuuuucaaucugaggauccugcugaacaacgcugcuuuucgcaacggucauaacuuuaugguucgcaauuuucguuguggccagccgcugcagaacaagguucagcugaagggcagagaucugcugacucugaagaacuucacuggggaagaaaucaaguauauguuauggcuguccgcggaucugaaauuucgaaucaagcagaagggcgaauaucuuccccugcugcaagggaaauccuugggcaugauuuuugagaagaggagcacuaggacuagauugucaacagaaacaggcuuugcuuuguugggcggacaucccugcuuucugacgacacaggauauccaccucggcguaaacgagucccucaccgacacugcuaggguacugagcagcauggccgacgcugugcuagcccggguuuacaagcagucagaccuggacacccuugccaaggaagcuucuauuccaauuaucaacggccugagugaccuguaucacccuauucaaauacucgccgacuauuugacgcuucaagaacauuacagcagccucaagggcuuaaccuugaguuggauaggcgacggcaacaauauccugcauuccauuaugaugucugccgcuaaguuuggcaugcaucuacaagccgcaacacccaagggcuaugaacccgacgcuagcgugaccaagcuggccgagcaguaugcuaaggaaaauggcacaaagcuccuucuuaccaacgauccccuggaggcugcucacggcggcaacgugcugauuaccgauacauggauuagcaugggccaggaggaggagaaaaagaagcggcuccaggcuuuucaaggcuaucaggucaccaugaaaacugcaaaggucgcugccuccgacuggacuuuccugcauugucuaccccgcaagccugaggaaguggacgaugagguguucuacuccccacggagucugguguucccggaagcagagaaucggaaguggaccaucauggcugucauggugucgcucuugacugacuauucuccccaacugcaaaaacccaaguuuuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 38 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccauguuauucaaccuucguauccugcuaaacaaugcugcuuuucgcaauggccauaacuuuaugguucgcaacuuuagaugcggccagccgcugcagaacaagguucagcugaagggccgggacuugcugacgcugaaaaacuuuaccggggaagagauuaaguauaugcuguggcuaagcgcugaucugaaguuuaggaucaagcagaagggcgaauaucugccacugcugcaagggaagagucuuggcaugauuuuugaaaagcggucuaccagaacccggcugucgaccgagacagguuuugcucugcuggggggccaucccuguuuucugacaacucaggacauucaccugggcgugaaugagucccugaccgauacugcuaggguguugaguagcauggccgacgcuguacucgcucgaguguauaagcagucugaucuggacacucuggcuaaggaagcuuccauuccuauuaucaacggcuugagcgaccuguaccaccccauucaaauccucgcugauuacuugacuuugcaagaacauuacagcagcuugaagggcuuaacacugagcuggauaggcgacggaaacaacaucuugcauuccauaaugauguccgccgcuaaguucgggaugcaccuccaagcagccacacccaagggcuaugaaccggaugcuuccgugacaaaacuggcugagcaguaugcuaaggagaauggcacgaaacugcugcucaccaacgacccauuggaagcugcacaugguggcaacguacugaucacugacacuuggaucucaaugggccaggaggaagagaagaagaaaaggcugcaggcauuucagggauaccaagucacuaugaaaacugccaaggucgcugccuccgacuggacauuccugcauugucugccacggaagccugaggaagucgaugacgaaguguucuauagcccacgaagcuugguguuucccgaggcugagaauaggaaguggaccauuauggcuguuauggugucccugcucaccgacuauuccccucaacugcaaaaacccaaguuuuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 39 - RNA, 1365 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaugcuuuuuaaucuccgcauccuccuuaacaacgccgcguuuagaaacggccacaacuucaugguccggaacuucagauguggccagccgcuucaaaacaagguccagcugaagggccgggaucuucugacccugaagaacuuuacuggcgaagagaucaaguacaugcucuggcucuccgcggacuugaaguuccgcauuaagcagaagggggaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucuggacacucuggccaaggaggcgucaauucccaucaucaacggccugagcgaccuguaccacccaauccaaauccuggcugacuaccugacccugcaagagcacuacagcagccugaagggucugacccugucauggauuggcgauggaaacaauauucugcacuccaucaugauguccgccgcgaaguucggaaugcaucugcaagccgccacgccaaaaggauacgaaccggaugcguccgugacgaaguuggcggaacaguacgcgaaggagaacggaaccaagcuucugcugacuaacgacccccucgaggcugcgcaugggggcaacgugcugauuaccgacaccuggaucuccauggggcaggaggaagagaagaagaagagacugcaggcauuccagggguaccaggucaccaugaaaaccgcaaaaguggcagcuucggacuggacuuuccugcauugccugccgaggaagccggaggaagucgacgacgaaguguucuacucgccucggucccugguguuccccgaggccgaaaaccggaaguggaccaucauggccgugaugguguccuugcugacugacuauagcccgcagcugcagaagccuaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 40 - RNA, 1365 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaugcuuuucaaccugagaauccucuugaacaaugcugcuuuucggaauggccacaacuuuaugguucggaacuuccguugcggccagccuuuacaaaacaagguccagcugaagggccgggauuugcucacacugaagaacuuuacuggagaagagaucaaguacaugcuguggcugucggccgaccugaaguucaggaucaagcagaagggagaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucucgauaccuuggcaaaggaggcuuccauucccaucaucaacggccugagcgaccuguaccacccaauccaaauccuggcugacuaccugacccugcaagagcacuacagcagccugaagggucugacccugucauggauuggcgauggaaacaauauucugcacuccaucaugauguccgccgcgaaguucggaaugcaucugcaagccgccacuccaaaaggauacgaaccggaugcguccgugaccaaguuggcggaacaguacgcgaaggagaacggaaccaagcuucugcugacuaacgacccccucgaggcugcgcaugggggcaacgugcugauuaccgacaccuggaucuccauggggcaggaggaagagaagaagaagagacugcaggcauuccagggguaccaggucaccaugaaaaccgcaaaaguggcagcuucggacuggacuuuccugcauugccugccgaggaagccggaggaagucgacgacgaaguguucuacucgccucggucccugguguuccccgaggccgaaaaccggaaguggaccaucauggccgugaugguguccuugcugacugacuauagcccgcagcugcagaagccuaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 41 - RNA, 1365 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaugcuuuucaaccugagaauccucuugaacaaugcugcuuuucggaauggccacaacuuuaugguucggaacuuccguugcggccagccuuuacaaaacaagguccagcugaagggccgggauuugcucacacuaaagaacuuuacuggagaagagaucaaguacaugcuauggcugucggccgaccugaaguuccguaucaagcagaagggagaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucucgauaccuuggcaaaggaggcuuccauucccaucaucaacggccugagcgaccuguaccacccaauccaaauccuggcugacuaccugacccugcaagagcacuacagcagccugaagggucugacccugucauggauuggcgauggaaacaauauucugcacuccaucaugauguccgccgcgaaguucggaaugcaucugcaagccgccacuccaaaaggauacgaaccggaugcauccgugaccaaguuggcggaacaguacgcgaaggagaacggaaccaagcuccugcugacuaacgacccgcucgaggcugcgcauggggguaacgugcugauuacggacaccuggaucuccauggggcaggaggaagagaagaagaagagacugcaggcauuccagggguaccaggucaccaugaaaaccgcaaaaguggcagcuucggacuggacuuuccugcauugccugccgaggaagccggaggaagucgacgacgaaguguucuacucgccucggucccugguguuccccgaggccgaaaaccggaaguggaccaucauggccgugaugguguccuugcugacugacuauagcccgcagcugcagaagccuaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 42 - RNA, 1365 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaugcuuuucaaccugagaauccucuugaacaaugcugcuuuucggaauggccacaacuuuaugguucggaacuuccguugcggccagccuuuacaaaacaagguccagcugaagggccgggauuugcucacacuaaagaacuuuacuggagaagagaucaaguacaugcuauggcugucggccgaccugaaguuccguaucaagcagaagggagaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucucgauaccuuggcaaaggaggcuuccauucccaucaucaacggccugagcgaccuguaccacccaauccaaauccuggcugacuaccugacccugcaagagcacuacagcagccugaagggucugacccugucauggauuggcgauggaaacaauauucugcacuccaucaugauguccgccgcgaaguucggaaugcaucugcaagccgccacuccaaaaggauacgaaccggaugcguccgugaccaaguuggcggaacaguacgcgaaggagaacggaaccaagcuucugcugacuaacgacccccucgaggcugcgcaugggggcaacgugcugauuaccgacaccuggaucuccauggggcaggaggaagagaagaagaagagacugcaggcauuccagggguaccaggucaccaugaaaaccgcaaaaguggcagcuucggacuggacuuuccugcauugccugccgaggaagccggaggaagucgacgacgaaguguucuacucgccucggucccugguguuccccgaggccgaaaaccggaaguggaccaucauggccgugaugguguccuugcugacugacuauagcccgcagcugcagaagccuaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 43 - RNA, 1371 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuguucaaccugcgaauccugcugaacaacgccgcuuuucggaacgggcacaacuuuauggugaggaacuuucgcugcggacagccccuccagaauaagguccagcugaagggcagggaccugcugacccugaaaaauuucacaggggaggaaaucaaguauaugcuguggcugucagcugaucugaaguuccggaucaagcagaagggcgaauaucugccucugcuccagggcaaaagccuggggaugaucuucgaaaagcgcaguacucggaccagacugucaaccgagacuggauucgcucugcugggaggacacccuuguuuucugaccacucaggacauucaccugggagugaacgagucccugaccgacacugcucgcguccugagcucuauggccgacgcugugcuggcucgagucuacaaacaguccgaccuggauacccuggccaaggaagcuucuaucccaauuauuaacggccugucagaccuguaucaccccauccagauucuggccgauuaccugacccuccaggagcacuauucuagucugaaagggcugacacugaguuggauuggggacggaaacaauauccugcacucuauuaugaugucagccgccaaguuuggaaugcaccuccaggcugcaaccccaaaaggcuacgaacccgaugccucagugacaaagcuggcugaacaguacgccaaagagaacggcacuaagcugcugcugaccaacgacccucuggaggccgcucacggaggcaacgugcugaucaccgauaccuggauuaguaugggacaggaggaagagaagaagaagcggcuccaggccuuccagggcuaccaggugacaaugaaaaccgcuaaggucgcagccagcgauuggaccuuucugcacugccugcccagaaagcccgaagagguggacgacgaggucuucuacucucccagaagccugguguuucccgaagcugagaauaggaaguggacaauuauggcagugauggucagccugcugacugauuauucaccucagcuccagaaaccaaaguucugauaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 44 - RNA, 1371 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuguucaaccugcgcauccugcugaacaacgccgccuuccgcaacggccacaacuucauggugcgcaacuuccgcugcggccagccccugcagaacaaggugcagcugaagggccgcgaccugcugacccugaagaacuucaccggcgaggagaucaaguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggccuggcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugauaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 45 - RNA, 1371 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuguucaaccugcgaauccugcugaacaacgccgcuuuucggaacgggcacaacuuuauggugaggaacuuucgcugcggacagccccuccagaauaagguccagcugaagggcagggaccugcugacccugaaaaauuucacaggggaggaaaucaaguauaugcuguggcugucagcugaucugaaguuccggaucaagcagaagggcgaauaucugccucugcuccagggcaaaagccuggggaugaucuucgaaaagcgcaguacucggaccagacugucaaccgagacuggauucgcucugcugggaggacacccuuguuuucugaccacucaggacauucaccugggagugaacgagucccugaccgacacugcucgcguccugagcucuauggccgacgcugugcuagcucgagucuacaaacaguccgaccuggauacccuggccaaggaagcuucuaucccaauuauuaacggccugucagaccuguaucaccccauccagauucuggccgauuaccugacccuccaggagcacuauucuagucugaaagggcugacacugaguuggauuggggacggaaacaauauccugcacucuauuaugaugucagccgccaaguuuggaaugcaccuccaggcugcaaccccaaaaggcuacgaacccgaugccucagugacaaagcuggcugaacaguacgccaaagagaacggcacuaagcugcugcugaccaacgacccucuggaggccgcucacggaggcaacgugcugaucaccgauaccuggauuaguaugggacaggaggaagagaagaagaagcggcuccaggccuuccagggcuaccaggugacaaugaaaaccgcuaaggucgcagccagcgauuggaccuuucugcacugccugcccagaaagcccgaagagguggacgacgaggucuucuacucucccagaagccugguguuucccgaagcugagaauaggaaguggacaauuauggcagugauggucagccugcugacugauuauucaccucagcuccagaaaccaaaguucugauaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 46 - RNA, 1371 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuguucaaccugcgaauccugcugaacaaugccgcuuuucggaacgggcacaauuucauggugaggaacuuucgcugcggacagccccuccagaacaagguccagcugaagggcagggaccugcugacccugaaaaauuucacaggggaggaaaucaaguacaugcuguggcugucagccgaucugaaguuccggaucaagcagaagggcgaauaucugccucugcuccagggcaaaagccuggggaugaucuucgaaaagcgcaguacucggaccagacugucaacagagacuggauucgcacugcugggaggacacccauguuuucugaccacacaggacauucaucugggagugaacgagucccugaccgacacagcacgcguccugagcuccauggcugaugcagugcuggcucgagucuacaaacagucugaccuggauacccuggccaaggaagcuucuaucccaaucauuaauggccugagugaccuguaucaccccauccagauucuggccgauuaccugacccuccaggagcauuauucuagucugaaagggcugacacugagcuggauuggggacggaaacaauauccugcacuccauuaugaugagcgccgccaaguuuggaaugcaccuccaggcugcaaccccaaaaggcuacgaacccgaugccuccgugacaaagcuggcagaacaguaugccaaagagaacggcacuaagcugcugcugaccaaugacccucuggaggccgcucacggaggcaacgugcugaucacugauaccuggauuaguaugggacaggaggaagagaagaagaagcggcuccaggccuuccagggcuaccaggugacaaugaaaacugcuaaggucgcagccagcgacuggaccuuucugcauugccugcccagaaagccugaagagguggacgaugaggucuucuacucacccagaagccugguguuuccugaagcugagaauaggaaguggacaaucauggcagugauggucagccugcugacugauuauuccccucagcuccagaaaccaaaguucugauaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 47 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuuuucaaccuucgcauucuccucaacaacgccgcguuuagaaacggacacaacuucaugguccgcaacuuccgcugcggacagccgcugcagaacaagguccagcucaagggucgggaucuccugacgcugaagaacuuuaccggcgaagagauuaaguacaugcuguggcuguccgccgaccuuaaguuccggaucaagcagaagggcgaauaccuuccccugcugcaaggaaagucccugggcaugaucuucgagaagcgcaguaccagaaccagacucuccacugaaaccggguucgcgcugcuuggcggccacccguguuuccucacuacgcaagacauccaucuuggcgugaacgagucccuuaccgacaccgccagggugcugucaagcauggccgacgccguccuugcgcgcguguacaagcagucagaccuugauacucuggccaaggaagccuccaucccuauuaucaacggccuauccgaccuuuaccacccgauccagauccucgcugacuaccugacccugcaagaacacuacagcagccucaagggacugacucuguccuggaucggcgacgggaacaacauccugcacucaaucaugaugagcgcagccaaguucggcaugcaucuccaagccgcuacacccaaggguuaugaaccggacgccucugugaccaaguuggcagaacaguacgccaaggagaacgguacuaagcuccuuuuaaccaacgacccccucgaagcagcccauggcgggaaugugcucauuaccgauaccuggauuucgaugggccaggaggaggagaagaagaagcggcugcaggcguuccagggcuaccaggucaccaugaaaacugccaaaguggccgccucggauuggaccuuucuccacugccugccucggaagccugaggagguggacgacgaaguguucuacuccccacggucccucguguuccccgaggccgaaaauaggaaguggaccaucauggccgugauggugucccucuugaccgauuacagcccgcagcuucagaagccuaaauucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 48 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuuuucaaucuucgcauccuguugaacaacgccgccuuccgcaauggucacaacuucaugguccggaacuucagauguggacagccucuccaaaacaagguccagcugaagggaagggaccucuuaacccucaaaaacuuuacuggagaggagaucaaguacaugcuguggcuuagcgccgaccuuaaguuccggaucaagcagaagggagaguaccucccgcugcugcaaggaaagagucuuggaaugaucuucgagaagcgguccaccagaacucgccucuccacugaaaccggauucgcacuccuggguggacacccgugcuuucugaccacccaagacauccaccucggagugaacgagagccucacggacaccgcgagagugcugucauccauggccgacgccgugcuugcacgggucuacaagcaguccgaucuggacacucuugccaaggaagccuccauuccuaucauuaacggucugucggaucuguaccacccgauucagauccuugcggacuaccucacacuucaagaacacuauucaagccuaaagggucugacccuguccuggaucggagauggaaacaacauucuccauuccaucaugaugagcgcugccaaguucggaaugcaucuccaagcagcgacuccuaaggguuacgagccggacgccucagugacuaagcuggccgagcaguacgccaaggagaacgguaccaaacuguugcuuacuaacgacccgcuugaagcggcccauggaggaaacgugcugauuaccgacaccuggauuucgaugggacaggaagaggagaagaagaagcggcuccaggcguuccagggauaccaggucaccaugaaaacggccaaaguggccgcuagcgauuggaccuuucugcacugccucccgcgcaagccugaagaaguggacgacgaaguguucuacuccccucgcucucuuguguucccggaagccgaaaacaggaaguggaccaucauggccgugauggugucccuccugaccgauuacagcccgcagcugcagaagccuaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 49 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuuuucaaucuccgcauccuccucaacaacgccgcguuuagaaacggccacaacuucaugguccggaacuucagauguggccagccgcuucagaacaagguccagcucaagggccgggaucuucugacccugaagaacuuuacuggcgaagaaaucaaguacaugcucuggcucuccgccgacuugaaguuccgcauuaagcagaagggggaauaccuuccgcugcugcaaggaaagucgcucggcaugaucuuugagaagcgcucaacccgcaccaggcuguccacugaaaccggguucgcgcugcuugguggccaccccugcuuccugaccacccaagacauucaccucggagugaacgaaucgcucacugauacugcccgggugcugucgucgauggccgaugcagugcuggccaggguguacaaacaguccgaucuggacacucuggccaaggaggcguccaucccuauuaucaacggccuuuccgaccucuaccacccgauucagauccuugccgauuaccucacccugcaagaacacuacucgucacugaagggucugaccuuguccuggaucggcgacggcaacaacauccuccauuccauuaugauguccgccgccaaauucggcaugcaucuucaagccgcaaccccuaaggguuacgagccggacgcuuccgugaccaagcucgccgagcaguacgcuaaggagaacggaaccaagcuucugcugacuaacgacccccuagaggcagcccacgggggcaacgugcuuauuacugacaccuggaucuccaugggacaggaagaagagaagaagaagcgguuacaggcguuccagggcuaucaggucaccaugaaaaccgccaaggucgcugccucggacuggaccuuccugcauugccugccucgcaagcccgaagaaguggacgacgagguguucuacucgccacggucccuuguguucccugaggccgagaauagaaaguggaccauuauggccgugauggugucccuucucaccgacuacucgccgcaacugcagaaacccaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 50 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuuuucaaucuucgcauccuccucaacaacgccgccuuccggaacggucacaacuucaugguccggaacuuccgcugcggccagccgcuccaaaacaaagugcagcuuaagggccgcgaucuccugacccugaagaacuucaccggagaggaaaucaaguacaugcuguggcucucggcggaccugaaguuuaggauuaagcagaagggggaguaucugccgcugcuccaagggaagucccuuggcaugaucuucgaaaagagguccacccggacucggcucagcaccgaaacagguuuugcacuucuggggggccacccgugcuuccugacgacccaggacauccaucugggugucaacgagaguuugaccgacacugccagagugcugucauccauggcggacgcggugcucgcgagaguguacaagcaguccgaucuugacacccuggcaaaagaggcuucaaucccgaucauuaacggacucucggaucuguaccacccuauccaaaucuuggccgacuaccugacccugcaagaacacuacagcucccugaagggccugacucuuuccuggauuggcgauggaaacaacauucuccauucuauuaugauguccgccgccaaguucggcaugcaccuucaagccgccaccccgaagggcuacgaaccugacgccuccgugacuaagcuagccgaacaguacgcuaaggagaacggcacuaagcuucuccuuaccaacgauccgcuggaggcggcccauggcggaaaugugcuuaucaccgacaccuggauuagcauggggcaggaagaagagaagaagaaacggcuccaggcauuccagggcuaccaggucaccaugaaaacugccaaggucgccgcuagcgacuggaccuuccuccacugucugccucgcaagccugaagaaguggacgacgagguguucuacuccccgcgcucccucguguuuccugaggccgagaacagaaaguggaccaucauggccgugauggugucauuacuuacggacuacagcccgcagcugcagaagccgaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 51 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuuuuuaacuugagaauccuucugaacaacgccgcuuuccgcaacggucauaacuucaugguccggaacuucagauguggccagccccuccaaaacaaagugcagcugaagggccgggaccuucuuacgcugaagaauuucaccggcgaagaaaucaaguacaugcucuggcuguccgccgaucuuaaguuccgcauuaagcagaagggggaauaccucccgcugcugcaagggaagucgcugggcaugauuuuugagaagcggucaacucgcacccgccuguccacugaaacuggauucgcacugcucgguggccaucccugcuuccugaccacccaagacauccaccucggcgugaacgagucccugacugacaccgcccgggucuuauccucgauggccgaugcugugcuugcgaggguguacaagcaguccgaccucgacacacucgcgaaggaggccuccauccccaucaucaacggccuguccgaccuuuaccacccaauucagauccucgccgauuaccugacccugcaagagcacuacucgucgcucaaggggcuuacccucucguggauuggcgacggcaacaacauccuucacuccaucaugaugucggcagcgaaguucggcaugcaucugcaagccgccacgccuaaggguuaugaaccggaugccucagugaccaagcucgccgaacaguacgcgaaagagaauggaaccaagcuacuucugaccaacgacccccuggaggccgcucacggcggcaacguccucauuaccgauacuuggauuucgaugggacaggaagaggaaaagaagaagagacugcaggcguuccagggauaccaggucaccaugaaaacugccaaaguggcagccuccgacuggaccuuccuucacugccugccgaggaagccugaagagguggacgacgagguguucuacuccccgcgcuccuugguguuuccugaggccgaaaaccggaaguggacuaucauggccgugauggugucccuccucaccgacuacucgccgcaacugcagaagccuaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 52 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccauguuauucaaccuuagaauucuccuuaacaacgccgccuuccggaaugggcauaacuuuaugguccgcaauuuccgcuguggacagccucugcaaaacaagguccagcucaagggccgggaucugcugacucucaagaacuucacuggggaagaaaucaaguacaugcucuggcugagcgccgaccucaaguuccgcaucaagcagaagggagaguaccucccgcugcuccaagggaagucccugggcaugaucuucgagaagagauccacccgcaccagacuuuccacugagacuggcuucgccuugcugggaggccacccaugcuuccugacgacccaggacauucaccuuggcgugaacgagucccugacugacaccgcaaggguguuguccucgauggccgacgccgugcuugcccggguguacaagcagagcgaucuugacacccuggcuaaggaagcuuccauucccaucaucaacggucugagcgaccuguaccacccgauucagauccuggcggacuaccuaacccugcaagagcacuauagcucccugaagggccucacacuuucauggaucggcgacggcaacaacauccugcacucuauuaugaugagcgcugccaaauucggcaugcaccuccaagccgccacgccuaaaggcuacgagcccgacgccucggugaccaagcuugcggagcaguacgcgaaggaaaacggcaccaagcugcuucucaccaacgauccucuggaagcggcccaugguggcaacgugcucauuaccgacacuuggaucuccaugggacaggaggaggaaaagaagaagcggcuccaggcguuucaggguuaccaggucaccaugaaaaccgccaaggucgcagccuccgacuggaccuuccuucauugccuuccgcgcaagcccgaagaaguggacgaugaaguguuuuacucaccucggucacucguguucccggaagcagagaacaggaaauggaccauuauggccgugauggugucccugcucaccgauuacaguccgcaacugcagaagcccaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 53 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuuuucaaucuccgcauccuccuuaacaacgccgcguuuagaaacggccacaacuucaugguccggaacuucagauguggccagccgcuucaaaacaagguccagcugaagggccgggaucuucugacccugaagaacuuuacuggcgaagagaucaaguacaugcucuggcucuccgcggacuugaaguuccgcauuaagcagaagggggaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucuggacacucuggccaaggaggcgucaauuccuauuaucaacggccuuagugaccucuaccauccgauucaaauccuggccgauuaccucacccugcaagaacacuacagcucccugaagggucugacauuguccuggaucggcgacggcaacaacauucuccauuccaucaugauguccgccgcaaaauucggcaugcaucuucaagccgccacgccuaaggguuacgaacccgacgcuuccgugacuaagcucgccgagcaguacgcuaaggagaacggaaccaagcugcugcugacuaacgacccgcuagaagcagcccacgggggcaacgugcuuauuacugacaccuggaucuccaugggccaggaggaagagaaaaagaagcggcugcaggcguuccagggauaucaggucaccaugaaaaccgccaaggucgcugccuccgacuggaccuuccugcacugccugccucgcaagccugaagaaguggacgacgagguguucuacucgccacggagccucguguuccccgaggccgagaauagaaaguggaccaucauggccgugauggugucacuucucaccgacuacagcccgcagcuucagaagcccaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 54 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuuuucaaucuccgcauccuccuuaacaacgccgcguuuagaaacggccacaacuucaugguccggaacuucagauguggccagccgcuucaaaacaagguccagcuuaagggccgggaucuccucacccuuaaaaacuucaccggcgaagagaucaaguacaugcucuggcucuccgcggaccuuaaguuccgcauuaagcagaagggggaauaccuuccgcugcuucaaggaaagagccucggcaugaucuuugagaagcgcucaaccaggaccaggcuuucuacugaaacuggguucgcgcuucucggcggucaucccugcuuccucacgacccaagacauccaccucggagugaacgaaucccucacggauacugcccgcgugcuuucgagcauggcagacgccgugcucgcccggguguacaaacaguccgaucucgacacucucgccaaggaggcgucaauuccuauuaucaacggucuuagugaccuuuaccacccgauccagauccucgccgauuaccucacacuccaagaacacuacagcucccuuaagggucuuacccucuccuggaucggcgacggcaacaacauucuccacuccaucaugauguccgccgcaaaguucggcaugcaucuucaagccgccaccccgaagggcuacgagccugaugcuuccgugacuaagcucgccgagcaguacgcuaaggagaacggaaccaagcuucuucucacuaacgacccacucgaagcagcccaugggggcaacgugcuuaucacugacaccuggaucuccaugggccaggaagaagagaagaagaagcggcuccaggcguuccagggauaucaggucaccaugaaaaccgccaaggucgcugccuccgacuggaccuuucuccacugccucccucgcaaaccugaagaaguggacgacgagguguucuacucgccccggagccucguguuccccgaggccgagaauagaaaguggaccauuauggccgugauggugucacuccucaccgacuacagcccgcagcuucagaagcccaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 55 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuuuucaaucuccgcauccuccuuaacaacgccgcguuuagaaacggacauaacuucaugguccggaacuucagauguggacagccgcuucaaaacaagguccagcugaagggucgggaucuucugacccugaagaacuuuaccggagaagagaucaaguacaugcucuggcucuccgcggacuugaaguuccgcauuaagcagaagggagaauaccucccgcugcuucaaggaaagagccucggaaugauuuuugagaagcgcucaaccaggacccgccuuucuacugaaacuggauucgcgcugcuggguggacaccccugcuuccugacgacccaggacauccaccucggagugaacgaaucccucacugauaccgcccggguguuaucgagcauggcagaugccgugcuggccaggguguacaaacaguccgaucuggacacucuggccaaggaggcgucaauuccuaucaucaacggacuuagugaccucuaccauccgauucaaauccuggccgacuaccucacccugcaagaacacuacagcucccugaagggucugacauuguccuggaucggagauggaaacaacauucuccacuccaucaugauguccgccgcaaaauucggaaugcaucuucaagccgccacgccuaaggguuacgaacccgacgcuuccgugacuaagcucgccgagcaguacgcuaaggagaacgguaccaagcuucuccugaccaacgacccacuagaagcagcccacgguggaaacgugcuuauuacugacacuuggaucuccaugggacaggaggaagagaaaaagaagcggcugcaggcguuccagggauaucaggucaccaugaaaaccgccaaggucgcugccuccgacuggaccuuccugcacugccugccucgcaagccugaagaaguggacgacgagguguucuacucgccgcggagccucguguuccccgaggccgagaauagaaaguggaccaucauggccgugauggugucacugcucaccgacuacagcccgcagcuucagaagcccaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 56 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuuuucaaccuccgcauucuccucaacaacgcugccuuccggaauggacauaacuucaugguccggaacuucagaugcggacagccgcuucagaacaagguccagcuuaaggggagagaucuccuuacccucaaaaacuucacuggcgaagaaaucaaguacaugcucuggcuuagugcggaucucaaguuccgcaucaagcagaagggagaauaccucccgcuccuucaaggaaagagccucggcaugauuuuugagaagagguccaccagaacucgccuuucaaccgagacuggguucgcccugcuuggcggucaccccugcuuccucacuacccaagacauccaccucggcgugaacgagagccuuaccgacaccgcccgcgugcucuccucaauggccgacgcugugcucgcccggguguacaagcaguccgaccuugauacucucgccaaggaggccuccaucccaauuaucaacgggcucucugaucucuaccacccuauccaaauccucgcggacuaccucacccuccaagagcacuauagcucgcucaagggccucacccuuuccuggauuggcgacggcaacaacauucuucacucgaucaugauguccgccgccaaguucggcaugcaucuccaagccgcgacccccaagggcuacgagccugacgcauccgugaccaagcucgccgagcaguacgcgaaggaaaauggcaccaagcuucuucucaccaacgacccccuugaggccgcucauggcggcaacgugcucaucacugacacuuggaucagcaugggccaggaggaggaaaagaagaagcgccuucaggcauuccaggguuaccaggucaccaugaaaaccgccaaaguggccgccuccgacuggaccuuucuucacugucucccgcggaagccugaagaaguggaugacgaaguguuuuacuccccucggucacucguguucccggaagcagaaaacaggaaguggaccauuauggcggucauggugucccuccucaccgacuacagcccgcagcuucagaaacccaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 57 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuuuucaaucuccgcauccuccuuaacaacgcagcguuuagaaacggucacaacuucaugguccggaacuuccgcuguggacagccgcuucaaaacaagguccagcugaagggucgggaccuucugacccugaagaacuuuacuggagaagagaucaaguacaugcuuuggcuguccgcggacuugaaguuccgcauuaagcagaagggagaauaccuuccgcugcuccaaggaaagagccugggaaugaucuuugagaagcgcucaaccaggacccgccuuucuacugaaacuggauucgcgcugcuggguggucacccuugcuuccugacgacccaggacauucaccucggagugaacgagucccucacugauaccgccagaguguuaucgagcauggcagaugccgugcuggcuaggguguacaaacaguccgaucuggacacccuggccaaggaggcaucaauuccuauuaucaacggacuuagugaccucuaccauccgauucaaauccuggccgauuaccucacccugcaagaacacuacagcucccugaagggucugacauuguccuggaucggagauggaaacaacauucuccauuccaucaugauguccgcggccaaguucggaaugcaucuccaagccgccacgccgaaaggauacgagccggacgcuuccgugacuaagcucgccgagcaguacgcuaaggagaacggaaccaagcuucugcugacuaacgacccgcuagaagccgcccacgguggaaacgugcuuauuacugacaccuggaucuccaugggacaggaagaagagaaaaagaagcggcugcaggcguuccagggauaucaggucaccaugaaaaccgccaaggucgccgccuccgacuggaccuuccuucacugccugccucggaagccugaagaaguggacgacgagguguucuacucgccgcggagccucguguucccugaggccgagaauagaaaguggaccaucauggccgugauggugucacuccucaccgacuacagcccgcagcuucagaagccuaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 58 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuuuucaaucuccgcauucuccucaacaacgcagccuuuagaaacggccacaacuucaugguccggaacuucagauguggccagccgcuucagaacaagguccagcucaagggccgggaccuccucacccucaaaaacuuuaccggcgaagagaucaaguacaugcucuggcuuucggccgaccuuaaguuccgcaucaagcagaagggggaauaccuuccgcugcuucaaggaaagucccucggcaugaucuuugaaaagcgcucgaccaggacccgccuuuccacugaaaccggguucgcgcuucucgguggccaccccugcuuccucaccacccaagacauucaccucggagugaacgaaucccuuaccgauaccgcaagagugcuuucgucgauggccgaugccgugcuugcgcggguguacaagcagucagaucucgacacucucgccaaggaggcguccauuccuauuaucaacggccuuuccgaccuuuaccacccgauucagauccucgccgauuaccucacccugcaagagcacuacucgucacucaagggucuuacccucuccuggaucggcgacggaaacaacauccuccauucgaucaugauguccgccgccaaauucggcaugcaccuccaagccgcgaccccgaaggguuacgagcccgacgcuuccgugaccaagcucgccgaacaguacgcuaaggaaaacggcaccaagcuccuccucacuaacgacccucucgaagcagcccaugggggcaacgugcucauuacugacacuuggaucucgaugggccaggaagaggagaaaaagaagcggcuucaggcguuccagggauaucaggucaccaugaaaaccgccaaggucgcugccucggacuggaccuuccuucacugccuuccgcgcaagccugaagagguggacgaugagguguucuacuccccacggucccuuguguuccccgaggccgagaauaggaaguggaccaucauggccgugauggugucgcuccucacugacuacuccccgcaacuucagaagccuaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 59 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuguuuaaucugagaauacuucuaaacaacgccgccuuccggaauggccauaacuuuaugguucggaauuuccgcugcggccagccgcugcagaacaagguccagcugaagggaagagacuugcugacccucaagaacuucaccggagaagaaaucaaguauaugcuguggcuguccgccgaccugaaauuccgcaucaagcagaagggcgaauaucugccgcuguugcaagggaagucccuggggaugaucuucgagaagagguccaccagaacacggcuuucaaccgaaaccggguuugcacugcuggguggacaccccuguuuucugaccacucaagauauccaccugggcgugaacgagucccuuaccgacacugcuaggguguuguccagcauggccgaugccguccuggcucgcguguacaagcaguccgaccuggauacccuggcaaaggaagcguccauucccauuaucaacgggcuguccgaccuguaccauccgauucaaauccuggcggacuaccugacucugcaagagcauuacagcagcuugaaggggcuuacucucucguggaucggcgacgggaacaacauccugcacuccaucaugauguccgccgccaaguucgggaugcauuugcaagcugcgaccccgaaagguuacgagcccgaugcuagcguaacuaagcuugccgaacaguacgccaaagagaaugguacaaaacugcuucugacuaacgacccgcuggaagcagcccacggcgggaacgugcugauaaccgacaccuggauuucaauggggcaggaggaagagaagaagaagcgacugcaggcguuccaaggcuaucagguuaccaugaaaaccgccaaaguggcagccagcgauuggacuuuccugcacugucugccgcggaagcccgaggaaguugaugacgaaguauucuacucaccccggagccucguguuccccgaggccgaaaaccggaaguggacuauuauggccgugauggugucgcuguugaccgacuacagcccgcaacugcagaagccgaaguuuuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 60 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuuuucaaccugaggauccuuuugaacaacgccgccuuucgcaacggccacaacuuuaugguccgcaauuuccgcugcgggcagccgcugcagaacaagguccagcugaagggccgggaucugcugacccugaagaacuucaccggggaggaaaucaaguacaugcuuuggcucuccgccgaucugaaguucagaaucaagcagaagggagaguaccucccguugcugcaaggaaagucacucggaaugauuuucgaaaagagaagcacuaggacccgccucucaacugaaaccggguucgcgcugcucgggggccauccguguuuccugacuacccaagacauccaccugggagugaacgagucgcugaccgacaccgcacgcgugcugucauccauggcggacgcagugcuugcccggguguacaagcagucggaccuggacacucuugccaaggaggcaucaauccccaucauuaacggacuguccgaucucuaccacccgauucagauccuggcugacuaccuaacccugcaagagcacuacucaagccugaaggggcugacccugucguggaucggggacggcaacaacauucugcacuccaucaugaugucggcggcuaaguucgggaugcauuugcaagcggcaacuccgaaggguuaugaacccgacgccuccgugaccaagcuggccgaacaguacgccaaggaaaacggaaccaaguugcugcugacuaaugauccccuggaggcggcccacggggggaacgugcugauaaccgauaccuggaucuccauggggcaggaagaagagaagaaaaagcggcugcaggcauuccagggauaccaggucaccaugaaaaccgcaaaaguggcagccagcgacuggacuuuccuccauugccugccgcgaaagccggaggaggucgaugacgagguguucuacuccccgcggucgcugguguucccggaggcggaaaaccggaaguggaccauuauggccgugauggugucacuccugacugacuacagcccgcaacugcagaagccgaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 61 - RNA, 1496 nt, Artificial Sequence
  Synthetic construct
cuuaagggggcgcugccuacggagguggcagccaucuccuucucggcaucaagcuuaccauggugccccaggcccugcucuuggucccgcugcugguguucccccucugcuucggcaaguuccccaucuacaccauccccgacaagcuggggccguggagccccaucgacauccaccaccuguccugccccaacaaccucguggucgaggacgagggcugcaccaaccugagcggguucuccuacaugcuuuucaaucuccgcauccuccuuaacaacgccgcguuuagaaacggccacaacuucaugguccggaacuucagauguggccagccgcuucaaaacaagguccagcugaagggccgggaucuucugacccugaagaacuuuacuggcgaagagaucaaguacaugcucuggcucuccgcggacuugaaguuccgcauuaagcagaagggggaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucuggacacucuggccaaggaggcgucaauuccuauuaucaacggccuuagugaccucuaccauccgauucagauccuggccgauuaccucacccugcaagaacacuacagcucccugaagggucugacauuguccuggaucggcgacggcaacaacauucuccauuccaucaugauguccgccgcaaaauucggcaugcaucuucaagccgccacgccgaaggguuacgagcccgacgcuuccgugacuaagcucgccgagcaguacgcuaaggagaacggaaccaagcuucugcugacuaacgacccacuagaagcagcccacgggggcaacgugcuuauuacugacaccuggaucuccaugggccaggaagaagagaaaaagaagcggcugcaggcguuccagggauaucaggucaccaugaaaaccgccaaggucgcugccuccgacuggaccuuccugcacugccugccucgcaagccugaagaaguggacgacgagguguucuacucgccacggagccucguguuccccgaggccgagaauagaaaguggaccaucauggccgugauggugucacugcucaccgacuacagcccgcagcuucagaagcccaaguucuagauaagugaaugcaaggcuggccggaagcccuugccugaaagcaagauuucagccuggaagagggcaaaguggacgggaguggacaggaguggaugcgauaagaugugguuugaagcugaugggugccagcccugcauugcugagucaaucaauaaagagcuuucuuuugacccauucuagaucuag

SEQ ID NO: 62 - RNA, 1368 nt, Artificial Sequence
  Synthetic construct
aggaaacuuaagauuauuacaucaaaacaaaaagccgccaaugcuguucaaccugcgcauccugcugaacaacgccgccuuccgcaacggccacaacuucauggugcgcaacuuccgcugcggccagccccugcagaacaaggugcagcugaagggccgcgaccugcugacccugaagaacuucaccggcgaggagaucaaguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggccuggcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugaacgccgaagccugcagccaugcgaccccacgccaccccgugccuccugccuccgcgcagccugcagcgggagacccuguccccgccccagccguccuccugggguggacccuaguuuaauaaagauucaccaaguuucacgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 63 - RNA, 1495 nt, Artificial Sequence
  Synthetic construct
cuuaagggggcgcugccuacggagguggcagccaucuccuucucggcaucaagcuuaccauggugccccaggcccugcucuuggucccgcugcugguguucccccucugcuucggcaaguuccccaucuacaccauccccgacaagcuggggccguggagccccaucgacauccaccaccuguccugccccaacaaccucguggucgaggacgagggcugcaccaaccugagcggguucuccuacaugcuguucaaccugcgcauccugcugaacaacgccgccuuccgcaacggccacaacuucauggugcgcaacuuccgcugcggccagccccugcagaacaaggugcagcugaagggccgcgaccugcugacccugaagaacuucaccggcgaggagaucaaguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggccuggcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugaauaagugaugcaaggcuggccggaagcccuugccugaaagcaagauuucagccuggaagagggcaaaguggacgggaguggacaggaguggaugcgauaagaugugguuugaagcugaugggugccagcccugcauugcugagucaaucaauaaagagcuuucuuuugacccauucuagaucuag

SEQ ID NO: 64 - RNA, 1368 nt, Artificial Sequence
  Synthetic construct
aggaaacuuaagauuauuacaucaaaacaaaaagccgccaaugcuuuucaaucuccgcauccuccuuaacaacgccgcguuuagaaacggccacaacuucaugguccggaacuucagauguggccagccgcuucaaaacaagguccagcugaagggccgggaucuucugacccugaagaacuuuacuggcgaagagaucaaguacaugcucuggcucuccgcggacuugaaguuccgcauuaagcagaagggggaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucuggacacucuggccaaggaggcgucaauuccuauuaucaacggccuuagugaccucuaccauccgauucagauccuggccgauuaccucacccugcaagaacacuacagcucccugaagggucugacauuguccuggaucggcgacggcaacaacauucuccauuccaucaugauguccgccgcaaaauucggcaugcaucuucaagccgccacgccgaaggguuacgagcccgacgcuuccgugacuaagcucgccgagcaguacgcuaaggagaacggaaccaagcuucugcugacuaacgacccacuagaagcagcccacgggggcaacgugcuuauuacugacaccuggaucuccaugggccaggaagaagagaaaaagaagcggcugcaggcguuccagggauaucaggucaccaugaaaaccgccaaggucgcugccuccgacuggaccuuccugcacugccugccucgcaagccugaagaaguggacgacgagguguucuacucgccacggagccucguguuccccgaggccgagaauagaaaguggaccaucauggccgugauggugucacugcucaccgacuacagcccgcagcuucagaagcccaaguucuagacgccgaagccugcagccaugcgaccccacgccaccccgugccuccugccuccgcgcagccugcagcgggagacccuguccccgccccagccguccuccugggguggacccuaguuuaauaaagauucaccaaguuucacgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 65 - RNA, 1495 nt, Artificial Sequence
  Synthetic construct
cuuaagggggcgcugccuacggagguggcagccaucuccuucucggcaucaagcuuaccauggugccccaggcccugcucuuggucccgcugcugguguucccccucugcuucggcaaguuccccaucuacaccauccccgacaagcuggggccguggagccccaucgacauccaccaccuguccugccccaacaaccucguggucgaggacgagggcugcaccaaccugagcggguucuccuacaugcuuuucaaccugagaauccucuugaacaaugcugcuuuucggaauggccacaacuuuaugguucggaacuuccguugcggccagccuuuacaaaacaagguccagcugaagggccgggauuugcucacacuaaagaacuuuacuggagaagagaucaaguacaugcuauggcugucggccgaccugaaguuccguaucaagcagaagggagaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucucgauaccuuggcaaaggaggcuuccauucccaucaucaacggccugagcgaccuguaccacccaauccaaauccuggcugacuaccugacccugcaagagcacuacagcagccugaagggucugacccugucauggauuggcgauggaaacaauauucugcacuccaucaugauguccgccgcgaaguucggaaugcaucugcaagccgccacuccaaaaggauacgaaccggaugcauccgugaccaaguuggcggaacaguacgcgaaggagaacggaaccaagcuccugcugacuaacgacccgcucgaggcugcgcauggggguaacgugcugauuacggacaccuggaucuccauggggcaggaggaagagaagaagaagagacugcaggcauuccagggguaccaggucaccaugaaaaccgcaaaaguggcagcuucggacuggacuuuccugcauugccugccgaggaagccggaggaagucgacgacgaaguguucuacucgccucggucccugguguuccccgaggccgaaaaccggaaguggaccaucauggccgugaugguguccuugcugacugacuauagcccgcagcugcagaagccuaaguucuagauaagugaugcaaggcuggccggaagcccuugccugaaagcaagauuucagccuggaagagggcaaaguggacgggaguggacaggaguggaugcgauaagaugugguuugaagcugaugggugccagcccugcauugcugagucaaucaauaaagagcuuucuuuugacccauucuagaucuag

SEQ ID NO: 66 - RNA, 1475 nt, Artificial Sequence
  Synthetic construct
ugagugucguacagccuccaggccccccccucccgggagagccauaguggucugcggaaccggugaguacaccggaauugccgggaagacuggguccuuucuuggauaaacccacucuaugcccggccauuugggcgugcccccgcaagacugcuagccgaguaguguuggguugcgaugcuguucaaccugcgcauccugcugaacaacgccgccuuccgcaacggccacaacuucauggugcgcaacuuccgcugcggccagccccugcagaacaaggugcagcugaagggccgcgaccugcugacccugaagaacuucaccggcgaggagaucaaguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggccuggcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugaauaagugauagagcggcaaacccuagcuacacuccauagcuaguuucuuuuuuuuuuguuuuuuuuuuuuuuuuuuuuuuuuuuuuuuuuuuuuuuuccuuucuuuuccuucuuuuuuuccucuuuucuugguggcuccaucuuagcccuagucacggcuagcugugaaagguccgugagccgcaugacugcagagagugccguaacuggccucucugcagaucauguucuag

SEQ ID NO: 67 - RNA, 1335 nt, Artificial Sequence
  Synthetic construct
aggaaacuuaagauuauuacaucaaaacaaaaagccgccaaugcuuuucaaucuccgcauccuccuuaacaacgccgcguuuagaaacggccacaacuucaugguccggaacuucagauguggccagccgcuucaaaacaagguccagcugaagggccgggaucuucugacccugaagaacuuuacuggcgaagagaucaaguacaugcucuggcucuccgcggacuugaaguuccgcauuaagcagaagggggaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucuggacacucuggccaaggaggcgucaauuccuauuaucaacggccuuagugaccucuaccauccgauucagauccuggccgauuaccucacccugcaagaacacuacagcucccugaagggucugacauuguccuggaucggcgacggcaacaacauucuccauuccaucaugauguccgccgcaaaauucggcaugcaucuucaagccgccacgccgaaggguuacgagcccgacgcuuccgugacuaagcucgccgagcaguacgcuaaggagaacggaaccaagcuucugcugacuaacgacccacuagaagcagcccacgggggcaacgugcuuauuacugacaccuggaucuccaugggccaggaagaagagaaaaagaagcggcugcaggcguuccagggauaucaggucaccaugaaaaccgccaaggucgcugccuccgacuggaccuuccugcacugccugccucgcaagccugaagaaguggacgacgagguguucuacucgccacggagccucguguuccccgaggccgagaauagaaaguggaccaucauggccgugauggugucacugcucaccgacuacagcccgcagcuucagaagcccaaguucuaggcuggagccucgguagccguuccuccugcccgcugggccucccaacgggcccuccuccccuccuugcaccggcccuuccuggucuuugaauaaagucugagugggcagcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 68 - RNA, 1335 nt, Artificial Sequence
  Synthetic construct
aggaaacuuaagauuauuacaucaaaacaaaaagccgccaaugcuguucaaccugcgcauccugcugaacaacgccgccuuccgcaacggccacaacuucauggugcgcaacuuccgcugcggccagccccugcagaacaaggugcagcugaagggccgcgaccugcugacccugaagaacuucaccggcgaggagaucaaguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggccuggcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugagcuggagccucgguagccguuccuccugcccgcugggccucccaacgggcccuccuccccuccuugcaccggcccuuccuggucuuugaauaaagucugagugggcagcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 69 - RNA, 1335 nt, Artificial Sequence
  Synthetic construct
aggaaacuuaagauuauuacaucaaaacaaaaagccgccaaugcuuuucaaccugagaauccucuugaacaaugcugcuuuucggaauggccacaacuuuaugguucggaacuuccguugcggccagccuuuacaaaacaagguccagcugaagggccgggauuugcucacacuaaagaacuuuacuggagaagagaucaaguacaugcuauggcugucggccgaccugaaguuccguaucaagcagaagggagaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucucgauaccuuggcaaaggaggcuuccauucccaucaucaacggccugagcgaccuguaccacccaauccaaauccuggcugacuaccugacccugcaagagcacuacagcagccugaagggucugacccugucauggauuggcgauggaaacaauauucugcacuccaucaugauguccgccgcgaaguucggaaugcaucugcaagccgccacuccaaaaggauacgaaccggaugcauccgugaccaaguuggcggaacaguacgcgaaggagaacggaaccaagcuccugcugacuaacgacccgcucgaggcugcgcauggggguaacgugcugauuacggacaccuggaucuccauggggcaggaggaagagaagaagaagagacugcaggcauuccagggguaccaggucaccaugaaaaccgcaaaaguggcagcuucggacuggacuuuccugcauugccugccgaggaagccggaggaagucgacgacgaaguguucuacucgccucggucccugguguuccccgaggccgaaaaccggaaguggaccaucauggccgugaugguguccuugcugacugacuauagcccgcagcugcagaagccuaaguucuaggcuggagccucgguagccguuccuccugcccgcugggccucccaacgggcccuccuccccuccuugcaccggcccuuccuggucuuugaauaaagucugagugggcagcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 70 - RNA, 1346 nt, Artificial Sequence
  Synthetic construct
aggaaacuuaagaauuauugguuaaagaaguauauuagugcuaauuucccuccguuuguccuagcuuuucucuucugucaaccccacacgccuuuggcacaaugcuuuucaaucuccgcauccuccuuaacaacgccgcguuuagaaacggccacaacuucaugguccggaacuucagauguggccagccgcuucaaaacaagguccagcugaagggccgggaucuucugacccugaagaacuuuacuggcgaagagaucaaguacaugcucuggcucuccgcggacuugaaguuccgcauuaagcagaagggggaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucuggacacucuggccaaggaggcgucaauuccuauuaucaacggccuuagugaccucuaccauccgauucagauccuggccgauuaccucacccugcaagaacacuacagcucccugaagggucugacauuguccuggaucggcgacggcaacaacauucuccauuccaucaugauguccgccgcaaaauucggcaugcaucuucaagccgccacgccgaaggguuacgagcccgacgcuuccgugacuaagcucgccgagcaguacgcuaaggagaacggaaccaagcuucugcugacuaacgacccacuagaagcagcccacgggggcaacgugcuuauuacugacaccuggaucuccaugggccaggaagaagagaaaaagaagcggcugcaggcguuccagggauaucaggucaccaugaaaaccgccaaggucgcugccuccgacuggaccuuccugcacugccugccucgcaagccugaagaaguggacgacgagguguucuacucgccacggagccucguguuccccgaggccgagaauagaaaguggaccaucauggccgugauggugucacugcucaccgacuacagcccgcagcuucagaagcccaaguucuagcucgagacacaucacaaccacaaccuucucaggcuacccugagaaaaaaagacaugaagacucaggacucaucuuuucuguugguguaaaaucaacacccuaaggaacacaaauuucuuuaaacauuugacuucuugucucugugcugcaauuaauaaaaaauggaaagaaucuaucuag

SEQ ID NO: 71 - RNA, 1346 nt, Artificial Sequence
  Synthetic construct
aggaaacuuaagaauuauugguuaaagaaguauauuagugcuaauuucccuccguuuguccuagcuuuucucuucugucaaccccacacgccuuuggcacaaugcuguucaaccugcgcauccugcugaacaacgccgccuuccgcaacggccacaacuucauggugcgcaacuuccgcugcggccagccccugcagaacaaggugcagcugaagggccgcgaccugcugacccugaagaacuucaccggcgaggagaucaaguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggccuggcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugacucgagacacaucacaaccacaaccuucucaggcuacccugagaaaaaaagacaugaagacucaggacucaucuuuucuguugguguaaaaucaacacccuaaggaacacaaauuucuuuaaacauuugacuucuugucucugugcugcaauuaauaaaaaauggaaagaaucuaucuag

SEQ ID NO: 72 - RNA, 1346 nt, Artificial Sequence
  Synthetic construct
aggaaacuuaagaauuauugguuaaagaaguauauuagugcuaauuucccuccguuuguccuagcuuuucucuucugucaaccccacacgccuuuggcacaaugcuuuucaaccugagaauccucuugaacaaugcugcuuuucggaauggccacaacuuuaugguucggaacuuccguugcggccagccuuuacaaaacaagguccagcugaagggccgggauuugcucacacuaaagaacuuuacuggagaagagaucaaguacaugcuauggcugucggccgaccugaaguuccguaucaagcagaagggagaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucucgauaccuuggcaaaggaggcuuccauucccaucaucaacggccugagcgaccuguaccacccaauccaaauccuggcugacuaccugacccugcaagagcacuacagcagccugaagggucugacccugucauggauuggcgauggaaacaauauucugcacuccaucaugauguccgccgcgaaguucggaaugcaucugcaagccgccacuccaaaaggauacgaaccggaugcauccgugaccaaguuggcggaacaguacgcgaaggagaacggaaccaagcuccugcugacuaacgacccgcucgaggcugcgcauggggguaacgugcugauuacggacaccuggaucuccauggggcaggaggaagagaagaagaagagacugcaggcauuccagggguaccaggucaccaugaaaaccgcaaaaguggcagcuucggacuggacuuuccugcauugccugccgaggaagccggaggaagucgacgacgaaguguucuacucgccucggucccugguguuccccgaggccgaaaaccggaaguggaccaucauggccgugaugguguccuugcugacugacuauagcccgcagcugcagaagccuaaguucuagcucgagacacaucacaaccacaaccuucucaggcuacccugagaaaaaaagacaugaagacucaggacucaucuuuucuguugguguaaaaucaacacccuaaggaacacaaauuucuuuaaacauuugacuucuugucucugugcugcaauuaauaaaaaauggaaagaaucuaucuag

SEQ ID NO: 73 - RNA, 1222 nt, Artificial Sequence
  Synthetic construct
auuauuacaucaaaacaaaaagccgccaccaugcuguucaaccugcgcauccugcugaacaacgccgccuuccgcaacggccacaacuucauggugcgcaacuuccgcugcggccagccccugcagaacaaggugcagcugaagggccgcgaccugcugacccugaagaacuucaccggcgaggagaucaaguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggcuucgcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugaggucucuaguaaugagcuggagccucgguagccguuccuccugcccgcugggccucccaacgggcccuccuccccuccuugcaccggcccuuccuggucuuugaauaaagucugagugggcaucuag

SEQ ID NO: 74 - RNA, 1346 nt, Artificial Sequence
  Synthetic construct
aggaaacuuaagaauuauugguuaaagaaguauauuagugcuaauuucccuccguuuguccuagcuuuucucuucugucaaccccacacgccuuuggcacaaugcuguucaaccugcgcauccugcugaacaacgccgccuuccgcaacggccacaacuucauggugcgcaacuuccgcugcggccagccccugcagaacaaggugcagcugaagggccgcgaccugcugacccugaagaacuucaccggcgaggagaucaaguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggcuucgcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugacucgagacacaucacaaccacaaccuucucaggcuacccugagaaaaaaagacaugaagacucaggacucaucuuuucuguugguguaaaaucaacacccuaaggaacacaaauuucuuuaaacauuugacuucuugucucugugcugcaauuaauaaaaaauggaaagaaucuaucuag

SEQ ID NO: 75 - RNA, 1368 nt, Artificial Sequence
  Synthetic construct
aggaaacuuaagauuauuacaucaaaacaaaaagccgccaaugcuguucaaccugcgcauccugcugaacaacgccgccuuccgcaacggccacaacuucauggugcgcaacuuccgcugcggccagccccugcagaacaaggugcagcugaagggccgcgaccugcugacccugaagaacuucaccggcgaggagaucaaguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggcuucgcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugaacgccgaagccugcagccaugcgaccccacgccaccccgugccuccugccuccgcgcagccugcagcgggagacccuguccccgccccagccguccuccugggguggacccuaguuuaauaaagauucaccaaguuucacgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 76 - RNA, 1496 nt, Artificial Sequence
  Synthetic construct
cuuaagggggcgcugccuacggagguggcagccaucuccuucucggcaucaagcuuaccauggugccccaggcccugcucuuggucccgcugcugguguucccccucugcuucggcaaguuccccaucuacaccauccccgacaagcuggggccguggagccccaucgacauccaccaccuguccugccccaacaaccucguggucgaggacgagggcugcaccaaccugagcggguucuccuacaugcuguucaaccugcgcauccugcugaacaacgccgccuuccgcaacggccacaacuucauggugcgcaacuuccgcugcggccagccccugcagaacaaggugcagcugaagggccgcgaccugcugacccugaagaacuucaccggcgaggagaucaaguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggcuucgcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugaauaagugaaugcaaggcuggccggaagcccuugccugaaagcaagauuucagccuggaagagggcaaaguggacgggaguggacaggaguggaugcgauaagaugugguuugaagcugaugggugccagcccugcauugcugagucaaucaauaaagagcuuucuuuugacccauucuagaucuag

SEQ ID NO: 77 - RNA, 1380 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugggcgucuucaaccugcggauccugcugaacaacgccgccuuccggaacggccacaacuucaugguccgcaacuucagaugcggccagccccugcagaacaaggugcagcugaagggccgggaccugcugacccugaagaacuucaccggcgaagagaucaaguacaugcuguggcugagcgccgaccugaaguuccggaucaagcagaagggcgaguaccugccccugcugcaaggcaagagccugggcaugaucuucgagaagcggagcacccggacccggcugagcaccgagacaggcuuugcccugcugggaggccaccccugcuuucugaccacccaggacauccaccugggcgugaacgagagccugaccgacaccgccagagugcugagcagcauggccgacgccgugcuggcccggguguacaagcagagcgaccuggacacccuggccaaagaggccagcauccccaucaucaacggccugagcgaccuguaccaccccauccagauccuggccgacuaccugacccugcaggaacacuacagcucccugaagggccugacccugagcuggaucggcgacggcaacaacauccugcacagcaucaugaugagcgccgccaaguucggcaugcaucugcaggccgccacccccaagggcuacgagccugaugccagcgugaccaagcuggccgagcaguacgccaaagagaacggcaccaagcugcugcugaccaacgacccccuggaagccgcccacggcggcaacgugcugaucaccgacaccuggaucagcaugggccaggaagaggaaaagaagaagcggcugcaggccuuccagggcuaccaggucacaaugaagaccgccaagguggccgccagcgacuggaccuuccugcacugccugccccggaagcccgaagagguggacgacgagguguucuacagcccccggucccugguguuccccgaggccgagaaccggaaguggaccauuauggccgugauggugucccugcugaccgacuacuccccccagcugcagaagcccaaguucuagauaagugaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 78 - RNA, 1380 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugggcgucuucaaccugcggauccugcugaacaacgccgccuuccggaacggccacaacuucaugguccgcaacuucagaugcggccagccccugcagaacagggugcagcugaagggccgggaccugcugacccugaagaacuucaccggcgaagagaucagguacaugcuguggcugagcgccgaccugaaguuccggaucaagcagaagggcgaguaccugccccugcugcaaggcaagagccugggcaugaucuucgagaagcggagcacccggacccggcugagcaccgagacaggcuuugcccugcugggaggccaccccugcuuucugaccacccaggacauccaccugggcgugaacgagagccugaccgacaccgccagagugcugagcagcauggccgacgccgugcuggcccggguguacaagcagagcgaccuggacacccuggccaaagaggccagcauccccaucaucaacggccugagcgaccuguaccaccccauccagauccuggccgacuaccugacccugcaggaacacuacagcucccugaagggccugacccugagcuggaucggcgacggcaacaacauccugcacagcaucaugaugagcgccgccaaguucggcaugcaucugcaggccgccacccccaagggcuacgagccugaugccagcgugaccaagcuggccgagcaguacgccaaagagaacggcaccaagcugcugcugaccaacgacccccuggaagccgcccacggcggcaacgugcugaucaccgacaccuggaucagcaugggccaggaagaggaaaagaagaagcggcugcaggccuuccagggcuaccaggucacaaugaagaccgccaagguggccgccagcgacuggaccuuccugcacugccugccccggaagcccgaagagguggacgacgagguguucuacagcccccggucccugguguuccccgaggccgagaaccggaaguggaccauuauggccgugauggugucccugcugaccgacuacuccccccagcugcagaagcccaaguucuagauaagugaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 79 - RNA, 1380 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuggucuucaaccugcggauccugcugaacaacgccgccuuccggaacggccacaacuucaugguccgcaacuucagaugcggccagccccugcagaacagggugcagcugaagggccgggaccugcugacccugaagaacuucaccggcgaagagaucagguacaugcuguggcugagcgccgaccugaaguuccggaucaagcagaagggcgaguaccugccccugcugcaaggcaagagccugggcaugaucuucgagaagcggagcacccggacccggcugagcaccgagacaggcuuugcccugcugggaggccaccccugcuuucugaccacccaggacauccaccugggcgugaacgagagccugaccgacaccgccagagugcugagcagcauggccgacgccgugcuggcccggguguacaagcagagcgaccuggacacccuggccaaagaggccagcauccccaucaucaacggccugagcgaccuguaccaccccauccagauccuggccgacuaccugacccugcaggaacacuacagcucccugaagggccugacccugagcuggaucggcgacggcaacaacauccugcacagcaucaugaugagcgccgccaaguucggcaugcaucugcaggccgccacccccaagggcuacgagccugaugccagcgugaccaagcuggccgagcaguacgccaaagagaacggcaccaagcugcugcugaccaacgacccccuggaagccgcccacggcggcaacgugcugaucaccgacaccuggaucagcaugggccaggaagaggaaaagaagaagcggcugcaggccuuccagggcuaccaggucacaaugaagaccgccaagguggccgccagcgacuggaccuuccugcacugccugccccggaagcccgaagagguggacgacgagguguucuacagcccccggucccugguguuccccgaggccgagaaccggaaguggaccauuauggccgugauggugucccugcugaccgacuacuccccccagcugcagaagcccaaguucuagauaagugaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 80 - RNA, 1377 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuguucaaccugaggauccugcugaacaacgcagcuuucaggaacggccacaacuucauggugaggaacuuccggugcggccagccccugcagaacaaggugcagcugaagggcagggaccugcugacccugaagaacuucaccggagaggagaucaaguacaugcuguggcugagcgcagaccugaaguucaggaucaagcagaagggagaguaccugccccugcugcaggggaagucccugggcaugaucuucgagaagaggaguaccaggaccaggcugagcaccgaaaccggcuucgcccugcugggaggacaccccugcuuccugaccacccaggacauccaccugggcgugaacgagagucugaccgacaccgccagggugcugucuagcauggccgacgccgugcuggccaggguguacaagcagucagaccuggacacccuggcuaaggaggccagcauccccaucaucaacggccugagcgaccuguaccaccccauccagauccuggcugacuaccugacccugcaggagcacuacagcucucugaagggccugacccugagcuggaucggcgacgggaacaacauccugcacagcaucaugaugagcgccgccaaguucggcaugcaccugcaggccgcuacccccaaggguuacgagcccgacgccagcgugaccaagcuggcagagcaguacgccaaggagaacggcaccaagcugcugcugaccaacgacccccuggaggccgcccacggaggcaacgugcugaucaccgacaccuggaucagcaugggacaggaggaggagaagaagaagcggcugcaggcuuuccaggguuaccaggugaccaugaagaccgccaagguggcugccagcgacuggaccuuccugcacugccugcccaggaagcccgaggagguggacgacgagguguucuacucucccaggagccugguguuccccgaggccgagaacaggaaguggaccaucauggcugugauggugucccugcugaccgacuacagcccccagcugcagaagcccaaguucugaauaagugaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 81 - RNA, 1377 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuguucaaccugaggauccugcugaacaacgcagcuuucaggaacggccacaacuucauggugaggaacuuccggugcggccagccccugcagaacaaggugcagcugaagggcagggaccugcugacccugaagaacuucaccggagaggagaucaaguacaugcuguggcugagcgcagaccugaaguucaggaucaagcagaagggagaguaccugccccugcugcaggggaagucccugggcaugaucuucgagaagaggaguaccaggaccaggcugagcaccgaaaccggcuucgcccugcugggaggacaccccugcuuccugacgacccaggacauccaccucggagugaacgaaucccucaccgauaccgcccggguguuaucaagcauggcagaugccgugcuggccaggguguacaaacaguccgaucuggacacucuggcuaaggaggccagcauccccaucaucaacggccugagcgaccuguaccaccccauccagauccuggcugacuaccugacccugcaggagcacuacagcucucugaagggccugacccugagcuggaucggcgacgggaacaacauccugcacuccaucaugauguccgccgcgaaguucggaaugcaucugcaagccgccacgccaaaaggauacgaaccggaugcgcccgugacaaaguuggcggaacaguacgcuaaggagaacggaaccaagcugcugcugaccaacgacccccuggaggccgcccacggaggcaacgugcugaucaccgacaccuggaucagcaugggacaggaggaggagaagaagaagcggcugcaggcuuuccaggguuaccaggugaccaugaagaccgccaagguggcugccagcgacuggaccuuccugcacugccugcccaggaagcccgaggagguggacgacgagguguucuacucucccaggagccugguguuccccgaggccgagaacaggaaguggaccaucauggcugugauggugucccugcugaccgacuacagcccccagcugcagaagcccaaguucugaauaagugaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 82 - RNA, 1477 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuguucaaccugcgcauccugcugaacaacgccgccuuccgcaacggccacaacuucauggugcgcaacuuccgcugcggccagccccugcagaacaaggugcagcugaagggccgcgaccugcugacccugaagaacuucaccggcgaggagaucaaguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggcuucgcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugaauaagugaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 83 - RNA, 1371 nt, Artificial Sequence
  Synthetic construct
aggaaacuuaagucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaugcuguucaaccugcgcauccugcugaacaacgccgccuuccgcaacggccacaacuucauggugcgcaacuuccgcugcggccagccccugcagaacaaggugcagcugaagggccgcgaccugcugacccugaagaacuucaccggcgaggagaucaaguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggcuucgcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugacuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 84 - RNA, 1371 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuuuucaacuugagaauccugcugaacaacgccgccuuucgcaacggucacaauuuuauggucagaaacuucagaugcggacagccccuccaaaacaagguccagcugaagggccgcgaucuccucacccugaagaacuucacgggggaggagaucaaguacaugcuguggcucuccgcugaccugaaguucaggaucaagcagaagggagaauaucugccgcugcugcaagggaagucccuggggaugauuuucgagaagcggagcacccggacucggcucuccacugaaacugguuucgcccuucugggcggucaccccugcuuccugaccacucaagacauucaccucggagugaacgaguccuugacugacaccgcccgggugcugucgagcauggcagacgccgugcuagcccgcguguacaagcagucagaccucgauacccuggccaaggaggcuucgaucccgaucaucaacggguuguccgaccuguaccacccgauucagauucucgccgacuaccucacccugcaagagcauuacagcucccugaaggggcuuacccuguccuggauuggcgacggaaacaacauccugcacuccauuaugaugucggcggccaaguucggcaugcaccuccaagccgcgaccccuaaggguuacgaaccagacgcgucagugacuaagcuggccgaacaguacgcaaaggaaaauggcacgaagcugcuccugaccaacgauccguuggaagccgcccauggcggaaaugugcucaucaccgacaccuggaucucgaugggacaggaggaagagaagaagaagcggcugcaggcguuccagggcuaccaggucaccaugaaaacugccaagguggccgccagcgacuggaccuuccugcacugccuuccgcgcaagccugaggagguggacgaugaaguguucuacucuccacggucccugguguuccccgaggcggagaaccgcaaauggaccaucauggcugugauggucagccugcugaccgauuacagcccucaguugcaaaagccgaaguuuugauaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 85 - RNA, 1471 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuguucaaccuccgcauccuccucaacaacgccgcauucagaaacgggcacaacuucauggucagaaacuuccgcugcgggcaaccccuacaaaacaagguccagcucaaggggcgggaccuccugacccugaagaacuucaccggcgaagagaucaaguacaugcuguggcucuccgccgaccugaaguuccgcaucaagcagaagggagaguaccucccgcugcugcaagggaagucgcuggggaugaucuucgagaagcggucaaccagaacccggcugucaaccgaaaccggguucgcacugcuggggggacacccgugcuuccugaccacccaagacauccaccugggagugaacgaaucgcugaccgacaccgcccgcgugcugagcucaauggcggacgccgugcuggcccgcguguacaagcaguccgaccuggacacccuggccaaggaagcguccaucccgaucaucaacggacuguccgaccuguaccacccgauccagauccuggcagacuaccugacccugcaagaacacuacagcucccugaagggccugacccugucauggaucggggacgggaacaacauccugcacuccauaaugaugucagccgccaaguucggaaugcaccuccaagccgcaaccccgaagggcuacgaaccggacgcaucagugaccaaacuggccgagcaguacgccaaggaaaacggcaccaagcuccugcugaccaacgacccgcuggaggccgcacacggggggaacgugcugaucaccgacaccuggaucuccaugggacaggaggaggaaaagaagaagcggcugcaggcguuccagggguaccaggucaccaugaaaaccgcgaaggucgcggcaucagacuggaccuuccugcacugccugccccggaagccggaagagguggacgacgagguguucuacucgccgcgcucgcugguguuccccgaggcggagaacaggaaguggaccaucauggcggugauggucagccuccugaccgacuacucgccgcagcugcagaagccgaaguucugauaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 86 - RNA, 1771 nt, Artificial Sequence
  Synthetic construct
cucccuccccccccccuaacguuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacguguguuuagucgagguuaaaaaacgucuaggccccccgaaccacggggacgugguuuuccuuugaaaaacacgaugauaauaugcuuuucaaucuccgcauccuccuuaacaacgccgcguuuagaaacggccacaacuucaugguccggaacuucagauguggccagccgcuucaaaacaagguccagcugaagggccgggaucuucugacccugaagaacuuuacuggcgaagagaucaaguacaugcucuggcucuccgcggacuugaaguuccgcauuaagcagaagggggaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucuggacacucuggccaaggaggcgucaauuccuauuaucaacggccuuagugaccucuaccauccgauucagauccuggccgauuaccucacccugcaagaacacuacagcucccugaagggucugacauuguccuggaucggcgacggcaacaacauucuccauuccaucaugauguccgccgcaaaauucggcaugcaucuucaagccgccacgccgaaggguuacgagcccgacgcuuccgugacuaagcucgccgagcaguacgcuaaggagaacggaaccaagcuucugcugacuaacgacccacuagaagcagcccacgggggcaacgugcuuauuacugacaccuggaucuccaugggccaggaagaagagaaaaagaagcggcugcaggcguuccagggauaucaggucaccaugaaaaccgccaaggucgcugccuccgacuggaccuuccugcacugccugccucgcaagccugaagaaguggacgacgagguguucuacucgccacggagccucguguuccccgaggccgagaauagaaaguggaccaucauggccgugauggugucacugcucaccgacuacagcccgcagcuucagaagcccaaguucugaauaaguagauagugcagucacuggcacaacgcguugcccgguaagccaaucggguauacacggucgucauacugcagacaggguucuucuacuuugcaagauagucuagaguaguaaaauaaauaguauaagucuag

SEQ ID NO: 87 - RNA, 1771 nt, Artificial Sequence
  Synthetic construct
cucccuccccccccccuaacguuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacguguguuuagucgagguuaaaaaacgucuaggccccccgaaccacggggacgugguuuuccuuugaaaaacacgaugauaauaugcuuuucaaccugagaauccucuugaacaaugcugcuuuucggaauggccacaacuuuaugguucggaacuuccguugcggccagccuuuacaaaacaagguccagcugaagggccgggauuugcucacacuaaagaacuuuacuggagaagagaucaaguacaugcuauggcugucggccgaccugaaguuccguaucaagcagaagggagaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucucgauaccuuggcaaaggaggcuuccauucccaucaucaacggccugagcgaccuguaccacccaauccaaauccuggcugacuaccugacccugcaagagcacuacagcagccugaagggucugacccugucauggauuggcgauggaaacaauauucugcacuccaucaugauguccgccgcgaaguucggaaugcaucugcaagccgccacuccaaaaggauacgaaccggaugcauccgugaccaaguuggcggaacaguacgcgaaggagaacggaaccaagcuccugcugacuaacgacccgcucgaggcugcgcauggggguaacgugcugauuacggacaccuggaucuccauggggcaggaggaagagaagaagaagagacugcaggcauuccagggguaccaggucaccaugaaaaccgcaaaaguggcagcuucggacuggacuuuccugcauugccugccgaggaagccggaggaagucgacgacgaaguguucuacucgccucggucccugguguuccccgaggccgaaaaccggaaguggaccaucauggccgugaugguguccuugcugacugacuauagcccgcagcugcagaagccuaaguucugaauaaguagauagugcagucacuggcacaacgcguugcccgguaagccaaucggguauacacggucgucauacugcagacaggguucuucuacuuugcaagauagucuagaguaguaaaauaaauaguauaagucuag

SEQ ID NO: 88 - RNA, 1771 nt, Artificial Sequence
  Synthetic construct
cucccuccccccccccuaacguuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacguguguuuagucgagguuaaaaaacgucuaggccccccgaaccacggggacgugguuuuccuuugaaaaacacgaugauaauaugcuguucaaccugcgcauccugcugaacaacgccgccuuccgcaacggccacaacuucauggugcgcaacuuccgcugcggccagccccugcagaacaaggugcagcugaagggccgcgaccugcugacccugaagaacuucaccggcgaggagaucaaguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggccuggcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugaauaaguagauagugcagucacuggcacaacgcguugcccgguaagccaaucggguauacacggucgucauacugcagacaggguucuucuacuuugcaagauagucuagaguaguaaaauaaauaguauaagucuag

SEQ ID NO: 89 - RNA, 1368 nt, Artificial Sequence; identical to SEQ ID NO: 34
  Synthetic construct

SEQ ID NO: 90 - RNA, 1368 nt, Artificial Sequence; identical to SEQ ID NO: 34
  Synthetic construct

SEQ ID NO: 91 - RNA, 1368 nt, Artificial Sequence; identical to SEQ ID NO: 34
  Synthetic construct

SEQ ID NO: 92 - RNA, 1368 nt, Artificial Sequence; identical to SEQ ID NO: 34
  Synthetic construct

SEQ ID NO: 93 - RNA, 1368 nt, Artificial Sequence; identical to SEQ ID NO: 34
  Synthetic construct

SEQ ID NO: 94 - RNA, 1371 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugguguucaaccuccgcauccuccucaacaacgccgcauucagaaacgggcacaacuucauggucagaaacuuccgcugcgggcaaccccuacaaaacaagguccagcucaaggggcgggaccuccugacccugaagaacuucaccggcgaagagaucaaguacaugcuguggcucuccgccgaccugaaguuccgcaucaagcagaagggagaguaccucccgcugcugcaagggaagucgcuggggaugaucuucgagaagcggucaaccagaacccggcugucaaccgaaaccggguucgcacugcuggggggacacccgugcuuccugaccacccaagacauccaccugggagugaacgaaucgcugaccgacaccgcccgcgugcugagcucaauggcggacgccgugcuggcccgcguguacaagcaguccgaccuggacacccuggccaaggaagcguccaucccgaucaucaacggacuguccgaccuguaccacccgauccagauccuggcagacuaccugacccugcaagaacacuacagcucccugaagggccugacccugucauggaucggggacgggaacaacauccugcacuccauaaugaugucagccgccaaguucggaaugcaccuccaagccgcaaccccgaagggcuacgaaccggacgcaucagugaccaaacuggccgagcaguacgccaaggaaaacggcaccaagcuccugcugaccaacgacccgcuggaggccgcacacggggggaacgugcugaucaccgacaccuggaucuccaugggacaggaggaggaaaagaagaagcggcugcaggcguuccagggguaccaggucaccaugaaaaccgcgaaggucgcggcaucagacuggaccuuccugcacugccugccccggaagccggaagagguggacgacgagguguucuacucgccgcgcucgcugguguuccccgaggcggagaacaggaaguggaccaucauggcggugauggucagccuccugaccgacuacucgccgcagcugcagaagccgaaguucugauaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 95 - RNA, 1371 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugguguucaaccuccgcauccuccucaacaacgccgcauucagaaacgggcacaacuucauggucagaaacuuccgcugcgggcaaccccuacaaaaccggguccagcucaaggggcgggaccuccugacccugaagaacuucaccggcgaagagaucaaguacaugcuguggcucuccgccgaccugaaguuccgcaucaagcagaagggagaguaccucccgcugcugcaagggaagucgcuggggaugaucuucgagaagcggucaaccagaacccggcugucaaccgaaaccggguucgcacugcuggggggacacccgugcuuccugaccacccaagacauccaccugggagugaacgaaucgcugaccgacaccgcccgcgugcugagcucaauggcggacgccgugcuggcccgcguguacaagcaguccgaccuggacacccuggccaaggaagcguccaucccgaucaucaacggacuguccgaccuguaccacccgauccagauccuggcagacuaccugacccugcaagaacacuacagcucccugaagggccugacccugucauggaucggggacgggaacaacauccugcacuccauaaugaugucagccgccaaguucggaaugcaccuccaagccgcaaccccgaagggcuacgaaccggacgcaucagugaccaaacuggccgagcaguacgccaaggaaaacggcaccaagcuccugcugaccaacgacccgcuggaggccgcacacggggggaacgugcugaucaccgacaccuggaucuccaugggacaggaggaggaaaagaagaagcggcugcaggcguuccagggguaccaggucaccaugaaaaccgcgaaggucgcggcaucagacuggaccuuccugcacugccugccccggaagccggaagagguggacgacgagguguucuacucgccgcgcucgcugguguuccccgaggcggagaacaggaaguggaccaucauggcggugauggucagccuccugaccgacuacucgccgcagcugcagaagccgaaguucugauaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 96 - RNA, 1371 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugguguucaaccuccgcauccuccucaacaacgccgcauucagaaacgggcacaacuucauggucagaaacuuccgcugcgggcaaccccuacaaaaccggguccagcucaaggggcgggaccuccugacccugaagaacuucaccggcgaagagauccgguacaugcuguggcucuccgccgaccugaaguuccgcaucaagcagaagggagaguaccucccgcugcugcaagggaagucgcuggggaugaucuucgagaagcggucaaccagaacccggcugucaaccgaaaccggguucgcacugcuggggggacacccgugcuuccugaccacccaagacauccaccugggagugaacgaaucgcugaccgacaccgcccgcgugcugagcucaauggcggacgccgugcuggcccgcguguacaagcaguccgaccuggacacccuggccaaggaagcguccaucccgaucaucaacggacuguccgaccuguaccacccgauccagauccuggcagacuaccugacccugcaagaacacuacagcucccugaagggccugacccugucauggaucggggacgggaacaacauccugcacuccauaaugaugucagccgccaaguucggaaugcaccuccaagccgcaaccccgaagggcuacgaaccggacgcaucagugaccaaacuggccgagcaguacgccaaggaaaacggcaccaagcuccugcugaccaacgacccgcuggaggccgcacacggggggaacgugcugaucaccgacaccuggaucuccaugggacaggaggaggaaaagaagaagcggcugcaggcguuccagggguaccaggucaccaugaaaaccgcgaaggucgcggcaucagacuggaccuuccugcacugccugccccggaagccggaagagguggacgacgagguguucuacucgccgcgcucgcugguguuccccgaggcggagaacaggaaguggaccaucauggcggugauggucagccuccugaccgacuacucgccgcagcugcagaagccgaaguucugauaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 97 - RNA, 1371 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuggucaaccuccgcauccuccucaacaacgccgcauucagaaacgggcacaacuucauggucagaaacuuccgcugcgggcaaccccuacaaaacaagguccagcucaaggggcgggaccuccugacccugaagaacuucaccggcgaagagaucaaguacaugcuguggcucuccgccgaccugaaguuccgcaucaagcagaagggagaguaccucccgcugcugcaagggaagucgcuggggaugaucuucgagaagcggucaaccagaacccggcugucaaccgaaaccggguucgcacugcuggggggacacccgugcuuccugaccacccaagacauccaccugggagugaacgaaucgcugaccgacaccgcccgcgugcugagcucaauggcggacgccgugcuggcccgcguguacaagcaguccgaccuggacacccuggccaaggaagcguccaucccgaucaucaacggacuguccgaccuguaccacccgauccagauccuggcagacuaccugacccugcaagaacacuacagcucccugaagggccugacccugucauggaucggggacgggaacaacauccugcacuccauaaugaugucagccgccaaguucggaaugcaccuccaagccgcaaccccgaagggcuacgaaccggacgcaucagugaccaaacuggccgagcaguacgccaaggaaaacggcaccaagcuccugcugaccaacgacccgcuggaggccgcacacggggggaacgugcugaucaccgacaccuggaucuccaugggacaggaggaggaaaagaagaagcggcugcaggcguuccagggguaccaggucaccaugaaaaccgcgaaggucgcggcaucagacuggaccuuccugcacugccugccccggaagccggaagagguggacgacgagguguucuacucgccgcgcucgcugguguuccccgaggcggagaacaggaaguggaccaucauggcggugauggucagccuccugaccgacuacucgccgcagcugcagaagccgaaguucugauaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 98 - RNA, 1471 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuggucaaccuccgcauccuccucaacaacgccgcauucagaaacgggcacaacuucauggucagaaacuuccgcugcgggcaaccccuacaaaaccggguccagcucaaggggcgggaccuccugacccugaagaacuucaccggcgaagagaucaaguacaugcuguggcucuccgccgaccugaaguuccgcaucaagcagaagggagaguaccucccgcugcugcaagggaagucgcuggggaugaucuucgagaagcggucaaccagaacccggcugucaaccgaaaccggguucgcacugcuggggggacacccgugcuuccugaccacccaagacauccaccugggagugaacgaaucgcugaccgacaccgcccgcgugcugagcucaauggcggacgccgugcuggcccgcguguacaagcaguccgaccuggacacccuggccaaggaagcguccaucccgaucaucaacggacuguccgaccuguaccacccgauccagauccuggcagacuaccugacccugcaagaacacuacagcucccugaagggccugacccugucauggaucggggacgggaacaacauccugcacuccauaaugaugucagccgccaaguucggaaugcaccuccaagccgcaaccccgaagggcuacgaaccggacgcaucagugaccaaacuggccgagcaguacgccaaggaaaacggcaccaagcuccugcugaccaacgacccgcuggaggccgcacacggggggaacgugcugaucaccgacaccuggaucuccaugggacaggaggaggaaaagaagaagcggcugcaggcguuccagggguaccaggucaccaugaaaaccgcgaaggucgcggcaucagacuggaccuuccugcacugccugccccggaagccggaagagguggacgacgagguguucuacucgccgcgcucgcugguguuccccgaggcggagaacaggaaguggaccaucauggcggugauggucagccuccugaccgacuacucgccgcagcugcagaagccgaaguucugauaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 99 - RNA, 1371 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuggucaaccuccgcauccuccucaacaacgccgcauucagaaacgggcacaacuucauggucagaaacuuccgcugcgggcaaccccuacaaaaccggguccagcucaaggggcgggaccuccugacccugaagaacuucaccggcgaagagauccgguacaugcuguggcucuccgccgaccugaaguuccgcaucaagcagaagggagaguaccucccgcugcugcaagggaagucgcuggggaugaucuucgagaagcggucaaccagaacccggcugucaaccgaaaccggguucgcacugcuggggggacacccgugcuuccugaccacccaagacauccaccugggagugaacgaaucgcugaccgacaccgcccgcgugcugagcucaauggcggacgccgugcuggcccgcguguacaagcaguccgaccuggacacccuggccaaggaagcguccaucccgaucaucaacggacuguccgaccuguaccacccgauccagauccuggcagacuaccugacccugcaagaacacuacagcucccugaagggccugacccugucauggaucggggacgggaacaacauccugcacuccauaaugaugucagccgccaaguucggaaugcaccuccaagccgcaaccccgaagggcuacgaaccggacgcaucagugaccaaacuggccgagcaguacgccaaggaaaacggcaccaagcuccugcugaccaacgacccgcuggaggccgcacacggggggaacgugcugaucaccgacaccuggaucuccaugggacaggaggaggaaaagaagaagcggcugcaggcguuccagggguaccaggucaccaugaaaaccgcgaaggucgcggcaucagacuggaccuuccugcacugccugccccggaagccggaagagguggacgacgagguguucuacucgccgcgcucgcugguguuccccgaggcggagaacaggaaguggaccaucauggcggugauggucagccuccugaccgacuacucgccgcagcugcagaagccgaaguucugauaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 100 - RNA, 1471 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuggucaaccugcgcauccugcugaacaacgccgccuuccgcaacggccacaacuucauggugcgcaacuuccgcugcggccagccccugcagaacaaggugcagcugaagggccgcgaccugcugacccugaagaacuucaccggcgaggagaucaaguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggcuucgcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugauaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 101 - RNA, 1371 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuggucaaccugcgcauccugcugaacaacgccgccuuccgcaacggccacaacuucauggugcgcaacuuccgcugcggccagccccugcagaaccgggugcagcugaagggccgcgaccugcugacccugaagaacuucaccggcgaggagaucaaguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggcuucgcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugauaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 102 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuugucaaucuccgcauccuccuuaacaacgccgcguuuagaaacggccacaacuucaugguccggaacuucagauguggccagccgcuucaaaacaagguccagcugaagggccgggaucuucugacccugaagaacuuuacuggcgaagagaucaaguacaugcucuggcucuccgcggacuugaaguuccgcauuaagcagaagggggaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucuggacacucuggccaaggaggcgucaauuccuauuaucaacggccuuagugaccucuaccauccgauucagauccuggccgauuaccucacccugcaagaacacuacagcucccugaagggucugacauuguccuggaucggcgacggcaacaacauucuccauuccaucaugauguccgccgcaaaauucggcaugcaucuucaagccgccacgccgaaggguuacgagcccgacgcuuccgugacuaagcucgccgagcaguacgcuaaggagaacggaaccaagcuucugcugacuaacgacccacuagaagcagcccacgggggcaacgugcuuauuacugacaccuggaucuccaugggccaggaagaagagaaaaagaagcggcugcaggcguuccagggauaucaggucaccaugaaaaccgccaaggucgcugccuccgacuggaccuuccugcacugccugccucgcaagccugaagaaguggacgacgagguguucuacucgccacggagccucguguuccccgaggccgagaauagaaaguggaccaucauggccgugauggugucacugcucaccgacuacagcccgcagcuucagaagcccaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 103 - RNA, 1368 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugcuugucaaucuccgcauccuccuuaacaacgccgcguuuagaaacggccacaacuucaugguccggaacuucagauguggccagccgcuucaaaaccggguccagcugaagggccgggaucuucugacccugaagaacuuuacuggcgaagagaucaaguacaugcucuggcucuccgcggacuugaaguuccgcauuaagcagaagggggaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucuggacacucuggccaaggaggcgucaauuccuauuaucaacggccuuagugaccucuaccauccgauucagauccuggccgauuaccucacccugcaagaacacuacagcucccugaagggucugacauuguccuggaucggcgacggcaacaacauucuccauuccaucaugauguccgccgcaaaauucggcaugcaucuucaagccgccacgccgaaggguuacgagcccgacgcuuccgugacuaagcucgccgagcaguacgcuaaggagaacggaaccaagcuucugcugacuaacgacccacuagaagcagcccacgggggcaacgugcuuauuacugacaccuggaucuccaugggccaggaagaagagaaaaagaagcggcugcaggcguuccagggauaucaggucaccaugaaaaccgccaaggucgcugccuccgacuggaccuuccugcacugccugccucgcaagccugaagaaguggacgacgagguguucuacucgccacggagccucguguuccccgaggccgagaauagaaaguggaccaucauggccgugauggugucacugcucaccgacuacagcccgcagcuucagaagcccaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 104 - RNA, 1371 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugggccuugucaaucuccgcauccuccuuaacaacgccgcguuuagaaacggccacaacuucaugguccggaacuucagauguggccagccgcuucaaaacaagguccagcugaagggccgggaucuucugacccugaagaacuuuacuggcgaagagaucaaguacaugcucuggcucuccgcggacuugaaguuccgcauuaagcagaagggggaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucuggacacucuggccaaggaggcgucaauuccuauuaucaacggccuuagugaccucuaccauccgauucagauccuggccgauuaccucacccugcaagaacacuacagcucccugaagggucugacauuguccuggaucggcgacggcaacaacauucuccauuccaucaugauguccgccgcaaaauucggcaugcaucuucaagccgccacgccgaaggguuacgagcccgacgcuuccgugacuaagcucgccgagcaguacgcuaaggagaacggaaccaagcuucugcugacuaacgacccacuagaagcagcccacgggggcaacgugcuuauuacugacaccuggaucuccaugggccaggaagaagagaaaaagaagcggcugcaggcguuccagggauaucaggucaccaugaaaaccgccaaggucgcugccuccgacuggaccuuccugcacugccugccucgcaagccugaagaaguggacgacgagguguucuacucgccacggagccucguguuccccgaggccgagaauagaaaguggaccaucauggccgugauggugucacugcucaccgacuacagcccgcagcuucagaagcccaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 105 - RNA, 1485 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugggccuugucaaucuccgcauccuccuuaacaacgccgcguuuagaaacggccacaacuucaugguccggaacuucagauguggccagccgcuucaaaaccggguccagcugaagggccgggaucuucugacccugaagaacuuuacuggcgaagagaucaaguacaugcucuggcucuccgcggacuugaaguuccgcauuaagcagaagggggaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucuggacacucuggccaaggaggcgucaauuccuauuaucaacggccuuagugaccucuaccauccgauucagauccuggccgauuaccucacccugcaagaacacuacagcucccugaagggucugacauuguccuggaucggcgacggcaacaacauucuccauuccaucaugauguccgccgcaaaauucggcaugcaucuucaagccgccacgccgaaggguuacgagcccgacgcuuccgugacuaagcucgccgagcaguacgcuaaggagaacggaaccaagcuucugcugacuaacgacccacuagaagcagcccacgggggcaacgugcuuauuacugacaccuggaucuccaugggccaggaagaagagaaaaagaagcggcugcaggcguuccagggauaucaggucaccaugaaaaccgccaaggucgcugccuccgacuggaccuuccugcacugccugccucgcaagccugaagaaguggacgacgagguguucuacucgccacggagccucguguuccccgaggccgagaauagaaaguggaccaucauggccgugauggugucacugcucaccgacuacagcccgcagcuucagaagcccaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 106 - RNA, 1374 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugggcggacuugucaaucuccgcauccuccuuaacaacgccgcguuuagaaacggccacaacuucaugguccggaacuucagauguggccagccgcuucaaaacaagguccagcugaagggccgggaucuucugacccugaagaacuuuacuggcgaagagaucaaguacaugcucuggcucuccgcggacuugaaguuccgcauuaagcagaagggggaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucuggacacucuggccaaggaggcgucaauuccuauuaucaacggccuuagugaccucuaccauccgauucagauccuggccgauuaccucacccugcaagaacacuacagcucccugaagggucugacauuguccuggaucggcgacggcaacaacauucuccauuccaucaugauguccgccgcaaaauucggcaugcaucuucaagccgccacgccgaaggguuacgagcccgacgcuuccgugacuaagcucgccgagcaguacgcuaaggagaacggaaccaagcuucugcugacuaacgacccacuagaagcagcccacgggggcaacgugcuuauuacugacaccuggaucuccaugggccaggaagaagagaaaaagaagcggcugcaggcguuccagggauaucaggucaccaugaaaaccgccaaggucgcugccuccgacuggaccuuccugcacugccugccucgcaagccugaagaaguggacgacgagguguucuacucgccacggagccucguguuccccgaggccgagaauagaaaguggaccaucauggccgugauggugucacugcucaccgacuacagcccgcagcuucagaagcccaaguucuagcucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 107 - RNA, 1377 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccauggcccuuuucaaucuccgcauccuccuuaacaacgccgcguuuagaaacggccacaacuucaugguccggaacuucagauguggccagccgcuucaaggcaagguccagcugaagggccgggaucuucugacccugaagaacuuuacuggcgaagagaucaaguacaugcucuggcucuccgcggacuugaaguuccgcauuaagcagaagggggaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucuggacacucuggccaaggaggcgucaauuccuauuaucaacggccuuagugaccucuaccauccgauucagauccuggccgauuaccucacccugcaagaacacuacagcucccugaagggucugacauuguccuggaucggcgacggcaacaacauucuccauuccaucaugauguccgccgcaaaauucggcaugcaucuucaagccgccacgccgaaggguuacgagcccgacgcuuccgugacuaagcucgccgagcaguacgcuaaggagaacggaaccaagcuucugcugacuaacgacccacuagaagcagcccacgggggcaacgugcuuauuacugacaccuggaucuccaugggccaggaagaagagaaaaagaagcggcugcaggcguuccagggauaucaggucaccaugaaaaccgccaaggucgcugccuccgacuggaccuuccugcacugccugccucgcaagccugaagaaguggacgacgagguguucuacucgccacggagccucguguuccccgaggccgagaauagaaaguggaccaucauggccgugauggugucacugcucaccgacuacagcccgcagcuucagaagcccaaguucuagauaagugaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 108 - RNA, 1377 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccauggcccuuuucaaucuccgcauccuccuuaacaacgccgcguuuagaaacggccacaacuucaugguccggaacuucagauguggccagccgcuucaaggccggguccagcugaagggccgggaucuucugacccugaagaacuuuacuggcgaagagaucaaguacaugcucuggcucuccgcggacuugaaguuccgcauuaagcagaagggggaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucuggacacucuggccaaggaggcgucaauuccuauuaucaacggccuuagugaccucuaccauccgauucagauccuggccgauuaccucacccugcaagaacacuacagcucccugaagggucugacauuguccuggaucggcgacggcaacaacauucuccauuccaucaugauguccgccgcaaaauucggcaugcaucuucaagccgccacgccgaaggguuacgagcccgacgcuuccgugacuaagcucgccgagcaguacgcuaaggagaacggaaccaagcuucugcugacuaacgacccacuagaagcagcccacgggggcaacgugcuuauuacugacaccuggaucuccaugggccaggaagaagagaaaaagaagcggcugcaggcguuccagggauaucaggucaccaugaaaaccgccaaggucgcugccuccgacuggaccuuccugcacugccugccucgcaagccugaagaaguggacgacgagguguucuacucgccacggagccucguguuccccgaggccgagaauagaaaguggaccaucauggccgugauggugucacugcucaccgacuacagcccgcagcuucagaagcccaaguucuagauaagugaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 109 - RNA, 1377 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccauggcccuuuucaaucuccgcauccuccuuaacaacgccgcguuuagaaacggccacaacuucaugguccggaacuucagauguggccagccgcuucaaggccggguccagcugaagggccgggaucuucugacccugaagaacuuuacuggcgaagagaucagguacaugcucuggcucuccgcggacuugaaguuccgcauuaagcagaagggggaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucuggacacucuggccaaggaggcgucaauuccuauuaucaacggccuuagugaccucuaccauccgauucagauccuggccgauuaccucacccugcaagaacacuacagcucccugaagggucugacauuguccuggaucggcgacggcaacaacauucuccauuccaucaugauguccgccgcaaaauucggcaugcaucuucaagccgccacgccgaaggguuacgagcccgacgcuuccgugacuaagcucgccgagcaguacgcuaaggagaacggaaccaagcuucugcugacuaacgacccacuagaagcagcccacgggggcaacgugcuuauuacugacaccuggaucuccaugggccaggaagaagagaaaaagaagcggcugcaggcguuccagggauaucaggucaccaugaaaaccgccaaggucgcugccuccgacuggaccuuccugcacugccugccucgcaagccugaagaaguggacgacgagguguucuacucgccacggagccucguguuccccgaggccgagaauagaaaguggaccaucauggccgugauggugucacugcucaccgacuacagcccgcagcuucagaagcccaaguucuagauaagugaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 110 - RNA, 1377 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccauggcccuugucaaucuccgcauccuccuuaacaacgccgcguuuagaaacggccacaacuucaugguccggaacuucagauguggccagccgcuucaaggcaggguccagcugaagggccgggaucuucugacccugaagaacuuuacuggcgaagagaucaaguacaugcucuggcucuccgcggacuugaaguuccgcauuaagcagaagggggaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucuggacacucuggccaaggaggcgucaauuccuauuaucaacggccuuagugaccucuaccauccgauucagauccuggccgauuaccucacccugcaagaacacuacagcucccugaagggucugacauuguccuggaucggcgacggcaacaacauucuccauuccaucaugauguccgccgcaaaauucggcaugcaucuucaagccgccacgccgaaggguuacgagcccgacgcuuccgugacuaagcucgccgagcaguacgcuaaggagaacggaaccaagcuucugcugacuaacgacccacuagaagcagcccacgggggcaacgugcuuauuacugacaccuggaucuccaugggccaggaagaagagaaaaagaagcggcugcaggcguuccagggauaucaggucaccaugaaaaccgccaaggucgcugccuccgacuggaccuuccugcacugccugccucgcaagccugaagaaguggacgacgagguguucuacucgccacggagccucguguuccccgaggccgagaauagaaaguggaccaucauggccgugauggugucacugcucaccgacuacagcccgcagcuucagaagcccaaguucuaagugaauagacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 111 - RNA, 1496 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccauggcccuuuucaaucuccgcauccuccuuaacaacgccgcguuuagaaacggccacaacuucaugguccggaacuucagauguggccagccgcuucaagucaagguccagcugaagggccgggaucuucugacccugaagaacuuuacuggcgaagagaucaaguacaugcucuggcucuccgcggacuugaaguuccgcauuaagcagaagggggaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucuggacacucuggccaaggaggcgucaauuccuauuaucaacggccuuagugaccucuaccauccgauucagauccuggccgauuaccucacccugcaagaacacuacagcucccugaagggucugacauuguccuggaucggcgacggcaacaacauucuccauuccaucaugauguccgccgcaaaauucggcaugcaucuucaagccgccacgccgaaggguuacgagcccgacgcuuccgugacuaagcucgccgagcaguacgcuaaggagaacggaaccaagcuucugcugacuaacgacccacuagaagcagcccacgggggcaacgugcuuauuacugacaccuggaucuccaugggccaggaagaagagaaaaagaagcggcugcaggcguuccagggauaucaggucaccaugaaaaccgccaaggucgcugccuccgacuggaccuuccugcacugccugccucgcaagccugaagaaguggacgacgagguguucuacucgccacggagccucguguuccccgaggccgagaauagaaaguggaccaucauggccgugauggugucacugcucaccgacuacagcccgcagcuucagaagcccaaguucuagauaagugaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 112 - RNA, 1377 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccauggcccuuuucaaucuccgcauccuccuuaacaacgccgcguuuagaaacggccacaacuucaugguccggaacuucagauguggccagccgcuucaagucaggguccagcugaagggccgggaucuucugacccugaagaacuuuacuggcgaagagaucaaguacaugcucuggcucuccgcggacuugaaguuccgcauuaagcagaagggggaauaccuuccgcugcuucaaggaaagagccucggcaugaucuuugagaagcgcucaaccaggacccgccuuucuacugaaacuggguucgcgcugcucgguggccaccccugcuuccugacgacccaggacauccaccucggagugaacgaaucccucaccgauaccgcccggguguuaucgagcauggcagaugccgugcuggccaggguguacaaacaguccgaucuggacacucuggccaaggaggcgucaauuccuauuaucaacggccuuagugaccucuaccauccgauucagauccuggccgauuaccucacccugcaagaacacuacagcucccugaagggucugacauuguccuggaucggcgacggcaacaacauucuccauuccaucaugauguccgccgcaaaauucggcaugcaucuucaagccgccacgccgaaggguuacgagcccgacgcuuccgugacuaagcucgccgagcaguacgcuaaggagaacggaaccaagcuucugcugacuaacgacccacuagaagcagcccacgggggcaacgugcuuauuacugacaccuggaucuccaugggccaggaagaagagaaaaagaagcggcugcaggcguuccagggauaucaggucaccaugaaaaccgccaaggucgcugccuccgacuggaccuuccugcacugccugccucgcaagccugaagaaguggacgacgagguguucuacucgccacggagccucguguuccccgaggccgagaauagaaaguggaccaucauggccgugauggugucacugcucaccgacuacagcccgcagcuucagaagcccaaguucuagauaagugaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 113 - RNA, 1282 nt, Artificial Sequence
  Synthetic construct
ggcagaaaaauuugcuacauuguuucacaaacuucaaauauuauucauuuauuuagaucuauuauuacaucaaaacaaaaagccgccaccaugcuguucaaccugcgcauccugcugaacaacgccgccuuccgcaacggccacaacuucauggugcgcaacuuccgcugcggccagccccugcagaacaaggugcagcugaagggccgcgaccugcugacccugaagaacuucaccggcgaggagaucaaguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggcuucgcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugaggucucuaguaaugagcuggagccucgguagccguuccuccugcccgcugggccucccaacgggcccuccuccccuccuugcaccggcccuuccuggucuuugaauaaagucugagugggcaucuag

SEQ ID NO: 114 - RNA, 1285 nt, Artificial Sequence
  Synthetic construct
ggcagaaaaauuugcuacauuguuucacaaacuucaaauauuauucauuuauuuagaucuauuauuacaucaaaacaaaaagccgccaccaugggaguauucaaccugcgcauccugcugaacaacgccgccuuccgcaacggccacaacuucauggugcgcaacuuccgcugcggccagccccugcagaacaaggugcagcugaagggccgcgaccugcugacccugaagaacuucaccggcgaggagaucaaguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggcuucgcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugaggucucuaguaaugagcuggagccucgguagccguuccuccugcccgcugggccucccaacgggcccuccuccccuccuugcaccggcccuuccuggucuuugaauaaagucugagugggcaucuag

SEQ ID NO: 115 - RNA, 1285 nt, Artificial Sequence
  Synthetic construct
ggcagaaaaauuugcuacauuguuucacaaacuucaaauauuauucauuuauuuagaucuauuauuacaucaaaacaaaaagccgccaccaugggaguauucaaccugcgcauccugcugaacaacgccgccuuccgcaacggccacaacuucauggugcgcaacuuccgcugcggccagccccugcagaaccgggugcagcugaagggccgcgaccugcugacccugaagaacuucaccggcgaggagauccgguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggcuucgcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugaggucucuaguaaugagcuggagccucgguagccguuccuccugcccgcugggccucccaacgggcccuccuccccuccuugcaccggcccuuccuggucuuugaauaaagucugagugggcaucuag

SEQ ID NO: 116 - RNA, 1285 nt, Artificial Sequence
  Synthetic construct
ggcagaaaaauuugcuacauuguuucacaaacuucaaauauuauucauuuauuuagaucuauuauuacaucaaaacaaaaagccgccaccaugcugguauucaaccugcgcauccugcugaacaacgccgccuuccgcaacggccacaacuucauggugcgcaacuuccgcugcggccagccccugcagaaccgggugcagcugaagggccgcgaccugcugacccugaagaacuucaccggcgaggagauccgguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggcuucgcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugaggucucuaguaaugagcuggagccucgguagccguuccuccugcccgcugggccucccaacgggcccuccuccccuccuugcaccggcccuuccuggucuuugaauaaagucugagugggcaucuag

SEQ ID NO: 117 - RNA, 1225 nt, Artificial Sequence
  Synthetic construct
auuauuacaucaaaacaaaaagccgccaccaugggaguauucaaccugcgcauccugcugaacaacgccgccuuccgcaacggccacaacuucauggugcgcaacuuccgcugcggccagccccugcagaacaaggugcagcugaagggccgcgaccugcugacccugaagaacuucaccggcgaggagaucaaguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggcuucgcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugaggucucuaguaaugagcuggagccucgguagccguuccuccugcccgcugggccucccaacgggcccuccuccccuccuugcaccggcccuuccuggucuuugaauaaagucugagugggcaucuag

SEQ ID NO: 118 - RNA, 1225 nt, Artificial Sequence
  Synthetic construct
auuauuacaucaaaacaaaaagccgccaccaugggaguauucaaccugcgcauccugcugaacaacgccgccuuccgcaacggccacaacuucauggugcgcaacuuccgcugcggccagccccugcagaaccgggugcagcugaagggccgcgaccugcugacccugaagaacuucaccggcgaggagauccgguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggcuucgcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugaggucucuaguaaugagcuggagccucgguagccguuccuccugcccgcugggccucccaacgggcccuccuccccuccuugcaccggcccuuccuggucuuugaauaaagucugagugggcaucuag

SEQ ID NO: 119 - RNA, 1225 nt, Artificial Sequence
  Synthetic construct
auuauuacaucaaaacaaaaagccgccaccaugcugguauucaaccugcgcauccugcugaacaacgccgccuuccgcaacggccacaacuucauggugcgcaacuuccgcugcggccagccccugcagaaccgggugcagcugaagggccgcgaccugcugacccugaagaacuucaccggcgaggagauccgguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggcuucgcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugaggucucuaguaaugagcuggagccucgguagccguuccuccugcccgcugggccucccaacgggcccuccuccccuccuugcaccggcccuuccuggucuuugaauaaagucugagugggcaucuag

SEQ ID NO: 120 - RNA, 1376 nt, Artificial Sequence
  Synthetic construct
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccauguuguucaacuugaggaucuuguugaacaacgccgccuucaggaacggacacaacuucaugguaaggaacuucaggugcggacagcccuugcagaacaaaguacaguugaaaggaagggacuuguugacauugaaaaacuucacaggagaagaaaucaaauacauguugugguugucggccgacuugaaauucaggaucaaacagaaaggagaauacuugcccuuguugcagggaaaaucguugggaaugaucuucgaaaaaaggucgacaaggacaagguugucgacagaaacaggauucgccuuguugggaggacaccccugcuucuugacaacacaggacauccacuugggaguaaacgaaucguugacagacacagccaggguauugucgucgauggccgacgccguauuggccaggguauacaaacagucggacuuggacacauuggccaaagaagccucgauccccaucaucaacggauugucggacuuguaccaccccauccagaucuuggccgacuacuugacauugcaggaacacuacucgucguugaaaggauugacauugucguggaucggagacggaaacaacaucuugcacucgaucaugaugucggccgccaaauucggaaugcacuugcaggccgccacacccaaaggauacgaacccgacgccucgguaacaaaauuggccgaacaguacgccaaagaaaacggaacaaaauuguuguugacaaacgaccccuuggaagccgcccacggaggaaacguauugaucacagacacauggaucucgaugggacaggaagaagaaaaaaaaaaaagguugcaggccuuccagggauaccagguaacaaugaaaacagccaaaguagccgccucggacuggacauucuugcacugcuugcccaggaaacccgaagaaguagacgacgaaguauucuacucgcccaggucguugguauuccccgaagccgaaaacaggaaauggacaaucauggccguaaugguaucguuguugacagacuacucgccccaguugcagaaacccaaauucugaauagugaacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuag

SEQ ID NO: 121 - RNA, 1222 nt, Artificial Sequence
  Synthetic construct
auuauuacaucaaaacaaaaagccgccaccaugcuguucaaccugcgcauccugcugaacaacgccgccuuccgcaacggccacaacuucauggugcgcaacuuccgcugcggccagccccugcagggcaaggugcagcugaagggccgcgaccugcugacccugaagaacuucaccggcgaggagaucaaguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggcuucgcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugaggucucuaguaaugagcuggagccucgguagccguuccuccugcccgcugggccucccaacgggcccuccuccccuccuugcaccggcccuuccuggucuuugaauaaagucugagugggcaucuag

SEQ ID NO: 122 - RNA, 1222 nt, Artificial Sequence
  Synthetic construct
auuauuacaucaaaacaaaaagccgccaccaugcuguucaaccugcgcauccugcugaacaacgccgccuuccgcaacggccacaacuucauggugcgcaacuuccgcugcggccagccccugcagggccgggugcagcugaagggccgcgaccugcugacccugaagaacuucaccggcgaggagaucaaguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggcuucgcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugaggucucuaguaaugagcuggagccucgguagccguuccuccugcccgcugggccucccaacgggcccuccuccccuccuugcaccggcccuuccuggucuuugaauaaagucugagugggcaucuag

SEQ ID NO: 123 - RNA, 1222 nt, Artificial Sequence
  Synthetic construct
auuauuacaucaaaacaaaaagccgccaccaugcuguucaaccugcgcauccugcugaacaacgccgccuuccgcaacggccacaacuucauggugcgcaacuuccgcugcggccagccccugcagggccgggugcagcugaagggccgcgaccugcugacccugaagaacuucaccggcgaggagauccgguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggcuucgcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugaggucucuaguaaugagcuggagccucgguagccguuccuccugcccgcugggccucccaacgggcccuccuccccuccuugcaccggcccuuccuggucuuugaauaaagucugagugggcaucuag

SEQ ID NO: 124 - RNA, 1282 nt, Artificial Sequence
  Synthetic construct
ggcagaaaaauuugcuacauuguuucacaaacuucaaauauuauucauuuauuuagaucuauuauuacaucaaaacaaaaagccgccaccaugcuguucaaccugcgcauccugcugaacaacgccgccuuccgcaacggccacaacuucauggugcgcaacuuccgcugcggccagccccugcagggcaaggugcagcugaagggccgcgaccugcugacccugaagaacuucaccggcgaggagaucaaguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggcuucgcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugaggucucuaguaaugagcuggagccucgguagccguuccuccugcccgcugggccucccaacgggcccuccuccccuccuugcaccggcccuuccuggucuuugaauaaagucugagugggcaucuag

SEQ ID NO: 125 - RNA, 21 nt, Artificial Sequence
  Synthetic construct
cacaaagaguaaagaagaaca

SEQ ID NO: 126 - RNA, 21 nt, Artificial Sequence
  Synthetic construct
aacacuaaaaguagaagaaaa

SEQ ID NO: 127 - RNA, 21 nt, Artificial Sequence
  Synthetic construct
cucagaaagauaagaucagcc

SEQ ID NO: 128 - DNA, 1065 bp, Artificial Sequence; identical to SEQ ID NO: 2
  Synthetic construct

SEQ ID NO: 129 - DNA, 1065 bp, Artificial Sequence
  Synthetic construct
atgctctttaatctgcgcatcttactgaacaacgccgcattccggaacggtcacaacttcatggtccgcaatttccgctgtggccagccgcttcaaaacaaggtccagctgaagggacgggatctgctgacactgaagaacttcaccggagaagagatcaagtacatgctgtggctcagcgcagacttgaagttccggatcaagcagaagggagaatacttgcccctgctgcaaggaaagtcgctgggaatgatttttgagaagcggtcaactcgcaccagactctccaccgaaactggtttcgcactgcttggcgggcacccttgcttcctgacgactcaggacatccacctcggcgtgaacgaatcgctaaccgataccgccagagtgctttcttccatggccgacgcggtgctggccagggtgtacaagcagtccgacctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcgtccgtgaccaagttggcggaacagtacgcgaaggagaacggaaccaagcttctgctgactaacgaccccctcgaggctgcgcatgggggcaacgtgctgattaccgacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctag

SEQ ID NO: 130 - DNA, 1065 bp, Artificial Sequence
  Synthetic construct
atgctgtttaacctacgtattttgctcaacaatgcagcctttagaaacggacataactttatggttcgaaactttcgctgcgggcagccactgcagaacaaggtccagctgaaagggagagatttgctcacgctgaagaactttactggcgaagaaatcaagtatatgctgtggttgtccgcggacctcaagtttcggattaagcagaaaggggagtatctgccactgctgcaaggaaagagcctcggcatgatcttcgagaagcggagcactcggaccaggctgagtaccgaaactggcttcgcattgttgggtggacatccatgttttctgacaacgcaggacattcatctgggcgtgaacgagagtctgacggacacagctcgcgttctgtcctctatggctgatgcggtgttggcccgggtctataagcagtccgatttggacaccttggctaaggaagctagcataccgattatcaatgggctgtccgacctgtatcaccctattcaaatcctggccgactacctcacactgcaagaacactatagctcattgaagggactgaccctgagctggataggggacggaaacaacatcctacatagcattatgatgtccgctgccaagtttggcatgcatcttcaagccgccacgccaaagggttatgagcccgacgcgtcagtgacaaagctggccgagcagtacgctaaggagaatggtaccaaattactgctgactaatgatccactggaggctgcacatggcggcaatgtactgatcaccgacacatggatctcgatgggccaggaggaagaaaagaagaagaggcttcaggccttccaaggctaccaggtcaccatgaaaacagctaaggttgcagcatctgattggacctttctgcactgtctgccaaggaagcccgaagaggtggacgatgaagtattctatagcccacggagtttggtgttccctgaggctgaaaataggaagtggacaattatggccgtaatggtgtccctgttaaccgactactctccgcaactgcagaaacctaagttttag

SEQ ID NO: 131 - DNA, 1065 bp, Artificial Sequence
  Synthetic construct
atgctgtttaacttaaggatcctgctgaacaacgccgcttttcgtaacggtcataactttatggtccggaactttagatgtggccagccgctgcagaacaaggttcagctgaaggggagggatctgctgaccttgaagaactttaccggcgaagagatcaagtacatgttgtggctgagcgccgatctgaagtttaggattaagcagaagggggagtatttgccactgctgcaaggaaaatccttggggatgatcttcgagaagcgctccactagaacccggctaagcacagaaaccggcttcgcacttctgggtggacatccctgttttctgacgacgcaggatatacacctgggcgtgaatgagagtctgacggacacagctagggtgttgagcagcatggccgatgcagtactggcccgcgtttataagcagagcgacttggacacactggccaaggaagcgtcaattccgattatcaatgggctgtcagacctgtatcatcccattcaaatcttggctgactatctgaccctgcaagaacattacagctccctgaagggcctcacgttgtcctggattggcgacggaaacaacattctgcattcgatcatgatgagcgctgctaagtttggcatgcacctccaagccgctacacctaagggatatgagcctgatgccagcgtaaccaagctggccgaacagtacgcgaaggagaatggcacgaaactgctgttgacaaatgacccactggaggcagctcacggtggcaacgtgctgatcaccgacacgtggatatctatgggacaggaagaagagaagaagaagcggctgcaggcattccaagggtatcaggtcaccatgaaaacggccaaggttgctgcatccgactggacatttctgcattgcttgccccgcaaaccagaagaagtagacgacgaagtcttttattccccacggtcgctggtgttccccgaggcggagaatcgaaagtggacgattatggccgtgatggtgtccctgctgactgattactctccccaactgcaaaagcctaagttttag

SEQ ID NO: 132 - DNA, 1065 bp, Artificial Sequence
  Synthetic construct
atgcttttcaacctgaggatcctcctgaacaacgccgcctttcgcaatggtcacaactttatggtccggaacttcagatgcggccagccgctgcagaacaaggtccagctgaagggacgggatctgctgactctgaagaacttcaccggagaagagatcaagtacatgctgtggctgtcggccgacctgaagttcaggatcaagcagaagggagaatacctcccgctgctgcaaggaaagtccctgggcatgattttcgagaagcgctcgaccagaactcggttgtccaccgaaaccgggtttgcgctgctgggcggacatccttgcttcctgacgactcaggatattcacctgggagtgaacgagtcgctgaccgacaccgccagagtgctgagctcgatggccgacgccgtgttggcacgcgtgtacaagcagtccgatctggataccctggccaaagaagcttccatcccgatcattaacgggctgagcgacctctaccaccccattcaaatcctggccgactacctgactctgcaagaacactacagctcgctgaaggggttgactctgtcctggatcggcgacggaaacaacatcctgcactccatcatgatgtcggccgcaaagttcggcatgcatttgcaagccgccaccccaaagggctacgaaccagacgcgagcgtcaccaagctggccgaacagtacgcgaaggaaaatggtactaagctgctgctgaccaacgacccattggaagctgcccatggtggaaacgtgctgatcaccgacacctggatctcgatgggccaggaagaggagaagaagaagcggctgcaggcgttccaggggtatcaggtcaccatgaaaacagccaaagtggcagcgtcagactggaccttcctccactgtctgcctcgcaagccagaggaggtggacgacgaggtgttctactcccctcggtccctcgtgttccctgaggctgagaaccggaagtggaccattatggccgtgatggtgtcactcctgactgattactccccgcaactgcagaagcccaagttctag

SEQ ID NO: 133 - DNA, 1065 bp, Artificial Sequence
  Synthetic construct
atgctgtttaacctgaggatcctattgaacaatgctgcttttcgtaatggccataactttatggttcggaactttagatgcgggcagccactgcagaacaaggtccagttgaaaggccgcgatctgttgacattgaagaactttaccggcgaagagattaagtatatgctgtggctgtctgctgacctcaagtttcgaatcaagcagaagggcgaatatctccccctgctgcaaggaaagtctctcggcatgatctttgagaagcggagtacccgaacacggctgagcaccgaaacgggcttcgcactgctggggggccatccctgttttctgacaacgcaggacatccacttgggggttaacgaatcattgactgataccgcccgcgtactgtcatccatggccgacgctgtgctggctagggtgtacaagcagtcagatctggatacactggccaaggaagctagcataccaatcatcaatggactgagtgacctttatcacccgattcaaatactagccgattatctgaccctgcaagagcattactcctcgctgaaaggcctcacgctgtcctggatcggcgacggcaacaacattctgcatagtattatgatgtctgctgccaaattcggcatgcatctgcaagctgctacgccgaagggttatgaacccgacgcgtcagttacgaagctcgctgagcagtatgcaaaggagaatggcacaaagctgttgcttaccaacgatcccctggaagctgctcatggcggcaatgtgctgattactgacacctggatttcaatgggccaggaggaggagaagaagaagaggttacaggcttttcaaggttaccaagtcacgatgaaaaccgctaaggtcgcagccagcgactggacattcctgcactgtctgccaagaaagccggaagaagtggacgacgaggtgttctattccccgcggtctttggtgtttccggaggccgaaaacaggaaatggaccattatggccgtgatggtatcgttgctgacggactacagccctcagttgcaaaagcccaagttctag

SEQ ID NO: 134 - DNA, 1065 bp, Artificial Sequence
  Synthetic construct
atgctctttaacctccgcatcctcctcaacaacgccgccttccggaatgggcataacttcatggtccggaacttcagatgcggccagcccctgcaaaacaaggtccagttgaagggacgggacctccttacgctgaagaactttaccggagaagagattaagtacatgctgtggttgtccgctgacctcaagttccgcattaagcagaagggagaatatctgccgctgctgcaaggaaagagcctgggcatgatcttcgaaaagcgctccactagaacccggctgtcgactgagactggattcgccttgctcggtggacacccgtgcttcctgacgacccaggacatccacctgggagtgaacgagtcacttacggataccgcgagggtgctgtcctcaatggccgacgcagtgctcgcgcgcgtgtacaagcagtcagatctggataccctggccaaggaagccagcattcccatcatcaacggactgagcgacctttaccacccaatccagatcctcgccgactacttaaccctgcaagagcactacagctccctgaagggactgactctgtcctggatcggggatggaaacaacatcctgcactccatcatgatgtctgccgctaagtttgggatgcatctgcaagccgcaacccctaagggatacgagcccgacgcctcggtgaccaaacttgcggaacagtacgccaaggaaaacggtaccaagctgctgctgaccaacgaccctctggaagcggcccacggaggaaatgtgctgattaccgacacctggatttcgatgggccaggaggaggagaagaagaagagactgcaggcgttccagggatatcaggtcaccatgaaaaccgccaaggtcgccgccagcgactggaccttcctgcactgtctccctcggaaaccggaagaagtggatgacgaggtgttctactccccgcgctcgctggtgttcccggaggctgaaaacaggaagtggacaatcatggccgtgatggtgtccctgttgaccgactactccccacaactgcagaagcccaagttctag

SEQ ID NO: 135 - DNA, 1065 bp, Artificial Sequence
  Synthetic construct
atgcttttcaatctgcgcatcctcctgaacaacgccgccttccgcaatggacacaactttatggtccgcaacttccgctgtgggcagccgctgcagaacaaggtccagctcaaggggagagatctcctgaccctgaagaacttcactggagaggagatcaagtacatgctgtggctgtccgccgacctgaaatttcggattaagcagaagggcgaatacctcccactgctgcaaggaaagtctttgggcatgatcttcgaaaagagaagcacccggacccggttgagcaccgaaactgggttcgcgctcctcggtggacacccgtgcttcctgaccacccaagatattcatctgggtgtcaacgaaagcctgaccgacaccgccagggtgctgtcatccatggctgacgcagtgctcgcccgggtgtacaagcagtcagacctggacaccctcgccaaggaagcttcgatccctatcatcaacggactttccgacctgtaccaccccatccaaattctggccgactacctgactctgcaagaacactatagctcgctgaaaggacttactctgtcctggatcggggacggcaacaacattctccattccatcatgatgtccgctgccaagttcggaatgcaccttcaagcagcgactcccaagggatacgaacctgatgcctccgtgactaagctggcagagcagtacgccaaggagaacggtacaaagctgctgctcacgaacgaccccctggaggcggcccacggcggaaacgtgctgattaccgatacctggatctcaatgggccaggaagaggagaagaagaagcggctccaggcgtttcaaggctaccaggtcaccatgaaaaccgcgaaggtcgccgcctccgactggactttcttgcactgcctgccgcggaagcccgaggaagtggatgacgaagtgttctactcgccgagatcgttggtgttccctgaggccgaaaacaggaagtggaccatcatggccgtgatggtgtccctgctgactgattacagcccacagctgcagaagcctaagttctag

SEQ ID NO: 136 - DNA, 1065 bp, Artificial Sequence
  Synthetic construct
atgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctag

SEQ ID NO: 137 - DNA, 1065 bp, Artificial Sequence
  Synthetic construct
atgcttttcaacctgagaatcctcctgaacaacgccgccttccgcaatggtcataacttcatggtccgcaactttcgctgcggacagcctctccaaaacaaggtccagctcaaggggcgcgacctcctcacactgaagaacttcactggagaagaaatcaagtacatgctgtggctgagcgccgatctgaagttccggatcaagcagaagggagagtaccttcctctgctgcaagggaagtccttgggaatgattttcgagaagcggtccacccggaccaggctgagcactgaaactggcttcgccctgctgggaggccacccttgtttcctgaccactcaggacatccacctgggcgtgaacgagtccctgaccgatactgccagagtgctgtcctccatggccgacgccgtgctcgcccgggtgtacaagcagtcagacctcgatacgctggccaaggaagcctccattcccattatcaatggtctgtcggacctctaccatccaatccaaatcctcgccgactacctgactctgcaagaacactacagctcactcaagggcctcaccctctcctggatcggcgacggaaacaacatccttcactcgattatgatgtcggccgcgaagttcgggatgcacctccaagctgccactccaaaaggctacgagccggatgcctcagtgactaagttggcggaacagtatgcgaaggagaacggtaccaagctcctgctgactaacgacccgctggaggccgcccacgggggaaacgtgctcatcaccgatacttggatttccatgggacaggaggaagagaagaagaagcggttgcaggcatttcagggctaccaggtcaccatgaaaactgccaaagtcgccgccagcgactggaccttcctgcactgcctgccccgcaagcctgaagaagtggacgacgaggtgttctactctccccggtccctcgtgttccctgaggccgaaaacaggaagtggaccatcatggctgtgatggtgtccctcctgaccgactacagccctcagctccaaaaacccaagttttag

SEQ ID NO: 138 - DNA, 1065 bp, Artificial Sequence
  Synthetic construct
atgcttttcaacctgagaatcctcttgaacaatgctgcttttcggaatggccacaactttatggttcggaacttccgttgcggccagcctttacaaaacaaggtccagctgaagggccgggatttgctcacactgaagaactttactggggaggagattaagtatatgctgtggctgtccgctgacctgaagtttaggatcaagcagaagggcgaatatctgccgctgctgcaagggaaaagtctgggcatgatttttgaaaagcgctctacccggaccagactgtctacggaaacaggctttgccctgctgggcggccacccctgttttctgacaacgcaggacatccatctgggcgtgaacgaatcactgaccgatactgctcgggtactcagttctatggctgacgcagtgctggctagggtgtacaagcagagcgacttggacacactggctaaggaggccagcatccccattatcaatggcctgtctgatttgtaccatcccattcaaatcctggctgattatctgacactacaagagcattactcaagtctgaagggtttgactctctcctggatcggcgacggcaacaacattttacattccattatgatgagtgctgctaagtttggcatgcatttgcaagctgctaccccaaagggctatgaacctgacgctagcgtaaccaagttggccgaacagtatgctaaagagaatggcaccaagctgctcctgacgaatgaccccctggaagctgctcatggcggaaacgtacttataactgatacatggattagcatgggccaggaagaggagaagaagaagagactgcaggccttccaaggctatcaggtcaccatgaaaactgccaaggttgcagctagcgactggaccttcctgcactgtttgccgaggaaacccgaggaggtggacgatgaagtcttttattctccccgctccttggtgtttcccgaggctgaaaatcgaaagtggacgataatggcagtgatggtgtccctactgaccgactattctccacaactgcagaagcctaaattctag

SEQ ID NO: 139 - DNA, 1065 bp, Artificial Sequence
  Synthetic construct
atgcttttcaatctgaggatcctgctgaacaacgctgcttttcgcaacggtcataactttatggttcgcaattttcgttgtggccagccgctgcagaacaaggttcagctgaagggcagagatctgctgactctgaagaacttcactggggaagaaatcaagtatatgttatggctgtccgcggatctgaaatttcgaatcaagcagaagggcgaatatcttcccctgctgcaagggaaatccttgggcatgatttttgagaagaggagcactaggactagattgtcaacagaaacaggctttgctttgttgggcggacatccctgctttctgacgacacaggatatccacctcggcgtaaacgagtccctcaccgacactgctagggtactgagcagcatggccgacgctgtgctagcccgggtttacaagcagtcagacctggacacccttgccaaggaagcttctattccaattatcaacggcctgagtgacctgtatcaccctattcaaatactcgccgactatttgacgcttcaagaacattacagcagcctcaagggcttaaccttgagttggataggcgacggcaacaatatcctgcattccattatgatgtctgccgctaagtttggcatgcatctacaagccgcaacacccaagggctatgaacccgacgctagcgtgaccaagctggccgagcagtatgctaaggaaaatggcacaaagctccttcttaccaacgatcccctggaggctgctcacggcggcaacgtgctgattaccgatacatggattagcatgggccaggaggaggagaaaaagaagcggctccaggcttttcaaggctatcaggtcaccatgaaaactgcaaaggtcgctgcctccgactggactttcctgcattgtctaccccgcaagcctgaggaagtggacgatgaggtgttctactccccacggagtctggtgttcccggaagcagagaatcggaagtggaccatcatggctgtcatggtgtcgctcttgactgactattctccccaactgcaaaaacccaagttttag

SEQ ID NO: 140 - DNA, 1065 bp, Artificial Sequence; identical to SEQ ID NO: 139
  Synthetic construct

SEQ ID NO: 141 - DNA, 1065 bp, Artificial Sequence
  Synthetic construct
atgctttttaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccacgccaaaaggatacgaaccggatgcgtccgtgacgaagttggcggaacagtacgcgaaggagaacggaaccaagcttctgctgactaacgaccccctcgaggctgcgcatgggggcaacgtgctgattaccgacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctag

SEQ ID NO: 142 - DNA, 1065 bp, Artificial Sequence
  Synthetic construct
atgcttttcaacctgagaatcctcttgaacaatgctgcttttcggaatggccacaactttatggttcggaacttccgttgcggccagcctttacaaaacaaggtccagctgaagggccgggatttgctcacactgaagaactttactggagaagagatcaagtacatgctgtggctgtcggccgacctgaagttcaggatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcgtccgtgaccaagttggcggaacagtacgcgaaggagaacggaaccaagcttctgctgactaacgaccccctcgaggctgcgcatgggggcaacgtgctgattaccgacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctag

SEQ ID NO: 143 - DNA, 1065 bp, Artificial Sequence
  Synthetic construct
atgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcatccgtgaccaagttggcggaacagtacgcgaaggagaacggaaccaagctcctgctgactaacgacccgctcgaggctgcgcatgggggtaacgtgctgattacggacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctag

SEQ ID NO: 144 - DNA, 1065 bp, Artificial Sequence
  Synthetic construct
atgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcgtccgtgaccaagttggcggaacagtacgcgaaggagaacggaaccaagcttctgctgactaacgaccccctcgaggctgcgcatgggggcaacgtgctgattaccgacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctag

SEQ ID NO: 145 - DNA, 1065 bp, Artificial Sequence
  Synthetic construct
atgctgttcaacctgcgaatcctgctgaacaacgccgcttttcggaacgggcacaactttatggtgaggaactttcgctgcggacagcccctccagaataaggtccagctgaagggcagggacctgctgaccctgaaaaatttcacaggggaggaaatcaagtatatgctgtggctgtcagctgatctgaagttccggatcaagcagaagggcgaatatctgcctctgctccagggcaaaagcctggggatgatcttcgaaaagcgcagtactcggaccagactgtcaaccgagactggattcgctctgctgggaggacacccttgttttctgaccactcaggacattcacctgggagtgaacgagtccctgaccgacactgctcgcgtcctgagctctatggccgacgctgtgctggctcgagtctacaaacagtccgacctggataccctggccaaggaagcttctatcccaattattaacggcctgtcagacctgtatcaccccatccagattctggccgattacctgaccctccaggagcactattctagtctgaaagggctgacactgagttggattggggacggaaacaatatcctgcactctattatgatgtcagccgccaagtttggaatgcacctccaggctgcaaccccaaaaggctacgaacccgatgcctcagtgacaaagctggctgaacagtacgccaaagagaacggcactaagctgctgctgaccaacgaccctctggaggccgctcacggaggcaacgtgctgatcaccgatacctggattagtatgggacaggaggaagagaagaagaagcggctccaggccttccagggctaccaggtgacaatgaaaaccgctaaggtcgcagccagcgattggacctttctgcactgcctgcccagaaagcccgaagaggtggacgacgaggtcttctactctcccagaagcctggtgtttcccgaagctgagaataggaagtggacaattatggcagtgatggtcagcctgctgactgattattcacctcagctccagaaaccaaagttctga

SEQ ID NO: 146 - DNA, 1065 bp, Artificial Sequence
  Synthetic construct
atgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 147 - DNA, 1065 bp, Artificial Sequence
  Synthetic construct
atgctgttcaacctgcgaatcctgctgaacaacgccgcttttcggaacgggcacaactttatggtgaggaactttcgctgcggacagcccctccagaataaggtccagctgaagggcagggacctgctgaccctgaaaaatttcacaggggaggaaatcaagtatatgctgtggctgtcagctgatctgaagttccggatcaagcagaagggcgaatatctgcctctgctccagggcaaaagcctggggatgatcttcgaaaagcgcagtactcggaccagactgtcaaccgagactggattcgctctgctgggaggacacccttgttttctgaccactcaggacattcacctgggagtgaacgagtccctgaccgacactgctcgcgtcctgagctctatggccgacgctgtgctagctcgagtctacaaacagtccgacctggataccctggccaaggaagcttctatcccaattattaacggcctgtcagacctgtatcaccccatccagattctggccgattacctgaccctccaggagcactattctagtctgaaagggctgacactgagttggattggggacggaaacaatatcctgcactctattatgatgtcagccgccaagtttggaatgcacctccaggctgcaaccccaaaaggctacgaacccgatgcctcagtgacaaagctggctgaacagtacgccaaagagaacggcactaagctgctgctgaccaacgaccctctggaggccgctcacggaggcaacgtgctgatcaccgatacctggattagtatgggacaggaggaagagaagaagaagcggctccaggccttccagggctaccaggtgacaatgaaaaccgctaaggtcgcagccagcgattggacctttctgcactgcctgcccagaaagcccgaagaggtggacgacgaggtcttctactctcccagaagcctggtgtttcccgaagctgagaataggaagtggacaattatggcagtgatggtcagcctgctgactgattattcacctcagctccagaaaccaaagttctga

SEQ ID NO: 148 - DNA, 1065 bp, Artificial Sequence
  Synthetic construct
atgctgttcaacctgcgaatcctgctgaacaatgccgcttttcggaacgggcacaatttcatggtgaggaactttcgctgcggacagcccctccagaacaaggtccagctgaagggcagggacctgctgaccctgaaaaatttcacaggggaggaaatcaagtacatgctgtggctgtcagccgatctgaagttccggatcaagcagaagggcgaatatctgcctctgctccagggcaaaagcctggggatgatcttcgaaaagcgcagtactcggaccagactgtcaacagagactggattcgcactgctgggaggacacccatgttttctgaccacacaggacattcatctgggagtgaacgagtccctgaccgacacagcacgcgtcctgagctccatggctgatgcagtgctggctcgagtctacaaacagtctgacctggataccctggccaaggaagcttctatcccaatcattaatggcctgagtgacctgtatcaccccatccagattctggccgattacctgaccctccaggagcattattctagtctgaaagggctgacactgagctggattggggacggaaacaatatcctgcactccattatgatgagcgccgccaagtttggaatgcacctccaggctgcaaccccaaaaggctacgaacccgatgcctccgtgacaaagctggcagaacagtatgccaaagagaacggcactaagctgctgctgaccaatgaccctctggaggccgctcacggaggcaacgtgctgatcactgatacctggattagtatgggacaggaggaagagaagaagaagcggctccaggccttccagggctaccaggtgacaatgaaaactgctaaggtcgcagccagcgactggacctttctgcattgcctgcccagaaagcctgaagaggtggacgatgaggtcttctactcacccagaagcctggtgtttcctgaagctgagaataggaagtggacaatcatggcagtgatggtcagcctgctgactgattattcccctcagctccagaaaccaaagttctga

SEQ ID NO: 149 - DNA, 1065 bp, Artificial Sequence
  Synthetic construct
atgcttttcaaccttcgcattctcctcaacaacgccgcgtttagaaacggacacaacttcatggtccgcaacttccgctgcggacagccgctgcagaacaaggtccagctcaagggtcgggatctcctgacgctgaagaactttaccggcgaagagattaagtacatgctgtggctgtccgccgaccttaagttccggatcaagcagaagggcgaataccttcccctgctgcaaggaaagtccctgggcatgatcttcgagaagcgcagtaccagaaccagactctccactgaaaccgggttcgcgctgcttggcggccacccgtgtttcctcactacgcaagacatccatcttggcgtgaacgagtcccttaccgacaccgccagggtgctgtcaagcatggccgacgccgtccttgcgcgcgtgtacaagcagtcagaccttgatactctggccaaggaagcctccatccctattatcaacggcctatccgacctttaccacccgatccagatcctcgctgactacctgaccctgcaagaacactacagcagcctcaagggactgactctgtcctggatcggcgacgggaacaacatcctgcactcaatcatgatgagcgcagccaagttcggcatgcatctccaagccgctacacccaagggttatgaaccggacgcctctgtgaccaagttggcagaacagtacgccaaggagaacggtactaagctccttttaaccaacgaccccctcgaagcagcccatggcgggaatgtgctcattaccgatacctggatttcgatgggccaggaggaggagaagaagaagcggctgcaggcgttccagggctaccaggtcaccatgaaaactgccaaagtggccgcctcggattggacctttctccactgcctgcctcggaagcctgaggaggtggacgacgaagtgttctactccccacggtccctcgtgttccccgaggccgaaaataggaagtggaccatcatggccgtgatggtgtccctcttgaccgattacagcccgcagcttcagaagcctaaattctag

SEQ ID NO: 150 - DNA, 1065 bp, Artificial Sequence
  Synthetic construct
atgcttttcaatcttcgcatcctgttgaacaacgccgccttccgcaatggtcacaacttcatggtccggaacttcagatgtggacagcctctccaaaacaaggtccagctgaagggaagggacctcttaaccctcaaaaactttactggagaggagatcaagtacatgctgtggcttagcgccgaccttaagttccggatcaagcagaagggagagtacctcccgctgctgcaaggaaagagtcttggaatgatcttcgagaagcggtccaccagaactcgcctctccactgaaaccggattcgcactcctgggtggacacccgtgctttctgaccacccaagacatccacctcggagtgaacgagagcctcacggacaccgcgagagtgctgtcatccatggccgacgccgtgcttgcacgggtctacaagcagtccgatctggacactcttgccaaggaagcctccattcctatcattaacggtctgtcggatctgtaccacccgattcagatccttgcggactacctcacacttcaagaacactattcaagcctaaagggtctgaccctgtcctggatcggagatggaaacaacattctccattccatcatgatgagcgctgccaagttcggaatgcatctccaagcagcgactcctaagggttacgagccggacgcctcagtgactaagctggccgagcagtacgccaaggagaacggtaccaaactgttgcttactaacgacccgcttgaagcggcccatggaggaaacgtgctgattaccgacacctggatttcgatgggacaggaagaggagaagaagaagcggctccaggcgttccagggataccaggtcaccatgaaaacggccaaagtggccgctagcgattggacctttctgcactgcctcccgcgcaagcctgaagaagtggacgacgaagtgttctactcccctcgctctcttgtgttcccggaagccgaaaacaggaagtggaccatcatggccgtgatggtgtccctcctgaccgattacagcccgcagctgcagaagcctaagttctag

SEQ ID NO: 151 - DNA, 1065 bp, Artificial Sequence
  Synthetic construct
atgcttttcaatctccgcatcctcctcaacaacgccgcgtttagaaacggccacaacttcatggtccggaacttcagatgtggccagccgcttcagaacaaggtccagctcaagggccgggatcttctgaccctgaagaactttactggcgaagaaatcaagtacatgctctggctctccgccgacttgaagttccgcattaagcagaagggggaataccttccgctgctgcaaggaaagtcgctcggcatgatctttgagaagcgctcaacccgcaccaggctgtccactgaaaccgggttcgcgctgcttggtggccacccctgcttcctgaccacccaagacattcacctcggagtgaacgaatcgctcactgatactgcccgggtgctgtcgtcgatggccgatgcagtgctggccagggtgtacaaacagtccgatctggacactctggccaaggaggcgtccatccctattatcaacggcctttccgacctctaccacccgattcagatccttgccgattacctcaccctgcaagaacactactcgtcactgaagggtctgaccttgtcctggatcggcgacggcaacaacatcctccattccattatgatgtccgccgccaaattcggcatgcatcttcaagccgcaacccctaagggttacgagccggacgcttccgtgaccaagctcgccgagcagtacgctaaggagaacggaaccaagcttctgctgactaacgaccccctagaggcagcccacgggggcaacgtgcttattactgacacctggatctccatgggacaggaagaagagaagaagaagcggttacaggcgttccagggctatcaggtcaccatgaaaaccgccaaggtcgctgcctcggactggaccttcctgcattgcctgcctcgcaagcccgaagaagtggacgacgaggtgttctactcgccacggtcccttgtgttccctgaggccgagaatagaaagtggaccattatggccgtgatggtgtcccttctcaccgactactcgccgcaactgcagaaacccaagttctag

SEQ ID NO: 152 - DNA, 1065 bp, Artificial Sequence
  Synthetic construct
atgcttttcaatcttcgcatcctcctcaacaacgccgccttccggaacggtcacaacttcatggtccggaacttccgctgcggccagccgctccaaaacaaagtgcagcttaagggccgcgatctcctgaccctgaagaacttcaccggagaggaaatcaagtacatgctgtggctctcggcggacctgaagtttaggattaagcagaagggggagtatctgccgctgctccaagggaagtcccttggcatgatcttcgaaaagaggtccacccggactcggctcagcaccgaaacaggttttgcacttctggggggccacccgtgcttcctgacgacccaggacatccatctgggtgtcaacgagagtttgaccgacactgccagagtgctgtcatccatggcggacgcggtgctcgcgagagtgtacaagcagtccgatcttgacaccctggcaaaagaggcttcaatcccgatcattaacggactctcggatctgtaccaccctatccaaatcttggccgactacctgaccctgcaagaacactacagctccctgaagggcctgactctttcctggattggcgatggaaacaacattctccattctattatgatgtccgccgccaagttcggcatgcaccttcaagccgccaccccgaagggctacgaacctgacgcctccgtgactaagctagccgaacagtacgctaaggagaacggcactaagcttctccttaccaacgatccgctggaggcggcccatggcggaaatgtgcttatcaccgacacctggattagcatggggcaggaagaagagaagaagaaacggctccaggcattccagggctaccaggtcaccatgaaaactgccaaggtcgccgctagcgactggaccttcctccactgtctgcctcgcaagcctgaagaagtggacgacgaggtgttctactccccgcgctccctcgtgtttcctgaggccgagaacagaaagtggaccatcatggccgtgatggtgtcattacttacggactacagcccgcagctgcagaagccgaagttctag

SEQ ID NO: 153 - DNA, 1065 bp, Artificial Sequence
  Synthetic construct
atgctttttaacttgagaatccttctgaacaacgccgctttccgcaacggtcataacttcatggtccggaacttcagatgtggccagcccctccaaaacaaagtgcagctgaagggccgggaccttcttacgctgaagaatttcaccggcgaagaaatcaagtacatgctctggctgtccgccgatcttaagttccgcattaagcagaagggggaatacctcccgctgctgcaagggaagtcgctgggcatgatttttgagaagcggtcaactcgcacccgcctgtccactgaaactggattcgcactgctcggtggccatccctgcttcctgaccacccaagacatccacctcggcgtgaacgagtccctgactgacaccgcccgggtcttatcctcgatggccgatgctgtgcttgcgagggtgtacaagcagtccgacctcgacacactcgcgaaggaggcctccatccccatcatcaacggcctgtccgacctttaccacccaattcagatcctcgccgattacctgaccctgcaagagcactactcgtcgctcaaggggcttaccctctcgtggattggcgacggcaacaacatccttcactccatcatgatgtcggcagcgaagttcggcatgcatctgcaagccgccacgcctaagggttatgaaccggatgcctcagtgaccaagctcgccgaacagtacgcgaaagagaatggaaccaagctacttctgaccaacgaccccctggaggccgctcacggcggcaacgtcctcattaccgatacttggatttcgatgggacaggaagaggaaaagaagaagagactgcaggcgttccagggataccaggtcaccatgaaaactgccaaagtggcagcctccgactggaccttccttcactgcctgccgaggaagcctgaagaggtggacgacgaggtgttctactccccgcgctccttggtgtttcctgaggccgaaaaccggaagtggactatcatggccgtgatggtgtccctcctcaccgactactcgccgcaactgcagaagcctaagttctag

SEQ ID NO: 154 - DNA, 1065 bp, Artificial Sequence
  Synthetic construct
atgttattcaaccttagaattctccttaacaacgccgccttccggaatgggcataactttatggtccgcaatttccgctgtggacagcctctgcaaaacaaggtccagctcaagggccgggatctgctgactctcaagaacttcactggggaagaaatcaagtacatgctctggctgagcgccgacctcaagttccgcatcaagcagaagggagagtacctcccgctgctccaagggaagtccctgggcatgatcttcgagaagagatccacccgcaccagactttccactgagactggcttcgccttgctgggaggccacccatgcttcctgacgacccaggacattcaccttggcgtgaacgagtccctgactgacaccgcaagggtgttgtcctcgatggccgacgccgtgcttgcccgggtgtacaagcagagcgatcttgacaccctggctaaggaagcttccattcccatcatcaacggtctgagcgacctgtaccacccgattcagatcctggcggactacctaaccctgcaagagcactatagctccctgaagggcctcacactttcatggatcggcgacggcaacaacatcctgcactctattatgatgagcgctgccaaattcggcatgcacctccaagccgccacgcctaaaggctacgagcccgacgcctcggtgaccaagcttgcggagcagtacgcgaaggaaaacggcaccaagctgcttctcaccaacgatcctctggaagcggcccatggtggcaacgtgctcattaccgacacttggatctccatgggacaggaggaggaaaagaagaagcggctccaggcgtttcagggttaccaggtcaccatgaaaaccgccaaggtcgcagcctccgactggaccttccttcattgccttccgcgcaagcccgaagaagtggacgatgaagtgttttactcacctcggtcactcgtgttcccggaagcagagaacaggaaatggaccattatggccgtgatggtgtccctgctcaccgattacagtccgcaactgcagaagcccaagttctag

SEQ ID NO: 155 - DNA, 1065 bp, Artificial Sequence
  Synthetic construct
atgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcaaatcctggccgattacctcaccctgcaagaacactacagctccctgaagggtctgacattgtcctggatcggcgacggcaacaacattctccattccatcatgatgtccgccgcaaaattcggcatgcatcttcaagccgccacgcctaagggttacgaacccgacgcttccgtgactaagctcgccgagcagtacgctaaggagaacggaaccaagctgctgctgactaacgacccgctagaagcagcccacgggggcaacgtgcttattactgacacctggatctccatgggccaggaggaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcacttctcaccgactacagcccgcagcttcagaagcccaagttctag

SEQ ID NO: 156 - DNA, 1065 bp, Artificial Sequence
  Synthetic construct
atgcttttcaatctccgcatcctccttaacaacgccgcgtttagaaacggccacaacttcatggtccggaacttcagatgtggccagccgcttcaaaacaaggtccagcttaagggccgggatctcctcacccttaaaaacttcaccggcgaagagatcaagtacatgctctggctctccgcggaccttaagttccgcattaagcagaagggggaataccttccgctgcttcaaggaaagagcctcggcatgatctttgagaagcgctcaaccaggaccaggctttctactgaaactgggttcgcgcttctcggcggtcatccctgcttcctcacgacccaagacatccacctcggagtgaacgaatccctcacggatactgcccgcgtgctttcgagcatggcagacgccgtgctcgcccgggtgtacaaacagtccgatctcgacactctcgccaaggaggcgtcaattcctattatcaacggtcttagtgacctttaccacccgatccagatcctcgccgattacctcacactccaagaacactacagctcccttaagggtcttaccctctcctggatcggcgacggcaacaacattctccactccatcatgatgtccgccgcaaagttcggcatgcatcttcaagccgccaccccgaagggctacgagcctgatgcttccgtgactaagctcgccgagcagtacgctaaggagaacggaaccaagcttcttctcactaacgacccactcgaagcagcccatgggggcaacgtgcttatcactgacacctggatctccatgggccaggaagaagagaagaagaagcggctccaggcgttccagggatatcaggtcaccatgaaaaccgccaaggtcgctgcctccgactggacctttctccactgcctccctcgcaaacctgaagaagtggacgacgaggtgttctactcgccccggagcctcgtgttccccgaggccgagaatagaaagtggaccattatggccgtgatggtgtcactcctcaccgactacagcccgcagcttcagaagcccaagttctag

SEQ ID NO: 157 - DNA, 1065 bp, Artificial Sequence
  Synthetic construct
atgcttttcaatctccgcatcctccttaacaacgccgcgtttagaaacggacataacttcatggtccggaacttcagatgtggacagccgcttcaaaacaaggtccagctgaagggtcgggatcttctgaccctgaagaactttaccggagaagagatcaagtacatgctctggctctccgcggacttgaagttccgcattaagcagaagggagaatacctcccgctgcttcaaggaaagagcctcggaatgatttttgagaagcgctcaaccaggacccgcctttctactgaaactggattcgcgctgctgggtggacacccctgcttcctgacgacccaggacatccacctcggagtgaacgaatccctcactgataccgcccgggtgttatcgagcatggcagatgccgtgctggccagggtgtacaaacagtccgatctggacactctggccaaggaggcgtcaattcctatcatcaacggacttagtgacctctaccatccgattcaaatcctggccgactacctcaccctgcaagaacactacagctccctgaagggtctgacattgtcctggatcggagatggaaacaacattctccactccatcatgatgtccgccgcaaaattcggaatgcatcttcaagccgccacgcctaagggttacgaacccgacgcttccgtgactaagctcgccgagcagtacgctaaggagaacggtaccaagcttctcctgaccaacgacccactagaagcagcccacggtggaaacgtgcttattactgacacttggatctccatgggacaggaggaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccgcggagcctcgtgttccccgaggccgagaatagaaagtggaccatcatggccgtgatggtgtcactgctcaccgactacagcccgcagcttcagaagcccaagttctag

SEQ ID NO: 158 - DNA, 1065 bp, Artificial Sequence
  Synthetic construct
atgcttttcaacctccgcattctcctcaacaacgctgccttccggaatggacataacttcatggtccggaacttcagatgcggacagccgcttcagaacaaggtccagcttaaggggagagatctccttaccctcaaaaacttcactggcgaagaaatcaagtacatgctctggcttagtgcggatctcaagttccgcatcaagcagaagggagaatacctcccgctccttcaaggaaagagcctcggcatgatttttgagaagaggtccaccagaactcgcctttcaaccgagactgggttcgccctgcttggcggtcacccctgcttcctcactacccaagacatccacctcggcgtgaacgagagccttaccgacaccgcccgcgtgctctcctcaatggccgacgctgtgctcgcccgggtgtacaagcagtccgaccttgatactctcgccaaggaggcctccatcccaattatcaacgggctctctgatctctaccaccctatccaaatcctcgcggactacctcaccctccaagagcactatagctcgctcaagggcctcaccctttcctggattggcgacggcaacaacattcttcactcgatcatgatgtccgccgccaagttcggcatgcatctccaagccgcgacccccaagggctacgagcctgacgcatccgtgaccaagctcgccgagcagtacgcgaaggaaaatggcaccaagcttcttctcaccaacgacccccttgaggccgctcatggcggcaacgtgctcatcactgacacttggatcagcatgggccaggaggaggaaaagaagaagcgccttcaggcattccagggttaccaggtcaccatgaaaaccgccaaagtggccgcctccgactggacctttcttcactgtctcccgcggaagcctgaagaagtggatgacgaagtgttttactcccctcggtcactcgtgttcccggaagcagaaaacaggaagtggaccattatggcggtcatggtgtccctcctcaccgactacagcccgcagcttcagaaacccaagttctag

SEQ ID NO: 159 - DNA, 1065 bp, Artificial Sequence
  Synthetic construct
atgcttttcaatctccgcatcctccttaacaacgcagcgtttagaaacggtcacaacttcatggtccggaacttccgctgtggacagccgcttcaaaacaaggtccagctgaagggtcgggaccttctgaccctgaagaactttactggagaagagatcaagtacatgctttggctgtccgcggacttgaagttccgcattaagcagaagggagaataccttccgctgctccaaggaaagagcctgggaatgatctttgagaagcgctcaaccaggacccgcctttctactgaaactggattcgcgctgctgggtggtcacccttgcttcctgacgacccaggacattcacctcggagtgaacgagtccctcactgataccgccagagtgttatcgagcatggcagatgccgtgctggctagggtgtacaaacagtccgatctggacaccctggccaaggaggcatcaattcctattatcaacggacttagtgacctctaccatccgattcaaatcctggccgattacctcaccctgcaagaacactacagctccctgaagggtctgacattgtcctggatcggagatggaaacaacattctccattccatcatgatgtccgcggccaagttcggaatgcatctccaagccgccacgccgaaaggatacgagccggacgcttccgtgactaagctcgccgagcagtacgctaaggagaacggaaccaagcttctgctgactaacgacccgctagaagccgcccacggtggaaacgtgcttattactgacacctggatctccatgggacaggaagaagagaaaaagaagcggctgcaggcgttccagggatatcaggtcaccatgaaaaccgccaaggtcgccgcctccgactggaccttccttcactgcctgcctcggaagcctgaagaagtggacgacgaggtgttctactcgccgcggagcctcgtgttccctgaggccgagaatagaaagtggaccatcatggccgtgatggtgtcactcctcaccgactacagcccgcagcttcagaagcctaagttctag

SEQ ID NO: 160 - DNA, 1065 bp, Artificial Sequence
  Synthetic construct
atgcttttcaatctccgcattctcctcaacaacgcagcctttagaaacggccacaacttcatggtccggaacttcagatgtggccagccgcttcagaacaaggtccagctcaagggccgggacctcctcaccctcaaaaactttaccggcgaagagatcaagtacatgctctggctttcggccgaccttaagttccgcatcaagcagaagggggaataccttccgctgcttcaaggaaagtccctcggcatgatctttgaaaagcgctcgaccaggacccgcctttccactgaaaccgggttcgcgcttctcggtggccacccctgcttcctcaccacccaagacattcacctcggagtgaacgaatcccttaccgataccgcaagagtgctttcgtcgatggccgatgccgtgcttgcgcgggtgtacaagcagtcagatctcgacactctcgccaaggaggcgtccattcctattatcaacggcctttccgacctttaccacccgattcagatcctcgccgattacctcaccctgcaagagcactactcgtcactcaagggtcttaccctctcctggatcggcgacggaaacaacatcctccattcgatcatgatgtccgccgccaaattcggcatgcacctccaagccgcgaccccgaagggttacgagcccgacgcttccgtgaccaagctcgccgaacagtacgctaaggaaaacggcaccaagctcctcctcactaacgaccctctcgaagcagcccatgggggcaacgtgctcattactgacacttggatctcgatgggccaggaagaggagaaaaagaagcggcttcaggcgttccagggatatcaggtcaccatgaaaaccgccaaggtcgctgcctcggactggaccttccttcactgccttccgcgcaagcctgaagaggtggacgatgaggtgttctactccccacggtcccttgtgttccccgaggccgagaataggaagtggaccatcatggccgtgatggtgtcgctcctcactgactactccccgcaacttcagaagcctaagttctag

SEQ ID NO: 161 - DNA, 1065 bp, Artificial Sequence
  Synthetic construct
atgctgtttaatctgagaatacttctaaacaacgccgccttccggaatggccataactttatggttcggaatttccgctgcggccagccgctgcagaacaaggtccagctgaagggaagagacttgctgaccctcaagaacttcaccggagaagaaatcaagtatatgctgtggctgtccgccgacctgaaattccgcatcaagcagaagggcgaatatctgccgctgttgcaagggaagtccctggggatgatcttcgagaagaggtccaccagaacacggctttcaaccgaaaccgggtttgcactgctgggtggacacccctgttttctgaccactcaagatatccacctgggcgtgaacgagtcccttaccgacactgctagggtgttgtccagcatggccgatgccgtcctggctcgcgtgtacaagcagtccgacctggataccctggcaaaggaagcgtccattcccattatcaacgggctgtccgacctgtaccatccgattcaaatcctggcggactacctgactctgcaagagcattacagcagcttgaaggggcttactctctcgtggatcggcgacgggaacaacatcctgcactccatcatgatgtccgccgccaagttcgggatgcatttgcaagctgcgaccccgaaaggttacgagcccgatgctagcgtaactaagcttgccgaacagtacgccaaagagaatggtacaaaactgcttctgactaacgacccgctggaagcagcccacggcgggaacgtgctgataaccgacacctggatttcaatggggcaggaggaagagaagaagaagcgactgcaggcgttccaaggctatcaggttaccatgaaaaccgccaaagtggcagccagcgattggactttcctgcactgtctgccgcggaagcccgaggaagttgatgacgaagtattctactcaccccggagcctcgtgttccccgaggccgaaaaccggaagtggactattatggccgtgatggtgtcgctgttgaccgactacagcccgcaactgcagaagccgaagttttag

SEQ ID NO: 162 - DNA, 1065 bp, Artificial Sequence
  Synthetic construct
atgcttttcaacctgaggatccttttgaacaacgccgcctttcgcaacggccacaactttatggtccgcaatttccgctgcgggcagccgctgcagaacaaggtccagctgaagggccgggatctgctgaccctgaagaacttcaccggggaggaaatcaagtacatgctttggctctccgccgatctgaagttcagaatcaagcagaagggagagtacctcccgttgctgcaaggaaagtcactcggaatgattttcgaaaagagaagcactaggacccgcctctcaactgaaaccgggttcgcgctgctcgggggccatccgtgtttcctgactacccaagacatccacctgggagtgaacgagtcgctgaccgacaccgcacgcgtgctgtcatccatggcggacgcagtgcttgcccgggtgtacaagcagtcggacctggacactcttgccaaggaggcatcaatccccatcattaacggactgtccgatctctaccacccgattcagatcctggctgactacctaaccctgcaagagcactactcaagcctgaaggggctgaccctgtcgtggatcggggacggcaacaacattctgcactccatcatgatgtcggcggctaagttcgggatgcatttgcaagcggcaactccgaagggttatgaacccgacgcctccgtgaccaagctggccgaacagtacgccaaggaaaacggaaccaagttgctgctgactaatgatcccctggaggcggcccacggggggaacgtgctgataaccgatacctggatctccatggggcaggaagaagagaagaaaaagcggctgcaggcattccagggataccaggtcaccatgaaaaccgcaaaagtggcagccagcgactggactttcctccattgcctgccgcgaaagccggaggaggtcgatgacgaggtgttctactccccgcggtcgctggtgttcccggaggcggaaaaccggaagtggaccattatggccgtgatggtgtcactcctgactgactacagcccgcaactgcagaagccgaagttctag

SEQ ID NO: 163 - DNA, 1074 bp, Artificial Sequence
  Synthetic construct
atgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaa

SEQ ID NO: 164 - DNA, 1073 bp, Artificial Sequence
  Synthetic construct
atgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaataagtga

SEQ ID NO: 165 - DNA, 1073 bp, Artificial Sequence; identical to SEQ ID NO: 164
  Synthetic construct

SEQ ID NO: 166 - DNA, 1074 bp, Artificial Sequence; identical to SEQ ID NO: 163
  Synthetic construct

SEQ ID NO: 167 - DNA, 1073 bp, Artificial Sequence
  Synthetic construct
atgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcatccgtgaccaagttggcggaacagtacgcgaaggagaacggaaccaagctcctgctgactaacgacccgctcgaggctgcgcatgggggtaacgtgctgattacggacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctagataagtga

SEQ ID NO: 168 - DNA, 1073 bp, Artificial Sequence; identical to SEQ ID NO: 164
  Synthetic construct

SEQ ID NO: 169 - DNA, 1074 bp, Artificial Sequence; identical to SEQ ID NO: 163
  Synthetic construct

SEQ ID NO: 170 - DNA, 1073 bp, Artificial Sequence; identical to SEQ ID NO: 164
  Synthetic construct

SEQ ID NO: 171 - DNA, 1073 bp, Artificial Sequence; identical to SEQ ID NO: 167
  Synthetic construct

SEQ ID NO: 172 - DNA, 1074 bp, Artificial Sequence; identical to SEQ ID NO: 163
  Synthetic construct

SEQ ID NO: 173 - DNA, 1073 bp, Artificial Sequence; identical to SEQ ID NO: 164
  Synthetic construct

SEQ ID NO: 174 - DNA, 1073 bp, Artificial Sequence; identical to SEQ ID NO: 167
  Synthetic construct

SEQ ID NO: 175 - DNA, 1065 bp, Artificial Sequence
  Synthetic construct
atgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 176 - DNA, 1065 bp, Artificial Sequence; identical to SEQ ID NO: 175
  Synthetic construct

SEQ ID NO: 177 - DNA, 1065 bp, Artificial Sequence; identical to SEQ ID NO: 175
  Synthetic construct

SEQ ID NO: 178 - DNA, 1074 bp, Artificial Sequence
  Synthetic construct
atgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaataagtgaa

SEQ ID NO: 179 - DNA, 1077 bp, Artificial Sequence
  Synthetic construct
atgggcgtcttcaacctgcggatcctgctgaacaacgccgccttccggaacggccacaacttcatggtccgcaacttcagatgcggccagcccctgcagaacaaggtgcagctgaagggccgggacctgctgaccctgaagaacttcaccggcgaagagatcaagtacatgctgtggctgagcgccgacctgaagttccggatcaagcagaagggcgagtacctgcccctgctgcaaggcaagagcctgggcatgatcttcgagaagcggagcacccggacccggctgagcaccgagacaggctttgccctgctgggaggccacccctgctttctgaccacccaggacatccacctgggcgtgaacgagagcctgaccgacaccgccagagtgctgagcagcatggccgacgccgtgctggcccgggtgtacaagcagagcgacctggacaccctggccaaagaggccagcatccccatcatcaacggcctgagcgacctgtaccaccccatccagatcctggccgactacctgaccctgcaggaacactacagctccctgaagggcctgaccctgagctggatcggcgacggcaacaacatcctgcacagcatcatgatgagcgccgccaagttcggcatgcatctgcaggccgccacccccaagggctacgagcctgatgccagcgtgaccaagctggccgagcagtacgccaaagagaacggcaccaagctgctgctgaccaacgaccccctggaagccgcccacggcggcaacgtgctgatcaccgacacctggatcagcatgggccaggaagaggaaaagaagaagcggctgcaggccttccagggctaccaggtcacaatgaagaccgccaaggtggccgccagcgactggaccttcctgcactgcctgccccggaagcccgaagaggtggacgacgaggtgttctacagcccccggtccctggtgttccccgaggccgagaaccggaagtggaccattatggccgtgatggtgtccctgctgaccgactactccccccagctgcagaagcccaagttctagataagtgaa

SEQ ID NO: 180 - DNA, 1077 bp, Artificial Sequence
  Synthetic construct
atgggcgtcttcaacctgcggatcctgctgaacaacgccgccttccggaacggccacaacttcatggtccgcaacttcagatgcggccagcccctgcagaacagggtgcagctgaagggccgggacctgctgaccctgaagaacttcaccggcgaagagatcaggtacatgctgtggctgagcgccgacctgaagttccggatcaagcagaagggcgagtacctgcccctgctgcaaggcaagagcctgggcatgatcttcgagaagcggagcacccggacccggctgagcaccgagacaggctttgccctgctgggaggccacccctgctttctgaccacccaggacatccacctgggcgtgaacgagagcctgaccgacaccgccagagtgctgagcagcatggccgacgccgtgctggcccgggtgtacaagcagagcgacctggacaccctggccaaagaggccagcatccccatcatcaacggcctgagcgacctgtaccaccccatccagatcctggccgactacctgaccctgcaggaacactacagctccctgaagggcctgaccctgagctggatcggcgacggcaacaacatcctgcacagcatcatgatgagcgccgccaagttcggcatgcatctgcaggccgccacccccaagggctacgagcctgatgccagcgtgaccaagctggccgagcagtacgccaaagagaacggcaccaagctgctgctgaccaacgaccccctggaagccgcccacggcggcaacgtgctgatcaccgacacctggatcagcatgggccaggaagaggaaaagaagaagcggctgcaggccttccagggctaccaggtcacaatgaagaccgccaaggtggccgccagcgactggaccttcctgcactgcctgccccggaagcccgaagaggtggacgacgaggtgttctacagcccccggtccctggtgttccccgaggccgagaaccggaagtggaccattatggccgtgatggtgtccctgctgaccgactactccccccagctgcagaagcccaagttctagataagtgaa

SEQ ID NO: 181 - DNA, 1077 bp, Artificial Sequence
  Synthetic construct
atgctggtcttcaacctgcggatcctgctgaacaacgccgccttccggaacggccacaacttcatggtccgcaacttcagatgcggccagcccctgcagaacagggtgcagctgaagggccgggacctgctgaccctgaagaacttcaccggcgaagagatcaggtacatgctgtggctgagcgccgacctgaagttccggatcaagcagaagggcgagtacctgcccctgctgcaaggcaagagcctgggcatgatcttcgagaagcggagcacccggacccggctgagcaccgagacaggctttgccctgctgggaggccacccctgctttctgaccacccaggacatccacctgggcgtgaacgagagcctgaccgacaccgccagagtgctgagcagcatggccgacgccgtgctggcccgggtgtacaagcagagcgacctggacaccctggccaaagaggccagcatccccatcatcaacggcctgagcgacctgtaccaccccatccagatcctggccgactacctgaccctgcaggaacactacagctccctgaagggcctgaccctgagctggatcggcgacggcaacaacatcctgcacagcatcatgatgagcgccgccaagttcggcatgcatctgcaggccgccacccccaagggctacgagcctgatgccagcgtgaccaagctggccgagcagtacgccaaagagaacggcaccaagctgctgctgaccaacgaccccctggaagccgcccacggcggcaacgtgctgatcaccgacacctggatcagcatgggccaggaagaggaaaagaagaagcggctgcaggccttccagggctaccaggtcacaatgaagaccgccaaggtggccgccagcgactggaccttcctgcactgcctgccccggaagcccgaagaggtggacgacgaggtgttctacagcccccggtccctggtgttccccgaggccgagaaccggaagtggaccattatggccgtgatggtgtccctgctgaccgactactccccccagctgcagaagcccaagttctagataagtgaa

SEQ ID NO: 182 - DNA, 1074 bp, Artificial Sequence
  Synthetic construct
atgctgttcaacctgaggatcctgctgaacaacgcagctttcaggaacggccacaacttcatggtgaggaacttccggtgcggccagcccctgcagaacaaggtgcagctgaagggcagggacctgctgaccctgaagaacttcaccggagaggagatcaagtacatgctgtggctgagcgcagacctgaagttcaggatcaagcagaagggagagtacctgcccctgctgcaggggaagtccctgggcatgatcttcgagaagaggagtaccaggaccaggctgagcaccgaaaccggcttcgccctgctgggaggacacccctgcttcctgaccacccaggacatccacctgggcgtgaacgagagtctgaccgacaccgccagggtgctgtctagcatggccgacgccgtgctggccagggtgtacaagcagtcagacctggacaccctggctaaggaggccagcatccccatcatcaacggcctgagcgacctgtaccaccccatccagatcctggctgactacctgaccctgcaggagcactacagctctctgaagggcctgaccctgagctggatcggcgacgggaacaacatcctgcacagcatcatgatgagcgccgccaagttcggcatgcacctgcaggccgctacccccaagggttacgagcccgacgccagcgtgaccaagctggcagagcagtacgccaaggagaacggcaccaagctgctgctgaccaacgaccccctggaggccgcccacggaggcaacgtgctgatcaccgacacctggatcagcatgggacaggaggaggagaagaagaagcggctgcaggctttccagggttaccaggtgaccatgaagaccgccaaggtggctgccagcgactggaccttcctgcactgcctgcccaggaagcccgaggaggtggacgacgaggtgttctactctcccaggagcctggtgttccccgaggccgagaacaggaagtggaccatcatggctgtgatggtgtccctgctgaccgactacagcccccagctgcagaagcccaagttctgaataagtgaa

SEQ ID NO: 183 - DNA, 1074 bp, Artificial Sequence
  Synthetic construct
atgctgttcaacctgaggatcctgctgaacaacgcagctttcaggaacggccacaacttcatggtgaggaacttccggtgcggccagcccctgcagaacaaggtgcagctgaagggcagggacctgctgaccctgaagaacttcaccggagaggagatcaagtacatgctgtggctgagcgcagacctgaagttcaggatcaagcagaagggagagtacctgcccctgctgcaggggaagtccctgggcatgatcttcgagaagaggagtaccaggaccaggctgagcaccgaaaccggcttcgccctgctgggaggacacccctgcttcctgacgacccaggacatccacctcggagtgaacgaatccctcaccgataccgcccgggtgttatcaagcatggcagatgccgtgctggccagggtgtacaaacagtccgatctggacactctggctaaggaggccagcatccccatcatcaacggcctgagcgacctgtaccaccccatccagatcctggctgactacctgaccctgcaggagcactacagctctctgaagggcctgaccctgagctggatcggcgacgggaacaacatcctgcactccatcatgatgtccgccgcgaagttcggaatgcatctgcaagccgccacgccaaaaggatacgaaccggatgcgcccgtgacaaagttggcggaacagtacgctaaggagaacggaaccaagctgctgctgaccaacgaccccctggaggccgcccacggaggcaacgtgctgatcaccgacacctggatcagcatgggacaggaggaggagaagaagaagcggctgcaggctttccagggttaccaggtgaccatgaagaccgccaaggtggctgccagcgactggaccttcctgcactgcctgcccaggaagcccgaggaggtggacgacgaggtgttctactctcccaggagcctggtgttccccgaggccgagaacaggaagtggaccatcatggctgtgatggtgtccctgctgaccgactacagcccccagctgcagaagcccaagttctgaataagtgaa

SEQ ID NO: 184 - DNA, 1074 bp, Artificial Sequence; identical to SEQ ID NO: 178
  Synthetic construct

SEQ ID NO: 185 - DNA, 1074 bp, Artificial Sequence; identical to SEQ ID NO: 178
  Synthetic construct

SEQ ID NO: 186 - DNA, 1065 bp, Artificial Sequence
  Synthetic construct
atgcttttcaacttgagaatcctgctgaacaacgccgcctttcgcaacggtcacaattttatggtcagaaacttcagatgcggacagcccctccaaaacaaggtccagctgaagggccgcgatctcctcaccctgaagaacttcacgggggaggagatcaagtacatgctgtggctctccgctgacctgaagttcaggatcaagcagaagggagaatatctgccgctgctgcaagggaagtccctggggatgattttcgagaagcggagcacccggactcggctctccactgaaactggtttcgcccttctgggcggtcacccctgcttcctgaccactcaagacattcacctcggagtgaacgagtccttgactgacaccgcccgggtgctgtcgagcatggcagacgccgtgctagcccgcgtgtacaagcagtcagacctcgataccctggccaaggaggcttcgatcccgatcatcaacgggttgtccgacctgtaccacccgattcagattctcgccgactacctcaccctgcaagagcattacagctccctgaaggggcttaccctgtcctggattggcgacggaaacaacatcctgcactccattatgatgtcggcggccaagttcggcatgcacctccaagccgcgacccctaagggttacgaaccagacgcgtcagtgactaagctggccgaacagtacgcaaaggaaaatggcacgaagctgctcctgaccaacgatccgttggaagccgcccatggcggaaatgtgctcatcaccgacacctggatctcgatgggacaggaggaagagaagaagaagcggctgcaggcgttccagggctaccaggtcaccatgaaaactgccaaggtggccgccagcgactggaccttcctgcactgccttccgcgcaagcctgaggaggtggacgatgaagtgttctactctccacggtccctggtgttccccgaggcggagaaccgcaaatggaccatcatggctgtgatggtcagcctgctgaccgattacagccctcagttgcaaaagccgaagttttga

SEQ ID NO: 187 - DNA, 1065 bp, Artificial Sequence
  Synthetic construct
atgctgttcaacctccgcatcctcctcaacaacgccgcattcagaaacgggcacaacttcatggtcagaaacttccgctgcgggcaacccctacaaaacaaggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctga

SEQ ID NO: 188 - DNA, 1074 bp, Artificial Sequence
  Synthetic construct
atgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctgaataagtaga

SEQ ID NO: 189 - DNA, 1074 bp, Artificial Sequence
  Synthetic construct
atgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcatccgtgaccaagttggcggaacagtacgcgaaggagaacggaaccaagctcctgctgactaacgacccgctcgaggctgcgcatgggggtaacgtgctgattacggacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctgaataagtaga

SEQ ID NO: 190 - DNA, 1074 bp, Artificial Sequence
  Synthetic construct
atgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaataagtaga

SEQ ID NO: 191 - DNA, 1065 bp, Artificial Sequence; identical to SEQ ID NO: 136
  Synthetic construct

SEQ ID NO: 192 - DNA, 1065 bp, Artificial Sequence; identical to SEQ ID NO: 136
  Synthetic construct

SEQ ID NO: 193 - DNA, 1065 bp, Artificial Sequence; identical to SEQ ID NO: 136
  Synthetic construct

SEQ ID NO: 194 - DNA, 1065 bp, Artificial Sequence; identical to SEQ ID NO: 136
  Synthetic construct

SEQ ID NO: 195 - DNA, 1065 bp, Artificial Sequence; identical to SEQ ID NO: 136
  Synthetic construct

SEQ ID NO: 196 - DNA, 1065 bp, Artificial Sequence
  Synthetic construct
atggtgttcaacctccgcatcctcctcaacaacgccgcattcagaaacgggcacaacttcatggtcagaaacttccgctgcgggcaacccctacaaaacaaggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctga

SEQ ID NO: 197 - DNA, 1065 bp, Artificial Sequence
  Synthetic construct
atggtgttcaacctccgcatcctcctcaacaacgccgcattcagaaacgggcacaacttcatggtcagaaacttccgctgcgggcaacccctacaaaaccgggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctga

SEQ ID NO: 198 - DNA, 1065 bp, Artificial Sequence
  Synthetic construct
atggtgttcaacctccgcatcctcctcaacaacgccgcattcagaaacgggcacaacttcatggtcagaaacttccgctgcgggcaacccctacaaaaccgggtccagctcaaggggcgggacctcctgaccctgaagaacttcaccggcgaagagatccggtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctga

SEQ ID NO: 199 - DNA, 1065 bp, Artificial Sequence
  Synthetic construct
atgctggtcaacctccgcatcctcctcaacaacgccgcattcagaaacgggcacaacttcatggtcagaaacttccgctgcgggcaacccctacaaaacaaggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctga

SEQ ID NO: 200 - DNA, 1065 bp, Artificial Sequence
  Synthetic construct
atgctggtcaacctccgcatcctcctcaacaacgccgcattcagaaacgggcacaacttcatggtcagaaacttccgctgcgggcaacccctacaaaaccgggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctga

SEQ ID NO: 201 - DNA, 1065 bp, Artificial Sequence
  Synthetic construct
atgctggtcaacctccgcatcctcctcaacaacgccgcattcagaaacgggcacaacttcatggtcagaaacttccgctgcgggcaacccctacaaaaccgggtccagctcaaggggcgggacctcctgaccctgaagaacttcaccggcgaagagatccggtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctga

SEQ ID NO: 202 - DNA, 1065 bp, Artificial Sequence
  Synthetic construct
atgctggtcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 203 - DNA, 1065 bp, Artificial Sequence
  Synthetic construct
atgctggtcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 204 - DNA, 1065 bp, Artificial Sequence
  Synthetic construct
atgcttgtcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctag

SEQ ID NO: 205 - DNA, 1065 bp, Artificial Sequence
  Synthetic construct
atgcttgtcaatctccgcatcctccttaacaacgccgcgtttagaaacggccacaacttcatggtccggaacttcagatgtggccagccgcttcaaaaccgggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctag

SEQ ID NO: 206 - DNA, 1068 bp, Artificial Sequence
  Synthetic construct
atgggccttgtcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctag

SEQ ID NO: 207 - DNA, 1068 bp, Artificial Sequence
  Synthetic construct
atgggccttgtcaatctccgcatcctccttaacaacgccgcgtttagaaacggccacaacttcatggtccggaacttcagatgtggccagccgcttcaaaaccgggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctag

SEQ ID NO: 208 - DNA, 1071 bp, Artificial Sequence
  Synthetic construct
atgggcggacttgtcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctag

SEQ ID NO: 209 - DNA, 1077 bp, Artificial Sequence
  Synthetic construct
atggcccttttcaatctccgcatcctccttaacaacgccgcgtttagaaacggccacaacttcatggtccggaacttcagatgtggccagccgcttcaaggcaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaa

SEQ ID NO: 210 - DNA, 1077 bp, Artificial Sequence
  Synthetic construct
atggcccttttcaatctccgcatcctccttaacaacgccgcgtttagaaacggccacaacttcatggtccggaacttcagatgtggccagccgcttcaaggccgggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaa

SEQ ID NO: 211 - DNA, 1077 bp, Artificial Sequence
  Synthetic construct
atggcccttttcaatctccgcatcctccttaacaacgccgcgtttagaaacggccacaacttcatggtccggaacttcagatgtggccagccgcttcaaggccgggtccagctgaagggccgggatcttctgaccctgaagaactttactggcgaagagatcaggtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaa

SEQ ID NO: 212 - DNA, 1077 bp, Artificial Sequence
  Synthetic construct
atggcccttgtcaatctccgcatcctccttaacaacgccgcgtttagaaacggccacaacttcatggtccggaacttcagatgtggccagccgcttcaaggcagggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctaagtgaataga

SEQ ID NO: 213 - DNA, 1077 bp, Artificial Sequence
  Synthetic construct
atggcccttttcaatctccgcatcctccttaacaacgccgcgtttagaaacggccacaacttcatggtccggaacttcagatgtggccagccgcttcaagtcaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaa

SEQ ID NO: 214 - DNA, 1077 bp, Artificial Sequence
  Synthetic construct
atggcccttttcaatctccgcatcctccttaacaacgccgcgtttagaaacggccacaacttcatggtccggaacttcagatgtggccagccgcttcaagtcagggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaa

SEQ ID NO: 215 - DNA, 1065 bp, Artificial Sequence; identical to SEQ ID NO: 175
  Synthetic construct

SEQ ID NO: 216 - DNA, 1068 bp, Artificial Sequence
  Synthetic construct
atgggagtattcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 217 - DNA, 1068 bp, Artificial Sequence
  Synthetic construct
atgggagtattcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 218 - DNA, 1068 bp, Artificial Sequence
  Synthetic construct
atgctggtattcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 219 - DNA, 1068 bp, Artificial Sequence; identical to SEQ ID NO: 216
  Synthetic construct

SEQ ID NO: 220 - DNA, 1068 bp, Artificial Sequence; identical to SEQ ID NO: 217
  Synthetic construct

SEQ ID NO: 221 - DNA, 1068 bp, Artificial Sequence; identical to SEQ ID NO: 218
  Synthetic construct

SEQ ID NO: 222 - DNA, 1073 bp, Artificial Sequence
  Synthetic construct
atgttgttcaacttgaggatcttgttgaacaacgccgccttcaggaacggacacaacttcatggtaaggaacttcaggtgcggacagcccttgcagaacaaagtacagttgaaaggaagggacttgttgacattgaaaaacttcacaggagaagaaatcaaatacatgttgtggttgtcggccgacttgaaattcaggatcaaacagaaaggagaatacttgcccttgttgcagggaaaatcgttgggaatgatcttcgaaaaaaggtcgacaaggacaaggttgtcgacagaaacaggattcgccttgttgggaggacacccctgcttcttgacaacacaggacatccacttgggagtaaacgaatcgttgacagacacagccagggtattgtcgtcgatggccgacgccgtattggccagggtatacaaacagtcggacttggacacattggccaaagaagcctcgatccccatcatcaacggattgtcggacttgtaccaccccatccagatcttggccgactacttgacattgcaggaacactactcgtcgttgaaaggattgacattgtcgtggatcggagacggaaacaacatcttgcactcgatcatgatgtcggccgccaaattcggaatgcacttgcaggccgccacacccaaaggatacgaacccgacgcctcggtaacaaaattggccgaacagtacgccaaagaaaacggaacaaaattgttgttgacaaacgaccccttggaagccgcccacggaggaaacgtattgatcacagacacatggatctcgatgggacaggaagaagaaaaaaaaaaaaggttgcaggccttccagggataccaggtaacaatgaaaacagccaaagtagccgcctcggactggacattcttgcactgcttgcccaggaaacccgaagaagtagacgacgaagtattctactcgcccaggtcgttggtattccccgaagccgaaaacaggaaatggacaatcatggccgtaatggtatcgttgttgacagactactcgccccagttgcagaaacccaaattctgaatagtgaa

SEQ ID NO: 223 - DNA, 1065 bp, Artificial Sequence
  Synthetic construct
atgctgttcaacctgcgcatcctgctgaacaacgccgccttccgcaacggccacaacttcatggtgcgcaacttccgctgcggccagcccctgcagggcaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 224 - DNA, 1065 bp, Artificial Sequence
  Synthetic construct
atgctgttcaacctgcgcatcctgctgaacaacgccgccttccgcaacggccacaacttcatggtgcgcaacttccgctgcggccagcccctgcagggccgggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 225 - DNA, 1065 bp, Artificial Sequence
  Synthetic construct
atgctgttcaacctgcgcatcctgctgaacaacgccgccttccgcaacggccacaacttcatggtgcgcaacttccgctgcggccagcccctgcagggccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 226 - DNA, 1065 bp, Artificial Sequence; identical to SEQ ID NO: 223
  Synthetic construct

SEQ ID NO: 227 - DNA, 1065 bp, Artificial Sequence; identical to SEQ ID NO: 175
  Synthetic construct

SEQ ID NO: 228 - DNA, 1068 bp, Artificial Sequence; identical to SEQ ID NO: 218
  Synthetic construct

SEQ ID NO: 229 - DNA, 1068 bp, Artificial Sequence; identical to SEQ ID NO: 218
  Synthetic construct

SEQ ID NO: 230 - RNA, 21 nt, Artificial Sequence
  Synthetic construct
aaccaaucgaaagaaaccaaa

SEQ ID NO: 231 - RNA, 21 nt, Artificial Sequence
  Synthetic construct
cucuaaucaccaggaguaaaa

SEQ ID NO: 232 - RNA, 21 nt, Artificial Sequence
  Synthetic construct
gagagagaucuuaacaaaaaa

SEQ ID NO: 233 - RNA, 21 nt, Artificial Sequence
  Synthetic construct
uguguaacaacaacaacaaca

SEQ ID NO: 234 - RNA, 21 nt, Artificial Sequence
  Synthetic construct
ccgcaguaggaagagaaagcc

SEQ ID NO: 235 - RNA, 21 nt, Artificial Sequence
  Synthetic construct
aaaaaaaaaagaaaucauaaa

SEQ ID NO: 236 - RNA, 21 nt, Artificial Sequence
  Synthetic construct
gagagaagaaagaagaagacg

SEQ ID NO: 237 - RNA, 21 nt, Artificial Sequence
  Synthetic construct
caauuaaaaauacuuaccaaa

SEQ ID NO: 238 - RNA, 21 nt, Artificial Sequence
  Synthetic construct
gcaaacagaguaagcgaaacg

SEQ ID NO: 239 - RNA, 21 nt, Artificial Sequence
  Synthetic construct
gcgaagaagacgaacgcaaag

SEQ ID NO: 240 - RNA, 21 nt, Artificial Sequence
  Synthetic construct
uuaggacuguauugacuggcc

SEQ ID NO: 241 - RNA, 21 nt, Artificial Sequence
  Synthetic construct
aucaucggaauucggaaaaag

SEQ ID NO: 242 - RNA, 21 nt, Artificial Sequence
  Synthetic construct
aaaacaaaaguuaaagcagac

SEQ ID NO: 243 - RNA, 21 nt, Artificial Sequence
  Synthetic construct
uuuaucucaaauaagaaggca

SEQ ID NO: 244 - RNA, 21 nt, Artificial Sequence
  Synthetic construct
gguggggaggugagauuucuu

SEQ ID NO: 245 - RNA, 21 nt, Artificial Sequence
  Synthetic construct
ugauuaggaaacuacaaagcc

SEQ ID NO: 246 - RNA, 21 nt, Artificial Sequence
  Synthetic construct
cauuuuucaauuucauaaaac

SEQ ID NO: 247 - RNA, 21 nt, Artificial Sequence
  Synthetic construct
uuacuuuuaagcccaacaaaa

SEQ ID NO: 248 - RNA, 21 nt, Artificial Sequence
  Synthetic construct
ggcguguguguguguuguuga

SEQ ID NO: 249 - RNA, 21 nt, Artificial Sequence
  Synthetic construct
guggugaaggggaagguuuag

SEQ ID NO: 250 - RNA, 21 nt, Artificial Sequence
  Synthetic construct
uuguuuuuuuuugguuugguu

SEQ ID NO: 251 - RNA, 1331 nt, Artificial Sequence
  Synthetic construct
aggauuauuacaucaaaacaaaaagccgccaccaugcugguauucaaccugcgcauccugcugaacaacgccgccuuccgcaacggccacaacuucauggugcgcaacuuccgcugcggccagccccugcagaaccgggugcagcugaagggccgcgaccugcugacccugaagaacuucaccggcgaggagauccgguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggcuucgcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugaggucucuaguaaugagcuggagccucgguagccguuccuccugcccgcugggccucccaacgggcccuccuccccuccuugcaccggcccuuccuggucuuugaauaaagucugagugggcagcaucuagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 252 - RNA, 1334 nt, Artificial Sequence
  Synthetic construct
aggauuauuacaucaaaacaaaaagccgccaccaugcugguauucaaccugcgcauccugcugaacaacgccgccuuccgcaacggccacaacuucauggugcgcaacuuccgcugcggccagccccugcagaaccgggugcagcugaagggccgcgaccugcugacccugaagaacuucaccggcgaggagauccgguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggcuucgcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugaggucucuaguaaugagcuggagccucgguagccguuccuccugcccgcugggccucccaacgggcccuccuccccuccuugcaccggcccuuccuggucuuugaauaaagucugagugggcagcaucuagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagcu

SEQ ID NO: 253 - RNA, 1328 nt, Artificial Sequence
  Synthetic construct
aggauuauuacaucaaaacaaaaagccgccaccaugcuguucaaccugcgcauccugcugaacaacgccgccuuccgcaacggccacaacuucauggugcgcaacuuccgcugcggccagccccugcagaacaaggugcagcugaagggccgcgaccugcugacccugaagaacuucaccggcgaggagaucaaguacaugcuguggcugagcgccgaccugaaguuccgcaucaagcagaagggcgaguaccugccccugcugcagggcaagagccugggcaugaucuucgagaagcgcagcacccgcacccgccugagcaccgagacaggcuucgcccugcugggcggccaccccugcuuccugaccacccaggacauccaccugggcgugaacgagagccugaccgacaccgcccgcgugcugagcagcauggccgacgccgugcuggcccgcguguacaagcagagcgaccuggacacccuggccaaggaggccagcauccccaucaucaacggccugagcgaccuguaccaccccauccagauccuggccgacuaccugacccugcaggagcacuacagcagccugaagggccugacccugagcuggaucggcgacggcaacaacauccugcacagcaucaugaugagcgccgccaaguucggcaugcaccugcaggccgccacccccaagggcuacgagcccgacgccagcgugaccaagcuggccgagcaguacgccaaggagaacggcaccaagcugcugcugaccaacgacccccuggaggccgcccacggcggcaacgugcugaucaccgacaccuggaucagcaugggccaggaggaggagaagaagaagcgccugcaggccuuccagggcuaccaggugaccaugaagaccgccaagguggccgccagcgacuggaccuuccugcacugccugccccgcaagcccgaggagguggacgacgagguguucuacagcccccgcagccugguguuccccgaggccgagaaccgcaaguggaccaucauggccgugauggugagccugcugaccgacuacagcccccagcugcagaagcccaaguucugaggucucuaguaaugagcuggagccucgguagccguuccuccugcccgcugggccucccaacgggcccuccuccccuccuugcaccggcccuuccuggucuuugaauaaagucugagugggcagcaucuagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2022/0096520 A1
(43) Pub. Date: Mar. 31, 2022

(54) MODIFIED PROTEINS AND ASSOCIATED METHODS OF TREATMENT

(71) Applicant: Arcturus Therapeutics, Inc., San Diego, CA (US)

(72) Inventors: Carlos G. Perez-Garcia, San Diego, CA (US); Padmanabh Chivukula, San Diego, CA (US); Kiyoshi Tachikawa, San Diego, CA (US)

(21) Appl. No.: 17/311,271

(22) PCT Filed: Dec. 6, 2019
(86) PCT No.: PCT/US19/65046

§ 371 Date: Jun. 4, 2021

Related U.S. Application Data
(60) Provisional application No. 62/776,322, filed on Dec. 6, 2018.

(51) Int. Cl. A61K 31/7105; C12Q 1/37; C07K 14/435

(52) U.S. Cl. CPC A61K 31/7105; C07K 14/435; C12Q 1/37

(57) ABSTRACT

The present disclosure provides a modified human protein having improved in vivo stability. The modified human protein has been altered from a wild-type human protein at either at least one ubiquitination site, at the signal peptide portion, or both. The protein of the disclosure can be produced from a codon optimized mRNA suitable for administration to a patient suffering from deficiency of the wild-type protein, wherein upon administration of the mRNA to the patient, the modified protein of the disclosure is expressed in the patient in therapeutically effective amounts.

### CROSS-REFERENCE TO RELATED APPLICATIONS

This application claims priority to U.S. Provisional Application No. 62/776,322, filed Dec. 6, 2018. The content of the application is incorporated herein by reference in its entirety.

### SEQUENCE LISTING

The instant application contains a Sequence Listing which has been submitted electronically in ASCII format and is hereby incorporated by reference in its entirety. Said ASCII copy, created on Dec. 4, 2019 is named 049386_522001WO_SL.txt and is 363 kilobytes in size.

### TECHNICAL FIELD OF THE INVENTION

This invention relates to the fields of molecular biology and genetics, as well as to biopharmaceuticals and therapeutics generated from translatable molecules. More particularly, this invention relates to methods, structures and compositions for molecules having the ability to be translated into active polypeptides or proteins, for use in vivo and as therapeutics.

### BACKGROUND

Novel approaches and therapies are still needed for the treatment of diseases that are associated with proteins which are genetically deficient in amount or in function. Strategies are needed which overcome the challenges and limitations associated with, for example, gene therapy. Poor stability of therapeutic proteins, poor delivery of certain therapeutic proteins needed for normal cellular function and efficient delivery to target cells are still challenges.

### SUMMARY

The present disclosure provides modified human therapeutic protein sequences which have been altered from the wild-type sequence to remove one or more predicted ubiquitination sites. The present disclosure also provides human therapeutic proteins which have been designed to contain a stabilized signaling peptide to ensure delivery of the protein to a targeted organelle. The removal of predicted ubiquitination sites preferably comprises replacing N-terminus residues that have been found to support ubiquitination such as asparagine, arginine, leucine, lysine or phenylalanine with residues that have been found to be stabilizing against ubiquitination such as alanine, glycine, methionine, serine, threonine, valine and proline. For example, stabilization of a modified ornithine transcarbamylase (OTC) protein of SEQ ID NO: 4 in this manner is particularly advantageous for preserving the stability of the modified OTC protein during its transport from the cytosol to the mitochondria wherein it exerts its enzymatic activity.

In some embodiments, a modified protein is provided having an amino acid sequence derived from an amino acid sequence of a human wild-type protein, wherein the amino acid sequence of the human wild-type protein has been modified to remove one or more ubiquitination sites identified as being present in the amino acid sequence of the human wild-type protein but not being present in a homologous nonhuman animal wild-type protein.

In other embodiments, a modified protein is provided having an amino acid sequence derived from an amino acid sequence of a wild-type protein, the wild-type protein having a signal peptide located at a terminal portion, wherein the amino acid sequence of the signal peptide has been modified by changing an amino acid at the +1 or +2 position or by adding an amino acid at the +1 or +2 position. In some embodiments, the amino acid residue is changed to or is added as a stabilizing amino acid at the +1 or +2 position. Stabilizing amino acids can include valine, methionine, glycine, proline, threonine, alanine, and serine.

In yet other embodiments, a modified protein is provided having an amino acid sequence derived from an amino acid sequence of a human wild-type protein, the human wild-type protein having a signal peptide located at a terminal portion, wherein i) the amino acid sequence of the signal peptide has been modified by changing an amino acid at the +1 or +2 position or by adding an amino acid at the +1 or +2 position; and ii) the amino-acid sequence of the human wild-type protein has been modified to remove one or more ubiquitination sites identified as being present in the amino acid sequence of the human wild-type protein but not being present in a homologous nonhuman animal wild-type protein.

In yet other embodiments, a polynucleotide is provided comprising a sequence encoding any of the modified peptides described herein. In some embodiments, a composition is provided comprising a polynucleotide described herein and a pharmaceutically acceptable carrier. In some embodiments, the pharmaceutically acceptable carrier comprises a transfection reagent, a lipid nanoparticle, or a liposome. In some embodiments, a method is provided for ameliorating, preventing, delaying onset, or treating a disease or condition associated with a deficiency of the human wild-type protein in a subject identified as suffering from said deficiency, the method comprising administering to the subject a composition described herein.

In yet other embodiments, a method of modifying a protein of interest is provided, wherein the protein of interest is a human wild-type protein, the method comprising the steps of i) identifying ubiquitination sites in the amino acid sequence of the human wild-type protein which are not present in the amino acid sequence of a homologous nonhuman animal wild-type protein; and ii) removing at least one of the ubiquitination sites identified in step (i) from the amino acid sequence of the human wild-type protein to provide the modified protein of interest.

Preferably, the protein of SEQ ID NO: 4 described herein is produced from a nucleic acid encoding the protein of SEQ ID NO: 4. The nucleic acid may be RNA or DNA that encodes the protein of SEQ ID NO: 4. Preferably the nucleic acid is a heterologous mRNA construct comprising an open reading frame encoding for the modified protein of SEQ ID NO: 4. Preferably, the open reading frame is a codon-optimized open reading frame. Preferably, the open reading frame sequence is optimized to have a theoretical minimum of uridines possible to encode for the modified protein. Preferably, the heterologous mRNA construct comprises a 5′ cap, a 5′UTR, a 3′UTR, an open reading frame encoding a modified protein of SEQ ID NO: 4 and a 3′ poly A tail. Preferably, the 5′UTR derived from a gene expressed by Arabidopsis thaliana. Preferably the 5″ UTR derived from a gene expressed by Arabidopsis thaliana is found in Table 2.

The mRNA constructs described herein provide high-efficiency expression of the proteins described herein. The expression can be in vitro, ex vivo, or in vivo.

The present disclosure also provides pharmaceutical compositions comprising the mRNA sequences described herein and methods of treating a disease associated with a protein deficiency by administering the pharmaceutical compositions comprising the mRNA sequences described herein to a patient in need thereof wherein the deficient protein is expressed in a patient.

### BRIEF DESCRIPTION OF THE DRAWINGS

FIGS. 1A-B show scatter plots illustrating protein expression of an exemplary modified protein described herein in hepatocyte cell lines Hepa1,6 (mouse) and Hep3B (human) at 24 hours (FIG. 1A) and 48 hours (FIG. 1B) using In-Cell Western (ICW) assays.

FIGS. 2A-B show scatter plots illustrating the correlation of protein stability compounds screened in Hepa1,6 cells (FIG. 2A) and Hep3B cells (FIG. 2B) at 24 h in Round 1.

FIGS. 3A-B shows scatter plots illustrating the correlation of protein stability compounds screened in human primary hepatocytes at 24 h and 48 h in Round 2 (newly optimized compounds based on Round 1) as shown with FIG. 3A and FIG. 3B.

FIGS. 4A-B shows scatter plots illustrating the correlation of protein stability compounds screened in human primary hepatocytes at 24 h and 48 h in Round 3 (newly optimized compounds based on rounds 1 and 2) as shown with FIG. 4A and FIG. 4B.

FIG. 5 is a plot illustrating protein expression levels of an exemplary modified protein described herein in human primary hepatocytes transfected with mRNAs encoding the modified proteins. 1799.1 is an mRNA having the sequence of SEQ ID NO: 175 wherein 100% of the of the uridines in SEQ ID NO: 175 are N1-methylpseudouridine (N1MPU).

FIGS. 6A-B shows bar graphs depicting time course expression levels of an exemplary modified protein in spf/ash mice dosed at 10 mg/kg with human-specific protein mRNA epitopes (FIG. 6A) or mouse-specific protein mRNA epitopes (FIG. 6B).

FIG. 7 is a bar graph depicting expression levels of an exemplary modified protein described herein in spf/ash mice dosed at 3 mg/kg with mRNAs using two different chemistries wherein 100% of the uridines are N1-methylpseudouridine (N1MPU) and 100% of the uridines are 5-methoxyuridine (5MeOU).

FIG. 8 is a graph depicting expression levels of an exemplary modified protein described herein in Balb/c mice dosed with mRNAs at three different doses and using two different chemistries (N1MPU and 5MeOU).

FIG. 9 is a western blot illustrating the expression levels of exemplary modified proteins described herein for mRNAs in spf/ash mice dosed at 1 mg/kg and 3 mg/kg.

FIG. 10 is a western blot illustrating the expression levels of exemplary modified proteins described herein in mitochondrial vs cytosolic fractions of spf/ash mice treated with mRNAs.

FIG. 11 is a plot illustrating expression levels of modified protein described herein in male C57BL/6 mice dosed with mRNAs (2262) having different modifications.

FIG. 12 is a western blot illustrating the protein expression levels in mitochondrial vs cytosolic fractions of spf/ash mice treated with mRNAs encoding an exemplary.

### DETAILED DESCRIPTION

### Definitions

The term “mitochondrial protein” is a protein identified as being localized within the mitochondria.

As used herein, the term “nuclear expressed mitochondrial protein” is a protein identified as being transcribed from nuclear DNA. A database with a listing of these proteins is the MitoCarta as described in the following: Sarah E. Calvo, Karl R. Clauser, Vamsi K. Mootha; MitoCarta2.0: an updated inventory of mammalian mitochondrial proteins, Nucleic Acids Research, Volume 44, Issue D1, 4 Jan. 2016, Pages D1251-D1257.

The term “ornithine transcarbamylase” as used interchangeably herein with “OTC” or “hOTC”, or “OTC HUMAN” generally refers to the human protein associated with UniPRotKB-P00480. The amino acid sequence for the wild type human OTC protein is represented herein by SEQ ID NO: 3.

As used herein, the term “therapeutic protein” means a protein that can be used to replace a protein in a patient deficient of such protein or wherein such protein does not possess the activity needed for normal function in a patient.

The term “nucleic acid,” in its broadest sense, includes any compound and/or substance that comprise a polymer of nucleotides. These polymers are often referred to as polynucleotides. Exemplary nucleic acids or polynucleotides described herein include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

As used herein, the term “polynucleotide” is generally used to refer to a nucleic acid (e.g., DNA or RNA). When RNA, such as mRNA, is specifically being referred to, the term polyribonucleotide may be used. The terms polynucleotide, polyribonucleotide, nucleic acid, ribonucleic acid, DNA, RNA, mRNA, and the like include such molecules that may be comprised of standard or unmodified residues; nonstandard or modified residues (e.g., analogs); and mixtures of standard and nonstandard (e.g., analogs) residues. In certain embodiments a polynucleotide or a polyribonucleotide is a modified polynucleotide or a polyribonucleotide. In the context of the present disclosure, for each RNA (polyribonucleotide) sequence listed herein, the corresponding DNA (polydeoxyribonucleotide or polynucleotide) sequence is contemplated and vice versa. “Polynucleotide” may be used interchangeably with the “oligomer”. Polynucleotide sequences shown herein are from left to right, 5′ to 3′, unless stated otherwise.

As used herein, the term “messenger RNA” (mRNA) refers to any polynucleotide which encodes a protein or polypeptide of interest and which is capable of being translated to produce the encoded protein or polypeptide of interest in vitro, in vivo, in situ or ex vivo.

As used herein, the term “translation” is the process in which ribosomes create polypeptides. In translation, messenger RNA (mRNA) is decoded by transfer RNAS (tRNAs) in a ribosome complex to produce a specific amino acid chain, or polypeptide. The coding region of a polynucleotide sequence (DNA or RNA), also known as the coding sequence or CDS, is capable of being converted to a protein or a fragment thereof by the process of translation.

As used herein, the term “codon-optimized” means a natural (or purposefully designed variant of a natural) coding sequence which has been redesigned by choosing different codons without altering the encoded protein amino acid sequence. Codon optimized sequence can increase the protein expression levels (Gustafsson et al., Codon bias and heterologous protein expression. 2004, Trends Biotechnol 22: 346-53) of the encoded proteins amongst providing other advantages. Variables such as high codon adaptation index (CAI), LowU method, mRNA secondary structures, cis-regulatory sequences, GC content and many other similar variables have been shown to somewhat correlate with protein expression levels (Villalobos et al., Gene Designer: a synthetic biology tool for constructing artificial DNA segments. 2006, BMC Bioinformatics 7:285). High CAI (codon adaptation index) method picks a most frequently used synonymous codon for an entire protein coding sequence. The most frequently used codon for each amino acid is deduced from 74,218 protein-coding genes from a human genome. The Low U method targets only U-containing codons that can be replaced with a synonymous codon with fewer U moieties. If there are a few choices for the replacement, the more frequently used codon will be selected. The remaining codons in the sequence are not changed by the Low U method. This method may be used in conjunction with the disclosed mRNAs to design coding sequences that are to be synthesized with, for example, 5-methoxyuridine or N1-methylpseudouridine.

As used herein, “modified” refers to a change in the state or structure of a molecule disclosed herein. The molecule may be changed in many ways including chemically, structurally or functionally. Preferably a polynucleotide or polypeptide of the disclosure are modified as compared to the native form of the polynucleotide or polypeptide or as compared to a reference polypeptide sequence or polynucleotide sequence. For example, mRNA disclosed herein may be modified by codon optimization, or by the insertion of non-natural nucleosides or nucleotides. Polypeptides may be modified, for example, by site specific amino acid deletions or substitutions to alter the properties of the polypeptide.

As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the present disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the present disclosure, two protein sequences are considered to be homologous if the proteins are at least about 50%, 60%, 70%, 80%, or 90% identical for at least one stretch of at least about 20 amino acids.

As used herein, the term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, can be performed by aligning the two sequences for optimal comparison purposes. In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984), BLASTP, BLASTN, and FASTA Altschul, S. F. et al., J. Molec. Biol., 215, 403 (1990).

An “effective amount” of the mRNA sequence encoding an open reading frame (ORF) protein or a corresponding composition thereof is generally that amount of mRNA that provides efficient ORF protein production in a cell. Preferably protein production using an mRNA composition described herein is more efficient than a composition containing a corresponding wild type mRNA encoding an ORF protein. Increased efficiency may be demonstrated by increased cell transfection (i.e., the percentage of cells transfected with the nucleic acid), increased protein translation from the nucleic acid, decreased nucleic acid degradation (as demonstrated, e.g., by increased duration of protein translation from a modified nucleic acid), or reduced innate immune response of the host cell. When referring to an ORF protein described herein, an effective amount is that amount of ORF protein that overcomes an ORF protein deficiency in a cell.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. Substantially isolated: By “substantially isolated” is meant that the compound is substantially separated from the environment in which it was formed or detected. Partial separation can include, for example, a composition enriched in the compound described herein. Substantial separation can include compositions containing at least about 50%, at least about 60%, at least about 70%, at least about 80%, at least about 90%, at least about 95%, at least about 97%, or at least about 99% by weight of the compound described herein, or salt thereof. Methods for isolating compounds and their salts are routine in the art.

As used herein, the term “subject” or “patient” refers to any organism to which a composition in accordance with the present disclosure may be administered, e.g., for experimental, diagnostic, prophylactic, and/or therapeutic purposes. Typical subjects include animals (e.g., mammals such as mice, rats, rabbits, non-human primates, and humans) and/or plants. Preferably “patient” refers to a human subject who may seek or be in need of treatment, requires treatment, is receiving treatment, will receive treatment, or a subject who is under care by a trained professional for a particular disease or condition.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

As used herein, the term “therapeutically effective amount” means an amount of an agent to be delivered (e.g., nucleic acid, protein or peptide, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

As used herein, a “total daily dose” is an amount given or prescribed in a 24 hr period. It may be administered as a single unit dose.

As used herein, the term “treating” refers to partially or completely alleviating, ameliorating, improving, relieving, delaying onset of, inhibiting progression of, reducing severity of, and/or reducing incidence of one or more symptoms or features of a protein deficiency. Treatment may be administered to a subject who does not exhibit signs of said protein deficiency and/or to a subject who exhibits only early signs of the protein deficiency for the purpose of decreasing the risk of developing pathology associated with the disease, disorder, and/or condition.

As used herein, the terms “transfect” or “transfection” mean the intracellular introduction of a nucleic acid into a cell, or preferably into a target cell. The introduced nucleic acid may be stably or transiently maintained in the target cell. The term “transfection efficiency” refers to the relative amount of nucleic acid up-taken by the target cell which is subject to transfection. In practice, transfection efficiency is estimated by the amount of a reporter nucleic acid product expressed by the target cells following transfection. Preferred are compositions with high transfection efficacies and in particular those compositions that minimize adverse effects which are mediated by transfection of non-target cells and tissues.

As used herein, the term “target cell” refers to a cell or tissue to which a composition of the disclosure is to be directed or targeted. In some embodiments, the target cells are deficient in a protein or enzyme of interest. For example, where it is desired to deliver a nucleic acid to a hepatocyte, the hepatocyte represents the target cell. In some embodiments, the nucleic acids and compositions of the present disclosure transfect the target cells on a discriminatory basis (i.e., do not transfect non-target cells). The compositions and methods of the present disclosure may be prepared to preferentially target a variety of target cells, which include, but are not limited to, hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior hom motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes and tumor cells.

Following transfection of one or more target cells by the compositions and nucleic acids described herein, expression of the protein encoded by such nucleic acid may be preferably stimulated and the capability of such target cells to express the protein of interest is enhanced. For example, transfection of a target cell with a mRNA will allow expression of the modified protein product following translation of the nucleic acid. The nucleic acids of the compositions and/or methods provided herein preferably encode a product (e.g., a protein, enzyme, polypeptide, peptide, functional RNA, and/or antisense molecule), and preferably encode a product whose in vivo production is desired.

As used herein “an OTC protein enzymatic activity” refers to enzyme activity that catalyzes the reaction between carbamoyl phosphate and ornithine to form citrulline as part of the urea cycle in mammals.

As used herein, the term “about” or “approximately” as applied to one or more values of interest, refers to a value that is similar to a stated reference value. In certain embodiments, the term “approximately” or “about” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

### Polynucleotide Sequences

The present disclosure provides improved methods and compositions for the treatment of diseases related to protein deficiency, for example ornithine transcarbamylase (OTC) deficiency using, for example, mRNA therapy to express a modified protein of the present disclosure. The present disclosure provides methods of treating a protein deficiency, comprising administering to a subject in need of treatment a composition comprising an mRNA sequence described herein encoding a stabilized modified human protein or active fragments of such stabilized modified human protein at an effective dose and an administration interval such that at least one symptom or feature of the protein deficiency is reduced in intensity, severity, or frequency or has delayed onset. The present disclosure also provides modified proteins encoded by the mRNA sequences wherein the modified proteins have improved properties such as enhanced stability and resistance to protein degradation and increased half-life as compared to wild type human proteins.

Preferably, the administration of an mRNA composition described herein results in an increased therapeutic protein expression or activity of the subject as compared to a control level. Preferably, the control level is a baseline serum therapeutic protein expression or activity level in the subject prior to the treatment and/or the control level is indicative of the average serum protein expression or activity level in patients without treatment.

Preferably, the proteins encoded by the mRNA described herein are produced from a heterologous mRNA construct comprising an open reading frame (ORF) also referred to herein as a “coding sequence” (CDS) encoding for a therapeutic protein. Preferably, the coding sequence is codon-optimized. Preferably, coding sequence is optimized to have a theoretical minimum of uridines possible to encode for a therapeutic protein. Preferably, the mRNA constructs described herein comprise one or more of the following features: a 5′ cap; a 5′UTR, a 5′UTR enhancer sequence, a Kozak sequence or a partial Kozak sequence, a 3′UTR, an open reading frame encoding a therapeutic protein protein and a poly A tail. Preferably, the mRNA constructs described herein can provide high-efficiency expression of a modified protein. The expression can be in vitro, ex vivo, or in vivo.

An exemplary human modified protein encoded by an mRNA described herein comprises a modified human OTC protein of SEQ ID NO: 4 shown in Table 1. SEQ ID NO: 4 has been modified from wild-type OTC of SEQ ID NO: 3 (Table 1) to remove one or more predicted ubiquitination sites resulting in a protein that is less susceptible to ubiquitination and degradation by ubiquitin ligases. The removal of predicted ubiquitination sites preferably comprises replacing N-terminus residues that have been found to support ubiquitination such as asparagine, arginine, leucine, lysine or phenylalanine with residues that have been found to be stabilizing against ubiquitination such as alanine, glycine, methionine, serine, threonine, valine and proline. Stabilization of the modified OTC protein of SEQ ID NO: 4 in this manner is particularly advantageous for preserving the stability of the modified OTC protein during its transport from the cytosol to the mitochondria wherein it exerts its enzymatic activity.

Preferably, a modified protein encoded by an mRNA described herein comprises a protein sequence that is at least 70%, 75%, 80%, 85%, 90%, 95%, 96%, 97%, 98%, 99%, or 100% identical to a corresponding human wild type protein, while retaining protein's biological and chemical activity such as catalytic activity.

TABLE 1
Selected OTC Nucleotide and Peptide Sequences

| | |
|---|---|
| mRNA coding | AUGCUGUUUAAUCUGAGGAUCCUGUUAAACAAUGCAGCUUUUAGAAA |
| sequence for wild | UGGUCACAACUUCAUGGUUCGAAAUUUUCGGUGUGGACAACCACUAC |
| type human OTC | AAAAUAAAGUGCAGCUGAAGGGCCGUGACCUUCUCACUCUAAAAAAC |
| | UUUACCGGAGAAGAAAUUAAAUAUAUGCUAUGGCUAUCAGCAGAUCU |
| | GAAAUUUAGGAUAAAACAGAAAGGAGAGUAUUUGCCUUUAUUGCAAG |
| | GGAAGUCCUUAGGCAUGAUUUUUGAGAAAAGAAGUACUCGAACAAGA |
| | UUGUCUACAGAAACAGGCUUUGCACUUCUGGGAGGACAUCCUUGUUU |
| | UCUUACCACACAAGAUAUUCAUUUGGGUGUGAAUGAAAGUCUCACGG |
| | ACACGGCCCGUGUAUUGUCUAGCAUGGCAGAUGCAGUAUUGGCUCGA |
| | GUGUAUAAACAAUCAGAUUUGGACACCCUGGCUAAAGAAGCAUCCAU |
| | CCCAAUUAUCAAUGGGCUGUCAGAUUUGUACCAUCCUAUCCAGAUCCU |
| | GGCUGAUUACCUCACGCUCCAGGAACACUAUAGCUCUCUGAAAGGUCU |
| | UACCCUCAGCUGGAUCGGGGAUGGGAACAAUAUCCUGCACUCCAUCAU |
| | GAUGAGCGCAGCGAAAUUCGGAAUGCACCUUCAGGCAGCUACUCCAAA |
| | GGGUUAUGAGCCGGAUGCUAGUGUAACCAAGUUGGCAGAGCAGUAUG |
| | CCAAAGAGAAUGGUACCAAGCUGUUGCUGACAAAUGAUCCAUUGGAA |
| | GCAGCGCAUGGAGGCAAUGUAUUAAUUACAGACACUUGGAUAAGCAU |
| | GGGACAAGAAGAGGAGAAGAAAAAGCGGCUCCAGGCUUUCCAAGGUU |
| | ACCAGGUUACAAUGAAGACUGCUAAAGUUGCUGCCUCUGACUGGACA |
| | UUUUUACACUGCUUGCCCAGAAAGCCAGAAGAAGUGGAUGAUGAAGU |
| | CUUUUAUUCUCCUCGAUCACUAGUGUUCCCAGAGGCAGAAAACAGAA |
| | AGUGGACAAUCAUGGCUGUCAUGGUGUCCCUGCUGACAGAUUACUCA |
| | CCUCAGCUCCAGAAGCCUAAAUUUUGA (SEQ ID NO: 1) |
| DNA coding | ATGCTGTTTAATCTGAGGATCCTGTTAAACAATGCAGCTTTTAGAAATGG |
| sequence for wild | TCACAACTTCATGGTTCGAAATTTTCGGTGTGGACAACCACTACAAAATA |
| type human OTC | AAGTGCAGCTGAAGGGCCGTGACCTTCTCACTCTAAAAAACTTTACCGG |
| | AGAAGAAATTAAATATATGCTATGGCTATCAGCAGATCTGAAATTTAGG |
| | ATAAAACAGAAAGGAGAGTATTTGCCTTTATTGCAAGGGAAGTCCTTAG |
| | GCATGATTTTTGAGAAAAGAAGTACTCGAACAAGATTGTCTACAGAAAC |
| | AGGCTTTGCACTTCTGGGAGGACATCCTTGTTTTCTTACCACACAAGATA |
| | TTCATTTGGGTGTGAATGAAAGTCTCACGGACACGGCCCGTGTATTGTCT |
| | AGCATGGCAGATGCAGTATTGGCTCGAGTGTATAAACAATCAGATTTGG |
| | ACACCCTGGCTAAAGAAGCATCCATCCCAATTATCAATGGGCTGTCAGA |
| | TTTGTACCATCCTATCCAGATCCTGGCTGATTACCTCACGCTCCAGGAAC |
| | ACTATAGCTCTCTGAAAGGTCTTACCCTCAGCTGGATCGGGGATGGGAA |
| | CAATATCCTGCACTCCATCATGATGAGCGCAGCGAAATTCGGAATGCAC |
| | CTTCAGGCAGCTACTCCAAAGGGTTATGAGCCGGATGCTAGTGTAACCA |
| | AGTTGGCAGAGCAGTATGCCAAAGAGAATGGTACCAAGCTGTTGCTGAC |
| | AAATGATCCATTGGAAGCAGCGCATGGAGGCAATGTATTAATTACAGAC |
| | ACTTGGATAAGCATGGGACAAGAAGAGGAGAAGAAAAAGCGGCTCCAG |
| | GCTTTCCAAGGTTACCAGGTTACAATGAAGACTGCTAAAGTTGCTGCCTC |
| | TGACTGGACATTTTTACACTGCTTGCCCAGAAAGCCAGAAGAAGTGGAT |
| | GATGAAGTCTTTTATTCTCCTCGATCACTAGTGTTCCCAGAGGCAGAAAA |
| | CAGAAAGTGGACAATCATGGCTGTCATGGTGTCCCTGCTGACAGATTAC |
| | TCACCTCAGCTCCAGAAGCCTAAATTTTGA (SEQ ID NO: 2) |
| Human wild type | MLFNLRILLNNAAFRNGHNFMVRNFRCGQPLQNKVQLKGRDLLTLKNFTG |
| OTC amino acid | EEIKYMLWLSADLKFRIKQKGEYLPLLQGKSLGMIFEKRSTRTRLSTETGFA |
| sequence | LLGGHPCFLTTQDIHLGVNESLTDTARVLSSMADAVLARVYKQSDLDTLAK |
| (The signal peptide | EASIPIFGLSDLYHPIQILADYLTLQEHYSSLKGLTLSWIGDGNNILHSIMMS |
| for mitochondrial | AAKFGMHLQAATPKGYEPDASVTKLAEQYAKENGTKLLLTNDPLEAAHGG |
| import is | NVLITDTWISMGQEEEKKKRLQAFQGYQVTMKTAKVAASDWTFLHCLPRK |
| underlined*) | PEEVDDEVFYSPRSLVFPEAENRKWTIMAVMVSLLTDYSPQLQKPKF |
| | (SEQ ID NO: 3) |
| Modified OTC amino | MLVFNLRILLNNAAFRNGHNFMVRNFRCGQPLQNRVQLKGRDLLTLKNFTGE |
| acid sequence | EIRYMLWLSADLKFRIKQKGEYLPLLQGKSLGMIFEKRSTRTRLSTETGFALLGGH |
| (The signal peptide | PCFLTTQDIHLGVNESLTDTARVLSSMADAVLARVYKQSDLDTLAKEASIPIINGLS |
| for mitochondrial | DLYHPIQILADYLTLQEHYSSLKGLTLSWIGDGNNILHSIMMSAAKFGMHLQAAT |
| import is | PKGYEPDASVTKLAEQYAKENGTKLLLTNDPLEAAHGGNVLITDTWISMGQEEEK |
| underlined*) | KKRLQAFQGYQVTMKTAKVAASDWTFLHCLPRKPEEVDDEVFYSPRSLVFPEAEN |
| | RKWTIMAVMVSLLTDYSPQLQKPKF (SEQ ID NO: 4) |

*The OTC protein comprises a signal peptide which is translated and which is responsible for translocation to the mitochondria. This signal peptide is represented by the first 32 amino acids as underlined in SEQ ID NO: 3 and SEQ ID NO: 4. The signal sequence of SEQ ID NO: 4 has also been modified as compared to SEQ ID NO: 3. An amino acid, valine is inserted at position 3 of SEQ ID NO: 4. This modification provides better mitochondrial localization of the modified OTC of SEQ ID NO: 4 as compared to wild type human OTC of SEQ ID NO: 3.

Preferably, the open reading frame (ORF) or coding sequence (CDS) of an mRNA sequence described herein encodes an amino acid sequence that is substantially identical to that of the corresponding human wild-type protein.

Preferably an mRNA described herein further comprises a sequence immediately downstream (i.e., in the 3′ direction from) of the CDS that creates a triple stop codon. The triple stop codon may be incorporated to enhance the efficiency of translation. In some embodiments, the translatable oligomer may comprise the sequence AUAAGUGAA (SEQ ID NO: 25) immediately downstream of a CDS of an mRNA sequence described herein.

Preferably, an mRNA described herein further comprises a 5′ untranslated region (UTR) sequence. As is understood in the art, the 5′ and/or 3′ UTR may affect an mRNA's stability or efficiency of translation. The 5′ UTR may be derived from an mRNA molecule known in the art to be relatively stable (e.g., histone, tubulin, globin, glyceraldehyde 1-phosphate dehydrogenase (GAPDH), actin, or citric acid cycle enzymes) to increase the stability of the translatable oligomer. In other embodiments, a 5′ UTR sequence may include a partial sequence of a cytomegalovirus (CMV) immediate-early 1 (IE1) gene.

Preferably, the 5′ UTR comprises a sequence selected from the 5′ UTRs of human IL-6, alanine aminotransferase 1, human apolipoprotein E, human fibrinogen alpha chain, human transthyretin, human haptoglobin, human alpha-1-antichymotrypsin, human antithrombin, human alpha-1-antitrypsin, human albumin, human beta globin, human complement C3, human complement C5, SynK (thylakoid potassium channel protein derived from the cyanobacteria, Synechocystis sp.), mouse beta globin, mouse albumin, and a tobacco etch virus, or fragments of any of the foregoing. Preferably, the 5′ UTR is derived from a tobacco etch virus (TEV). Preferably, an mRNA described herein comprises a 5′ UTR sequence that is derived from a gene expressed by Arabidopsis thaliana. Preferably, the 5′ UTR sequence of a gene expressed by Arabidopsis thaliana is AT1G58420. Preferred 5′ UTR sequences comprise SEQ ID NOS: 5-10, 125-127 and 227-247: as shown in Table 2.

TABLE 2
5′ UTR sequences

| Name | Sequence | Seq ID No.: |
|---|---|---|
| EV | UCAACACAACAUAUACAAAACAAACGAAUCUCAAGCAAUCAAGCA | SEQ ID NO: 5 |
| | UUCUACUUCUAUUGCAGCAAUUUAAAUCAUUUCUUUUAAAGCA | |
| | AAAGCAAUUUUCUGAAAAUUUUCACCAUUUACGAACGAUAG | |
| AT1G58420 | AUUAUUACAUCAAAACAAAAAGCCGCCA | SEQ ID NO: 6 |
| ARC5-2 | CUUAAGGGGGCGCUGCCUACGGAGGUGGCAGCCAUCUCCUUCU | SEQ ID NO: 7 |
| | CGGCAUCAAGCUUACCAUGGUGCCCCAGGCCCUGCUCUUGGUCC | |
| | CGCUGCUGGUGUUCCCCCUCUGCUUCGGCAAGUUCCCCAUCUAC | |
| | ACCAUCCCCGACAAGCUGGGGCCGUGGAGCCCCAUCGACAUCCA | |
| | CCACCUGUCCUGCCCCAACAACCUCGUGGUCGAGGACGAGGGCU | |
| | GCACCAACCUGAGCGGGUUCUCCUAC | |
| HCV | UGAGUGUCGU ACAGCCUCCA GGCCCCCCCC UCCCGGGAGA | SEQ ID NO: 8 |
| | GCCAUAGUGG UCUGCGGAACCGGUGAGUAC ACCGGAAUUG | |
| | CCGGGAAGAC UGGGUCCUUU CUUGGAUAAA | |
| | CCCACUCUAUGCCCGGCCAU UUGGGCGUGC CCCCGCAAGA | |
| | CUGCUAGCCG AGUAGUGUUG GGUUGCG | |
| HUMAN | AAUUAUUGGUUAAAGAAGUAUAUUAGUGCUAAUUUCCCUCCG | SEQ ID NO: 9 |
| ALBUMIN | UUUGUCCUAGCUUUUCUCUUCUGUCAACCCCACACGCCUUUGG | |
| | CACA | |
| EMCV | CUCCCUCCCC CCCCCCUAAC GUUACUGGCC GAAGCCGCUU | SEQ ID NO: 10 |
| | GGAAUAAGGC CGGUGUGCGU UUGUCUAUAU GUUAUUUUCC | |
| | ACCAUAUUGC CGUCUUUUGG CAAUGUGAGG GCCCGGAAAC | |
| | CUGGCCCUGU CUUCUUGACG AGCAUUCCUA GGGGUCUUUC | |
| | CCCUCUCGCC AAAGGAAUGC AAGGUCUGUU GAAUGUCGUG | |
| | AAGGAAGCAG UUCCUCUGGA AGCUUCUUGA AGACAAACAA | |
| | CGUCUGUAGC GACCCUUUGC AGGCAGCGGA ACCCCCCACC | |
| | UGGCGACAGG UGCCUCUGCG GCCAAAAGCC ACGUGUAUAA | |
| | GAUACACCUG CAAAGGCGGC ACAACCCCAG UGCCACGUUG | |
| | UGAGUUGGAU AGUUGUGGAA AGAGUCAAAU GGCUCUCCUC | |
| | AAGCGUAUUC AACAAGGGGC UGAAGGAUGC CCAGAAGGUA | |
| | CCCCAUUGUA UGGGAUCUGA UCUGGGGCCU CGGUGCACAU | |
| | GCUUUACGUG UGUUUAGUCG AGGUUAAAAA ACGUCUAGGC | |
| | CCCCCGAACC ACGGGGACGU GGUUUUCCUU UGAAAAACAC | |
| | GAUGAUAAU | |
| AT1G67090 | CACAAAGAGUAAAGAAGAACA | SEQ ID NO: 125 |
| AT1G35720 | AACACUAAAAGUAGAAGAAAA | SEQ ID NO: 126 |
| AT5G45900 | CUCAGAAAGAUAAGAUCAGCC | SEQ ID NO: 127 |
| AT5G61250 | AACCAAUCGAAAGAAACCAAA | SEQ ID NO: 230 |
| AT5G46430 | CUCUAAUCACCAGGAGUAAAA | SEQ ID NO: 231 |
| AT5G47110 | GAGAGAGAUCUUAACAAAAAA | SEQ ID NO: 232 |
| AT1G03110 | UGUGUAACAACAACAACAACA | SEQ ID NO: 233 |
| AT3G12380 | CCGCAGUAGGAAGAGAAAGCC | SEQ ID NO: 234 |
| AT5G45910 | AAAAAAAAAAGAAAUCAUAAA | SEQ ID NO: 235 |
| AT1G07260 | GAGAGAAGAAAGAAGAAGACG | SEQ ID NO: 236 |
| AT3G55500 | CAAUUAAAAAUACUUACCAAA | SEQ ID NO: 237 |
| AT3G46230 | GCAAACAGAGUAAGCGAAACG | SEQ ID NO: 238 |
| AT2G36170 | GCGAAGAAGACGAACGCAAAG | SEQ ID NO: 239 |
| AT1G10660 | UUAGGACUGUAUUGACUGGCC | SEQ ID NO: 240 |
| AT4G14340 | AUCAUCGGAAUUCGGAAAAAG | SEQ ID NO: 241 |
| AT1G49310 | AAAACAAAAGUUAAAGCAGAC | SEQ ID NO: 242 |
| AT4G14360 | UUUAUCUCAAAUAAGAAGGCA | SEQ ID NO: 243 |
| AT1G28520 | GGUGGGGAGGUGAGAUUUCUU | SEQ ID NO: 244 |
| AT1G20160 | UGAUUAGGAAACUACAAAGCC | SEQ ID NO: 245 |
| AT5G37370 | CAUUUUUCAAUUUCAUAAAAC | SEQ ID NO: 246 |
| AT4G11320 | UUACUUUUAAGCCCAACAAAA | SEQ ID NO: 247 |
| AT5G40850 | GGCGUGUGUGUGUGUUGUUGA | SEQ ID NO: 248 |
| AT1G06150 | GUGGUGAAGGGGAAGGUUUAG | SEQ ID NO: 249 |
| AT2G26080 | UUGUUUUUUUUUGGUUUGGUU | SEQ ID NO: 250 |

Preferably the 5′UTR sequence comprises SEQ ID NO: 6 (AT1G58420).

Preferably, an mRNA described herein comprises a translation enhancer sequence. Translation enhancer sequences enhance the translation efficiency of a mRNA described herein and thereby provide increased production of the protein encoded by the mRNA. The translation enhancer region may be located in the 5′ or 3′ UTR of an mRNA sequence. Examples of translation enhancer regions include naturally-occurring enhancer regions from TEV 5′UTR and Xenopus beta-globin 3′UTR. Preferred 5′ UTR enhancer sequences include but are not limited to those derived from mRNAs encoding human heat shock proteins (HSP) including HSP70-P2, HSP70-M1 HSP72-M2, HSP17.9 and HSP70-P1. Preferred translation enhancer sequences used in accordance with the embodiments of the present disclosure are represented by SEQ ID NOS: 11-15 as shown in Table 3.

TABLE 3
5′ UTR enhancers

| Name | Sequence | Seq ID No.: |
|---|---|---|
| HSP70-P2 | GUCAGCUUUCAAACUCUUUG | SEQ ID NO: 11 |
| | UUUCUUGUUUGUUGAUUGAG | |
| | AAUA | |
| HSP70-M1 | CUCUCGCCUGAGAAAAAAAA | SEQ ID NO: 12 |
| | UCCACGAACCAAUUUCUCAG | |
| | CAACCAGCAGCACG | |
| HSP72-M2 | ACCUGUGAGGGUUCGAAGGA | SEQ ID NO: 13 |
| | AGUAGCAGUGUUUUUUGUUC | |
| | CUAGAGGAAGAG | |
| HSP17.9 | ACACAGAAACAUUCGCAAAA | SEQ ID NO: 14 |
| | ACAAAAUCCCAGUAUCAAAA | |
| | UUCUUCUCUUUUUUUCAUAU | |
| | UUCGCAAAGAC | |
| HSP70-P1 | CAGAAAAAUUUGCUACAUUG | SEQ ID NO: 15 |
| | UUUCACAAACUUCAAAUAUU | |
| | AUUCAUUUAUUU | |

Preferably, an mRNA described herein comprises a Kozak sequence. As is understood in the art, a Kozak sequence is a short consensus sequence centered around the translational initiation site of eukaryotic mRNAs that allows for efficient initiation of translation of the mRNA. The ribosomal translation machinery recognizes the AUG initiation codon in the context of the Kozak sequence. A Kozak sequence, may be inserted upstream of the coding sequence for the therapeutic protein of interest, downstream of a 5′ UTR or inserted upstream of the coding sequence for the therapeutic protein of interest and downstream of a 5′ UTR. Preferably, an mRNA described herein comprises a Kozak sequence having the amino acid sequence GCCACC (SEQ ID NO: 23). Preferably an mRNA described herein comprises a partial Kozak sequence “p” having the amino acid sequence GCCA (SEQ ID NO: 24).

Preferably an mRNA described herein comprises a 3′UTR. Preferably, the 3′ UTR comprises a sequence selected from the 3′ UTRs of alanine aminotransferase 1, human apolipoprotein E, human fibrinogen alpha chain, human haptoglobin, human antithrombin, human alpha globin, human beta globin, human complement C3, human growth factor, human hepcidin, MALAT-1, mouse beta globin, mouse albumin, and Xenopus beta globin, or fragments of any of the foregoing. Preferably, the 3′ UTR is derived from Xenopus beta globin. Preferred 3′ UTR sequences include SEQ ID NOS 16-22 as shown in Table 4.

TABLE 4
3′ UTR sequences

| Name | Sequence | Seq ID No.: |
|---|---|---|
| XBG | CUAGUGACUGACUAGGAUCUGGUU | SEQ ID NO: 16 |
| | ACCACUAAACCAGCCUCAAGAACA | |
| | CCCGAAUGGAGUCUCUAAGCUACA | |
| | UAAUACCAACUUACACUUACAAAA | |
| | UGUUGUCCCCCAAAAUGUAGCCAU | |
| | UCGUAUCUGCUCCUAAUAAAAAGA | |
| | AAGUUUCUUCACAU | |
| HUMAN | UGCAAGGCUGGCCGGAAGCCCUUG | SEQ ID NO: 17 |
| HAPTOGLOBIN | CCUGAAAGCAAGAUUUCAGCCUGG | |
| | AAGAGGGCAAAGUGGACGGGAGUG | |
| | GACAGGAGUGGAUGCGAUAAGAUG | |
| | UGGUUUGAAGCUGAUGGGUGCCAG | |
| | CCCUGCAUUGCUGAGUCAAUCAAU | |
| | AAAGAGCUUUCUUUUGACCCAU | |
| HUMAN | ACGCCGAAGCCUGCAGCCAUGCGA | SEQ ID NO: 18 |
| APOLIPO- | CCCCACGCCACCCCGUGCCUCCUG | |
| PROTEIN E | CCUCCGCGCAGCCUGCAGCGGGAG | |
| | ACCCUGUCCCCGCCCCAGCCGUCC | |
| | UCCUGGGGUGGACCCUAGUUUAAU | |
| | AAAGAUUCACCAAGUUUCACGCA | |
| HCV | UAGAGCGGCAAACCCUAGCUACAC | SEQ ID NO: 19 |
| | UCCAUAGCUAGUUUCUUUUUUUUU | |
| | UGUUUUUUUUUUUUUUUUUUUUUU | |
| | UUUUUUUUUUUUUUUUUCCUUUCU | |
| | UUUCCUUCUUUUUUUCCUCUUUUC | |
| | UUGGUGGCUCCAUCUUAGCCCUAG | |
| | UCACGGCUAGCUGUGAAAGGUCCG | |
| | UGAGCCGCAUGACUGCAGAGAGUG | |
| | CCGUAACUGGUCUCUCUGCAGAUC | |
| | AUGU | |
| MOUSE | ACACAUCACAACCACAACCUUCUC | SEQ ID NO: 20 |
| ALBUMIN | AGGCUACCCUGAGAAAAAAAGACA | |
| | UGAAGACUCAGGACUCAUCUUUUC | |
| | UGUUGGUGUAAAAUCAACACCCUA | |
| | AGGAACACAAAUUUCUUUAAACAU | |
| | UUGACUUCUUGUCUCUGUGCUGCA | |
| | AUUAAUAAAAAAUGGAAAGAAUCU | |
| | AC | |
| HUMAN ALPHA | GCUGGAGCCUCGGUAGCCGUUCCU | SEQ ID NO: 21 |
| GLOBIN | CCUGCCCGCUGGGCCUCCCAACGG | |
| | GCCCUCCUCCCCUCCUUGCACCGG | |
| | CCCUUCCUGGUCUUUGAAUAAAGU | |
| | CUGAGUGGGCAGCA | |
| EMCV | UAGUGCAGUCAC UGGCACAACG | SEQ ID NO: 22 |
| | CGUUGCCCGG UAAGCCAAUC | |
| | GGGUAUACAC | |
| | GGUCGUCAUACUGCAGACAG | |
| | GGUUCUUCUA CUUUGCAAGA | |
| | UAGUCUAGAG UAGUAAAAUA | |
| | AAUAGUAUAAG | |

Preferably, an mRNA described herein comprises a 3′ tail region, which can serve to protect the mRNA from exonuclease degradation. The tail region may be a 3′poly(A) and/or 3′poly(C) region. Preferably, the tail region is a 3′ poly(A) tail. As used herein a “3′ poly(A) tail” is a polymer of sequential adenine nucleotides that can range in size from, for example: 10 to 250 sequential adenine nucleotides; 60-125 sequential adenine nucleotides, 90-125 sequential adenine nucleotides, 95-125 sequential adenine nucleotides, 95-121 sequential adenine nucleotides, 100 to 121 sequential adenine nucleotides, 110-121 sequential adenine nucleotides; sequential adenine nucleotides, 112-121 sequential adenine nucleotides; 114-121 adenine sequential nucleotides; and 115 to 121 sequential adenine nucleotides. Preferably a 3′ poly A tail as described herein comprise is 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125 sequential adenine nucleotides. 3′ Poly(A) tails can be added using a variety of methods known in the art, e.g., using poly(A) polymerase to add tails to synthetic or in vitro transcribed RNA. Other methods include the use of a transcription vector to encode poly A tails or the use of a ligase (e.g., via splint ligation using a T4 RNA ligase and/or T4 DNA ligase), wherein poly(A) may be ligated to the 3′ end of a sense RNA. Preferably, a combination of any of the above methods is utilized.

Preferably, an mRNA described herein comprises a 5′ cap. 5′-ends capped with various groups and their analogues are known in the art. The 5′ cap may be selected from m7GpppA, m7GpppC; unmethylated cap analogs (e.g., GpppG); dimethylated cap analog (e.g., m2,7GpppG), a trimethylated cap analog (e.g., m2,2,7GpppG), dimethylated symmetrical cap analogs (e.g., m7Gpppm7G), or anti reverse cap analogs (e.g., ARCA; m7, 2′OmeGpppG, m72′dGpppG, m7,3′OmeGpppG, m7,3′dGpppG and their tetraphosphate derivatives) (see, e.g., Jemielity, J. et al., RNA 9: 1108-1122 (2003). The 5′ cap may be an ARCA cap (3′-OMe-m7G(5′)pppG). The 5′ cap may be an mCAP (m7G(5′)ppp(5′)G, N7-Methyl-Guanosine-5′-Triphosphate-5′-Guanosine). The 5′ cap may be resistant to hydrolysis. A preferred 5′ cap is referred to herein as “m7GpppGm cap” also referred to herein as “Cap1” and has the following core structure:

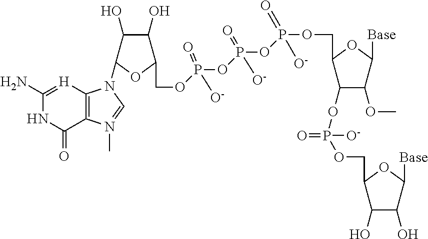

Preferably an mRNA described herein comprises one or more chemically modified nucleotides. Examples of nucleic acid monomers include non-natural, modified, and chemically-modified nucleotides, including any such nucleotides known in the art. mRNA sequences comprising chemically modified nucleotides have been shown to improve mRNA expression, expression rates, half-life and/or expressed protein concentrations. mRNA sequences comprising chemically modified nucleotides have also been useful to optimize protein localization thereby avoiding deleterious bio-responses such as the immune response and/or degradation pathways.

Examples of modified or chemically-modified nucleotides include 5-hydroxycytidines, 5-alkylcytidines, 5-hydroxyalkylcytidines, 5-carboxycytidines, 5-formylcytidines, 5-alkoxycytidines, 5-alkynylcytidines, 5-halocytidines, 2-thiocytidines, N⁴-alkylcytidines, N⁴-aminocytidines, N⁴-acetylcytidines, and N⁴,N⁴-dialkylcytidines.

Examples of modified or chemically-modified nucleotides include 5-hydroxycytidine, 5-methylcytidine, 5-hydroxymethylcytidine, 5-carboxycytidine, 5-formylcytidine, 5-methoxycytidine, 5-propynylcytidine, 5-bromocytidine, 5-iodocytidine, 2-thiocytidine; N4-methylcytidine, N4-aminocytidine, N4-acetylcytidine, and N4,N4-dimethylcytidine.

Examples of modified or chemically-modified nucleotides include 5-hydroxyuridines, 5-alkyluridines, 5-hydroxyalkyluridines, 5-carboxyuridines, 5-carboxyalkylesteruridines, 5-formyluridines, 5-alkoxyuridines, 5-alkynyluridines, 5-halouridines, 2-thiouridines, and 6-alkyluridines.

Examples of modified or chemically-modified nucleotides include 5-hydroxyuridine, 5-methyluridine, 5-hydroxymethyluridine, 5-carboxyuridine, 5-carboxymethylesteruridine, 5-formyluridine, 5-methoxyuridine (also referred to herein as “5MeOU”), 5-propynyluridine, 5-bromouridine, 5-fluorouridine, 5-iodouridine, 2-thiouridine, and 6-methyluridine.

Examples of modified or chemically-modified nucleotides include 5-methoxycarbonylmethyl-2-thiouridine, 5-methylaminomethyl-2-thiouridine, 5-carbamoylmethyluridine, 5-carbamoylmethyl-2′-O-methyluridine, 1-methyl-3-(3-amino-3-carboxypropy)pseudouridine, 5-methylaminomethyl-2-selenouridine, 5-carboxymethyluridine, 5-methyldihydrouridine, 5-taurinomethyluridine, 5-taurinomethyl-2-thiouridine, 5-(isopentenylaminomethyl)uridine, 2′-O-methylpseudouridine, 2-thio-2′O-methyluridine, and 3,2′-O-dimethyluridine.

Examples of modified or chemically-modified nucleotides include N6-methyladenosine, 2-aminoadenosine, 3-methyladenosine, 8-azaadenosine, 7-deazaadenosine, 8-oxoadenosine, 8-bromoadenosine, 2-methylthio-N6-methyladenosine, N6-isopentenyladenosine, 2-methylthio-N6-isopentenyladenosine, N6-(cis-hydroxyisopentenyl)adenosine, 2-methylthio-N6-(cis-hydroxyisopentenyl)adenosine, N6-glycinylcarbamoyladenosine, N6-threonylcarbamoyl-adenosine, N6-methyl-N6-threonylcarbamoyl-adenosine, 2-methylthio-N6-threonylcarbamoyl-adenosine, N6,N6-dimethyladenosine, N6-hydroxynorvalylcarbamoyladenosine, 2-methylthio-N6-hydroxynorvalylcarbamoyl-adenosine, N6-acetyl-adenosine, 7-methyl-adenine, 2-methylthio-adenine, 2-methoxy-adenine, alpha-thio-adenosine, 2′-O-methyl-adenosine, N6,2′-O-dimethyl-adenosine, N6,N6,2′-O-trimethyl-adenosine, 1,2′-O-dimethyl-adenosine, 2′-O-ribosyladenosine, 2-amino-N6-methyl-purine, 1-thio-adenosine, 2′-F-ara-adenosine, 2′-F-adenosine, 2′-OH-ara-adenosine, and N6-(19-amino-pentaoxanonadecyl)-adenosine.

Examples of modified or chemically-modified nucleotides include N1-alkylguanosines, N2-alkylguanosines, thienoguanosines, 7-deazaguanosines, 8-oxoguanosines, 8-bromoguanosines, 06-alkylguanosines, xanthosines, inosines, and N1-alkylinosines.

Examples of modified or chemically-modified nucleotides include N1-methylguanosine, N2-methylguanosine, thienoguanosine, 7-deazaguanosine, 8-oxoguanosine, 8-bromoguanosine, 06-methylguanosine, xanthosine, inosine, and N1-methylinosine.

Examples of modified or chemically-modified nucleotides include pseudouridines. Examples of pseudouridines include N1-alkylpseudouridines, N1-cycloalkylpseudouridines, N1-hydroxypseudouridines, N1-hydroxyalkylpseudouridines, N1-phenylpseudouridines, N1-phenylalkylpseudouridines, N1-aminoalkylpseudouridines, N3-alkylpseudouridines, N6-alkylpseudouridines, N6-alkoxypseudouridines, N6-hydroxypseudouridines, N6-hydroxyalkylpseudouridines, N6-morpholinopseudouridines, N6-phenylpseudouridines, and N6-halopseudouridines. Examples of pseudouridines include N1-alkyl-N6-alkylpseudouridines, N1-alkyl-N6-alkoxypseudouridines, N1-alkyl-N6-hydroxypseudouridines, N1-alkyl-N6-hydroxyalkylpseudouridines, N1-alkyl-N6-morpholinopseudouridines, N1-alkyl-N6-phenylpseudouridines, and N1-alkyl-N6-halopseudouridines. In these examples, the alkyl, cycloalkyl, and phenyl substituents may be unsubstituted, or further substituted with alkyl, halo, haloalkyl, amino, or nitro substituents.

Examples of pseudouridines include N1-methylpseudouridine (also referred to herein as “N1MPU”), N1-ethylpseudouridine, N1-propylpseudouridine, N1-cyclopropylpseudouridine, N1-phenylpseudouridine, N1-aminomethylpseudouridine, N3-methylpseudouridine, N1-hydroxypseudouridine, and N1-hydroxymethylpseudouridine.

Examples of nucleic acid monomers include modified and chemically-modified nucleotides, including any such nucleotides known in the art.

Examples of modified and chemically-modified nucleotide monomers include any such nucleotides known in the art, for example, 2′-O-methyl ribonucleotides, 2′-O-methyl purine nucleotides, 2′-deoxy-2′-fluoro ribonucleotides, 2′-deoxy-2′-fluoro pyrimidine nucleotides, 2′-deoxy ribonucleotides, 2′-deoxy purine nucleotides, universal base nucleotides, 5-C-methyl-nucleotides, and inverted deoxyabasic monomer residues.

Examples of modified and chemically-modified nucleotide monomers include 3′-end stabilized nucleotides, 3′-glyceryl nucleotides, 3′-inverted abasic nucleotides, and 3′-inverted thymidine.

Examples of modified and chemically-modified nucleotide monomers include locked nucleic acid nucleotides (LNA), 2′-0,4′-C-methylene-(D-ribofuranosyl) nucleotides, 2′-methoxyethoxy (MOE) nucleotides, 2′-methyl-thio-ethyl, 2′-deoxy-2′-fluoro nucleotides, and 2′-O-methyl nucleotides. In an exemplary embodiment, the modified monomer is a locked nucleic acid nucleotide (LNA).

Examples of modified and chemically-modified nucleotide monomers include 2′,4′-constrained 2′-O-methoxyethyl (cMOE) and 2′-O-Ethyl (cEt) modified DNAs.

Examples of modified and chemically-modified nucleotide monomers include 2′-amino nucleotides, 2′-O-amino nucleotides, 2′-C-allyl nucleotides, and 2′-O-allyl nucleotides.

Examples of modified and chemically-modified nucleotide monomers include N6-methyladenosine nucleotides.

Examples of modified and chemically-modified nucleotide monomers include nucleotide monomers with modified bases 5-(3-amino)propyluridine, 5-(2-mercapto)ethyluridine, 5-bromouridine; 8-bromoguanosine, or 7-deazaadenosine.

Examples of modified and chemically-modified nucleotide monomers include 2′-O-aminopropyl substituted nucleotides.

Examples of modified and chemically-modified nucleotide monomers include replacing the 2′-OH group of a nucleotide with a 2′-R, a 2′-OR, a 2′-halogen, a 2′-SR, or a 2′-amino, where R can be H, alkyl, alkenyl, or alkynyl.

Example of base modifications described above can be combined with additional modifications of nucleoside or nucleotide structure, including sugar modifications and linkage modifications. Certain modified or chemically-modified nucleotide monomers may be found in nature.

Preferred nucleotide modifications include N¹-methylpseudouridine and 5-methoxyuridine. The constructs for preferred mRNA sequences are provided in Table 5.

TABLE 5
Exemplary mRNA Constructs

| mRNA | | | | | | | mRNA |
|---|---|---|---|---|---|---|---|
| Construct | | | | Therapeutic | | 3′ Poly | Construct |
| No. | Cap | 5′UTR | Kozak* | Protein ORF | 3′UTR | A Tail | SEQ ID NO: |
| 563 | Cap1 | TEV | Yes | ORF | XBG | Yes | 26 |
| | | | | Sequence | | | |
| 564 | Cap1 | TEV | Yes | ORF | XBG | Yes | 27 |
| | | | | Sequence | | | |
| 565 | Cap1 | TEV | Yes | ORF | XBG | Yes | 28 |
| | | | | Sequence | | | |
| 566 | Cap1 | TEV | Yes | SEQ ID NO: 3 | XBG | Yes | 29 |
| 567 | Cap1 | TEV | Yes | SEQ ID NO: 3 | XBG | Yes | 30 |
| 568 | Cap1 | TEV | Yes | SEQ ID NO: 3 | XBG | Yes | 31 |
| 569 | Cap1 | TEV | Yes | SEQ ID NO: 3 | XBG | Yes | 32 |
| 570 | Cap1 | TEV | Yes | ORF | XBG | Yes | 33 |
| | | | | SEQUENCE | | | |
| 571 | Cap1 | TEV | Yes | ORF | XBG | Yes | 34 |
| | | | | SEQUENCE | | | |
| 572 | Cap1 | TEV | Yes | ORF | XBG | Yes | 35 |
| | | | | SEQUENCE | | | |
| 573 | Cap1 | TEV | Yes | ORF | XBG | Yes | 36 |
| | | | | SEQUENCE | | | |
| 574 | Cap1 | TEV | Yes | ORF | XBG | Yes | 37 |
| | | | | SEQUENCE | | | |
| 575 | Cap1 | TEV | P | ORF | XBG | Yes | 38 |
| | | | | SEQUENCE | | | |
| 708 | Cap1 | TEV | P | ORF | XBG | Yes | 39 |
| | | | | SEQUENCE | | | |
| 709 | Cap1 | TEV | P | ORF | XBG | Yes | 40 |
| | | | | SEQUENCE | | | |
| 710 | Cap1 | TEV | P | ORF | XBG | Yes | 41 |
| | | | | SEQUENCE | | | |
| 711 | Cap1 | TEV | P | ORF | XBG | Yes | 42 |
| | | | | SEQUENCE | | | |
| 712 | Cap1 | TEV | Yes | ORF | XBG | Yes | 43 |
| | | | | SEQUENCE | | | |
| 713 | Cap1 | TEV | Yes | ORF | XBG | Yes | 44 |
| | | | | SEQUENCE | | | |
| 714 | Cap1 | TEV | Yes | ORF | XBG | Yes | 45 |
| | | | | SEQUENCE | | | |
| 715 | Cap1 | TEV | Yes | ORF | XBG | Yes | 46 |
| | | | | SEQUENCE | | | |
| 716 | Cap1 | TEV | Yes | ORF | XBG | Yes | 47 |
| | | | | SEQUENCE | | | |
| 717 | Cap1 | TEV | Yes | ORF | XBG | Yes | 48 |
| | | | | SEQUENCE | | | |
| 718 | Cap1 | TEV | Yes | ORF | XBG | Yes | 49 |
| | | | | SEQUENCE | | | |
| 719 | Cap1 | TEV | Yes | ORF | XBG | Yes | 50 |
| | | | | SEQUENCE | | | |
| 720 | Cap1 | TEV | Yes | ORF | XBG | Yes | 51 |
| | | | | SEQUENCE | | | |
| 721 | Cap1 | TEV | Yes | ORF | XBG | Yes | 52 |
| | | | | SEQUENCE | | | |
| 722 | Cap1 | TEV | Yes | ORF | XBG | Yes | 53 |
| | | | | SEQUENCE | | | |
| 723 | Cap1 | TEV | Yes | ORF | XBG | Yes | 54 |
| | | | | SEQUENCE | | | |
| 724 | Cap1 | TEV | Yes | ORF | XBG | Yes | 55 |
| | | | | SEQUENCE | | | |
| 725 | Cap1 | TEV | Yes | ORF | XBG | Yes | 56 |
| | | | | SEQUENCE | | | |
| 726 | Cap1 | TEV | Yes | ORF | XBG | Yes | 57 |
| | | | | SEQUENCE | | | |
| 727 | Cap1 | TEV | Yes | ORF | XBG | Yes | 58 |
| | | | | SEQUENCE | | | |
| 728 | Cap1 | TEV | Yes | ORF | XBG | Yes | 59 |
| | | | | SEQUENCE | | | |
| 729 | Cap1 | TEV | Yes | ORF | XBG | Yes | 60 |
| | | | | SEQUENCE | | | |
| 1787 | Cap1 | ARC5-2 | No | ORF | Hu haptoglob | Yes | 61 |
| | | | | SEQUENCE | | | |
| 1788 | Cap1 | AT1G58420 | P | ORF | Hu ApoE | Yes | 62 |
| | | | | SEQUENCE | | | |
| 1789 | Cap1 | ARC5-2 | No | ORF | Hu haptoglob | Yes | 63 |
| | | | | SEQUENCE | | | |
| 1790 | Cap1 | AT1G58420 | P | ORF | Hu ApoE | Yes | 64 |
| | | | | SEQUENCE | | | |
| 1791 | Cap1 | ARC5-2 | No | ORF | Hu haptoglob | Yes | 65 |
| | | | | SEQUENCE | | | |
| 1792 | Cap1 | HCV5′ | P | ORF | HCV3′ | Yes | 66 |
| | | | | SEQUENCE | | | |
| 1793 | Cap1 | AT1G58420 | P | ORF | Hu a-glob | Yes | 67 |
| | | | | SEQUENCE | | | |
| 1794 | Cap1 | AT1G58420 | P | ORF | Hu a-glob | Yes | 68 |
| | | | | SEQUENCE | | | |
| 1795 | Cap1 | AT1G58420 | P | ORF | Hu a-glob | Yes | 69 |
| | | | | SEQUENCE | | | |
| 1796 | Cap1 | Hu alb | No | ORF | Ms Alb | Yes | 70 |
| | | | | SEQUENCE | | | |
| 1797 | Cap1 | Hu alb | No | ORF | Ms Alb | Yes | 71 |
| | | | | SEQUENCE | | | |
| 1798 | Cap1 | Hu alb | No | ORF | Ms Alb | Yes | 72 |
| | | | | SEQUENCE | | | |
| 1799 | Cap1 | AT1G58420 | Yes | ORF | Hu a-glob | Yes | 73 |
| | | | | SEQUENCE | | | |
| 1800 | Cap1 | Hu alb | No | ORF | Ms Alb | Yes | 74 |
| | | | | SEQUENCE | | | |
| 1801 | Cap1 | AT1G58420 | P | ORF | Hu ApoE | Yes | 75 |
| | | | | SEQUENCE | | | |
| 1802 | Cap1 | ARC5-2 | No | ORF | Hu haptoglob | Yes | 76 |
| | | | | SEQUENCE | | | |
| 1803 | Cap1 | TEV | Yes | ORF | XBG | Yes | 77 |
| | | | | SEQUENCE | | | |
| 1804 | Cap1 | TEV | Yes | ORF | XBG | Yes | 78 |
| | | | | SEQUENCE | | | |
| 1805 | Cap1 | TEV | Yes | ORF | XBG | Yes | 79 |
| | | | | SEQUENCE | | | |
| 1806 | Cap1 | TEV | Yes | ORF | XBG | Yes | 80 |
| | | | | SEQUENCE | | | |
| 1808 | Cap1 | TEV | Yes | ORF | XBG | Yes | 81 |
| | | | | SEQUENCE | | | |
| 1809 | Cap1 | TEV | Yes | ORF | XBG | Yes | 82 |
| | | | | SEQUENCE | | | |
| 1816 | Cap1 | TEV | P | ORF | XBG | Yes | 83 |
| | | | | SEQUENCE | | | |
| 1822 | Cap1 | TEV | Yes | ORF | XBG | Yes | 84 |
| | | | | SEQUENCE | | | |
| 1823 | Cap1 | TEV | Yes | ORF | XBG | Yes | 85 |
| | | | | SEQUENCE | | | |
| 1840 | Cap1 | EMCV | No | ORF | EMCV | Yes | 86 |
| | | | | SEQUENCE | | | |
| 1841 | Cap1 | EMCV | No | ORF | EMCV | Yes | 87 |
| | | | | SEQUENCE | | | |
| 1842 | Cap1 | EMCV | No | ORF | EMCV | Yes | 88 |
| | | | | SEQUENCE | | | |
| 1843 | Cap1 | HSP70-P2- | Yes | ORF | XBG | Yes | 89 |
| | | TEV | | SEQUENCE | | | |
| 1844 | Cap1 | HSP70-M1- | Yes | ORF | XBG | Yes | 90 |
| | | TEV | | SEQUENCE | | | |
| 1845 | Cap1 | HSP70-M2- | Yes | ORF | XBG | Yes | 91 |
| | | TEV | | SEQUENCE | | | |
| 1846 | Cap1 | HSP17.9- | Yes | ORF | XBG | Yes | 92 |
| | | TEV | | SEQUENCE | | | |
| 1847 | Cap1 | HSP70-P1- | Yes | ORF | XBG | Yes | 93 |
| | | TEV | | SEQUENCE | | | |
| 1882 | Cap1 | TEV | Yes | ORF | XBG | Yes | 94 |
| | | | | SEQUENCE | | | |
| 1883 | Cap1 | TEV | Yes | ORF | XBG | Yes | 95 |
| | | | | SEQUENCE | | | |
| 1884 | Cap1 | TEV | Yes | ORF | XBG | Yes | 96 |
| | | | | SEQUENCE | | | |
| 1885 | Cap1 | TEV | Yes | ORF | XBG | Yes | 97 |
| | | | | SEQUENCE | | | |
| 1886 | Cap1 | TEV | Yes | ORF | XBG | Yes | 98 |
| | | | | SEQUENCE | | | |
| 1887 | Cap1 | TEV | Yes | ORF | XBG | Yes | 99 |
| | | | | SEQUENCE | | | |
| 1888 | Cap1 | TEV | Yes | ORF | XBG | Yes | 100 |
| | | | | SEQUENCE | | | |
| 1889 | Cap1 | TEV | Yes | ORF | XBG | Yes | 101 |
| | | | | SEQUENCE | | | |
| 1890 | Cap1 | TEV | Yes | ORF | XBG | Yes | 102 |
| | | | | SEQUENCE | | | |
| 1891 | Cap1 | TEV | Yes | ORF | XBG | Yes | 103 |
| | | | | SEQUENCE | | | |
| 1898 | Cap1 | TEV | Yes | ORF | XBG | Yes | 104 |
| | | | | SEQUENCE | | | |
| 1899 | Cap1 | TEV | Yes | ORF | XBG | Yes | 105 |
| | | | | SEQUENCE | | | |
| 1900 | Cap1 | TEV | Yes | ORF | XBG | Yes | 106 |
| | | | | SEQUENCE | | | |
| 1903 | Cap1 | TEV | P | ORF | XBG | Yes | 107 |
| | | | | SEQUENCE | | | |
| 1904 | Cap1 | TEV | P | ORF | XBG | Yes | 108 |
| | | | | SEQUENCE | | | |
| 1905 | Cap1 | TEV | P | ORF | XBG | Yes | 109 |
| | | | | SEQUENCE | | | |
| 1906 | Cap1 | TEV | P | ORF | XBG | Yes | 110 |
| | | | | SEQUENCE | | | |
| 1907 | Cap1 | TEV | P | ORF | XBG | Yes | 111 |
| | | | | SEQUENCE | | | |
| 1908 | Cap1 | TEV | P | ORF | XBG | Yes | 112 |
| | | | | SEQUENCE | | | |
| 1915 | Cap1 | HSP70-P1- | Yes | ORF | Hu a-glob | Yes | 113 |
| | | AT1G58420 | | SEQUENCE | | | |
| 1916 | Cap1 | HSP70-P1- | Yes | ORF | Hu a-glob | Yes | 114 |
| | | AT1G58420 | | SEQUENCE | | | |
| 1917 | Cap1 | HSP70-P1- | Yes | ORF | Hu a-glob | Yes | 115 |
| | | AT1G58420 | | SEQUENCE | | | |
| 1918 | Cap1 | HSP70-P1- | Yes | ORF | Hu a-glob | Yes | 116 |
| | | AT1G58420 | | SEQUENCE | | | |
| 1919 | Cap1 | AT1G58420 | Yes | ORF | Hu a-glob | Yes | 117 |
| | | | | SEQUENCE | | | |
| 1920 | Cap1 | AT1G58420 | Yes | ORF | Hu a-glob | Yes | 118 |
| | | | | SEQUENCE | | | |
| 1921 | Cap1 | AT1G58420 | Yes | SEQ ID NO: | Hu a-glob | Yes | 119 |
| | | | | 4** | | | |
| 1925 | Cap1 | TEV | Yes | ORF | XBG | Yes | 120 |
| | | | | SEQUENCE | | | |
| 1926 | Cap1 | AT1G58420 | Yes | ORF | Hu a-glob | Yes | 121 |
| | | | | SEQUENCE | | | |
| 1927 | Cap1 | AT1G58420 | Yes | ORF | Hu a-glob | Yes | 122 |
| | | | | SEQUENCE | | | |
| 1928 | Cap1 | AT1G58420 | Yes | ORF | Hu a-glob | Yes | 123 |
| | | | | SEQUENCE | | | |
| 1929 | Cap1 | HSP70-P1- | Yes | ORF | Hu a-glob | Yes | 124 |
| | | AT1G58420 | | SEQUENCE | | | |
| 2016 | Cap1 | AT1G58420 | Yes | ORF | Hu a-glob | Yes | 73 |
| | | | | SEQUENCE | | | |
| 2260 | Cap1 | AT1G58420 | Yes | SEQ ID NO: | Hu a-glob | Yes | 119 |
| | | | | 4** | | | |
| 2262 | Cap1 | AT1G58420 | Yes | SEQ IS NO: | Hu a-glob | Yes | 119 |
| | | | | 4** | | | |

*Kozak sequence defined as GCCACC (SEQ ID NO: 23). Partial (P) Kozak defined as GCCA (SEQ ID NO: 24).
**Construct encodes modified human OTC protein of SEQ ID NO: 4.

Preferred mRNA sequences include all of the mRNA sequences listed in Table 5. Preferred mRNA sequences include all of the mRNA sequences listed wherein, 0% to 100%, preferably 1% to 100%, preferably 25% to 100%, preferably 50% to 100% and preferably 75% to 100% of the uracil nucleotides of the mRNA sequences are modified. Preferably, 1% to 100% of the uracil nucleotides are N1-methylpseudouridine or 5-methoxyuridine. Preferably 100% of the uracil nucleotides are N1-methylpseudouridine. Preferably 100% of the uracil nucleotides are 5-methoxyuridine.

Preferred mRNA sequences comprise a 5′ cap, a 5′UTR that is derived from a gene expressed by Arabidopsis thaliana, an optional translation enhancer sequence, an optional Kozak sequence or partial Kozak sequence, a codon optimized coding sequence (CDS/ORF) coding for a human protein of interest, a 3′ UTR and a poly A tail. Preferably, the 5′ UTR that is derived from a gene expressed by Arabidopsis thaliana is selected from found in Table 5. Preferably, the 5′ UTR that is derived from a gene expressed by Arabidopsis thaliana is selected from the group consisting of: SEQ ID NO: 6, SEQ ID NOS: 125-127 and SEQ ID NOS: 227-247. Preferably the 5′ UTR sequence is AT1G58420 having the sequence of SEQ ID NO: 6. Preferably, the uracil content of the codon optimized sequence has been reduced with respect to the percentages of uracil content of SEQ ID NO: 1. Preferably, 0% to 100% of the uracil nucleotides of the mRNA sequences are modified. Preferably, 0% to 100% of the uracil nucleotides are N1-methylpseudouridine or 5-methoxyuridine. Preferably 100% of the uracil nucleotides are N1-methylpseudouridine. Preferably 100% of the uracil nucleotides are 5-methoxyuridine.

Preferred mRNA constructs comprise codon optimized coding sequences and a 5′ UTR from a gene expressed by Arabidopsis thaliana and are selected from: SEQ ID NOS: 62, 67, 68, 69, 73, 113-119, 121-127.

An exemplary mRNA sequences includes the mRNA construct having a codon optimized ORF encoding modified OTC of SEQ ID NO: 4.

Preferably 100% of the uridine nucleotides of mRNA constructs 1799 (SEQ ID NO:73) and 1921 (SEQ ID NO:119), are N1-methylpseudouridine. Preferably 100% of the uracil nucleotides of mRNA constructs 1799 (SEQ ID NO:73), and 1921 (SEQ ID NO:119), are 5-methoxyuridine.

Design and Synthesis of the mRNA Sequences

mRNA for use in accordance with the disclosure may be prepared according to any available technique including, but not limited to chemical synthesis, in vitro transcription (IVT) or enzymatic or chemical cleavage of a longer precursor, etc. Methods of synthesizing RNAs are known in the art.

In some embodiments, mRNA is produced from a primary complementary DNA (cDNA) construct. The process of design and synthesis of the primary cDNA constructs described herein generally includes the steps of gene construction, mRNA production (either with or without modifications) and purification. In the IVT method, a target polynucleotide sequence encoding the modified therapeutic protein of interest is first selected for incorporation into a vector which will be amplified to produce a cDNA template. Optionally, the target polynucleotide sequence and/or any flanking sequences may be codon optimized. The cDNA template is then used to produce mRNA through in vitro transcription (IVT). After production, the mRNA may undergo purification and clean-up processes. The steps of which are provided in more detail below.

The step of gene construction may include, but is not limited to gene synthesis, vector amplification, plasmid purification, plasmid linearization and clean-up, and cDNA template synthesis and clean-up. Once a modified protein (e.g. SEQ ID NO: 4) is selected for production, a primary construct is designed. Within the primary construct, a first region of linked nucleosides encoding the polypeptide of interest may be constructed using an open reading frame (ORF) of a selected nucleic acid (DNA or RNA) transcript. The ORF may comprise the wild type ORF, an isoform, variant or a fragment thereof. As used herein, an “open reading frame” or “ORF” is meant to refer to a nucleic acid sequence (DNA or RNA) which is capable of encoding a polypeptide of interest. ORFs often begin with the start codon, ATG and end with a nonsense or termination codon or signal.

Further, nucleotide sequence of any region of the mRNA or DNA template may be codon optimized. Codon optimization methods are known in the art and may be useful in efforts to achieve one or more of several goals. These goals include to match codon frequencies in target and host organisms to ensure proper folding, to bias GC content to increase mRNA stability or reduce secondary structures, to minimize tandem repeat codons or base runs that may impair gene construction or expression, to customize transcriptional and translational control regions, to insert or remove protein trafficking sequences, to remove/add post translation modification sites in encoded protein (e.g. glycosylation sites), to add, remove or shuffle protein domains, to insert or delete restriction sites, to modify ribosome binding sites and mRNA degradation sites, to adjust translational rates to allow the various domains of the protein to fold properly, or to reduce or eliminate problematic secondary structures within the mRNA. Suitable codon optimization tools, algorithms and services are known in the art.

Preferably, the primary cDNA template may include reducing the occurrence or frequency of appearance of certain nucleotides in the template strand. For example, the occurrence of a nucleotide in a template may be reduced to a level below 25% of nucleotides in the template. In further examples, the occurrence of a nucleotide in a template may be reduced to a level below 20% of nucleotides in the template. In some examples, the occurrence of a nucleotide in a template may be reduced to a level below 16% of nucleotides in the template. Preferably, the occurrence of a nucleotide in a template may be reduced to a level below 15%, and preferably may be reduced to a level below 12% of nucleotides in the template.

For example, the present disclosure provides nucleic acids wherein with altered uracil content at least one codon in the wild-type sequence has been replaced with an alternative codon to generate a uracil-altered sequence. Altered uracil sequences can have at least one of the following properties:

(i) an increase or decrease in global uracil content (i.e., the percentage of uracil of the total nucleotide content in the nucleic acid of a section of the nucleic acid, e.g., the open reading frame); or,

(ii) an increase or decrease in local uracil content (i.e., changes in uracil content are limited to specific subsequences); or,

(iii) a change in uracil distribution without a change in the global uracil content; or,

(iv) a change in uracil clustering (e.g., number of clusters, location of clusters, or distance between clusters); or,

(v) combinations thereof.

Preferably, the percentage of uracil nucleobases in the nucleic acid sequence is reduced with respect to the percentage of uracil nucleobases in the wild-type nucleic acid sequence. For example, 30% of nucleobases may be uracil in the wild-type sequence but the nucleobases that are uracil are preferably lower than 15%, preferably lower than 12% and preferably lower than 10% of the nucleobases in the in the nucleic acid sequences of the disclosure. The percentage uracil content can be determined by dividing the number of uracil in a sequence by the total number of nucleotides and multiplying by 100.

Preferably, the percentage of uracil nucleobases in a subsequence of the nucleic acid sequence is reduced with respect to the percentage of uracil nucleobases in the corresponding subsequence of the wild-type sequence. For example, the wild-type sequence may have a 5′-end region (e.g., 30 codons) with a local uracil content of 30%, and the uracil content in that same region could be reduced to preferably 15% or lower, preferably 12% or lower and preferably 10% or lower in the nucleic acid sequences of the disclosure.

Preferably, codons in the nucleic acid sequence of the invention reduce or modify, for example, the number, size, location, or distribution of uracil clusters that could have deleterious effects on protein translation. Although lower uracil content is desirable, in certain aspects, the uracil content, and in particular the local uracil content, of some subsequences of the wild-type sequence can be greater than the wild-type sequence and still maintain beneficial features (e.g., increased expression).

Preferably, the uracil-modified sequence induces a lower Toll-Like Receptor (TLR) response when compared to the wild-type sequence. Several TLRs recognize and respond to nucleic acids. Double-stranded (ds)RNA, a frequent viral constituent, has been shown to activate TLR3. Single-stranded (ss)RNA activates TLR7. RNA oligonucleotides, for example RNA with phosphorothioate internucleotide linkages, are ligands of human TLR8. DNA containing unmethylated CpG motifs, characteristic of bacterial and viral DNA, activate TLR9.

As used herein, the term “TLR response” is defined as the recognition of single-stranded RNA by a TLR7 receptor, and preferably encompasses the degradation of the RNA and/or physiological responses caused by the recognition of the single-stranded RNA by the receptor. Methods to determine and quantify the binding of an RNA to a TLR7 are known in the art. Similarly, methods to determine whether an RNA has triggered a TLR7-mediated physiological response (e.g., cytokine secretion) are well known in the art. Preferably, a TLR response can be mediated by TLR3, TLR8, or TLR9 instead of TLR7. Suppression of TLR7-mediated response can be accomplished via nucleoside modification. RNA undergoes over a hundred different nucleoside modifications in nature. Human rRNA, for example, has ten times more pseudouracil ('P) and 25 times more 2′-O-methylated nucleosides than bacterial rRNA. Bacterial mRNA contains no nucleoside modifications, whereas mammalian mRNAs have modified nucleosides such as 5-methylcytidine (m5C), N6-methyladenosine (m6A), inosine and many 2′-O-methylated nucleosides in addition to N7-methylguanosine (m7G).

Preferably, the uracil content of polynucleotides disclosed herein and preferably polynucleotides encoding the modified therapeutic protein of interest is less than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 7%1, 16%, 15%, 4%1, 3%1, 12%, 11%, 10%, 90%, 80%, 70%, 60%, 5%, 4%, 3%, 2% or 1% of the total nucleobases in the sequence in the reference sequence. Preferably, the uracil content of polynucleotides disclosed herein, is between about 5% and about 25%. Preferably, the uracil content of polynucleotides disclosed herein is about 15% and about 25%.

The cDNA templates may be transcribed to produce an mRNA sequence described herein using an in vitro transcription (IVT) system. The system typically comprises a transcription buffer, nucleotide triphosphates (NTPs), an RNase inhibitor and a polymerase. The NTPs may be selected from, but are not limited to, those described herein including natural and unnatural (modified) NTPs. The polymerase may be selected from, but is not limited to, T7 RNA polymerase, T3 RNA polymerase and mutant polymerases such as, but not limited to, polymerases able to incorporate modified nucleic acids.

The primary cDNA template or transcribed mRNA sequence may also undergo capping and/or tailing reactions. A capping reaction may be performed by methods known in the art to add a 5′ cap to the 5′ end of the primary construct. Methods for capping include, but are not limited to, using a Vaccinia Capping enzyme (New England Biolabs, Ipswich, Mass.) or CLEANCAP® technology (TriLink Biotechnologies). A poly-A tailing reaction may be performed by methods known in the art, such as, but not limited to, 2′ O-methyltransferase and by methods as described herein. If the primary construct generated from cDNA does not include a poly-T, it may be beneficial to perform the poly-A-tailing reaction before the primary construct is cleaned.

Codon optimized cDNA constructs encoding a modified therapeutic protein of interest are particularly suitable for generating mRNA sequences described herein. For example, such cDNA constructs may be used as the basis to transcribe, in vitro, a polyribonucleotide encoding a modified protein of interest. Table 6 provides a listing of exemplary cDNA ORF templates used for in vitro transcription of the mRNA sequences listed in Table 5.

TABLE 6
Exemplary cDNA Templates

| DNA Construct | SEQ | Protein encoded by |
|---|---|---|
| No***: | ID NO: | cDNA template SEQ ID NO: |
| p2260 | 221 | SEQ ID NO: 4** |
| p2262 | 221 | SEQ ID NO: 4** |

**SEQ ID NO: 4 is the amino acid sequence for modified human OTC.
***The entire plasmid sequence is not included.

The present disclosure also provides polynucleotides (e.g. DNA, RNA, cDNA, mRNA) encoding a modified human protein of interest that may be operably linked to one or more regulatory nucleotide sequences in an expression construct, such as a vector or plasmid. In certain embodiments, such constructs are DNA constructs. Regulatory nucleotide sequences will generally be appropriate for a host cell used for expression. Numerous types of appropriate expression vectors and suitable regulatory sequences are known in the art for a variety of host cells.

Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the embodiments of the present disclosure. The promoters may be either naturally occurring promoters, or hybrid promoters that combine elements of more than one promoter.

An expression construct may be present in a cell on an episome, such as a plasmid, or the expression construct may be inserted in a chromosome. Preferably, the expression vector contains a selectable marker gene to allow the selection of transformed host cells. Selectable marker genes are well known in the art and will vary with the host cell used.

The present disclosure also provides expression vectors comprising a nucleotide sequence encoding a modified protein of interest that is preferably operably linked to at least one regulatory sequence. Regulatory sequences are art-recognized and are selected to direct expression of the encoded polypeptide.

Accordingly, the term regulatory sequence includes promoters, enhancers, and other expression control elements. The design of the expression vector may depend on such factors as the choice of the host cell to be transformed and/or the type of protein desired to be expressed.

The present disclosure also provides a host cell transfected with an mRNA or DNA described herein which encodes a modified polypeptide described herein. For example, the modified polypeptide has the sequence of SEQ ID NO: 4. The host cell may be any prokaryotic or eukaryotic cell. For example, a modified polypeptide described herein may be expressed in bacterial cells such as E. coli, insect cells (e.g., using a baculovirus expression system), yeast, or mammalian cells. Other suitable host cells are known to those skilled in the art.

The present disclosure also provides a host cell comprising a vector comprising a polynucleotide which encodes an mRNA sequence of any one of SEQ ID NOs: 26-229.

The present disclosure also provides methods of producing a modified therapeutic human protein of interest. For example, a host cell transfected with an expression vector encoding a modified therapeutic protein of interest can be cultured under appropriate conditions to allow expression of the polypeptide to occur. The polypeptide may be secreted and isolated from a mixture of cells and medium containing the polypeptides. Alternatively, the polypeptides may be retained in the cytoplasm or in a membrane fraction and the cells harvested, lysed and the protein isolated. A cell culture includes host cells, media and other byproducts. Suitable media for cell culture are well known in the art.

The expressed modified therapeutic proteins described herein can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the modified polypeptide.

Therapeutic Proteins

The modified protein described herein can be a modified therapeutic protein. Any therapeutic human wild-type protein of interest is suitable for modification as described herein. In some embodiments, the therapeutic protein can be an intracellular antibody. Exemplary therapeutic proteins include, but are not limited to, modified therapeutic proteins of the present disclosure that have the activity of the following human wild-type proteins:

AIBG; AICF; A2M; A2MLI: A4GNT; AAAS; AACS: AADAC; AAGAB; AAKI; AANAT; AARD; AARS2; AARS; AASDH; AASDHPPT;

AASS; AATF; AATK; ABAT; ABCAI2; ABCAI3; ABCAI; ABCA2; ABCA3; ABCA5; ABCAB; ABCA7; ABCA9; ABCBII; ABCBI; ABCB4; ABCB5;

ABCB6; ABCB7; ABCB8; ABCB9; ABCCIO; ABCCII; ABCCI2; ABCCI; ABCC2; ABCC3; ABCC4; ABCC5; ABCC6; ABCC8; ABCC9; ABCDI;

ABCD2; ABCD3; ABCD4; ABCEI; ABCFI; ABCF2; ABCGI; ABCG2; ABCG4; ABCG5; ABCG8; ABHDI2B; ABHDI2; ABHDIBA; ABHDI7B; ABHD2;

ABHD5; ABHDB; ABII; ABI2; ABI3BP; ABI3; ABU; ABL2; ABLIMI: ABLIM2; ABO; ABRA; ABR; ABTI; ACAAI; ACAA2; ACACA; ACACB5; ACADIO; ACAD8; ACAD9: ACADL; ACADSB; ACADS; ACADVL; ACAN; ACATI; ACAT2; ACBD3; ACBD4; ACBD5; ACBDB; ACCS; ACD; ACE2;

ACE; ACER2; ACER3; ACHE; ACINI; ACKRI; ACKR2; ACKR3; ACKR4; ACLY; ACMSD; ACDI: AC02; ACOTI3; ACOTI; ACDT2; ACOT9; ACDXI;

AC0X2; AC0X3; AC0XL; ACPI; ACP2; ACP5; ACP6; ACPP; ACRBP; ACRC; ACR; ACVI; ACSBGI; ACSBG2; ACSF3; ACSLI; ACSL3; ACSL5;

ACSLB; ACSMI; ACSM2B; ACS3; ACSSI; ACSS2; ACSS3; ACTAI; ACTA2; ACTB; ACTBL2; ACTCI; ACTGI; ACTG2; ACTLBA; ACTLBB;

ACTL8; ACTL9; ACTNI; ACTN2; ACTN3; ACTN4; ACTRIA; ACTRIB; ACTR2; ACTR3B; ACTR3; ACTR5; ACTRTI; ACVRIB; ACVRIC; ACVRIO; ACVR2A; ACVR2B; ACVRLI; ACYI; ACY3; ACYP2; ADADI; ADA; ADAMIO; ADAMII; ADAMI2; ADAMI5; ADAMI7; ADAMI9; ADAM20; ADAM2I;

ADAM22; ADAM23; ADAM28; ADAM29; ADAM2; ADAM33; ADAM7; ADAM8; ADAM9; ADAMDECI: ADAMTSIO; ADAMTSI2; ADAMTSI3;

ADAMTSI4; ADAMTSI5; ADAMTSIB; ADAMTSI7; ADAMTSI8; ADAMTSI9; ADAMTSI; ADAMTS20; ADAMTS2; ADAMTS3; ADANTS4;

ADAMTS5; ADAMTSB; ADAMTS7; ADAMTSB; ADAMTS9; ADAMTSLI; ADAMTSL2; ADAMTSL3; ADAMTSL4; ADAMTSL5; ADAPI; ADAP2;

ADARBI; ADARB2; ADAR; ADAT2; ADAT3; ADCK3; ADCK4; ADCYIO; AOCYI; ADCY2; ADCY3; ADCY5; ADCYB; ADCY7; ADCY8; ADCY9; ADCYAPI; ADCYAPIRI; ADD1; ADD2; ADD3; ADGB; ADGRAI; ADGRA2; ADGRA3; ADGRBI; ADGRB2; ADGRB3; ADGRDI; ADGRD2; ADBREI; ADGREZ; ADGRE5; ADGRF5; ADGRGI; ADGRGZ; ADGRG3; ADGRGB; ADGRG7; ADGRL1; ADGRL3; ADGRVI; AOHIA; ADHIB; ADH4; ADH5;

ADHB; ADH7; ADHFEI; AOIt; AOIG; ADIPBD; ADIPBRI; ADIPDR2; ADIRF; ADK; ADM2; ADM; ADNP; ADO; ADDRAI; ADORA2A; ADORA2B;

ADPGK; ADPRH; ADPRHL1; ADPRHL2; ADRAIA; ADRAIB; ADRAID; ADRA2A; ADRA2B; ADRA2C; ADRBI; ADRB2; ADRB3; AORB1; ADRBK2;

ADRMI; ADSL; ADSS; ADSSLI; ADTRP; AEBP2; AEN; AES; AFAPI; AFAPIL; AFAPIL2; AFFI; AFF2; AFF3; AFF4; AFG3L2; AF; AFP; AGA; AGAPt; AGAP2; AGAP3; AGAP4; AGBLI; AGBL2; AGBL3; AGBL4; AGER; AGFGI; AGFG2; AGGFI; AG; AGMD; AGD2; ABPATI AGPAT2;

AGPAT3; AGPAT4; AGPAT9; AGPS; AGR2; AGR3; ABRN; AGRP; AGT; AGTPBPI; AGTRI; AGTR2; AGXT2; AGXT; AHCY; AHCYL2; AHII;

AHNAK; AHR; AHRR; AHSAI; AHSA2; AHSG; AICDA; AIDA; AIR: AIFMI AIFM2; AIFM3; AIGI AIM1: AIM2; AIP1; AIMP2; AIP: AIPLI; AIRE;

AJAPI; AJUBA; AI; A3; AB; AK7; AK8; AK9; AKAPID; AKAPI2; AKAPI3; AKAPI7A; AKAPI; AKAP2; AKAP3; AKAP4; AKAP5; AKAPB;

AAP7; AKAPB; AIPI AKIRIN2; ANA; AKRIBID; AKRIBI; ACI: AKRIC2; AKRIC3; AKRIC4; AKRIDI; AKRIE2; AKR7A3; AKTI; AKTISI; ID AKT2; AKT3; AKTIP; ALAD; ALASI; ALAS2; ALB; ALCAM; ALDHIBAI: ALDHI8AI: ALDHIAI ALDHIA2; ALDHIA3; ALDHIBI; ALDHIU; ALDH2;

ALDH3AI; ALDH3A2; ALDH3BI ALDH4AI; ALDH5AI: ALDHBAI: ALDH7AI: ALDH9AI ALDDA; ALDDB; ALDDC; ALGIOB; ALGIQ; ALGII; ALGI2;

ALGI3; ALGI; ALGIL; ALG2; ALG3; ALG8; ALG9; ALKBHI; ALKBH2; ALKBH3; ALKBH7; ALKBH8; ALK; ALLC; ALMSI; ALDXI2B; ALDXI2;

AL0XI5B; ALDXI5; ALDX5AP; AL0X5; ALDXE3; ALPI ALPKI; ALPK2; ALPK3; ALPP; ALPPL2; ALS2CL; ALS2CRI2; ALS2; ALXI; ALX4;

ALYREF; AMACR; AMBN; AMBP; AMBRAI; AMDI; AMELX; AMELY; AMERI; AMER2; AMER3; AMFR; AMH; AMHR2; AMICAI; AMIGD2; 15 AMMECRI; AMN; AMOT; AMDTLI: AMPD2; AMPD3; AMPH; AMT; AMZI; ANAPCIO; ANAPCII; ANAPCI3; ANAPCI; ANAPC2; ANAPC4; ANAPC5;

ANAPC7; ANG; ANGPTI; ANGPT2; ANGPT4; ANGPTLI ANGPTL2; ANGPTL4; ANGPTLG; ANKI; ANK2; ANK3; AN FNI ANKFYI: ANKHDI-EIF4EBP3; ANKHD1; ANKH; ANKKt; ANKLEI; ANKLE2; ANKMYI; ANKRDIO; ANKRDII; ANKRDI2; ANKRDI8A; ANKRDI; ANKRD23; ANKRD2B;

ANKRD28; ANKRD2; ANKRD3DA; ANKRD3BB; ANKRD3B; ANKRD37; ANKRD44; ANKRD45; ANKRD4B; ANK D5D; ANKRD55; ANKRDB;

ANKRD7; ANKSIA; ANKSIB; ANKS4B; ANKSB; ANLN; ANDID; ANDI; AN02; AN03; AND4; AND5; AN06; AND7; ANP32A; ANP32B; ANP32D; 0 ANPEP; ANTXRI; ANTXR2; ANXAID; ANXAII; ANXAI3; ANXAI ANXA2; ANXA2R; ANXA3; ANXA4; ANXA5; ANXAB; ANXA7; ANXA8; ANXABLI;

ADAH; AOCI; AOC2; AOC3; ADXI: APIAR; APIBI; APIGI; APIMI; APIM2; APISI; APIS2; APIS3; AP2AI; APZBI; AP2MI; AP2SI; AP3BI; AP3DI;

AP3M2; AP3SI; AP3S2; AP4BI; AP4EI; AP4MI; AP4SI AP5MI: AP5ZI; APAFI; APBAI; APBA2; APBA3; APBBI; APBBIIP; APBB2; APBB3;

APC2; APCDDI; APCDDIL; APC; APCS; APEH; APEXI; APEX2; APHIB; APIS; APIP; APLN; APLNR; APLPI; APLP2; APDAIBP; AP0A2; AP0A4;

APDA5; APDBECI; APDBEC2; APDBEC3AJ; APOBEC3B; APDBEC3C; APDBEC3F; APDBEC3G; APOBEC3H; APDB; APOBR; AP0C5; APDC2; AP0C3; AP0C4; APDD; APDE; APDF; APDH; APDLI: APDL2; APDL3; APOL4; APOLB; APOLDI: APDM; APDD; APOPTI APPBP2;

APP; APPLI; APPL2; APRT; APTX; AflPID; ADPI; AOP2; ADP3; ADP4; ADP5; ADPB; ADP7; ADP8; ADP9; ARAF; ARAPI; ARAP3; ARC;

ARCNI; AREG; ARFI; ARF3; ARF4; ARFB; AFGAPI; ARFGAP2; ARFGAP3; ARFGEFI; ARFGEF2; ARFGEF3; AFiPf; ARFRPI: ARB1; ARGLUI;

ARHGAPID; ARHGAPIIA; ARHGAPIIB; ARHGAPI5; ARHGAPI8; ARHGAPI; ARHGAP2D; ARHGAP2I; ARHGAP22; ARHGAP23; ARHGAP24; ARHGAP25; ARHGAP2B; ARHGAP27; ARHGAP28; ARHGAP30; ARHGAP3I; ARHGAP32; ARHGAP35; ARHGAP42; ARHGAP4; ARHGAP5; D ARHGAPB; ARHGAP9; ARHGDIA; ARHGDIB; ARHGEFIO; ARHGEFIDL; ARHGEFII: ARHGEFI2; ARHGEFI5; ARHGEFIB; ARHGEFI7; ARHGEFI:

ARHGEF25; ARHGEF2B; ARHGEF28; ARHGEF2; ARHGEF38; ARHGEF3; ARHGEF4; ARHGEF5; ARHGEFB; ARHGEF7; ARHGEF9; AR; ARIDIB;

ARID2; ARID3A; ARID3B; ARID4A; ARID4B; ARID5B; ARIHI; ARUI; ARLI3A; ARLI3B; ARLI4EP; ARLI4; ARLI5; ARLI; ARL2BP; ARL2; ARL3;

ARL4A; ARL4C; ARL4D; ARL5A; ARL5B; ARLB; ARLBIPI; ARLBIP5; ARMCIO; ARMCI ARMC2; ARMC3; ARMC4; ARMC5; ARMC8; ARMC9;

ARMCXI; ARMS2; ARMTI: ARNT2; ARNT; ARNTL2; ARNTL; ARPCIA; ARPCIB; ARPC2; ARPC3; ARPC5; ARPIN; ARPP2I; ARR3; ARRBI; 5 ARRB2; ARRDC2; ARRDC3; ARRDC4; ARSA; ARSB; ARSD; ARSE; ARSF; ARSG; ARSH; ARSI: ARSJ; ARSK; ARTI; ART3; ART4; ARVCF; ARX; AS3MT; ASAHI; ASAH2; ASAPI; ASAP2; ASBID; ASBI3; ASBI5; ASBIS; ASBI; ASB2; ASBB; ASB7; ASCCI; ASCC2; ASCC3; ASCLI;

ASCL2; ASCL4; ASFIA; ASFIB; ASHIL; ASH2L; ASICI; ASIC2; ASIC3; ASIC4; ASICS; ASIP; ASL; ASMT; ASMTL; ASNAI; ASMS; ASPA; ASPG;

ASPH; ASPM; ASPN; ASPRVI; ASPSCRI; ASRBLI; ASSI ASTN2; ASUN; ASXLI; ASXL2; ASXL3; ASZI; ATAD2; ATAD3B; ATAD3C; ATAD5;

ATATI; ATCAY; ATEI; ATFI; ATF2; ATF3; ATF4; ATF5; ATFBB; ATFB; ATF7; ATF7IP; ATBIO; ATGI2; ATGIBLI: ATG2B; ATG3; ATG4A; ATG4B; ATG4C; ATG5; ATG7; ATG9A; ATIC; ATLI; ATL2; ATL3; ATM; ATNI; ATDHI; ATDH7; ATOXI; ATPIDA; ATPIOB; ATPIOD; ATPIIA; ATPIIAUN;

ATPIIB; ATPI2A; ATPI3A3; ATPI3A4; ATPIA2; ATPIA3; ATPIA4; ATPIBI; ATPIB2; ATP2AI; ATP2A2; ATP2A3; ATP2BI; ATP2B2; ATP2B3; ATP2B4; ATP2CI; ATP2C2; ATP4A; ATP4B; ATP5AI; ATP5B; ATP5CI; ATP5D; ATP5E; ATP5GI; ATP5G2; ATP5G3; ATP5H; ATP5J2; ATP5J;

ATP5L; ATP5D; ATPBAPI; ATPBAPIL; ATPBAP2; ATP6V0AI; ATPBV0A2; ATPBV0A4; ATPBVOC; ATPBVDDI; ATPBVOEI; ATPGVOE2;

ATPBVIBI; ATPGVIB2; ATPBVICI; ATPBVIO; ATPBVIEI; ATPBVIF; ATPBVIGI; ATPBVIG2; ATPBVIG3; ATP7A; ATP7B; ATP8AI; ATP8A2; ATP8BI; ATP8B3; ATP8B4; ATP9B; ATPAF2; ATRAID; ATR; ATRIP; ATRN; ATRNLI; ATRX; ATXNIO; ATXNI; ATXNIL; ATXN2; ATXN2L; ATXN 3;

ATXN7; ATXN7L3B; AUH; AURKA; AU KB; AURKC; AUTS2; AVEN; AVP; AVPII; AVPRIA; AVPRIB; AVPR2; AWATI; AXDNDI; AXINI: AXIN2;

AXL; AZGPI; AZI2; AZINI; AZIN2; AZUI; B2M; B3GALNTI; B3GALNT2; B3GALT2; B3GALT4; B3GALT5; B3GALTL; B3GATI; B3GAT2; B3GAT3;

B3GNT2; B3GNT3; B3GNT5; B3GNTB; B3GNT8; B3GNTLI; B4GALNTI; B4GALNT2; B4GALNT3; B4GALTI; B4GALT3; B4GALT4; B4GALT5;

B4GALTB; B4GALT7; B4GATI; B9DI: B9D2; BAALC; BAAT; BABAMI; BACEI; BACE2; BACHI; BACH2; BAD; BAG); BAG3; BAG4; BAGS; BAGB; BAIAP2LI; BAIAP3; BAKU BAMBI: BANFI BANKI; BANP; BAPI; BARDI; BARHLI; BARXI: BARX2; BASPI; BATF2; BATF; BAX; BAZIA; BAZIB;

BAZ2A; BAZ2B; BBC3; BBIPI; BBOXI; BBSID; BBSI2; BBS1; BBS2; BBS4; BBSS; BBS7; BBSS; BBX; BCAM; BCAN; BCAP29; BCAP3I;

BCARI; BCASI; BCAS3; BCAS4; BCATI; BCAT2; BCCIP; BCDIN3D; BCHE; BC DHA; BC DHB; BC DK; BCLIO; BCLIIA; BCLIIB; BCL2AI;

BCL2; BCL2LIO; BCL2LII; BCL2LI2; BCL2LI3; BCL2LI4; BCL2LI; BCL2L2; BCL2L2-PABPNI; BCL3; BCLBB; BCLB; BCL7A; BCL7B; BCL7C;

BCL9L; BCLAFI; BCDI; BC02; BCDR; BCORLI; BCR; BCSIL; BDHI; BDH2; BDKRBI; BDKRB2; BDNF; BDPI; BEANI: BEGAIN; BENDS; BEND4; BESTI; BEST2; BETI; BETIL; BEXI; BEX2; BEX4; BFAR; BFSPI; BFSP2; BGLAP; BGN; BHLHAI5; BHLHA9; BHLHB9; BHLHE22; BHLHE23;

BHLHE40; BHLHE4I; BHMT2; BHMT; BICCI; BICDI; BICD2; BID; BINI: BIN2; BINS; BIRC2; BIRC3; BIRC5; BIRCB; BIRC7; BIVM; BLCAP;

BUD; BLK; BLMH; BLM; BLNK; BLOCIS2; BLDCIS3; BLOCIS4; BLOCIS5; BLDCISB; BLVRA; BLVRB; BLZFI: BMF; BMII BMPIO; BMPI5;

BMPI; BMP2; BMP2K; BMP3; BMP4; BMP5; BMPG: BMP7; BMP8B; BMPER; BMPRIA; BMPRIB; BMPR2; BMSI; BMX; BNCI; BNC2; BNIPI;

BNIP2; BNIP3; BNIP3L; BNIPL; BDC; BODIL2; BO; BOLA3; BDLL; BDPI: BORA; BPGM; BPIFAI; BPIFA2; BPIFA3; BPIFBI BPIFB2; BPIFC; BPI; BPNTI; BPTF; BPY2; BRAF; BRAP; BRATI; BRCAI; BRCA2: BRCC3; BRDI; BRD2; BRD3; BRD4; BRD7; BRD8; BRE; BRFI; BRF2;

BRI3BP; BRI3; BRINPI; BRINP2; BRINP3; BRIPI; BRKI; BRMSI; BRMSIL; BRS3; BRSI; BRSK2; BRWDI: BRWD3; BSCL2; BSG; BSN;

BSPHI; BSPRY; BSTI; BST2; BSX; BTBDID; BTBDII; BTBDIB; BTBDI; BTBD2; BTBD3; BTBD9; BTC; BTD; BTF3; BTGI; BTG2; BTG3; BTG4;

BTK; BTLA; BTNIAI; BTN2AI; BTN2A2; BTN3AI; BTN3A2; BTN3A3; BTNL2; BTRC; BUBIB; BUBI; BUB3; BUDI3; BUD3I; BVES; BYSL;

BZRAPI; BZWI; CID; CIGALTICI; CIGALTI;

CIOB; CIDBP; CIQLI; CIDL3; CIQTNFI; CIQTNF3; C1QTNF5: CIOTNFB; CIOTNF7; CIDTNF9B-ASI; CIR: CIRL; CIS; C2CO3; C2CD4A; C2CD4B; C2CD5; C2; C4A; C4B2; C4B; C4BPA; C4BPB; C5ARI:

C5AR2; C5; CB;

CAIO; CAM; CAI2; CAI3; CAI; CA2; CA3; CA4; CA5A; CAB; CA8; CAB39; CAB39L; CABINI; CABLES!; CABP2; CABP4; CABSI;

CABYR; CACNAIA; CACNAIB; CACNAIC; CACNAID; CACNAIE; CACNAiF: CACNAIG; CACNAIH; CACNAII: CACNAIS; CACNA2DI; CACNA2D2;

CACNA2D3; CACNA2D4; CACNBI; CACNB2; CACNB3; CACNB4; CACNG2; CACNG3; GACNG4; CACNG5; CACNGB; CACULI; CACYBP; CAD; ID CADMI; CADM2; CADM3; CADM4; CADPS2; CADPS; GAGEI; CALBI; CALB2; CALCA; CALCB; CALCOCDI; CALCDCD2; CALCR; CALCRL;

CALDI; CALHMI; CALHM2; CALHM3; CALM2; CALML3; CALML5; CALNI: CALR3; CALR; CALU; CALY; CAMKID; CAMKIG; CAMKI; CAMK2A;

CAMK2B; CAMK2D; CAMK2G; CAMK4; CAM KI: CAMKK2; CAMKMT; CAMLG; CAMP; CAMSAPI; CAMSAP2; CA TAI; CANOI: CAND2; CANTI:

CANX; CAPI; CAP2; CAPG; CAPNID; CAPNI3; CAPNI4; CAPNI CAPN2; CAPN3; CAPN5; CAPNB; CAPN7; CAPN9; CAPNSI: CAPRINI;

CAPRIN2; CAPS2; CAPS; CAPSL; CAPZA2; CAPZA3; CARDID; CARDII; CARDI4; CARDIB; CARDB; CARDS: CARD9; CARF; CARKD; CARMI; 15 CARS; CARTPT; CASCI; CASC3; CASC4; CASC5; CASDI; CASK; CASPIO; CASPI4; CASPI; CASP2; CASP3; CASP4; CASP5; CASPB;

CASP7; CASP8AP2; CASPB; CASPB; CASDI CASR; CASS4; CAST: CASZI: CAT; CATSPERI; CATSPER2; CAVI; CAV2; CAV3; CBFA2T2;

CBFA2T3; CBFB; CBLB; CBL; CBLLI; CBLNI; CBLN2; CBLN4; CBRI; CBR3; CBR4; CBS; CBXI; CBX2; CBX3; CBX4; CBX5; CBXB; CBX7;

CBX8; CBYI; CC2DIA; CC2DIB; CC2D2A; CCARI; CCAR2; CCBEI; CCBLI; CCDCIDI; CCDCI02B; CCDCID3; CCDCID5; CCDCID8; CCDCII4;

CCDCII5; CCDCI2I; CCDCI22; CCDCI29; CCDCI3D; CCDCI34; CCDCI3B; CCDCI40; CCDCI4I; CCDCI48; CCDCI5I; CCDCIB7; CCDCIB9-D SOHLH2; CCDCI70; CCDCI7I; CCDCI75; CCDCI7B; CCDCI78; CCDCI8D; CCDCI8I: CCDCI85; CCDCI22; CCDCI28A; CCDCI39; CCDCI3;

CCDCI4D; CCDCI42B; CCDCI42; CCDCI5D; CCDCI54; CCDCBD; CCDCB2; CCDCB3; CCDCG5; CCDCBB; CCDCB7; CCDCB8; CCDCB;

CCDCI78; CCDCI8D; CCDCI83; CCDCI85A; CCDCBB; CCDCI88A; CCDCI88C; CCDCI8; CCDCI9I; CCDCI94; CCDCI97; CCHCRI; CCKAR; CCKBR;

CCK; CGLII; CCLI3; CCLI4; CCLI5; CCLIB; CCLI7; CCLI8; CCLI9; CCD; CCL2D; CCLZI: CCL22; CCL23; CCL24; CCL25; CCL2G; CCL27;

CCL28; CCL2; CCL3; CCL3L3; CCL4; CCL4LI; CCL4L2; CCL5; CCL7; CCL8; CCM2; CCNAI; CCNA2; CCNBI; CCNB2; CCNB3; CCNC; CCNDt:5 CCND2; CCND3; CCNDBPI: CCNEI; CCNE2; CCNF; CCNBI: CCNG2; CCNH; CCNI; CCNJ; CCNJL; CCNK; CCNL1; CCNL2; CCNO; CCNTI;

CCNY; CCPIID; CCRID; CCRI; CCR2; CCR3; CGR4; CCR5; CCRB; CCR7; CCR8; CCR9; CCRL2; CCRN4L; CCSERI; CCS; CCT2; CCT3; CCT4;

CCT5; CCTGA; CCTBB; CCT7; CDI09; CDI4; CDI5I; CDIB3; CDIB3LI; CDIB4; CDI77; CDI8D; CDI9; CDIA; CDIB; CDIC; CDID; CDIE; CD20D;

CD2DDRI; CD2D7; CD2D9; CD22B; CD22; CD244; CD247; CD248; CD24; CD274; CD27B; CD27; CD28; CD2AP; CD3DDA; CD3DOC;

CD3D0LF; CD302; CD320; CD33; CD34; CD3B; CD37; CD38; CD3D; CD3EAP; CD3E; CD3G; CD40; CD4DLG; CD44; CD4B; CD47; CD48; D CD4; CD55; CD59; CD5; CD5L; CDB3; CDG8; CDB9: CDB; CD72; CD74; CD79A; CD79B; CD7; CD8D; CD8I; CD82; CD83; CD84; CD8B;

CD8A; CD8B; CD93; CD9B; CD99; CDB; CDADCI: CDANI; CDCI23; CDCI4A; CDCI4B; CDCIG; CDCI2DB; CDCI23; CDCI25A; CDCI25B;

CDCI25C; CDCI27; CDCI34; CDCI37; CDCI37LI; CDCI42BPA; CDCI42BPB; CDCI42BPG; CDCI4ZEPI: CDCI42EP3; CDCI42SE2; CDCI45; CDCI5L;

CDCB; CDCI73; CDCI7; CDCA2; CDCA3; CDCA5; CDCA7; CDCA7L; CDCPI; CDHID; CDH11: CDHI3; CDHI5; CDHIB; CDHI7; CDHI8; CDHI9;

CDHI; CDH2D; CDH22; CDH23; CDH2B; CDH2; CDH3; CDH4; CDH5; CDHB; CDH7; CDH8; CDH9; CDHRI; CDHR2; CDHR3; CDHR5; CDIPT; 5 CDKID; CDKIIB; CDKI2; CDKI3; CDKI4; CDKI5; CDKIB; CDKI7; CDKI8; CDKI9; CDKI; CDK2D; CDK2API; CDK2AP2; CDK2; CDK3; CDK4; C0K5; CDK5 I: CDK5R2; CDK5RAPI: CDK5RAP2; CDK5 AP3; CDKB; CDK7; CDK8; CDK9; CDKALI; CDKLI CDKL2; C0KL3; CDKL4;

CDKL5; CDKNIA; CDKNIB; CDKNIC; CDKN2A; C0KN2A1P: CDKN2B; CDKN2C; CDKN2D; CDKN3; CDNF; COOI; CDON; CDRI; CDR2; CDS2;

CDTI: CDV3; CDXI CDX2; CDX4; GDYIB; CDY2B; CDYL2; CDYL; CEACAMIB; CEACAMIB; CEACAMI; CEACAM2I; CEACAM3; CEACAM4;

CEACAM5; CEACAMB; CEACAM7; CEACAM8; CEBPA; CEBPB; CEBPD; CEBPE; CEBPG; CEBPZ; CECRI; CECR2; CELAI: CELA3B; CELFI; CELF2; CELF4; CELF5; CELFB; GEL; CELSRI; CELSR3; CEMIP; CEMPI; CENDI; CENPA; CENPB; CENPC; CENPE; CENPF; CENPH; CENPJ;

CENPK; CENPN; CENPO; CENPO; CENPU; CENPV; CENPW; CEPII2; CEPI2D; CEPI28; CEPI3I; CEPI35; CEPI52; CEPIB2; CEPIB4; CEPI7D;

CEPI92; CEPIB; CEP25D; CEP29D; CEP4I; CEP55; CEP57; CEPB3; CEPG8; CEP72; CEP7B; CEP83; CEP85L; CEP83; CERI; CERK; CERKL;

CERSI; CERS2; CERS3; CERS4; CERSB; CESh CES2; CES3; CETNI; CETN2; CETN3; CETP; CFAPI2B; CFAP3B; CFAP44; CFAP52; CFAP53;

CFAP57; CFAP58; CFAPBI; CFAPBB; CFAP97; CFB; CFCIB; CFCI; CFD; CFDPI; CFH; CFHRI; CFHR2; CFHR3; CFHR4; CFH 5; CFI; CFLI; CFL2; CFLAR; CFP; CFTR; CGA; CGBI; CGB2; CGB5; CGB7; CBN; CGNLI; CGRRFI: CH25H; CHACI; CHAD; CHAFIA; CHAFIB; CHAMPI CHAT;

CHCHDIO; CHCHDI; CHCHD3; CHCHD5; CHCHDB; CHCHD7; CHDI; CHD2; CHD3; CHD4; CHDB; CHD7; CHD8: CHDH; CHEKI; CHEK2;

CHERP; CHFR; CHGA; CHGB; CHI3LI; CHICI; CHIC2; CHITI; CHKA; CHKB; CHLI; CHM; CHML; CHMPIA; CHMPIB; CHMP2B; CHMP3;

CHMP4A; CHMP4B; CHMP4C; CHMP5; CHMI: CHN2; CHDDL; CHORDCI; CHPI: CHP2; CHPF; CHPTI CHRACI; CHRD; CHRDLI: CHRFAM7A;

CHRMI; CHRM2; CHRM3; CHRM4; CHRM5; CHRNAIO; CHRNAI; CHRNA2; CHRNA3; CHRNA4; CHRNA5; CHRNAB; CH NA7; CHRNBI; CHRNB2; CHRNB3; CHRNB4; CHRND; CHRNE; CHRNG; CHSTIO; CHSTII; CHSTI2; CHSTI3; CHSTI4; CHSTI5; CHSTI; CHST2; CHST3;

CHST4; CHST5; CHSTB; CHST8; CHST9; CHSYI; CHSY3; CHTFI8; CHTOP; CHURCI; CIADI; CIAPINI; CIBI; CIB2; CIC; CIDEB; CIDEC; CIITA;

CILP2; CILP; CINP; CIPC; GIRL CIRBP; CIRHIA; CISDI; CISD2; CISH; CITEDI; CITED2; CIT; CIZI: CKAP2; CKAP2L; CKAP4; CKAP5; CKB;

CKLF; CKM; CKMTIA; CKMTIB; CKMT2; CKSIB; CKS2; CLASPI; CLASP2; CLCA2; CLCA4; CLCFI; CLC; CLCNI; CLCN2; CLCN3; CLGN4;

CLCN5; CLCNB; CLCN7; CLCNKB; CLDNID; CLDNII; CLDNI4; CLDNI5; CLDNIB; CLDNI8; CLDNI; CLDN23; CLDN2; CLDN5; CLDNB; CLDN7; CLDN8; CLDNB; CLECIDA; CLECIIA; CLECI2A; CLECI4A; CLECIBA; CLECIB; CLEC2A; CLEC2B; CLEC2D; CLEC2L; CLEC3B; CLEC4A;

CLEC4C; CLEC4D; CLEC4E; CLEC4G; CLEC4M; CLEC5A; CLECBA; CLEC7A; CLEC9A; CLECLI; CLGN; CLICI; CLIC3; CLIC5; CLICB; CLINTI;

CLIP1; CLIP2; CLKl; CLK2; CLLUI; CLMN; CLMP; CLN3; CLN5; CLNB; CLNB: CLNK; CLNSIA; CLOCK; CLPI; CLPP; CLPS; CLPTMI; CLPTMIL;

CLRNI; CLSPN; CLSTNI; CLSTN2; CLTA; CLTC; CLTCLI: CLUAPI; CLU; CLULI; CLVSI: CLYBL; CMAI; DMAS; CMCI; CMC2; CMC4; CMIP;

CMKLRI; CMPK2; CMSSI; CMTM3; CMTM5; CMTM7; CMTM8; CMTRI; CMTR2; CMYA5; CNBDI; CNBP; CNDPI; CNDP2; CNGAI; CNGA3; CNGBI; CNGB3; CNIH3; CNKSRI; CNKSR2; CNKSR3; CNNI; CNN2; CNNMI: CNNM2; CNNM4; CNQTI; CNBT2; CNDT3; CN0T4; CNDTB;

CNOTGL; CN0T7; CN0T8; CNP; CNPY2; CNPY3; CNRI: CNR2; CNRIPI: CNST; CNTF; CNTFR; CNTLN; CNTNI; CNTN2; CNTN3; CNTN4;

CNTN5; CNTNB; CNTNAPI; CNTNAP2; CNTNAP3; CNTNAP4; CNTNAP5; CNTRL; CNTROB; COAI; COA5; CDASY; COBL; COBLLI; COCH;

COGI; COG2; COG3; COG4; COG5; COGB; COG7; COG8; COIL; COLIDAI; COLIIAI; COLIIA2; COLI2AI; COLI3AI; COLI4AI; COLI5AI; COLIBAI;

COLI7AI; COLI8AI; COLIAI; COLIA2; COL2OAI; COL2IAI; COL22AI; COL23AI: COL24AI; COL25AI; COL2BAI; COL27AI; COL28AI; COL2AI; COL3AI; COL4AI; COL4A2; COL4A3BP; COL4A3; COL4A4; COL4A5; COL4AB; COL5AI; COL5A2; COL5A3; COLGAI; COLBA2; COLBA3;

COLBA5; COL7AI; COL8AI; COL8A2; COLGAI; COL9A2; COL9A3; COLCAI; COLCA2; COLECIO; COLECII: COLECI2; COLGALT2; COLQ;

COMMDIO; COMMDI; COMMD3-BMII; COMMD5; COMMD7; COMP; COMT; COPA; COPBI; COPB2; COPE; COPG2; COPRS; COPS2; COPS3;

COPS4; COPS5; COPSE; COPS7A; COPS8; COPZ2; COD2; COO3; COO4; COO5; COO6; COO7; COO9; CORIN; CORDIA; CORDIB; COROIC;

CORO2A; CORO2B; COROB; CORO7; CORO7-PAMIG; COTLI; COXIO; COXII; COXI4; COXI5; COMB; COXI7; COXI8; COX19; COX4II; COX4I2; COX5A; COX5B; COXBAI; COXBA2; COXBBI; COXGC; COX7AI; COX7A2; COX7A2L; COX7B2; COX7B; COX7C; COX8A; CPA1; CPA2; CPA3; CPA4; CPAB; CPAMD8; CPBI; CPB2; CPD; CPEBI; CPEB3; CPEB4; CPEOI; CPE; CP; CPLXI; CPLX2; CPLX3; CPLX4; CPM; CPNI; CPN2;

CPNEI; CPNE2; CPNE3: CPNE4; CPNE7; CPNE8: CPD; CPDX: CPPEOI; CPD; CPSI; CPSFI; CPSF2; CPSF3; CPSF3L; CPSF4; CPSFB;

CPSF7; CPTIA; CPTIB; CPTIC; CPVL; CPXCRI; CPZ; CRI; CR2; CRABPI; CRABP2; CRACR2A; CRADD; CRAMPIL; CRAT; C BI; CRB2; CRB3;

CRBN; CRCP; CRCTI; CREBI; CREB3; CREB3LI; CREB3L2; CREB3L3; CREB3L4; CREB5; CREBBP; CREBRF; CREBZF; CREGI; CRELDI; CRELD2; CREM; CRHBP; CRH; CRHRI: CRHR2; CRIMI; CRIP2; CRIP3; CRIPAK; CRIPT; CRISPI; CRISP2; CRISP3; CRISPLDI; CRISPLD2;

CRK; CRKL; CRLFI; CRLF2: CRLF3; CRLSI; CRMPI; CRNKLI: CRNN; CRDCC; CROT; CRP; CRTACI: CRTAM; CRTAP; CRTCI; CRTC2; CRTC3;

CRX; CRYI; CRY2; CRYAB; CRYBAI; CRYBA2; CRYBA4; CRYBBI; CRYBB2; CRYBB3; CRYGB; CRYGC; CRYGD; CRYGS; CRYLI CRYM;

CRYZLI; CSAD; CSAG3; CSEIL; CSFIR; CSF2; CSF2RA; CSF2RB; CSF3; CSGALNACTI; CSGALNACT2; CSHI; CSH2; CSHLI; CS; CSK;

CSMDI; CSMD2; CSMD3; CSNISI; CSN2; CSN3; CSNKIAI CSNKID; CSNKIG3; CSNK2AI CSNK2A2; CSNK2B; CSPG4; CSPG5; CSPPI; CSRNPI; CSRNP3; CSRPI CSRP2BP; CSRP2; CSRP3; CSTI; CST2; CST3; CST4; CST5; CSTB; CST7; CST8; CST9; CSTBL; CSTA; CSTB;

CSTFI; CSTF2; CSTF2T: CSTLI: CT45AI; CT55; CT83: CTAGIA; CTAG2; CTAGEI; CTAGE5; CTBPI; CTBP2; CTCI; CTCF; CTCFL; CTDP1:

CTDSPI; CTDSP2; CTDSPL; CTFI; CTGF; CTHRCI; CTIF; CTLA4; CTNNAI; CTNNA2; CTNNA3; CTNNALI; CTNNBt; CTNNBLI: CTNNDI;

CTNND2; CTNS; CTPSI; CTR9; CTRBI; CTRB2; CTRC; CTRL; CTSA; CTSB; CTSC; CTSD; CTSE; CTSF; CTSG; CTSH; CTSL; CTSD; CTSV;

CTSW; CTSZ; CTTNBP2; CTTN; CTUI; CTXN3; CUBN; CUEDCI; CUEDC2; CULL: CUL2; CUL3; CUL4A; CUL4B; CUL5; CUL7; CUL9; CUTA; CUXI; CUX2; CUZDI; CWC22; CWC27; CWFIBLI; CWFIBL2; CWH43; CX3CLI; CX3CRI; CXADR; CXCLIO; CXCLII: CXC LIZ; CXCLI3; CXCLI4;

CXCLIB; CXCLI7; CXCLI; CXCL2; CXCL3; CXCL5; CXCLB; CXCL8; CXCL9; CXCRI; CXCR2; CXCR3; CXCR4; CXCR5; CXCRB;

CXXCI; CXXC4; CYB5BID2; CYB5BI; CYB5A; CYB5B; CYB5R3; CYB5R4; CYBA; CYBB; CYBRDI: CYCI; CYCS; CYFIPI;

CYFIP2; CYGB; CYLCI; CYLC2; CYLD; CYP11A1; CYPIIBI: CYPIIB2; CYPI7AI; CYPI9AI; CYP1A1: CYPIA2; CYPIBI; CYP2IA2; CYP24AI;

CYP2BAI; CYP2BBI; CYP2GCI; CYP27AI; CYP27BI; CYP27CI: CYP2AI3; CYP2AB; CYP2A7; CYP2BB; CYP2CI8; CYP2CI9; CYP2C8; CYP2C9; CYP2DG; CYP2EI; CYP2FI; CYP2RI; CYP2SI; CYP2UI; CYP2WI; CYP39AI; CYP3A43; CYP3A4: CYP3A5; CYP3A7-CYP3A5IP;

CYP3A7; CYP4BAI; CYP4AII; CYP4BI; CYP4FII; CYP4FI2; CYP4F22; CYP4F2; CYP4F3; CYP4F8; CYP4V2; CYP5IAI; CYP7AI; CYP7BI;

CYP8BI; CYSI; CYSLTRI; CYTHI; CYTH3; CYTIP; CYTLI; CYYRI; D2HGDH; DAAMI: DAAM2; DAB2; DAB2IP; DACHI; DACH2; DACTI; DACT2;

DACT3; DADI: DAGI; DAND5; OAOA: DAD; DAPS; DAP; DAPKI: DAPK2; DAPK3; DARS2; DARS; DAWi DAXX; DAZI; DAZ2; DAZ3; DAZ4;

DAZAP1; DAZAP2; DAZL; DBF4; DBH; DBI; DBNI; DBNL; DBP; DBRI; DBXI; DCAFI2; DCAFI3; DCAFI7; DCAF4; DCAF5; DCAFB; DCAF7; DCAF8; DCANPI; DCBLDI: DCBLD2; DCC; DCDCI2C; DCDCI2; DCD; DCHSI; DCHS2; DCK; DCLKI; DCLK2; DCLK3; DCLREIA; DCLREIC; DCN;

DCPIA; DCPIB; DCPS; DCSTI; DCST2; DCSTAMP; DCTD; DCT; DCTNI; DCTN2; DCTN3; DCTN4; DCTN5; DCTNB; DCUNIDI OCX; DCXR;

DDAHI; DDAH2; DDBI DDB2; DDC; DDHDI; DDHD2; DON: DDIAS; DDIT3; DDIT4; DDIT4L; DON; ODD: DDRI; DDR2; DDRGKI; DDT; DDTL;

DDXIO; DDXII; DDXI7; DDXI8; DDXIBA; DDXI; DDX2I; DDX25; DDX27; DDX28; DDX31; DDX39A; DDX39B; DDX3X; DDX3Y; DDX4I; DDX42; DDX43; DDX4G; DDX4; DDX5D; DDX5I; DDX52; DDX53; DDX54; DDX5B; 00X58; DDX59; DOX5; DDXBD; DOX6; DEAR; DECI: DECRI; DEDD; DEFG; DEFAIB; DEFA3; DEFA4; DEFA5; DEFAG; DEFB103B; DEFBIO4B; DEFBI05A; DEFBIOBA; DEFBI08B; DEFBII2; DEFBI25;

DEFBI2G; DEFBI; DEFB4A; DEBSI; DEGS2; DEK: DENNDIA; DENNDIB; DENND2A; DENND4A; DENR; DEPDCIB; DEPDC5; DEPTDR; DERA;

OERLI; DERL2; DERL3; DES; DESI2; DEXI; DFFB; DFNA5; DFNB3I: OFNB59; DGATI; DGAT2; DGCR2; DGCRBL; DGCR8; DGKA; DGKB;

DGKD; DGKE; DGKG; DGKH; DGKI; DGKK; DGKfl; DGKZ; DGUOK; DHCR7; DHDH; DHFR; DHFRLI; DHH; DHDDH; DHPS; DHRSII; DHRS2;

DHRS4; DHRS7C; DHRS9; DHTKDI; DHXI5; DHXIB; DHX32; DHX34; DHX3B; DHX38; DHX4D; DHX58; DHX8; DHX9; DIABLO; DIAPHI; DIAPH2; DIAPH3; DICERI; DIDOI; DIEXF; DIP2A; DIP2B; DIP2C; DIRASI; DIRAS2; DIRCI; DIRC2; DIS3; DIS3L2; DIS3L; DISCI; DISPI OIXOCI; DKCI DKKI: DKK2; DKK3; DKK4; DKKLI; DLAT; DLCI; DLO; DLEC1; DLEU7; DLGI; DLB2; DLG3; DLG4; DLG5; DLGAPI; DLGAP2;

DLGAP3; DLGAP5; DLKI; DLL1; DLLS; DLL4; DLST; DLXI: DLX2; DLX3; DLX4; DLX5; OLXG; DMBTI; DMCI; DMD; DMGDH; DMKN; DMPI:

DMPK; DM TI: DMRT2; DMRT3; DMRTAI; DMTFf; DMTN; DMWD; DMXLI: DMXL2; DNA2; ONAAFI; DNAAF2; DNAAF3; DNAAF5; DNAHII;

DNAHI2; DNAHI7; DNAHI; DNAH2; DNAH3; DNAH5; ONAHB; DNAH7; DNAH8; DNAH9; DNAII; DNAI2; DNAJAI DNAJA2; DNAJA3; DNAJBM: DNAJBI3; DNAJBI; DNAJB2; DNAJB5; DNAJBB; DNAJB7; DNAJB8; DNAJB9; DNAJCIO; DNAJC12; DNAJGI3; DNAJCI4; DNAJCI5;

DNAJC18; DNAJCI9; ONAJCI; DNAJC27; DNAJC28; DNAJC2; DNAJC3; DNAJC5; DNAJC7; DNALI; DNASEI; DNASEIL2; DNASEIL3; DNASE2B; DNASE2; DNDI; DNER; DNHDI; DNLZ; DNMI: DNMIL; DNM2; DNM3; DNMBP; DNMTI; DNMT3A; DNMT3B; DNMT3L; DNPEP;

DNTT; DDC2A; DDC2B; DOCKIO; DDCKII DDCKI; DDCK2; DOCKS; D0CK4; DDCK5; DOCKS; DDCK7; DDCK8; DDCKFJ; DDHH; DDKI; DOK2;

DDK3; DOK4; DDKS; DDKB; DDK7; DDLK; DDNSDN; DDPEY2; DDTIL; DPABTI: DPCD; DPCRI: DPEPI; DPEP2; DPEP3; DPF1; DPF3; DPHI; DPH3; DPHB; DPH7; DPMI; DPM2; DPM3; DPPID; DPP3; DPP4; DPPG; DPP7; DPP8; DPPB; DPPA2; DPPA3; DPPA4; DPT; DPYI9L2;

DPYI9L3; DPY30; DPYS; DPYSL2; DPYSL3; DPYSL4; DPYSL5; DRAMI; DRAPI; ORCI; DRDI; DRD2; DRD3; DRD4; DR05; DRGI; DRGX;

DRDSHA: DRP2; OSCI; DSC2; DSC3; DSCAM; DSCAMLI; DSCCI: DSCR4; DSE; DSEL; DSGI; DSG2; DSG3; DSG4; DSP; DSPP; DST; DSTN;

DSTYK; DTDI; DTHDI DTL; DTNA; DTNB; DTNBPI; DTXI DTX2; DTX3; DTX4; DTYMK; DUOXI; DUOX2; OUDXAI; DOOXA2; DUPDI; DUS2;

DUSPIO; DUSPII; DUSPI2; DUSPI3; DUSPI4; DUSPI5; DOSPIB; DUSPI8; DUSPI; DUSP2I; DUSP22; DUSP23; DUSP27; DUSP28; DUSP2; DUSP3; DUSP4; DUSP5; DOSPB; DUSP7; DOSP8; DUSP9; OUT; DUX4; DVL2; DVL3; DXO; DYM; DYNAP; DYNCIHI; DYNC1I1: DYNCII2;

DYNCILII; DYNCZHI: DYNLLI; DYNLL2; DYNLRBI; DYNLTI; DYNLT3; DYRKIA; DYRKIB; DYRK2; DYRK3; DYSF; DYXICI: DZIPI; EZFI; E2F3;

E2F4; E2F5; E2FB; E2F7; E2F8; E4FI; EAR; EAF2; EARS2; EBAG9; EBFI: EBF2; EBF3; EBF4; EBI3; EBP; EBPL; ECD; ECE2; ECELI; ECHDCl:

ECHDC3; ECHSI; ECU; ECI2; ECSCR; ECSIT; ECT2; EDA2R; EDA; EDARADD; EDAR; EDC4; EDEMI; EDIL3; EDNI; EDN3; EDNRA; EDNRB;

EEAI; EED; EEFIAI; EEFIA2; EEFIB2; EEFID; EEFIEI; EEFIG; EEF2; EEFSEC; EFCABII; EFCABI; EFEMPI; EFEMP2; EFHB; EFHCI; EFHC2; EFHOI; EFNAI; EFNA2; EFNA3; EFNA4; EFNA5; EFNBI; EFNB2; EFNB3; EFR3A; EFR3B; EFS; EFTUDI; EFTUD2; EGF; EGFLB; EGFL7; EGFL8; EGFLAM;

EGFR; EGLNI; EGLN2; EGLN3; EGRI; EGR2; EGR3; EGR4; EHBPI: EHDI; EHD2; EHD3; EHD4; EHF; EHHADH; EHMTI; EHMT2; E124; EIDI

EIFIAD; EIFIAX; EIFIAY; EIFI; EIF2A; EIF2AKI: EIF2AK2; EIF2AK3; EIF2AK4; EIFZBI: EIF2B2; EIF2B3; EIF2B4; EIF2B5; EIFZSI; EIF2S2;

EIF2S3; EIF3A; EIF3B; EIF3C; EIF3E; EIF3F; EIF3H; EIF3J; EIF3K; EIF3M; EIF4AI; EIF4A2; EIF4A3; EIF4B; EIF4E2; EIF4E3; EIF4EBPI;

EIF4EBP2; EIF4EBP3; EIF4E; EIF4ENIFI: EIF4GI; EIF4G2; EIF4H; EIF5A2; EIF5A; EIF5; EIFB; ELACI; ELAC2; ELANE; ELAVLI; ELAVL2; ELAVL3; ELAVL4; ELFI; ELF2; ELF3; ELF4; ELF5; ELKI; ELK3; ELK4; ELL2; ELL; EL DI; ELMO2; ELMODI; ELMOD2; ELMOD3; ELSANI; ELN;

ELOFI; ELOVL2; ELOVL4; ELOVL5; ELOVLB; ELOVL7; ELP2; ELP3; ELP4; ELPB; ELSPBPI; EMB; EMCIO; EMC2; EMC3; EMC7; EMC8; EMCN;

EMD; EMEI; EMGI; EMILINI; EMILIN2; EMILIN3; EMU; EML2; EML4; EML5; EMLB; EMPI: EMP2; EMXI; EMX2; ENI; EN2; ENAH: ENAM; ENCI;

ENDDG; ENDDU; ENDDV; ENGASE; ENG; ENHO; ENO2; ENO3; ENO4; ENBPHI: ENOSFI: ENDXI: ENOX2; ENPEP; ENPPI; ENPP2; ENPP3;

ENPP5; ENPP7; ENTHD2; ENTPDI; ENTPD2; ENTPD4; ENTPD5; ENTPDB; ENTPD7; EOGT; EOMES; EP300; EP400; EPASI; EPB4ILI: EPB4IL2; EPB4IL3; EPB4IL4A; EPB4IL4B; EPB42; EPCI: EPC2; EPCAM; EPDRI; EPG5; EPGN; EPHAI: EPHA3; EPHA4; EPHA5; EPHAB;

EPHA7; EPHBI; EPHB3; EPHB4; EPHBB; EPHXI; EPHX2; EPHX3; EPM2A; EPM2AIPI; EPNI; EPO; EPOR; EPPIN; EPPIN-WFDCB; EPP I:

EPRS; EPSI5LI; EPS8; EPS8L2; EPSTi1: EPX; EPYC; ERALI; ERAPI; ERAP2; ERAS; ERBB2; ERBB2IP; ERBB3; ERBB4; ERCI; ERC2; ERCCI;

ERCC2; ERCC3; ERCC4; ERCC5; ERCCB; ERCCBL2; ERCCB-PGBD3; ERCC8; EREG; ERF; ERG; ERGICI; ERGIC2; ERGIC3; ERI3; ERICH5;

ERICHBB; ERLECI; ERLINI; ERLIN2; ERMAP; ERMARD; ERMPI; ERNI; ERN2; EROILB; ERDIL; ERP29; ERP44; ERV3-I: ERVW-I; ESAM; ESCOI; ESC02; ESD; ESFI; ESMI; ESPLI; ESPN; ESRI; ESR2; ESRPI; ESRP2; ESRRA; ESRRB; ESRRG; ESXI; ESYTI: ESYT2; ESYT3; ETAAI; ETFI; ETFA; ETFB; ETFDH; ETHEI; ETN I: ETNK2; ETNPPL; ETSI: ETS2; ETVI: ETV3; ETV4; ETV5; ETVB; ETV7; EVAIA; EVAIC; EVC2; EVC;

### EVI2A; EVI2B; EVI5; EVL; EVPL; EVX1; EVX2; EWSRI: EXO2; EXO3; EXOI; EXOCI; EXOC2; EXOC3LI; EXOC3L2; EXOC4; EXOC5; EXOC7;

EXOG; EXBSCI; EXDSC2; EXBSC3; EXBSC4; EXDSC5; EXDSCB: EXBSC7; EXDSC8; EXPH5; EXTI; EXT2; EXTL2; EXTL3; EYAI; EYA2; EYA4;

EYS; EZHI; EZH2; EZR; FID; Fll; FUR; FI2; FI3AI; FI3B; F2; F2R; F2RLI; F2RL2; F2RL3; F5; F7; F8; FB; FA2H; FAAH2; FABPI FABP2; FABP4; 5 FABP5; FABPB; FABP7; FABPB; FADD; FADSI FADS2; FADS3; FAF2; FAHD2A; FAH; FAIM2; FAIM3; FAMID2A; FAMI03AI; FAMID5A;

FAMID7A; FAMI07B; FAMIO9A; FAMIIBA; FAMIIBB; FAMIIBC; FAMIItB; FAMII4AI: FAMI20B; FAMI2DC; FAMI24B; FAMI2BA; FAMI29A;

FAMI29B; FAMI34B; FAMI35A; FAMI35B; FAM13BA; FAMI3A; FAMI3C; FAMI49A; FAMI50B; FAMI55A; FAMIBOBI; FAMI6IA; FAMIB3A;

FAMIB3B; FAMIB7A; FAMIB8A; FAMIB8B; FAMIB9B; FAMI7DA; FAMI72A; FAMI73B; FAMI74A; FAMI75A; FAMI75B; FAMI77AI: FAMI77B;

FAMI78A; FAMI78B; FAMI79B; FAMI8DA; FAMI84A; FAMI84B; FAMI87B; FAMI88A; FAMI88B; FAMI89B; FAMI93B; FAMIBBA; FAMI9BB; ID FAMI98B; FAMI9AI: FAMI9A2; FAMI9A4; FAMI9A5; FAM2D4A; FAM2D5A; FAM2DA; FAM2DB; FAM20C; FAM2IDB: FAM2I3A; FAM2I4A;

FAM2IBA; FAM220A; FAM227B; FAM32A; FA 3A: FAM3B; FAM3C; FAM3D; FAM4BA; FAM4BD; FAM49A; FAM49B; FAM5BB; FAM53B;

FAM57A; FAM58A; FAMBDA; FAMB3B; FAMB4A; FAM65B; FAMBBC; FAM7IFI; FAM7IF2; FAM72B; FAM78B; FAM8IB; FAM83A; FAM83B;

FAM83D; FAM83H; FAM84A; FAM84B; FAM89A; FAM92AI; FAM92B; FAM9B; FAN1; FANCA; FANCB; FANCC; FANGD2; FANCE; FANCF; FANCG; FANCI; FANCL; FANCM; FANKI; FAP; FARI; FARPI; FARP2; FARS2; FARSA; FAS; FASLG; FASN; FASTKD2; FASTK; FATI; FAT2; FATS; 15 FAT4; FATEI; FALL FBFI; FBL; FBLIMI; FBLNI; FBLN2; FBLN5; FBNI; FBN2; FBN3; FBPI; FBP2; FBRS; FBXLI5: FBXLI7; FBXLI9; FBXL2D;

FBXL2; FBXL3; FBXL4; FBXL5; FBXL7; FBXOID; FBXDII; FBX0I5; FBXDI7; FBXDI8; FBXD28; FBXD3D; FBXD3I; FBXD32; FBXD33; FBXD38;

FBXD3; FBXD4D; FBX047; FBXD4; FBX05; FBXD7; FBXD8; FBXD9; FBXWIL FBXW4; FBXW7; FBXW8; FCAR; FCERIA; FCERIG; FCER2;

FCGBP; FCGR2A; FCGR2B; FCGR3A; FCGR3B; FCGRT; FCHOI; FCHD2; FCHSD2; FCN FCN2; FCRLI; FCRL2; FCRL3; FCRL4; FCRL5;

FCRLB; FCRLA; FCRLB; FDFTI; FDPS; FDXI; FDXIL; FDXR; FECH; FEMIA; FEMIB; FEMIC; FENI; FERD3L; FER; FERMTI; FERMT2; FERMT3; 0 FES; FEV; FEZI; FEZ2; FEZFI; FEZF2; FFARI; FFAR2; FFAR3; FFAR4; FGA; FGB; FGDI; FGD2; FGD3; FGD4; FGD5; FGDB; FGFID; FBFII; FGFI2;

FGFI3; FGFI4; FGFIB; FGFI7; FGFI8; FGFI9; FBFI; FGF2D; FGF2I: FGF23; FGF2; FGF3; FGF4; FGF5; FGFB; FGF7; FGF8; FGF9; FGFBPI;

FGFBP2; FGFBP3; FGFRI; FGFRIDP2; FGFRIDP; FGFR2; FGFR3; FGFR4; FGFRLI; FGG; FGGY; FGLI; FGL2; FGR; FHDCI FH; FHIT; FHLL FHL2;

FHL5; FHDDI; FHDD3; FIBP; FIG4; FIGF; FIGLA; FIGN; FILIPI; FILIPIL; FIP1L1; FISL FIT I; FITM2; FJXI; FKBPID; FKBPII; FKBPI4; FKBPI5;

FKBPIA; FKBPIB; FK8P2; FKBP3; FKBP4; FKBP5; FKBPB; FKBP7; FKBP8; FKBP9; FKBPL; FKRP; FKTN; FLADI FLCN; FLG2; FLG; FLU; FLU; 5 FLNA; FLNB; FLNC; FLOTI; FLDT2; FLRT2; FLRT3; FLTI: FLT3; FLT3LG; FLT4; FLVCRI; FLVCR2; FMNL FMN2; FMNLI; FNL2; FMNL3; FHOI:

FMD2; FMD3; FMD4; FMOD; FMRI; FNI; FN3K; FN3KRP; FNBPI; FNDCI; FNDC3A; FNDC3B; FNDC4; FNTA; FNTB; FDCAD; FOLHI FDLR1;

FDLR2; FDLR3; FOPNL; FOSB; FDS; FDSLI FDSL2; FOXA1; FOXA2; FOXA3; FOXBI; FOXCI FOXC2; FOXDI; FOXEI; FOXFI; FOXF2; FOXGI;

FOXHI; FOXII; FDXJI; FDXJ2; FOXKI; FOXK2; FOXLI; FOXL2; FOXMI; FOXNI; FOXN2; FOXN3; FOXOl; FOXO3; FOXO4; FOXOB; FOXPI FOXP2;

FOXP3; FOXP4; FOXQI; FOXRI; FOXR2; FOXREDI; FPGS; FPRI; FPR2; FPR3; FRASI; FRATI; FRAT2; FREMf FREM2; FREM3; FRGI; FRG2; 0 FRK; FRMD3; FRMD4A; FRMD4B; FRMD5; FRMDB; FRMD7; FRMPDI; FRMPD2; FRMPD4; FRRSIL; FRS2; FRS3; FRY; FRYL; FRZB; FSBP;

FSCB; FSCNI; FSCN2; FSDI; FSDIL; FSD2; FSHB; FSHR; FSIPI; FST; FSTLI; FSTL3; FSTL4; FSTL5; F D; FTHI FTL; FTMT; FTO; FTSJI;

FTSJ2; FTSJ3; FUBPI: FUBP3; FUCAt: FOCA2; FUNDCI: FONDC2; FDRIN; FUS; FOTIO; FOTII; FUTI; FDT2; FUT3; FUT4; FUT5; FUTG; FUT7;

FUT8; FOT9; FUZ; FXN; FXRI; FXR2; FXYDI; FXYD2; FXYD3; FXYD5; FXYDB; FYS; FYCDI; FYN; FZDIO; FZDI; FZD2; FZD3; FZD4; FZD5; FZDB;

FZD7; FZDB; FZD9; FZRI; GDS2; G2E3; G3BPI; GGPC2; GGPC3; GBPC; GBPD; GAA; GABI; GAB2; GAB3; GABARAP; GABARAPLI; 5 GABARAPL2; GABBRI: GABBR2; GABPA; GABPBI; GABRAI; GABRA2; GABRA3; GABRA4; GABRA5; GABRAB; GABRBI; GABRB2; GABRB3; GABRE; GABRGI; GABRG2; GABRG3; GABRP; GABRQ; GABRRI; GABRR2; GADI; GAO2; GADD45B; GAOD45G; GADD 5GIPI; GADLI;

GAGEID; GAGEI; GAK; GALC; GAL; GALKI; GALK2; GALN; GALNS; GALNTI2; GALNTI3; GALNTI4; GALNTI5; GALNTI8; GALNTI; GALNT2;

GALNT3; GALNT4; GALNT5; GALNTB; GALNT7; GALNT8; GALNT9; GALNTLB; GALP; GALRI; GALR2; GALR3; GALT; GAMT; BANAB: GANG;

GAN; GAP43; GAPDH; GAPDHS; GARI; GAREM; GARNL3; GARS; GART; GASI; GAS2; GAS2LI; GASB; GAS7; GAS8; GAST; GATAI GATA2; GATA3; GATA4; GATA5; GATAB; GATADI GATAO2A; GATAD2B; GATB; GATM; GBA2; GBA; GBAS; GBEI; GBFI; GBGTI; BBXf; GBX2; GCA;

GCCI; GCDH; GCFC2; GCG; GCGR; GCHI; GCHFR; GC; GCK; GCKR; GCLC; GCMI; GCM2; GCNILI; GCNTI; GCNT2; GCNT3; GCNT7; GCOHI;

GCSAM; GCSAML; GCSH; GDA; GDAPI; GOEI; GDFIQ; GDFII; GDFI5; GDFI: GDF2; GDF3; GDF5; GDFB; GDF7; GDF9; GDII; GDI2; GDNF;

GDPD3; GDPO5; GEM; GEMIN2; GEMIN4; GE IN6; GENI GET4; GFAP; GFER; GFIIB; GFII; GFMI; GFPTI; GFPT2; GFRAI; GFRA2; GFRA3; GFRA4; GGAI; GGA3; GGACT; GGCT: GGCX; GGH; GGNBP2: GGN; GGPSI; GGTI; GGT2; GGT5; GGTLCI; GHI; GH2; BHITM; GHRH; GHR; GHRHR; GHRL; GHSR; GID8; GIF; GIGYFI; GIGYF2; GIMAP5; GIMAP7; GIMAP8; GINI; GINSI; GINS2; GIPCI; GIPC3; GIP; GIPR; GITI; GIT2;

GJAI; GJA3; GJA8; GJBI; GJB2; GJBB; GJCI; GJC2; GJC3; GJD2; GJB3; GK; GKNI: GKN2; GLA; GLBI; GLCCII; GLCE; GLDC; GLDN; GLEI;

GLGI; GUI: GLI2; GLI3; GLIPRI; GLIPR2; GLIS2; BLISS; GLMN; GLDI: GLDD4; GLPIR; GLP2R; GLRAI; GLRA2; GLRA3; GLRB; GLRX2; GLRX3;

GLRX5; GLRX; GLS2; GLS; GLTIDI; GLTBDI; GLT8DI; GLTSCRI; GLTSCR2; GLUDI: GLUD2; GLUL; GLYAT; GLYATL3; GLYCTK; GM2A; GMCLI;

GMDS; GMFB; GMFG; GMIP; GML; GMNN: GMPPA; GMPPB; GMPR2; GMPR; GMPS; GNAII; GNAI2; GNAI3; GNAI4; GNAI5; GNAII; GNAI2; GNAL; GNADI; GNAQ: GNAS; GMATI: GNATS; GNAZ; GNBIL; GNB2LI; GNB3; GNB4; GNB5; GNE; GNGIB; GNGII; GNG2; GNG4; GNG7; GNG8;

GNGTI; GNGT2; GNU; GNL3; GNL3L; GNLY; GNMT; GNPAT; GNPDAI; GNPDA2; GNPTAB; GNPTG; GNRHI; GNRH2; GNRHR; GNS; GOLGAI;

GDLGA2; GOLGA3; GDLGA4; GDLGA5; GDLGA8B; GDLGBI; GDLMI; GBLPH3; GBLTIA; GBLTIB; GBN4L; GDPC; GDRAB; GDRASPI;

GDRASP2; GDSRI; GOSR2; GDTI: GDT2; GPIBA; GPIBB; GP2; GP5; BPS; GP9; GPA33; GPAAI; GPALPPI; GPAM; GPANKI; GPAT2;

GPATCHI; GPATCH2; GPATCH2L; GPATCH8; GPBARI; GPCI; GPC2; GPC3; GPC4; GPC5; GPCB; GPCPDI; GPDI; GPDIL; GPD2; GPERI; GPHA2; GPHN; GPIHBPI; GPI; GPKOW; GPLDI; GPMBA; GPMBB; GPNI; GPNMB; GPRIDI: GPRIIB; GPRI2; GPRI32; GPRI35; GPRI37C;

GPRI39; GPR143; GPRI48; GPRI49; GPRI50; GPRI5I; GPRI52; GPR153; GPRI55; GPRI5B; GPRI58; GPRI5; GPRIBO; GPRIGI; GPRIB2;

GPRI7I; GPRI74; GPRI7B; GPRI7B; GPRI7; GPRI80; GPRI82; GPRI83; GPRI9; GPRI: GPR2D; GPR22; GPR2B; GPR34; GPR35; GPR37;

GPR37LI; GPR39; GPR4; GPR5D; GPR52; GPR55; GPRB5; GPRB8; GPRB; GPR75; GPR78; GPR83; GPR87; GPRASPI; GPRC5A; GPRC5B;

GPRC5C; GPRC5D; GPRCBA; GPRINI: GPRIN2; GPRIN3; GPS2; GPSHI: GPSM2; GPSM3; GPT2; GPT; GPX5; GRAMOIB; GRAMB3; GRAMD4; GRAP2; GRAP; GRASP; GRBID; GRBI4; GRB2; GRB7; GREBI; GREMI; GREM2; GRHLI; GRHL2; GRHPR; GRIAI; GRIA2; GRIA3;

GRIA4; GRIDI; GRID2; GRIKI; GRIK2; GRIK4; GRIK5; GRINI: GRIN2A; GRIN2B; GRIN2C; GRIN2D; GRIN3A; GRIN3B; GRINA; GRIPI; GRKI;

GRK4; GRK5; GRKB; GRK7; GRMI; GRM2; GRM3; GRM4; GRM5; GRMB; GRM7; GRMB; GRN; GRPELI; GRP; GRPR; GRSFI; GRXCRI; GRXCR2;

GSC2; GSC; GSDMA; GSDMB; GSDMC; GSDMD; GSEI; GSGIL; GSK3A; GSK3B; GSN; GSPTI; GSPT2; GSR: GSS; GSTAI; GSTA2; GSTA4;

GSTA5; GSTCD; GSTI GSTM3; GSTDI: GSTD2; GSTPI; GSTTI: GSTT2B; GSTT2; GSTZI; GSXI: GSX2; GTDCI GTF2AI; GTF2AIL; GTF2A2; GTF2EI; GTF2E2; GTF2FI; GTF2F2; GTFZHI GTF2H2C2; GTF2H2C; GTF2H2; GTF2H3; GTF2H4; GTF2H5; GTF2I; GTF2IRDI; GTF2IRD2;

GTF3A; GTF3CI; GTPBPI: GTPBP3; GTP8P4; GTSFI: GDCA1A; GUCAIB; GUCDI; GUCYIA2; GDCYIA3; GDCYIB3; GUCY2C; GUGY2D;

GUCY2F; GDKI; GULPI; GDSB; GYGI; GYG2; GYLTLIB; GYPA; GYPB; GYPC; GYPE; GYSI; GYS2; GZFI: GZMA; GZMB; GZMH; GZMK; GZMM;

HIFD; HIFX; H2AFJ; H2AFX; H2AFY2; H2AFY; H2AFZ; H2BFWT; H3F3B; HAAO; HABP2; HABP4; HACDI HAC02; HACD4; HACEI; HACLI;

HADHA; HADHB; HADH; HAGH; HAL; HAMP; HANOI; HAND2; HADI: HA02; HAPI; HAPLNI: HAPLN4; HARS2; HARS; HASf HAS2; HAS3; HATI; HAVCRI; HAVCR2; HAXI; HBA2; HBB; HBO; HBEI: HBEGF; HBGI: HBG2; HBM; HBPI; HBQI HBSIL; HBZ; HCARI; HCAR2; HCAR3; HCCS; HCFCI; HCFCZ; HCK; HCLSI; HCNI; HCN2; HCN3; HCN4; HCRT; HCRTRZ; HCST; HDACIO; HDACIt HDACI; HDACZ; HDAC3; HDAC4;

HDAC5; HDACB; HDAC7; HDAC8; HDAC9; HDC; HDDCZ; HDGF; HDBFLI; HDGF P3; HDHDI; HDLBP; HEAT I; HEATR3; HEATR5B; HEATRB;

HEBPI; HEBPZ; HECA; HECTDZ; HECTD4; HECWI: HECWZ; HEGI; HELLS; HELQ; HELT; HELZZ; HELZ; HEMGN; HEPACAM; HEPH; HEPNI;

HERCI; HERC2; HERC3; HERC5; HERCB; HERPUOI; HESI; HES2; HESB; HES7; HESXI; HEXA; HEXB; HEXDC; HEXIMI; HEYI; HEYZ; HFE; 5 HFMI; HGD; HGFAC; HGF; HGHI; HGS; HGSNAT; HHAT; HHATL; HHEX; HHIP; HHIPLI; HHLAI; HHLAZ; HIATI; HIBAOH: HIBGH; HICI; HICZ;

HIDI; HIFIA; HIFIAN; HIF3A; HIGDIA; HIGDIC; HIGDZA; HILPDA; HINFP; HINTI; H1NT2; HIPI; HIPIR; HIPKI; HIPKZ; HIPK3; HIRA; HIRIP3;

HISTIHIA; HISTIHIB; HISTIHIC; HISTIHID; HISTIHIE; HISTIHIT; HISTIHZAE; HISTIH2AH; HISTIHZBG; HISTIH2BH; HISTIH2BM; HISTIH3G;

HISTIH4D; HIST3H3; HIVEPI; HIVEP2; HJURP; HKI; HK2; HK3; HKDCI; HLA-A; HLA-B; HLA-C; H LA-DMA; HLA-DMB; HLA-DOA; H LA-DOB;

HLA-DPAI; HLA-DPBI; HLA-DQAI; HLA-DQA2; HLA-OQBI: HLA-DOB2; HLA-DRA; HLA-DRBI; HLA-DRB3; HLA-DRB4; HLA-DRB5; HLA-E; ID HLA-F; HLA-G; HLCS; HLF; HLTF; HLX; HMI3; HMBOXI; HMBS; HMCNI; HMG2DA; HMG20B; HMGAI: HMGA2; HMGBI; HMGBZ; HMGB3;

HMGCR; HMGCSI; HMGNI; HMGN4; HMGN5; HMGXB3; HMGXB4; HMHAI; HMMR; HMOXI; HMDXZ; HMPIB; HMSD; HMXI; HNI; HNIL; HNFIA;

HNFIB; HNF4A; HNF4G; HNT: HNRNPAO; HNRNPAI: HNRNPAZBI; HNRNPA3; HNRNPAB; HNRNPC; HNRNPD; HNRNPDL; HNRNPF;

HNRNPHI; HNRNPHZ; HNRNPK; HNRNPL; HNRNPM; HNRNPR; HNRNPU; HNRNPULI: HDGAI; HOMER): HOMERZ; HOMERS: HOMEZ;

HODKZ; HODK3; HOPX; HORMAD2: HOXAIO; HOXAII; HOXAI3; HOXAI; HOXA2; HOXA3; HOXA4; HOXA5; HOXAB; HOXA7; HOXA9; HOXBI3; HOXBI; HOXB2; HOXB3; HOXB4; HOXB5; HOXB6; HOXB7; HOXB8; HOXB9; HOXC10; HOXCII; HOXCI2; HOXCI3; HOXC4; HOXC5; HOXCB;

HOXC8; HOXCB; HOXDIO; HOXDII; HOXDI2; HOXDI3; HOXDI: HOXD3; HOXO4; HOXO8; HOXO9; HPIBP3; HPCALI; HPD; HPGD; HPGDS; HP;

HPN; HPR; HPRTI; HPSI; HPS3; HPS4; HPS5; HPSB; HPSE2; HPSE; HPX; HRAS; HRASLS; HRC; HRG; HRHI; HRH2; HRH3; HRH4; HR;

HRK; HRNR; HRSPIZ; HSIBP3; HS3STI; HS3STZ; HS3ST3AI; HS3ST3BI; HS3ST4; HS3ST5; HS3STB; HSBSTI; HSBSTZ; HSBST3; HSBPI;

HSDIIBI; HSOIIBIL; HSDIIB2; HSDI7BIO; HSDI7BII; HSDI7BI2; HSDI7BI3; HSDI7BI4; HSDI7BI; HSDI7B2; HSDI7B3; HSDI7B4; HSDI7B6; HSDI7B7; HSDI7BB; HSD3BI; HSD3B7; HSDLI; HSDL2; HSFI; HSFZ; HSF4; HSF5; HSFY2; HSH2D; HSP90AAI: HSP90ABI; HSP90BI;

HSPAI2A; HSPAI2B; HSPAI3; HSPAI4; HSPAIA; HSPAIL; HSPA2; HSPA4; HSPA4L; HSPA5; HSPAB; HSPA8; HSPA9; HSPBI: HSPB2;

HSPB3; HSPBB; HSPB7; HSPB8; HSPB9; HSPBAPI; HSPBPI HSPDI; HSPEI: HSPG2; HSPHI; HTATIPZ; HTNI; HTN3; HTRIA; HTRIB;

HTRIE; HTRIF; HTRZA; HTRZB; HTRZC; HTR3A; HTR3B; HTR3C; HTR3D; HTR3E; HTR4; HTR5A; HTR7; HTRAI; HTRAZ; HTRA3; HTRA4;

HTT; HUNK; HUSIB; HUSI; HUWEI; HVCNI; HYALI; HYALZ; HYAL3; HYAL4; HYDIN; HYKK; HYLSI; HYOUI: HYPM; IAPP; IARS2; IARS; IBA57; 5 IBSP; IBTK; ICAI; ICAMI; ICAMZ; ICAM3; ICAM4; ICAM5; ICEI ICE2; ICK; ICMT; ICOS; ICOSLG; IDI ID2; 104; IDE; IDHI; IDH2; IDH3B; IDNK;

IDOI; ID02; IDS; IDUA; IER2; IER3; IER3IPI; IER5; IFFOI: IFFOZ; IFIIB; IFIZ7; IF130: IF135: IF144; IFIHI; IFITI; IFITZ; IFIT3; IFIT5: IFITMI;

IFITMZ; IFITM3; IFITM5; IFNAIO; IFNAI3; IFNAI4; IFNAIB; IFNAI7; IFNAI; IFNAZI; IFNA2; IFNA4; IFNA5; IFNAG; IFNA7; IFNA8; IFNARI:

IFNAR2; IFNBI; IFNE; IFNG; IFNGRI; IFNGR2; IFNK; IFNLI: IFNL2; IFNL3; IFNWI; IFRDI; IFTI22; IFTI40; IFTI72; IFT27; IFT43; IFT57; IFT74;

IFT80; IFT88; IGBPI; IGDCC3; IGFI; IGFIR; IGF2BPI; IGF2BP2; IGF2BP3; IGF2; IGF2R; IGFALS; IGFBPI; IGFBP2; IGFBP3; IGFBP4; IGFBP5; 0 IGFBPB; IGFBP7; IGFBPLI; IGFLI: IGFLRI; IGHMBP2; IGJ; IGLLI; IGSFIO; IGSFII; IGSFI; IGSF5; IGSFB; IGSF8; IHH; IKB AP; IKBKB; IKBKE;

IKBKG; IK; IKZFI; IKZF2; IKZF3; IKZF4; ILIO; ILIORA; ILIORB; ILII; ILIIRA; ILIZA: ILI2B; ILI2RBI; IL13; ILI3RAI; ILI3RA2; IL15: ILI5RA; ILIB;

ILI7A; ILI7B; ILI7C; ILI7D: ILI7F; ILI7RA; ILI7RB; ILI7RC; ILI7RD; ILI7RE; ILI7REL; ILI8BP; ILI8: ILI8RI; ILI8RAP; ILI9; ILIA; ILIB; ILIFIO;

ILIRI; ILIRZ: ILIRAP; ILIRAPLI: ILIRAPLZ; ILIRLI; ILIRLZ; ILIRN; ILZO; ILZDRA; ILZORB; ILZI; ILZIR; ILZZ; IL22RA2; ILZ3A; IL23R; IL24;

IL25; IL2B; IL27; ILZ7RA; IL2: IL2RA; IL2RB; IL2RG; IL3I; IL3IRA; IL32; IL33; IL34; IL3BA; IL3BB; IL3GG; IL3GRN; IL37; IL3: IL3RA; IL4; 5 IL4II; IL4R; IL5; IL5RA; ILB; ILBR; ILBST; IL7; IL7R; IL9: IL9R; ILDRI; ILDRZ; ILFZ; ILF3; ILK; IMMPIL; IMMPZL; IMMT; IMPS; IMPAI; IMPAZ; IMPACT; IHPAD!; IMPDHI; IMPDH2; IMPBI; IMPG2; 1NADL: INA; INCENP; INF2; INGI; ING2; ING3; ING4; ING5; 1NHA; INHBA; INHBB; INHBC; INHBE; INB8BB; INOSDD; IND8D; INPPI INPP4A; INPP4B; INPP5A; INPP5B; INPP5D; INPP5E; INPP5F; INPP5K; INPPLI; INSC: INS; INSIGI; INSIG2; INS-IGF2; INSL3; INSL4; INSLB; INSMI INSM2; INSR; INSRR; INTSI2; INTSI; INTS2; INTS3; INTS4; INTS5; !NTSB; 1NTS7; INTS8; INTU; INVS; !PBKI; IP6K2; IPGK3; IPCEFI; IPM; IPBtl; IPDI3; IPB7; IPB8; IPB9; IDCBI; IDCG; IQCH; IQCJ; IDCJ-SCHIPI IflC; IDGAPI; IBGAP2; IQGAP3: IDSECI: IQSEC2; IQSEC3; IRAKIBPI; IRAKI; IRAK2; IRAK3; IRAK4; IREB2; IRFI; IRF2BP2; IRF2BPL; IRF2; IRF3; IRF4; IRF5; IRFB; IRF7; IRF8; IRF9; IRGI IRGM; IRSI; IRS2; IRS4; IRXI; IRX2; IRX3; IRX4; IRX5; ISCU; ISG2B; ISLI; ISMI ISM2; ISPD; ISTI; ISX; ISYNAI; ITCH; ITFGI; ITFG3; ITGAIO; ITGAII; ITGAI; ITGA2B; ITGA2; ITGA3; ITGA4; ITGA5; ITGAB; ITGA7; ITGA8; ITGA9; ITGAD; ITGAE; ITGAL; ITGAM; ITGAV; ITGAX; ITGBIBPI; ITGBI; ITGB2; ITGB3; ITGB4; ITGB5; ITGBB; ITGB7; ITGB8; ITGBLI; ITIHI; ITIH2: ITIH3; ITIH4; ITIH5: ITK; ITLNI: ITLN2; ITM2A; ITM2B; ITM2C; ITPA; ITPKt; ITPKA; ITPKC; ITPRI; ITPR2; ITPR3; ITSNI; ITSN2; IVD: IVL; IVNSIABP; IYD: IZUMBI; JAOEI; JADE2; JABI JAG2; JAGNI; JAK2; JAK3; JAK IPI: JAKMIP2; JAKMIP3; JAM2; JAM3; JARID2; JAZFI JDP2; JMJOIC; JMJDB; JMY; JPH2; JPH3; JPH4; JR; JS PI; JTB; JUNB; JUNO; JUP; KALI; KALRN; KANKI; KANK2; KANK4; KANSLI: KANSL3; KARS; KAT2A; KAT2B; KAT5; KATBA; KATBB; KAT7; KAT8; KATNAI; KATNAL2; KATNBI; KAZALDI; KAZN; KBTBDII: KBTBDI3; KBTBD8; KCMFI; KCHAI: KCNA4; KCNA5; KGNAB; KCNA7; KCNABI: KCNAB2; KCNAB3; CNBI; KCNB2; KCNCI; KCNC2; KCNC3; KCND2; KCNEi KCNE2; KCNE3; KCNE4; KCNE5; KCNG3; KCNG4; CNHI; KCNH2; KCNH3; KCNH4; KCNH5; KCNHB; KCNH7; KCNH8; KCNIPI; KCNIP2; KCNIP3; KCNIP4; KCNJID; KCNJ1I; KCNJ12; KCNJI3; KCNJ14; KCNJI5; KCNJIB; KCNJI8; KCNJi; KCNJ2; KCNJ3; KCNJ4; KCNJ5; KCNJ6; KCNJB; KCNJ9; KCNKIB; KCNKI2; KCNKIB; KCNKI7; KCNKI8; CNKI; KCNK2; KCNK3; KCN 5; KGNKB; KCNK9; KCNMAI; KCN BI; KCNMB2; KCNMB3; KCNMB4; KCNNI; KCNN2; KCNN3; KCNN4; KCNOI; KCNO2; KCNO3; KCNQ5; KCNRG; KCNSI; KCNS3; KCNTI; KCNT2; CNUI; KCNVI; KCNV2; KCTDID; CTDII; KCTDI2; KCTOI3; KCTDI5; KCTDIB; KCTDI; KCTD2I; KCTD2; KCTD3; KCTDB; KCTD7; KCTD9; KDELCI: KDELRI; KDM2A; KDM2B; KDM3A; KDM3B; KDM4B; KDM4C; KDM5A; KDM5B; KDM5C; KDM5D; KDMBA; KDMBB; KDR; KOSR; KEAPI; KEL; KERA; KHDC3L; KHDRBS2; KHBRBS3; KHK; KHSRP; KIAADB2B; KIAAQD4D; KIAADIBD; KIAADIDI KIAADIBB; KIAAD22G; KIAAD22BL; KIAAD232; KIAAD3I9; KIAAD3I9L; KIAAD355; KIAAD3BI; KIAAB58G; KIAAD825; KIAAD9D7; KIAAD922; KIAAID24; KIAAI033; KIAAIIO9; KIAAII47; KIAAIIBI; KIAAI2II; KIAAI2IIL; KIAAI2I7; KIAAI279; KIAAI324; KIAAI324L; KIAAI377; KIAAI45B; KIAAI4B2; KIAAI4B8; KIAA1524; KIAA1549; KIAAI55I; KIAA1598; KIAAI7I5; KIAAI8D4; KIAAI84I; KIAAI9I9; KIAA2D22; KIDINS220; KIFII; KIFI3A; KIFI4; KIFI5: KIFIBB; KIFI7; KIFI8A; KIFIA; KIFIC; KIF20A; KIF2BB; KIF2IA; KIF2IB; KIF22; KIF23; KIF24; KIF25; KIF2BA; KIF2BB; KIF2A; KIF2B; KIF2C; KIF3A; KIF3B; KIF3C; KIF4A; KIF4B; KIF5A; KIF5B; KIF5C; KIFB; KIF7; KIF9; KIFAP3; KIFCI; KIFC3; KIN; KIR2DLI; KIR2DL2; KIR2DL3; KIR2DL4; KIR2DL5A; KIR2DL5B; KIR2DSI; KIR2DS2; KIR2DS3; KIR2DS4; KIR3DLI; KIR3DL2; KIR3DL3; KIRREL2; KIRREL3; KIRREL; KISSI; ISSi; KIT; KITLG; KLB; KLCI; KLFID; KLFII KLFI2; KLFI3; KLFI4; KLFI5; KLFIB; KLFI KLF2; KLF3; KLF4; KLF5; KLFB; KLF7; KLF8; KLF9; KLHDCI; KLHDC2; KLHDC8A; KLHDC8B; KLHLIO; KLHLI2; KLHLI; KLHL2D; KLHL25; KLHL2B; KLHL29; KLHL2; KLHL3I; KLHL35; KLHL3; KLHL40; KLHL4I; KLHL42; KLHL5; KLHLB; KLHL7; KLHLB; KL; KLKIO; KLKII; KLKI2; KLKI3; KLKI4; KLKI5; KLKI; KLK2; KLK3; KLK4; KLK5; KLKB; KLK7; KLK8; KLK9; KLKBI; KLLN; KLRBI; KLRCI; KLRC2; KLRC4; KLRC4-KLRKI: KLRDI; KLRGI; KLRG2; KMD; KMT2A; KMT2B; KMT2C; KMT2D; KMT2E; KNGI; KNSTRN; KNTC1; KPNAI; KPNA2; KPNA3; KPNA4; KPNAB; KPNA7; KPNBI; KPRP; KPTN; KRAS; KRBOX4; KREMEN1; KRITI; KRTID; KRTI2; KRTI3; KRTI4; KRTI5; KRTIB; KRTI7; KRTI8; KRTI9; KRTI; KRT2D; KRT23; KRT2; KRT3I: KRT32; KRT34; KRT35; KRT3; KRT4; KRT5; KRTBA; KRTBB; KRTGC; KRT7I; KRT72; K T74; KRT75; KRT7B; KRT78; KRT7; KRT8D; KRT8I; KRT82; KRT83; KRT85; KRT8G; KRT8; KRT9; KRTAPII-I; KRTAP5-I; KRTAP5-9; KSRI KSR2; TNI; KYNU; LICAM; L2HGDH; L3MBTLI; L3MBTL2; L3MBTL3; L3MBTL4; LACCI; LACEI; LACRT; LACTB; LADI; LAG3; LAIRI: LAIR2; LALBA; LAMAI; LAMA2; LAMA3; LAMA4; LAMA5; LAMB!: LAMB2; LAMB3; LAMB4; LA CI; LAMC2; LAMC3; LAMP1: LAMP2; LAMP3; LAMTORI; LAMTQR2; LAMTOR3; IANCLI: LANCL2; LAP3; LAPTM4B; LARGE; LARP1;

LARP4; LARPG; LARP7; LARS2; LARS; LASIL; LASPI; LAT2; LAT; LATSI; LATS2; LAXI; LAYH; LBH; LBP; LBR; LBXI LBX2; LCA5; LCAT;

LCEIC; LCE3A; LCE3B: LCE3C; LCE3D; LCE3E; LCE5A; LCK; LCLATI; LCMTI; LCNI: LCN2; LCOR; LCPt: LCP2; LCT; LDBI; LDB2; LDB3;

LDHA; LDHALBB; LDHB; LDHC; LDHD; LDLRAD3; LDLRAD4; LDLR; LDDCI; LDDCIL; LEAP2; LECT1; LECT2; LEFI; LEFTYI; LEFTY2; LEKRI; LEMD2; LEMD3; LEDI; LEP; LEPRDTLI; LETMI; LETMDI; LEUTX; LFNG; LGALSI2; LGALSI3; LGALSI4; LGALSIB; LGALSI; LGALS2; LGALS3BP;

LGALS3; LGALS4; LGALS7B; LGALS8; LGALSB; LGALSL; LGII; LGI2; LGI4; LGMN; LGR4; LGR5; LGRB; LGSN; LHB; LHCGR; LHFP; LHFPLI:

LHFPL2; LHFPL3; LHFPL4; LHFPL5; LHPP; LHXI; LHX2; LHX3; LHX4; LHX5; LHXB-. LHX9; LIAS; LIE; LIFR; LIGI; LIG3; LIG4; LILRAI; LILRA2;

LILRA3; LILRA4; LILRA5; LILRBI; LILRB2; LILRB3; LILRB4; LILRB5; LIM2; LIMAI: LIMCHI; LIMDI; LIND2; LIMKI; LI K2; LIMSI; LIMS2;

LIN28B; LIN52; LIN7A; LIN7B; LIN7C; UNO; LINGDI; LING02: LINGB4; LINS; LIPA; LIPC; LIPE; LIPF; LIPG; LIPH; LIPI; LIPN; LIPTI: LIPT2; LITAF; LIXI; LLGLI; LLGL2; LMANI; LMANIL: LMAN2L; LMBRI; LMBRDI; LMBRD2; L CDI; LMFi: LMLN; LMNA; LMNBI L NB2; LMNTDI; LMOI;

LMO2; LMO3; LMO7; LMODI; LDD3; LMTK2; LT3; LMXIA; LMXIB; LNPEP; LNXI; LNX2; LDCID02889BB; LOCIOIOBD32I;

LDCID2723475; LOCI0272399B; LOCI02724127; LDC1027245BD; LDC102724770; LQC1027250IB; LOCID2725035; LDC400499;

LOC400927-CSNKIE; LOCB45I77; LONPI; LONRFI LOR; LOXHDI: LOX; LOXLI; LOXL2; LOXL3; LOXL4; LPA; LPARI; LPAR2; LPAR3; LPARB;

LPCATI; LPCAT2; LPCAT3; LPCAT4; LPGATI; LPINI; LPIN2; LPL; LPD; LPP; LPPR2; LPPR4; LPXN; LRAT; LRBA; LRCHI; LRCH4; LRFN2; LRFN5; LRGI; LRGUK; LRIFI; LRIGI; LRIG3; LRITI; LRIT3; LRMP; LRPID; LRPI2; LRPIB; LRPI; LRP2BP; LRP2; LRP4; LRP5; LRPB; LRPAPI;

LRPPRC; LRRI; LRRCI5; LRRCIBA; LRRCIBB; LRRCI7; LRRCI8; LRRCI; LRRC2D; LRRC2B; LRRC3D; LRRC32; LRRC37A; LRRC37B;

LRRC39; LRRC49; LRRC4B; LRRC4C; LRRC4; LRRC52; LRRC59; LRRCBI; LRRCB3; LRRCB9; LRRCB; LRRC74A; LRRC7; LRRC8A;

LRRC8C; LRRCCI; LRRFIPI; LRRFIP2; LRR I; LRRK2; LRRNI; LRRN2; LRRN3; LRRN4; LRRTMI; LRRTM2; LRRTM3; LRRTM4; LRSAMI;

LRTMI; LRTDMT; LSAMP; LSMII: LSMI: LSM2; LSM3; LSM4; LS 5; LSMB; LSM7; LSPI; LSR; LSS: LSTI; LTA4H; LTA; LTB4R2; LTB4R: LTB; LTBPI; LTBP2; LTBP3; LTBP4; LTBR; LTC4S; LTF; LTK; LOC7L3; LOC7L; LUM; LURAPIL; LUZP4; LOZPB; LVRN; LXN; LYBD; LYBE; LYBG5B;

LYBG5C; LYBGBC; LYBGBF; LYB; LY75-CD302; LY75; LY8B; LY9B; LY9; LYL1; LYN; LYNX; LYPDI; LYPD2; LYPD3; LYPD4; LYPD5;

LYPOB; LYPD8; LYPLAI; LYPLALI; LYRM1: LYR 4; LYRM7; LYRM9; LYST; LYVEI; LYZ; LYZLI; LYZL2; LYZLB; LZTFLI: LZTRI: LZTSI; MBPR;

MAATSI; MAB2ILI; MAB2IL2; MAB2IL3; MACCI; MACRDDI; MACROD2; MAOILI; MAD2LIBP; MAO2LI; MAD2L2; MADCAMI; MAOD; MAEA:

MAEL; MAPI; MAFA; MAFB; MAFF; MAFG; MAF; MAFK; MAGEAIO; MAGEAII; MAGEAI2; MAGEAI; MAGEA2B; MAGEA3; MAGEA4; MAGEAB; MAGEA9; MAGEBI7; MAGEBI; MAGEB2; MAGEBB; MAGECI; MAGEC2; MAGEC3; MAGEDI; MAGED2; MAGED4B; MAGED4; MAGEEI; MAGEHI;

MAGEL2; MAG; MAGII; MAGI2; MAGI3; MAGTI; MAKIB; MAK; MAL; MALL; MALRDI; MALTI; MAMLI; MAML2; MAML3; MAMLOI MANIAI;

MANIBI; MAN2AI MAN2A2; MAN2BI; MAN2CI MANBA; MANEA; MANF; MANSCI; MAOA; MAOB; MAPIO; MAPIA; MAPIS; MAPILC3A;

MAPILC3B2; MAPILC3B; MAPIS; MAP2; MAP2KI; MAP2K2; MAP2K3; MAP2K4; MAP2K5; MAP2 B; MAP2K7; MAP3KIO; MAP3KII; MAP3KI2; MAP3KI3; MAP3KI4; MAP3KI9; MAP3KI; MAP3K2; MAP3K3; MAP3K4; MAP3K5; MAP3 KB; MAP3K7CL; MAP3K7; MAP3K8; MAP3K9; MAP4; MAP4KI; MAP4K2; MAP4K3; MAP4K4; MAP4K5; MAPG; MAP7; MAPS: MAPKIO; MAPKII; MAPKI2; MAPKI3; MAPKI4;

MAPKI5; MAPKI; MAPK3; MAPKB; MAPK7; MAPKB; MAPK8IPI; MAPK8IP2; MAPK8IP3; MAPK9; MAPKAPI: MAPKAPK2; MAPKAPK3;

MAPKBPI: MAPREI; MAPRE2; MAPRE3; MAPT: MARCI; MARC2: MARCHI; MARCH2-, MARCH5; MARCHB; MARCH7; MARCH8; MARCKS;

MARCO; MARKI; MARK2; MARK3; MARK4; MARS2; MARS; MARVELDI; MARVELD2; MARVELD3; MASI; MASIL; MASPI; MAST2; MAST4;

MASTL; MATIA; MAT2A; MAT2B; MATK; MATN2; MATN3; MATR3; MAU2; MAVS; MAX; MAZ; MB2IDI; MB2ID2; MBDI; MBD2; MBD3; MBD3L2; MBD4; MBD5; MBDB; MB; MBIP; MBL2; MBNLI; MBNL2: MBDATI; MBOAT2; MBDAT4; MBP; MBTDI; MBTPSI; MBTPS2; MCIR; MC2R; C3R; MC4R; MC5R; MCAM; MCAT; MCCCI; MCCCZ; MCCDI: MCC; MCEE; MCP2; NCF2L2; CF2L; MCFD2; MCHRI; MCHR2;

MCIDAS; MCMIO; CM2: MCM3AP; MCM3; MCM4; MCM5; MCMG; CM7; CM8: MCM9; CMBP; MCDLNI; MCOLN3; MCPHI; MCRSI;

MCTPI MCTP2; MCTSI: MCG; MCURI; MDCI; DFIC; DFI; MDGAI; MDGA2; MDHI: MDH2; MD; MDMI: MDM2; MDM4; MEL: ME2; ME3;

MEAI: MECDM; MECP2; MEDI2; MEDI2L; MEDI3; MEDI3L; MEDI4; MEDI5; MEOIG; MEDI7; MEDI9; MEDI; MED22; MED23; MED24; MED25: MED28; MED29; MED3D; MED4; MEDG; MED9; MEF2A; MEF2B; MEF2BNB-MEF2B; MEF2C; MEF2D; MEFV; MEGFIO; MEGFII; MEGF8;

MEGF9; MEI1; MEISI; MEIS2; MELK; MEMDI; MENI; MEDXI; MEOX2; MEPIA; MEPE; MERTK; MESDCI; MESDC2; MESP2; MEST; METAPID;

METAPI; METAP2; MET; METRN; METTLI3; METTLIG; METTLI7; METTLI8; METTLI; METTL2IA; METTL2IB; METTL2IC; METTL24; METTLE;

METTL7A; METTL9; MEX3B; MEX3C; MEX3D; MFAP3; MFAP4; MFAP5; MFGE8; MFHASI: MFI2; MFNI; MFRP; MFSDII: MFSDI2; MFSDI

MFSOG; MFSD7; MFS08; MGA; MGAM; MGARP; MGATI; MGAT2; MGAT3; MGAT4A; MGAT4B; MGAT4C; MGAT5B; MGAT5; MGEA5; MGLL; MGMEI; MGMT; MGP; MGRNf: MGSTI; MGST2; MGST3; MIA2; MIA3; MIA; MIBI; MIB2; MICA; MICAL2; MICALCL; MICB; MICUI; MICU3; MIDI;

MID2; MIENt: MIER2; MIER3; MIF; MILRI; MINA; MINKI; MINPPI; MIDX: MIPEP; MIP; MIPDLI; MIRI-IHG; MISI8BPI; MITF; MIXLI; MKIG7;

MKKS; MKLI; MKL2; MKLN1; MKNKI; MKNK2; MKRNI; MKRN3; MKSI; MLANA; MLCI; MLEC; MLFI; MLF2; MLHI: MLH3; MLKL; MLLTIO; MLLTI;

MLLT3; MLLT4; MLLTG; MLN; MLNR; MLPH; MLST8; MLX; MLXIP; MLXIPL; MLYCD; MMAA; MMAB; MMADHC; MMD2; MMD; MME; MMELI;

MMPID; MMPII; MMPI2; MMPI3; MMPI4; MMPI5; MMPIG; MMPI7; MMPI9; MMPI; MMP20; MMP2I; MMP24; MMP25; MMP2B: MMP28; MMP2; MMP3; MMP7; MMP8; MMP9; MMRNI; MMRN2; MMSI9: MMS22L; MNI; MNATI; MNDA; MNSI: MNT; MNXI; MDAPI; MDBIA; MDBIB;

MDB2; MOB3B; MOB4; MOBP; MOCDS; MOCSI; MOCS2; MOGATI; MOGAT2; MOGAT3; MOG; MOGS; MDK; MONIA; MONIB; MDN2; MDRC3;

MDRF4LI; MORNI; MORN2; MDRN5; MOS; MOVIOLI: MPCI; MPC2; MPDUt; MPDZ; MPEGI: MPG; MPHDSPHIO; MPHDSPHB; MPHDSPH8;

MPHDSPH9; MPI; MPLKIP; MPD; MPPI; MPP2; MPP3: MPP5; MPP7; MPPEI; MPPED2; MPRIP; MPST; MPVI7; MPVI7L2; MPZ; MPZLI;

MPZL2; MPZL3; MRI; MRAP2; MRAP; MRAS; MRCI MRC2; MREIIA; MREG; MRFAPI: MRGBP; MRGPRF; MRGPRXI; MRGPRX3; MRGPRX4; MRU; MRMI; MR0H2B: MRO; MRPLIO; MRPL1I; MRPLI3; MRPLI5; MRPLI7; MRPLI9; MRPLI; MRPL23; MRPL28; MRPL33; MRPL3B; MRPL3;

MRPL4D; MRPL4I; MRPL44; MRPL4B; MRPL52; MRPL9; MRPSII; MRPSI2; MRPSIG; MRPSI8B; MRPS22; MRPS23; MRPS28; MRPS30;

MRPS3I; MRPS33; MRPSB; MRPS7; MRPS9; MRRF; MRS2; MRVII; MS4AI2; MS4AI; MS4A2; MS4A3; MS4A4A; MS4AGA; MS4A8;

MSANTD3-TMEFFI; MSC; MSGNI; MSH2; MSH3; MSH5; MSHB: MSII; MSI2; MSLN; MSMB; MSMOI; MSMP; MSN; MSRI; MSRA; MSRB2;

MSRB3; MSTI; MSTIR; MSTN; MSTDI; MSXI; MSX2; MTIA; MTIB; MTIE: MTIF; MTIG; MTIH; MTIM; MTIX; MT2A; MT3; MT4; MTAI MTA2; MTA3; MTAP; MTBP; MTCHI: MTCH2; MTCLI; MTCPI; MTDH; MTERFI; MTFI MTFMT; MTFPI; MTGI; MTHFDI; MTHFDIL; MTHFD2; MTHFR;

MTHFS; MTIF2; MTIF3; MTL5; MTMI: MTMRII; MTMRI2; MTMRI4; MTMRI; MTMR2; MTMR3; MTMR4; MTMRG; MTMR7; MTMR8; MTMR9;

MTNRIA; MTNRIB; MTDI; MTPAP; MTPN; MTRFI; MTR; MTRNR2L7; MTRR; MTSSI; MTTP; MTDRN; MTUSI; MTUS2; MTXI; MTX2; MUCI2;

MDCI3; MUCI5; MUCI7; MUCI9; MUCI; MUC2D; MUC2I; MUC22; MUC2; MUC3A; MUC4; MOC5AC; MDC5B; MUCB; MDC7; MUCLI MUMI;

MDRC; MUS8I; MUSK; MOT; MUTYH; MVBI2B; MVD; MVK; MVP; MXI; MX2; MXDI; MXD3; MXD4; MXII; MXRA5; MYADM; MYBBPIA; MYB; MYBLI; MYBL2; MYBPCI; MYBPC2; MYBPC3; MYBPH; MYCBP2; MYCBPAP; MYC; MYCN; MYCTI; MYD88; MYDGF; MYEF2; MYEOV; MYF5;

MYFG; MYHIO; MYHII; MYHI3; MYHI4; MYHI5; MYHI MYH2; MYH3; MYH4; MYHG; MYH7B; MYH7; MYH8; MYH9; MYLI2A; MYLI2B; MYLI;

MYL2; MYL3; MYL4; MYLG; MYL7; MYL9; MYLIP; MYLK2; MYLK3; MYLK; MYLPF; MYNN; MYDID; MYDI5A; MYOIG; MY0I8B; MYDIA; MYOIB;

MYDIC; MYDID; MYDIE; MYDIF; MYD3A; MYD3B; MY05A; MY05B; MYD5C; MYDG; MYD7A; MYD7B; MYD9A; MYD9B; MYDCD; MYOC;

MYODI; MYDF; MYDG; MYDMI; MYOM2; MYOT; MYOZI; MYDZ2; MYDZ3; MYPN; MYRF; MYRFL; MYRIP; MYTI; MYTIL; MYZAP; MZBI MZFI; N4BPI; N4BP2LI; N4BP2L2; NAAID; NAAI5; NAAIB; NAA20; NAA25; NAA30; NAA35; NAA4D; NAA50; NAAGD; NAAA; NAALAD2; NAALAOLI; NAALADL2; NASI NAB2; NABPf; NABP2; NACA; NACCi; NACC2; NAOSYNI; NAEI; NAFI; NAGA; NAGK; NAGLU; NAGPA; NAGS;

NAIP; NALCN; NA PT; NANOG; NANDSI; NANDS2; NANOS3; NANS; NAPILI: NAPIL3; NAPIL4; NAPIL5; NAPA; NAPEPLD; NAPG; NAPRT;

NAPSA; NARFL; NARR; NA S; NATID; NATI4; NATI; NAT2; NATB; NAT8B; NATS; NAT8L; NAVI NAV2; NAV3; NBAS; MBEA; NBEALi;

NBEAL2; NBN; NBPF3; NBRI; NCALD; NCAMI; NCAM2; NCAN; NCAPD2; NCAPD3; NCAPG2; NCAPG; NCBP2; NCEHI; NCFI; NCF2; NCF4; 5 NCKI: NCK2; NCKAPI; NCKAPIL; NCKAP5; NCKIPSD; NCL; NCOAI; NCDA2; NCOA3; NCOA4; NCDA5; NCDAB; NCDA7; NCDRI; NCDR2;

NCRI; NCR2; NCR3; NCR3LGI; NCSI; NCSTN; NDC8D; NDEI; NDELI; NDFIPI; NDFIP2; NDNF; NDN; NDP; NDRGI; NDRG2; NDRG3; NDRG4;

NDSTI; NDST2; NDST3; NDST4; NDUFAID; NDUFAII; NDUFAI2; NDUFAI3; NDUFAI; NDUFA2; NDUFA5; NDUFAB; NDUFAB; NDUFABI;

NDUFAFI; NDUFAF2; NDUFAF3; NDUFAF4; NDUFAF5; NDUFAFB; NDUFBID; NDUFBII: NDUFB2; NDUFB3: NDUFB4; NDUFB5; NDUFBB;

NDUFB8; NDUFBB; NDUFC2; NDUFSI; NDUFS2; NDUFS3; NDUFS4; NDUFSB; NDUFS7; NDUFSB; NDUFV1; NDUFV2; NDUFV3; NEB; NEBL; ID NECABI; NECAB3; NECAPI; NEDDI; NEDD4; NEDD4L; NEDD8; NEDD9; NEFH; NEFM; NEILI; NEIL2; NEIL3; NE IO; NEKII; NEKI: NEK2; NEK3;

NEK4; NEKB; NE 7; NE 8; NEK9; NELFA; NELFB; NELFCD; NELFE; NELLI; NELL2; NEMF; NEDI; NES; NETI; NETOI; NETD2; NEW; NEU3;

NEURLI; NEURL2; NEURODI; NELIROD2; NEURDD4; NEURDDB; NEURDGI; NEURDG2; NEURDG3; NFI; NF2; NFAMI; NFASC; NFAT5; NFATCI

NFATC2; NFATC3; NFATC4; NFE2; NFE2LI: NFE2L2; NFE2L3; NFIA; NFIB; NFIC; NFIL3; NFIX; NFKBI; NFKB2; NFKBIA; NFKBIB; NFKBIE;

NFKBILI; NFKBIZ; NFRKB; NFSI: NFUI; NFYA; NFYB; NFYC; NGB; NGDN; NGEF; NGF; NGFRAPI; NGFR; NGLYI; NHEJI; NHLHI; NHLRCI 15 NHLRC3; NHP2; NHP2LI; NHS; NHSLI; NICNI; NIDI; NID2; NIF3LI; NIMIK; NIN; N1NJI: NINJ2; NINL; NIPAI; NIPA2; NIPAL3; NIPAL4; NIPBL;

NIPSNAPI; NIPSNAP3B; NISCH; NITI; NIT2; NKAIN2; NKAIN3; NKAP; NKDI; NKD2; NKG7; NKIRASI; NKIRAS2; NKRF; NKTR; NKXI-I; NKXI-2;

NKX2-I; NKX2-2; NKX2-3; NKX2-5; NKX2-B; NKX2-8; NKX3-I; NKXB-I; NKXB-2; NLEI; NLGNI; NLGN2; NLGN3; NLGN4X; NLGN4Y; NLK;

NLN; NLRC3; NLRC4; NLRC5; NLRPIO; NLRPII; NLRPI2; NLRPI3; NLRPI4; NLRPI; NLRP2; NLRP3; NLRP4; NLRP5; NLRPB; NLRP7; NLRP8;

NLRP9; NMB; NMBR; NMEI; NMEI-NME2; NME2; NME3; NME4; NME5; NMEB; NME7; NME8; NME9; NMI; N NATI; N NAT2; NMNAT3; NMS; NMTI; NMT2; N U; NMURI; NMUR2; NNAT; NNMT; NNT; NOAf; NOBI; NDBDX; NDC3L; NODI: NDD2; NODAL; NOG; NOLII NDL3; NDL4;

NOLB; NDL8; NOLCI; NDMI ND OI; NOND; NOPID; NDPI4; NDPIB; NOP2; NDP5G; NDPB; NOSIAP; NOSI; NDS2; NOS3; NOSIP; NDSTRIN;

NOTCHI; NOTCH2; NOTCH3; NOTCH4; NOTUM; NDVAI NOVA2; NOV; NOX1; NOX3; NOX4; NOX5; NOXAI NOXDI; NPAPI; NPAS2; NPAS3;

NPAS4; NPAT; NPB; NPBWRI; NPCI; NPCILI: NPC2; NPDCI; NPEPLI; NPEPPS; NPFF; NPFFR2; NPHPI; NPHP3; NPHP4; NPHSI NPHS2;

NPL; NPLOC4; NPMI; NPM2; NPNT; NPPC; NPRI; NPR2; NPR3; NPRL2; NPRL3; NPS; NPSRI; NPTN; NPTXI; NPTX2; NPTXR; NPVF; NPW; 5 NPYIR; NPY2R; NPY5R; NPY; NODI; NDD2; NROBI; NRIDI; NRID2; NRIH2; NRIH3; NRIH4; NRII2; NRII3; NR2CI NR2C2; NRZEI; NR2E3;

NR2FI; NR2F2; NR2FB; NR3CI; NR3C2; NR4AI; NR4A2; NR4A3; NR5AI: NR5A2; NRGAI; NRAP; NRARP; NRBPI; NRBP2; NRCAM; NRDI

NRDE2; NREP; NRFI; NRGI; NRG2; NRG3; NRG4; NRGN; NRIPI; NRIP2; NRK; NRL; NRM; NRNI NRPI; NRP2; NRSNI; NRSN2; NRTN; NRXNI;

NRXN2; NRXN3; NSA2; NSDI NSDHL: NSF; NSFLIC; NSGI; NSMAF; NSMCE2; NSMF; NSRPI; NSUN2; NSUN3; NSUN5; NSUN7; NT5CIB; NT5CIB-RDHI4; NT5C2; NT5C3A; NT5C; NT5DCI; NT5DC3; NT5E; NT5M; NTANI; NTF3; NTF4; NTHLI; NTM; NTNI; NTN4; NTNGI NTNG2; 0 NTPCR; NTRKI; NTRK2; NTRK3; NTS; NTSRI; NTSR2; NUAKI; NUAK2; NUBI; NUBPI; NUBP2; NUBPL; NUCBI; NUCB2; NUCKSI; NUDCD;

NUDCD3; NUDC; NUDTIO; NUDTII; NUDTI5; NUDTI9; NUDTI: NUDT2I; NUDT2; NUDT3; NUDTB; NUDT7; NUF2; NUFIPI; NUFIP2; NUGGC;

NUMAI; NUMB; NUMBU NUPI53; NUPI55; NUP205; NUP2IO; NUP2I4; NUP35; NUP37; NUP43; NUP50; NUPB2; NUP85; NUP88; NUP93;

NUPB8; NUPL2; NUPRI; NUSI; NUSAPI; NUTF2; NUTMI; NUTM2A; NUTM2B; NVL; NWDI; NXFI; NXF2B; NXF3; NXF5; NXN; NXNL1; NXNL2;

NXPEI; NXPE2; NXPE4; NXPHI: NXPH2; NXTI: NXT2; NYAP2; NYX; OARDI; OASI; OAS2; OAS3; OASL; OAT; OAZI; OAZ2; OBFCI; OBP2A; 5 OBSCN; OBSLI; OC90; OCA2; OCIADI; OCIAD2; OCLM; OCLN; OCM2; OCM; OCRL; ODAM; ODCI; ODFI; ODF3B; ODF4; OFCCt OFDI; OGDH; OGDHL; OGFDDI; OGFR; OGGI; OGN; OGT; OIPS; OIT3; OLAI; OLFMI; OLFM2; OLFM4; OLFMLZB; OLI6I; OLIG2; OL1G3: OLRI; OMD; OMG;

OMP; ONECUTI; ONECUT2; OPAI; OPA3; OPCML; OPHNI; OPNILW; OPNIMW2; OPNISW; OPN3; OPN4; OPN5; OPRKI; OPRU; OPRMI; OPTC;

OPTN; ORIDA2; ORIOA4; ORIOCI; ORIOJI; ORIOJ3; ORIOJ5; ORIOKI; ORIOK2; ORIOR2; ORIOT2; ORIOXI; ORIOZI; ORIIAI; ORI2D2;

ORI2D3; ORI3C3; OR13C4; ORI3FI: ORI3GI; ORI3JI; ORI4JI; ORIAI; ORICI; ORID2; ORIEI; ORIE2; ORIJ2; ORIKI; ORIL8; ORIMI: ORINI; ORIN2; OR2A25; OR2AGI; OR2AK2; OR2AT4; OR2B2; OR2B3; OR2BB; OR2CI; OR2F2; OR2G2; OR2G3; OR2H2; OR2J2; OR2J3; OR2L2;

OR2M3; OR2M4; OR2M7; OR2S2; OR2TID; OR2TI2; OR2TI; OR2T2; OR2T33; OR2T4; OR2T5; OR2W1; ORZYI: OR2ZI; OR3AI; OR4AI5;

OR4CI2; OR4CI3; OR4CB; OR4DI0; OR4KI3; OR4P4; OR4S2; OR5IA2; OR5IA7; OR5IEI; OR5IE2; OR5IF2; OR5IVI; OR52B2: OR52B4;

OR52DI; OR52EG; OR52II; OR52I2; OR52KI; OR52K2; OR5ZMI: OR5DI8; OR5H2; OR5HB; OR5KI; OR5K2; OR5VI; ORGBI; OR6B2; ORBCI:

ORBFI; ORGK2; ORGK3; ORBK6; ORBNI; OR6N2; ORBXI ORGYI; OR7CI; OR7D2; OR7EZ4; OR8SI: ORBK2; OR9Q2; ORAM; ORAI3; ORAOVI; ORC3; ORC4; ORC5; ORCB; ORMI: ORM2; ORMDL3: OS3; OSBP2; OSBP; OSBPLID; OSBPLII; OSBPLIA; OSBPL2; OSBPL3;

OSBPL5; OSBPLB; OSBPL8; OSBPLB; OSCAR; OSERI: OSGEP; OSGINI; OSM; OSMR; OSRI; OSR2; OSTFI; OSTMI; OTC; OTDA; OTOF;

OTDG; OTDGL; OTOLI; OTOPI; OTOR; OTP; OTUBI; OTUDI; OTUD4; OTUD7A; OTUD7B; OTULIN; OTXI; OTX2; OVCA2; OVDLI; OVDL2; OXAIL;

OXCTI; OXERI; OXGRI; OXRI; OXSRI; OXT; OXTR; P2RXI; P2RX2; P2RX3; P2RX4; P2RX5: P2RXG; P2RX7; P2RYII; P2RYI2; P2RYI3;

P2RYI4; P2RYI; P2RY2; P2RY4; P2RYB; P2RY8; P3H2; P3H3; P3H4; P4HAI; P4HA2; P4HB; P4HTM; PA2G4; PABPCI; PA8PC3; 15 PABPC4L; PABPNI; PACRG; PACSI; PACS2; PACSINI; PACSIN2; PADI4; PAEP; PAFI; PAFAHIBI; PAFAHIB2; PAFAH2; PAGI PAGEI; PAGE4;

PAGE5; PAGRI; PAH; PAICS; PAIPI; PAIP2; PAKI; PAKIIPI; PAK2; PAK3; PAK4; PAKB; PAK7; PALB2; PALDI; PALLD; PALM2-AKAP2;

PAMIB; PAM; PAMRI; PAN2; PANS; PANKI; PANK2; PANXI; PANX2; PAOX; PAP07; PAPL; PAPOLA; PAPOLG; PAPPA2; PAPPA; PAPSSI;

PAPSS2; PAOR3; PAQR5; PADR7; PARD3B; PARD3; PAROBA; PARDGB; PARG; PARK2; PARL; PARMI; PARN; PARPI2; PARPI4; PARPI5;

PARPI; PARP2; PARP3; PARP4; PARPB; PARPB; PARPBP; PARS2; PARVA; PARVB; PARVG; PASDI; PASK; PATEI; PATZI; PAWR; PAXI:D PAX2: PAX3; PAX4; PAX5; PAXB; PAX8; PAXB; PAXIPI; PBK; PBLD; PBOVI PBRMI; PBXI; PBX2; PBX3; PBX4; PCBDI PCBD2; PCBPI;

PCBP2; PCBP3; PCBP4; PCCA; PCCB; PCDHID; PCDHIIX; PCDHIIY; PCOHI5; PCDHI7; PCDHI8; PCDHI9; PCDHI: PCDH2D; PCDH7;

PCDH8; PCDHB; PCDHAI; PCDHA4; PCDHAB; PCDHBI: PCDHB2; PCDHB3; PCDHB8; PCDHGAII; PCDHGA3; PCDHGB4; PCDHGBB;

PCDHGC3; PCEDIB; PCFII; PCGFI: PCGF2; PCGF3; PCGFB; PC; PCID2; PCKI; PCK2; PCLO; PCMI: PCMTI PCMTDI; PCNA; PCNT; PCNXL2;

PCNXL4; PCOLCE2; PCDLCE; PCP2; PCP4; PCSKI; PCSKIN; PCSK2; PCSK4; PCSK5; PCSKB; PCSK7; PCTP; PCYTIA; PCYTIB; PDAPI; 5 POCDIO; PDCDI; PDCDILG2; PDCD2; PDCD4; PDCD5; PDCD6; PDCOGIP; PDCD7; PDC; PDCL2; PDCL3; PDEIDA; PDEIIA; PDEI2; PDEIA;

PDEIB; PDEIC; PDE2A; PDE3A; PDE3B; PDE4A; PDE4D; PDE5A; PDEBA; PDEBB; PDEGC; PDEBD; PDEGG; PDEGH; PDE7A; PDE7B: PDE8A;

PDE8B; PDE9A; PDF; PDGFA; POGFB; POGFC; PDGFD; PDGFRA; PDGFRB; PDGFRL; PDHAI; PDHB; PDHX; PDIA2; PDIA3; PDIA4; PDIA5;

PDIAB; PDKI; PDK2; PDK3; PDK4; PDLIMI; PDLIM2; PDLIM3; PDLIM4; PDLIM5; PDLIM7; PDPI PDP2; PDPKI; PDPR; PDRGI; PDS5A;

PDS5B; PDSSI; PDSS2; PDXI; PDXDCI; PDXK; PDXP; PDYN; PDZD2; PDZD4; PDZD7; PDZD8: PDZKI; PDZRN3; PDZRN4; PEAI5; PEAKI; PEARI; PEBPI; PEBP4; PECAMI; PECR; PEGID; PEG3; PELII PELI2; PELPI; PEMT; PENK; PEPD; PERI; PER2; PER3; PERM!: PERP; PESI;

PETIOD; PETII7; PEXIIA; PEXI2; PEXI3; PEXI4; PEXIG; PEXI9; PEXI; PEX2B; PEX2; PEX3; PEX5; PEX5L; PEXB; PEX7; PF4; PF4VI; PFDN4;

PFDN5; PFDNB; PFKFBI; PFKFB2; PFKFB3; PFKFB4; PFKL; PFKM; PFKP; PFNI; PFN2; PGA3; PGA4; PGA5; PGA I; PGAM2; PGAM4; PGAPI;

PGAP2; PGAP3; PGBDI; PGBD5; PGC; PGD; PGF; PGGTIB; PGKI; PGK2; PGLS; PGLYRPI; PGLYRP2; PGLYRP3; PGLYRP4; PGM3; PGPEPI;

PGP; PGR; PGRMCI; PGRMC2; PHACTRI; PHACTR2; PHACTR3; PHB2; PHB; PHCI; PHC2; PHC3; PHEX; PHFIO; PHFII; PHFI2; PHFI9; PHFI; 5 PHF20; PHF2IA; PHF23; PHF2; PHF3; PHF5A; PHFB; PHF8; PHGDH; PHIP; PHKAI; PHKA2; PHKB; PHKGI; PHKG2; PHLDAI; PHLDA2; PHLDA3; PHLDBI PHLPPI; PHLPP2; PHOX2A; PHOX2B; PHPTI; PHRFI; PHTFf; PHTF2; PHYH; PHYHIP; PHYKPL; PII5; PUB; PI3; PI4 2A;

P14 2B; PI4KA; P14 B; PIASt; PIAS2; PIAS4; PIBFI; PICALM; PICKI; PIDI; PIDDI; PIEZDI; PIEZD2; PIFI; FIFO; PIGA: PIGF; PIGG; PIGL;

PIGM; PIGN; PIGD; PIGP; PIGB; PIGR; PIGT; PIGU; PIGW: PIGY; PIHIDI; PIK3API; PIK3C2A; PI 3C2B; PIK3C2G; PI 3C3; PIK3CA; PIK3CB; PIK3CD; PIK3CG; PIK3IPI; PIK3R1: PI 3R2; PIK3R3; PIK3R4; PI 3R5; PIKFYVE; PILRA; PIMI; PIM2; PIM3; PINI PIN4; PINXI PIP4K2A; PIP4K2B; PIP4K2C; PIP5KIA; PIP5 IB; PIP5 IC; PIP5KLI; PIP; PIPOX; PIR; PISD; PITPNA; PITPNMI; PITPNM3: PITRMI; PITXl; PITX2;

PITX3; PIWILI; PIWIL2; PIWIL3; PIWIL4; PJAI; PJA2; P DI; PKDILI; PKDIL2; PKDIL3; PKD2; PKD2LI; PKDCC; PKDREJ; PKHDI; PKIA; PKIB;

PKLR; PKM; PKMYTI; PKNI; PKN2; PKN3; PKNOXI; PKNOX2; PKPI; PKP2; PKP3; PKP4; PLA1A; PLA2GIO; PLA2GI2A; PLA2GI2B; PLA2GI5;

PLA2GIB; PLA2GIB; PLA2G3; PLA2G4A; PLA2G4B; PLA2G4C; PLA2G4D: PLA2G5; PLA2 GB; PLA2G7; PLA2RI; PLAA; PLACI; PLAC8;

PLAGI; PLAGLI; PLAGL2; PLAT; PLAU; PLAUR; PLBh PLBDI; PLCBI; PLCB2; PLCB3; PLCB4; PLCDI: PLCD3; PLCD4; PLCEI; PLCGI; PLCG2; PLCHI; PLCLI; PLCL2; PLCXD2; PLCXD3; PLCZI; PLDI; PLD2; PLD3; PLD4; PLD5; PLEC; PLEK2; PLEKHAI; PLEKHA2; PLEKHA5; PLEKHAB;

PLEKHA7; PLEKHBI; PLEKHDI; PLEKHFI; PLEKHF2; PLEKHGI; PLEKHG2; PLEKHG3; PLEKHG4; PLEKHGB; PLEKHH2; PLEKHMI; PLEKHDI;

PLEKH02; PLEK; PLG; PLGLB2; PLINI; PLIN2; PLIN3; PLIN4; PLIN5; PLKi: PLK2; PLK4; PLK5; PLLP; PLN; PLOD2; PLPI; PLP2; PLRGI;

PLSI; PLS3; PLSCRI; PLSCR3; PLSCR4; PLSCR5; PLTP; PLVAP; PLXDCI; PLXDC2; PLXNAI; PLXNA2; PLXNA3; PLXNA4; PLXNB1; PLXNB3;

PLXNCI; PLXNDI; PM2DDI; PMAIPI PMCH; PMEL; PMEPAI; PMFI-BGLAP; PMFI; PML; PMMI; PMM2; PMP22; PMPCA; PMPCB; PMSI; PMS2; PMVK; PNCK; PNKD; PNKP; PNLDCI; PNLIP; PNLIPRP2; PNMAI; PNMA2; PNMT; PNN; PNDI; PNOC; PNP; PNPLA1; PNPLA2; PNPLA3;

PNPLA4; PNPLA5; PNPLAG; PNPLA8; PNPO; PNPTI; PNRCf; POCIA; POCIB; POC5; PDDXL; POFIB; POFUTI; POFUT2; POGK; POGLUTI;

POLAI; POLA2; POLB; PDLOI; POLD2; POLD3; PDLD4; POLDIP3; POLE2; POLE3; POLE4; POLE; POLG2; POLG; POLH; POLL POLK; POLL;

POLM; POLN; POLO; POLRIA; POLRIB; POLRIC; POLRID; POLR2A; POLR2B; POLR2C; POLR2D; POLR2E; POLR2F; POLR2G; POLR2H;

POLR2J; POLR2K; POLR2M; POLR3A; POLR3B; POLR3E; POLR3K; POLRMT; POMI2I; POMC; POMGNTI; POMGNT2; POMK; POMP; POMTI; POMT2; POMZP3; PONI PON2; PONS; POP1; POP4; POPDC3; PORCN; POR; POSTN; POTI; POTED; POTEF; POTEG; POTEH; POTEM;

POUIFI; POO2AFI; POU2FI; POU2F2; POU2F3; POU3F2; POU3F3; POU3F4; POU4FI; POU4F2; P004F3; POU5FIB; POU5FI: POUBFI:

POOBF2; PPAI; PPA2; PPAP2A; PPAP2C; PPAPDCIB; PPAPDC2; PPAPDC3; PPARA; PPARD; PPARGCIA; PPARGCIB; PPARG; PPAT; PPBP;

PPCDC; PPEFI; PPEF2; PPFIAI: PPFIA2; PPFIA4; PPFIBPI PPFIBP2; PPIA; PPIB; PPIC; PPID; PPIF; PPIG; PPILI; PPIL2; PPIL3; PPIP5I;

PPL; PPMIA; PPMIB; PPMID; PPMIE; PPMIF; PPMIG; PPMIH; PPMIK; PPMIL; PPMIM; PPMEI; PPOX; PPPICA; PPPICC; PPPIRIO; PPPIRII; PPPIRI2A; PPPIRI2B; PPPIRI2C; PPPIRI3B; PPPIRI3L; PPPIRI4A; PPPIRI4B; PPPIRI4C; PPPIRI5A; PPPIRI5B; PPPIRI7; PPPIRI8;

PPPIRIA; PPPIRIB; PPPIR2; PPPIR3A; PPPIR3B; PPPIR3C; PPPIR42; PPPIR7; PPPIR9A; PPP2CA; PPP2CB; PPP2RIA: PPP2RIB;

PPP2R2A; PPP2R2B; PPP2R2C; PPP2R3A; PPP2R3B; PPP2R4; PPP2R5A; PPP2R5B; PPP2R5C; PPP2R5D; PPP2R5E; PPP3CA;

PPP3CB; PPP3CC; PPP3RI; PPP3R2; PPP4C; PPP4RI; PPP5C; PPPBC; PPPBR2; PPPBR3; PPRCI; PPT2; PPY; PQBPI: POLC3; PRACI;

PRAC2; PRADCI; PRAF2; PRAMI; PRAME; PRAPI; PRBI; PRB2; PRB3; PRB4; PRCI; PRCC; PRCD; FRCP; PRDMIO; PRDMII; PRDMI3; PRDMI4; PRDMI; PRDM4; PRDM5; PRDMB; PRDM7; PRDM8; PRDM9; PROX2; PRDX3; PRDX4; PRDX5; PRDXG; PREB; PRELP; PREP;

PREPL; PREXI; PREX2; PRFI; PRG2; PRG4; PRHI; PRH2; PRICKLEI; PRICKLE2; PRICKLE4; PRIM1; PRIMAL PRIMPOL; PR AAI; PRKABI;

PRKACA; PRKACB; PRKACG; PRKABI: PRKAG2; PRKAG3; PRKARIA; PRKARIB; PRKAR2A; PRKAR2B; PRKCA; PRKCB; PRKCDBP; PRKCD;

PRKCE; PRKCG; PRKCH; PRKCI; PRKCQ; PRKCSH; PRKCZ; PRKDI; PRKD3; PRKOC; PRKGI; PRKG2; PRKRA; PRKRIR; PRKX; PRLH; PRL;

PRLHR; PRLR; PRMI: PRM2; PRM3; PRMTI; PRMT2; PRMT3; PRMT5; PRMT8; PRND; PRNP; PROC; PROCR; PRODH; PROK2; PRBKRI: PROKR2; PROLI; PROMI; PROM2; PROPL PROSI; PROSERI; PRBXI: PROZ; PRPFI9; PRPF3I: PRPF3BB; PRPF3; PRPF40A; PRPF4B; PRPF4; PRPFFJ; PRPF8; PRPH2; PRPH; PRPSI; PRPSILI; PRPS2; PRPSAPI; PRPSAP2; PRRII; PRRI3; PRRI5; PRRIB; PRR34; PRR3;

PRR5; PRR9; PRRCI PRRC2A; PRRC2C; PRRG4; PRRTI; PRRT2; PRRXI; PRRX2; PRSSI2; PRSSI6; PRSSI: PRSS2I; PRSS22; PRSS23;

PRSS27; PRSS2; PRSS33; PRSS35; PRSS3; PRSS5D; PRSS53; PRSS55; PRSS57; PRSS58; PRSS8; PRTFDCI; PRTG; P TN3; PRUNE2;

PRUNE; PRX; PRY; PSAP; PSATI; PSCA; PSD3; PSD4; PSD; PSENI PSEN2; PSENEN; PSBI; PSB2; PSG5; PSGB; PSG8; PSG9; PSIPI; 5 PSKHI; PSMAI; PSMA2; PSMA3; PSMA4; PSMAB; PSMA7; PSMBID; PSMBI; PSMB4; PSMB5; PSMBB; PSMB7; PSMBB; PSMB9; PSMCI;

PSMC2; PSMC3; PSMC3IP; PSMC4; PSMC5; PSMCB; PSMDIO; PSMDI2; PSMDI3; PSMDI4; PSMDI: PSMD2: PSMD3; PSMD4; PSMDB;

PSMD7; PSMD8; PSMDB; PSMEI; PSME2; PSME3; PSME4; PSMFI; PSMBI; PSMG2; PSMG3; PSORSIGI; PSORSIC2; PSPCI; PSPH; PSPN;

PSTPIPI; PSTPIP2; PTBPI; PTBP2; PTCDI; PTCHI PTCHDI; PTCHD4; PTCRA; PTDSSI; PTEN; PTER; PTFIA; PTGDR2; PTGDR; PTGDS;

PTGERI; PTGER2; PTGER3; PTGER4; PTGES2; PTGES3; PTGES; PTGIR; PTGIS; PTG i; PTGSI; PTGS2; PTHIR; PTH2; PTH2R; PTH; PTHLH; ID PTK2B; PTK2; PTKB; PTK7; PTMA; PTMS; PTN; PTDVI; PTP4AI; PTP4A3; PTPMTI; PTPNII; PTPNI2; PTPNI3; PTPNI4; PTPNI8; PTPNI;

PTPN2I; PTPN22; PTPN23; PTPN2; PTPN3; PTPN4; PTPN5; PTPNB; PTPN7; PTPN9; PTPRA; PTPRB; PTPRCAP; PTPRD; PTPRE; PTPRG;

PTPRH; PTPRJ; PTPRK; PTPRM; PTPRN2; PTPRN; PTPRD; PTPRfl; PTPRR; PTPRS; PTPRT; PTPRZI; PTRF; PTRHI; PTRH2; PTRHDI; PTS;

PTTGI: PTTGIIP; PHG2; PTX3; PUFBD; PDM2; PURA; PURB; PUSID; PUS1; PVALB; PVR; PVRLI; PVRL2; PVRL3; PVRL4; PWP2; PWWP2B;

PXDN; PXDNL; PXK; PXMP2; PXN; PXTI; PYCARD; PYCRI; PYDCI; PYDC2; PYGB; PYGL; PYGM; PYGDI; PYGD2; PYHINI PYROXDI; PYY; PZP; OARS; DDPR; OKI; QPCT; QPCTL; OPRT; QRFP; ORFPR; ORSLI; OSDXI; QSOX2; QTRTI; R3HCCI; R3HCCIL; R3HDMI; R3HDML;

RABIIA; RABIIB; RABIIFIPI; RABIIFIP2; RABIIFIP3; RABIIFIP4; RABIIFIP5; RABI2; RABI4; RABI5; RABI8; RABIA; RABIB; RAB20; RAB2I;

RAB22A; RAB23; RAB24; RAB25; RAB27A; RAB27B; RAB28; RAB2B; RAB2A; RAB3I; RAB32; RAB33B; RAB34; RAB35; RAB3B; RAB37;

RAB38; RAB39A; RAB39B; RAB3A; RA83D; RAB3GAPI; RAB3GAP2; RAB3ILI; RAB3IP; RAB40AL; RAB4DB; RAB4DC; RAB4A; RAB4B;

RAB5A; RAB5B; RAB5C; RABGA; RABBB; RABBC; RAB7A; RAB8A; RAB8B; RAB9A; RABACI; RABEPI; RABEP2; RABEPK; RABGAPIL; RABGEFI; RABIF; RABLB; RACI; RAC2; RAC3; RACGAPI; RADI7; RADI8; RADI; RAD2I; RAD2ILI; RAD23A; RAD23B; RAD50; RAD5IAPI:

RAD5IB; RAD5IC; RAD5ID; RA05I: RAD52; RAD54B; RAD9A; RAD9B; RAEI; RAETIE; RAETIL; RAFI; RAGI; RAG2; RAII4; RAIL RAI2; RALA;

RALB; RALBPI; RALGAPAI: RALGAPA2; RALGAPB; RALGDS; RALGPSL RALY; RALYL; RAMP RAMP2; RAMP3; RANBPIO; RANBPI7;

RANBPI; RANBP2; RANBP3; RANBP3L; RANBPB; RANBP9; RANGAP RANGRF; RAN; RAPIB; RAPIGAP2; RAPIGDSI; RAP2A; RAP2B;

RAPGEFI; RAPGEF2; RAPGEF3; RAPGEF4; RAPGEF5; RAPH RAPSN; RARA; RARB; RARG; RARRESL RARRES2; RARRES3; RARS2; RARS; 5 RASAI; RASA2; RASALL RASAL2; RASDL RASD2; RASEF; RASGEFIA; RASGEFIC; RASBRFI; RASGRF2; RASGRPI; RASGRP2; RASGRP3;

RASGRP4; RASIPI; RASLIOA; RASLIDB; RASLIIA; RASLIIB; RASLI2; RASSF RASSF2; RASSF3; RASSF4; RASSF5; RASSFB; RASSF7;

RASSF8; RAX2; RAX; RBICCI; RBI; RBAK; RBBP5; RBBPB; RBBP7; RBBP8; RBBP9; RBCKL RBFBXI; RBFDX2; RBFDX3; RBLI; RBL2;

RBMID; RBMI2; RBMI4; RBMI4-RBM4; RBMI5; RBMI7; RBM2D; RBM25; RBM2B; RBM27; RBM28; RBM38; RBM39; RBM3; RBM45;

RBM4B; RBM47; RBM4; RBM5; RBMB; RBM7; RBMS RBMS2; RBMS3; RBMX2; RBMX; RBMXL2; RBMYIAL RBPI; RBP2; RBP3; RBP4; RBPJ; RBPMS2; RBSN; RBXI; RC3HI; RCANI; RCAN2; RCBTBI: RCBTB2; RCHYI; RCLL RCN RCN2; RCBRI; RCSDL RCVRN; RD3; RDHIO;

RDHII; RDHI2; RDHI4; RDHIB; RDH5; RDH8; RD I; RDX; REC8; RECK; RECOL5; RECOL; REEPI; REEP2; REEP3; REEP5; REEPB; REGIA;

REGIB; REG3A; RELA; RELB; REL; RELN; REM RENBP; REN; REPSL REPS2; RERG; RERGL; REST; RET; RETN; RETNLB; RETSAT: REVI;

REV3L; REXDI; REXD2; REX04; RFCI; RFC2; RFC3; RFC4; RFC5; RFFL; RFK; RFPL RFTI; RFTNL RFTN2; RFWD2; RFWD3; RFX RFX2;

RFX3; RFX4; RFX5; RFXG; RFX8; RFXANK; RFXAP; RGCC; RBLI RGL2; RGL4; RGMA; RGMB; RGN; RGPD2; RGR; RGSID; RGSII; RGSI2; 5 RGSI3; RGSI4; RGSIB; RGSI7; RGSI8; RGSI9; RGSI; RGS20; RGS2I; RGS22; RGS2; RGS3; RGS4; RGS5; RGSB; RGS7BP; RGS7; RGS8; RBS9BP; RBS9; RGSLI; RHAB; RHBD02; RHBDD3; RHBDFI RHBDF2; RHBDLI RHBDL2; RHCE; RHCG; RHEB; RHEBLI RHNDI: RHOB;

RHBBTBI; RHDBTB2; RH08TB3; RHOD; RHDF; RHOG; RHDH; RHD; RHDJ; RHD11: RHDTI; RHDU; RHOV; RHOXFI RHDXF2: HPNI RHPN2;

RIBC2; RICI; RIC3: RICTOR; RIFI; RILP: RILPLI RIMBP2; RIMBP3C: RIMSI: RIMS2; RIMS4: RINI; RIN2; RIN3; INB1; RINTI; RIDKI; RIOK2;

RI0K3; RIP I; RIP 2; RIP 3; RIP 4: RIPPLY2; RITI; RIT2: RITAI RLBPI RLF; RUM; RLNI RLN2; RLN3; RMDNI; R DN2; RMDN3; RMII; RMI2; RHNDI; RNASEII; RNASEI2; RNASEI3: RNASEI: RNASE2; RNASE3; RNASE4; RNASEB; RNASE7; RNASE8; RNASEB; RNASEHI;

RNASEH2A; RNASEH2B; RNASEH2C; RNASEK; RNASEL; RNASET2; RNDI; RND3; RNFI03-CHMP3; RNFI03; RNFIII; NF112: RNFII4;

RNFI23; RNFI25; RNFI28; RNFI30; RNFI35: RNFI38; RNFI39; RNFI44A; RNFI44B; RNFI4B; RNFI49; RNFI4; RNFI50; RNFI52; RNFI57;

RNFIG7; RNFIB8; RNFI7D; RNFI80; RNFI82: RNFI9A; RNF2D7; RNF20; RNF2I3; RNF2I4; RNF2IB; RNF24; RNF2; RNF3I: RNF34; RNF39;

RNF40; RNF4I; RNF43; RNF44; RNF4; RNF5; RNFB; RNF7; RNFS; RNGTT; RNHI; RNLS; RNMT; RNPEP; RNPEPLI; RNPSI; RDBOI; ROBD2; ROB03; ROB04; ROCKI: ROC 2; ROGDI; ROMI; RDPNIB; RDPNI; ROPNIL: RORI; ROR2; RORA; RORB; RDRC; RDSI; RPI RPILI; RP2; RP9;

RPAI; RPA2; RPA3; RPA4; RPAIN; RPAPI; RPE; RPGR; RPGRIPI; RPGRIPIL; RPH3A; RPH3AL: RPIA; RPLIOA; RPLIO; RPLIDL; RPLI2;

RPLI3; RPLI4; RPLI5; RPLI7; RPLI8; RPLI9; RPL2I; RPL23A: RPL23: RPL24; RPL27A; RPL29; RPL3D; RPL3I; RPL34;

RPL35; RPL3BA; RPL3BAL; RPL37A; RPL38; RPL39; RPL39L; RPL3; RPL4I; RPL4; RPLB; RPL7A; RPL7; RPLPD; RPLPI: RPNI; RPN2;

RPPI4; RPP2I; RPP25; RPP38; RPP4D; RPRDIA; RPRDIB; RPRD2; RPRM; RPSIO; RPSI4; RPSI5A; RPSIB; RPSI8; RPSI9BPI; RPS20; RPS24; RPS27A; RPS27; RPS27L; RPS29; RPS2; RPS3A; RPS3; RPS4X; RPS4YI RPSBKA2; RPSBKA3; RPSBKA4; RPSBKA5; RPSG AG;

RPSBKBI; RPSBKB2; RPS9; RPSA; RPTOR; RDCDI; RRAD; RRAGA: RRAS2: RRAS; RRBPl; RREBI; RRH; RRMI; RRM2B; RRN2; RRN3;

RRNADI; RRPIB; RRP1; RRP9; RRSI RSI RSAD2; RSFI: RSLIDI; RSL24DI; RSPHI: RSPH4A; RSPH9; RSPO2; RSPQ3: RSPO4; RSRCI;

RSRC2; RSRPI; RSUI; RTCB; RTELI; RTKN2; RTKN; RTLI: RTNI RTN2; RTN3; RTN4: RTN4IPI: RTN4R: RTP3; RTP4; RTTN; RUFYI; RUFY3; RUNDC3B; RUNXI; RUNXITI RUNX2; RUNX3: RUVBLI; RUVBL2; RXFPI: RXFP2; RXFP3; RXRA; RXRB; RXRG; RYBP; RYK: RYRI; RYR2; RYR3; SIDDAIO; SIODAII; SIODAI2: SIDOAI3; SIDOAI4; SIOOAIB; SIODAI; SIOOA2; SIDOA3; SIODA4: SIOOA5; SIODAB; SIOOA7A; SIOA7;

SIDDA8; SIODAB; SIOOB; SIDOP: SIDOZ; SIPR2; SIPR3; SIPR4; SIPR5; SAAI: SAA2: SAA4; SAALI; SAC3DI; SACMIL; SACS; SAEI; SAFB2;

SAFB; SAGEI; SAG; SALLI; SALL2; SALL3; SALL4; SAMDI4; SAMDI; SAMD4A; SAMD5; SAMD9; SAMD9L; SAMHDI; SAMM5D; SAMSNI;

SAP30BP; SAP3DL; SAPCDf; SAPCD2; SARIA; SARIB; SARDH; SARMI; SARNP; SARS2; SARTI; SART3; SASHI; SASH3; SATI; SAT2;

SATBI; SATB2; SATLI; SAVI SBDS; SBFI: SBF2; SBNOI; SBND2; SBSN; SC50; SCAFII; SCAFI; SCAF4; SCAF8; SCAI; SCAMP2; SCAMP5; SCAPER; SCAP; SCARA3; SCARAB; SCARBI; SCARB2; SCARF2; SCCPDH; SCD5; SCO; SCFDI; SCFD2; SCG2; SCG3; SCG5; SCGBIAI;

SCGBIDI; SCGBID2; SCGB2AI: SCGB2A2; SCGB2B2; SCGB3AI; SCGB3A2; SCGN; SCHIPI; SGIN; SCLTI; SCLY; SCML2; SCML4; SCNIBA;

SCNIIA; SGNIA; SCNIB; SCN2A; SCN2B; SCN3A; SCN3B; SCN4A; SCN4B; SCN5A; SCN7A; SCN8A; SCN9A; SCNMI; SCNN1A; SCNNIB;

SCNNID; SCNNIG; SCDI; SC02; SCPZDI SCPEPI SCRIB; SCRNI; SCT; SCTR; SCUBEI: SCUBE2; SCUBE3; SCYLI; SCYL3; SDCI; SDC2;

SDC3; SDC4; SDCBP2; SDCBP; SDCCAG3; SDCCAG8; SDF2; SDF2LI; SDF4; SDHAFI; SDHAF2; SDHAF4; SDHA; SDHC; SDHD; SDKI; SDK2; SDPR; SDR42EI; SDR9C7; SOS; SECIIA; SECIIC; SECI3; SECI4LI; SECI4L2; SECI4L3; SECIBB; SEC23A; SEC23B; SEC23IP;

SEC24A; SEC24B; SEC24C; SEC3IA; SECBIAI; SECBIB; SECB2; SECB3; SECISBP2; SECISBP2L; SECTMI; SELIL; SELE; SELENBPl; SELL;

SELP; SELPLG; SEMA3A; SEMA3B; SEMA3C; SEMA3D; SEMA3E; SEMA3F; SEMA3G; SEMA4A; SEMA4B; SEMA4D; SEMA4F; SEMA4G;

SEMA5A; SEMA5B; SEMABA; SEMABB; SEMABD; SEMA7A; SEMGI; SEMG2; SENPI; SENP2; SENP3; SENP5; SENPB; SENP8: SEPHSI;

SEPSECS; SERACI; SERFIA; SERGEF; SERINCI SERINC3; SERINC5; SERPI; SERP2; SERPINAIO; SERPINAII; SERPINAI2; SERPINAI: SERPINA3; SERPINA4; SERPINA5; SERPINAB; SERPINA7; SERPINA9; SERPINBIO; SERPINBI3; SERPINBI; SERPINB2; SERPINB3; SERPINB4; SERPINB5; SERPINBG; SERPINB7; SERPINB8; SERPINB9; SE PINCI; SERPINDI; SERPINEI; SERPINE2; SERPINE3; SERPINFI

SERPINF2; SERPINGI; SERPINHI; SERPINII; SERPINI2; SERTADI SERTA02; SESNI; SESN3; SETBPI; SETDIA; SETDIB; SETD2; SETD3;

SETD5; SETO7; SETD8; SETDB2; SET; SETMAR; SETX; SEZB; SEZBL2; SEZGL; SFI SF3AI; SF3BI; SF3B2; SF3BB; SFII; SFMBTI; SFMBT2;

SFRI; SFRPI; SFRP2; SFRP4; SFRP5; SFSWAP; SFT2D2; SFT203; SFTA2; SFTA3; SFTPAI; SFTPA2; SFTPB; SRPC; SFTPD; SFXNI; SFXN2; SFXN4; SGCA; SGCB; SGCD; SGCE; SGCG; SGCZ; SGIPI; SGKi; SGK223; SGK2; SGK3; SGMSI SGMS2; SGDLI; SGPLI; SGPPI; SGPP2;

SGSH; SGSM2; SGSM3; SGTA; SH2BI; SH2B2; SH2B3; SH2DIA; SH202A; SH2D3A; SH2D3C; SH2D4A; SH2D4B; SH3BGR; SH3BGRL2;

SH3BGRL; SH3BPI; SH3BP2; SH3BP4; SH3BP5; SH3DI9; SH3GLI; SH3GL2; SH3GL3; SH3 BPI; SH3PXD2A; SH3PXD2B; SH3RFI;

SH3RF3; SH3TC2; SH3YLI; SHANKI; SHANK2; SHANK3; SHARPIN; SHBG; SHB; SHCI; SHC2; SHC3; SHC4; SHCBPI; SHE; SHFMI; SHH;

SHISA2; SHISA3; SHISAB; SHISA9; SHMTI; SHMT2; SHOC2; SHDX2; SHDX; SHPK; SHPRH; SHQI; SHRDDM2; SHRDDM3; SHRODM4; SIAE; SIAHI; SIAH2; SIDTI; SIGIRR; SIGLECII; SIGLECI4; SIGLECI; SIGLEC5; SIGLEC7; SIGLECB; SIGLEC9; SIGMARI; SI; SIKI: SIK2; SI3;

Sill; SIMI; SIM2; SIN3A; SIN3B; SIPAI; SIPAIL2; SIPAIL3; SIRPA; SIRPBI; SIRPG; SIRTI; SIRT2; SIRT3; SIRT4; SIRT5; SIRTG; SIRT7; SITI;

SIVAI; SIX1; SIX2; SIX3; SIX4; SIX5; SIXG; SKAI; SKA2; SKAPI: SKAP2; SIL; SIV2L2; SIV2L; SKORI; SOR2; SKPI; SKP2; SLA2; SLA;

SLAIN2; SLAMFI; SLAMFB; SLAMF7; SLAMF8; SLBP; SLCIDAI; SLCIQA2; SLCIDAG; SLCIDA7; SLCIIAI; SLCIIA2; SLCI2AI; SLCI2A2;

SLCI2A3; SLCI2A4; SLCI2A5; SLCI2AB; SLCI2A7; SLCI2A8; SLCI2A9; SLCI3AI; SLCI3A2; SLCI3A3; SLCI3A5; SLCI4AI; SLCI4A2; SLCI5A1; SLCI5A2; SLCI5A4; SLCIBAIO; SLCIBAII; SLCIBAI2; SLCIBAI3; SLCIBAI; SLCIBA2; SLCIBA3; SLCIBAB; SLCIBA7; SLCIBA8;

SLCIBA9; SLCI7AI; SLCI7A2; SLCI7A3; SLCI7A4; SLCI7A5; SLCI7AB; SLCI7A7; SLCI7A8; SLCIBAI; SLCI8A2; SLCI8A3; SLCI9AI:

SLCI9A2; SLCI9A3; SLCIAI; SLCIA2; SLCIA3; SLCIA4; SLCIA5; SLCIAB; SLC2DAI: SLC20A2; SLC22AII; SLC2ZAI2; SLC22AI3; SLC22AI4;

SLC22AIB; SLC22AI7; SLC22AI8AS; SLC22AI8; SLC22AI; SLC22A23;

SLC22A24: SLC22A2; SLC22A3; SLC22A4; SLC22A5; SLC22AB;

SLC22A7; SLC22A8; SLCZ3AI; SLC23A2; SLC24AI; SLC24A2; SLC24A3; SLC24A4; SLC24A5; SLC25AIQ; SLC25AI2; SLC25AI3; SLC25AI4; SLC25AI5; SLC25AIB; SLC25AI8; SLC25AI9; SLC25AI; SLC25A20; SLCZ5A2I: SLC25A22; SLC25A23; SLC25A25;

SLC25A27; SLC25A2; SLC25A3B; SLC25A37; SLC25A38; SLC25A3; SLC25A40; SLC25A41; SLC25A42; SLC25A43; SLC25A45;

SLC25A4B; SLC25A47; SLC25A4; SLC25A52; SLC25A5; SLC25AB; SLC2BAI; SLC2BA2; SLC2BA3; SLC2BA4; SLC2BA5; SLC2BAB;

SLC2BA7; SLC2BA8; SLC2BA9; SLC27AI; SLC27A2; SLC27A3; SLC27A4; SLC27A5; SLC28AI; SLC28A2; SLC28A3; SLC29AI; SLC29A2;

SLC29A3; SLC29A4; SLC2AI0; SLC2AII; SLC2AI2; SLC2AI3; SLC2AI4; SLC2A2; SLC2A3; SLC2A4RG; SLC2A5; SLC2AB; SLC2A8; SLC2A9; SLC30AI0; SLC30AI: SLC30A3; SLC30A4; SLC30A5; SLC30AB; SLC30A7; SLC30A8; SLC30A9;

SLC3IAI: SLC3IA2; SLC32AI; SLC33AI; SLC34AI; SLC34A2; SLC34A3; SLC35AI; SLC35A2; SLG35A3; SLC35A4; SLC35B2; SLC35B4; SLC35CI: SLC35D3; SLC35FI;

SLC35F2; SLC35F3; SLC35F4; SLC35FB; SLC35GI; SLC35G2; SLC35G5; SLC35 GB; SLC3BAI; SLC3BA2; SLC37AI; SLC37A2;

SLC37A4; SLC38AI; SLC38A2; SLC38A4; SLC38A5; SLC38AB; SLC38A7; SLC38A8; SLC38A9; SLC39AID; SLC39AII; SLC39AI2;

SLC39AI3; SLC39AI4; SLC39AI; SLC39A2; SLC39A3; SLC39A4; SLC39AB; SLC39A7; SLC39A8; SLC39A9; SLG3AI; SLC3A2; SLC4DAI; SLC4IAI; SLC43AI; SLC43A2; SLC43A3; SLC44AI; SLC44A2; SLC44A4; SLC44A5; SLC45A2; SLC45A3; SLC45A4; SLC4BAI:

SLC4BA2; SLC47AI; SLC48AI; SLC4AIO; SLC4AII: SLC4AIAP; SLC4AI; SLC4A2; SLG4A3; SLC4A4; SLC4A5; SLC4A7; SLC4A9; SLC50AI;

SLC5IA; SLC5IB; SLC5ZAI; SLC52A2; SLC52A3; SLC5AII; SLC5AI2; SLC5AI: SLC5A2; SLC5A3; SLC5A4; SLC5A5; SLC5AB; SLC5A7;

SLC5A8; SLCBAII; SLCBAI2; SLCBAI3; SLGBAI4; SLCBAI5; SLCBAI8; SLCBAI9: SLCBAI: SLCBA20; SLCBA2; SLCBA4; SLCBA5; SLCBAB;

SLCBA7; SLCBAB; SLCBA9; SLC7AI0; SLC7AII; SLC7AI3; SLC7AI4; SLC7AI; SLC7A2; SLC7A3; SLC7A4; SLC7A5; SLC7AB; SLC7A7; SLC7A8; SLC7A9; SLCBAI: SLC8A2; SLC8A3; SLCBAI SLC9A2; SLC9A3; SLC9A3RI SLC9A3R2; SLC9A4; SLC9A5; SLCBAB; SLC9A7; SLCBAB; SLC9A9; SLC9BI: SLC9B2; SLC9CI; SLC9C2; SLGQIA2; SLCGIBi; SLC0IB3; SLCDIB7; SLCDICI: SLCD2AI: SLCD2BI; SLCB3AI;

SLCD4AI; SLCB4CI; SLCD5AI; SLCOBAI; SLFNI2; SLFNI2L; SLFNI4; SLFN5: SLITI: SLIT2; SLIT3; SLIT KI: SLITRK2; SLITRK3; SLITR 5;

SLITR B; SLK; SL AP; SLM02; SLN; SLPI; SLTM; SLU7; SLURPI: SLX4; SLX4IP; SMADI; SMAD2; SMAD3; S AD4; SMAD5; SMADB;

SMAD7; SMAD9; SMAGP; SMAPI; SMARCAI; SMARCA2; SMARCA4; SMARCA5; SMARCADI; SMARCALI; SMARCBI; SMARCCI; SMARCC2; SMARCDI; SMARCD3; SMARCEI; SMCIA; SMCIB; SMC2; SMC3; SMC4; SMC5; SMCB; SHCHDI; SMCD4; SMCP; SMEKI; SME 2; SMGI;

SMGB; SMG8; SMIMI5; SMIMI9; SMIM2D; SMIM2I; SMIM23; SMIM5; SMN2; SMDCI; SMBC2; SMO; SMQX; SMPDI; SMPD2; SMPD3;

SMPOL3A; SMPDL3B; SMPX; SMR3B; SMS; SMTN; SMTNLI; SMUI; SMUGI; SMURFI; SMURF2; SMYDI; SMYD2; SMYD3; SMYD4; SMYD5;

SNAII; SNAI2; SNAI3; SNAP23; SNAP25; SNAP29; SNAP47; SNAP9I; SNAPCI: SNAPC4; SNAPC5; SNCA; SNCAIP; SNCB; SNCG; SNDI;

SNEDI; SNF8; SNIPI: SNRK; SNRNP2BD; SNRNP27; SN NP7D; SNRPA; SNRPB; SNRPC; SN PDI: SNRPD3; SNRPE: SNRPF; SNRPN; SNTAI; SNTBI; SNTGI; SNTB2; SNUPN; SNURF; SNWI; SNXIO; SNXI2; SNXI4; SNXIB; SNXI8; SNXI9; SNXI: SNX2D; SNX24: SNX25;

SNX29; SNX2; SNX3D; SNX3; SNX5; SNX9; SB ATI; SDAT2; SDBP; SOCSI: SDCS2; SDCS3; SDCS4; SOCS5; SDCSB; SDCS7; SDDI;

SDD2; SDD3; SDHLHI; SDHLH2; SDN; SDRBSI; SDRBS2; SDRBS3; SDRCSI; SDRCS2; SDRCS3; SORD; SDRLI; SCSI: SOS2; SDSTDCI; SDST; SDXIO; SDXII: SDXI2; SDXI3; SDXI4; S0XI5; SDXI7; SDXI8; SOXI; S0X2I; SDX2; SDX3; SOX4; S0X5; SOXB; SDX7; SDX8; SDX9;

SPIOD; SPUD; SPI4D; SPI; SP2; SP3; SP4; SP5; SPB; SP7; SP8; SPAI7; SPACAI; SPACA3; SPAGIIA; SPAGIIB; SPAGIB; SPAGI; SPAG4; SPAG5; SPAGB; SPAG7; SPAG8; SPAG9; SPAM1; SPANXA2; SPANXBI: SPANXD; SPANXN4; SPARC; SPARCLI: SPAST; SPATAI3;

SPATAIB; SPATAI7; SPATAIB; SPATAIB; SPATA2D; SPATA2I; SPATA22; SPATA25; SPATA2; SPATA5; SPATA7; SPATA8; SPATA9; SPATCI;

SPC24; SPC25; SPCS3; SPDEF; SPDLI; SPDYA; SPECCI SPECCIL; SPEF2; SPEG; SPESPI; SPGII; SPG2D; SPG2I; SPG7; SPHKI; SPHK2;

SPHKAP; SPII; SPIB; SPIC; SPIDR; SPIN1; SPIN2A; SPINKI; SPINK2; SPIN4; SPIN5; SPIN6; SPINK7; SPINTI; SPINT2: SPNSI SPNS2;

SPOII: SPDCKI; SPDC 2; SPOC 3; SPDNt; SPDN2; SPDP; SPPI; SPP2; SPPL2A; SPPL2B; SPPL2C; SPPL3; SPREDI; SPRED2; SPRED3; SPR; SPRN; SPRRIA; SPRRIB; SPRR2A; SPRR2B; SPRR3; SPRTN; SPRYI; SPRY2; SPRY3; SPRY4; SPRYD7; SPSB3; SPSB4; SPTAI;

SPTANI; SPTB; SPTBNI; SPTBN2; SPTBN4; SPTBN5; SPTLCI; SPTLC2; SPTLC3; SPTSSB; SPTY2DI; SPZI: SOLE; SDRDL; SQSTMI: SRAI;

SRBDI; SRCAP; SRC; SRCINI; SRD5AI; SRD5A2; SRD5A3; SREBFI; SREBF2; SREKI; SREKIIPI; SRFBPI; SRF; SRGAP1; SRGAP2; SRGAP3;

SRGN; SRI; SRL; SRMS; SRPI4; SRPI9; SRPG8; SRP72; SRP9; SRPI; SRPK2; SRPRB; SRPR; SRPX2; SRPX; SRR; SRRMI; SRRM2;

SRRM4; SRRT; SRSFID; SRSFII; SRSFI2; SRSFI; SRSF2; SRSF3; SRSF4; SRSF5; SRSFB; SRSF7; SRSF9; SRXNI; SRY; SSI8; SSI8LI; SSB; SSBPI; SSBP2; SSFA2; SSHI; SSH2; SSMEMI; SSNAI; SSPN; SSPD; SSRI; SSR2; SSRPI; SSSCAI; SST; SSTRI; SSTR2; SSTR3;

SSTR4; SSTR5; SSUH2; SSXI SSX2B; SSX2IP; SSX4B; SSX5; STI3; STI4; STI8; ST20; ST3GALI; ST3GAL2; ST3GAL4; ST3GAL5;

ST3GALB; ST5; STBGALI; STBGAL2; STBGALNACI; STBGALNAC2; STGGALNAC4; STGGALNACB; ST7; ST7L; ST8SIAI; ST8SIA2; ST8SIA3;

ST8SIA4; ST8SIAB; STABI; STAB2; STAC3; STAC; STAGI; STAG2; STAGS; STAM2; STAMBP; STAMBPLI; STAM; STAPI; STAP2; STARDIO;

STARDI3; STARD3; STARD3NL; STARD5; STARD7; STARD8; STARD9; STAR; STATI; STAT2; STAT3; STAT4; STAT5A; STAT5B; STATE; STATH; STAU2; STBDI; STCI; STC2; STEAPI; STEAP2; STEAP3; STEAP4; STH; STIL; STIMI; STIM2; STIPI; ST IO; STKII: STKIIIP; ST I7A;

ST I7B; ST 19; STK24; ST 25; STK2B; STK3I; ST 32A; STK32B; ST 32C; STK33: ST 35; ST 38L; STK39; ST 3; STK4; STMN2; STMN3;

STMN4; STOM; STDMLI; STDML2; STOML3; STONI-GTF2AIL; STDNI: STOXI; STRAI3; STRAB; STRA8; STRADA; STRADB; STRAP; STRC;

STRN3; STRN4; STRN; STS; SH3A: STT3B; STUBI; STXII; STXIG; STXI7; STXI8; STXIA; STXIB; STX2; STX3; STX4: STX5; STXB; STX8;

STXBPI; STXBP2; STXBP4; STXBP5; STXBP5L; STXBPB; STYKI; STYX; SUBI; SUCLA2; SUCLGI; SUCLG2; SUCNRI: SUCO; SOOS3; SOFU; SUGCT; SOGPI; SUGTI; SULFI; SULF2; SULTIAI; SOLTIA2; SOLT1A4; SULTIBI; SULTIC2; SOLTIEI; SULT2A1: SOLT2BI; SULT4AI; SUMFI; SUMFZ; SUMDI; SUM02; SUM03; SUMD4; SUNI; SUN2; SUN3; SUOX; SUPT2DH; SUPT3H; SUPT4HI; SUPT7L; SUPV3LI; SURFI; SURF4;

SUSDI; SUSD2; SUSD4; SUSDB; SOV39HI; SUV39H2; SUV420H2; SUZI2; SV2B; SV2C; SVEPI SVIL; SVIP; SVOP; SWAP7D: SWTI; SYBU;

SYCEI; SYCEIL; SYCP2; SYCP2L; SYCP3; SYK; SYMPK; SYHI; SYN2; SYN3; SYNCRIP; SYNDIBI; SYNEI: SYNE2; SYNE4; SYNGAPI;

SYNGRI; SYNG 2; SYNJI: SYNJ2BP; SYNJ2; SYNM; SYNPO2; SYNPD; SYNPR; SYP; SYPLI; SYPL2; SYTII; SYT12; SYTI3; SYTI4; SYTI; SYT4; SYTB; SYT9; SYTLI; SYTL2; SmS; SYVNI; TAARI; TAAR2; TAAR5; TAARB; TABI; TAB2; TAB3; TACt; TAGS; TAC4; TACCI: TACC2;

TACC3; TACDI; TAC I; TACR2; TAGR3; TADAI; TADA2A; TADA3; TAFI5; TAFIB; TAFIC; TAFID; TAFI; TAF2; TAF3; TAF4B; TAF4; TAF5L;

TAFB; TAF7; TAF7L; TAF8; TAFB; TAGAP; TAGLN2; TAGLN; TALI; TAL2; TALDOI; TAMM4I; TANCI; TANC2; TANG02; TANK; TADKI; TAD 2;

TAD3; TAPI; TAP2; TAPBP; TAPBPL; TARBPI; TARBP2; TARP; TARS; TARSL2; TAS2RIB; TAS2RI3; TAS2RI4; TAS2RIB; TAS2RI;

TAS2R38; TAS2R5B; TAS2RBB; TAS2R9; TASPI; TATDNI; TAT; TAXIBPI; TAXIBP3; TAZ; TBATA; TBCIDI5; TBCIDIB; TBCIDI; TBCID2D; TBCID22A; TBCID22B; TBCID24; TBCID25; TBCID2; TBCID32; TBCID3C; TBCID3F; TBCID4; TBCID5; TBCID7; TBCID8; TBCID9; TBCA;

TBCC; TBCD; TBCE; TBCEL; M1: TBKBPI; TBLIX; TBLIXRI; TBLIY; TBL2; TBL3; TBP; TBPLI; TBPL2; TBRI; TBRGI; TBXID; TBXI8; TBXI9;

TBXI: TBX2D; TBX2I; TBX22; TBX2; TBX3; TBX4; TBX5; TBXB; TBXA2R; TBXASI; TCAIM; TCAP; TCEAI; TCEA2; TCEA3; TCEALI; TCEAL2;

TCEAL4; TCEAL7; TCEBI: TCEB2; TCEB3C; TGERGI; TCERGIL; TCFI2; TCFI5; TCFI9; TCF2B; TCF2I; TCF25; TCF3; TCF4; TCF7; TCF7LI;

TGF7L2; TCFL5; TCHH; TCHP; TGIRGI; TCLIA; TCLIB; TCNI; TCN2; TCDFI; TCPIB; TCPIDL2; TCPIILI; TCPI; TCTA; TCTEI; TCTNI; TCTN2; TCTN3; TDGFI; TDG; TDD2; TDPI; TDP2; TDRDI; TDRD3; TDRD5; TDRDB; TDRD7; TDRD9; TDR H; TDRP; TEADI; TEAD2; TEAD3; TEAD4;

TEC; TECPR2; TECR; TECRL; TECTA; TEF; TEFM; TEK; TEKTI; TEKT5; TEL02; TENMI; TENM2; TENM3; TENM4; TEPI; TEPP; TERFI; TERF2;

TERF2IP; TERT; TESC; TES; TESPAI TETI; TET2; TET3; TEXIDI; TEXII; TEXI4; TEXI5; TEX2B4; TEX29; TEX3D; TEX35; TEX40; TFAM; TFAP2A;

TFAP2B; TFAP2C; TFAP4; TFBIM; TFB2M; TFCP2; TFDPI: TFDP2; TFDP3; TFE3; TFEB; TFEC; TFFI: TFF2; TFF3; TFG; TF; TFIPII: TFPI2; TFPI;

TFPT; TFR2; TFRC; TGFA; TGFBI; TGFBIII; TGFB2; TGFB3; TGFBI; TGFBRI; TGFBR2; TGFBRAPI; TG; TGIFI; TGIF2-CZOorf24; TGIF2; TGIF2LX; TGMI; TGM2; TGM3; TGM4; TGM5; TGMB; TGM7; TGOLN2; TGSI: THADA; THAPIO; THAP1J: THAPI; THAP2; THAPG; THBD; THBSI;

THBS2; THBS3; THBS4; THEG; THEM4; THEM5; THE G; THEMIS2; THEMIS; THGIL; TH; THNSLI; THNSL2; THDCI; THDC2; THDC5; THDCB;

THDPI; THPD; THRA; THRB; THRSP; THSDI; THSD4; THSD7A; THYNI; TIAI: TIAFI TIALI; TIAMI: TIAM2; TICAMI; TICAM2; TICRR; TIFA;

TIGD2; TIGIT; TIMD4; TIMELESS; TIMMID; TIMMI7A; TIMM2I; TIMM22; TIMM23; TIMM44; TIMM5D; TIMM8A; TIMM8B; TIMMOCI; TIMPI;

TIMP2; TIMP3; TIMP4; TINAG; TINF2; TIPARP; TIPIN; TIPRL; TIRAP; TJPfc TJP2; TJP3; TKI; T 2; TKT; TKTLI; T TL2; TLDCI; TLEI; TLE2; TLE3; TLE4; TLEG; M1; TLK2; TLLI; TLL2; TLNI; TLN2; TLRIO; TLRI; TLR2; TLR3; TLR4; TLR5; TLRB; TLR7; TLR8; TLR9; TLXI; TLX2; TLX3;

TM4SFI; TM4SF2D; TM4SF4; TM4SF5; TMBSF2; TM7SF2; TM9SF2; TM9SF4; TMBIM4; TMBIMB; TMCI; TMC2; TMC3; TMC5; TMCB; TMC8;

TMCCI; TMCC2; TMCC3; TMCDI; TMCD4; TMCD5A; TMEDID; TMEDI; TMED2; TMED3; TMED4; TMED7; TMED7-TICAM2; TMEDB; TMEFFI;

TMEFF2; TMEMIOO; TMEMIDI; TMEMID5; TMEMIDBB; TMEMI08; TMEMII4; TMEMII5; TMEMII7; TMEMII; TMEMI2BA; TMEMI27; TMEMI28;

TMEMI32A; TMEMI32B; TMEMI32C; TMEMI32D; TMEMI32E; TMEMI34; TMEMI35; TMEMI38; TMEMI5DB; TMEMI5IA; TMEMI5IB; TMEMI54; TMEMI58; TMEMIGO; TMEMIBIB; TMEMIB3; TMEMIG5; TMEMIB9; TMEMI70A; TMEM17I; TMEMI73; TMEMI75; TMEMI7 GB; TMEMI78A;

TMEMI82; TMEMI83A; TMEMI84C; TMEMI85A; TMEMI87; TMEMI89; TMEMI89-UBE2VI; TMEMI8; TMEMI99; TMEM2DOA; TMEM205;

TMEM207; TMEM209; TMEM2I3; TMEM2I5; TMEM2IB; TMEM2I7; TMEM2I9; TMEM220; TMEM229A; TMEM23I; TMEM233; TMEM237;

TMEM24I; TMEM244; TMEM245; TMEM259; TMEM25; TMEM2BI; TMEM27; TMEM2; TMEM30A; TMEM30B; TMEM37; TMEM38A; TMEM38B;

TMEM39A; TMEM40; TMEM43; TMEM45A; TMEM47; TMEM50B; TMEM55A; TMEM57; TMEM5; TMEMBO; TMEMB2; TMEMB3A; TMEMB7; TMEM70; TMEM74B; TMEM79; TMEM87A; TMEM88; TMEM89; TMEM8B; TMEM95; TMEMB7; TMEM98: TMFI; TMIE; TMIGD2; TMIG03; TMLHE; TMODI: TMDD2; TMDD3; TMOD4; TMPO; TMPRSSIIA; TMPRSSIIB; TMPRSSIID; TMPRSSItE; TMPRSSI3; TMPRSSI5; TMPRSS2; TMPRSS3; TMPRSS4; T PRSSB: TMPRSS7; T PRSS9; TMSBID; TMSBI5B; TMSB4X; T TCI; TMTC2; TMTC3; TMXI: TMX2; TMX3; TNC;

TNFAIPI: TNFAIP2; TNFAIP3; TNFAIP6; TNFAIP8; TNFAIP8L2; TNFAIP8L3; TNF; TNFRSFIOA; TNFRSFfOB; TNFRSFIOC; TNFRSFIOD;

TNFRSFIIA; TNFRSFIIB; TNFRSF12A; TNFRSFI3B; TNFRSFI3C; TNFRSFI4; TNFRSFI8; TNFRSFIA; TNFRSFIB; TNF SF21; TNFRSFBB; TNFRSF8; TNFSFIO; TNFSFII; TNFSFI2; TNFSFI2-TNFSFI3; TNFSFI3B; TNFSFI3: TNFSFI4; TNFSFI5; TNFSFIB; TNFSF4; TNFSFB; TNFSF3;

TNIK; TNIPI; TNIP2; TNIP3; TNKI; TNK2; TNKS2; TNKS; TNMD; TNNCI; TNN; TNNII; TNNI2; TNNI3; TNNTI; TNNT2; TNNT3; TNPI; TNP2;

TNPBI; TNPB2; TNPD3; TNRCI8; TNRCBA; TNRCBB; TNR; TNSI; TNS2; TNS3; TNS4; TNXB; TOBI; TDB2; TOLLIP; TDMI TOMILl; TOMM20;

TOMM34; TOMM40; TOMM70A; TONSL; TOPI; TOP2A; TOP2B; TOP3A; TOP3B; TOPBPI; TOPORS; TORIA; TORIAIPI; TORIAIP2; TORIB;

TOR2A; TOX2; TOX3; TOX4; TOX; TP53AIPI; TP53BPI: TP53BP2; TP53; TP53III; TP53II3; TP53I3; TP53INPI; TP53INP2; TP53RK; TP53TG3C; TPB3; TP73; TPBG; TPCNI; TPCN2; TPD52; TPD52LI; TPD52L2; TPGS2; TPHI; TPH2; TPIt; TPKI; TPMI; TPM2; TPM3; TPM4;

TPMT; TPD; TPPI; TPP2; TPPP2; TPPP3; TPPP; TPRGI; TPR; TPRN; TPSABI; TPSB2; TPSDI; TPSGI; TPTI; TPTE2; TPTE; TPX2; TRA2A;

TRA2B; TRABD2A; TRABD; TRADD; TRAFI; TRAF2; TRAF3; TRAF3IPI; TRAF3IP2; TRAF4; TRAF5; TRAFB; TRAF7; TRAFDI: TRAKI; TRAK2;

TRAMI; TRAMILI; TRAM2; TRAPI; TRAPPCIO; TRAPPCII; TRAPPCI; TRAPPC2; TRAPPC4; TRAPPC9; TRATI; TRDMTI: TRDN; TREH; TREMI;

TREM2; TREMLI; TREML2; TRERFI; TREXI; TREX2; TRHDE; TRH; TRHR; TRIAPI; TRIBI; TRIB2; TRIB3; TRIMIO; TRIMII: TRIMI3; TRIMI5; TRIMIB; TRIMI7; TRIMZI TRIM22; TRIM23; TRIM24; TRIM25; TRIM2B; TRIM27; TRIM28; TRIM29; TRIM2; TRIM3I; TRIM32; TRIM34;

TRIM35; TRIM3B; TRIM37; TRIM38; TRIM39; TRIMS; TRIM40; TRIM42; TRIM44; TRIM50; TRIM5B; TRIM58; TRIM59; TRIMS; TRIMB2;

TRIMBB; TRIMB8; TRIMB9; TRIMB-TRIM34; TRIM7I; TRIM72; TRIM73; TRIM74; TRIMS; TRIMS; TRIDBP; TRIO: TRIPIO; TRIPII; TRIPI3;

TRIP4; TRIPB; TIQK; TRMTIOA; TRMTI2; TRMT1; TRMT44; TRMT5; TRMU; TRO; TROVE2; TRPAI; TRPCI; TRPC3; TRPC4AP; TRPC4; TRPC5;

TRPCG; TRPC7; TRPMI; TRPM2; TRPM3; TRPM4; TRPM5; TRPMB; TRPM7; TRPM8; TRPSI; TRPVfc TRPV2; TRPV3; TRPV4; TRPV5; TRPVB; TRRAP; TSACC; TSCI: TSC22DI; TSC22D3; TSC22D4; TSC2; TSEN2; TSEN34; TSEN54; TSFM; TSGIDI; TSGAIO; TSHR; TSHZI; TSHZ2;

TSHZ3; TSLP; TSNAX; TSN; TSPANIO; TSPANII; TSPANI2; TSPANI3; TSPANI4; TSPANIB; TSPANI8; TSPAN3I; TSPAH32; TSPAN33;

TSPAN4; TSPANB; TSPAN7; TSPAN8; TSPAN9; TSPEAR; TSPO2; TSPO; TSPYIO; TSPYI; TSPY3; TSPY4; TSPYLI; TSPYL2; TSPYL4;

TSPYL5; TSRI; TSSCI; TSSKIB; TSSK2; TSSK4; TSTA3; TSTDI; TST; TTB I; TTBK2; TTCI2; TTCI7; TTCI9; TTCI; TTC2IB; TTC28; TTC29;

TTC37; TTC39A; TTC39B; TTC3; TTC5; TTC6; TTC7A; TTC7B; TTC8; TTC9B; TTC9C: TTC9; TTFI; TTF2; TTII: TTI2; TTK; TTL; TTLLID; TTLLII; TTLLI2; mU; TTLL3; TTLL4; TTLL5; TTLLB; TTLL7; TTLL8; TTLL9; TTPA; TTR; TTYHI; TTYH2; TUBAIA; TUBAIB; TUBAIC; TUBA3D; TUBA4A;

TUBA8; TUBBI; TUBB2A; TUBB2B; TUBB3; TUBB4A; TUBB4B; TUBBB; TUBB; TUBDI; TUBEI: TUBGI; TUBG2; TOBGCP2; TUBGCP3;

TUBGCP4; TUBGCP5; TUBGCPB; TUB; TUFM; TUFTI; TULPI; TOLP2; TULP3; TOLP4; TUSCI; TUSC2; TUSC3; TUSC5; 1; TVP23B; TWFI;

TWISTI; TWIST2; TWSGI; TXK; TXLNG; TXN2; TXNDCI5; TXNDCIB; TXN0CI7; TXNDC5; TXN; TXNLI; TXNRD2; TXNRD3NB; TYK2; TYMP;

TYMS; TYR; TYRD3; TYROBP; TYRPI; TYSNDI TYWIB; U2AFI U2AF2; UACA; UAPI; UBAI; UBA2; UBA3; UBA7; UBACI; UBAC2; UBAPI; UBAP2; UBASH3A; UBASH3B; UBB; OBC; UBD; UBE2A; UBE2B; OBE2C; UBE2D1; UBE2D2; UBE2D3; UBE2EI; UBE2E2; UBE2E3; UBE2GI;

UBE2G2; UBE2H; UBE2I; UBE2JI; UBE2K; UBE2L3; UBE2LB; UBE2M; UBE2N; UBE202; UBE2DLI; UBE2R2; UBE2S; UBE2T; UBE2U;

UBE2VI; UBE2V2; UBE2Z; UBE3A; UBE3B; DBE3C; UBE4A; UBE4B: UBIAOI; UBL3; UBL4A; UBL5; UBL7; UBLCPI: UBNI; UBOX5; UBPI

UBOLNI; UBQLN2; UBOLNL; UBRI; UBR3; OBR4; OBR5; UBR7; UBTD2; UBTF; UBXNI; UBXN2A; UBXN2B; UBXN4; UCHLI; UCHL3; UCHL5;

UCKI; UCK2; UCKLI; UCMA; OCN2; UCN3; UCN; UCPI; UCP2; UCP3; UEVLD; UFOIL; UFLI; OFMI; UGCG; OGDH; UGGTI; UGGT2; UGP2; UGTIAIO; UGTIAI; UGTIA3; OGTIA4; UGTIA5; UGTIAB; UGTIA7; UGTIA8; UGTIA9; UGT2AI; UGT2A2; UGT2A3; DGT2BIO; UGT2BII; UGT2BI5; UGT2B28; UBT2B4; UGT2B7; UGT3A2; UGT8; UHMKI; UHRFIBPI; UHRFI; UHRF2; UIMCI; ULBPI; ULBP2; ULBP3; ULKI; ULK2; ULK3; ULK4;

UMBO; UMDOLI; UMPS; UNCII9; UNCI3A; UNCI3B; UNCI3C; UNCI3D; UNC45A; UNC45B; UNC5A; UNC5B; UNC5C; UNC5D; UNC79;

UNCB3A; UNC93BI; UNG; UPBI; UPFI; UPF2; UPF3A; UPF3B; UPKIA; UPKIB; UPK2; UPK3A; UPPI; UPP2; UPRT; UQCCI; UQCC2; UQCRB;

UDCRCI; UBCRC2; UQCRFSI UflCRO; URB2; URGCP; URII; URMI; URDCI; U OD; UROS; USBI; USEI USFi: USF2; USHIC; USHIG; USH2A; 5 USUI; USPIO; USPII; USPI2; USPI3; USPI4; USPI5; USPI7L2; USPI7L3D; USPI8; USP20; USP22; USP25; USP2B; USP28; USP2; USP32;

USP33; USP3B; USP37; USPS; USP4B; USP42; USP43; USP44; USP4B; USP48; USP49; USP4; USP5; USPB; USPBNL; USP7; USPS;

USP9X; USP9Y; USPLI; UST; UTFI; UTPI4A; UTPI4C; UTP2B; UTRN; UTS2B; UTS2; UTS2R; UTY; UVRAG; UVSSA; UXSI UXT; VACI4;

VAMPI; VAMP2; VAMP4; VAMP7; VAMP8; VANGL2; VAPA; VAPB; VARS2; VARS; VASHI; VASH2; VASP; VATI; VATIL; VAVI; VAV2; VAV3;

VAXI; VAX2; VBPI; VCAN; VCL; VCP; VCX2; VCX3A; VCX3B; VCX; VCY; VDACI; VDAC2; VDR; VEGFA; VEGFB; VEGFC; VENTX; VEPHI; VEZT; ID VGF; VGLLI; VGLL2; VGLL3; VGLL4; VHL; VHLL; VILI; VIM; VIPAS39; VIP; VIPRI; VIPR2; VIT; VKDRCI; VLDLR; VMA2I; VMDI; VMPI; VNIR2;

VNIR4; VNNI: VNN2; VNN3; VOPPI VPRBP; VPREBI; VPREB3; VPSII; VPSI3A; VPSI3B; VPSI3C; VPS2BA; VPS28; VPS33A; VPS33B;

VPS35; VPS3B; VPS37A; VPS37B; VPS37C; VPS39; VPS4I; VPS4A; VPS4B; VPS5I; VPS52; VPS53; VPS54; VPS72; VPS8; VRKI: VRK2;

VSIGID; VSIGI; VSIG2; VSIG4; VSNLI: VSTMI; VSXI; VSX2; VTAI; VTCNI; VTIIA; VTIIB; VTN; VWA2; VWA3A; VWA3B; VWA5A; VWA5BI:

VWA7; VWA8; VWCE: VWDE; VWF; WAPAL; WARS2; WARS; WASFI; WASF3; WASHI; WAS; WASL; WBPIL; WBP2; WBSCRI7; WBSCR22; 15 WDFY2; WDFY4; WDHDI; WDPCP; WDRII; WDRI2; WDRI7; WDRI9; WDRI; WDR2D; WDR2B; WDR3I; WDR34; WDR35; WDR3B; WDR37;

WDR43; WDR45B; WDR45; WDR4B; WDR48; WDR49; WDR4; WDR55; WDR5; WDRBD; WDRB2; WDRB4; WDRBB; WDR7D; WDR72;

WDR74; WDR7B; WDR78; WDR7; WDR8I; WDR83; WDR8B; WDR93; WEEI; WFDCI: WFDC2; WFSI; WHSCI; WHSCILI; WIFI: WIPFI WIPF2;

WIPF3; WIPII; WIPI2; WISPI; WISP2; WISP3; WLS; WNKI; WNK2; WNK3; WNK4; WNTIDA; WNTIDB; WNTII; WNTIB; WNTI; WNT2B; WNT2;

WNT3A; WNT3; WNT4; WNT5A: WNT5B; WNTB; WNT7A; WNT7B: WNT8A; WNT8B; WNT9A; WNT9B; WRAP53; WRB; WRN; WRN1P1; WSBI; WSCDI; WSCD2; WTI; WTAP; WTIP; WWCI; WWC2; WWDX; WWPI; WWP2; WWTRI; XAB2; XAFI; XAGEIB; XAGEIE; XBPI; XCLI; XCL2; XCRI;

XDH; XG; XIAP; XIRPI; XIRP2; XK; XKR4; XKRG; XKR9; XPA; XPC; XPNPEPI; XPNPEP2; XPNPEP3; XPDI; XPO4; XPD5; XPOB; XP07; XPRI;

XRCCI; XRCC2; XRCC3; XRCC4; XRCC5; XRCCBBPI; XRCCB; XRNI; XRN2; XRRAI; XXYLTI; XYLB; XYLTI; XYLT2; YAEIOI; YAPI; YARS2;

YBX2; YBX3; YDJC; YEATS4; YESI; YIFIA: YIPFI; YIPF3; YIPF5; YKTB; YLPMI; YMEILI; YPELI YPEL2; YPEL3; YPEL4; YPEL5; YTHDCI;

YTHDC2; YWHAB; YWHAE; YWHAG; YWHAH; YWHAD; YWHAZ; YYIAPI; YYI; ZACN; ZAK; ZAP7D; ZARI; ZARIL; ZASP; ZBEDI; ZBED4; ZBED5; 5 ZBPI; ZBTBID; ZBTBI2; ZBTBI4; ZBTBIB; ZBTBI7; ZBTBI8; ZBTB2D; ZBTB2I; ZBTB22; ZBTB24; ZBTB2; ZBTB32; ZBTB33; ZBTB34;

ZBTB38; ZBTB4I; ZBTB4B; Z8TB48; ZBTB49; ZBTB4; ZBTB5; ZBTB7C; ZBTB9; ZC2HCIB; ZC3HIO; ZC3HIIA; ZC3HI2C; ZC3HI2D; ZC3HI4;

ZC3HI5; ZC3H3; ZC3H4; ZC3H7A; ZC3H7B; ZC3HAVI; ZC3HCI; ZC4H2; ZCCHCII; ZCCHCI2; ZCCHCI4; ZCCHC2; ZCCHC3; ZCCHCB;

ZCCHC8; ZCRBI; ZCWPWI; ZDBF2; ZDHHCII; ZDHHCI2; ZDHHCI3; ZDHHCI4; ZDHHCI5; ZDHHCI7; ZDHHCI; ZDHHC2; ZDHHC7; ZDHHC8;

ZDHHC9; ZEBI; ZEB2; ZFAND3; ZFAND5; ZFANDB; ZFAT; ZFC3HI: ZFHX2; ZFHX3; ZFHX4; ZFPI; ZFP30; ZFP3B; ZFP3GLI ZFP3BL2; ZFP37; ZFP42; ZFP57; ZFPB4; ZFP82; ZFPBI; ZFPMI; ZFPM2; ZFR2; ZFR; ZFX; ZFY; ZFYVEI9; ZFYVE2I; ZFYVE2B; ZFYVE27; ZFYVE28; ZFYVE9;

ZGIBB; ZGLPI; ZGPAT; ZHXI; ZHX2; ZICI; ZIC2; ZIC3; ZIC4; ZIC5; ZIKI; ZIM2; ZKSCANI; ZKSCAN3; ZKSCAN7; ZMAT3; ZMAT4; ZMIZI;

ZMYM2; ZMYM3; ZMYM4; ZMYM5; ZMYNDID; ZMYNDII; ZMYND8; ZNFIDB; ZNFID7; ZNFID; ZNFII2; ZNFI2I: ZNFI3I; ZNFI32; ZNFI33; ZNFI4I;

ZNFI43; ZNFI4B; ZNFI48; ZNFI54; ZNFIBD; ZNFIB9; ZNFI75; ZNFI77; ZNFI82; ZNFI84; ZNFI85; ZNFI89; ZNFI97; ZNF2D2; ZNF2D5; ZNF2D;

ZNF2I2; ZNF2I4; ZNF2I5; ZNF2I7; ZNF224; ZNF22; ZNF23D; ZNF23B; ZNF239; ZNF23; ZNF248; ZNF24; ZNF253; ZMF2B0; ZHF2B3; 5 ZNF2G4; ZNF2BB; ZNF2B7; ZNF2B8; ZNF273; ZNF274; ZNF27B; ZNF277; ZNF280B; ZNF28DD; ZNF28I; ZNF282; ZNFZ8BB; ZNF29B: ZNF2; ZNF3DD; ZNF3II; ZNF3I8; ZNF32D; ZNF322; ZNF32; ZNF33D; ZNF33I; ZNF334; ZNF335: ZNF343; ZNF35D; ZNF354A; ZNF35;

ZNF3B5; ZNF3FJFJ; ZNF3B7; ZNF3B2; ZNF3B3: ZNF3S4; ZNF385A; ZNF385B; ZNF385D; ZNF39I: ZNF395; ZNF338; ZNF407; ZNF4ID;

ZNF415; ZNF4I3; ZNF4I; ZNF423; ZNF430; ZNF432; ZNF433; ZNF438; ZNF443; ZNF444; ZNF44; ZNF45I: ZNF45; ZNF4B2; ZNF4B9:

ZNF483; ZNF49D; ZNF4B2; ZNF4BB; ZNF5DI; ZNF5D7; ZNF5I2B; ZNF5I2; ZNF5I3; ZNF5IB; ZNF5I9; ZNF52I; ZNF53B; ZNF555; ZNF55B; 5 ZNF5B8; ZNF5B9; ZNF577; ZNF5SD; ZNF58I; ZNF582; ZNF583; ZNF585B; ZNF5B2: ZNF53B; ZNFBOB; ZNFBD7; ZNFB08; ZNFBI5;

ZNFBI8; ZNFB27; ZNFB23; ZNFB33; ZNFB44; ZNFB45; ZNFB4B; ZNFB52; ZNF654; ZNFSB4; ZNFBB7; ZNFBB8; ZNFG74; ZNFB7B; ZNFB78;

ZNFB83; ZNFB87; ZNFB83; ZNF703; ZNF7D4; ZNF7DB; ZNF7II; ZNF7I6; ZNF7I7; ZNF74B; ZNF74; ZNF750; ZNF7B3; ZNF7B4; ZNF7B5;

ZNF7B; ZNF774: ZNF77B; ZNF778; ZNF7B4; ZNF79; ZNF7; ZNF8DD: ZNF804A; ZNF804B; ZNF80; ZNF8I2; ZNF8I3; ZNFS1B; ZNF8I;

ZNF823; ZNF827; ZNF823; ZNFB3I: ZNF9I; ZNF32; ZNF33; ZNF38; ZNFX1; ZNHIT2; ZNHIT3; ZNRDI; ZN F3; ZPI; ZP4; ZPBP2; ZPLDI; ID ZPRI; ZRANB3; ZRSR2; ZSCANI8; ZSCAN22; ZSCAN2B; ZSCAN3I: ZSCAN32; ZSCAN9; ZSWIM2; ZSWIMB; ZWIO: ZWILCH; ZW1NT; ZYX;

ZZEFI; and ZZZ3.

In some embodiments, the therapeutic protein is selected from the group consisting of OTC, ASL, PAH, ABCB4, ABCB11, PAH, AGL, CFTR, MUT, PCCA, PCCB, ASS1, FAH, HMBS, ATP7B, PFIC2, LDLR, G6PC, AGXT, FXN, PAL, BCKDHA, BCKDHB, DBT, UGT1A1, SLC25A13, CD46, CFH, CFI, FIX, FVII, FVIII, C2, C3, C5, GCHD, CBS, MPI, LNL, SERPINGI, UROC1, SMPD1, GLA, GAA, GRHPR, ATP8B1, SERPINCI, PROS1, GBA, ACADVL, HFE, BCKDA, CDG1B, SERPINA1, BMPR2, ENG, ACVR1, SMAD4, BMPR9, HBB, FLCN, HSP1, AP3B1, HPS3, HPS4, HPS5, HPS6, DTNBP1, BLOC1S3, PLDN, AP3D1, BRAF, NF-1, SLC34A2, FBN1, COL1A1, COL1A2, COL1A3, COL5A1, COL5A2, ADAMTS2, PLOD1, TNXB, ABCA3, SP-B, SP-C, GBA, NPC1, NPC2, FOXF1, NKX2-1, SFTPB, SFTPC, ABCA3, CSF2RA, SFTPD, MUC5B, BMPR2, EIF2AK4, CSF2RB, DNAH5, DNAI1, DNAH11, AKR1D1, AMACR, ATP8B1, CYP7A1, FOXC2, GATA2, GHR, HSD3B7, IGFALS, IKBKG, JAGI, KIF11, NOTCHI, NOTCH2, NR1H4, SOX18, TJP2, P53, P73, P63, VIPAS39, VPS33B, EPO, ARGI, CPS1, NAGS, NOS, KRAS, OX40L, IL12, VEGF-A, MMA, TTR, PCSK9, AT, and ALAS1.

Lipid-Based Formulations

Lipid-based formulations have been increasingly recognized as one of the most promising delivery systems (also referred to herein as a delivery vehicle or carrier) for RNA due to their biocompatibility and their ease of large-scale production. Cationic lipids have been widely studied as synthetic materials for delivery of RNA. After mixing together, nucleic acids are condensed by cationic lipids to form lipid/nucleic acid complexes known as lipoplexes. These lipid complexes are able to protect genetic material from the action of nucleases and to deliver it into cells by interacting with the negatively charged cell membrane. Lipoplexes can be prepared by directly mixing positively charged lipids at physiological pH with negatively charged nucleic acids.

Conventional liposomes consist of a lipid bilayer that can be composed of cationic, anionic, or neutral (phospho) lipids and cholesterol, which encloses an aqueous core. Both the lipid bilayer and the aqueous space can incorporate hydrophobic or hydrophilic compounds, respectively. Liposome characteristics and behaviour in vivo can be modified by addition of a hydrophilic polymer coating, e.g. polyethylene glycol (PEG), to the liposome surface to confer steric stabilization. Furthermore, liposomes can be used for specific targeting by attaching ligands (e.g., antibodies, peptides, and carbohydrates) to its surface or to the terminal end of the attached PEG chains (Front Pharmacol. 2015 Dec. 1; 6:286).

Liposomes are colloidal lipid-based and surfactant-based delivery systems composed of a phospholipid bilayer surrounding an aqueous compartment. They may present as spherical vesicles and can range in size from 20 nm to a few microns. Cationic lipid-based liposomes are able to complex with negatively charged nucleic acids via electrostatic interactions, resulting in complexes that offer biocompatibility, low toxicity, and the possibility of the large-scale production required for in vivo clinical applications. Liposomes can fuse with the plasma membrane for uptake; once inside the cell, the liposomes are processed via the endocytic pathway and the genetic material is then released from the endosome/carrier into the cytoplasm. Liposomes have long been perceived as drug delivery vehicles because of their superior biocompatibility, given that liposomes are basically analogs of biological membranes, and can be prepared from both natural and synthetic phospholipids (Int J Nanomedicine. 2014; 9: 1833-1843).

Cationic liposomes have been traditionally the most commonly used non-viral delivery systems for oligonucleotides, including plasmid DNA, antisense oligos, and siRNA/small hairpin R A-shRNA). Cationic lipids, such as DOTAP, (1,2-dioleoyl-3-trimethylammonium-propane) and DOTMA (N-[1-(2,3-dioleoyloxy)propyl]-N,N,N-trimethyl-ammonium methyl sulfate) can form complexes or lipoplexes with negatively charged nucleic acids to form nanoparticles by electrostatic interaction, providing high in vitro transfection efficiency. Furthermore, neutral lipid-based nanoliposomes for RNA delivery as e.g. neutral 1,2-dioleoyl-sn-glycero-3-phosphatidylcholine (DOPC)-based nanoliposomes were developed. (Adv Drug Deliv Rev. 2014 February; 66: 110-116.)

Preferably, the expressible polynucleotides and heterologous mRNA constructs described herein are lipid formulated. The lipid formulation is preferably selected from, but not limited to, liposomes, lipoplexes, copolymers, such as PLGA, and lipid nanoparticles. In one preferred embodiment, a lipid nanoparticle (LNP) comprises:

(a) a nucleic acid,

(b) a cationic lipid,

(c) an aggregation reducing agent (such as polyethylene glycol (PEG) lipid or PEG-modified lipid),

(d) optionally a non-cationic lipid (such as a neutral lipid), and

(e) optionally, a sterol.

Preferably, the lipid nanoparticle formulation consists of (i) at least one cationic lipid; (ii) a neutral lipid; (iii) a sterol, e.g., cholesterol; and (iv) a PEG-lipid, in a molar ratio of about 20-60% cationic lipid: 5-25% neutral lipid: 25-55% sterol; 0.5-15% PEG-lipid.

Some examples of lipids and lipid compositions for delivery of an active molecule of this disclosure are given in WO/2015/074085 and U.S. Ser. No. 15/387,067, each of which is hereby incorporated by reference in its entirety. In certain embodiments, the lipid is a compound of the following Formula I:

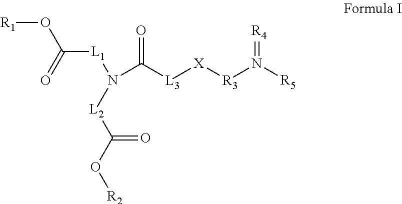

wherein
R₁ and R₂ both consist of a linear alkyl consisting of 1 to 14 carbons, or an alkenyl or alkynyl consisting of 2 to 14 carbons;
L₁ and L₂ both consist of a linear alkylene or alkenylene consisting of 5 to 18 carbons, or forming a heterocycle with N;

### X is S;

L₃ consists of a bond or a linear alkylene consisting of 1 to 6 carbons, or forming a heterocycle with N;
R₃ consists of a linear or branched alkylene consisting of 1 to 6 carbons; and
R₄ and R₅ are the same or different, each consisting of a hydrogen or a linear or branched alkyl consisting of 1 to 6 carbons;
or a pharmaceutically acceptable salt thereof.

The lipid formulation may contain one or more ionizable cationic lipids selected from among the following (also referred to herein as “ATX lipids”):

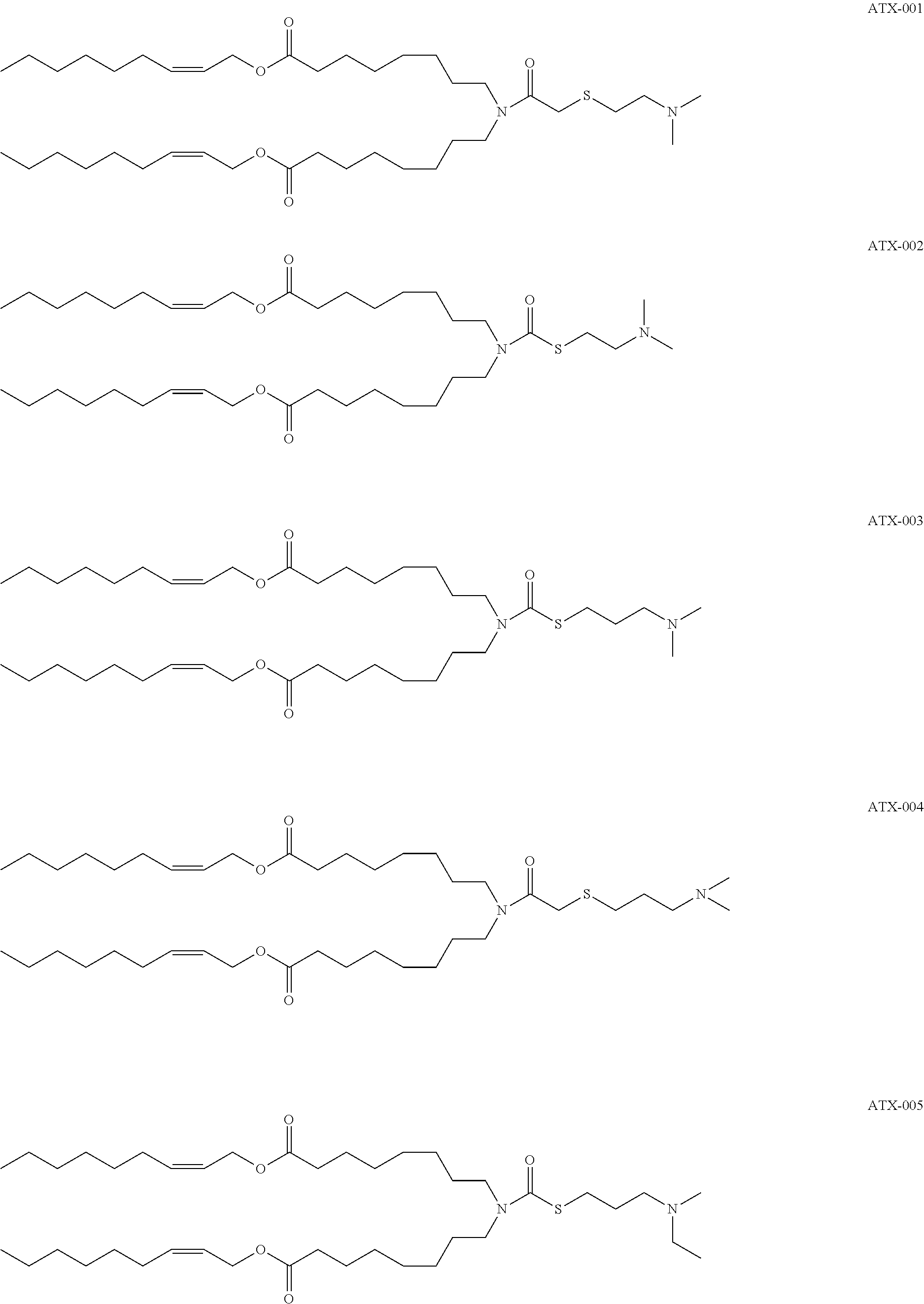

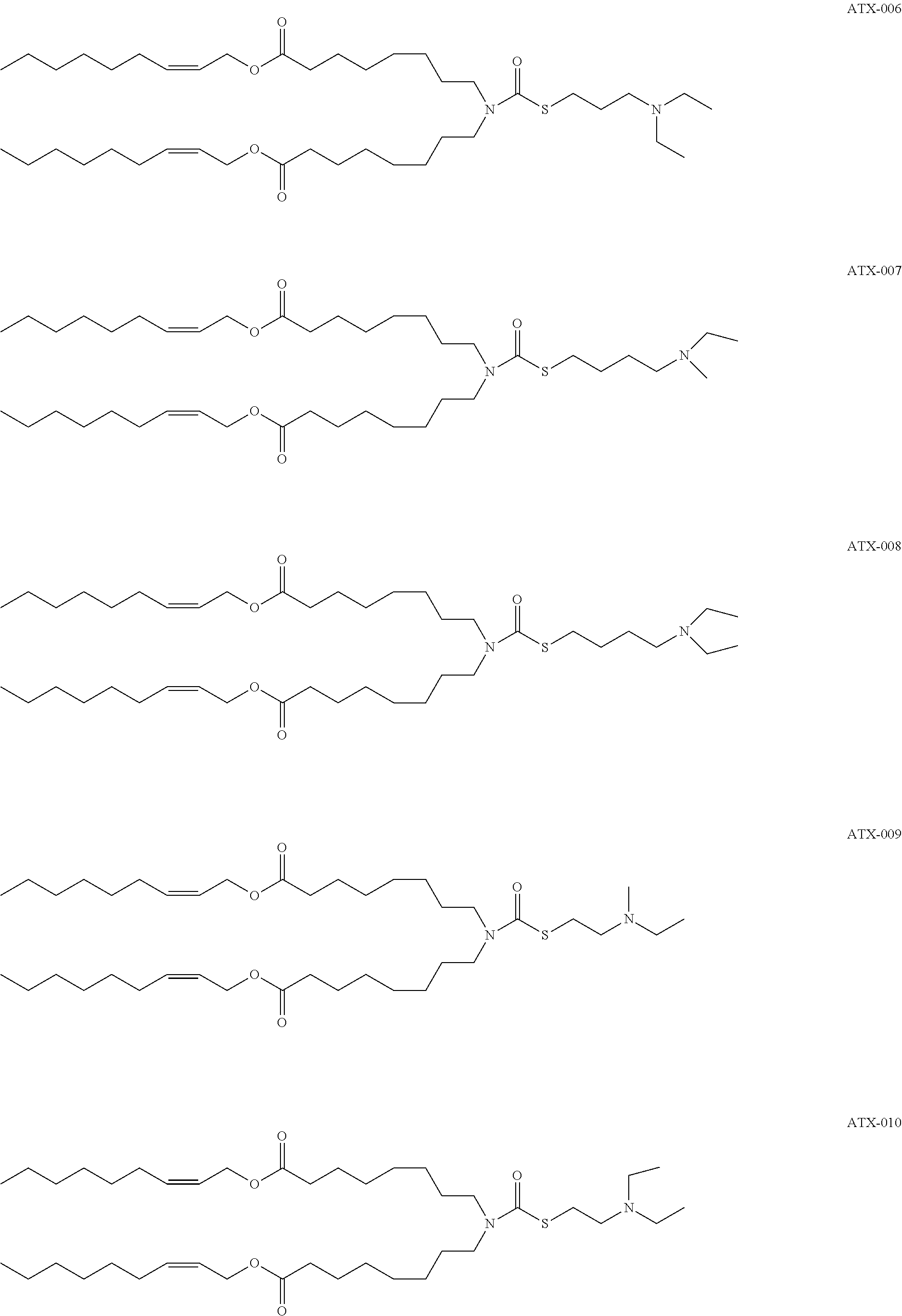

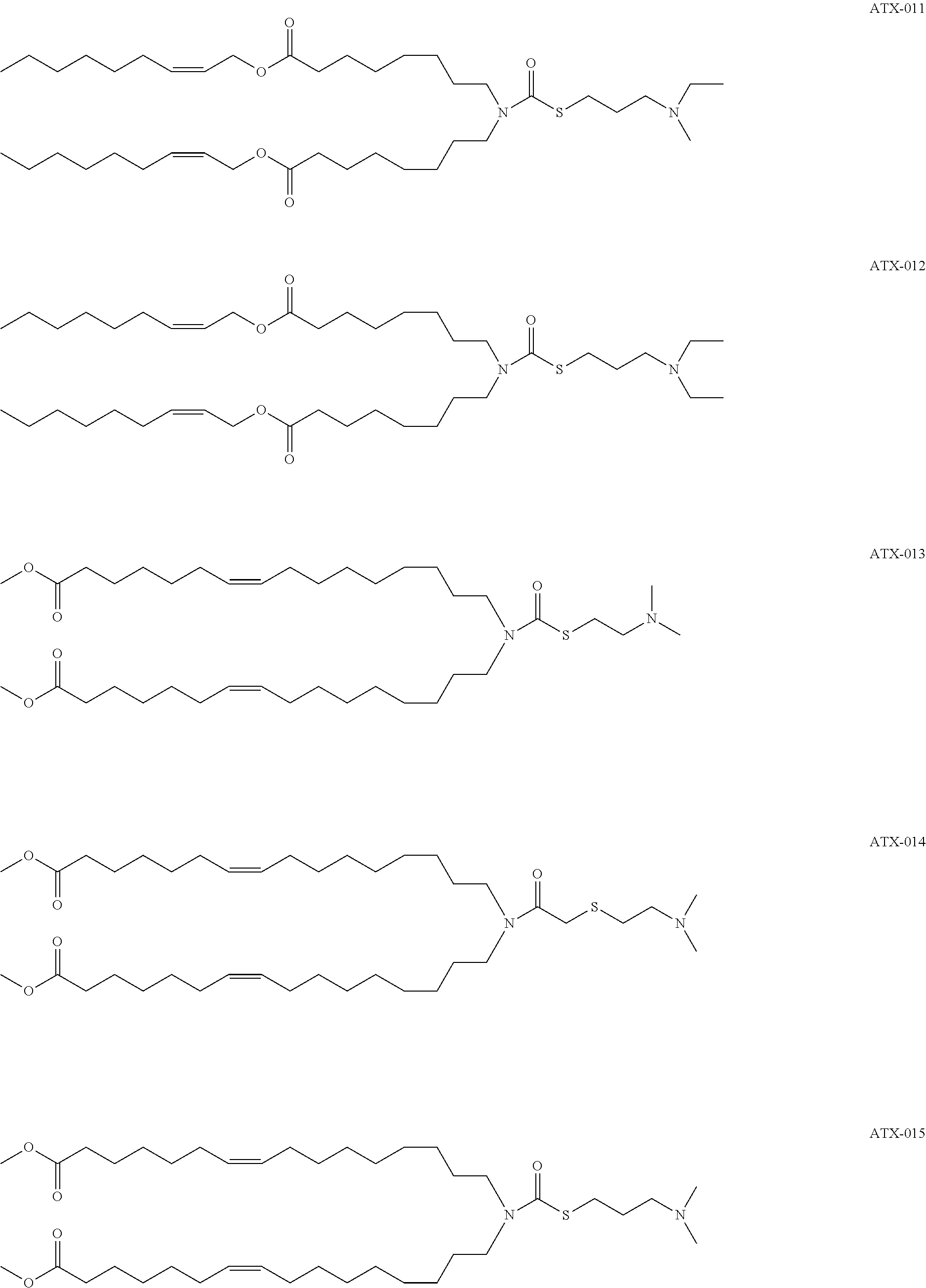

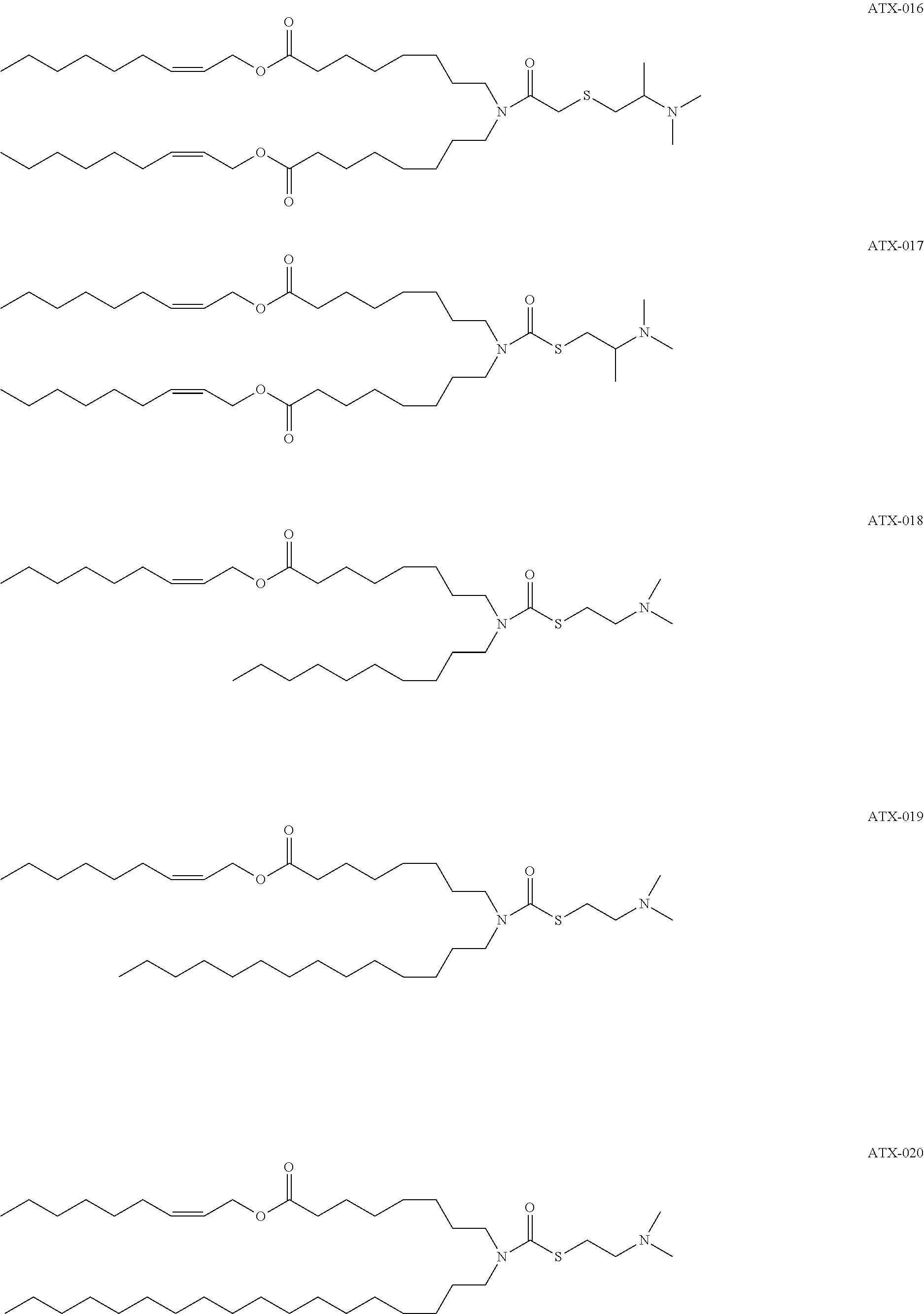

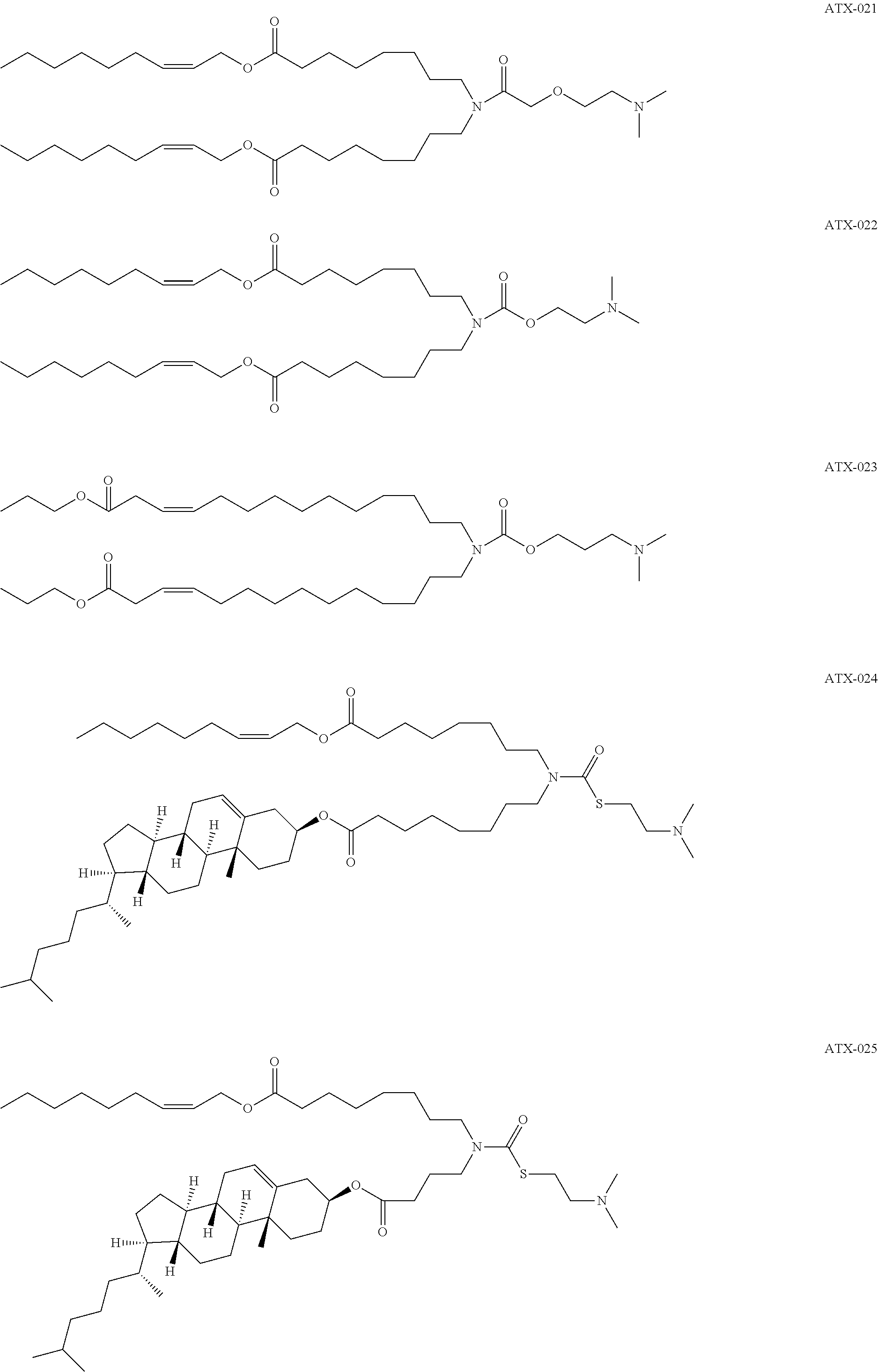

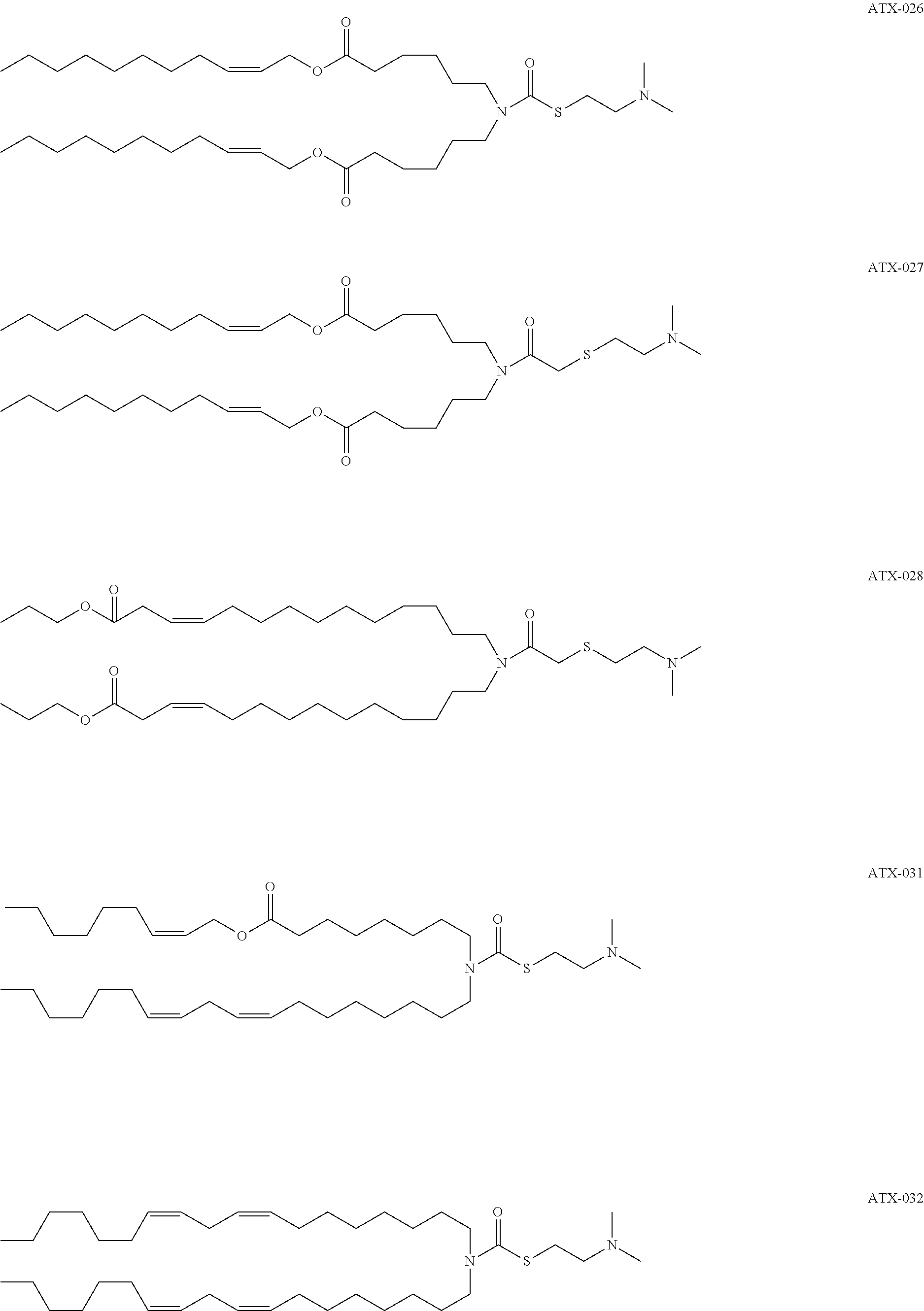

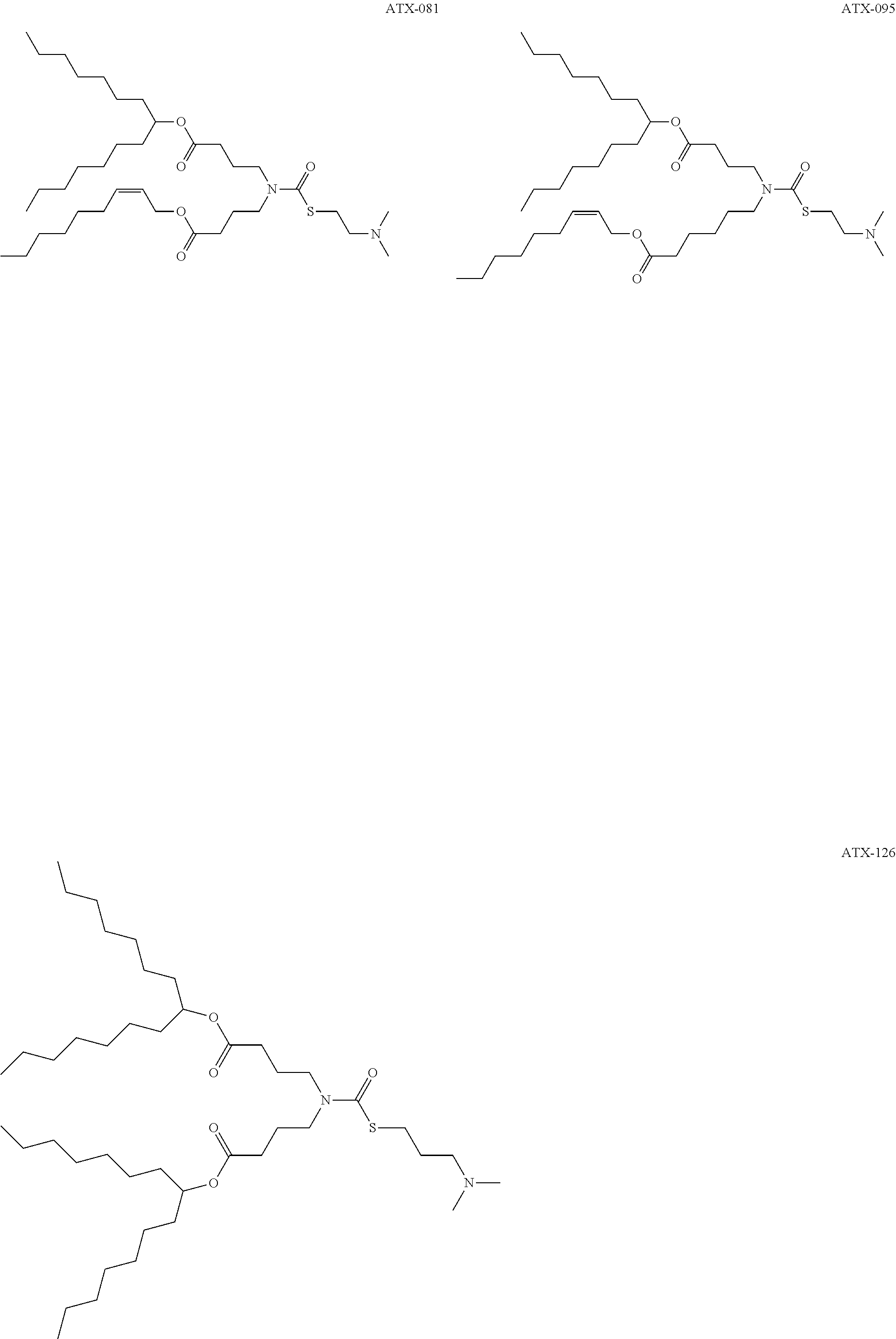 [SMILES: CCCCCC/C=C\COC(=O)CCCN(CCCC(=O)OC(CCCCCCC)CCCCCCC)C(=O)SCCN(C)C ; CCCCCC/C=C\COC(=O)CCCCCN(CCCC(=O)OC(CCCCCCC)CCCCCCC)C(=O)SCCN(C)C ; CCCCCCCC(CCCCCCC)OC(=O)CCCN(CCCC(=O)OC(CCCCCCC)CCCCCCC)C(=O)SCCCN(C)C]

The lipid nanoparticle preferably includes a cationic lipid suitable for forming a lipid nanoparticle. Preferably, the cationic lipid carries a net positive charge at about physiological pH. The cationic lipid may be, for example, N,N-dioleyl-N,N-dimethylammonium chloride (DODAC), N,N-distearyl-N,N-dimethylammonium bromide (DDAB), 1,2-dioleoyltrimethylammoniumpropane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (y-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.CI), 1,2-Dilinoleoyl-3-trimethylaminopropane chloride salt (DLin-TAP.CI), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DM A), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), (6Z,9Z,28Z,31Z)-heptatriaconta-6,9,28 31-tetraen-19-yl 4-(dimethylamino) butanoate (DLin-M-C3-DMA), 3-((6Z,9Z,28Z,31Z)-heptatriaconta-6,9,28,3 1-tetraen-19-yloxy)-N,N-dimethylpropan-1-amine (MC3 Ether), 4-((6Z,9Z,28Z,31 Z)-heptatriaconta-6,9,28,31-tetraen-19-yloxy)-N,N-dimethylbutan-1-amine (MC4 Ether), or any combination of any of the foregoing. Other cationic lipids include, but are not limited to, N,N-distearyl-N,N-dimethylammonium bromide (DDAB), 3P—(N—(N′,N′-dimethylaminoethane)-carbamoyl)cholesterol (DC-Choi), N-(1-(2,3-dioleyloxy)propyl)-N-2-(sperminecarboxamido)ethyl)-N,N-dimethylammonium trifluoracetate (DOSPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as, e.g., LIPOFECTIN (including DOTMA and DOPE, available from GIBCO/BRL), and Lipofectamine (comprising DOSPA and DOPE, available from GIBCO/BRL).

Other suitable cationic lipids are disclosed in International Publication Nos. WO 09/086558, WO 09/127060, WO 10/048536, WO 10/054406, WO 10/088537, WO 10/129709, and WO 2011/153493; U.S. Patent Publication Nos. 2011/0256175, 2012/0128760, and 2012/0027803; U.S. Pat. No. 8,158,601; and Love et al, PNAS, 107(5), 1864-69, 2010.

Other suitable amino lipids include those having alternative fatty acid groups and other dialkylamino groups, including those, in which the alkyl substituents are different (e.g., N-ethyl-N-methylamino-, and N-propyl-N-ethylamino-). In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C14 to C22 may be used. Other scaffolds can also be used to separate the amino group and the fatty acid or fatty alkyl portion of the amino lipid.

Preferably, the LNP comprises the cationic lipid with formula (III) according to the patent application PCT/EP2017/064066. In this context, the disclosure of PCT/EP2017/064066 is also incorporated herein by reference.

Preferably, amino or cationic lipids of the disclosure have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the disclosure. In certain embodiments, the protonatable lipids have a pKa of the protonatable group in the range of about 4 to about 11, e.g., a pKa of about 5 to about 7.

The cationic lipid can comprise from about 20 mol % to about 70 or 75 mol % or from about 45 to about 65 mol % or about 20, 25, 30, 35, 40, 45, 50, 55, 60, 65, or about 70 mol % of the total lipid present in the particle. In another embodiment, the lipid nanoparticles include from about 25% to about 75% on a molar basis of cationic lipid, e.g., from about 20 to about 70%, from about 35 to about 65%, from about 45 to about 65%, about 60%, about 57.5%, about 57.10%, about 50% or about 40% on a molar basis (based upon 100% total moles of lipid in the lipid nanoparticle). In one embodiment, the ratio of cationic lipid to nucleic acid is from about 3 to about 15, such as from about 5 to about 13 or from about 7 to about 11.

### Pharmaceutical Compositions

Preferably, the disclosure provides pharmaceutical compositions containing a codon optimized mRNA encoding a modified human therapeutic protein of interest preferably formulated in a lipid delivery system or lipid carrier and preferably comprising pharmaceutically acceptable excipients. Pharmaceutical compositions disclosed herein preferably facilitate expression of mRNA in vivo.

Suitable routes of administration include, for example, oral, rectal, vaginal, transmucosal, pulmonary including intratracheal or inhaled, or intestinal administration; parenteral delivery, including intradermal, transdermal (topical), intramuscular, subcutaneous, intramedullary injections, as well as intrathecal, direct intraventricular, intravenous, intraperitoneal, or intranasal.

Preferably, the intramuscular administration is to a muscle selected from the group consisting of skeletal muscle, smooth muscle and cardiac muscle. In some embodiments the administration results in delivery of the mRNA to a muscle cell. In some embodiments the administration results in delivery of the mRNA to a hepatocyte (i.e., liver cell). In a particular embodiment, the intramuscular administration results in delivery of the mRNA to a muscle cell.

Preferably, mRNAs and lipid formulations thereof may be administered in a local rather than systemic manner, for example, via injection of the pharmaceutical composition directly into a targeted tissue, preferably in a sustained release formulation. Local delivery can be affected in various ways, depending on the tissue to be targeted. For example, aerosols containing compositions of the present disclosure can be inhaled (for nasal, tracheal, or bronchial delivery); can be supplied in liquid, tablet or capsule form for administration to the stomach or intestines, can be supplied in suppository form for rectal or vaginal application; or can even be delivered to the eye by use of creams, drops, or even injection. Formulations containing provided compositions complexed with therapeutic molecules or ligands can even be surgically administered, for example in association with a polymer or other structure or substance that can allow the compositions to diffuse from the site of implantation to surrounding cells. Alternatively, they can be applied surgically without the use of polymers or supports.

Pharmaceutical compositions may be administered to any desired tissue. In some embodiments, the modified therapeutic protein-encoding mRNA delivered by provided liposomes or compositions is expressed in the tissue in which the liposomes and/or compositions were administered. In some embodiments, the mRNA delivered is expressed in a tissue different from the tissue in which the liposomes and/or compositions were administered. Exemplary tissues in which delivered mRNA may be delivered and/or expressed include, but are not limited to the liver, kidney, heart, spleen, serum, brain, skeletal muscle, lymph nodes, skin, and/or cerebrospinal fluid.

Preferably, a pharmaceutical composition can contain a polynucleotide described herein such as a primary DNA construct or mRNA described herein within a viral or bacterial vector.

Preferably, the primary DNA construct for an mRNA described herein or an mRNA described herein can be formulated using one or more excipients to: (1) increase stability; (2) increase cell transfection; (3) permit a sustained or delayed release (e.g., from a depot formulation of the polynucleotide, primary construct, or mRNA); (4) alter the biodistribution (e.g., target the polynucleotide, primary construct, or mRNA to specific tissues or cell types); (5) increase the translation of encoded protein in vivo; and/or (6) alter the release profile of encoded protein in vivo.

In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, liposomes, lipid nanoparticles, polymers, lipoplexes, core-shell nanoparticles, peptides, proteins, cells transfected with primary DNA construct, or mRNA (e.g., for transplantation into a subject), hyaluronidase, nanoparticle mimics and combinations thereof.

Accordingly, the formulations described herein can include one or more excipients, each in an amount that together increases the stability of the primary DNA construct, or mRNA, increases cell transfection by the primary construct, or mRNA, increases the expression of polynucleotide, primary construct, or mRNA encoded protein, and/or alters the release profile of polynucleotide, primary construct, or mRNA encoded proteins. Further, the primary construct and mRNA of the present disclosure may be formulated using self-assembled nucleic acid nanoparticles.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient may generally be equal to the dosage of the active ingredient which would be administered to a subject and/or a convenient fraction of such a dosage including, but not limited to, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.10% and 99% (w/w) of the active ingredient.

Pharmaceutical formulations may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired.

Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the embodiments of the present disclosure, except insofar as any conventional excipient medium may be incompatible with a substance or its derivatives, such as by producing any undesirable biological effect or otherwise interacting in a deleterious manner with any other component(s) of the pharmaceutical composition.

### Therapeutic Uses

The mRNA sequences, primary DNA constructs that transcribe the mRNA sequences described herein and pharmaceutical compositions thereof provide numerous in vivo and in vitro methods and are useful to treat deficiency of a protein. The treatment may comprise treating a human patient with deficiency of a protein. Similarly, compositions described herein, may be used in vitro or ex vivo to study OTC deficiency in cell or animal-based models. For example, cells deficient for the protein of interest expression can be used to analyze the ability to restore expression and/or activity of the protein of interest, as well as the time period over which expression and/or activity persists. Such cells and animal models are also suitable to identify other factors involved in the pathway, whether binding partners or factors in the same biochemical pathway. In other embodiments, compositions described herein can be used to study or track mitochondrial delivery.

Polynucleotides described herein, such as a DNA construct or template or an mRNA sequence described herein can be delivered to patients or cells experiencing deficiency of the protein of interest. For example, the mRNA sequence can comprise SEQ ID NO: 119 encoding a modified protein of SEQ ID NO: 4. In another example, the DNA sequence comprises SEQ ID NO: 221 encoding a modified protein of SEQ ID NO: 4.

Following administration, the modified protein of interest is expressed in the cells or subject. The modified protein of interest can have a signal peptide that directs it for secretion or to be a membrane bound protein. Preferably, compositions described herein are delivered to a targeted organelle. In some embodiments, the organelle can be selected from the group consisting of a mitochondrion, a nucleus, a lysosome, a peroxisome, an endoplasmic reticulum, a Golgi apparatus, and a plasma membrane. In some embodiments, compositions described herein are delivered to liver cells.

Examples of mRNA sequences for use with these methods include those listed in Table 5. Exemplary cDNA templates used to transcribe the mRNA sequences described herein are listed in Table 6. Exemplary mRNA sequences for administering to patients for treatment of a protein deficiency are SEQ ID NOS: 1799 and SEQ ID NOS: 1921.

In some embodiments, a polynucleotide described herein encoding a modified therapeutic protein described herein can be used in a gene therapy. Gene therapy is a technique that uses genes to treat or prevent disease, and can be used to treat a disorder by inserting a gene into a subject's cells. In some embodiments a polynucleotide encoding a modified therapeutic protein described herein replaces a mutated gene that causes disease. In other embodiments, a polynucleotide encoding a modified therapeutic protein described herein is used to inactivate, or “knock out,” a mutated gene that is functioning improperly. In yet other embodiments, a polynucleotide encoding a modified therapeutic protein described herein introduces a new gene into a subject to help fight a disease.

The gene therapy methods can use a carrier or vector that is genetically engineered to deliver the gene. In some embodiments, the vector is a viral-based vector. The vector can be injected or given intravenously (by IV) directly into a specific tissue in the body, where it is taken up by individual cells. Alternatively, a sample of the subject's cells can be removed and exposed to the vector ex vivo. The cells containing the vector are then returned to the subject.

### Dosing

An effective dose of a mRNA, a protein or pharmaceutical formulations thereof of the present disclosure can be an amount that is sufficient to treat ORF protein deficiency in a cell and/or in a patient. A therapeutically effective dose can be an amount of an agent or formulation that is sufficient to cause a therapeutic effect. A therapeutically effective dose can be administered in one or more separate administrations, and by different routes. Generally, a therapeutically effective amount is sufficient to achieve a meaningful benefit to the subject (e.g., treating, modulating, curing, preventing and/or ameliorating phenylketonuria). For example, a therapeutically effective amount may be an amount sufficient to achieve a desired therapeutic and/or prophylactic effect. Generally, the amount of a therapeutic agent administered to a subject in need thereof will depend upon the characteristics of the subject. Such characteristics include the condition, disease severity, general health, age, sex and body weight of the subject. One of ordinary skill in the art will be readily able to determine appropriate dosages depending on these and other related factors. In addition, both objective and subjective assays may optionally be employed to identify optimal dosage ranges.

Methods provided herein contemplate single as well as multiple administrations of a therapeutically effective amount of an mRNA sequence described herein. Pharmaceutical compositions comprising an mRNA sequence encoding an ORF protein described herein can be administered at regular intervals, depending on the nature, severity and extent of the subject's condition. Preferably, a therapeutically effective amount an mRNA sequence of the present disclosure may be administered periodically at regular intervals (e.g., once every year, once every six months, once every four months, once every three months, once every two months, once a month), biweekly, weekly, daily, twice a day, three times a day, four times a day, five times a day, six times a day, or continuously.

Preferably, the pharmaceutical compositions of the mRNA of the present disclosure are formulated such that they are suitable for extended-release of the translatable compound encoding a modified protein described herein contained therein. Such extended-release compositions may be conveniently administered to a subject at extended dosing intervals. For instance, in one embodiment, the pharmaceutical compositions of the present disclosure are administered to a subject twice a day, daily or every other day. In some embodiments, the pharmaceutical compositions of the present disclosure are administered to a subject twice a week, once a week, every 10 days, every two weeks, every 28 days, every month, every six weeks, every eight weeks, every other month, every three months, every four months, every six months, every nine months or once a year. Also contemplated herein are pharmaceutical compositions which are formulated for depot administration (e.g., subcutaneously, intramuscularly) to either deliver or release an mRNA sequence encoding a modified protein described herein over extended periods of time. Preferably, the extended-release means employed are combined with modifications made to the translatable compound encoding a modified protein described herein to enhance stability.

A therapeutically effective dose, upon administration, can result in serum or plasma levels of the modified therapeutic protein of interest of 1-1000 pg/ml, or 1-1000 ng/ml, or 1-1000 pg/ml, or more. In some embodiments, administering a therapeutically effective dose of a composition comprising an mRNA sequence described herein can result in increased modified protein levels in a targeted tissue (e.g., liver, eyes, lungs, skin, etc.) of a treated subject. Preferably, administering a composition comprising a mRNA described herein results in a 5%, 10%, 20%, 30%, 40%, 50%, 60%, 70%, 80%, 90%, or 95% increase in modified protein levels in the targeted tissue relative to a baseline modified protein level in the subject prior to treatment. Preferably, administering a therapeutically effective dose of a composition comprising an mRNA described herein will result an increase in levels of the modified protein in the targeted tissue relative to baseline levels of the protein of interest in the subject prior to treatment. In some embodiments, the increase in modified protein levels in the targeted tissue relative to baseline levels of the protein of interest will be at least 5%, 10%, 20%, 30%, 40%, 50%, 100%, 200%, or more.

Preferably, a therapeutically effective dose, when administered regularly, results in increased expression of the therapeutic protein of interest in the liver as compared to baseline levels prior to treatment. Preferably, administering a therapeutically effective dose of a composition comprising an mRNA sequence described herein results in the expression of a modified protein level at or above about 10 ng/mg, about 20 ng/mg, about 50 ng/mg, about 100 ng/mg, about 150 ng/mg, about 200 ng/mg, about 250 ng/mg, about 300 ng/mg, about 350 ng/mg, about 400 ng/mg, about 450 ng/mg, about 500 ng/mg, about 600 ng/mg, about 700 ng/mg, about 800 ng/mg, about 900 ng/mg, about 1000 ng/mg, about 1200 ng/mg or about 1500 ng/mg of the total protein in the liver of a treated subject.

A therapeutically effective dose of an mRNA described herein in vivo can be a dose of about 0.001 to about 500 mg/kg body weight. For instance, the therapeutically effective dose may be about 0.001-0.01 mg/kg body weight, or 0.01-0.1 mg/kg, or 0.1-1 mg/kg, or 1-10 mg/kg, or 10-100 mg/kg. Preferably, a Lipid-enabled and Unlocked Nucleomonomer Agent modified RNA (LUNAR)-mRNA (see WO/2015/074085 and U.S. Ser. No. 15/387,067), encoding a modified protein described herein, is provided at a dose ranging from about 0.1 to about 10 mg/kg body weight.

Combinations

The cDNA primary constructs, mRNA or encoded modified therapeutic proteins described herein may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. Preferably, the methods of treatment of the present disclosure encompass the delivery of pharmaceutical, prophylactic, diagnostic, or imaging compositions in combination with agents that may improve their bioavailability, reduce and/or modify their metabolism, inhibit their excretion, and/or modify their distribution within the body. As a non-limiting example, mRNA disclosed herein, encoding a modified protein of SEQ ID NO: 4 may be used in combination with a pharmaceutical agent for the treatment of OTC deficiency. The pharmaceutical agent includes, but is not limited to one or more of: sodium phenylbutyrate, glycerol phenylbutyrate, sodium phenylacetate, sodium benzoate, arginine, citrulline, Multiple vitamins, calcium supplements or combined with low protein/high caloric diet. In general, it is expected that agents utilized in combination with be utilized at levels that do not exceed the levels at which they are utilized individually. In some embodiments, the levels utilized in combination will be lower than those utilized individually. In one embodiment, the combinations, each or together may be administered according to the split dosing regimens as are known in the art.

### EXAMPLES

### Example 1: Material and Methods

In Vitro Transcription Protocol

The mRNAs were synthesized in vitro using T7RNA polymerase-mediated DNA-dependent RNA transcription where uridine triphosphate (UTP) was substituted and unsubstituted with modified UTPs such as 5 methoxy UTP (5MeOU), N1-methoxy methyl pseudo UTP (N1-MOM), 5-hydroxy methyl UTP, 5-carboxy UTP, and mixture of modifications using linearized template for each UTR combination. The mRNA was purified using column chromatography, the DNA and double stranded mRNA contamination of all mRNAs was removed using an enzymatic reaction, and the mRNA was concentrated, and buffer exchanged.

Preparation of Lipid Encapsulated mRNA

Lipid encapsulated mRNA particles were prepared by mixing lipids (ATX lipid: DSPC: Cholesterol: PEG-DMG) in ethanol with mRNA encoding the desired therapeutic polypeptide dissolved in Citrate buffer. The mixed material was instantaneously diluted with Phosphate Buffer. Ethanol was removed by dialysis against phosphate buffer using regenerated cellulose membrane (100 kD MWCO) or by tangential flow filtration (TFF) using modified polyethersulfone (mPES) hollow fiber membranes (100 kD MWCO). Once the ethanol was completely removed, the buffer was exchanged with HEPES (4-(2-hydroxyethyl)-1-piperazineethanesulfonic acid) buffer containing 50 mM NaCl and 9% sucrose, pH 7.3. The formulation was concentrated followed by 0.2 μm filtration using PES filters. The mRNA concentration in the formulation was then measured by Ribogreen fluorimetric assay following which the concentration was adjusted to a final desired concentration by diluting with HEPES buffer containing 50 mM NaCl, 9% sucrose, pH 7.3 containing glycerol. The final formulation was then filtered through a 0.2 μm filter and filled into glass vials, stoppered, capped and placed at −70±5° C. The frozen formulations were characterized for their mRNA content and percent encapsulation by Ribogreen assay, mRNA integrity by fragment analyzer, lipid content by high performance liquid chromatography (HPLC), particle size by dynamic light scattering on a Malvern Zetasizer Nano ZS, pH and Osmolality.

### In-Cell Western (ICW)

96-well collagen plates were used to seed the cells at the appropriate density in Dulbecco's Modified Eagle Media (DMEM)/Fetal Bovine Serum (FBS) culture media. At the optimal confluence, cells were transfected with the targeted mRNAs diluted in the transfection reagent mix (MessengerMax and Opti-MEM). Cells were placed in the CO2 incubator and allowed to grow. At the desire timepoint, media was removed, and cells were fixed in 4% fresh paraformaldehyde (PFA) for 20 min. After that, fixative was removed, and cells were permeabilize in tris buffered saline with TWEEN (TBST) for 5 min several times. When permeabilization washes were complete, cells were incubated with a blocking buffer (ODYSSEY@ Blocking Buffer (PBS) (Li-Cor, Lincoln, Nebr.)) for 45 min. Primary antibody was then added and incubated for 1 h at room temperature. Cells were then washed several times in TBST and incubated for 1 h with a secondary antibody diluted in blocking buffer and containing a CellTag 700 stain. To finalize, cells were washed several times in TBST followed by a last wash in tris-buffered saline (TBS). The plate was imaged using the Licor detection system and data was normalized to the total number of cells labeled by the CellTag 700.

### Example 2: UTRs Screening in Hepa1,6 and Hep3B—Correlation at 24 h and 48 h

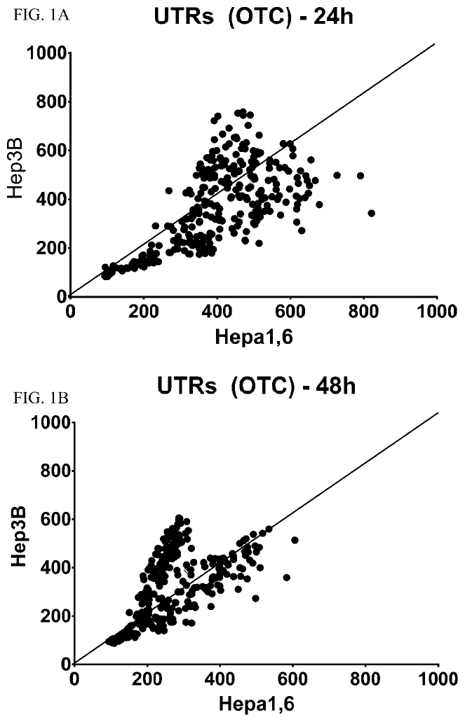

A UTR library was screened in vitro using mRNA construct #571 comprising the sequence of SEQ ID NO: 34, which relates to OTC, as CDS (coding sequence). In-Cell Western assays as described in Example 1 were used to transfect the different mRNAs into Hepa1,6 and Hep3B using commercially available transfection reagents. OTC protein expression levels were measured by near-infrared fluorescent imaging systems. Commercially available antibodies for OTC were used for detection. Untransfected and reference sequences were used as internal controls. FIG. 1, Panel A is a scatter plot of OTC protein expression levels found in Hepa1,6 and Hep3B cells at 24 hours. FIG. 1, Panel B is a scatter plot of OTC protein expression levels found in Hepa1,6 and Hep3B cells at 48 hours. The aim of the screening was to determine an UTR-specific impact on OTC expression levels in a human (Hep3B) and a mouse (Hepa1,6) liver cell line that will be beneficial to determine which UTRs would work best in both models, in particular, its translatability from mouse-to-human. Top expressing UTRs were used in further profiling studies.

### Example 3: Round 1 of Protein Stability Compounds Screened in Hepa1,6 and Hep3B at 24 h—Correlation

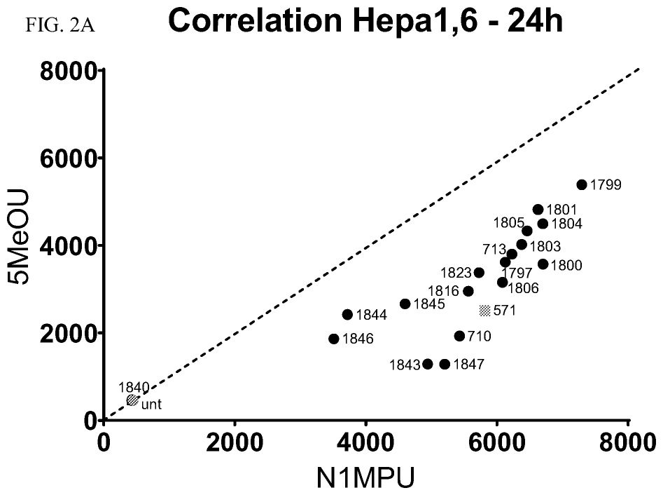

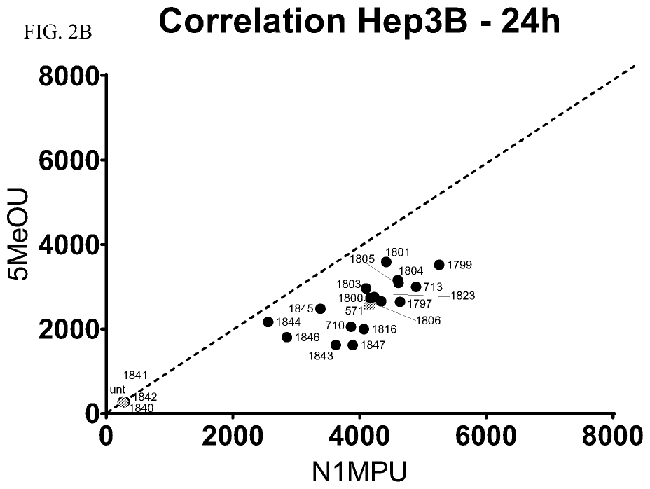

In vitro screening of certain mRNA constructs of Table 5 that were designed based on a protein-stability approach was performed. The mRNA constructs were tested in two different chemistries N1-methylpseudouridine (N1MPU) and 5-methoxyuridine (5MeOU) meaning that 100% of the uridines in each mRNA were N1MPU only or 5MeOU only (not a combination of 5MeOU or N1MPU). In-Cell Western (ICW) assays as described in Example 1 were used to transfect the different mRNAs into Hepa1,6 and Hep3B using commercially available transfection reagents. OTC protein expression levels were measured by near-infrared fluorescent imaging systems. Commercially available antibodies for OTC were used for detection. Untransfected and reference sequences were used as internal controls. FIG. 2, panel A is a scatter plot showing the correlation of OTC protein expression levels in Hepa1,6 cells at 24 hours as a function of mRNAs tested in N1MPU and 5MeOU chemistries. FIG. 2, panel B is a scatter plot showing the correlation of OTC protein expression levels in Hep3B cells at 24 hours as a function of mRNAs tested in N1MPU and 5MeOU chemistries. These figures exhibit the degree of variability in expression levels when mRNAs from two different chemistries are tested in a mouse and a human liver cell line. It can be seen that in this experiment, most of the compounds express better when an N1MPU chemistry is used in the mRNA.

### Example 4: Round 2 of Protein Stability Compounds Screened in Human Primary Hepatocytes at 24 h and 48 h—Correlation

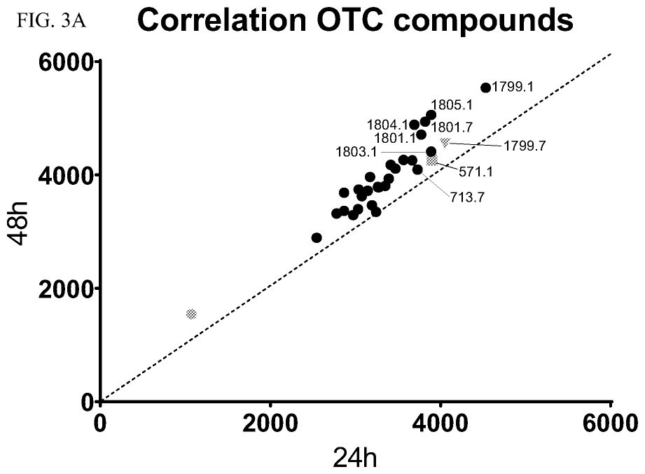

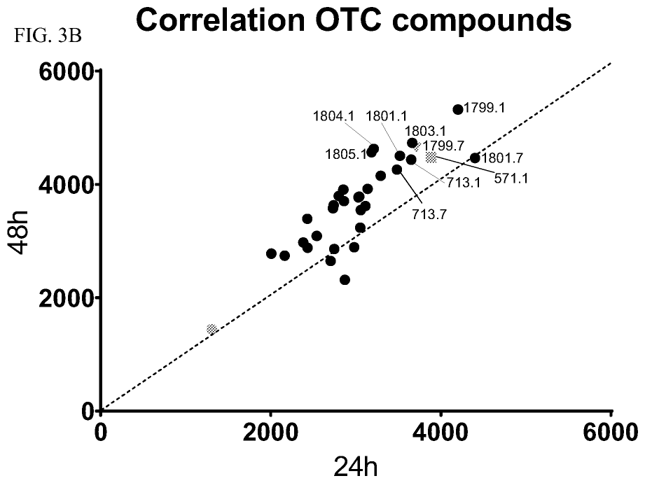

In vitro screening of certain mRNA constructs of Table 5 that were designed based on a protein stability approach was performed. The mRNAs were tested in two different chemistries, 100% of the Uridines are N1MPU indicated by the name of mRNA constructs followed by “0.1” and 100% of the uridines are 5MeOU indicated by the name of mRNA constructs followed by “0.7”. In-Cell Western (ICW) assays as described in Example 1 were used to transfect the different mRNAs into human primary hepatocytes using commercially available transfection reagents. OTC protein expression levels were measured by near-infrared fluorescent imaging systems. Commercially available antibodies for OTC were used for detection. Untransfected and reference sequences were used as internal controls. (FIG. 3, Panels A and B). The results indicate that, in contrast to the experiments conducted in cancer cell lines (Hepa1,6; Hep3B; Example 3, both chemistries express similarly in human primary hepatocytes.

### Example 5: Round 3 of Protein Stability Compounds Screening in Human Primary Hepatocytes at 24 h and 48 h—Correlation

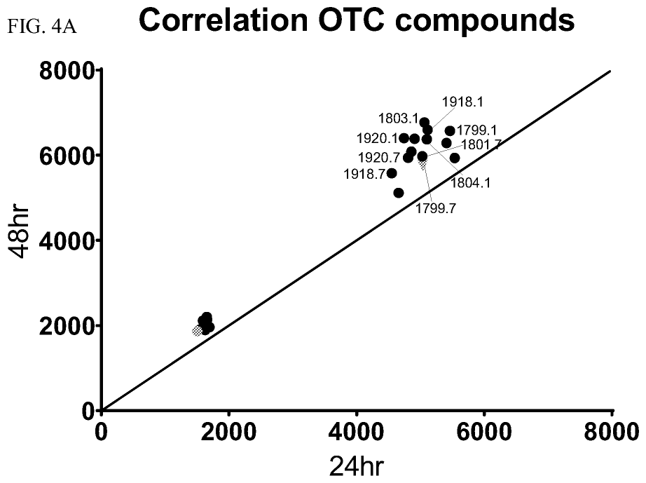

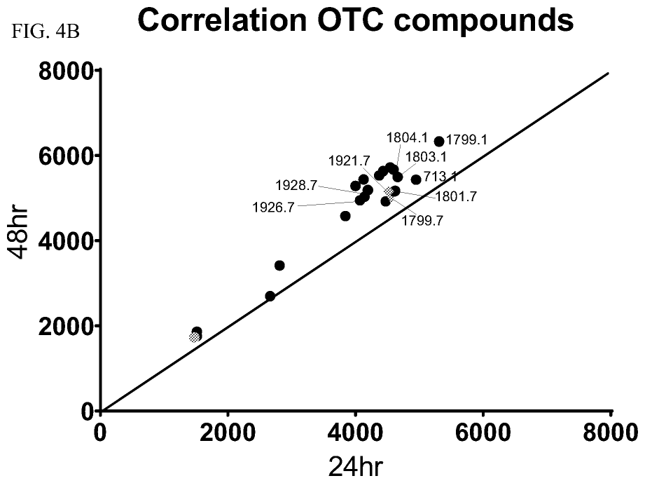

In vitro screening of novel compounds designed based on a protein stability approach was performed. mRNAs were tested in two different chemistries, N1MPU indicated by the name of mRNA constructs followed by “0.1” and 5MeOU indicated by the name of mRNA constructs followed by “0.7”. In-Cell Western (ICW) assays as described in Example 1 were used to transfect the different mRNAs into human primary hepatocytes using commercially-available transfection reagents. OTC protein expression levels were measured by near-infrared fluorescent imaging systems. Commercially available antibodies for OTC protein were used for detection. Untransfected and reference sequences were used as internal controls. (FIG. 4, Panels A and B). The results indicate that, in contrast to the experiments conducted in cancer cell lines (Hepa1,6; Hep3B; Example 3), both chemistries express similarly in human primary hepatocyte.

### Example 6: OTC Protein-Expression Levels in Human Primary Cells Transfected with OTC mRNA Constructs 1799.7 (5MeOU Chemistry) Encoding the OTC Protein of SEQ ID NO: 3 and 1921.7 (5MeOU Chemistry) Encoding the Modified OTC Protein of SEQ ID NO: 4

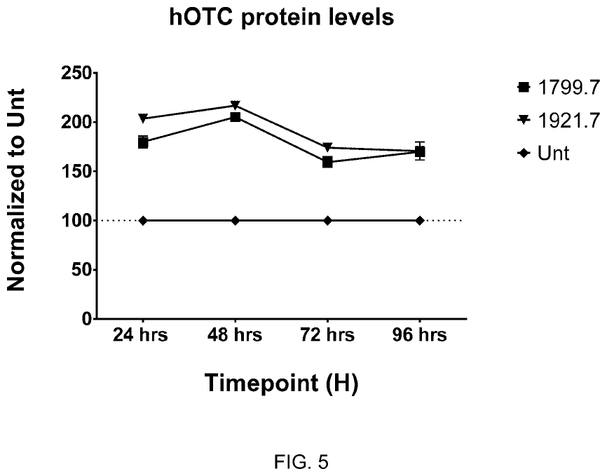

In-Cell Western (ICW) assays as described in Example 1 were used to transfect OTC mRNAs into human primary hepatocytes using commercially available transfection reagents. OTC protein expression levels were measured by near-infrared fluorescent imaging systems during a time course study up to 96 h. Commercially available OTC antibodies were used for detection. Untransfected cells were used as internal control. Plot shows OTC protein levels normalized to untransfected controls. (FIG. 5). The purpose of this study was to evaluate the half-life of the unmodified versus the modified protein sequence (1799.7, 1921.7) under in vitro conditions in transfected human primary hepatocytes. The results indicate that 1921.7 demonstrated more stable expression than 1799.7.

### Example 7: OTC Expression Levels Measured by Multiple Reaction Monitoring (MRM) Mass Spectrometry in Spf/Ash Mice Dosed at 10 mg/kg

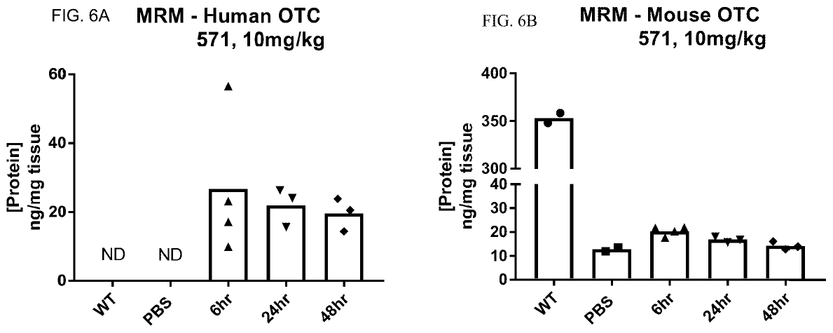

Spf/ash mice received an IV injection with either PBS or lipid-formulated OTC-mRNAs at a 10 mg/kg dosing. WT mice were used as internal controls to determine endogenous levels. A time course (6 h, 24 h and 48 h) was performed, and expression levels were measured by MRM using human and mouse specific epitopes for OTC. Graphs were made that represent the amount of protein (ng/mg tissue) detected by MRM specific for human OTC (FIG. 6, Panel A) or mouse (FIG. 6, Panel B). Human- and mouse-specific heavy peptides were designed to measure total levels of OTC in both species. This data set shows quantitative levels of human-specific OTC derived from translation of delivered mRNAs are detected. This expression is maintained a high level of stability up to 48 h.

### Example 8: OTC Expression Levels Measured by Western Blot in WT Mice Dosed at 3 mg/kg

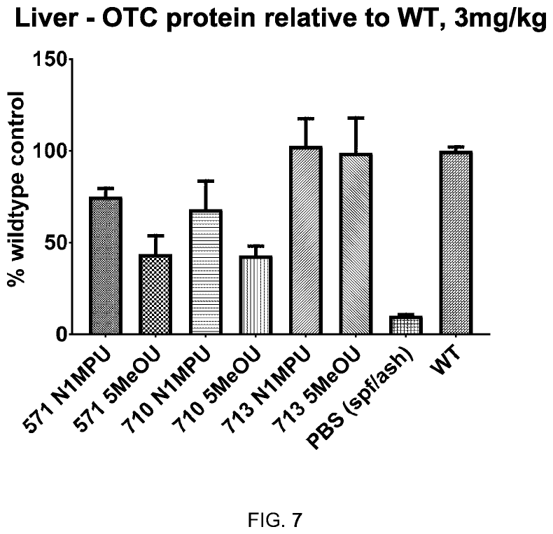

Spf/ash mice received an IV injection with either phosphate buffered saline (PBS) or lipid-formulated OTC-mRNAs at a 3 mg/kg dosing using two different chemistries (N1MPU and 5MeOU). WT mice were used as internal controls to determine endogenous levels. Animals were sacrificed 24 h post-dose. OTC expression levels were measured by Western Blot (WB) using an OTC specific antibody. In the results provided in FIG. 7, the bars represent the percentage of expression relative to WT levels (100%). The data generated in this figure shows that WT levels of total OTC in a mouse background were achieved for several codon-optimized sequences.

### Example 9: OTC Expression Levels Measured by MRM in a Dose Range Finding Study

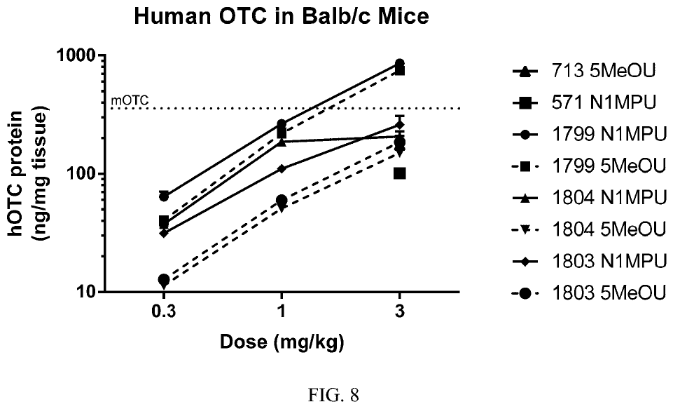

Balb/c mice received an IV injection with either PBS or lipid-formulated OTC-mRNAs at three different doses: 0.3 mg/kg, 1 mg/kg and 3 mg/kg and using two different chemistries (N1MPU and 5MeOU). Animals were sacrificed 24 h post-dose and expression levels were measured by MRM using human and mouse specific epitopes for OTC. The graph in FIG. 8 represents the percentage of expression of human OTC (ng) per mg of liver tissue in Balb/c mice. The horizontal dotted line represents the relative mouse OTC levels in Balb/c mice. (FIG. 8). Expression levels of hOTC protein for mRNA construct 713 5MeOU and mRNA construct 571 N1MPU are indicated by arrows in FIG. 8. In this figure, the MRM was used to quantitatively determine human-selective and mouse-selective OTC protein levels. The data generated in this figure shows, in a dose dependent manner, that WT levels of human OTC in a mouse background are achieved with the codon-optimized sequences disclosed herein.

### Example 10: Urinary Orotate Levels Measured in PBS and Treated Spf/Ash Mice

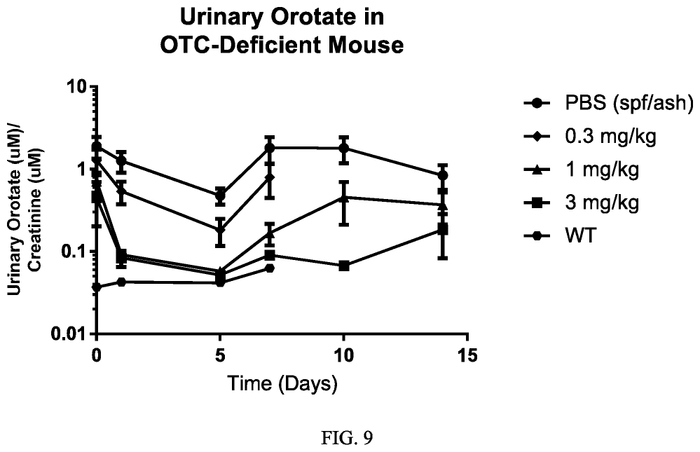

Spf/ash mice received an IV injection with either PBS or lipid-formulated OTC-mRNA construct 1799.7 (5MeOU chemistry) at three different doses: 0.3 mg/kg, 1 mg/kg and 3 mg/kg. WT and spf/ash mice were used to determine baseline and high urinary orotate levels, respectively. A Spf/ash time course was determined, and urinary orotate levels were measured at each timepoint. The results can be seen in FIG. 9. Urinary orotate was normalized to creatinine, which is represented in the graph in the Y axis throughout the time course and serve as a proof-of-concept of functional restoration of OTC activity post-injection. At 3 mg/kg, a sustainable reduction of urinary orotate levels up to 14 days was observed.

### Example 11: Pharmacokinetic/Pharmacodynamic (PK/PD) Analysis Comparing Human OTC Expression Levels and Urinary Orotate at 96 h

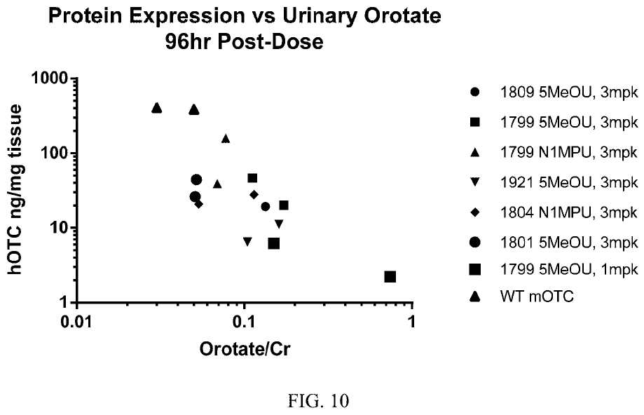

Spf/ash mice received an IV injection with either PBS or certain lipid-formulated OTC-mRNAs from Table 5 at 1 mg/kg and 3 mg/kg using two different chemistries (N1MPU and 5MeOU). WT mice were used as internal controls. Human-specific OTC levels were measured by MRM whereas urinary orotate was determined in each sample and normalized to creatinine. PK/PD is plotted in FIG. 10. PK/PD analysis shows the correlation between protein expression levels and reduction of urinary orotate in a compound-specific manner. Construct 1799.7 shows a high PK/PD correlation.

### Example 12: Fractioning of Spf/Ash Mice In Vivo Samples Treated with Selected mRNAs

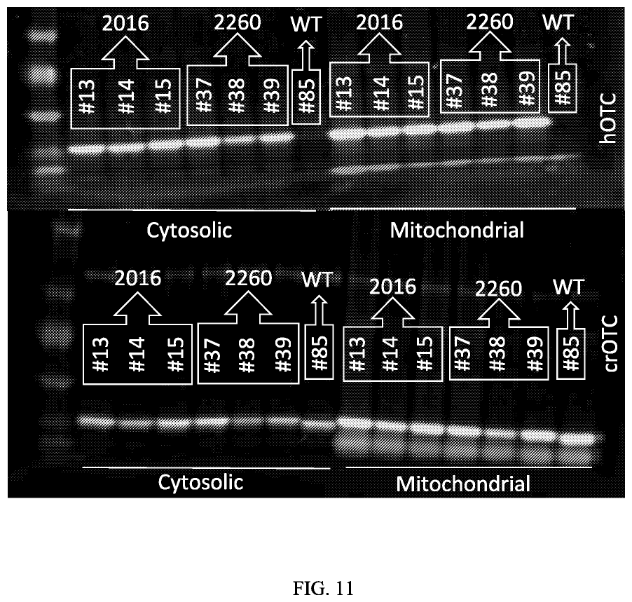

Spf/ash mice received an IV injection with either PBS or lipid-formulated OTC-mRNAs at 1 mg/kg and 3 mg/kg. WT mice were used as internal controls. Sample fractioning was performed on the liver samples, separating a cytosolic and a mitochondrial fraction. OTC levels were measured by WB using human specific (hOTC) and crossreactive (crOTC) antibodies (FIG. 11). Cyclooxygenase IV (CoxIV) was used as a mitochondrial control. OTC protein expression levels were measured by near-infrared fluorescent imaging systems and normalized to total protein. WB indicates differences in OTC expression levels within mitochondrial and cytosolic fractions when 2016 and 2260 mRNAs were delivered in the spf/ash mice. The 2260 mRNA encodes a protein with a modified signal peptide as described herein. These results indicate that both compounds can efficiently target the mitochondria.

### Example 13: Plot of the Mitochondrial Vs Cytosolic Fractions of Spf/Ash Mice Samples Treated with mRNA Constructs 2016 and 2260

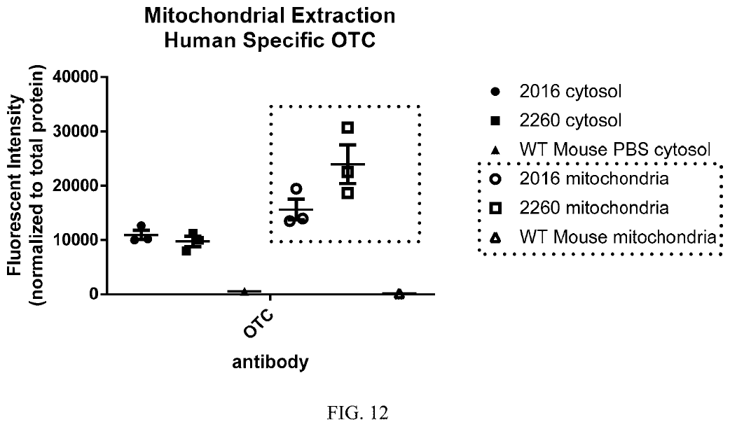

Spf/ash mice received an IV injection with either PBS or lipid-formulated OTC-mRNAs at 3 mg/kg. WT mice were used as internal controls. Sample fractioning was performed on the liver samples, separating a cytosolic and a mitochondrial fraction. OTC levels were measured by western blot using a human specific antibody. OTC protein expression levels were measured by near-infrared fluorescent imaging systems and both fractions normalized to total protein were plotted (FIG. 12). The plot of protein expression levels shown in FIG. 11 (Example 12), indicate that even though both compounds, 2016 and 2260, deliver similar protein levels in the cytosol, it is in the mitochondria where 2260 delivers more human OTC than 2016. The 2260 includes a modified mitochondrial signaling peptide sequence of the invention.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this disclosure has been particularly shown and described with references to preferred embodiments thereof, it will be understood by those skilled in the art that various changes in form and details may be made therein without departing from the scope of the disclosure encompassed by the appended claims. It should also be understood that the embodiments described herein are not mutually exclusive and that features from the various embodiments may be combined in whole or in part in accordance with the disclosure.

SEQUENCE LISTING

mRNA coding sequence for wild type human OTC
(SEQ ID NO: 1)
AUGCUGUUUAAUCUGAGGAUCCUGUUAAACAAUGCAGCUUUUAGAAAUGGU
CACAACUUCAUGGUUCGAAAUUUUCGGUGUGGACAACCACUACAAAAUAAAG
UGCAGCUGAAGGGCCGUGACCUUCUCACUCUAAAAAAC
UUUACCGGAGAAGAAAUUAAAUAUAUGCUAUGGCUAUCAGCAGAUCUGAAA
UUUAGGAUAAAACAGAAAGGAGAGUAUUUGCCUUUAUUGCAAGGGAAGUCC
UUAGGCAUGAUUUUUGAGAAAAGAAGUACUCGAACAAGAUUGUCUACAGAA
ACAGGCUUUGCACUUCUGGGAGGACAUCCUUGUUUUCUUACCACACAAGAUA
UUCAUUUGGGUGUGAAUGAAAGUCUCACGGACACGGCCCGUGUAUUGUCUAG
CAUGGCAGAUGCAGUAUUGGCUCGAGUGUAUAAACAAUCAGAUUUGGACACC
CUGGCUAAAGAAGCAUCCAUCCCAAUUAUCAAUGGGCUGUCAGAUUUGUACC
AUCCUAUCCAGAUCCUGGCUGAUUACCUCACGCUCCAGGAACACUAUAGCUC
UCUGAAAGGUCUUACCCUCAGCUGGAUCGGGGAUGGGAACAAUAUCCUGCAC
UCCAUCAUGAUGAGCGCAGCGAAAUUCGGAAUGCACCUUCAGGCAGCUACUC
CAAAGGGUUAUGAGCCGGAUGCUAGUGUAACCAAGUUGGCAGAGCAGUAUG
CCAAAGAGAAUGGUACCAAGCUGUUGCUGACAAAUGAUCCAUUGGAAGCAGC
GCAUGGAGGCAAUGUAUUAAUUACAGACACUUGGAUAAGCAUGGGACAAGA
AGAGGAGAAGAAAAAGCGGCUCCAGGCUUUCCAAGGUUACCAGGUUACAAUG
AAGACUGCUAAAGUUGCUGCCUCUGACUGGACAUUUUUACACUGCUUGCCCA
GAAAGCCAGAAGAAGUGGAUGAUGAAGUCUUUUAUUCUCCUCGAUCACUAG
UGUUCCCAGAGGCAGAAAACAGAAAGUGGACAAUCAUGGCUGUCAUGGUGUC
CCUGCUGACAGAUUACUCACCUCAGCUCCAGAAGCCUAAAUUUUGA
DNA coding sequence for wild type human OTC
(SEQ ID NO: 2)
ATGCTGTTTAATCTGAGGATCCTGTTAAACAATGCAGCTTTTAGAAATGGTCAC
AACTTCATGGTTCGAAATTTTCGGTGTGGACAACCACTACAAAATAAAGTGCAG
CTGAAGGGCCGTGACCTTCTCACTCTAAAAAACTTTACCGGAGAAGAAATTAAA
TATATGCTATGGCTATCAGCAGATCTGAAATTTAGGATAAAACAGAAAGGAGA
GTATTTGCCTTTATTGCAAGGGAAGTCCTTAGGCATGATTTTTGAGAAAAGAAG
TACTCGAACAAGATTGTCTACAGAAACAGGCTTTGCACTTCTGGGAGGACATCC
TTGTTTTCTTACCACACAAGATATTCATTTGGGTGTGAATGAAAGTCTCACGGA
CACGGCCCGTGTATTGTCTAGCATGGCAGATGCAGTATTGGCTCGAGTGTATAA
ACAATCAGATTTGGACACCCTGGCTAAAGAAGCATCCATCCCAATTATCAATGG
GCTGTCAGATTTGTACCATCCTATCCAGATCCTGGCTGATTACCTCACGCTCCAG
GAACACTATAGCTCTCTGAAAGGTCTTACCCTCAGCTGGATCGGGGATGGGAAC
AATATCCTGCACTCCATCATGATGAGCGCAGCGAAATTCGGAATGCACCTTCAG
GCAGCTACTCCAAAGGGTTATGAGCCGGATGCTAGTGTAACCAAGTTGGCAGA
GCAGTATGCCAAAGAGAATGGTACCAAGCTGTTGCTGACAAATGATCCATTGG
AAGCAGCGCATGGAGGCAATGTATTAATTACAGACACTTGGATAAGCATGGGA
CAAGAAGAGGAGAAGAAAAAGCGGCTCCAGGCTTTCCAAGGTTACCAGGTTAC
AATGAAGACTGCTAAAGTTGCTGCCTCTGACTGGACATTTTTACACTGCTTGCC
CAGAAAGCCAGAAGAAGTGGATGATGAAGTCTTTTATTCTCCTCGATCACTAGT
GTTCCCAGAGGCAGAAAACAGAAAGTGGACAATCATGGCTGTCATGGTGTCCC
TGCTGACAGATTACTCACCTCAGCTCCAGAAGCCTAAATTTTGA
Human wild type OTC amino acid sequence
(The signal peptide for mitochondrial import is underlined*)
(SEQ ID NO: 3)
MLFNLRILLNNAAFRNGHNFMVRNFRCGQPLQNKVQLKGRDLLTLKNFTGEEI
KYMLWLSADLKFRIKQKGEYLPLLQGKSLGMIFEKRSTRTRLSTETGFALLGGHPC
FLTTQDIHLGVNESLTDTARVLSSMADAVLARVYKQSDLDTLAKEASIPIF
GLSDLYHPIQILADYLTLQEHYSSLKGLTLSWIGDGNNILHSIMMSAAKFGMHLQA
ATPKGYEPDASVTKLAEQYAKENGTKLLLTNDPLEAAHGGNVLITDTWISMGQEE
EKKKRLQAFQGYQVTMKTAKVAASDWTFLHCLPRKPEEVDDEVFYSPRSLVFPEA
ENRKWTIMAVMVSLLTDYSPQLQKPKF
Modified OTC amino acid sequence
(The signal peptide for mitochondrial import is underlined*)
(SEQ ID NO: 4)
MLVFNLRILLNNAAFRNGHNFMVRNFRCGQPLQNRVQLKGRDLLTLKNFTGEEI
RYMLWLSADLKFRIKQKGEYLPLLQGKSLGMIFEKRSTRTRLSTETGFALLGGHPC
FLTTQDIHLGVNESLTDTARVLSSMADAVLARVYKQSDLDTLAKEASIPIINGLSDL
YHPIQILADYLTLQEHYSSLKGLTLSWIGDGNNILHSIMMSAAKFGMHLQAATPKG
YEPDASVTKLAEQYAKENGTKLLLTNDPLEAAHGGNVLITDTWISMGQEEEKKKR
LQAFQGYQVTMKTAKVAASDWTFLHCLPRKPEEVDDEVFYSPRSLVFPEAENRKW
TIMAVMVSLLTDYSPQLQKPKF
TEV
(SEQ ID NO: 5)
TCAACACAACATATACAAAACAAACGAATCTCAAGCAATCAAGCATTCTACTTC
TATTGCAGCAATTTAAATCATTTCTTTTAAAGCAAAAGCAATTTTCTGAAAATTT
TCACCATTTACGAACGATAG
AT1G58420
(SEQ ID NO: 6)
ATTATTACATCAAAACAAAAAGCCGCCA
ARC5-2
(SEQ ID NO: 7)
CTTAAGGGGGCGCTGCCTACGGAGGTGGCAGCCATCTCCTTCTCGGCATCAAGC
TTACCATGGTGCCCCAGGCCCTGCTCTTgGTCCCGCTGCTGGTGTTCCCCCTCTG
CTTCGGCAAGTTCCCCATCTACACCATCCCCGACAAGCTGGGGCCGTGGAGCCC
CATCGACATCCACCACCTGTCCTGCCCCAACAACCTCGTGGTCGAGGACGAGGG
CTGCACCAACCTGAGCGGGTTCTCCTAC
HCV
(SEQ ID NO: 8)
TGAGTGTCGT ACAGCCTCCA GGCCCCCCCC TCCCGGGAGA GCCATAGTGG
TCTGCGGAACCGGTGAGTAC ACCGGAATTG CCGGGAAGAC TGGGTCCTTT
CTTGGATAAA CCCACTCTATGCCCGGCCAT TTGGGCGTGC CCCCGCAAGA
CTGCTAGCCG AGTAGTGTTG GGTTGCG
HUMAN ALBUMIN
(SEQ ID NO: 9)
AATTATTGGTTAAAGAAGTATATTAGTGCTAATTTCCCTCCGTTTGTCCTAGCTT
TTCTCTTCTGTCAACCCCACACGCCTTTGGCACA
EMCV
(SEQ ID NO: 10)
CTCCCTCCCC CCCCCCTAAC GTTACTGGCC GAAGCCGCTT GGAATAAGGC
CGGTGTGCGT TTGTCTATAT GTTATTTTCC ACCATATTGC CGTCTTTTGG
CAATGTGAGG GCCCGGAAAC CTGGCCCTGT CTTCTTGACG AGCATTCCTA
GGGGTCTTTC CCCTCTCGCC AAAGGAATGC AAGGTCTGTT GAATGTCGTG
AAGGAAGCAG TTCCTCTGGA AGCTTCTTGA AGACAAACAA CGTCTGTAGC
GACCCTTTGC AGGCAGCGGA ACCCCCCACC TGGCGACAGG TGCCTCTGCG
GCCAAAAGCC ACGTGTATAA GATACACCTG CAAAGGCGGC ACAACCCCAG
TGCCACGTTG TGAGTTGGAT AGTTGTGGAA AGAGTCAAAT GGCTCTCCTC
AAGCGTATTC AACAAGGGGC TGAAGGATGC CCAGAAGGTA CCCCATTGTA
TGGGATCTGA TCTGGGGCCT CGGTGCACAT GCTTTACGTG TGTTTAGTCG
AGGTTAAAAA ACGTCTAGGC CCCCCGAACC ACGGGGACGT GGTTTTCCTT
TGAAAAACAC GATGATAAT
HSP70-P2
(SEQ ID NO: 11)
GTCAGCTTTCAAACTCTTTGTTTCTTGTTTGTTGATTGAGAATA
HSP70-M1
(SEQ ID NO: 12)
CTCTCGCCTGAGAAAAAAAATCCACGAACCAATTTCTCAGCAACCAGCAGCAC
G
HSP72-M2
(SEQ ID NO: 13)
ACCTGTGAGGGTTCGAAGGAAGTAGCAGTGTTTTTTGTTCCTAGAGGAAGAG
HSP17.9
(SEQ ID NO: 14)
ACACAGAAACATTCGCAAAAACAAAATCCCAGTATCAAAATTCTTCTCTTTTTT
TCATATTTCGCAAAGAC
HSP70-P1
(SEQ ID NO: 15)
CAGAAAAATTTGCTACATTGTTTCACAAACTTCAAATATTATTCATTTATTT
XBG
(SEQ ID NO: 16)
CTAGTGACTGACTAGGATCTGGTTACCACTAAACCAGCCTCAAGAACACCCGAA
TGGAGTCTCTAAGCTACATAATACCAACTTACACTTACAAAATGTTGTCCCCCA
AAATGTAGCCATTCGTATCTGCTCCTAATAAAAAGAAAGTTTCTTCACAT
HUMAN HAPTOGLOBIN
(SEQ ID NO: 17)
TGCAAGGCTGGCCGGAAGCCCTTGCCTGAAAGCAAGATTTCAGCCTGGAAGAG
GGCAAAGTGGACGGGAGTGGACAGGAGTGGATGCGATAAGATGTGGTTTGAAG
CTGATGGGTGCCAGCCCTGCATTGCTGAGTCAATCAATAAAGAGCTTTCTTTTG
ACCCAT
HUMAN APOLIPOPROTEIN E
(SEQ ID NO: 18)
ACGCCGAAGCCTGCAGCCATGCGACCCCACGCCACCCCGTGCCTCCTGCCTCCG
CGCAGCCTGCAGCGGGAGACCCTGTCCCCGCCCCAGCCGTCCTCCTGGGGTGGA
CCCTAGTTTAATAAAGATTCACCAAGTTTCACGCA
HCV
(SEQ ID NO: 19)
TAGAGCGGCAAACCCTAGCTACACTCCATAGCTAGTTTCTTTTTTTTTTGTTTTTT
TTTTTTTTTTTTTTTTTTTTTTTTTTTTTTTTTCCTTTCTTTTCCTTCTTTTTTTCCTC
TTTTCTTGGTGGCTCCATCTTAGCCCTAGTCACGGCTAGCTGTGAAAGGTCCGTG
AGCCGCATGACTGCAGAGAGTGCCGTAACTGGTCTCTCTGCAGATCATGT
MOUSE ALBUMIN
(SEQ ID NO: 20)
ACACATCACAACCACAACCTTCTCAGGCTACCCTGAGAAAAAAAGACATGAAG
ACTCAGGACTCATCTTTTCTGTTGGTGTAAAATCAACACCCTAAGGAACACAAA
TTTCTTTAAACATTTGACTTCTTGTCTCTGTGCTGCAATTAATAAAAAATGGAAA
GAATCTAC
HUMAN ALPHA GLOBIN
(SEQ ID NO: 21)
GCTGGAGCCTCGGTAGCCGTTCCTCCTGCCCGCTGGGCCTCCCAACGGGCCCTC
CTCCCCTCCTTGCACCGGCCCTTCCTGGTCTTTGAATAAAGTCTGAGTGGGCAGC
A
EMCV
(SEQ ID NO: 22)
TAGTGCAGTCAC TGGCACAACG CGTTGCCCGG TAAGCCAATC GGGTATACAC
GGTCGTCATACTGCAGACAG GGTTCTTCTA CTTTGCAAGA TAGTCTAGAG
TAGTAAAATA AATAGTATAAG
(SEQ ID NO: 23)
GCCACC
(SEQ ID NO: 24)
GCCA
(SEQ ID NO: 25)
AUAAGUGAA
>mARM563
(SEQ ID NO: 26)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUGUUUAAUCUGAGGAUC
CUGUUAAACAAUGCAGCUUUUAGAAAUGGUCACAACUUCAUGGUUCGAAAU
UUUCGGUGUGGACAACCACUACAAAAUAAAGUGCAGCUGAAGGGCCGUGACC
UUCUCACUCUAAAAAACUUUACCGGAGAAGAAAUUAAAUAUAUGCUAUGGC
UAUCAGCAGAUCUGAAAUUUAGGAUAAAACAGAAAGGAGAGUAUUUGCCUU
UAUUGCAAGGGAAGUCCUUAGGCAUGAUUUUUGAGAAAAGAAGUACUCGAA
CAAGAUUGUCUACAGAAACAGGCUUUGCACUUCUGGGAGGACAUCCUUGUUU
UCUUACCACACAAGAUAUUCAUUUGGGUGUGAAUGAAAGUCUCACGGACACG
GCCCGUGUAUUGUCUAGCAUGGCAGAUGCAGUAUUGGCUCGAGUGUAUAAAC
AAUCAGAUUUGGACACCCUGGCUAAAGAAGCAUCCAUCCCAAUUAUCAAUGG
GCUGUCAGAUUUGUACCAUCCUAUCCAGAUCCUGGCUGAUUACCUCACGCUC
CAGGAACACUAUAGCUCUCUGAAAGGUCUUACCCUCAGCUGGAUCGGGGAUG
GGAACAAUAUCCUGCACUCCAUCAUGAUGAGCGCAGCGAAAUUCGGAAUGCA
CCUUCAGGCAGCUACUCCAAAGGGUUAUGAGCCGGAUGCUAGUGUAACCAAG
UUGGCAGAGCAGUAUGCCAAAGAGAAUGGUACCAAGCUGUUGCUGACAAAU
GAUCCAUUGGAAGCAGCGCAUGGAGGCAAUGUAUUAAUUACAGACACUUGG
AUAAGCAUGGGACAAGAAGAGGAGAAGAAAAAGCGGCUCCAGGCUUUCCAA
GGUUACCAGGUUACAAUGAAGACUGCUAAAGUUGCUGCCUCUGACUGGACAU
UUUUACACUGCUUGCCCAGAAAGCCAGAAGAAGUGGAUGAUGAAGUCUUUU
AUUCUCCUCGAUCACUAGUGUUCCCAGAGGCAGAAAACAGAAAGUGGACAAU
CAUGGCUGUCAUGGUGUCCCUGCUGACAGAUUACUCACCUCAGCUCCAGAAG
CCUAAAUUUUGACUCGAGCUAGUGACUGACUAGGAUCUGGUUACCACUAAAC
CAGCCUCAAGAACACCCGAAUGGAGUCUCUAAGCUACAUAAUACCAACUUAC
ACUUACAAAAUGUUGUCCCCCAAAAUGUAGCCAUUCGUAUCUGCUCCUAAUA
AAAAGAAAGUUUCUUCACAUUCUAG
>mARM564
(SEQ ID NO: 27)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUCUUUAAUCUGCGCAUC
UUACUGAACAACGCCGCAUUCCGGAACGGUCACAACUUCAUGGUCCGCAAUU
UCCGCUGUGGCCAGCCGCUUCAAAACAAGGUCCAGCUGAAGGGACGGGAUCU
GCUGACACUGAAGAACUUCACCGGAGAAGAGAUCAAGUACAUGCUGUGGCUC
AGCGCAGACUUGAAGUUCCGGAUCAAGCAGAAGGGAGAAUACUUGCCCCUGC
UGCAAGGAAAGUCGCUGGGAAUGAUUUUUGAGAAGCGGUCAACUCGCACCAG
ACUCUCCACCGAAACUGGUUUCGCACUGCUUGGCGGGCACCCUUGCUUCCUG
ACGACUCAGGACAUCCACCUCGGCGUGAACGAAUCGCUAACCGAUACCGCCA
GAGUGCUUUCUUCCAUGGCCGACGCGGUGCUGGCCAGGGUGUACAAGCAGUC
CGACCUCGAUACCUUGGCAAAGGAGGCUUCCAUUCCCAUCAUCAACGGCCUG
AGCGACCUGUACCACCCAAUCCAAAUCCUGGCUGACUACCUGACCCUGCAAG
AGCACUACAGCAGCCUGAAGGGUCUGACCCUGUCAUGGAUUGGCGAUGGAAA
CAAUAUUCUGCACUCCAUCAUGAUGUCCGCCGCGAAGUUCGGAAUGCAUCUG
CAAGCCGCCACUCCAAAAGGAUACGAACCGGAUGCGUCCGUGACCAAGUUGG
CGGAACAGUACGCGAAGGAGAACGGAACCAAGCUUCUGCUGACUAACGACCC
CCUCGAGGCUGCGCAUGGGGGCAACGUGCUGAUUACCGACACCUGGAUCUCC
AUGGGGCAGGAGGAAGAGAAGAAGAAGAGACUGCAGGCAUUCCAGGGGUAC
CAGGUCACCAUGAAAACCGCAAAAGUGGCAGCUUCGGACUGGACUUUCCUGC
AUUGCCUGCCGAGGAAGCCGGAGGAAGUCGACGACGAAGUGUUCUACUCGCC
UCGGUCCCUGGUGUUCCCCGAGGCCGAAAACCGGAAGUGGACCAUCAUGGCC
GUGAUGGUGUCCUUGCUGACUGACUAUAGCCCGCAGCUGCAGAAGCCUAAGU
UCUAGCUCGAGCUAGUGACUGACUAGGAUCUGGUUACCACUAAACCAGCCUC
AAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAA
AGUUUCUUCACAUUCUAG
>mARM565
(SEQ ID NO: 28)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUGUUUAACCUACGUAUU
UUGCUCAACAAUGCAGCCUUUAGAAACGGACAUAACUUUAUGGUUCGAAACU
UUCGCUGCGGGCAGCCACUGCAGAACAAGGUCCAGCUGAAAGGGAGAGAUUU
GCUCACGCUGAAGAACUUUACUGGCGAAGAAAUCAAGUAUAUGCUGUGGUU
GUCCGCGGACCUCAAGUUUCGGAUUAAGCAGAAAGGGGAGUAUCUGCCACUG
CUGCAAGGAAAGAGCCUCGGCAUGAUCUUCGAGAAGCGGAGCACUCGGACCA
GGCUGAGUACCGAAACUGGCUUCGCAUUGUUGGGUGGACAUCCAUGUUUUCU
GACAACGCAGGACAUUCAUCUGGGCGUGAACGAGAGUCUGACGGACACAGCU
CGCGUUCUGUCCUCUAUGGCUGAUGCGGUGUUGGCCCGGGUCUAUAAGCAGU
CCGAUUUGGACACCUUGGCUAAGGAAGCUAGCAUACCGAUUAUCAAUGGGCU
GUCCGACCUGUAUCACCCUAUUCAAAUCCUGGCCGACUACCUCACACUGCAA
GAACACUAUAGCUCAUUGAAGGGACUGACCCUGAGCUGGAUAGGGGACGGAA
ACAACAUCCUACAUAGCAUUAUGAUGUCCGCUGCCAAGUUUGGCAUGCAUCU
UCAAGCCGCCACGCCAAAGGGUUAUGAGCCCGACGCGUCAGUGACAAAGCUG
GCCGAGCAGUACGCUAAGGAGAAUGGUACCAAAUUACUGCUGACUAAUGAUC
CACUGGAGGCUGCACAUGGCGGCAAUGUACUGAUCACCGACACAUGGAUCUC
GAUGGGCCAGGAGGAAGAAAAGAAGAAGAGGCUUCAGGCCUUCCAAGGCUAC
CAGGUCACCAUGAAAACAGCUAAGGUUGCAGCAUCUGAUUGGACCUUUCUGC
ACUGUCUGCCAAGGAAGCCCGAAGAGGUGGACGAUGAAGUAUUCUAUAGCCC
ACGGAGUUUGGUGUUCCCUGAGGCUGAAAAUAGGAAGUGGACAAUUAUGGC
CGUAAUGGUGUCCCUGUUAACCGACUACUCUCCGCAACUGCAGAAACCUAAG
UUUUAGCUCGAGCUAGUGACUGACUAGGAUCUGGUUACCACUAAACCAGCCU
CAAGAACACCCGAAUGGAGUCUCUAAGCUACAUAAUACCAACUUACACUUAC
AAAAUGUUGUCCCCCAAAAUGUAGCCAUUCGUAUCUGCUCCUAAUAAAAAGA
AAGUUUCUUCACAUUCUAG
>mARM566
(SEQ ID NO: 29)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUGUUUAACUUAAGGAUC
CUGCUGAACAACGCCGCUUUUCGUAACGGUCAUAACUUUAUGGUCCGGAACU
UUAGAUGUGGCCAGCCGCUGCAGAACAAGGUUCAGCUGAAGGGGAGGGAUCU
GCUGACCUUGAAGAACUUUACCGGCGAAGAGAUCAAGUACAUGUUGUGGCUG
AGCGCCGAUCUGAAGUUUAGGAUUAAGCAGAAGGGGGAGUAUUUGCCACUG
CUGCAAGGAAAAUCCUUGGGGAUGAUCUUCGAGAAGCGCUCCACUAGAACCC
GGCUAAGCACAGAAACCGGCUUCGCACUUCUGGGUGGACAUCCCUGUUUUCU
GACGACGCAGGAUAUACACCUGGGCGUGAAUGAGAGUCUGACGGACACAGCU
AGGGUGUUGAGCAGCAUGGCCGAUGCAGUACUGGCCCGCGUUUAUAAGCAGA
GCGACUUGGACACACUGGCCAAGGAAGCGUCAAUUCCGAUUAUCAAUGGGCU
GUCAGACCUGUAUCAUCCCAUUCAAAUCUUGGCUGACUAUCUGACCCUGCAA
GAACAUUACAGCUCCCUGAAGGGCCUCACGUUGUCCUGGAUUGGCGACGGAA
ACAACAUUCUGCAUUCGAUCAUGAUGAGCGCUGCUAAGUUUGGCAUGCACCU
CCAAGCCGCUACACCUAAGGGAUAUGAGCCUGAUGCCAGCGUAACCAAGCUG
GCCGAACAGUACGCGAAGGAGAAUGGCACGAAACUGCUGUUGACAAAUGACC
CACUGGAGGCAGCUCACGGUGGCAACGUGCUGAUCACCGACACGUGGAUAUC
UAUGGGACAGGAAGAAGAGAAGAAGAAGCGGCUGCAGGCAUUCCAAGGGUA
UCAGGUCACCAUGAAAACGGCCAAGGUUGCUGCAUCCGACUGGACAUUUCUG
CAUUGCUUGCCCCGCAAACCAGAAGAAGUAGACGACGAAGUCUUUUAUUCCC
CACGGUCGCUGGUGUUCCCCGAGGCGGAGAAUCGAAAGUGGACGAUUAUGGC
CGUGAUGGUGUCCCUGCUGACUGAUUACUCUCCCCAACUGCAAAAGCCUAAG
UUUUAGCUCGAGCUAGUGACUGACUAGGAUCUGGUUACCACUAAACCAGCCU
CAAGAACACCCGAAUGGAGUCUCUAAGCUACAUAAUACCAACUUACACUUAC
AAAAUGUUGUCCCCCAAAAUGUAGCCAUUCGUAUCUGCUCCUAAUAAAAAGA
AAGUUUCUUCACAUUCUAG
>mARM567
(SEQ ID NO: 30)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAACCUGAGGAUC
CUCCUGAACAACGCCGCCUUUCGCAAUGGUCACAACUUUAUGGUCCGGAACU
UCAGAUGCGGCCAGCCGCUGCAGAACAAGGUCCAGCUGAAGGGACGGGAUCU
GCUGACUCUGAAGAACUUCACCGGAGAAGAGAUCAAGUACAUGCUGUGGCUG
UCGGCCGACCUGAAGUUCAGGAUCAAGCAGAAGGGAGAAUACCUCCCGCUGC
UGCAAGGAAAGUCCCUGGGCAUGAUUUUCGAGAAGCGCUCGACCAGAACUCG
GUUGUCCACCGAAACCGGGUUUGCGCUGCUGGGCGGACAUCCUUGCUUCCUG
ACGACUCAGGAUAUUCACCUGGGAGUGAACGAGUCGCUGACCGACACCGCCA
GAGUGCUGAGCUCGAUGGCCGACGCCGUGUUGGCACGCGUGUACAAGCAGUC
CGAUCUGGAUACCCUGGCCAAAGAAGCUUCCAUCCCGAUCAUUAACGGGCUG
AGCGACCUCUACCACCCCAUUCAAAUCCUGGCCGACUACCUGACUCUGCAAG
AACACUACAGCUCGCUGAAGGGGUUGACUCUGUCCUGGAUCGGCGACGGAAA
CAACAUCCUGCACUCCAUCAUGAUGUCGGCCGCAAAGUUCGGCAUGCAUUUG
CAAGCCGCCACCCCAAAGGGCUACGAACCAGACGCGAGCGUCACCAAGCUGG
CCGAACAGUACGCGAAGGAAAAUGGUACUAAGCUGCUGCUGACCAACGACCC
AUUGGAAGCUGCCCAUGGUGGAAACGUGCUGAUCACCGACACCUGGAUCUCG
AUGGGCCAGGAAGAGGAGAAGAAGAAGCGGCUGCAGGCGUUCCAGGGGUAU
CAGGUCACCAUGAAAACAGCCAAAGUGGCAGCGUCAGACUGGACCUUCCUCC
ACUGUCUGCCUCGCAAGCCAGAGGAGGUGGACGACGAGGUGUUCUACUCCCC
UCGGUCCCUCGUGUUCCCUGAGGCUGAGAACCGGAAGUGGACCAUUAUGGCC
GUGAUGGUGUCACUCCUGACUGAUUACUCCCCGCAACUGCAGAAGCCCAAGU
UCUAGCUCGAGCUAGUGACUGACUAGGAUCUGGUUACCACUAAACCAGCCUC
AAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAA
AGUUUCUUCACAUUCUAG
>mARM568
(SEQ ID NO: 31)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUGUUUAACCUGAGGAUC
CUAUUGAACAAUGCUGCUUUUCGUAAUGGCCAUAACUUUAUGGUUCGGAACU
UUAGAUGCGGGCAGCCACUGCAGAACAAGGUCCAGUUGAAAGGCCGCGAUCU
GUUGACAUUGAAGAACUUUACCGGCGAAGAGAUUAAGUAUAUGCUGUGGCU
GUCUGCUGACCUCAAGUUUCGAAUCAAGCAGAAGGGCGAAUAUCUCCCCCUG
CUGCAAGGAAAGUCUCUCGGCAUGAUCUUUGAGAAGCGGAGUACCCGAACAC
GGCUGAGCACCGAAACGGGCUUCGCACUGCUGGGGGGCCAUCCCUGUUUUCU
GACAACGCAGGACAUCCACUUGGGGGUUAACGAAUCAUUGACUGAUACCGCC
CGCGUACUGUCAUCCAUGGCCGACGCUGUGCUGGCUAGGGUGUACAAGCAGU
CAGAUCUGGAUACACUGGCCAAGGAAGCUAGCAUACCAAUCAUCAAUGGACU
GAGUGACCUUUAUCACCCGAUUCAAAUACUAGCCGAUUAUCUGACCCUGCAA
GAGCAUUACUCCUCGCUGAAAGGCCUCACGCUGUCCUGGAUCGGCGACGGCA
ACAACAUUCUGCAUAGUAUUAUGAUGUCUGCUGCCAAAUUCGGCAUGCAUCU
GCAAGCUGCUACGCCGAAGGGUUAUGAACCCGACGCGUCAGUUACGAAGCUC
GCUGAGCAGUAUGCAAAGGAGAAUGGCACAAAGCUGUUGCUUACCAACGAUC
CCCUGGAAGCUGCUCAUGGCGGCAAUGUGCUGAUUACUGACACCUGGAUUUC
AAUGGGCCAGGAGGAGGAGAAGAAGAAGAGGUUACAGGCUUUUCAAGGUUA
CCAAGUCACGAUGAAAACCGCUAAGGUCGCAGCCAGCGACUGGACAUUCCUG
CACUGUCUGCCAAGAAAGCCGGAAGAAGUGGACGACGAGGUGUUCUAUUCCC
CGCGGUCUUUGGUGUUUCCGGAGGCCGAAAACAGGAAAUGGACCAUUAUGGC
CGUGAUGGUAUCGUUGCUGACGGACUACAGCCCUCAGUUGCAAAAGCCCAAG
UUCUAGCUCGAGCUAGUGACUGACUAGGAUCUGGUUACCACUAAACCAGCCU
CAAGAACACCCGAAUGGAGUCUCUAAGCUACAUAAUACCAACUUACACUUAC
AAAAUGUUGUCCCCCAAAAUGUAGCCAUUCGUAUCUGCUCCUAAUAAAAAGA
AAGUUUCUUCACAUUCUAG
>mARM569
(SEQ ID NO: 32)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUCUUUAACCUCCGCAUC
CUCCUCAACAACGCCGCCUUCCGGAAUGGGCAUAACUUCAUGGUCCGGAACU
UCAGAUGCGGCCAGCCCCUGCAAAACAAGGUCCAGUUGAAGGGACGGGACCU
CCUUACGCUGAAGAACUUUACCGGAGAAGAGAUUAAGUACAUGCUGUGGUU
GUCCGCUGACCUCAAGUUCCGCAUUAAGCAGAAGGGAGAAUAUCUGCCGCUG
CUGCAAGGAAAGAGCCUGGGCAUGAUCUUCGAAAAGCGCUCCACUAGAACCC
GGCUGUCGACUGAGACUGGAUUCGCCUUGCUCGGUGGACACCCGUGCUUCCU
GACGACCCAGGACAUCCACCUGGGAGUGAACGAGUCACUUACGGAUACCGCG
AGGGUGCUGUCCUCAAUGGCCGACGCAGUGCUCGCGCGCGUGUACAAGCAGU
CAGAUCUGGAUACCCUGGCCAAGGAAGCCAGCAUUCCCAUCAUCAACGGACU
GAGCGACCUUUACCACCCAAUCCAGAUCCUCGCCGACUACUUAACCCUGCAA
GAGCACUACAGCUCCCUGAAGGGACUGACUCUGUCCUGGAUCGGGGAUGGAA
ACAACAUCCUGCACUCCAUCAUGAUGUCUGCCGCUAAGUUUGGGAUGCAUCU
GCAAGCCGCAACCCCUAAGGGAUACGAGCCCGACGCCUCGGUGACCAAACUU
GCGGAACAGUACGCCAAGGAAAACGGUACCAAGCUGCUGCUGACCAACGACC
CUCUGGAAGCGGCCCACGGAGGAAAUGUGCUGAUUACCGACACCUGGAUUUC
GAUGGGCCAGGAGGAGGAGAAGAAGAAGAGACUGCAGGCGUUCCAGGGAUA
UCAGGUCACCAUGAAAACCGCCAAGGUCGCCGCCAGCGACUGGACCUUCCUG
CACUGUCUCCCUCGGAAACCGGAAGAAGUGGAUGACGAGGUGUUCUACUCCC
CGCGCUCGCUGGUGUUCCCGGAGGCUGAAAACAGGAAGUGGACAAUCAUGGC
CGUGAUGGUGUCCCUGUUGACCGACUACUCCCCACAACUGCAGAAGCCCAAG
UUCUAGCUCGAGCUAGUGACUGACUAGGAUCUGGUUACCACUAAACCAGCCU
CAAGAACACCCGAAUGGAGUCUCUAAGCUACAUAAUACCAACUUACACUUAC
AAAAUGUUGUCCCCCAAAAUGUAGCCAUUCGUAUCUGCUCCUAAUAAAAAGA
AAGUUUCUUCACAUUCUAG
>mARM570
(SEQ ID NO: 33)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAAUCUGCGCAUC
CUCCUGAACAACGCCGCCUUCCGCAAUGGACACAACUUUAUGGUCCGCAACU
UCCGCUGUGGGCAGCCGCUGCAGAACAAGGUCCAGCUCAAGGGGAGAGAUCU
CCUGACCCUGAAGAACUUCACUGGAGAGGAGAUCAAGUACAUGCUGUGGCUG
UCCGCCGACCUGAAAUUUCGGAUUAAGCAGAAGGGCGAAUACCUCCCACUGC
UGCAAGGAAAGUCUUUGGGCAUGAUCUUCGAAAAGAGAAGCACCCGGACCCG
GUUGAGCACCGAAACUGGGUUCGCGCUCCUCGGUGGACACCCGUGCUUCCUG
ACCACCCAAGAUAUUCAUCUGGGUGUCAACGAAAGCCUGACCGACACCGCCA
GGGUGCUGUCAUCCAUGGCUGACGCAGUGCUCGCCCGGGUGUACAAGCAGUC
AGACCUGGACACCCUCGCCAAGGAAGCUUCGAUCCCUAUCAUCAACGGACUU
UCCGACCUGUACCACCCCAUCCAAAUUCUGGCCGACUACCUGACUCUGCAAG
AACACUAUAGCUCGCUGAAAGGACUUACUCUGUCCUGGAUCGGGGACGGCAA
CAACAUUCUCCAUUCCAUCAUGAUGUCCGCUGCCAAGUUCGGAAUGCACCUU
CAAGCAGCGACUCCCAAGGGAUACGAACCUGAUGCCUCCGUGACUAAGCUGG
CAGAGCAGUACGCCAAGGAGAACGGUACAAAGCUGCUGCUCACGAACGACCC
CCUGGAGGCGGCCCACGGCGGAAACGUGCUGAUUACCGAUACCUGGAUCUCA
AUGGGCCAGGAAGAGGAGAAGAAGAAGCGGCUCCAGGCGUUUCAAGGCUACC
AGGUCACCAUGAAAACCGCGAAGGUCGCCGCCUCCGACUGGACUUUCUUGCA
CUGCCUGCCGCGGAAGCCCGAGGAAGUGGAUGACGAAGUGUUCUACUCGCCG
AGAUCGUUGGUGUUCCCUGAGGCCGAAAACAGGAAGUGGACCAUCAUGGCCG
UGAUGGUGUCCCUGCUGACUGAUUACAGCCCACAGCUGCAGAAGCCUAAGUU
CUAGCUCGAGCUAGUGACUGACUAGGAUCUGGUUACCACUAAACCAGCCUCA
AGAACACCCGAAUGGAGUCUCUAAGCUACAUAAUACCAACUUACACUUACAA
AAUGUUGUCCCCCAAAAUGUAGCCAUUCGUAUCUGCUCCUAAUAAAAAGAAA
GUUUCUUCACAUUCUAG
>mARM571
(SEQ ID NO: 34)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAAUCUCCGCAUC
CUCCUUAACAACGCCGCGUUUAGAAACGGCCACAACUUCAUGGUCCGGAACU
UCAGAUGUGGCCAGCCGCUUCAAAACAAGGUCCAGCUGAAGGGCCGGGAUCU
UCUGACCCUGAAGAACUUUACUGGCGAAGAGAUCAAGUACAUGCUCUGGCUC
UCCGCGGACUUGAAGUUCCGCAUUAAGCAGAAGGGGGAAUACCUUCCGCUGC
UUCAAGGAAAGAGCCUCGGCAUGAUCUUUGAGAAGCGCUCAACCAGGACCCG
CCUUUCUACUGAAACUGGGUUCGCGCUGCUCGGUGGCCACCCCUGCUUCCUG
ACGACCCAGGACAUCCACCUCGGAGUGAACGAAUCCCUCACCGAUACCGCCC
GGGUGUUAUCGAGCAUGGCAGAUGCCGUGCUGGCCAGGGUGUACAAACAGUC
CGAUCUGGACACUCUGGCCAAGGAGGCGUCAAUUCCUAUUAUCAACGGCCUU
AGUGACCUCUACCAUCCGAUUCAGAUCCUGGCCGAUUACCUCACCCUGCAAG
AACACUACAGCUCCCUGAAGGGUCUGACAUUGUCCUGGAUCGGCGACGGCAA
CAACAUUCUCCAUUCCAUCAUGAUGUCCGCCGCAAAAUUCGGCAUGCAUCUU
CAAGCCGCCACGCCGAAGGGUUACGAGCCCGACGCUUCCGUGACUAAGCUCG
CCGAGCAGUACGCUAAGGAGAACGGAACCAAGCUUCUGCUGACUAACGACCC
ACUAGAAGCAGCCCACGGGGGCAACGUGCUUAUUACUGACACCUGGAUCUCC
AUGGGCCAGGAAGAAGAGAAAAAGAAGCGGCUGCAGGCGUUCCAGGGAUAU
CAGGUCACCAUGAAAACCGCCAAGGUCGCUGCCUCCGACUGGACCUUCCUGC
ACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCC
GUGAUGGUGUCACUGCUCACCGACUACAGCCCGCAGCUUCAGAAGCCCAAGU
UCUAGCUCGAGCUAGUGACUGACUAGGAUCUGGUUACCACUAAACCAGCCUC
AAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAA
AGUUUCUUCACAUUCUAG
>mARM572
(SEQ ID NO: 35)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAACCUGAGAAUC
CUCCUGAACAACGCCGCCUUCCGCAAUGGUCAUAACUUCAUGGUCCGCAACU
UUCGCUGCGGACAGCCUCUCCAAAACAAGGUCCAGCUCAAGGGGCGCGACCU
CCUCACACUGAAGAACUUCACUGGAGAAGAAAUCAAGUACAUGCUGUGGCUG
AGCGCCGAUCUGAAGUUCCGGAUCAAGCAGAAGGGAGAGUACCUUCCUCUGC
UGCAAGGGAAGUCCUUGGGAAUGAUUUUCGAGAAGCGGUCCACCCGGACCAG
GCUGAGCACUGAAACUGGCUUCGCCCUGCUGGGAGGCCACCCUUGUUUCCUG
ACCACUCAGGACAUCCACCUGGGCGUGAACGAGUCCCUGACCGAUACUGCCA
GAGUGCUGUCCUCCAUGGCCGACGCCGUGCUCGCCCGGGUGUACAAGCAGUC
AGACCUCGAUACGCUGGCCAAGGAAGCCUCCAUUCCCAUUAUCAAUGGUCUG
UCGGACCUCUACCAUCCAAUCCAAAUCCUCGCCGACUACCUGACUCUGCAAG
AACACUACAGCUCACUCAAGGGCCUCACCCUCUCCUGGAUCGGCGACGGAAA
CAACAUCCUUCACUCGAUUAUGAUGUCGGCCGCGAAGUUCGGGAUGCACCUC
CAAGCUGCCACUCCAAAAGGCUACGAGCCGGAUGCCUCAGUGACUAAGUUGG
CGGAACAGUAUGCGAAGGAGAACGGUACCAAGCUCCUGCUGACUAACGACCC
GCUGGAGGCCGCCCACGGGGGAAACGUGCUCAUCACCGAUACUUGGAUUUCC
AUGGGACAGGAGGAAGAGAAGAAGAAGCGGUUGCAGGCAUUUCAGGGCUAC
CAGGUCACCAUGAAAACUGCCAAAGUCGCCGCCAGCGACUGGACCUUCCUGC
ACUGCCUGCCCCGCAAGCCUGAAGAAGUGGACGACGAGGUGUUCUACUCUCC
CCGGUCCCUCGUGUUCCCUGAGGCCGAAAACAGGAAGUGGACCAUCAUGGCU
GUGAUGGUGUCCCUCCUGACCGACUACAGCCCUCAGCUCCAAAAACCCAAGU
UUUAGCUCGAGCUAGUGACUGACUAGGAUCUGGUUACCACUAAACCAGCCUC
AAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAA
AGUUUCUUCACAUUCUAG
>mARM573
(SEQ ID NO: 36)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAACCUGAGAAUC
CUCUUGAACAAUGCUGCUUUUCGGAAUGGCCACAACUUUAUGGUUCGGAACU
UCCGUUGCGGCCAGCCUUUACAAAACAAGGUCCAGCUGAAGGGCCGGGAUUU
GCUCACACUGAAGAACUUUACUGGGGAGGAGAUUAAGUAUAUGCUGUGGCU
GUCCGCUGACCUGAAGUUUAGGAUCAAGCAGAAGGGCGAAUAUCUGCCGCUG
CUGCAAGGGAAAAGUCUGGGCAUGAUUUUUGAAAAGCGCUCUACCCGGACCA
GACUGUCUACGGAAACAGGCUUUGCCCUGCUGGGCGGCCACCCCUGUUUUCU
GACAACGCAGGACAUCCAUCUGGGCGUGAACGAAUCACUGACCGAUACUGCU
CGGGUACUCAGUUCUAUGGCUGACGCAGUGCUGGCUAGGGUGUACAAGCAGA
GCGACUUGGACACACUGGCUAAGGAGGCCAGCAUCCCCAUUAUCAAUGGCCU
GUCUGAUUUGUACCAUCCCAUUCAAAUCCUGGCUGAUUAUCUGACACUACAA
GAGCAUUACUCAAGUCUGAAGGGUUUGACUCUCUCCUGGAUCGGCGACGGCA
ACAACAUUUUACAUUCCAUUAUGAUGAGUGCUGCUAAGUUUGGCAUGCAUU
UGCAAGCUGCUACCCCAAAGGGCUAUGAACCUGACGCUAGCGUAACCAAGUU
GGCCGAACAGUAUGCUAAAGAGAAUGGCACCAAGCUGCUCCUGACGAAUGAC
CCCCUGGAAGCUGCUCAUGGCGGAAACGUACUUAUAACUGAUACAUGGAUUA
GCAUGGGCCAGGAAGAGGAGAAGAAGAAGAGACUGCAGGCCUUCCAAGGCUA
UCAGGUCACCAUGAAAACUGCCAAGGUUGCAGCUAGCGACUGGACCUUCCUG
CACUGUUUGCCGAGGAAACCCGAGGAGGUGGACGAUGAAGUCUUUUAUUCUC
CCCGCUCCUUGGUGUUUCCCGAGGCUGAAAAUCGAAAGUGGACGAUAAUGGC
AGUGAUGGUGUCCCUACUGACCGACUAUUCUCCACAACUGCAGAAGCCUAAA
UUCUAGCUCGAGCUAGUGACUGACUAGGAUCUGGUUACCACUAAACCAGCCU
CAAGAACACCCGAAUGGAGUCUCUAAGCUACAUAAUACCAACUUACACUUAC
AAAAUGUUGUCCCCCAAAAUGUAGCCAUUCGUAUCUGCUCCUAAUAAAAAGA
AAGUUUCUUCACAUUCUAG
>mARM574
(SEQ ID NO: 37)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAAUCUGAGGAUC
CUGCUGAACAACGCUGCUUUUCGCAACGGUCAUAACUUUAUGGUUCGCAAUU
UUCGUUGUGGCCAGCCGCUGCAGAACAAGGUUCAGCUGAAGGGCAGAGAUCU
GCUGACUCUGAAGAACUUCACUGGGGAAGAAAUCAAGUAUAUGUUAUGGCU
GUCCGCGGAUCUGAAAUUUCGAAUCAAGCAGAAGGGCGAAUAUCUUCCCCUG
CUGCAAGGGAAAUCCUUGGGCAUGAUUUUUGAGAAGAGGAGCACUAGGACU
AGAUUGUCAACAGAAACAGGCUUUGCUUUGUUGGGCGGACAUCCCUGCUUUC
UGACGACACAGGAUAUCCACCUCGGCGUAAACGAGUCCCUCACCGACACUGC
UAGGGUACUGAGCAGCAUGGCCGACGCUGUGCUAGCCCGGGUUUACAAGCAG
UCAGACCUGGACACCCUUGCCAAGGAAGCUUCUAUUCCAAUUAUCAACGGCC
UGAGUGACCUGUAUCACCCUAUUCAAAUACUCGCCGACUAUUUGACGCUUCA
AGAACAUUACAGCAGCCUCAAGGGCUUAACCUUGAGUUGGAUAGGCGACGGC
AACAAUAUCCUGCAUUCCAUUAUGAUGUCUGCCGCUAAGUUUGGCAUGCAUC
UACAAGCCGCAACACCCAAGGGCUAUGAACCCGACGCUAGCGUGACCAAGCU
GGCCGAGCAGUAUGCUAAGGAAAAUGGCACAAAGCUCCUUCUUACCAACGAU
CCCCUGGAGGCUGCUCACGGCGGCAACGUGCUGAUUACCGAUACAUGGAUUA
GCAUGGGCCAGGAGGAGGAGAAAAAGAAGCGGCUCCAGGCUUUUCAAGGCUA
UCAGGUCACCAUGAAAACUGCAAAGGUCGCUGCCUCCGACUGGACUUUCCUG
CAUUGUCUACCCCGCAAGCCUGAGGAAGUGGACGAUGAGGUGUUCUACUCCC
CACGGAGUCUGGUGUUCCCGGAAGCAGAGAAUCGGAAGUGGACCAUCAUGGC
UGUCAUGGUGUCGCUCUUGACUGACUAUUCUCCCCAACUGCAAAAACCCAAG
UUUUAGCUCGAGCUAGUGACUGACUAGGAUCUGGUUACCACUAAACCAGCCU
CAAGAACACCCGAAUGGAGUCUCUAAGCUACAUAAUACCAACUUACACUUAC
AAAAUGUUGUCCCCCAAAAUGUAGCCAUUCGUAUCUGCUCCUAAUAAAAAGA
AAGUUUCUUCACAUUCUAG
>mARM575
(SEQ ID NO: 38)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGUUAUUCAACCUUCGUAUC
CUGCUAAACAAUGCUGCUUUUCGCAAUGGCCAUAACUUUAUGGUUCGCAACU
UUAGAUGCGGCCAGCCGCUGCAGAACAAGGUUCAGCUGAAGGGCCGGGACUU
GCUGACGCUGAAAAACUUUACCGGGGAAGAGAUUAAGUAUAUGCUGUGGCU
AAGCGCUGAUCUGAAGUUUAGGAUCAAGCAGAAGGGCGAAUAUCUGCCACUG
CUGCAAGGGAAGAGUCUUGGCAUGAUUUUUGAAAAGCGGUCUACCAGAACCC
GGCUGUCGACCGAGACAGGUUUUGCUCUGCUGGGGGGCCAUCCCUGUUUUCU
GACAACUCAGGACAUUCACCUGGGCGUGAAUGAGUCCCUGACCGAUACUGCU
AGGGUGUUGAGUAGCAUGGCCGACGCUGUACUCGCUCGAGUGUAUAAGCAGU
CUGAUCUGGACACUCUGGCUAAGGAAGCUUCCAUUCCUAUUAUCAACGGCUU
GAGCGACCUGUACCACCCCAUUCAAAUCCUCGCUGAUUACUUGACUUUGCAA
GAACAUUACAGCAGCUUGAAGGGCUUAACACUGAGCUGGAUAGGCGACGGAA
ACAACAUCUUGCAUUCCAUAAUGAUGUCCGCCGCUAAGUUCGGGAUGCACCU
CCAAGCAGCCACACCCAAGGGCUAUGAACCGGAUGCUUCCGUGACAAAACUG
GCUGAGCAGUAUGCUAAGGAGAAUGGCACGAAACUGCUGCUCACCAACGACC
CAUUGGAAGCUGCACAUGGUGGCAACGUACUGAUCACUGACACUUGGAUCUC
AAUGGGCCAGGAGGAAGAGAAGAAGAAAAGGCUGCAGGCAUUUCAGGGAUA
CCAAGUCACUAUGAAAACUGCCAAGGUCGCUGCCUCCGACUGGACAUUCCUG
CAUUGUCUGCCACGGAAGCCUGAGGAAGUCGAUGACGAAGUGUUCUAUAGCC
CACGAAGCUUGGUGUUUCCCGAGGCUGAGAAUAGGAAGUGGACCAUUAUGGC
UGUUAUGGUGUCCCUGCUCACCGACUAUUCCCCUCAACUGCAAAAACCCAAG
UUUUAGCUCGAGCUAGUGACUGACUAGGAUCUGGUUACCACUAAACCAGCCU
CAAGAACACCCGAAUGGAGUCUCUAAGCUACAUAAUACCAACUUACACUUAC
AAAAUGUUGUCCCCCAAAAUGUAGCCAUUCGUAUCUGCUCCUAAUAAAAAGA
AAGUUUCUUCACAUUCUAG
>mARM708
(SEQ ID NO: 39)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCAUGCUUUUUAAUCUCCGCAUCCUC
CUUAACAACGCCGCGUUUAGAAACGGCCACAACUUCAUGGUCCGGAACUUCA
GAUGUGGCCAGCCGCUUCAAAACAAGGUCCAGCUGAAGGGCCGGGAUCUUCU
GACCCUGAAGAACUUUACUGGCGAAGAGAUCAAGUACAUGCUCUGGCUCUCC
GCGGACUUGAAGUUCCGCAUUAAGCAGAAGGGGGAAUACCUUCCGCUGCUUC
AAGGAAAGAGCCUCGGCAUGAUCUUUGAGAAGCGCUCAACCAGGACCCGCCU
UUCUACUGAAACUGGGUUCGCGCUGCUCGGUGGCCACCCCUGCUUCCUGACG
ACCCAGGACAUCCACCUCGGAGUGAACGAAUCCCUCACCGAUACCGCCCGGG
UGUUAUCGAGCAUGGCAGAUGCCGUGCUGGCCAGGGUGUACAAACAGUCCGA
UCUGGACACUCUGGCCAAGGAGGCGUCAAUUCCCAUCAUCAACGGCCUGAGC
GACCUGUACCACCCAAUCCAAAUCCUGGCUGACUACCUGACCCUGCAAGAGC
ACUACAGCAGCCUGAAGGGUCUGACCCUGUCAUGGAUUGGCGAUGGAAACAA
UAUUCUGCACUCCAUCAUGAUGUCCGCCGCGAAGUUCGGAAUGCAUCUGCAA
GCCGCCACGCCAAAAGGAUACGAACCGGAUGCGUCCGUGACGAAGUUGGCGG
AACAGUACGCGAAGGAGAACGGAACCAAGCUUCUGCUGACUAACGACCCCCU
CGAGGCUGCGCAUGGGGGCAACGUGCUGAUUACCGACACCUGGAUCUCCAUG
GGGCAGGAGGAAGAGAAGAAGAAGAGACUGCAGGCAUUCCAGGGGUACCAG
GUCACCAUGAAAACCGCAAAAGUGGCAGCUUCGGACUGGACUUUCCUGCAUU
GCCUGCCGAGGAAGCCGGAGGAAGUCGACGACGAAGUGUUCUACUCGCCUCG
GUCCCUGGUGUUCCCCGAGGCCGAAAACCGGAAGUGGACCAUCAUGGCCGUG
AUGGUGUCCUUGCUGACUGACUAUAGCCCGCAGCUGCAGAAGCCUAAGUUCU
AGCUCGAGCUAGUGACUGACUAGGAUCUGGUUACCACUAAACCAGCCUCAAG
AACACCCGAAUGGAGUCUCUAAGCUACAUAAUACCAACUUACACUUACAAAA
UGUUGUCCCCCAAAAUGUAGCCAUUCGUAUCUGCUCCUAAUAAAAAGAAAGU
UUCUUCACAUUCUAG
>mARM709
(SEQ ID NO: 40)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCAUGCUUUUCAACCUGAGAAUCCUC
UUGAACAAUGCUGCUUUUCGGAAUGGCCACAACUUUAUGGUUCGGAACUUCC
GUUGCGGCCAGCCUUUACAAAACAAGGUCCAGCUGAAGGGCCGGGAUUUGCU
CACACUGAAGAACUUUACUGGAGAAGAGAUCAAGUACAUGCUGUGGCUGUCG
GCCGACCUGAAGUUCAGGAUCAAGCAGAAGGGAGAAUACCUUCCGCUGCUUC
AAGGAAAGAGCCUCGGCAUGAUCUUUGAGAAGCGCUCAACCAGGACCCGCCU
UUCUACUGAAACUGGGUUCGCGCUGCUCGGUGGCCACCCCUGCUUCCUGACG
ACCCAGGACAUCCACCUCGGAGUGAACGAAUCCCUCACCGAUACCGCCCGGG
UGUUAUCGAGCAUGGCAGAUGCCGUGCUGGCCAGGGUGUACAAACAGUCCGA
UCUCGAUACCUUGGCAAAGGAGGCUUCCAUUCCCAUCAUCAACGGCCUGAGC
GACCUGUACCACCCAAUCCAAAUCCUGGCUGACUACCUGACCCUGCAAGAGC
ACUACAGCAGCCUGAAGGGUCUGACCCUGUCAUGGAUUGGCGAUGGAAACAA
UAUUCUGCACUCCAUCAUGAUGUCCGCCGCGAAGUUCGGAAUGCAUCUGCAA
GCCGCCACUCCAAAAGGAUACGAACCGGAUGCGUCCGUGACCAAGUUGGCGG
AACAGUACGCGAAGGAGAACGGAACCAAGCUUCUGCUGACUAACGACCCCCU
CGAGGCUGCGCAUGGGGGCAACGUGCUGAUUACCGACACCUGGAUCUCCAUG
GGGCAGGAGGAAGAGAAGAAGAAGAGACUGCAGGCAUUCCAGGGGUACCAG
GUCACCAUGAAAACCGCAAAAGUGGCAGCUUCGGACUGGACUUUCCUGCAUU
GCCUGCCGAGGAAGCCGGAGGAAGUCGACGACGAAGUGUUCUACUCGCCUCG
GUCCCUGGUGUUCCCCGAGGCCGAAAACCGGAAGUGGACCAUCAUGGCCGUG
AUGGUGUCCUUGCUGACUGACUAUAGCCCGCAGCUGCAGAAGCCUAAGUUCU
AGCUCGAGCUAGUGACUGACUAGGAUCUGGUUACCACUAAACCAGCCUCAAG
AACACCCGAAUGGAGUCUCUAAGCUACAUAAUACCAACUUACACUUACAAAA
UGUUGUCCCCCAAAAUGUAGCCAUUCGUAUCUGCUCCUAAUAAAAAGAAAGU
UUCUUCACAUUCUAG
>mARM710
(SEQ ID NO: 41)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCAUGCUUUUCAACCUGAGAAUCCUC
UUGAACAAUGCUGCUUUUCGGAAUGGCCACAACUUUAUGGUUCGGAACUUCC
GUUGCGGCCAGCCUUUACAAAACAAGGUCCAGCUGAAGGGCCGGGAUUUGCU
CACACUAAAGAACUUUACUGGAGAAGAGAUCAAGUACAUGCUAUGGCUGUCG
GCCGACCUGAAGUUCCGUAUCAAGCAGAAGGGAGAAUACCUUCCGCUGCUUC
AAGGAAAGAGCCUCGGCAUGAUCUUUGAGAAGCGCUCAACCAGGACCCGCCU
UUCUACUGAAACUGGGUUCGCGCUGCUCGGUGGCCACCCCUGCUUCCUGACG
ACCCAGGACAUCCACCUCGGAGUGAACGAAUCCCUCACCGAUACCGCCCGGG
UGUUAUCGAGCAUGGCAGAUGCCGUGCUGGCCAGGGUGUACAAACAGUCCGA
UCUCGAUACCUUGGCAAAGGAGGCUUCCAUUCCCAUCAUCAACGGCCUGAGC
GACCUGUACCACCCAAUCCAAAUCCUGGCUGACUACCUGACCCUGCAAGAGC
ACUACAGCAGCCUGAAGGGUCUGACCCUGUCAUGGAUUGGCGAUGGAAACAA
UAUUCUGCACUCCAUCAUGAUGUCCGCCGCGAAGUUCGGAAUGCAUCUGCAA
GCCGCCACUCCAAAAGGAUACGAACCGGAUGCAUCCGUGACCAAGUUGGCGG
AACAGUACGCGAAGGAGAACGGAACCAAGCUCCUGCUGACUAACGACCCGCU
CGAGGCUGCGCAUGGGGGUAACGUGCUGAUUACGGACACCUGGAUCUCCAUG
GGGCAGGAGGAAGAGAAGAAGAAGAGACUGCAGGCAUUCCAGGGGUACCAG
GUCACCAUGAAAACCGCAAAAGUGGCAGCUUCGGACUGGACUUUCCUGCAUU
GCCUGCCGAGGAAGCCGGAGGAAGUCGACGACGAAGUGUUCUACUCGCCUCG
GUCCCUGGUGUUCCCCGAGGCCGAAAACCGGAAGUGGACCAUCAUGGCCGUG
AUGGUGUCCUUGCUGACUGACUAUAGCCCGCAGCUGCAGAAGCCUAAGUUCU
AGCUCGAGCUAGUGACUGACUAGGAUCUGGUUACCACUAAACCAGCCUCAAG
AACACCCGAAUGGAGUCUCUAAGCUACAUAAUACCAACUUACACUUACAAAA
UGUUGUCCCCCAAAAUGUAGCCAUUCGUAUCUGCUCCUAAUAAAAAGAAAGU
UUCUUCACAUUCUAG
>mARM711
(SEQ ID NO: 42)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCAUGCUUUUCAACCUGAGAAUCCUC
UUGAACAAUGCUGCUUUUCGGAAUGGCCACAACUUUAUGGUUCGGAACUUCC
GUUGCGGCCAGCCUUUACAAAACAAGGUCCAGCUGAAGGGCCGGGAUUUGCU
CACACUAAAGAACUUUACUGGAGAAGAGAUCAAGUACAUGCUAUGGCUGUCG
GCCGACCUGAAGUUCCGUAUCAAGCAGAAGGGAGAAUACCUUCCGCUGCUUC
AAGGAAAGAGCCUCGGCAUGAUCUUUGAGAAGCGCUCAACCAGGACCCGCCU
UUCUACUGAAACUGGGUUCGCGCUGCUCGGUGGCCACCCCUGCUUCCUGACG
ACCCAGGACAUCCACCUCGGAGUGAACGAAUCCCUCACCGAUACCGCCCGGG
UGUUAUCGAGCAUGGCAGAUGCCGUGCUGGCCAGGGUGUACAAACAGUCCGA
UCUCGAUACCUUGGCAAAGGAGGCUUCCAUUCCCAUCAUCAACGGCCUGAGC
GACCUGUACCACCCAAUCCAAAUCCUGGCUGACUACCUGACCCUGCAAGAGC
ACUACAGCAGCCUGAAGGGUCUGACCCUGUCAUGGAUUGGCGAUGGAAACAA
UAUUCUGCACUCCAUCAUGAUGUCCGCCGCGAAGUUCGGAAUGCAUCUGCAA
GCCGCCACUCCAAAAGGAUACGAACCGGAUGCGUCCGUGACCAAGUUGGCGG
AACAGUACGCGAAGGAGAACGGAACCAAGCUUCUGCUGACUAACGACCCCCU
CGAGGCUGCGCAUGGGGGCAACGUGCUGAUUACCGACACCUGGAUCUCCAUG
GGGCAGGAGGAAGAGAAGAAGAAGAGACUGCAGGCAUUCCAGGGGUACCAG
GUCACCAUGAAAACCGCAAAAGUGGCAGCUUCGGACUGGACUUUCCUGCAUU
GCCUGCCGAGGAAGCCGGAGGAAGUCGACGACGAAGUGUUCUACUCGCCUCG
GUCCCUGGUGUUCCCCGAGGCCGAAAACCGGAAGUGGACCAUCAUGGCCGUG
AUGGUGUCCUUGCUGACUGACUAUAGCCCGCAGCUGCAGAAGCCUAAGUUCU
AGCUCGAGCUAGUGACUGACUAGGAUCUGGUUACCACUAAACCAGCCUCAAG
AACACCCGAAUGGAGUCUCUAAGCUACAUAAUACCAACUUACACUUACAAAA
UGUUGUCCCCCAAAAUGUAGCCAUUCGUAUCUGCUCCUAAUAAAAAGAAAGU
UUCUUCACAUUCUAG
>mARM712
(SEQ ID NO: 43)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUGUUCAACCUGCGAAUC
CUGCUGAACAACGCCGCUUUUCGGAACGGGCACAACUUUAUGGUGAGGAACU
UUCGCUGCGGACAGCCCCUCCAGAAUAAGGUCCAGCUGAAGGGCAGGGACCU
GCUGACCCUGAAAAAUUUCACAGGGGAGGAAAUCAAGUAUAUGCUGUGGCU
GUCAGCUGAUCUGAAGUUCCGGAUCAAGCAGAAGGGCGAAUAUCUGCCUCUG
CUCCAGGGCAAAAGCCUGGGGAUGAUCUUCGAAAAGCGCAGUACUCGGACCA
GACUGUCAACCGAGACUGGAUUCGCUCUGCUGGGAGGACACCCUUGUUUUCU
GACCACUCAGGACAUUCACCUGGGAGUGAACGAGUCCCUGACCGACACUGCU
CGCGUCCUGAGCUCUAUGGCCGACGCUGUGCUGGCUCGAGUCUACAAACAGU
CCGACCUGGAUACCCUGGCCAAGGAAGCUUCUAUCCCAAUUAUUAACGGCCU
GUCAGACCUGUAUCACCCCAUCCAGAUUCUGGCCGAUUACCUGACCCUCCAG
GAGCACUAUUCUAGUCUGAAAGGGCUGACACUGAGUUGGAUUGGGGACGGA
AACAAUAUCCUGCACUCUAUUAUGAUGUCAGCCGCCAAGUUUGGAAUGCACC
UCCAGGCUGCAACCCCAAAAGGCUACGAACCCGAUGCCUCAGUGACAAAGCU
GGCUGAACAGUACGCCAAAGAGAACGGCACUAAGCUGCUGCUGACCAACGAC
CCUCUGGAGGCCGCUCACGGAGGCAACGUGCUGAUCACCGAUACCUGGAUUA
GUAUGGGACAGGAGGAAGAGAAGAAGAAGCGGCUCCAGGCCUUCCAGGGCUA
CCAGGUGACAAUGAAAACCGCUAAGGUCGCAGCCAGCGAUUGGACCUUUCUG
CACUGCCUGCCCAGAAAGCCCGAAGAGGUGGACGACGAGGUCUUCUACUCUC
CCAGAAGCCUGGUGUUUCCCGAAGCUGAGAAUAGGAAGUGGACAAUUAUGGC
AGUGAUGGUCAGCCUGCUGACUGAUUAUUCACCUCAGCUCCAGAAACCAAAG
UUCUGAUAACUCGAGCUAGUGACUGACUAGGAUCUGGUUACCACUAAACCAG
CCUCAAGAACACCCGAAUGGAGUCUCUAAGCUACAUAAUACCAACUUACACU
UACAAAAUGUUGUCCCCCAAAAUGUAGCCAUUCGUAUCUGCUCCUAAUAAAA
AGAAAGUUUCUUCACAUUCUAG
>mARM713
(SEQ ID NO: 44)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUGUUCAACCUGCGCAUC
CUGCUGAACAACGCCGCCUUCCGCAACGGCCACAACUUCAUGGUGCGCAACU
UCCGCUGCGGCCAGCCCCUGCAGAACAAGGUGCAGCUGAAGGGCCGCGACCU
GCUGACCCUGAAGAACUUCACCGGCGAGGAGAUCAAGUACAUGCUGUGGCUG
AGCGCCGACCUGAAGUUCCGCAUCAAGCAGAAGGGCGAGUACCUGCCCCUGC
UGCAGGGCAAGAGCCUGGGCAUGAUCUUCGAGAAGCGCAGCACCCGCACCCG
CCUGAGCACCGAGACAGGCCUGGCCCUGCUGGGCGGCCACCCCUGCUUCCUG
ACCACCCAGGACAUCCACCUGGGCGUGAACGAGAGCCUGACCGACACCGCCC
GCGUGCUGAGCAGCAUGGCCGACGCCGUGCUGGCCCGCGUGUACAAGCAGAG
CGACCUGGACACCCUGGCCAAGGAGGCCAGCAUCCCCAUCAUCAACGGCCUG
AGCGACCUGUACCACCCCAUCCAGAUCCUGGCCGACUACCUGACCCUGCAGG
AGCACUACAGCAGCCUGAAGGGCCUGACCCUGAGCUGGAUCGGCGACGGCAA
CAACAUCCUGCACAGCAUCAUGAUGAGCGCCGCCAAGUUCGGCAUGCACCUG
CAGGCCGCCACCCCCAAGGGCUACGAGCCCGACGCCAGCGUGACCAAGCUGGC
CGAGCAGUACGCCAAGGAGAACGGCACCAAGCUGCUGCUGACCAACGACCCC
CUGGAGGCCGCCCACGGCGGCAACGUGCUGAUCACCGACACCUGGAUCAGCA
UGGGCCAGGAGGAGGAGAAGAAGAAGCGCCUGCAGGCCUUCCAGGGCUACCA
GGUGACCAUGAAGACCGCCAAGGUGGCCGCCAGCGACUGGACCUUCCUGCAC
UGCCUGCCCCGCAAGCCCGAGGAGGUGGACGACGAGGUGUUCUACAGCCCCC
GCAGCCUGGUGUUCCCCGAGGCCGAGAACCGCAAGUGGACCAUCAUGGCCGU
GAUGGUGAGCCUGCUGACCGACUACAGCCCCCAGCUGCAGAAGCCCAAGUUC
UGAUAACUCGAGCUAGUGACUGACUAGGAUCUGGUUACCACUAAACCAGCCU
CAAGAACACCCGAAUGGAGUCUCUAAGCUACAUAAUACCAACUUACACUUAC
AAAAUGUUGUCCCCCAAAAUGUAGCCAUUCGUAUCUGCUCCUAAUAAAAAGA
AAGUUUCUUCACAUUCUAG
>mARM714
(SEQ ID NO: 45)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUGUUCAACCUGCGAAUC
CUGCUGAACAACGCCGCUUUUCGGAACGGGCACAACUUUAUGGUGAGGAACU
UUCGCUGCGGACAGCCCCUCCAGAAUAAGGUCCAGCUGAAGGGCAGGGACCU
GCUGACCCUGAAAAAUUUCACAGGGGAGGAAAUCAAGUAUAUGCUGUGGCU
GUCAGCUGAUCUGAAGUUCCGGAUCAAGCAGAAGGGCGAAUAUCUGCCUCUG
CUCCAGGGCAAAAGCCUGGGGAUGAUCUUCGAAAAGCGCAGUACUCGGACCA
GACUGUCAACCGAGACUGGAUUCGCUCUGCUGGGAGGACACCCUUGUUUUCU
GACCACUCAGGACAUUCACCUGGGAGUGAACGAGUCCCUGACCGACACUGCU
CGCGUCCUGAGCUCUAUGGCCGACGCUGUGCUAGCUCGAGUCUACAAACAGU
CCGACCUGGAUACCCUGGCCAAGGAAGCUUCUAUCCCAAUUAUUAACGGCCU
GUCAGACCUGUAUCACCCCAUCCAGAUUCUGGCCGAUUACCUGACCCUCCAG
GAGCACUAUUCUAGUCUGAAAGGGCUGACACUGAGUUGGAUUGGGGACGGA
AACAAUAUCCUGCACUCUAUUAUGAUGUCAGCCGCCAAGUUUGGAAUGCACC
UCCAGGCUGCAACCCCAAAAGGCUACGAACCCGAUGCCUCAGUGACAAAGCU
GGCUGAACAGUACGCCAAAGAGAACGGCACUAAGCUGCUGCUGACCAACGAC
CCUCUGGAGGCCGCUCACGGAGGCAACGUGCUGAUCACCGAUACCUGGAUUA
GUAUGGGACAGGAGGAAGAGAAGAAGAAGCGGCUCCAGGCCUUCCAGGGCUA
CCAGGUGACAAUGAAAACCGCUAAGGUCGCAGCCAGCGAUUGGACCUUUCUG
CACUGCCUGCCCAGAAAGCCCGAAGAGGUGGACGACGAGGUCUUCUACUCUC
CCAGAAGCCUGGUGUUUCCCGAAGCUGAGAAUAGGAAGUGGACAAUUAUGGC
AGUGAUGGUCAGCCUGCUGACUGAUUAUUCACCUCAGCUCCAGAAACCAAAG
UUCUGAUAACUCGAGCUAGUGACUGACUAGGAUCUGGUUACCACUAAACCAG
CCUCAAGAACACCCGAAUGGAGUCUCUAAGCUACAUAAUACCAACUUACACU
UACAAAAUGUUGUCCCCCAAAAUGUAGCCAUUCGUAUCUGCUCCUAAUAAAA
AGAAAGUUUCUUCACAUUCUAG
>mARM715
(SEQ ID NO: 46)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUGUUCAACCUGCGAAUC
CUGCUGAACAAUGCCGCUUUUCGGAACGGGCACAAUUUCAUGGUGAGGAACU
UUCGCUGCGGACAGCCCCUCCAGAACAAGGUCCAGCUGAAGGGCAGGGACCU
GCUGACCCUGAAAAAUUUCACAGGGGAGGAAAUCAAGUACAUGCUGUGGCUG
UCAGCCGAUCUGAAGUUCCGGAUCAAGCAGAAGGGCGAAUAUCUGCCUCUGC
UCCAGGGCAAAAGCCUGGGGAUGAUCUUCGAAAAGCGCAGUACUCGGACCAG
ACUGUCAACAGAGACUGGAUUCGCACUGCUGGGAGGACACCCAUGUUUUCUG
ACCACACAGGACAUUCAUCUGGGAGUGAACGAGUCCCUGACCGACACAGCAC
GCGUCCUGAGCUCCAUGGCUGAUGCAGUGCUGGCUCGAGUCUACAAACAGUC
UGACCUGGAUACCCUGGCCAAGGAAGCUUCUAUCCCAAUCAUUAAUGGCCUG
AGUGACCUGUAUCACCCCAUCCAGAUUCUGGCCGAUUACCUGACCCUCCAGG
AGCAUUAUUCUAGUCUGAAAGGGCUGACACUGAGCUGGAUUGGGGACGGAA
ACAAUAUCCUGCACUCCAUUAUGAUGAGCGCCGCCAAGUUUGGAAUGCACCU
CCAGGCUGCAACCCCAAAAGGCUACGAACCCGAUGCCUCCGUGACAAAGCUG
GCAGAACAGUAUGCCAAAGAGAACGGCACUAAGCUGCUGCUGACCAAUGACC
CUCUGGAGGCCGCUCACGGAGGCAACGUGCUGAUCACUGAUACCUGGAUUAG
UAUGGGACAGGAGGAAGAGAAGAAGAAGCGGCUCCAGGCCUUCCAGGGCUAC
CAGGUGACAAUGAAAACUGCUAAGGUCGCAGCCAGCGACUGGACCUUUCUGC
AUUGCCUGCCCAGAAAGCCUGAAGAGGUGGACGAUGAGGUCUUCUACUCACC
CAGAAGCCUGGUGUUUCCUGAAGCUGAGAAUAGGAAGUGGACAAUCAUGGC
AGUGAUGGUCAGCCUGCUGACUGAUUAUUCCCCUCAGCUCCAGAAACCAAAG
UUCUGAUAACUCGAGCUAGUGACUGACUAGGAUCUGGUUACCACUAAACCAG
CCUCAAGAACACCCGAAUGGAGUCUCUAAGCUACAUAAUACCAACUUACACU
UACAAAAUGUUGUCCCCCAAAAUGUAGCCAUUCGUAUCUGCUCCUAAUAAAA
AGAAAGUUUCUUCACAUUCUAG
>mARM716
(SEQ ID NO: 47)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAACCUUCGCAUU
CUCCUCAACAACGCCGCGUUUAGAAACGGACACAACUUCAUGGUCCGCAACU
UCCGCUGCGGACAGCCGCUGCAGAACAAGGUCCAGCUCAAGGGUCGGGAUCU
CCUGACGCUGAAGAACUUUACCGGCGAAGAGAUUAAGUACAUGCUGUGGCUG
UCCGCCGACCUUAAGUUCCGGAUCAAGCAGAAGGGCGAAUACCUUCCCCUGC
UGCAAGGAAAGUCCCUGGGCAUGAUCUUCGAGAAGCGCAGUACCAGAACCAG
ACUCUCCACUGAAACCGGGUUCGCGCUGCUUGGCGGCCACCCGUGUUUCCUC
ACUACGCAAGACAUCCAUCUUGGCGUGAACGAGUCCCUUACCGACACCGCCA
GGGUGCUGUCAAGCAUGGCCGACGCCGUCCUUGCGCGCGUGUACAAGCAGUC
AGACCUUGAUACUCUGGCCAAGGAAGCCUCCAUCCCUAUUAUCAACGGCCUA
UCCGACCUUUACCACCCGAUCCAGAUCCUCGCUGACUACCUGACCCUGCAAG
AACACUACAGCAGCCUCAAGGGACUGACUCUGUCCUGGAUCGGCGACGGGAA
CAACAUCCUGCACUCAAUCAUGAUGAGCGCAGCCAAGUUCGGCAUGCAUCUC
CAAGCCGCUACACCCAAGGGUUAUGAACCGGACGCCUCUGUGACCAAGUUGG
CAGAACAGUACGCCAAGGAGAACGGUACUAAGCUCCUUUUAACCAACGACCC
CCUCGAAGCAGCCCAUGGCGGGAAUGUGCUCAUUACCGAUACCUGGAUUUCG
AUGGGCCAGGAGGAGGAGAAGAAGAAGCGGCUGCAGGCGUUCCAGGGCUACC
AGGUCACCAUGAAAACUGCCAAAGUGGCCGCCUCGGAUUGGACCUUUCUCCA
CUGCCUGCCUCGGAAGCCUGAGGAGGUGGACGACGAAGUGUUCUACUCCCCA
CGGUCCCUCGUGUUCCCCGAGGCCGAAAAUAGGAAGUGGACCAUCAUGGCCG
UGAUGGUGUCCCUCUUGACCGAUUACAGCCCGCAGCUUCAGAAGCCUAAAUU
CUAGCUCGAGCUAGUGACUGACUAGGAUCUGGUUACCACUAAACCAGCCUCA
AGAACACCCGAAUGGAGUCUCUAAGCUACAUAAUACCAACUUACACUUACAA
AAUGUUGUCCCCCAAAAUGUAGCCAUUCGUAUCUGCUCCUAAUAAAAAGAAA
GUUUCUUCACAUUCUAG
>mARM717
(SEQ ID NO: 48)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAAUCUUCGCAUC
CUGUUGAACAACGCCGCCUUCCGCAAUGGUCACAACUUCAUGGUCCGGAACU
UCAGAUGUGGACAGCCUCUCCAAAACAAGGUCCAGCUGAAGGGAAGGGACCU
CUUAACCCUCAAAAACUUUACUGGAGAGGAGAUCAAGUACAUGCUGUGGCUU
AGCGCCGACCUUAAGUUCCGGAUCAAGCAGAAGGGAGAGUACCUCCCGCUGC
UGCAAGGAAAGAGUCUUGGAAUGAUCUUCGAGAAGCGGUCCACCAGAACUCG
CCUCUCCACUGAAACCGGAUUCGCACUCCUGGGUGGACACCCGUGCUUUCUG
ACCACCCAAGACAUCCACCUCGGAGUGAACGAGAGCCUCACGGACACCGCGA
GAGUGCUGUCAUCCAUGGCCGACGCCGUGCUUGCACGGGUCUACAAGCAGUC
CGAUCUGGACACUCUUGCCAAGGAAGCCUCCAUUCCUAUCAUUAACGGUCUG
UCGGAUCUGUACCACCCGAUUCAGAUCCUUGCGGACUACCUCACACUUCAAG
AACACUAUUCAAGCCUAAAGGGUCUGACCCUGUCCUGGAUCGGAGAUGGAAA
CAACAUUCUCCAUUCCAUCAUGAUGAGCGCUGCCAAGUUCGGAAUGCAUCUC
CAAGCAGCGACUCCUAAGGGUUACGAGCCGGACGCCUCAGUGACUAAGCUGG
CCGAGCAGUACGCCAAGGAGAACGGUACCAAACUGUUGCUUACUAACGACCC
GCUUGAAGCGGCCCAUGGAGGAAACGUGCUGAUUACCGACACCUGGAUUUCG
AUGGGACAGGAAGAGGAGAAGAAGAAGCGGCUCCAGGCGUUCCAGGGAUACC
AGGUCACCAUGAAAACGGCCAAAGUGGCCGCUAGCGAUUGGACCUUUCUGCA
CUGCCUCCCGCGCAAGCCUGAAGAAGUGGACGACGAAGUGUUCUACUCCCCU
CGCUCUCUUGUGUUCCCGGAAGCCGAAAACAGGAAGUGGACCAUCAUGGCCG
UGAUGGUGUCCCUCCUGACCGAUUACAGCCCGCAGCUGCAGAAGCCUAAGUU
CUAGCUCGAGCUAGUGACUGACUAGGAUCUGGUUACCACUAAACCAGCCUCA
AGAACACCCGAAUGGAGUCUCUAAGCUACAUAAUACCAACUUACACUUACAA
AAUGUUGUCCCCCAAAAUGUAGCCAUUCGUAUCUGCUCCUAAUAAAAAGAAA
GUUUCUUCACAUUCUAG
>mARM718
(SEQ ID NO: 49)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAAUCUCCGCAUC
CUCCUCAACAACGCCGCGUUUAGAAACGGCCACAACUUCAUGGUCCGGAACU
UCAGAUGUGGCCAGCCGCUUCAGAACAAGGUCCAGCUCAAGGGCCGGGAUCU
UCUGACCCUGAAGAACUUUACUGGCGAAGAAAUCAAGUACAUGCUCUGGCUC
UCCGCCGACUUGAAGUUCCGCAUUAAGCAGAAGGGGGAAUACCUUCCGCUGC
UGCAAGGAAAGUCGCUCGGCAUGAUCUUUGAGAAGCGCUCAACCCGCACCAG
GCUGUCCACUGAAACCGGGUUCGCGCUGCUUGGUGGCCACCCCUGCUUCCUG
ACCACCCAAGACAUUCACCUCGGAGUGAACGAAUCGCUCACUGAUACUGCCC
GGGUGCUGUCGUCGAUGGCCGAUGCAGUGCUGGCCAGGGUGUACAAACAGUC
CGAUCUGGACACUCUGGCCAAGGAGGCGUCCAUCCCUAUUAUCAACGGCCUU
UCCGACCUCUACCACCCGAUUCAGAUCCUUGCCGAUUACCUCACCCUGCAAG
AACACUACUCGUCACUGAAGGGUCUGACCUUGUCCUGGAUCGGCGACGGCAA
CAACAUCCUCCAUUCCAUUAUGAUGUCCGCCGCCAAAUUCGGCAUGCAUCUU
CAAGCCGCAACCCCUAAGGGUUACGAGCCGGACGCUUCCGUGACCAAGCUCG
CCGAGCAGUACGCUAAGGAGAACGGAACCAAGCUUCUGCUGACUAACGACCC
CCUAGAGGCAGCCCACGGGGGCAACGUGCUUAUUACUGACACCUGGAUCUCC
AUGGGACAGGAAGAAGAGAAGAAGAAGCGGUUACAGGCGUUCCAGGGCUAU
CAGGUCACCAUGAAAACCGCCAAGGUCGCUGCCUCGGACUGGACCUUCCUGC
AUUGCCUGCCUCGCAAGCCCGAAGAAGUGGACGACGAGGUGUUCUACUCGCC
ACGGUCCCUUGUGUUCCCUGAGGCCGAGAAUAGAAAGUGGACCAUUAUGGCC
GUGAUGGUGUCCCUUCUCACCGACUACUCGCCGCAACUGCAGAAACCCAAGU
UCUAGCUCGAGCUAGUGACUGACUAGGAUCUGGUUACCACUAAACCAGCCUC
AAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAA
AGUUUCUUCACAUUCUAG
>mARM719
(SEQ ID NO: 50)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAAUCUUCGCAUC
CUCCUCAACAACGCCGCCUUCCGGAACGGUCACAACUUCAUGGUCCGGAACU
UCCGCUGCGGCCAGCCGCUCCAAAACAAAGUGCAGCUUAAGGGCCGCGAUCU
CCUGACCCUGAAGAACUUCACCGGAGAGGAAAUCAAGUACAUGCUGUGGCUC
UCGGCGGACCUGAAGUUUAGGAUUAAGCAGAAGGGGGAGUAUCUGCCGCUGC
UCCAAGGGAAGUCCCUUGGCAUGAUCUUCGAAAAGAGGUCCACCCGGACUCG
GCUCAGCACCGAAACAGGUUUUGCACUUCUGGGGGGCCACCCGUGCUUCCUG
ACGACCCAGGACAUCCAUCUGGGUGUCAACGAGAGUUUGACCGACACUGCCA
GAGUGCUGUCAUCCAUGGCGGACGCGGUGCUCGCGAGAGUGUACAAGCAGUC
CGAUCUUGACACCCUGGCAAAAGAGGCUUCAAUCCCGAUCAUUAACGGACUC
UCGGAUCUGUACCACCCUAUCCAAAUCUUGGCCGACUACCUGACCCUGCAAG
AACACUACAGCUCCCUGAAGGGCCUGACUCUUUCCUGGAUUGGCGAUGGAAA
CAACAUUCUCCAUUCUAUUAUGAUGUCCGCCGCCAAGUUCGGCAUGCACCUU
CAAGCCGCCACCCCGAAGGGCUACGAACCUGACGCCUCCGUGACUAAGCUAG
CCGAACAGUACGCUAAGGAGAACGGCACUAAGCUUCUCCUUACCAACGAUCC
GCUGGAGGCGGCCCAUGGCGGAAAUGUGCUUAUCACCGACACCUGGAUUAGC
AUGGGGCAGGAAGAAGAGAAGAAGAAACGGCUCCAGGCAUUCCAGGGCUACC
AGGUCACCAUGAAAACUGCCAAGGUCGCCGCUAGCGACUGGACCUUCCUCCA
CUGUCUGCCUCGCAAGCCUGAAGAAGUGGACGACGAGGUGUUCUACUCCCCG
CGCUCCCUCGUGUUUCCUGAGGCCGAGAACAGAAAGUGGACCAUCAUGGCCG
UGAUGGUGUCAUUACUUACGGACUACAGCCCGCAGCUGCAGAAGCCGAAGUU
CUAGCUCGAGCUAGUGACUGACUAGGAUCUGGUUACCACUAAACCAGCCUCA
AGAACACCCGAAUGGAGUCUCUAAGCUACAUAAUACCAACUUACACUUACAA
AAUGUUGUCCCCCAAAAUGUAGCCAUUCGUAUCUGCUCCUAAUAAAAAGAAA
GUUUCUUCACAUUCUAG
>mARM720
(SEQ ID NO: 51)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUUAACUUGAGAAUC
CUUCUGAACAACGCCGCUUUCCGCAACGGUCAUAACUUCAUGGUCCGGAACU
UCAGAUGUGGCCAGCCCCUCCAAAACAAAGUGCAGCUGAAGGGCCGGGACCU
UCUUACGCUGAAGAAUUUCACCGGCGAAGAAAUCAAGUACAUGCUCUGGCUG
UCCGCCGAUCUUAAGUUCCGCAUUAAGCAGAAGGGGGAAUACCUCCCGCUGC
UGCAAGGGAAGUCGCUGGGCAUGAUUUUUGAGAAGCGGUCAACUCGCACCCG
CCUGUCCACUGAAACUGGAUUCGCACUGCUCGGUGGCCAUCCCUGCUUCCUG
ACCACCCAAGACAUCCACCUCGGCGUGAACGAGUCCCUGACUGACACCGCCCG
GGUCUUAUCCUCGAUGGCCGAUGCUGUGCUUGCGAGGGUGUACAAGCAGUCC
GACCUCGACACACUCGCGAAGGAGGCCUCCAUCCCCAUCAUCAACGGCCUGU
CCGACCUUUACCACCCAAUUCAGAUCCUCGCCGAUUACCUGACCCUGCAAGA
GCACUACUCGUCGCUCAAGGGGCUUACCCUCUCGUGGAUUGGCGACGGCAAC
AACAUCCUUCACUCCAUCAUGAUGUCGGCAGCGAAGUUCGGCAUGCAUCUGC
AAGCCGCCACGCCUAAGGGUUAUGAACCGGAUGCCUCAGUGACCAAGCUCGC
CGAACAGUACGCGAAAGAGAAUGGAACCAAGCUACUUCUGACCAACGACCCC
CUGGAGGCCGCUCACGGCGGCAACGUCCUCAUUACCGAUACUUGGAUUUCGA
UGGGACAGGAAGAGGAAAAGAAGAAGAGACUGCAGGCGUUCCAGGGAUACC
AGGUCACCAUGAAAACUGCCAAAGUGGCAGCCUCCGACUGGACCUUCCUUCA
CUGCCUGCCGAGGAAGCCUGAAGAGGUGGACGACGAGGUGUUCUACUCCCCG
CGCUCCUUGGUGUUUCCUGAGGCCGAAAACCGGAAGUGGACUAUCAUGGCCG
UGAUGGUGUCCCUCCUCACCGACUACUCGCCGCAACUGCAGAAGCCUAAGUU
CUAGCUCGAGCUAGUGACUGACUAGGAUCUGGUUACCACUAAACCAGCCUCA
AGAACACCCGAAUGGAGUCUCUAAGCUACAUAAUACCAACUUACACUUACAA
AAUGUUGUCCCCCAAAAUGUAGCCAUUCGUAUCUGCUCCUAAUAAAAAGAAA
GUUUCUUCACAUUCUAG
>mARM721
(SEQ ID NO: 52)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGUUAUUCAACCUUAGAAUU
CUCCUUAACAACGCCGCCUUCCGGAAUGGGCAUAACUUUAUGGUCCGCAAUU
UCCGCUGUGGACAGCCUCUGCAAAACAAGGUCCAGCUCAAGGGCCGGGAUCU
GCUGACUCUCAAGAACUUCACUGGGGAAGAAAUCAAGUACAUGCUCUGGCUG
AGCGCCGACCUCAAGUUCCGCAUCAAGCAGAAGGGAGAGUACCUCCCGCUGC
UCCAAGGGAAGUCCCUGGGCAUGAUCUUCGAGAAGAGAUCCACCCGCACCAG
ACUUUCCACUGAGACUGGCUUCGCCUUGCUGGGAGGCCACCCAUGCUUCCUG
ACGACCCAGGACAUUCACCUUGGCGUGAACGAGUCCCUGACUGACACCGCAA
GGGUGUUGUCCUCGAUGGCCGACGCCGUGCUUGCCCGGGUGUACAAGCAGAG
CGAUCUUGACACCCUGGCUAAGGAAGCUUCCAUUCCCAUCAUCAACGGUCUG
AGCGACCUGUACCACCCGAUUCAGAUCCUGGCGGACUACCUAACCCUGCAAG
AGCACUAUAGCUCCCUGAAGGGCCUCACACUUUCAUGGAUCGGCGACGGCAA
CAACAUCCUGCACUCUAUUAUGAUGAGCGCUGCCAAAUUCGGCAUGCACCUC
CAAGCCGCCACGCCUAAAGGCUACGAGCCCGACGCCUCGGUGACCAAGCUUG
CGGAGCAGUACGCGAAGGAAAACGGCACCAAGCUGCUUCUCACCAACGAUCC
UCUGGAAGCGGCCCAUGGUGGCAACGUGCUCAUUACCGACACUUGGAUCUCC
AUGGGACAGGAGGAGGAAAAGAAGAAGCGGCUCCAGGCGUUUCAGGGUUAC
CAGGUCACCAUGAAAACCGCCAAGGUCGCAGCCUCCGACUGGACCUUCCUUC
AUUGCCUUCCGCGCAAGCCCGAAGAAGUGGACGAUGAAGUGUUUUACUCACC
UCGGUCACUCGUGUUCCCGGAAGCAGAGAACAGGAAAUGGACCAUUAUGGCC
GUGAUGGUGUCCCUGCUCACCGAUUACAGUCCGCAACUGCAGAAGCCCAAGU
UCUAGCUCGAGCUAGUGACUGACUAGGAUCUGGUUACCACUAAACCAGCCUC
AAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAA
AGUUUCUUCACAUUCUAG
>mARM722
(SEQ ID NO: 53)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAAUCUCCGCAUC
CUCCUUAACAACGCCGCGUUUAGAAACGGCCACAACUUCAUGGUCCGGAACU
UCAGAUGUGGCCAGCCGCUUCAAAACAAGGUCCAGCUGAAGGGCCGGGAUCU
UCUGACCCUGAAGAACUUUACUGGCGAAGAGAUCAAGUACAUGCUCUGGCUC
UCCGCGGACUUGAAGUUCCGCAUUAAGCAGAAGGGGGAAUACCUUCCGCUGC
UUCAAGGAAAGAGCCUCGGCAUGAUCUUUGAGAAGCGCUCAACCAGGACCCG
CCUUUCUACUGAAACUGGGUUCGCGCUGCUCGGUGGCCACCCCUGCUUCCUG
ACGACCCAGGACAUCCACCUCGGAGUGAACGAAUCCCUCACCGAUACCGCCC
GGGUGUUAUCGAGCAUGGCAGAUGCCGUGCUGGCCAGGGUGUACAAACAGUC
CGAUCUGGACACUCUGGCCAAGGAGGCGUCAAUUCCUAUUAUCAACGGCCUU
AGUGACCUCUACCAUCCGAUUCAAAUCCUGGCCGAUUACCUCACCCUGCAAG
AACACUACAGCUCCCUGAAGGGUCUGACAUUGUCCUGGAUCGGCGACGGCAA
CAACAUUCUCCAUUCCAUCAUGAUGUCCGCCGCAAAAUUCGGCAUGCAUCUU
CAAGCCGCCACGCCUAAGGGUUACGAACCCGACGCUUCCGUGACUAAGCUCG
CCGAGCAGUACGCUAAGGAGAACGGAACCAAGCUGCUGCUGACUAACGACCC
GCUAGAAGCAGCCCACGGGGGCAACGUGCUUAUUACUGACACCUGGAUCUCC
AUGGGCCAGGAGGAAGAGAAAAAGAAGCGGCUGCAGGCGUUCCAGGGAUAU
CAGGUCACCAUGAAAACCGCCAAGGUCGCUGCCUCCGACUGGACCUUCCUGC
ACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCC
GUGAUGGUGUCACUUCUCACCGACUACAGCCCGCAGCUUCAGAAGCCCAAGU
UCUAGCUCGAGCUAGUGACUGACUAGGAUCUGGUUACCACUAAACCAGCCUC
AAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAA
AGUUUCUUCACAUUCUAG
>mARM723
(SEQ ID NO: 54)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAAUCUCCGCAUC
CUCCUUAACAACGCCGCGUUUAGAAACGGCCACAACUUCAUGGUCCGGAACU
UCAGAUGUGGCCAGCCGCUUCAAAACAAGGUCCAGCUUAAGGGCCGGGAUCU
CCUCACCCUUAAAAACUUCACCGGCGAAGAGAUCAAGUACAUGCUCUGGCUC
UCCGCGGACCUUAAGUUCCGCAUUAAGCAGAAGGGGGAAUACCUUCCGCUGC
UUCAAGGAAAGAGCCUCGGCAUGAUCUUUGAGAAGCGCUCAACCAGGACCAG
GCUUUCUACUGAAACUGGGUUCGCGCUUCUCGGCGGUCAUCCCUGCUUCCUC
ACGACCCAAGACAUCCACCUCGGAGUGAACGAAUCCCUCACGGAUACUGCCC
GCGUGCUUUCGAGCAUGGCAGACGCCGUGCUCGCCCGGGUGUACAAACAGUC
CGAUCUCGACACUCUCGCCAAGGAGGCGUCAAUUCCUAUUAUCAACGGUCUU
AGUGACCUUUACCACCCGAUCCAGAUCCUCGCCGAUUACCUCACACUCCAAG
AACACUACAGCUCCCUUAAGGGUCUUACCCUCUCCUGGAUCGGCGACGGCAA
CAACAUUCUCCACUCCAUCAUGAUGUCCGCCGCAAAGUUCGGCAUGCAUCUU
CAAGCCGCCACCCCGAAGGGCUACGAGCCUGAUGCUUCCGUGACUAAGCUCG
CCGAGCAGUACGCUAAGGAGAACGGAACCAAGCUUCUUCUCACUAACGACCC
ACUCGAAGCAGCCCAUGGGGGCAACGUGCUUAUCACUGACACCUGGAUCUCC
AUGGGCCAGGAAGAAGAGAAGAAGAAGCGGCUCCAGGCGUUCCAGGGAUAUC
AGGUCACCAUGAAAACCGCCAAGGUCGCUGCCUCCGACUGGACCUUUCUCCA
CUGCCUCCCUCGCAAACCUGAAGAAGUGGACGACGAGGUGUUCUACUCGCCC
CGGAGCCUCGUGUUCCCCGAGGCCGAGAAUAGAAAGUGGACCAUUAUGGCCG
UGAUGGUGUCACUCCUCACCGACUACAGCCCGCAGCUUCAGAAGCCCAAGUU
CUAGCUCGAGCUAGUGACUGACUAGGAUCUGGUUACCACUAAACCAGCCUCA
AGAACACCCGAAUGGAGUCUCUAAGCUACAUAAUACCAACUUACACUUACAA
AAUGUUGUCCCCCAAAAUGUAGCCAUUCGUAUCUGCUCCUAAUAAAAAGAAA
GUUUCUUCACAUUCUAG
>mARM724
(SEQ ID NO: 55)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAAUCUCCGCAUC
CUCCUUAACAACGCCGCGUUUAGAAACGGACAUAACUUCAUGGUCCGGAACU
UCAGAUGUGGACAGCCGCUUCAAAACAAGGUCCAGCUGAAGGGUCGGGAUCU
UCUGACCCUGAAGAACUUUACCGGAGAAGAGAUCAAGUACAUGCUCUGGCUC
UCCGCGGACUUGAAGUUCCGCAUUAAGCAGAAGGGAGAAUACCUCCCGCUGC
UUCAAGGAAAGAGCCUCGGAAUGAUUUUUGAGAAGCGCUCAACCAGGACCCG
CCUUUCUACUGAAACUGGAUUCGCGCUGCUGGGUGGACACCCCUGCUUCCUG
ACGACCCAGGACAUCCACCUCGGAGUGAACGAAUCCCUCACUGAUACCGCCC
GGGUGUUAUCGAGCAUGGCAGAUGCCGUGCUGGCCAGGGUGUACAAACAGUC
CGAUCUGGACACUCUGGCCAAGGAGGCGUCAAUUCCUAUCAUCAACGGACUU
AGUGACCUCUACCAUCCGAUUCAAAUCCUGGCCGACUACCUCACCCUGCAAG
AACACUACAGCUCCCUGAAGGGUCUGACAUUGUCCUGGAUCGGAGAUGGAAA
CAACAUUCUCCACUCCAUCAUGAUGUCCGCCGCAAAAUUCGGAAUGCAUCUU
CAAGCCGCCACGCCUAAGGGUUACGAACCCGACGCUUCCGUGACUAAGCUCG
CCGAGCAGUACGCUAAGGAGAACGGUACCAAGCUUCUCCUGACCAACGACCC
ACUAGAAGCAGCCCACGGUGGAAACGUGCUUAUUACUGACACUUGGAUCUCC
AUGGGACAGGAGGAAGAGAAAAAGAAGCGGCUGCAGGCGUUCCAGGGAUAU
CAGGUCACCAUGAAAACCGCCAAGGUCGCUGCCUCCGACUGGACCUUCCUGC
ACUGCCUGCCUCGCAAGCCUGAAGAAGUGGACGACGAGGUGUUCUACUCGCC
GCGGAGCCUCGUGUUCCCCGAGGCCGAGAAUAGAAAGUGGACCAUCAUGGCC
GUGAUGGUGUCACUGCUCACCGACUACAGCCCGCAGCUUCAGAAGCCCAAGU
UCUAGCUCGAGCUAGUGACUGACUAGGAUCUGGUUACCACUAAACCAGCCUC
AAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAA
AGUUUCUUCACAUUCUAG
>mARM725
(SEQ ID NO: 56)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAACCUCCGCAUU
CUCCUCAACAACGCUGCCUUCCGGAAUGGACAUAACUUCAUGGUCCGGAACU
UCAGAUGCGGACAGCCGCUUCAGAACAAGGUCCAGCUUAAGGGGAGAGAUCU
CCUUACCCUCAAAAACUUCACUGGCGAAGAAAUCAAGUACAUGCUCUGGCUU
AGUGCGGAUCUCAAGUUCCGCAUCAAGCAGAAGGGAGAAUACCUCCCGCUCC
UUCAAGGAAAGAGCCUCGGCAUGAUUUUUGAGAAGAGGUCCACCAGAACUCG
CCUUUCAACCGAGACUGGGUUCGCCCUGCUUGGCGGUCACCCCUGCUUCCUC
ACUACCCAAGACAUCCACCUCGGCGUGAACGAGAGCCUUACCGACACCGCCC
GCGUGCUCUCCUCAAUGGCCGACGCUGUGCUCGCCCGGGUGUACAAGCAGUC
CGACCUUGAUACUCUCGCCAAGGAGGCCUCCAUCCCAAUUAUCAACGGGCUC
UCUGAUCUCUACCACCCUAUCCAAAUCCUCGCGGACUACCUCACCCUCCAAGA
GCACUAUAGCUCGCUCAAGGGCCUCACCCUUUCCUGGAUUGGCGACGGCAAC
AACAUUCUUCACUCGAUCAUGAUGUCCGCCGCCAAGUUCGGCAUGCAUCUCC
AAGCCGCGACCCCCAAGGGCUACGAGCCUGACGCAUCCGUGACCAAGCUCGC
CGAGCAGUACGCGAAGGAAAAUGGCACCAAGCUUCUUCUCACCAACGACCCC
CUUGAGGCCGCUCAUGGCGGCAACGUGCUCAUCACUGACACUUGGAUCAGCA
UGGGCCAGGAGGAGGAAAAGAAGAAGCGCCUUCAGGCAUUCCAGGGUUACCA
GGUCACCAUGAAAACCGCCAAAGUGGCCGCCUCCGACUGGACCUUUCUUCAC
UGUCUCCCGCGGAAGCCUGAAGAAGUGGAUGACGAAGUGUUUUACUCCCCUC
GGUCACUCGUGUUCCCGGAAGCAGAAAACAGGAAGUGGACCAUUAUGGCGGU
CAUGGUGUCCCUCCUCACCGACUACAGCCCGCAGCUUCAGAAACCCAAGUUC
UAGCUCGAGCUAGUGACUGACUAGGAUCUGGUUACCACUAAACCAGCCUCAA
GAACACCCGAAUGGAGUCUCUAAGCUACAUAAUACCAACUUACACUUACAAA
AUGUUGUCCCCCAAAAUGUAGCCAUUCGUAUCUGCUCCUAAUAAAAAGAAAG
UUUCUUCACAUUCUAG
>mARM726
(SEQ ID NO: 57)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAAUCUCCGCAUC
CUCCUUAACAACGCAGCGUUUAGAAACGGUCACAACUUCAUGGUCCGGAACU
UCCGCUGUGGACAGCCGCUUCAAAACAAGGUCCAGCUGAAGGGUCGGGACCU
UCUGACCCUGAAGAACUUUACUGGAGAAGAGAUCAAGUACAUGCUUUGGCUG
UCCGCGGACUUGAAGUUCCGCAUUAAGCAGAAGGGAGAAUACCUUCCGCUGC
UCCAAGGAAAGAGCCUGGGAAUGAUCUUUGAGAAGCGCUCAACCAGGACCCG
CCUUUCUACUGAAACUGGAUUCGCGCUGCUGGGUGGUCACCCUUGCUUCCUG
ACGACCCAGGACAUUCACCUCGGAGUGAACGAGUCCCUCACUGAUACCGCCA
GAGUGUUAUCGAGCAUGGCAGAUGCCGUGCUGGCUAGGGUGUACAAACAGUC
CGAUCUGGACACCCUGGCCAAGGAGGCAUCAAUUCCUAUUAUCAACGGACUU
AGUGACCUCUACCAUCCGAUUCAAAUCCUGGCCGAUUACCUCACCCUGCAAG
AACACUACAGCUCCCUGAAGGGUCUGACAUUGUCCUGGAUCGGAGAUGGAAA
CAACAUUCUCCAUUCCAUCAUGAUGUCCGCGGCCAAGUUCGGAAUGCAUCUC
CAAGCCGCCACGCCGAAAGGAUACGAGCCGGACGCUUCCGUGACUAAGCUCG
CCGAGCAGUACGCUAAGGAGAACGGAACCAAGCUUCUGCUGACUAACGACCC
GCUAGAAGCCGCCCACGGUGGAAACGUGCUUAUUACUGACACCUGGAUCUCC
AUGGGACAGGAAGAAGAGAAAAAGAAGCGGCUGCAGGCGUUCCAGGGAUAU
CAGGUCACCAUGAAAACCGCCAAGGUCGCCGCCUCCGACUGGACCUUCCUUC
ACUGCCUGCCUCGGAAGCCUGAAGAAGUGGACGACGAGGUGUUCUACUCGCC
GCGGAGCCUCGUGUUCCCUGAGGCCGAGAAUAGAAAGUGGACCAUCAUGGCC
GUGAUGGUGUCACUCCUCACCGACUACAGCCCGCAGCUUCAGAAGCCUAAGU
UCUAGCUCGAGCUAGUGACUGACUAGGAUCUGGUUACCACUAAACCAGCCUC
AAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAA
AGUUUCUUCACAUUCUAG
>mARM727
(SEQ ID NO: 58)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAAUCUCCGCAUU
CUCCUCAACAACGCAGCCUUUAGAAACGGCCACAACUUCAUGGUCCGGAACU
UCAGAUGUGGCCAGCCGCUUCAGAACAAGGUCCAGCUCAAGGGCCGGGACCU
CCUCACCCUCAAAAACUUUACCGGCGAAGAGAUCAAGUACAUGCUCUGGCUU
UCGGCCGACCUUAAGUUCCGCAUCAAGCAGAAGGGGGAAUACCUUCCGCUGC
UUCAAGGAAAGUCCCUCGGCAUGAUCUUUGAAAAGCGCUCGACCAGGACCCG
CCUUUCCACUGAAACCGGGUUCGCGCUUCUCGGUGGCCACCCCUGCUUCCUC
ACCACCCAAGACAUUCACCUCGGAGUGAACGAAUCCCUUACCGAUACCGCAA
GAGUGCUUUCGUCGAUGGCCGAUGCCGUGCUUGCGCGGGUGUACAAGCAGUC
AGAUCUCGACACUCUCGCCAAGGAGGCGUCCAUUCCUAUUAUCAACGGCCUU
UCCGACCUUUACCACCCGAUUCAGAUCCUCGCCGAUUACCUCACCCUGCAAG
AGCACUACUCGUCACUCAAGGGUCUUACCCUCUCCUGGAUCGGCGACGGAAA
CAACAUCCUCCAUUCGAUCAUGAUGUCCGCCGCCAAAUUCGGCAUGCACCUC
CAAGCCGCGACCCCGAAGGGUUACGAGCCCGACGCUUCCGUGACCAAGCUCG
CCGAACAGUACGCUAAGGAAAACGGCACCAAGCUCCUCCUCACUAACGACCC
UCUCGAAGCAGCCCAUGGGGGCAACGUGCUCAUUACUGACACUUGGAUCUCG
AUGGGCCAGGAAGAGGAGAAAAAGAAGCGGCUUCAGGCGUUCCAGGGAUAU
CAGGUCACCAUGAAAACCGCCAAGGUCGCUGCCUCGGACUGGACCUUCCUUC
ACUGCCUUCCGCGCAAGCCUGAAGAGGUGGACGAUGAGGUGUUCUACUCCCC
ACGGUCCCUUGUGUUCCCCGAGGCCGAGAAUAGGAAGUGGACCAUCAUGGCC
GUGAUGGUGUCGCUCCUCACUGACUACUCCCCGCAACUUCAGAAGCCUAAGU
UCUAGCUCGAGCUAGUGACUGACUAGGAUCUGGUUACCACUAAACCAGCCUC
AAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAA
AGUUUCUUCACAUUCUAG
>mARM728
(SEQ ID NO: 59)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUGUUUAAUCUGAGAAUA
CUUCUAAACAACGCCGCCUUCCGGAAUGGCCAUAACUUUAUGGUUCGGAAUU
UCCGCUGCGGCCAGCCGCUGCAGAACAAGGUCCAGCUGAAGGGAAGAGACUU
GCUGACCCUCAAGAACUUCACCGGAGAAGAAAUCAAGUAUAUGCUGUGGCUG
UCCGCCGACCUGAAAUUCCGCAUCAAGCAGAAGGGCGAAUAUCUGCCGCUGU
UGCAAGGGAAGUCCCUGGGGAUGAUCUUCGAGAAGAGGUCCACCAGAACACG
GCUUUCAACCGAAACCGGGUUUGCACUGCUGGGUGGACACCCCUGUUUUCUG
ACCACUCAAGAUAUCCACCUGGGCGUGAACGAGUCCCUUACCGACACUGCUA
GGGUGUUGUCCAGCAUGGCCGAUGCCGUCCUGGCUCGCGUGUACAAGCAGUC
CGACCUGGAUACCCUGGCAAAGGAAGCGUCCAUUCCCAUUAUCAACGGGCUG
UCCGACCUGUACCAUCCGAUUCAAAUCCUGGCGGACUACCUGACUCUGCAAG
AGCAUUACAGCAGCUUGAAGGGGCUUACUCUCUCGUGGAUCGGCGACGGGAA
CAACAUCCUGCACUCCAUCAUGAUGUCCGCCGCCAAGUUCGGGAUGCAUUUG
CAAGCUGCGACCCCGAAAGGUUACGAGCCCGAUGCUAGCGUAACUAAGCUUG
CCGAACAGUACGCCAAAGAGAAUGGUACAAAACUGCUUCUGACUAACGACCC
GCUGGAAGCAGCCCACGGCGGGAACGUGCUGAUAACCGACACCUGGAUUUCA
AUGGGGCAGGAGGAAGAGAAGAAGAAGCGACUGCAGGCGUUCCAAGGCUAU
CAGGUUACCAUGAAAACCGCCAAAGUGGCAGCCAGCGAUUGGACUUUCCUGC
ACUGUCUGCCGCGGAAGCCCGAGGAAGUUGAUGACGAAGUAUUCUACUCACC
CCGGAGCCUCGUGUUCCCCGAGGCCGAAAACCGGAAGUGGACUAUUAUGGCC
GUGAUGGUGUCGCUGUUGACCGACUACAGCCCGCAACUGCAGAAGCCGAAGU
UUUAGCUCGAGCUAGUGACUGACUAGGAUCUGGUUACCACUAAACCAGCCUC
AAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAA
AGUUUCUUCACAUUCUAG
>mARM729
(SEQ ID NO: 60)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAACCUGAGGAUC
CUUUUGAACAACGCCGCCUUUCGCAACGGCCACAACUUUAUGGUCCGCAAUU
UCCGCUGCGGGCAGCCGCUGCAGAACAAGGUCCAGCUGAAGGGCCGGGAUCU
GCUGACCCUGAAGAACUUCACCGGGGAGGAAAUCAAGUACAUGCUUUGGCUC
UCCGCCGAUCUGAAGUUCAGAAUCAAGCAGAAGGGAGAGUACCUCCCGUUGC
UGCAAGGAAAGUCACUCGGAAUGAUUUUCGAAAAGAGAAGCACUAGGACCCG
CCUCUCAACUGAAACCGGGUUCGCGCUGCUCGGGGGCCAUCCGUGUUUCCUG
ACUACCCAAGACAUCCACCUGGGAGUGAACGAGUCGCUGACCGACACCGCAC
GCGUGCUGUCAUCCAUGGCGGACGCAGUGCUUGCCCGGGUGUACAAGCAGUC
GGACCUGGACACUCUUGCCAAGGAGGCAUCAAUCCCCAUCAUUAACGGACUG
UCCGAUCUCUACCACCCGAUUCAGAUCCUGGCUGACUACCUAACCCUGCAAG
AGCACUACUCAAGCCUGAAGGGGCUGACCCUGUCGUGGAUCGGGGACGGCAA
CAACAUUCUGCACUCCAUCAUGAUGUCGGCGGCUAAGUUCGGGAUGCAUUUG
CAAGCGGCAACUCCGAAGGGUUAUGAACCCGACGCCUCCGUGACCAAGCUGG
CCGAACAGUACGCCAAGGAAAACGGAACCAAGUUGCUGCUGACUAAUGAUCC
CCUGGAGGCGGCCCACGGGGGGAACGUGCUGAUAACCGAUACCUGGAUCUCC
AUGGGGCAGGAAGAAGAGAAGAAAAAGCGGCUGCAGGCAUUCCAGGGAUAC
CAGGUCACCAUGAAAACCGCAAAAGUGGCAGCCAGCGACUGGACUUUCCUCC
AUUGCCUGCCGCGAAAGCCGGAGGAGGUCGAUGACGAGGUGUUCUACUCCCC
GCGGUCGCUGGUGUUCCCGGAGGCGGAAAACCGGAAGUGGACCAUUAUGGCC
GUGAUGGUGUCACUCCUGACUGACUACAGCCCGCAACUGCAGAAGCCGAAGU
UCUAGCUCGAGCUAGUGACUGACUAGGAUCUGGUUACCACUAAACCAGCCUC
AAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAA
AGUUUCUUCACAUUCUAG
>mARM1787
(SEQ ID NO: 61)
CUUAAGGGGGCGCUGCCUACGGAGGUGGCAGCCAUCUCCUUCUCGGCAUCAA
GCUUACCAUGGUGCCCCAGGCCCUGCUCUUGGUCCCGCUGCUGGUGUUCCCC
CUCUGCUUCGGCAAGUUCCCCAUCUACACCAUCCCCGACAAGCUGGGGCCGU
GGAGCCCCAUCGACAUCCACCACCUGUCCUGCCCCAACAACCUCGUGGUCGAG
GACGAGGGCUGCACCAACCUGAGCGGGUUCUCCUACAUGCUUUUCAAUCUCC
GCAUCCUCCUUAACAACGCCGCGUUUAGAAACGGCCACAACUUCAUGGUCCG
GAACUUCAGAUGUGGCCAGCCGCUUCAAAACAAGGUCCAGCUGAAGGGCCGG
GAUCUUCUGACCCUGAAGAACUUUACUGGCGAAGAGAUCAAGUACAUGCUCU
GGCUCUCCGCGGACUUGAAGUUCCGCAUUAAGCAGAAGGGGGAAUACCUUCC
GCUGCUUCAAGGAAAGAGCCUCGGCAUGAUCUUUGAGAAGCGCUCAACCAGG
ACCCGCCUUUCUACUGAAACUGGGUUCGCGCUGCUCGGUGGCCACCCCUGCU
UCCUGACGACCCAGGACAUCCACCUCGGAGUGAACGAAUCCCUCACCGAUAC
CGCCCGGGUGUUAUCGAGCAUGGCAGAUGCCGUGCUGGCCAGGGUGUACAAA
CAGUCCGAUCUGGACACUCUGGCCAAGGAGGCGUCAAUUCCUAUUAUCAACG
GCCUUAGUGACCUCUACCAUCCGAUUCAGAUCCUGGCCGAUUACCUCACCCU
GCAAGAACACUACAGCUCCCUGAAGGGUCUGACAUUGUCCUGGAUCGGCGAC
GGCAACAACAUUCUCCAUUCCAUCAUGAUGUCCGCCGCAAAAUUCGGCAUGC
AUCUUCAAGCCGCCACGCCGAAGGGUUACGAGCCCGACGCUUCCGUGACUAA
GCUCGCCGAGCAGUACGCUAAGGAGAACGGAACCAAGCUUCUGCUGACUAAC
GACCCACUAGAAGCAGCCCACGGGGGCAACGUGCUUAUUACUGACACCUGGA
UCUCCAUGGGCCAGGAAGAAGAGAAAAAGAAGCGGCUGCAGGCGUUCCAGGG
AUAUCAGGUCACCAUGAAAACCGCCAAGGUCGCUGCCUCCGACUGGACCUUC
CUGCACUGCCUGCCUCGCAAGCCUGAAGAAGUGGACGACGAGGUGUUCUACU
CGCCACGGAGCCUCGUGUUCCCCGAGGCCGAGAAUAGAAAGUGGACCAUCAU
GGCCGUGAUGGUGUCACUGCUCACCGACUACAGCCCGCAGCUUCAGAAGCCC
AAGUUCUAGAUAAGUGAAUGCAAGGCUGGCCGGAAGCCCUUGCCUGAAAGCA
AGAUUUCAGCCUGGAAGAGGGCAAAGUGGACGGGAGUGGACAGGAGUGGAU
GCGAUAAGAUGUGGUUUGAAGCUGAUGGGUGCCAGCCCUGCAUUGCUGAGUC
AAUCAAUAAAGAGCUUUCUUUUGACCCAUUCUAGAUCUAG
>mARM1788
(SEQ ID NO: 62)
AGGAAACUUAAGAUUAUUACAUCAAAACAAAAAGCCGCCAAUGCUGUUCAAC
CUGCGCAUCCUGCUGAACAACGCCGCCUUCCGCAACGGCCACAACUUCAUGG
UGCGCAACUUCCGCUGCGGCCAGCCCCUGCAGAACAAGGUGCAGCUGAAGGG
CCGCGACCUGCUGACCCUGAAGAACUUCACCGGCGAGGAGAUCAAGUACAUG
CUGUGGCUGAGCGCCGACCUGAAGUUCCGCAUCAAGCAGAAGGGCGAGUACC
UGCCCCUGCUGCAGGGCAAGAGCCUGGGCAUGAUCUUCGAGAAGCGCAGCAC
CCGCACCCGCCUGAGCACCGAGACAGGCCUGGCCCUGCUGGGCGGCCACCCCU
GCUUCCUGACCACCCAGGACAUCCACCUGGGCGUGAACGAGAGCCUGACCGA
CACCGCCCGCGUGCUGAGCAGCAUGGCCGACGCCGUGCUGGCCCGCGUGUAC
AAGCAGAGCGACCUGGACACCCUGGCCAAGGAGGCCAGCAUCCCCAUCAUCA
ACGGCCUGAGCGACCUGUACCACCCCAUCCAGAUCCUGGCCGACUACCUGACC
CUGCAGGAGCACUACAGCAGCCUGAAGGGCCUGACCCUGAGCUGGAUCGGCG
ACGGCAACAACAUCCUGCACAGCAUCAUGAUGAGCGCCGCCAAGUUCGGCAU
GCACCUGCAGGCCGCCACCCCCAAGGGCUACGAGCCCGACGCCAGCGUGACCA
AGCUGGCCGAGCAGUACGCCAAGGAGAACGGCACCAAGCUGCUGCUGACCAA
CGACCCCCUGGAGGCCGCCCACGGCGGCAACGUGCUGAUCACCGACACCUGG
AUCAGCAUGGGCCAGGAGGAGGAGAAGAAGAAGCGCCUGCAGGCCUUCCAGG
GCUACCAGGUGACCAUGAAGACCGCCAAGGUGGCCGCCAGCGACUGGACCUU
CCUGCACUGCCUGCCCCGCAAGCCCGAGGAGGUGGACGACGAGGUGUUCUAC
AGCCCCCGCAGCCUGGUGUUCCCCGAGGCCGAGAACCGCAAGUGGACCAUCA
UGGCCGUGAUGGUGAGCCUGCUGACCGACUACAGCCCCCAGCUGCAGAAGCC
CAAGUUCUGAACGCCGAAGCCUGCAGCCAUGCGACCCCACGCCACCCCGUGCC
UCCUGCCUCCGCGCAGCCUGCAGCGGGAGACCCUGUCCCCGCCCCAGCCGUCC
UCCUGGGGUGGACCCUAGUUUAAUAAAGAUUCACCAAGUUUCACGCAAAAAA
AAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAA
AAAAAAAAAAAAA
>mARM1789
(SEQ ID NO: 63)
CUUAAGGGGGCGCUGCCUACGGAGGUGGCAGCCAUCUCCUUCUCGGCAUCAA
GCUUACCAUGGUGCCCCAGGCCCUGCUCUUGGUCCCGCUGCUGGUGUUCCCC
CUCUGCUUCGGCAAGUUCCCCAUCUACACCAUCCCCGACAAGCUGGGGCCGU
GGAGCCCCAUCGACAUCCACCACCUGUCCUGCCCCAACAACCUCGUGGUCGAG
GACGAGGGCUGCACCAACCUGAGCGGGUUCUCCUACAUGCUGUUCAACCUGC
GCAUCCUGCUGAACAACGCCGCCUUCCGCAACGGCCACAACUUCAUGGUGCG
CAACUUCCGCUGCGGCCAGCCCCUGCAGAACAAGGUGCAGCUGAAGGGCCGC
GACCUGCUGACCCUGAAGAACUUCACCGGCGAGGAGAUCAAGUACAUGCUGU
GGCUGAGCGCCGACCUGAAGUUCCGCAUCAAGCAGAAGGGCGAGUACCUGCC
CCUGCUGCAGGGCAAGAGCCUGGGCAUGAUCUUCGAGAAGCGCAGCACCCGC
ACCCGCCUGAGCACCGAGACAGGCCUGGCCCUGCUGGGCGGCCACCCCUGCUU
CCUGACCACCCAGGACAUCCACCUGGGCGUGAACGAGAGCCUGACCGACACC
GCCCGCGUGCUGAGCAGCAUGGCCGACGCCGUGCUGGCCCGCGUGUACAAGC
AGAGCGACCUGGACACCCUGGCCAAGGAGGCCAGCAUCCCCAUCAUCAACGG
CCUGAGCGACCUGUACCACCCCAUCCAGAUCCUGGCCGACUACCUGACCCUGC
AGGAGCACUACAGCAGCCUGAAGGGCCUGACCCUGAGCUGGAUCGGCGACGG
CAACAACAUCCUGCACAGCAUCAUGAUGAGCGCCGCCAAGUUCGGCAUGCAC
CUGCAGGCCGCCACCCCCAAGGGCUACGAGCCCGACGCCAGCGUGACCAAGCU
GGCCGAGCAGUACGCCAAGGAGAACGGCACCAAGCUGCUGCUGACCAACGAC
CCCCUGGAGGCCGCCCACGGCGGCAACGUGCUGAUCACCGACACCUGGAUCA
GCAUGGGCCAGGAGGAGGAGAAGAAGAAGCGCCUGCAGGCCUUCCAGGGCUA
CCAGGUGACCAUGAAGACCGCCAAGGUGGCCGCCAGCGACUGGACCUUCCUG
CACUGCCUGCCCCGCAAGCCCGAGGAGGUGGACGACGAGGUGUUCUACAGCC
CCCGCAGCCUGGUGUUCCCCGAGGCCGAGAACCGCAAGUGGACCAUCAUGGC
CGUGAUGGUGAGCCUGCUGACCGACUACAGCCCCCAGCUGCAGAAGCCCAAG
UUCUGAAUAAGUGAUGCAAGGCUGGCCGGAAGCCCUUGCCUGAAAGCAAGAU
UUCAGCCUGGAAGAGGGCAAAGUGGACGGGAGUGGACAGGAGUGGAUGCGA
UAAGAUGUGGUUUGAAGCUGAUGGGUGCCAGCCCUGCAUUGCUGAGUCAAUC
AAUAAAGAGCUUUCUUUUGACCCAUUCUAGAUCUAG
>mARM1790
(SEQ ID NO: 64)
AGGAAACUUAAGAUUAUUACAUCAAAACAAAAAGCCGCCAAUGCUUUUCAAU
CUCCGCAUCCUCCUUAACAACGCCGCGUUUAGAAACGGCCACAACUUCAUGG
UCCGGAACUUCAGAUGUGGCCAGCCGCUUCAAAACAAGGUCCAGCUGAAGGG
CCGGGAUCUUCUGACCCUGAAGAACUUUACUGGCGAAGAGAUCAAGUACAUG
CUCUGGCUCUCCGCGGACUUGAAGUUCCGCAUUAAGCAGAAGGGGGAAUACC
UUCCGCUGCUUCAAGGAAAGAGCCUCGGCAUGAUCUUUGAGAAGCGCUCAAC
CAGGACCCGCCUUUCUACUGAAACUGGGUUCGCGCUGCUCGGUGGCCACCCC
UGCUUCCUGACGACCCAGGACAUCCACCUCGGAGUGAACGAAUCCCUCACCG
AUACCGCCCGGGUGUUAUCGAGCAUGGCAGAUGCCGUGCUGGCCAGGGUGUA
CAAACAGUCCGAUCUGGACACUCUGGCCAAGGAGGCGUCAAUUCCUAUUAUC
AACGGCCUUAGUGACCUCUACCAUCCGAUUCAGAUCCUGGCCGAUUACCUCA
CCCUGCAAGAACACUACAGCUCCCUGAAGGGUCUGACAUUGUCCUGGAUCGG
CGACGGCAACAACAUUCUCCAUUCCAUCAUGAUGUCCGCCGCAAAAUUCGGC
AUGCAUCUUCAAGCCGCCACGCCGAAGGGUUACGAGCCCGACGCUUCCGUGA
CUAAGCUCGCCGAGCAGUACGCUAAGGAGAACGGAACCAAGCUUCUGCUGAC
UAACGACCCACUAGAAGCAGCCCACGGGGGCAACGUGCUUAUUACUGACACC
UGGAUCUCCAUGGGCCAGGAAGAAGAGAAAAAGAAGCGGCUGCAGGCGUUCC
AGGGAUAUCAGGUCACCAUGAAAACCGCCAAGGUCGCUGCCUCCGACUGGAC
CUUCCUGCACUGCCUGCCUCGCAAGCCUGAAGAAGUGGACGACGAGGUGUUC
UACUCGCCACGGAGCCUCGUGUUCCCCGAGGCCGAGAAUAGAAAGUGGACCA
UCAUGGCCGUGAUGGUGUCACUGCUCACCGACUACAGCCCGCAGCUUCAGAA
GCCCAAGUUCUAGACGCCGAAGCCUGCAGCCAUGCGACCCCACGCCACCCCGU
GCCUCCUGCCUCCGCGCAGCCUGCAGCGGGAGACCCUGUCCCCGCCCCAGCCG
UCCUCCUGGGGUGGACCCUAGUUUAAUAAAGAUUCACCAAGUUUCACGCAAA
AAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAA
AAAAAAAAAAAAAAAA
>mARM1791
(SEQ ID NO: 65)
CUUAAGGGGGCGCUGCCUACGGAGGUGGCAGCCAUCUCCUUCUCGGCAUCAA
GCUUACCAUGGUGCCCCAGGCCCUGCUCUUGGUCCCGCUGCUGGUGUUCCCC
CUCUGCUUCGGCAAGUUCCCCAUCUACACCAUCCCCGACAAGCUGGGGCCGU
GGAGCCCCAUCGACAUCCACCACCUGUCCUGCCCCAACAACCUCGUGGUCGAG
GACGAGGGCUGCACCAACCUGAGCGGGUUCUCCUACAUGCUUUUCAACCUGA
GAAUCCUCUUGAACAAUGCUGCUUUUCGGAAUGGCCACAACUUUAUGGUUCG
GAACUUCCGUUGCGGCCAGCCUUUACAAAACAAGGUCCAGCUGAAGGGCCGG
GAUUUGCUCACACUAAAGAACUUUACUGGAGAAGAGAUCAAGUACAUGCUA
UGGCUGUCGGCCGACCUGAAGUUCCGUAUCAAGCAGAAGGGAGAAUACCUUC
CGCUGCUUCAAGGAAAGAGCCUCGGCAUGAUCUUUGAGAAGCGCUCAACCAG
GACCCGCCUUUCUACUGAAACUGGGUUCGCGCUGCUCGGUGGCCACCCCUGC
UUCCUGACGACCCAGGACAUCCACCUCGGAGUGAACGAAUCCCUCACCGAUA
CCGCCCGGGUGUUAUCGAGCAUGGCAGAUGCCGUGCUGGCCAGGGUGUACAA
ACAGUCCGAUCUCGAUACCUUGGCAAAGGAGGCUUCCAUUCCCAUCAUCAAC
GGCCUGAGCGACCUGUACCACCCAAUCCAAAUCCUGGCUGACUACCUGACCC
UGCAAGAGCACUACAGCAGCCUGAAGGGUCUGACCCUGUCAUGGAUUGGCGA
UGGAAACAAUAUUCUGCACUCCAUCAUGAUGUCCGCCGCGAAGUUCGGAAUG
CAUCUGCAAGCCGCCACUCCAAAAGGAUACGAACCGGAUGCAUCCGUGACCA
AGUUGGCGGAACAGUACGCGAAGGAGAACGGAACCAAGCUCCUGCUGACUAA
CGACCCGCUCGAGGCUGCGCAUGGGGGUAACGUGCUGAUUACGGACACCUGG
AUCUCCAUGGGGCAGGAGGAAGAGAAGAAGAAGAGACUGCAGGCAUUCCAG
GGGUACCAGGUCACCAUGAAAACCGCAAAAGUGGCAGCUUCGGACUGGACUU
UCCUGCAUUGCCUGCCGAGGAAGCCGGAGGAAGUCGACGACGAAGUGUUCUA
CUCGCCUCGGUCCCUGGUGUUCCCCGAGGCCGAAAACCGGAAGUGGACCAUC
AUGGCCGUGAUGGUGUCCUUGCUGACUGACUAUAGCCCGCAGCUGCAGAAGC
CUAAGUUCUAGAUAAGUGAUGCAAGGCUGGCCGGAAGCCCUUGCCUGAAAGC
AAGAUUUCAGCCUGGAAGAGGGCAAAGUGGACGGGAGUGGACAGGAGUGGA
UGCGAUAAGAUGUGGUUUGAAGCUGAUGGGUGCCAGCCCUGCAUUGCUGAG
UCAAUCAAUAAAGAGCUUUCUUUUGACCCAUUCUAGAUCUAG
>mARM1792
(SEQ ID NO: 66)
UGAGUGUCGUACAGCCUCCAGGCCCCCCCCUCCCGGGAGAGCCAUAGUGGUC
UGCGGAACCGGUGAGUACACCGGAAUUGCCGGGAAGACUGGGUCCUUUCUUG
GAUAAACCCACUCUAUGCCCGGCCAUUUGGGCGUGCCCCCGCAAGACUGCUA
GCCGAGUAGUGUUGGGUUGCGAUGCUGUUCAACCUGCGCAUCCUGCUGAACA
ACGCCGCCUUCCGCAACGGCCACAACUUCAUGGUGCGCAACUUCCGCUGCGG
CCAGCCCCUGCAGAACAAGGUGCAGCUGAAGGGCCGCGACCUGCUGACCCUG
AAGAACUUCACCGGCGAGGAGAUCAAGUACAUGCUGUGGCUGAGCGCCGACC
UGAAGUUCCGCAUCAAGCAGAAGGGCGAGUACCUGCCCCUGCUGCAGGGCAA
GAGCCUGGGCAUGAUCUUCGAGAAGCGCAGCACCCGCACCCGCCUGAGCACC
GAGACAGGCCUGGCCCUGCUGGGCGGCCACCCCUGCUUCCUGACCACCCAGG
ACAUCCACCUGGGCGUGAACGAGAGCCUGACCGACACCGCCCGCGUGCUGAG
CAGCAUGGCCGACGCCGUGCUGGCCCGCGUGUACAAGCAGAGCGACCUGGAC
ACCCUGGCCAAGGAGGCCAGCAUCCCCAUCAUCAACGGCCUGAGCGACCUGU
ACCACCCCAUCCAGAUCCUGGCCGACUACCUGACCCUGCAGGAGCACUACAGC
AGCCUGAAGGGCCUGACCCUGAGCUGGAUCGGCGACGGCAACAACAUCCUGC
ACAGCAUCAUGAUGAGCGCCGCCAAGUUCGGCAUGCACCUGCAGGCCGCCAC
CCCCAAGGGCUACGAGCCCGACGCCAGCGUGACCAAGCUGGCCGAGCAGUAC
GCCAAGGAGAACGGCACCAAGCUGCUGCUGACCAACGACCCCCUGGAGGCCG
CCCACGGCGGCAACGUGCUGAUCACCGACACCUGGAUCAGCAUGGGCCAGGA
GGAGGAGAAGAAGAAGCGCCUGCAGGCCUUCCAGGGCUACCAGGUGACCAUG
AAGACCGCCAAGGUGGCCGCCAGCGACUGGACCUUCCUGCACUGCCUGCCCC
GCAAGCCCGAGGAGGUGGACGACGAGGUGUUCUACAGCCCCCGCAGCCUGGU
GUUCCCCGAGGCCGAGAACCGCAAGUGGACCAUCAUGGCCGUGAUGGUGAGC
CUGCUGACCGACUACAGCCCCCAGCUGCAGAAGCCCAAGUUCUGAAUAAGUG
AUAGAGCGGCAAACCCUAGCUACACUCCAUAGCUAGUUUCUUUUUUUUUUGU
UUUUUUUUUUUUUUUUUUUUUUUUUUUUUUUUUUUUUUCCUUUCUUUUCCU
UCUUUUUUUCCUCUUUUCUUGGUGGCUCCAUCUUAGCCCUAGUCACGGCUAG
CUGUGAAAGGUCCGUGAGCCGCAUGACUGCAGAGAGUGCCGUAACUGGCCUC
UCUGCAGAUCAUGUUCUAG
>mARM1793
(SEQ ID NO: 67)
AGGAAACUUAAGAUUAUUACAUCAAAACAAAAAGCCGCCAAUGCUUUUCAAU
CUCCGCAUCCUCCUUAACAACGCCGCGUUUAGAAACGGCCACAACUUCAUGG
UCCGGAACUUCAGAUGUGGCCAGCCGCUUCAAAACAAGGUCCAGCUGAAGGG
CCGGGAUCUUCUGACCCUGAAGAACUUUACUGGCGAAGAGAUCAAGUACAUG
CUCUGGCUCUCCGCGGACUUGAAGUUCCGCAUUAAGCAGAAGGGGGAAUACC
UUCCGCUGCUUCAAGGAAAGAGCCUCGGCAUGAUCUUUGAGAAGCGCUCAAC
CAGGACCCGCCUUUCUACUGAAACUGGGUUCGCGCUGCUCGGUGGCCACCCC
UGCUUCCUGACGACCCAGGACAUCCACCUCGGAGUGAACGAAUCCCUCACCG
AUACCGCCCGGGUGUUAUCGAGCAUGGCAGAUGCCGUGCUGGCCAGGGUGUA
CAAACAGUCCGAUCUGGACACUCUGGCCAAGGAGGCGUCAAUUCCUAUUAUC
AACGGCCUUAGUGACCUCUACCAUCCGAUUCAGAUCCUGGCCGAUUACCUCA
CCCUGCAAGAACACUACAGCUCCCUGAAGGGUCUGACAUUGUCCUGGAUCGG
CGACGGCAACAACAUUCUCCAUUCCAUCAUGAUGUCCGCCGCAAAAUUCGGC
AUGCAUCUUCAAGCCGCCACGCCGAAGGGUUACGAGCCCGACGCUUCCGUGA
CUAAGCUCGCCGAGCAGUACGCUAAGGAGAACGGAACCAAGCUUCUGCUGAC
UAACGACCCACUAGAAGCAGCCCACGGGGGCAACGUGCUUAUUACUGACACC
UGGAUCUCCAUGGGCCAGGAAGAAGAGAAAAAGAAGCGGCUGCAGGCGUUCC
AGGGAUAUCAGGUCACCAUGAAAACCGCCAAGGUCGCUGCCUCCGACUGGAC
CUUCCUGCACUGCCUGCCUCGCAAGCCUGAAGAAGUGGACGACGAGGUGUUC
UACUCGCCACGGAGCCUCGUGUUCCCCGAGGCCGAGAAUAGAAAGUGGACCA
UCAUGGCCGUGAUGGUGUCACUGCUCACCGACUACAGCCCGCAGCUUCAGAA
GCCCAAGUUCUAGGCUGGAGCCUCGGUAGCCGUUCCUCCUGCCCGCUGGGCC
UCCCAACGGGCCCUCCUCCCCUCCUUGCACCGGCCCUUCCUGGUCUUUGAAUA
AAGUCUGAGUGGGCAGCAAAAAAAAAAAAAAAAAAAAAAAAAAAAAAAAAA
AAAAAAAAAAAAAAAAAAAAAAAAAAAAAAAAAAAAAAAAAAAAAAAAAAA
AAAAAAAAAAAAAAAAAAAAAAAAAAAAAAAAAAAA
>mARM1794
(SEQ ID NO: 68)
AGGAAACUUAAGAUUAUUACAUCAAAACAAAAAGCCGCCAAUGCUGUUCAAC
CUGCGCAUCCUGCUGAACAACGCCGCCUUCCGCAACGGCCACAACUUCAUGG
UGCGCAACUUCCGCUGCGGCCAGCCCCUGCAGAACAAGGUGCAGCUGAAGGG
CCGCGACCUGCUGACCCUGAAGAACUUCACCGGCGAGGAGAUCAAGUACAUG
CUGUGGCUGAGCGCCGACCUGAAGUUCCGCAUCAAGCAGAAGGGCGAGUACC
UGCCCCUGCUGCAGGGCAAGAGCCUGGGCAUGAUCUUCGAGAAGCGCAGCAC
CCGCACCCGCCUGAGCACCGAGACAGGCCUGGCCCUGCUGGGCGGCCACCCCU
GCUUCCUGACCACCCAGGACAUCCACCUGGGCGUGAACGAGAGCCUGACCGA
CACCGCCCGCGUGCUGAGCAGCAUGGCCGACGCCGUGCUGGCCCGCGUGUAC
AAGCAGAGCGACCUGGACACCCUGGCCAAGGAGGCCAGCAUCCCCAUCAUCA
ACGGCCUGAGCGACCUGUACCACCCCAUCCAGAUCCUGGCCGACUACCUGACC
CUGCAGGAGCACUACAGCAGCCUGAAGGGCCUGACCCUGAGCUGGAUCGGCG
ACGGCAACAACAUCCUGCACAGCAUCAUGAUGAGCGCCGCCAAGUUCGGCAU
GCACCUGCAGGCCGCCACCCCCAAGGGCUACGAGCCCGACGCCAGCGUGACCA
AGCUGGCCGAGCAGUACGCCAAGGAGAACGGCACCAAGCUGCUGCUGACCAA
CGACCCCCUGGAGGCCGCCCACGGCGGCAACGUGCUGAUCACCGACACCUGG
AUCAGCAUGGGCCAGGAGGAGGAGAAGAAGAAGCGCCUGCAGGCCUUCCAGG
GCUACCAGGUGACCAUGAAGACCGCCAAGGUGGCCGCCAGCGACUGGACCUU
CCUGCACUGCCUGCCCCGCAAGCCCGAGGAGGUGGACGACGAGGUGUUCUAC
AGCCCCCGCAGCCUGGUGUUCCCCGAGGCCGAGAACCGCAAGUGGACCAUCA
UGGCCGUGAUGGUGAGCCUGCUGACCGACUACAGCCCCCAGCUGCAGAAGCC
CAAGUUCUGAGCUGGAGCCUCGGUAGCCGUUCCUCCUGCCCGCUGGGCCUCC
CAACGGGCCCUCCUCCCCUCCUUGCACCGGCCCUUCCUGGUCUUUGAAUAAA
GUCUGAGUGGGCAGCAAAAAAAAAAAAAAAAAAAAAAAAAAAAAAAAAAAA
AAAAAAAAAAAAAAAAAAAAAAAAAAAAAAAAAAAAAAAAAAAAAAAAAAA
AAAAAAAAAAAAAAAAAAAAAAAAAAAAAAAAAA
>mARM1795
(SEQ ID NO: 69)
AGGAAACUUAAGAUUAUUACAUCAAAACAAAAAGCCGCCAAUGCUUUUCAAC
CUGAGAAUCCUCUUGAACAAUGCUGCUUUUCGGAAUGGCCACAACUUUAUGG
UUCGGAACUUCCGUUGCGGCCAGCCUUUACAAAACAAGGUCCAGCUGAAGGG
CCGGGAUUUGCUCACACUAAAGAACUUUACUGGAGAAGAGAUCAAGUACAUG
CUAUGGCUGUCGGCCGACCUGAAGUUCCGUAUCAAGCAGAAGGGAGAAUACC
UUCCGCUGCUUCAAGGAAAGAGCCUCGGCAUGAUCUUUGAGAAGCGCUCAAC
CAGGACCCGCCUUUCUACUGAAACUGGGUUCGCGCUGCUCGGUGGCCACCCC
UGCUUCCUGACGACCCAGGACAUCCACCUCGGAGUGAACGAAUCCCUCACCG
AUACCGCCCGGGUGUUAUCGAGCAUGGCAGAUGCCGUGCUGGCCAGGGUGUA
CAAACAGUCCGAUCUCGAUACCUUGGCAAAGGAGGCUUCCAUUCCCAUCAUC
AACGGCCUGAGCGACCUGUACCACCCAAUCCAAAUCCUGGCUGACUACCUGA
CCCUGCAAGAGCACUACAGCAGCCUGAAGGGUCUGACCCUGUCAUGGAUUGG
CGAUGGAAACAAUAUUCUGCACUCCAUCAUGAUGUCCGCCGCGAAGUUCGGA
AUGCAUCUGCAAGCCGCCACUCCAAAAGGAUACGAACCGGAUGCAUCCGUGA
CCAAGUUGGCGGAACAGUACGCGAAGGAGAACGGAACCAAGCUCCUGCUGAC
UAACGACCCGCUCGAGGCUGCGCAUGGGGGUAACGUGCUGAUUACGGACACC
UGGAUCUCCAUGGGGCAGGAGGAAGAGAAGAAGAAGAGACUGCAGGCAUUC
CAGGGGUACCAGGUCACCAUGAAAACCGCAAAAGUGGCAGCUUCGGACUGGA
CUUUCCUGCAUUGCCUGCCGAGGAAGCCGGAGGAAGUCGACGACGAAGUGUU
CUACUCGCCUCGGUCCCUGGUGUUCCCCGAGGCCGAAAACCGGAAGUGGACC
AUCAUGGCCGUGAUGGUGUCCUUGCUGACUGACUAUAGCCCGCAGCUGCAGA
AGCCUAAGUUCUAGGCUGGAGCCUCGGUAGCCGUUCCUCCUGCCCGCUGGGC
CUCCCAACGGGCCCUCCUCCCCUCCUUGCACCGGCCCUUCCUGGUCUUUGAAU
AAAGUCUGAGUGGGCAGCAAAAAAAAAAAAAAAAAAAAAAAAAAAAAAAAA
AAAAAAAAAAAAAAAAAAAAAAAAAAAAAAAAAAAAAAAAAAAAAAAAAAA
AAAAAAAAAAAAAAAAAAAAAAAAAAAAAAAAAAAAA
>mARM1796
(SEQ ID NO: 70)
AGGAAACUUAAGAAUUAUUGGUUAAAGAAGUAUAUUAGUGCUAAUUUCCCU
CCGUUUGUCCUAGCUUUUCUCUUCUGUCAACCCCACACGCCUUUGGCACAAU
GCUUUUCAAUCUCCGCAUCCUCCUUAACAACGCCGCGUUUAGAAACGGCCAC
AACUUCAUGGUCCGGAACUUCAGAUGUGGCCAGCCGCUUCAAAACAAGGUCC
AGCUGAAGGGCCGGGAUCUUCUGACCCUGAAGAACUUUACUGGCGAAGAGAU
CAAGUACAUGCUCUGGCUCUCCGCGGACUUGAAGUUCCGCAUUAAGCAGAAG
GGGGAAUACCUUCCGCUGCUUCAAGGAAAGAGCCUCGGCAUGAUCUUUGAGA
AGCGCUCAACCAGGACCCGCCUUUCUACUGAAACUGGGUUCGCGCUGCUCGG
UGGCCACCCCUGCUUCCUGACGACCCAGGACAUCCACCUCGGAGUGAACGAA
UCCCUCACCGAUACCGCCCGGGUGUUAUCGAGCAUGGCAGAUGCCGUGCUGG
CCAGGGUGUACAAACAGUCCGAUCUGGACACUCUGGCCAAGGAGGCGUCAAU
UCCUAUUAUCAACGGCCUUAGUGACCUCUACCAUCCGAUUCAGAUCCUGGCC
GAUUACCUCACCCUGCAAGAACACUACAGCUCCCUGAAGGGUCUGACAUUGU
CCUGGAUCGGCGACGGCAACAACAUUCUCCAUUCCAUCAUGAUGUCCGCCGC
AAAAUUCGGCAUGCAUCUUCAAGCCGCCACGCCGAAGGGUUACGAGCCCGAC
GCUUCCGUGACUAAGCUCGCCGAGCAGUACGCUAAGGAGAACGGAACCAAGC
UUCUGCUGACUAACGACCCACUAGAAGCAGCCCACGGGGGCAACGUGCUUAU
UACUGACACCUGGAUCUCCAUGGGCCAGGAAGAAGAGAAAAAGAAGCGGCUG
CAGGCGUUCCAGGGAUAUCAGGUCACCAUGAAAACCGCCAAGGUCGCUGCCU
CCGACUGGACCUUCCUGCACUGCCUGCCUCGCAAGCCUGAAGAAGUGGACGA
CGAGGUGUUCUACUCGCCACGGAGCCUCGUGUUCCCCGAGGCCGAGAAUAGA
AAGUGGACCAUCAUGGCCGUGAUGGUGUCACUGCUCACCGACUACAGCCCGC
AGCUUCAGAAGCCCAAGUUCUAGCUCGAGACACAUCACAACCACAACCUUCU
CAGGCUACCCUGAGAAAAAAAGACAUGAAGACUCAGGACUCAUCUUUUCUGU
UGGUGUAAAAUCAACACCCUAAGGAACACAAAUUUCUUUAAACAUUUGACUU
CUUGUCUCUGUGCUGCAAUUAAUAAAAAAUGGAAAGAAUCUAUCUAG
>mARM1797
(SEQ ID NO: 71)
AGGAAACUUAAGAAUUAUUGGUUAAAGAAGUAUAUUAGUGCUAAUUUCCCU
CCGUUUGUCCUAGCUUUUCUCUUCUGUCAACCCCACACGCCUUUGGCACAAU
GCUGUUCAACCUGCGCAUCCUGCUGAACAACGCCGCCUUCCGCAACGGCCAC
AACUUCAUGGUGCGCAACUUCCGCUGCGGCCAGCCCCUGCAGAACAAGGUGC
AGCUGAAGGGCCGCGACCUGCUGACCCUGAAGAACUUCACCGGCGAGGAGAU
CAAGUACAUGCUGUGGCUGAGCGCCGACCUGAAGUUCCGCAUCAAGCAGAAG
GGCGAGUACCUGCCCCUGCUGCAGGGCAAGAGCCUGGGCAUGAUCUUCGAGA
AGCGCAGCACCCGCACCCGCCUGAGCACCGAGACAGGCCUGGCCCUGCUGGGC
GGCCACCCCUGCUUCCUGACCACCCAGGACAUCCACCUGGGCGUGAACGAGA
GCCUGACCGACACCGCCCGCGUGCUGAGCAGCAUGGCCGACGCCGUGCUGGC
CCGCGUGUACAAGCAGAGCGACCUGGACACCCUGGCCAAGGAGGCCAGCAUC
CCCAUCAUCAACGGCCUGAGCGACCUGUACCACCCCAUCCAGAUCCUGGCCGA
CUACCUGACCCUGCAGGAGCACUACAGCAGCCUGAAGGGCCUGACCCUGAGC
UGGAUCGGCGACGGCAACAACAUCCUGCACAGCAUCAUGAUGAGCGCCGCCA
AGUUCGGCAUGCACCUGCAGGCCGCCACCCCCAAGGGCUACGAGCCCGACGCC
AGCGUGACCAAGCUGGCCGAGCAGUACGCCAAGGAGAACGGCACCAAGCUGC
UGCUGACCAACGACCCCCUGGAGGCCGCCCACGGCGGCAACGUGCUGAUCAC
CGACACCUGGAUCAGCAUGGGCCAGGAGGAGGAGAAGAAGAAGCGCCUGCAG
GCCUUCCAGGGCUACCAGGUGACCAUGAAGACCGCCAAGGUGGCCGCCAGCG
ACUGGACCUUCCUGCACUGCCUGCCCCGCAAGCCCGAGGAGGUGGACGACGA
GGUGUUCUACAGCCCCCGCAGCCUGGUGUUCCCCGAGGCCGAGAACCGCAAG
UGGACCAUCAUGGCCGUGAUGGUGAGCCUGCUGACCGACUACAGCCCCCAGC
UGCAGAAGCCCAAGUUCUGACUCGAGACACAUCACAACCACAACCUUCUCAG
GCUACCCUGAGAAAAAAAGACAUGAAGACUCAGGACUCAUCUUUUCUGUUGG
UGUAAAAUCAACACCCUAAGGAACACAAAUUUCUUUAAACAUUUGACUUCUU
GUCUCUGUGCUGCAAUUAAUAAAAAAUGGAAAGAAUCUAUCUAG
>mARM1798
(SEQ ID NO: 72)
AGGAAACUUAAGAAUUAUUGGUUAAAGAAGUAUAUUAGUGCUAAUUUCCCU
CCGUUUGUCCUAGCUUUUCUCUUCUGUCAACCCCACACGCCUUUGGCACAAU
GCUUUUCAACCUGAGAAUCCUCUUGAACAAUGCUGCUUUUCGGAAUGGCCAC
AACUUUAUGGUUCGGAACUUCCGUUGCGGCCAGCCUUUACAAAACAAGGUCC
AGCUGAAGGGCCGGGAUUUGCUCACACUAAAGAACUUUACUGGAGAAGAGA
UCAAGUACAUGCUAUGGCUGUCGGCCGACCUGAAGUUCCGUAUCAAGCAGAA
GGGAGAAUACCUUCCGCUGCUUCAAGGAAAGAGCCUCGGCAUGAUCUUUGAG
AAGCGCUCAACCAGGACCCGCCUUUCUACUGAAACUGGGUUCGCGCUGCUCG
GUGGCCACCCCUGCUUCCUGACGACCCAGGACAUCCACCUCGGAGUGAACGA
AUCCCUCACCGAUACCGCCCGGGUGUUAUCGAGCAUGGCAGAUGCCGUGCUG
GCCAGGGUGUACAAACAGUCCGAUCUCGAUACCUUGGCAAAGGAGGCUUCCA
UUCCCAUCAUCAACGGCCUGAGCGACCUGUACCACCCAAUCCAAAUCCUGGC
UGACUACCUGACCCUGCAAGAGCACUACAGCAGCCUGAAGGGUCUGACCCUG
UCAUGGAUUGGCGAUGGAAACAAUAUUCUGCACUCCAUCAUGAUGUCCGCCG
CGAAGUUCGGAAUGCAUCUGCAAGCCGCCACUCCAAAAGGAUACGAACCGGA
UGCAUCCGUGACCAAGUUGGCGGAACAGUACGCGAAGGAGAACGGAACCAAG
CUCCUGCUGACUAACGACCCGCUCGAGGCUGCGCAUGGGGGUAACGUGCUGA
UUACGGACACCUGGAUCUCCAUGGGGCAGGAGGAAGAGAAGAAGAAGAGAC
UGCAGGCAUUCCAGGGGUACCAGGUCACCAUGAAAACCGCAAAAGUGGCAGC
UUCGGACUGGACUUUCCUGCAUUGCCUGCCGAGGAAGCCGGAGGAAGUCGAC
GACGAAGUGUUCUACUCGCCUCGGUCCCUGGUGUUCCCCGAGGCCGAAAACC
GGAAGUGGACCAUCAUGGCCGUGAUGGUGUCCUUGCUGACUGACUAUAGCCC
GCAGCUGCAGAAGCCUAAGUUCUAGCUCGAGACACAUCACAACCACAACCUU
CUCAGGCUACCCUGAGAAAAAAAGACAUGAAGACUCAGGACUCAUCUUUUCU
GUUGGUGUAAAAUCAACACCCUAAGGAACACAAAUUUCUUUAAACAUUUGAC
UUCUUGUCUCUGUGCUGCAAUUAAUAAAAAAUGGAAAGAAUCUAUCUAG
>mARM1799
(SEQ ID NO: 73)
AUUAUUACAUCAAAACAAAAAGCCGCCACCAUGCUGUUCAACCUGCGCAUCC
UGCUGAACAACGCCGCCUUCCGCAACGGCCACAACUUCAUGGUGCGCAACUU
CCGCUGCGGCCAGCCCCUGCAGAACAAGGUGCAGCUGAAGGGCCGCGACCUG
CUGACCCUGAAGAACUUCACCGGCGAGGAGAUCAAGUACAUGCUGUGGCUGA
GCGCCGACCUGAAGUUCCGCAUCAAGCAGAAGGGCGAGUACCUGCCCCUGCU
GCAGGGCAAGAGCCUGGGCAUGAUCUUCGAGAAGCGCAGCACCCGCACCCGC
CUGAGCACCGAGACAGGCUUCGCCCUGCUGGGCGGCCACCCCUGCUUCCUGA
CCACCCAGGACAUCCACCUGGGCGUGAACGAGAGCCUGACCGACACCGCCCGC
GUGCUGAGCAGCAUGGCCGACGCCGUGCUGGCCCGCGUGUACAAGCAGAGCG
ACCUGGACACCCUGGCCAAGGAGGCCAGCAUCCCCAUCAUCAACGGCCUGAG
CGACCUGUACCACCCCAUCCAGAUCCUGGCCGACUACCUGACCCUGCAGGAGC
ACUACAGCAGCCUGAAGGGCCUGACCCUGAGCUGGAUCGGCGACGGCAACAA
CAUCCUGCACAGCAUCAUGAUGAGCGCCGCCAAGUUCGGCAUGCACCUGCAG
GCCGCCACCCCCAAGGGCUACGAGCCCGACGCCAGCGUGACCAAGCUGGCCGA
GCAGUACGCCAAGGAGAACGGCACCAAGCUGCUGCUGACCAACGACCCCCUG
GAGGCCGCCCACGGCGGCAACGUGCUGAUCACCGACACCUGGAUCAGCAUGG
GCCAGGAGGAGGAGAAGAAGAAGCGCCUGCAGGCCUUCCAGGGCUACCAGGU
GACCAUGAAGACCGCCAAGGUGGCCGCCAGCGACUGGACCUUCCUGCACUGC
CUGCCCCGCAAGCCCGAGGAGGUGGACGACGAGGUGUUCUACAGCCCCCGCA
GCCUGGUGUUCCCCGAGGCCGAGAACCGCAAGUGGACCAUCAUGGCCGUGAU
GGUGAGCCUGCUGACCGACUACAGCCCCCAGCUGCAGAAGCCCAAGUUCUGA
GGUCUCUAGUAAUGAGCUGGAGCCUCGGUAGCCGUUCCUCCUGCCCGCUGGG
CCUCCCAACGGGCCCUCCUCCCCUCCUUGCACCGGCCCUUCCUGGUCUUUGAA
UAAAGUCUGAGUGGGCAUCUAG
>mARM1800
(SEQ ID NO: 74)
AGGAAACUUAAGAAUUAUUGGUUAAAGAAGUAUAUUAGUGCUAAUUUCCCU
CCGUUUGUCCUAGCUUUUCUCUUCUGUCAACCCCACACGCCUUUGGCACAAU
GCUGUUCAACCUGCGCAUCCUGCUGAACAACGCCGCCUUCCGCAACGGCCAC
AACUUCAUGGUGCGCAACUUCCGCUGCGGCCAGCCCCUGCAGAACAAGGUGC
AGCUGAAGGGCCGCGACCUGCUGACCCUGAAGAACUUCACCGGCGAGGAGAU
CAAGUACAUGCUGUGGCUGAGCGCCGACCUGAAGUUCCGCAUCAAGCAGAAG
GGCGAGUACCUGCCCCUGCUGCAGGGCAAGAGCCUGGGCAUGAUCUUCGAGA
AGCGCAGCACCCGCACCCGCCUGAGCACCGAGACAGGCUUCGCCCUGCUGGGC
GGCCACCCCUGCUUCCUGACCACCCAGGACAUCCACCUGGGCGUGAACGAGA
GCCUGACCGACACCGCCCGCGUGCUGAGCAGCAUGGCCGACGCCGUGCUGGC
CCGCGUGUACAAGCAGAGCGACCUGGACACCCUGGCCAAGGAGGCCAGCAUC
CCCAUCAUCAACGGCCUGAGCGACCUGUACCACCCCAUCCAGAUCCUGGCCGA
CUACCUGACCCUGCAGGAGCACUACAGCAGCCUGAAGGGCCUGACCCUGAGC
UGGAUCGGCGACGGCAACAACAUCCUGCACAGCAUCAUGAUGAGCGCCGCCA
AGUUCGGCAUGCACCUGCAGGCCGCCACCCCCAAGGGCUACGAGCCCGACGCC
AGCGUGACCAAGCUGGCCGAGCAGUACGCCAAGGAGAACGGCACCAAGCUGC
UGCUGACCAACGACCCCCUGGAGGCCGCCCACGGCGGCAACGUGCUGAUCAC
CGACACCUGGAUCAGCAUGGGCCAGGAGGAGGAGAAGAAGAAGCGCCUGCAG
GCCUUCCAGGGCUACCAGGUGACCAUGAAGACCGCCAAGGUGGCCGCCAGCG
ACUGGACCUUCCUGCACUGCCUGCCCCGCAAGCCCGAGGAGGUGGACGACGA
GGUGUUCUACAGCCCCCGCAGCCUGGUGUUCCCCGAGGCCGAGAACCGCAAG
UGGACCAUCAUGGCCGUGAUGGUGAGCCUGCUGACCGACUACAGCCCCCAGC
UGCAGAAGCCCAAGUUCUGACUCGAGACACAUCACAACCACAACCUUCUCAG
GCUACCCUGAGAAAAAAAGACAUGAAGACUCAGGACUCAUCUUUUCUGUUGG
UGUAAAAUCAACACCCUAAGGAACACAAAUUUCUUUAAACAUUUGACUUCUU
GUCUCUGUGCUGCAAUUAAUAAAAAAUGGAAAGAAUCUAUCUAG
>mARM1801
(SEQ ID NO: 75)
AGGAAACUUAAGAUUAUUACAUCAAAACAAAAAGCCGCCAAUGCUGUUCAAC
CUGCGCAUCCUGCUGAACAACGCCGCCUUCCGCAACGGCCACAACUUCAUGG
UGCGCAACUUCCGCUGCGGCCAGCCCCUGCAGAACAAGGUGCAGCUGAAGGG
CCGCGACCUGCUGACCCUGAAGAACUUCACCGGCGAGGAGAUCAAGUACAUG
CUGUGGCUGAGCGCCGACCUGAAGUUCCGCAUCAAGCAGAAGGGCGAGUACC
UGCCCCUGCUGCAGGGCAAGAGCCUGGGCAUGAUCUUCGAGAAGCGCAGCAC
CCGCACCCGCCUGAGCACCGAGACAGGCUUCGCCCUGCUGGGCGGCCACCCCU
GCUUCCUGACCACCCAGGACAUCCACCUGGGCGUGAACGAGAGCCUGACCGA
CACCGCCCGCGUGCUGAGCAGCAUGGCCGACGCCGUGCUGGCCCGCGUGUAC
AAGCAGAGCGACCUGGACACCCUGGCCAAGGAGGCCAGCAUCCCCAUCAUCA
ACGGCCUGAGCGACCUGUACCACCCCAUCCAGAUCCUGGCCGACUACCUGACC
CUGCAGGAGCACUACAGCAGCCUGAAGGGCCUGACCCUGAGCUGGAUCGGCG
ACGGCAACAACAUCCUGCACAGCAUCAUGAUGAGCGCCGCCAAGUUCGGCAU
GCACCUGCAGGCCGCCACCCCCAAGGGCUACGAGCCCGACGCCAGCGUGACCA
AGCUGGCCGAGCAGUACGCCAAGGAGAACGGCACCAAGCUGCUGCUGACCAA
CGACCCCCUGGAGGCCGCCCACGGCGGCAACGUGCUGAUCACCGACACCUGG
AUCAGCAUGGGCCAGGAGGAGGAGAAGAAGAAGCGCCUGCAGGCCUUCCAGG
GCUACCAGGUGACCAUGAAGACCGCCAAGGUGGCCGCCAGCGACUGGACCUU
CCUGCACUGCCUGCCCCGCAAGCCCGAGGAGGUGGACGACGAGGUGUUCUAC
AGCCCCCGCAGCCUGGUGUUCCCCGAGGCCGAGAACCGCAAGUGGACCAUCA
UGGCCGUGAUGGUGAGCCUGCUGACCGACUACAGCCCCCAGCUGCAGAAGCC
CAAGUUCUGAACGCCGAAGCCUGCAGCCAUGCGACCCCACGCCACCCCGUGCC
UCCUGCCUCCGCGCAGCCUGCAGCGGGAGACCCUGUCCCCGCCCCAGCCGUCC
UCCUGGGGUGGACCCUAGUUUAAUAAAGAUUCACCAAGUUUCACGCAAAAAA
AAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAA
AAAAAAAAAAAAA
>mARM1802
(SEQ ID NO: 76)
CUUAAGGGGGCGCUGCCUACGGAGGUGGCAGCCAUCUCCUUCUCGGCAUCAA
GCUUACCAUGGUGCCCCAGGCCCUGCUCUUGGUCCCGCUGCUGGUGUUCCCC
CUCUGCUUCGGCAAGUUCCCCAUCUACACCAUCCCCGACAAGCUGGGGCCGU
GGAGCCCCAUCGACAUCCACCACCUGUCCUGCCCCAACAACCUCGUGGUCGAG
GACGAGGGCUGCACCAACCUGAGCGGGUUCUCCUACAUGCUGUUCAACCUGC
GCAUCCUGCUGAACAACGCCGCCUUCCGCAACGGCCACAACUUCAUGGUGCG
CAACUUCCGCUGCGGCCAGCCCCUGCAGAACAAGGUGCAGCUGAAGGGCCGC
GACCUGCUGACCCUGAAGAACUUCACCGGCGAGGAGAUCAAGUACAUGCUGU
GGCUGAGCGCCGACCUGAAGUUCCGCAUCAAGCAGAAGGGCGAGUACCUGCC
CCUGCUGCAGGGCAAGAGCCUGGGCAUGAUCUUCGAGAAGCGCAGCACCCGC
ACCCGCCUGAGCACCGAGACAGGCUUCGCCCUGCUGGGCGGCCACCCCUGCUU
CCUGACCACCCAGGACAUCCACCUGGGCGUGAACGAGAGCCUGACCGACACC
GCCCGCGUGCUGAGCAGCAUGGCCGACGCCGUGCUGGCCCGCGUGUACAAGC
AGAGCGACCUGGACACCCUGGCCAAGGAGGCCAGCAUCCCCAUCAUCAACGG
CCUGAGCGACCUGUACCACCCCAUCCAGAUCCUGGCCGACUACCUGACCCUGC
AGGAGCACUACAGCAGCCUGAAGGGCCUGACCCUGAGCUGGAUCGGCGACGG
CAACAACAUCCUGCACAGCAUCAUGAUGAGCGCCGCCAAGUUCGGCAUGCAC
CUGCAGGCCGCCACCCCCAAGGGCUACGAGCCCGACGCCAGCGUGACCAAGCU
GGCCGAGCAGUACGCCAAGGAGAACGGCACCAAGCUGCUGCUGACCAACGAC
CCCCUGGAGGCCGCCCACGGCGGCAACGUGCUGAUCACCGACACCUGGAUCA
GCAUGGGCCAGGAGGAGGAGAAGAAGAAGCGCCUGCAGGCCUUCCAGGGCUA
CCAGGUGACCAUGAAGACCGCCAAGGUGGCCGCCAGCGACUGGACCUUCCUG
CACUGCCUGCCCCGCAAGCCCGAGGAGGUGGACGACGAGGUGUUCUACAGCC
CCCGCAGCCUGGUGUUCCCCGAGGCCGAGAACCGCAAGUGGACCAUCAUGGC
CGUGAUGGUGAGCCUGCUGACCGACUACAGCCCCCAGCUGCAGAAGCCCAAG
UUCUGAAUAAGUGAAUGCAAGGCUGGCCGGAAGCCCUUGCCUGAAAGCAAGA
UUUCAGCCUGGAAGAGGGCAAAGUGGACGGGAGUGGACAGGAGUGGAUGCG
AUAAGAUGUGGUUUGAAGCUGAUGGGUGCCAGCCCUGCAUUGCUGAGUCAA
UCAAUAAAGAGCUUUCUUUUGACCCAUUCUAGAUCUAG
>mARM1803
(SEQ ID NO: 77)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGGGCGUCUUCAACCUGCGG
AUCCUGCUGAACAACGCCGCCUUCCGGAACGGCCACAACUUCAUGGUCCGCA
ACUUCAGAUGCGGCCAGCCCCUGCAGAACAAGGUGCAGCUGAAGGGCCGGGA
CCUGCUGACCCUGAAGAACUUCACCGGCGAAGAGAUCAAGUACAUGCUGUGG
CUGAGCGCCGACCUGAAGUUCCGGAUCAAGCAGAAGGGCGAGUACCUGCCCC
UGCUGCAAGGCAAGAGCCUGGGCAUGAUCUUCGAGAAGCGGAGCACCCGGAC
CCGGCUGAGCACCGAGACAGGCUUUGCCCUGCUGGGAGGCCACCCCUGCUUU
CUGACCACCCAGGACAUCCACCUGGGCGUGAACGAGAGCCUGACCGACACCG
CCAGAGUGCUGAGCAGCAUGGCCGACGCCGUGCUGGCCCGGGUGUACAAGCA
GAGCGACCUGGACACCCUGGCCAAAGAGGCCAGCAUCCCCAUCAUCAACGGC
CUGAGCGACCUGUACCACCCCAUCCAGAUCCUGGCCGACUACCUGACCCUGCA
GGAACACUACAGCUCCCUGAAGGGCCUGACCCUGAGCUGGAUCGGCGACGGC
AACAACAUCCUGCACAGCAUCAUGAUGAGCGCCGCCAAGUUCGGCAUGCAUC
UGCAGGCCGCCACCCCCAAGGGCUACGAGCCUGAUGCCAGCGUGACCAAGCU
GGCCGAGCAGUACGCCAAAGAGAACGGCACCAAGCUGCUGCUGACCAACGAC
CCCCUGGAAGCCGCCCACGGCGGCAACGUGCUGAUCACCGACACCUGGAUCA
GCAUGGGCCAGGAAGAGGAAAAGAAGAAGCGGCUGCAGGCCUUCCAGGGCUA
CCAGGUCACAAUGAAGACCGCCAAGGUGGCCGCCAGCGACUGGACCUUCCUG
CACUGCCUGCCCCGGAAGCCCGAAGAGGUGGACGACGAGGUGUUCUACAGCC
CCCGGUCCCUGGUGUUCCCCGAGGCCGAGAACCGGAAGUGGACCAUUAUGGC
CGUGAUGGUGUCCCUGCUGACCGACUACUCCCCCCAGCUGCAGAAGCCCAAG
UUCUAGAUAAGUGAACUCGAGCUAGUGACUGACUAGGAUCUGGUUACCACUA
AACCAGCCUCAAGAACACCCGAAUGGAGUCUCUAAGCUACAUAAUACCAACU
UACACUUACAAAAUGUUGUCCCCCAAAAUGUAGCCAUUCGUAUCUGCUCCUA
AUAAAAAGAAAGUUUCUUCACAUUCUAG
>mARM1804
(SEQ ID NO: 78)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGGGCGUCUUCAACCUGCGG
AUCCUGCUGAACAACGCCGCCUUCCGGAACGGCCACAACUUCAUGGUCCGCA
ACUUCAGAUGCGGCCAGCCCCUGCAGAACAGGGUGCAGCUGAAGGGCCGGGA
CCUGCUGACCCUGAAGAACUUCACCGGCGAAGAGAUCAGGUACAUGCUGUGG
CUGAGCGCCGACCUGAAGUUCCGGAUCAAGCAGAAGGGCGAGUACCUGCCCC
UGCUGCAAGGCAAGAGCCUGGGCAUGAUCUUCGAGAAGCGGAGCACCCGGAC
CCGGCUGAGCACCGAGACAGGCUUUGCCCUGCUGGGAGGCCACCCCUGCUUU
CUGACCACCCAGGACAUCCACCUGGGCGUGAACGAGAGCCUGACCGACACCG
CCAGAGUGCUGAGCAGCAUGGCCGACGCCGUGCUGGCCCGGGUGUACAAGCA
GAGCGACCUGGACACCCUGGCCAAAGAGGCCAGCAUCCCCAUCAUCAACGGC
CUGAGCGACCUGUACCACCCCAUCCAGAUCCUGGCCGACUACCUGACCCUGCA
GGAACACUACAGCUCCCUGAAGGGCCUGACCCUGAGCUGGAUCGGCGACGGC
AACAACAUCCUGCACAGCAUCAUGAUGAGCGCCGCCAAGUUCGGCAUGCAUC
UGCAGGCCGCCACCCCCAAGGGCUACGAGCCUGAUGCCAGCGUGACCAAGCU
GGCCGAGCAGUACGCCAAAGAGAACGGCACCAAGCUGCUGCUGACCAACGAC
CCCCUGGAAGCCGCCCACGGCGGCAACGUGCUGAUCACCGACACCUGGAUCA
GCAUGGGCCAGGAAGAGGAAAAGAAGAAGCGGCUGCAGGCCUUCCAGGGCUA
CCAGGUCACAAUGAAGACCGCCAAGGUGGCCGCCAGCGACUGGACCUUCCUG
CACUGCCUGCCCCGGAAGCCCGAAGAGGUGGACGACGAGGUGUUCUACAGCC
CCCGGUCCCUGGUGUUCCCCGAGGCCGAGAACCGGAAGUGGACCAUUAUGGC
CGUGAUGGUGUCCCUGCUGACCGACUACUCCCCCCAGCUGCAGAAGCCCAAG
UUCUAGAUAAGUGAACUCGAGCUAGUGACUGACUAGGAUCUGGUUACCACUA
AACCAGCCUCAAGAACACCCGAAUGGAGUCUCUAAGCUACAUAAUACCAACU
UACACUUACAAAAUGUUGUCCCCCAAAAUGUAGCCAUUCGUAUCUGCUCCUA
AUAAAAAGAAAGUUUCUUCACAUUCUAG
>mARM1805
(SEQ ID NO: 79)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUGGUCUUCAACCUGCGG
AUCCUGCUGAACAACGCCGCCUUCCGGAACGGCCACAACUUCAUGGUCCGCA
ACUUCAGAUGCGGCCAGCCCCUGCAGAACAGGGUGCAGCUGAAGGGCCGGGA
CCUGCUGACCCUGAAGAACUUCACCGGCGAAGAGAUCAGGUACAUGCUGUGG
CUGAGCGCCGACCUGAAGUUCCGGAUCAAGCAGAAGGGCGAGUACCUGCCCC
UGCUGCAAGGCAAGAGCCUGGGCAUGAUCUUCGAGAAGCGGAGCACCCGGAC
CCGGCUGAGCACCGAGACAGGCUUUGCCCUGCUGGGAGGCCACCCCUGCUUU
CUGACCACCCAGGACAUCCACCUGGGCGUGAACGAGAGCCUGACCGACACCG
CCAGAGUGCUGAGCAGCAUGGCCGACGCCGUGCUGGCCCGGGUGUACAAGCA
GAGCGACCUGGACACCCUGGCCAAAGAGGCCAGCAUCCCCAUCAUCAACGGC
CUGAGCGACCUGUACCACCCCAUCCAGAUCCUGGCCGACUACCUGACCCUGCA
GGAACACUACAGCUCCCUGAAGGGCCUGACCCUGAGCUGGAUCGGCGACGGC
AACAACAUCCUGCACAGCAUCAUGAUGAGCGCCGCCAAGUUCGGCAUGCAUC
UGCAGGCCGCCACCCCCAAGGGCUACGAGCCUGAUGCCAGCGUGACCAAGCU
GGCCGAGCAGUACGCCAAAGAGAACGGCACCAAGCUGCUGCUGACCAACGAC
CCCCUGGAAGCCGCCCACGGCGGCAACGUGCUGAUCACCGACACCUGGAUCA
GCAUGGGCCAGGAAGAGGAAAAGAAGAAGCGGCUGCAGGCCUUCCAGGGCUA
CCAGGUCACAAUGAAGACCGCCAAGGUGGCCGCCAGCGACUGGACCUUCCUG
CACUGCCUGCCCCGGAAGCCCGAAGAGGUGGACGACGAGGUGUUCUACAGCC
CCCGGUCCCUGGUGUUCCCCGAGGCCGAGAACCGGAAGUGGACCAUUAUGGC
CGUGAUGGUGUCCCUGCUGACCGACUACUCCCCCCAGCUGCAGAAGCCCAAG
UUCUAGAUAAGUGAACUCGAGCUAGUGACUGACUAGGAUCUGGUUACCACUA
AACCAGCCUCAAGAACACCCGAAUGGAGUCUCUAAGCUACAUAAUACCAACU
UACACUUACAAAAUGUUGUCCCCCAAAAUGUAGCCAUUCGUAUCUGCUCCUA
AUAAAAAGAAAGUUUCUUCACAUUCUAG
>mARM1806
(SEQ ID NO: 80)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUGUUCAACCUGAGGAUC
CUGCUGAACAACGCAGCUUUCAGGAACGGCCACAACUUCAUGGUGAGGAACU
UCCGGUGCGGCCAGCCCCUGCAGAACAAGGUGCAGCUGAAGGGCAGGGACCU
GCUGACCCUGAAGAACUUCACCGGAGAGGAGAUCAAGUACAUGCUGUGGCUG
AGCGCAGACCUGAAGUUCAGGAUCAAGCAGAAGGGAGAGUACCUGCCCCUGC
UGCAGGGGAAGUCCCUGGGCAUGAUCUUCGAGAAGAGGAGUACCAGGACCAG
GCUGAGCACCGAAACCGGCUUCGCCCUGCUGGGAGGACACCCCUGCUUCCUG
ACCACCCAGGACAUCCACCUGGGCGUGAACGAGAGUCUGACCGACACCGCCA
GGGUGCUGUCUAGCAUGGCCGACGCCGUGCUGGCCAGGGUGUACAAGCAGUC
AGACCUGGACACCCUGGCUAAGGAGGCCAGCAUCCCCAUCAUCAACGGCCUG
AGCGACCUGUACCACCCCAUCCAGAUCCUGGCUGACUACCUGACCCUGCAGG
AGCACUACAGCUCUCUGAAGGGCCUGACCCUGAGCUGGAUCGGCGACGGGAA
CAACAUCCUGCACAGCAUCAUGAUGAGCGCCGCCAAGUUCGGCAUGCACCUG
CAGGCCGCUACCCCCAAGGGUUACGAGCCCGACGCCAGCGUGACCAAGCUGG
CAGAGCAGUACGCCAAGGAGAACGGCACCAAGCUGCUGCUGACCAACGACCC
CCUGGAGGCCGCCCACGGAGGCAACGUGCUGAUCACCGACACCUGGAUCAGC
AUGGGACAGGAGGAGGAGAAGAAGAAGCGGCUGCAGGCUUUCCAGGGUUAC
CAGGUGACCAUGAAGACCGCCAAGGUGGCUGCCAGCGACUGGACCUUCCUGC
ACUGCCUGCCCAGGAAGCCCGAGGAGGUGGACGACGAGGUGUUCUACUCUCC
CAGGAGCCUGGUGUUCCCCGAGGCCGAGAACAGGAAGUGGACCAUCAUGGCU
GUGAUGGUGUCCCUGCUGACCGACUACAGCCCCCAGCUGCAGAAGCCCAAGU
UCUGAAUAAGUGAACUCGAGCUAGUGACUGACUAGGAUCUGGUUACCACUAA
ACCAGCCUCAAGAACACCCGAAUGGAGUCUCUAAGCUACAUAAUACCAACUU
ACACUUACAAAAUGUUGUCCCCCAAAAUGUAGCCAUUCGUAUCUGCUCCUAA
UAAAAAGAAAGUUUCUUCACAUUCUAG
>mARM1808
(SEQ ID NO: 81)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUGUUCAACCUGAGGAUC
CUGCUGAACAACGCAGCUUUCAGGAACGGCCACAACUUCAUGGUGAGGAACU
UCCGGUGCGGCCAGCCCCUGCAGAACAAGGUGCAGCUGAAGGGCAGGGACCU
GCUGACCCUGAAGAACUUCACCGGAGAGGAGAUCAAGUACAUGCUGUGGCUG
AGCGCAGACCUGAAGUUCAGGAUCAAGCAGAAGGGAGAGUACCUGCCCCUGC
UGCAGGGGAAGUCCCUGGGCAUGAUCUUCGAGAAGAGGAGUACCAGGACCAG
GCUGAGCACCGAAACCGGCUUCGCCCUGCUGGGAGGACACCCCUGCUUCCUG
ACGACCCAGGACAUCCACCUCGGAGUGAACGAAUCCCUCACCGAUACCGCCC
GGGUGUUAUCAAGCAUGGCAGAUGCCGUGCUGGCCAGGGUGUACAAACAGUC
CGAUCUGGACACUCUGGCUAAGGAGGCCAGCAUCCCCAUCAUCAACGGCCUG
AGCGACCUGUACCACCCCAUCCAGAUCCUGGCUGACUACCUGACCCUGCAGG
AGCACUACAGCUCUCUGAAGGGCCUGACCCUGAGCUGGAUCGGCGACGGGAA
CAACAUCCUGCACUCCAUCAUGAUGUCCGCCGCGAAGUUCGGAAUGCAUCUG
CAAGCCGCCACGCCAAAAGGAUACGAACCGGAUGCGCCCGUGACAAAGUUGG
CGGAACAGUACGCUAAGGAGAACGGAACCAAGCUGCUGCUGACCAACGACCC
CCUGGAGGCCGCCCACGGAGGCAACGUGCUGAUCACCGACACCUGGAUCAGC
AUGGGACAGGAGGAGGAGAAGAAGAAGCGGCUGCAGGCUUUCCAGGGUUAC
CAGGUGACCAUGAAGACCGCCAAGGUGGCUGCCAGCGACUGGACCUUCCUGC
ACUGCCUGCCCAGGAAGCCCGAGGAGGUGGACGACGAGGUGUUCUACUCUCC
CAGGAGCCUGGUGUUCCCCGAGGCCGAGAACAGGAAGUGGACCAUCAUGGCU
GUGAUGGUGUCCCUGCUGACCGACUACAGCCCCCAGCUGCAGAAGCCCAAGU
UCUGAAUAAGUGAACUCGAGCUAGUGACUGACUAGGAUCUGGUUACCACUAA
ACCAGCCUCAAGAACACCCGAAUGGAGUCUCUAAGCUACAUAAUACCAACUU
ACACUUACAAAAUGUUGUCCCCCAAAAUGUAGCCAUUCGUAUCUGCUCCUAA
UAAAAAGAAAGUUUCUUCACAUUCUAG
>mARM1809
(SEQ ID NO: 82)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUGUUCAACCUGCGCAUC
CUGCUGAACAACGCCGCCUUCCGCAACGGCCACAACUUCAUGGUGCGCAACU
UCCGCUGCGGCCAGCCCCUGCAGAACAAGGUGCAGCUGAAGGGCCGCGACCU
GCUGACCCUGAAGAACUUCACCGGCGAGGAGAUCAAGUACAUGCUGUGGCUG
AGCGCCGACCUGAAGUUCCGCAUCAAGCAGAAGGGCGAGUACCUGCCCCUGC
UGCAGGGCAAGAGCCUGGGCAUGAUCUUCGAGAAGCGCAGCACCCGCACCCG
CCUGAGCACCGAGACAGGCUUCGCCCUGCUGGGCGGCCACCCCUGCUUCCUG
ACCACCCAGGACAUCCACCUGGGCGUGAACGAGAGCCUGACCGACACCGCCC
GCGUGCUGAGCAGCAUGGCCGACGCCGUGCUGGCCCGCGUGUACAAGCAGAG
CGACCUGGACACCCUGGCCAAGGAGGCCAGCAUCCCCAUCAUCAACGGCCUG
AGCGACCUGUACCACCCCAUCCAGAUCCUGGCCGACUACCUGACCCUGCAGG
AGCACUACAGCAGCCUGAAGGGCCUGACCCUGAGCUGGAUCGGCGACGGCAA
CAACAUCCUGCACAGCAUCAUGAUGAGCGCCGCCAAGUUCGGCAUGCACCUG
CAGGCCGCCACCCCCAAGGGCUACGAGCCCGACGCCAGCGUGACCAAGCUGGC
CGAGCAGUACGCCAAGGAGAACGGCACCAAGCUGCUGCUGACCAACGACCCC
CUGGAGGCCGCCCACGGCGGCAACGUGCUGAUCACCGACACCUGGAUCAGCA
UGGGCCAGGAGGAGGAGAAGAAGAAGCGCCUGCAGGCCUUCCAGGGCUACCA
GGUGACCAUGAAGACCGCCAAGGUGGCCGCCAGCGACUGGACCUUCCUGCAC
UGCCUGCCCCGCAAGCCCGAGGAGGUGGACGACGAGGUGUUCUACAGCCCCC
GCAGCCUGGUGUUCCCCGAGGCCGAGAACCGCAAGUGGACCAUCAUGGCCGU
GAUGGUGAGCCUGCUGACCGACUACAGCCCCCAGCUGCAGAAGCCCAAGUUC
UGAAUAAGUGAACUCGAGCUAGUGACUGACUAGGAUCUGGUUACCACUAAAC
CAGCCUCAAGAACACCCGAAUGGAGUCUCUAAGCUACAUAAUACCAACUUAC
ACUUACAAAAUGUUGUCCCCCAAAAUGUAGCCAUUCGUAUCUGCUCCUAAUA
AAAAGAAAGUUUCUUCACAUUCUAGAAAAAAAAAAAAAAAAAAAAAAAAAA
AAAAAAAAAAAAAAAAAAAAAAAAAAAAAAAAAAAAAAAAAAAAAAAAAAA
AAAAAAAAAAAAAAAAAAAAAAA
>mARM1816
(SEQ ID NO: 83)
AGGAAACUUAAGUCAACACAACAUAUACAAAACAAACGAAUCUCAAGCAAUC
AAGCAUUCUACUUCUAUUGCAGCAAUUUAAAUCAUUUCUUUUAAAGCAAAA
GCAAUUUUCUGAAAAUUUUCACCAUUUACGAACGAUAGCCAUGCUGUUCAAC
CUGCGCAUCCUGCUGAACAACGCCGCCUUCCGCAACGGCCACAACUUCAUGG
UGCGCAACUUCCGCUGCGGCCAGCCCCUGCAGAACAAGGUGCAGCUGAAGGG
CCGCGACCUGCUGACCCUGAAGAACUUCACCGGCGAGGAGAUCAAGUACAUG
CUGUGGCUGAGCGCCGACCUGAAGUUCCGCAUCAAGCAGAAGGGCGAGUACC
UGCCCCUGCUGCAGGGCAAGAGCCUGGGCAUGAUCUUCGAGAAGCGCAGCAC
CCGCACCCGCCUGAGCACCGAGACAGGCUUCGCCCUGCUGGGCGGCCACCCCU
GCUUCCUGACCACCCAGGACAUCCACCUGGGCGUGAACGAGAGCCUGACCGA
CACCGCCCGCGUGCUGAGCAGCAUGGCCGACGCCGUGCUGGCCCGCGUGUAC
AAGCAGAGCGACCUGGACACCCUGGCCAAGGAGGCCAGCAUCCCCAUCAUCA
ACGGCCUGAGCGACCUGUACCACCCCAUCCAGAUCCUGGCCGACUACCUGACC
CUGCAGGAGCACUACAGCAGCCUGAAGGGCCUGACCCUGAGCUGGAUCGGCG
ACGGCAACAACAUCCUGCACAGCAUCAUGAUGAGCGCCGCCAAGUUCGGCAU
GCACCUGCAGGCCGCCACCCCCAAGGGCUACGAGCCCGACGCCAGCGUGACCA
AGCUGGCCGAGCAGUACGCCAAGGAGAACGGCACCAAGCUGCUGCUGACCAA
CGACCCCCUGGAGGCCGCCCACGGCGGCAACGUGCUGAUCACCGACACCUGG
AUCAGCAUGGGCCAGGAGGAGGAGAAGAAGAAGCGCCUGCAGGCCUUCCAGG
GCUACCAGGUGACCAUGAAGACCGCCAAGGUGGCCGCCAGCGACUGGACCUU
CCUGCACUGCCUGCCCCGCAAGCCCGAGGAGGUGGACGACGAGGUGUUCUAC
AGCCCCCGCAGCCUGGUGUUCCCCGAGGCCGAGAACCGCAAGUGGACCAUCA
UGGCCGUGAUGGUGAGCCUGCUGACCGACUACAGCCCCCAGCUGCAGAAGCC
CAAGUUCUGACUAGUGACUGACUAGGAUCUGGUUACCACUAAACCAGCCUCA
AGAACACCCGAAUGGAGUCUCUAAGCUACAUAAUACCAACUUACACUUACAA
AAUGUUGUCCCCCAAAAUGUAGCCAUUCGUAUCUGCUCCUAAUAAAAAGAAA
GUUUCUUCACAUUCUAG
>mARM1822
(SEQ ID NO: 84)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAACUUGAGAAUC
CUGCUGAACAACGCCGCCUUUCGCAACGGUCACAAUUUUAUGGUCAGAAACU
UCAGAUGCGGACAGCCCCUCCAAAACAAGGUCCAGCUGAAGGGCCGCGAUCU
CCUCACCCUGAAGAACUUCACGGGGGAGGAGAUCAAGUACAUGCUGUGGCUC
UCCGCUGACCUGAAGUUCAGGAUCAAGCAGAAGGGAGAAUAUCUGCCGCUGC
UGCAAGGGAAGUCCCUGGGGAUGAUUUUCGAGAAGCGGAGCACCCGGACUCG
GCUCUCCACUGAAACUGGUUUCGCCCUUCUGGGCGGUCACCCCUGCUUCCUG
ACCACUCAAGACAUUCACCUCGGAGUGAACGAGUCCUUGACUGACACCGCCC
GGGUGCUGUCGAGCAUGGCAGACGCCGUGCUAGCCCGCGUGUACAAGCAGUC
AGACCUCGAUACCCUGGCCAAGGAGGCUUCGAUCCCGAUCAUCAACGGGUUG
UCCGACCUGUACCACCCGAUUCAGAUUCUCGCCGACUACCUCACCCUGCAAG
AGCAUUACAGCUCCCUGAAGGGGCUUACCCUGUCCUGGAUUGGCGACGGAAA
CAACAUCCUGCACUCCAUUAUGAUGUCGGCGGCCAAGUUCGGCAUGCACCUC
CAAGCCGCGACCCCUAAGGGUUACGAACCAGACGCGUCAGUGACUAAGCUGG
CCGAACAGUACGCAAAGGAAAAUGGCACGAAGCUGCUCCUGACCAACGAUCC
GUUGGAAGCCGCCCAUGGCGGAAAUGUGCUCAUCACCGACACCUGGAUCUCG
AUGGGACAGGAGGAAGAGAAGAAGAAGCGGCUGCAGGCGUUCCAGGGCUACC
AGGUCACCAUGAAAACUGCCAAGGUGGCCGCCAGCGACUGGACCUUCCUGCA
CUGCCUUCCGCGCAAGCCUGAGGAGGUGGACGAUGAAGUGUUCUACUCUCCA
CGGUCCCUGGUGUUCCCCGAGGCGGAGAACCGCAAAUGGACCAUCAUGGCUG
UGAUGGUCAGCCUGCUGACCGAUUACAGCCCUCAGUUGCAAAAGCCGAAGUU
UUGAUAACUCGAGCUAGUGACUGACUAGGAUCUGGUUACCACUAAACCAGCC
UCAAGAACACCCGAAUGGAGUCUCUAAGCUACAUAAUACCAACUUACACUUA
CAAAAUGUUGUCCCCCAAAAUGUAGCCAUUCGUAUCUGCUCCUAAUAAAAAG
AAAGUUUCUUCACAUUCUAG
>mARM1823
(SEQ ID NO: 85)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUGUUCAACCUCCGCAUC
CUCCUCAACAACGCCGCAUUCAGAAACGGGCACAACUUCAUGGUCAGAAACU
UCCGCUGCGGGCAACCCCUACAAAACAAGGUCCAGCUCAAGGGGCGGGACCU
CCUGACCCUGAAGAACUUCACCGGCGAAGAGAUCAAGUACAUGCUGUGGCUC
UCCGCCGACCUGAAGUUCCGCAUCAAGCAGAAGGGAGAGUACCUCCCGCUGC
UGCAAGGGAAGUCGCUGGGGAUGAUCUUCGAGAAGCGGUCAACCAGAACCCG
GCUGUCAACCGAAACCGGGUUCGCACUGCUGGGGGGACACCCGUGCUUCCUG
ACCACCCAAGACAUCCACCUGGGAGUGAACGAAUCGCUGACCGACACCGCCC
GCGUGCUGAGCUCAAUGGCGGACGCCGUGCUGGCCCGCGUGUACAAGCAGUC
CGACCUGGACACCCUGGCCAAGGAAGCGUCCAUCCCGAUCAUCAACGGACUG
UCCGACCUGUACCACCCGAUCCAGAUCCUGGCAGACUACCUGACCCUGCAAG
AACACUACAGCUCCCUGAAGGGCCUGACCCUGUCAUGGAUCGGGGACGGGAA
CAACAUCCUGCACUCCAUAAUGAUGUCAGCCGCCAAGUUCGGAAUGCACCUC
CAAGCCGCAACCCCGAAGGGCUACGAACCGGACGCAUCAGUGACCAAACUGG
CCGAGCAGUACGCCAAGGAAAACGGCACCAAGCUCCUGCUGACCAACGACCC
GCUGGAGGCCGCACACGGGGGGAACGUGCUGAUCACCGACACCUGGAUCUCC
AUGGGACAGGAGGAGGAAAAGAAGAAGCGGCUGCAGGCGUUCCAGGGGUAC
CAGGUCACCAUGAAAACCGCGAAGGUCGCGGCAUCAGACUGGACCUUCCUGC
ACUGCCUGCCCCGGAAGCCGGAAGAGGUGGACGACGAGGUGUUCUACUCGCC
GCGCUCGCUGGUGUUCCCCGAGGCGGAGAACAGGAAGUGGACCAUCAUGGCG
GUGAUGGUCAGCCUCCUGACCGACUACUCGCCGCAGCUGCAGAAGCCGAAGU
UCUGAUAACUCGAGCUAGUGACUGACUAGGAUCUGGUUACCACUAAACCAGC
CUCAAGAACACCCGAAUGGAGUCUCUAAGCUACAUAAUACCAACUUACACUU
ACAAAAUGUUGUCCCCCAAAAUGUAGCCAUUCGUAUCUGCUCCUAAUAAAAA
GAAAGUUUCUUCACAUUCUAGAAAAAAAAAAAAAAAAAAAAAAAAAAAAAA
AAAAAAAAAAAAAAAAAAAAAAAAAAAAAAAAAAAAAAAAAAAAAAAAAAA
AAAAAAAAAAAAAAAAAAA
>mARM1840
(SEQ ID NO: 86)
CUCCCUCCCCCCCCCCUAACGUUACUGGCCGAAGCCGCUUGGAAUAAGGCCGG
UGUGCGUUUGUCUAUAUGUUAUUUUCCACCAUAUUGCCGUCUUUUGGCAAUG
UGAGGGCCCGGAAACCUGGCCCUGUCUUCUUGACGAGCAUUCCUAGGGGUCU
UUCCCCUCUCGCCAAAGGAAUGCAAGGUCUGUUGAAUGUCGUGAAGGAAGCA
GUUCCUCUGGAAGCUUCUUGAAGACAAACAACGUCUGUAGCGACCCUUUGCA
GGCAGCGGAACCCCCCACCUGGCGACAGGUGCCUCUGCGGCCAAAAGCCACG
UGUAUAAGAUACACCUGCAAAGGCGGCACAACCCCAGUGCCACGUUGUGAGU
UGGAUAGUUGUGGAAAGAGUCAAAUGGCUCUCCUCAAGCGUAUUCAACAAG
GGGCUGAAGGAUGCCCAGAAGGUACCCCAUUGUAUGGGAUCUGAUCUGGGGC
CUCGGUGCACAUGCUUUACGUGUGUUUAGUCGAGGUUAAAAAACGUCUAGGC
CCCCCGAACCACGGGGACGUGGUUUUCCUUUGAAAAACACGAUGAUAAUAUG
CUUUUCAAUCUCCGCAUCCUCCUUAACAACGCCGCGUUUAGAAACGGCCACA
ACUUCAUGGUCCGGAACUUCAGAUGUGGCCAGCCGCUUCAAAACAAGGUCCA
GCUGAAGGGCCGGGAUCUUCUGACCCUGAAGAACUUUACUGGCGAAGAGAUC
AAGUACAUGCUCUGGCUCUCCGCGGACUUGAAGUUCCGCAUUAAGCAGAAGG
GGGAAUACCUUCCGCUGCUUCAAGGAAAGAGCCUCGGCAUGAUCUUUGAGAA
GCGCUCAACCAGGACCCGCCUUUCUACUGAAACUGGGUUCGCGCUGCUCGGU
GGCCACCCCUGCUUCCUGACGACCCAGGACAUCCACCUCGGAGUGAACGAAU
CCCUCACCGAUACCGCCCGGGUGUUAUCGAGCAUGGCAGAUGCCGUGCUGGC
CAGGGUGUACAAACAGUCCGAUCUGGACACUCUGGCCAAGGAGGCGUCAAUU
CCUAUUAUCAACGGCCUUAGUGACCUCUACCAUCCGAUUCAGAUCCUGGCCG
AUUACCUCACCCUGCAAGAACACUACAGCUCCCUGAAGGGUCUGACAUUGUC
CUGGAUCGGCGACGGCAACAACAUUCUCCAUUCCAUCAUGAUGUCCGCCGCA
AAAUUCGGCAUGCAUCUUCAAGCCGCCACGCCGAAGGGUUACGAGCCCGACG
CUUCCGUGACUAAGCUCGCCGAGCAGUACGCUAAGGAGAACGGAACCAAGCU
UCUGCUGACUAACGACCCACUAGAAGCAGCCCACGGGGGCAACGUGCUUAUU
ACUGACACCUGGAUCUCCAUGGGCCAGGAAGAAGAGAAAAAGAAGCGGCUGC
AGGCGUUCCAGGGAUAUCAGGUCACCAUGAAAACCGCCAAGGUCGCUGCCUC
CGACUGGACCUUCCUGCACUGCCUGCCUCGCAAGCCUGAAGAAGUGGACGAC
GAGGUGUUCUACUCGCCACGGAGCCUCGUGUUCCCCGAGGCCGAGAAUAGAA
AGUGGACCAUCAUGGCCGUGAUGGUGUCACUGCUCACCGACUACAGCCCGCA
GCUUCAGAAGCCCAAGUUCUGAAUAAGUAGAUAGUGCAGUCACUGGCACAAC
GCGUUGCCCGGUAAGCCAAUCGGGUAUACACGGUCGUCAUACUGCAGACAGG
GUUCUUCUACUUUGCAAGAUAGUCUAGAGUAGUAAAAUAAAUAGUAUAAGU
CUAG
>mARM1841
(SEQ ID NO: 87)
CUCCCUCCCCCCCCCCUAACGUUACUGGCCGAAGCCGCUUGGAAUAAGGCCGG
UGUGCGUUUGUCUAUAUGUUAUUUUCCACCAUAUUGCCGUCUUUUGGCAAUG
UGAGGGCCCGGAAACCUGGCCCUGUCUUCUUGACGAGCAUUCCUAGGGGUCU
UUCCCCUCUCGCCAAAGGAAUGCAAGGUCUGUUGAAUGUCGUGAAGGAAGCA
GUUCCUCUGGAAGCUUCUUGAAGACAAACAACGUCUGUAGCGACCCUUUGCA
GGCAGCGGAACCCCCCACCUGGCGACAGGUGCCUCUGCGGCCAAAAGCCACG
UGUAUAAGAUACACCUGCAAAGGCGGCACAACCCCAGUGCCACGUUGUGAGU
UGGAUAGUUGUGGAAAGAGUCAAAUGGCUCUCCUCAAGCGUAUUCAACAAG
GGGCUGAAGGAUGCCCAGAAGGUACCCCAUUGUAUGGGAUCUGAUCUGGGGC
CUCGGUGCACAUGCUUUACGUGUGUUUAGUCGAGGUUAAAAAACGUCUAGGC
CCCCCGAACCACGGGGACGUGGUUUUCCUUUGAAAAACACGAUGAUAAUAUG
CUUUUCAACCUGAGAAUCCUCUUGAACAAUGCUGCUUUUCGGAAUGGCCACA
ACUUUAUGGUUCGGAACUUCCGUUGCGGCCAGCCUUUACAAAACAAGGUCCA
GCUGAAGGGCCGGGAUUUGCUCACACUAAAGAACUUUACUGGAGAAGAGAUC
AAGUACAUGCUAUGGCUGUCGGCCGACCUGAAGUUCCGUAUCAAGCAGAAGG
GAGAAUACCUUCCGCUGCUUCAAGGAAAGAGCCUCGGCAUGAUCUUUGAGAA
GCGCUCAACCAGGACCCGCCUUUCUACUGAAACUGGGUUCGCGCUGCUCGGU
GGCCACCCCUGCUUCCUGACGACCCAGGACAUCCACCUCGGAGUGAACGAAU
CCCUCACCGAUACCGCCCGGGUGUUAUCGAGCAUGGCAGAUGCCGUGCUGGC
CAGGGUGUACAAACAGUCCGAUCUCGAUACCUUGGCAAAGGAGGCUUCCAUU
CCCAUCAUCAACGGCCUGAGCGACCUGUACCACCCAAUCCAAAUCCUGGCUG
ACUACCUGACCCUGCAAGAGCACUACAGCAGCCUGAAGGGUCUGACCCUGUC
AUGGAUUGGCGAUGGAAACAAUAUUCUGCACUCCAUCAUGAUGUCCGCCGCG
AAGUUCGGAAUGCAUCUGCAAGCCGCCACUCCAAAAGGAUACGAACCGGAUG
CAUCCGUGACCAAGUUGGCGGAACAGUACGCGAAGGAGAACGGAACCAAGCU
CCUGCUGACUAACGACCCGCUCGAGGCUGCGCAUGGGGGUAACGUGCUGAUU
ACGGACACCUGGAUCUCCAUGGGGCAGGAGGAAGAGAAGAAGAAGAGACUGC
AGGCAUUCCAGGGGUACCAGGUCACCAUGAAAACCGCAAAAGUGGCAGCUUC
GGACUGGACUUUCCUGCAUUGCCUGCCGAGGAAGCCGGAGGAAGUCGACGAC
GAAGUGUUCUACUCGCCUCGGUCCCUGGUGUUCCCCGAGGCCGAAAACCGGA
AGUGGACCAUCAUGGCCGUGAUGGUGUCCUUGCUGACUGACUAUAGCCCGCA
GCUGCAGAAGCCUAAGUUCUGAAUAAGUAGAUAGUGCAGUCACUGGCACAAC
GCGUUGCCCGGUAAGCCAAUCGGGUAUACACGGUCGUCAUACUGCAGACAGG
GUUCUUCUACUUUGCAAGAUAGUCUAGAGUAGUAAAAUAAAUAGUAUAAGU
CUAG
>mARM1842
(SEQ ID NO: 88)
CUCCCUCCCCCCCCCCUAACGUUACUGGCCGAAGCCGCUUGGAAUAAGGCCGG
UGUGCGUUUGUCUAUAUGUUAUUUUCCACCAUAUUGCCGUCUUUUGGCAAUG
UGAGGGCCCGGAAACCUGGCCCUGUCUUCUUGACGAGCAUUCCUAGGGGUCU
UUCCCCUCUCGCCAAAGGAAUGCAAGGUCUGUUGAAUGUCGUGAAGGAAGCA
GUUCCUCUGGAAGCUUCUUGAAGACAAACAACGUCUGUAGCGACCCUUUGCA
GGCAGCGGAACCCCCCACCUGGCGACAGGUGCCUCUGCGGCCAAAAGCCACG
UGUAUAAGAUACACCUGCAAAGGCGGCACAACCCCAGUGCCACGUUGUGAGU
UGGAUAGUUGUGGAAAGAGUCAAAUGGCUCUCCUCAAGCGUAUUCAACAAG
GGGCUGAAGGAUGCCCAGAAGGUACCCCAUUGUAUGGGAUCUGAUCUGGGGC
CUCGGUGCACAUGCUUUACGUGUGUUUAGUCGAGGUUAAAAAACGUCUAGGC
CCCCCGAACCACGGGGACGUGGUUUUCCUUUGAAAAACACGAUGAUAAUAUG
CUGUUCAACCUGCGCAUCCUGCUGAACAACGCCGCCUUCCGCAACGGCCACA
ACUUCAUGGUGCGCAACUUCCGCUGCGGCCAGCCCCUGCAGAACAAGGUGCA
GCUGAAGGGCCGCGACCUGCUGACCCUGAAGAACUUCACCGGCGAGGAGAUC
AAGUACAUGCUGUGGCUGAGCGCCGACCUGAAGUUCCGCAUCAAGCAGAAGG
GCGAGUACCUGCCCCUGCUGCAGGGCAAGAGCCUGGGCAUGAUCUUCGAGAA
GCGCAGCACCCGCACCCGCCUGAGCACCGAGACAGGCCUGGCCCUGCUGGGCG
GCCACCCCUGCUUCCUGACCACCCAGGACAUCCACCUGGGCGUGAACGAGAG
CCUGACCGACACCGCCCGCGUGCUGAGCAGCAUGGCCGACGCCGUGCUGGCCC
GCGUGUACAAGCAGAGCGACCUGGACACCCUGGCCAAGGAGGCCAGCAUCCC
CAUCAUCAACGGCCUGAGCGACCUGUACCACCCCAUCCAGAUCCUGGCCGAC
UACCUGACCCUGCAGGAGCACUACAGCAGCCUGAAGGGCCUGACCCUGAGCU
GGAUCGGCGACGGCAACAACAUCCUGCACAGCAUCAUGAUGAGCGCCGCCAA
GUUCGGCAUGCACCUGCAGGCCGCCACCCCCAAGGGCUACGAGCCCGACGCCA
GCGUGACCAAGCUGGCCGAGCAGUACGCCAAGGAGAACGGCACCAAGCUGCU
GCUGACCAACGACCCCCUGGAGGCCGCCCACGGCGGCAACGUGCUGAUCACC
GACACCUGGAUCAGCAUGGGCCAGGAGGAGGAGAAGAAGAAGCGCCUGCAGG
CCUUCCAGGGCUACCAGGUGACCAUGAAGACCGCCAAGGUGGCCGCCAGCGA
CUGGACCUUCCUGCACUGCCUGCCCCGCAAGCCCGAGGAGGUGGACGACGAG
GUGUUCUACAGCCCCCGCAGCCUGGUGUUCCCCGAGGCCGAGAACCGCAAGU
GGACCAUCAUGGCCGUGAUGGUGAGCCUGCUGACCGACUACAGCCCCCAGCU
GCAGAAGCCCAAGUUCUGAAUAAGUAGAUAGUGCAGUCACUGGCACAACGCG
UUGCCCGGUAAGCCAAUCGGGUAUACACGGUCGUCAUACUGCAGACAGGGUU
CUUCUACUUUGCAAGAUAGUCUAGAGUAGUAAAAUAAAUAGUAUAAGUCUA
G
>mARM1843
(SEQ ID NO: 89)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAAUCUCCGCAUC
CUCCUUAACAACGCCGCGUUUAGAAACGGCCACAACUUCAUGGUCCGGAACU
UCAGAUGUGGCCAGCCGCUUCAAAACAAGGUCCAGCUGAAGGGCCGGGAUCU
UCUGACCCUGAAGAACUUUACUGGCGAAGAGAUCAAGUACAUGCUCUGGCUC
UCCGCGGACUUGAAGUUCCGCAUUAAGCAGAAGGGGGAAUACCUUCCGCUGC
UUCAAGGAAAGAGCCUCGGCAUGAUCUUUGAGAAGCGCUCAACCAGGACCCG
CCUUUCUACUGAAACUGGGUUCGCGCUGCUCGGUGGCCACCCCUGCUUCCUG
ACGACCCAGGACAUCCACCUCGGAGUGAACGAAUCCCUCACCGAUACCGCCC
GGGUGUUAUCGAGCAUGGCAGAUGCCGUGCUGGCCAGGGUGUACAAACAGUC
CGAUCUGGACACUCUGGCCAAGGAGGCGUCAAUUCCUAUUAUCAACGGCCUU
AGUGACCUCUACCAUCCGAUUCAGAUCCUGGCCGAUUACCUCACCCUGCAAG
AACACUACAGCUCCCUGAAGGGUCUGACAUUGUCCUGGAUCGGCGACGGCAA
CAACAUUCUCCAUUCCAUCAUGAUGUCCGCCGCAAAAUUCGGCAUGCAUCUU
CAAGCCGCCACGCCGAAGGGUUACGAGCCCGACGCUUCCGUGACUAAGCUCG
CCGAGCAGUACGCUAAGGAGAACGGAACCAAGCUUCUGCUGACUAACGACCC
ACUAGAAGCAGCCCACGGGGGCAACGUGCUUAUUACUGACACCUGGAUCUCC
AUGGGCCAGGAAGAAGAGAAAAAGAAGCGGCUGCAGGCGUUCCAGGGAUAU
CAGGUCACCAUGAAAACCGCCAAGGUCGCUGCCUCCGACUGGACCUUCCUGC
ACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCC
GUGAUGGUGUCACUGCUCACCGACUACAGCCCGCAGCUUCAGAAGCCCAAGU
UCUAGCUCGAGCUAGUGACUGACUAGGAUCUGGUUACCACUAAACCAGCCUC
AAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAA
AGUUUCUUCACAUUCUAG
>mARM1844
(SEQ ID NO: 90)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAAUCUCCGCAUC
CUCCUUAACAACGCCGCGUUUAGAAACGGCCACAACUUCAUGGUCCGGAACU
UCAGAUGUGGCCAGCCGCUUCAAAACAAGGUCCAGCUGAAGGGCCGGGAUCU
UCUGACCCUGAAGAACUUUACUGGCGAAGAGAUCAAGUACAUGCUCUGGCUC
UCCGCGGACUUGAAGUUCCGCAUUAAGCAGAAGGGGGAAUACCUUCCGCUGC
UUCAAGGAAAGAGCCUCGGCAUGAUCUUUGAGAAGCGCUCAACCAGGACCCG
CCUUUCUACUGAAACUGGGUUCGCGCUGCUCGGUGGCCACCCCUGCUUCCUG
ACGACCCAGGACAUCCACCUCGGAGUGAACGAAUCCCUCACCGAUACCGCCC
GGGUGUUAUCGAGCAUGGCAGAUGCCGUGCUGGCCAGGGUGUACAAACAGUC
CGAUCUGGACACUCUGGCCAAGGAGGCGUCAAUUCCUAUUAUCAACGGCCUU
AGUGACCUCUACCAUCCGAUUCAGAUCCUGGCCGAUUACCUCACCCUGCAAG
AACACUACAGCUCCCUGAAGGGUCUGACAUUGUCCUGGAUCGGCGACGGCAA
CAACAUUCUCCAUUCCAUCAUGAUGUCCGCCGCAAAAUUCGGCAUGCAUCUU
CAAGCCGCCACGCCGAAGGGUUACGAGCCCGACGCUUCCGUGACUAAGCUCG
CCGAGCAGUACGCUAAGGAGAACGGAACCAAGCUUCUGCUGACUAACGACCC
ACUAGAAGCAGCCCACGGGGGCAACGUGCUUAUUACUGACACCUGGAUCUCC
AUGGGCCAGGAAGAAGAGAAAAAGAAGCGGCUGCAGGCGUUCCAGGGAUAU
CAGGUCACCAUGAAAACCGCCAAGGUCGCUGCCUCCGACUGGACCUUCCUGC
ACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCC
GUGAUGGUGUCACUGCUCACCGACUACAGCCCGCAGCUUCAGAAGCCCAAGU
UCUAGCUCGAGCUAGUGACUGACUAGGAUCUGGUUACCACUAAACCAGCCUC
AAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAA
AGUUUCUUCACAUUCUAG
>mARM1845
(SEQ ID NO: 91)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAAUCUCCGCAUC
CUCCUUAACAACGCCGCGUUUAGAAACGGCCACAACUUCAUGGUCCGGAACU
UCAGAUGUGGCCAGCCGCUUCAAAACAAGGUCCAGCUGAAGGGCCGGGAUCU
UCUGACCCUGAAGAACUUUACUGGCGAAGAGAUCAAGUACAUGCUCUGGCUC
UCCGCGGACUUGAAGUUCCGCAUUAAGCAGAAGGGGGAAUACCUUCCGCUGC
UUCAAGGAAAGAGCCUCGGCAUGAUCUUUGAGAAGCGCUCAACCAGGACCCG
CCUUUCUACUGAAACUGGGUUCGCGCUGCUCGGUGGCCACCCCUGCUUCCUG
ACGACCCAGGACAUCCACCUCGGAGUGAACGAAUCCCUCACCGAUACCGCCC
GGGUGUUAUCGAGCAUGGCAGAUGCCGUGCUGGCCAGGGUGUACAAACAGUC
CGAUCUGGACACUCUGGCCAAGGAGGCGUCAAUUCCUAUUAUCAACGGCCUU
AGUGACCUCUACCAUCCGAUUCAGAUCCUGGCCGAUUACCUCACCCUGCAAG
AACACUACAGCUCCCUGAAGGGUCUGACAUUGUCCUGGAUCGGCGACGGCAA
CAACAUUCUCCAUUCCAUCAUGAUGUCCGCCGCAAAAUUCGGCAUGCAUCUU
CAAGCCGCCACGCCGAAGGGUUACGAGCCCGACGCUUCCGUGACUAAGCUCG
CCGAGCAGUACGCUAAGGAGAACGGAACCAAGCUUCUGCUGACUAACGACCC
ACUAGAAGCAGCCCACGGGGGCAACGUGCUUAUUACUGACACCUGGAUCUCC
AUGGGCCAGGAAGAAGAGAAAAAGAAGCGGCUGCAGGCGUUCCAGGGAUAU
CAGGUCACCAUGAAAACCGCCAAGGUCGCUGCCUCCGACUGGACCUUCCUGC
ACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCC
GUGAUGGUGUCACUGCUCACCGACUACAGCCCGCAGCUUCAGAAGCCCAAGU
UCUAGCUCGAGCUAGUGACUGACUAGGAUCUGGUUACCACUAAACCAGCCUC
AAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAA
AGUUUCUUCACAUUCUAG
>mARM1846
(SEQ ID NO: 92)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAAUCUCCGCAUC
CUCCUUAACAACGCCGCGUUUAGAAACGGCCACAACUUCAUGGUCCGGAACU
UCAGAUGUGGCCAGCCGCUUCAAAACAAGGUCCAGCUGAAGGGCCGGGAUCU
UCUGACCCUGAAGAACUUUACUGGCGAAGAGAUCAAGUACAUGCUCUGGCUC
UCCGCGGACUUGAAGUUCCGCAUUAAGCAGAAGGGGGAAUACCUUCCGCUGC
UUCAAGGAAAGAGCCUCGGCAUGAUCUUUGAGAAGCGCUCAACCAGGACCCG
CCUUUCUACUGAAACUGGGUUCGCGCUGCUCGGUGGCCACCCCUGCUUCCUG
ACGACCCAGGACAUCCACCUCGGAGUGAACGAAUCCCUCACCGAUACCGCCC
GGGUGUUAUCGAGCAUGGCAGAUGCCGUGCUGGCCAGGGUGUACAAACAGUC
CGAUCUGGACACUCUGGCCAAGGAGGCGUCAAUUCCUAUUAUCAACGGCCUU
AGUGACCUCUACCAUCCGAUUCAGAUCCUGGCCGAUUACCUCACCCUGCAAG
AACACUACAGCUCCCUGAAGGGUCUGACAUUGUCCUGGAUCGGCGACGGCAA
CAACAUUCUCCAUUCCAUCAUGAUGUCCGCCGCAAAAUUCGGCAUGCAUCUU
CAAGCCGCCACGCCGAAGGGUUACGAGCCCGACGCUUCCGUGACUAAGCUCG
CCGAGCAGUACGCUAAGGAGAACGGAACCAAGCUUCUGCUGACUAACGACCC
ACUAGAAGCAGCCCACGGGGGCAACGUGCUUAUUACUGACACCUGGAUCUCC
AUGGGCCAGGAAGAAGAGAAAAAGAAGCGGCUGCAGGCGUUCCAGGGAUAU
CAGGUCACCAUGAAAACCGCCAAGGUCGCUGCCUCCGACUGGACCUUCCUGC
ACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCC
GUGAUGGUGUCACUGCUCACCGACUACAGCCCGCAGCUUCAGAAGCCCAAGU
UCUAGCUCGAGCUAGUGACUGACUAGGAUCUGGUUACCACUAAACCAGCCUC
AAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAA
AGUUUCUUCACAUUCUAG
>mARM1847
(SEQ ID NO: 93)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUUUCAAUCUCCGCAUC
CUCCUUAACAACGCCGCGUUUAGAAACGGCCACAACUUCAUGGUCCGGAACU
UCAGAUGUGGCCAGCCGCUUCAAAACAAGGUCCAGCUGAAGGGCCGGGAUCU
UCUGACCCUGAAGAACUUUACUGGCGAAGAGAUCAAGUACAUGCUCUGGCUC
UCCGCGGACUUGAAGUUCCGCAUUAAGCAGAAGGGGGAAUACCUUCCGCUGC
UUCAAGGAAAGAGCCUCGGCAUGAUCUUUGAGAAGCGCUCAACCAGGACCCG
CCUUUCUACUGAAACUGGGUUCGCGCUGCUCGGUGGCCACCCCUGCUUCCUG
ACGACCCAGGACAUCCACCUCGGAGUGAACGAAUCCCUCACCGAUACCGCCC
GGGUGUUAUCGAGCAUGGCAGAUGCCGUGCUGGCCAGGGUGUACAAACAGUC
CGAUCUGGACACUCUGGCCAAGGAGGCGUCAAUUCCUAUUAUCAACGGCCUU
AGUGACCUCUACCAUCCGAUUCAGAUCCUGGCCGAUUACCUCACCCUGCAAG
AACACUACAGCUCCCUGAAGGGUCUGACAUUGUCCUGGAUCGGCGACGGCAA
CAACAUUCUCCAUUCCAUCAUGAUGUCCGCCGCAAAAUUCGGCAUGCAUCUU
CAAGCCGCCACGCCGAAGGGUUACGAGCCCGACGCUUCCGUGACUAAGCUCG
CCGAGCAGUACGCUAAGGAGAACGGAACCAAGCUUCUGCUGACUAACGACCC
ACUAGAAGCAGCCCACGGGGGCAACGUGCUUAUUACUGACACCUGGAUCUCC
AUGGGCCAGGAAGAAGAGAAAAAGAAGCGGCUGCAGGCGUUCCAGGGAUAU
CAGGUCACCAUGAAAACCGCCAAGGUCGCUGCCUCCGACUGGACCUUCCUGC
ACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCC
GUGAUGGUGUCACUGCUCACCGACUACAGCCCGCAGCUUCAGAAGCCCAAGU
UCUAGCUCGAGCUAGUGACUGACUAGGAUCUGGUUACCACUAAACCAGCCUC
AAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAA
AGUUUCUUCACAUUCUAG
>mARM1882
(SEQ ID NO: 94)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGGUGUUCAACCUCCGCAUC
CUCCUCAACAACGCCGCAUUCAGAAACGGGCACAACUUCAUGGUCAGAAACU
UCCGCUGCGGGCAACCCCUACAAAACAAGGUCCAGCUCAAGGGGCGGGACCU
CCUGACCCUGAAGAACUUCACCGGCGAAGAGAUCAAGUACAUGCUGUGGCUC
UCCGCCGACCUGAAGUUCCGCAUCAAGCAGAAGGGAGAGUACCUCCCGCUGC
UGCAAGGGAAGUCGCUGGGGAUGAUCUUCGAGAAGCGGUCAACCAGAACCCG
GCUGUCAACCGAAACCGGGUUCGCACUGCUGGGGGGACACCCGUGCUUCCUG
ACCACCCAAGACAUCCACCUGGGAGUGAACGAAUCGCUGACCGACACCGCCC
GCGUGCUGAGCUCAAUGGCGGACGCCGUGCUGGCCCGCGUGUACAAGCAGUC
CGACCUGGACACCCUGGCCAAGGAAGCGUCCAUCCCGAUCAUCAACGGACUG
UCCGACCUGUACCACCCGAUCCAGAUCCUGGCAGACUACCUGACCCUGCAAG
AACACUACAGCUCCCUGAAGGGCCUGACCCUGUCAUGGAUCGGGGACGGGAA
CAACAUCCUGCACUCCAUAAUGAUGUCAGCCGCCAAGUUCGGAAUGCACCUC
CAAGCCGCAACCCCGAAGGGCUACGAACCGGACGCAUCAGUGACCAAACUGG
CCGAGCAGUACGCCAAGGAAAACGGCACCAAGCUCCUGCUGACCAACGACCC
GCUGGAGGCCGCACACGGGGGGAACGUGCUGAUCACCGACACCUGGAUCUCC
AUGGGACAGGAGGAGGAAAAGAAGAAGCGGCUGCAGGCGUUCCAGGGGUAC
CAGGUCACCAUGAAAACCGCGAAGGUCGCGGCAUCAGACUGGACCUUCCUGC
ACUGCCUGCCCCGGAAGCCGGAAGAGGUGGACGACGAGGUGUUCUACUCGCC
GCGCUCGCUGGUGUUCCCCGAGGCGGAGAACAGGAAGUGGACCAUCAUGGCG
GUGAUGGUCAGCCUCCUGACCGACUACUCGCCGCAGCUGCAGAAGCCGAAGU
UCUGAUAACUCGAGCUAGUGACUGACUAGGAUCUGGUUACCACUAAACCAGC
CUCAAGAACACCCGAAUGGAGUCUCUAAGCUACAUAAUACCAACUUACACUU
ACAAAAUGUUGUCCCCCAAAAUGUAGCCAUUCGUAUCUGCUCCUAAUAAAAA
GAAAGUUUCUUCACAUUCUAG
>mARM1883
(SEQ ID NO: 95)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGGUGUUCAACCUCCGCAUC
CUCCUCAACAACGCCGCAUUCAGAAACGGGCACAACUUCAUGGUCAGAAACU
UCCGCUGCGGGCAACCCCUACAAAACCGGGUCCAGCUCAAGGGGCGGGACCU
CCUGACCCUGAAGAACUUCACCGGCGAAGAGAUCAAGUACAUGCUGUGGCUC
UCCGCCGACCUGAAGUUCCGCAUCAAGCAGAAGGGAGAGUACCUCCCGCUGC
UGCAAGGGAAGUCGCUGGGGAUGAUCUUCGAGAAGCGGUCAACCAGAACCCG
GCUGUCAACCGAAACCGGGUUCGCACUGCUGGGGGGACACCCGUGCUUCCUG
ACCACCCAAGACAUCCACCUGGGAGUGAACGAAUCGCUGACCGACACCGCCC
GCGUGCUGAGCUCAAUGGCGGACGCCGUGCUGGCCCGCGUGUACAAGCAGUC
CGACCUGGACACCCUGGCCAAGGAAGCGUCCAUCCCGAUCAUCAACGGACUG
UCCGACCUGUACCACCCGAUCCAGAUCCUGGCAGACUACCUGACCCUGCAAG
AACACUACAGCUCCCUGAAGGGCCUGACCCUGUCAUGGAUCGGGGACGGGAA
CAACAUCCUGCACUCCAUAAUGAUGUCAGCCGCCAAGUUCGGAAUGCACCUC
CAAGCCGCAACCCCGAAGGGCUACGAACCGGACGCAUCAGUGACCAAACUGG
CCGAGCAGUACGCCAAGGAAAACGGCACCAAGCUCCUGCUGACCAACGACCC
GCUGGAGGCCGCACACGGGGGGAACGUGCUGAUCACCGACACCUGGAUCUCC
AUGGGACAGGAGGAGGAAAAGAAGAAGCGGCUGCAGGCGUUCCAGGGGUAC
CAGGUCACCAUGAAAACCGCGAAGGUCGCGGCAUCAGACUGGACCUUCCUGC
ACUGCCUGCCCCGGAAGCCGGAAGAGGUGGACGACGAGGUGUUCUACUCGCC
GCGCUCGCUGGUGUUCCCCGAGGCGGAGAACAGGAAGUGGACCAUCAUGGCG
GUGAUGGUCAGCCUCCUGACCGACUACUCGCCGCAGCUGCAGAAGCCGAAGU
UCUGAUAACUCGAGCUAGUGACUGACUAGGAUCUGGUUACCACUAAACCAGC
CUCAAGAACACCCGAAUGGAGUCUCUAAGCUACAUAAUACCAACUUACACUU
ACAAAAUGUUGUCCCCCAAAAUGUAGCCAUUCGUAUCUGCUCCUAAUAAAAA
GAAAGUUUCUUCACAUUCUAG
>mARM1884
(SEQ ID NO: 96)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGGUGUUCAACCUCCGCAUC
CUCCUCAACAACGCCGCAUUCAGAAACGGGCACAACUUCAUGGUCAGAAACU
UCCGCUGCGGGCAACCCCUACAAAACCGGGUCCAGCUCAAGGGGCGGGACCU
CCUGACCCUGAAGAACUUCACCGGCGAAGAGAUCCGGUACAUGCUGUGGCUC
UCCGCCGACCUGAAGUUCCGCAUCAAGCAGAAGGGAGAGUACCUCCCGCUGC
UGCAAGGGAAGUCGCUGGGGAUGAUCUUCGAGAAGCGGUCAACCAGAACCCG
GCUGUCAACCGAAACCGGGUUCGCACUGCUGGGGGGACACCCGUGCUUCCUG
ACCACCCAAGACAUCCACCUGGGAGUGAACGAAUCGCUGACCGACACCGCCC
GCGUGCUGAGCUCAAUGGCGGACGCCGUGCUGGCCCGCGUGUACAAGCAGUC
CGACCUGGACACCCUGGCCAAGGAAGCGUCCAUCCCGAUCAUCAACGGACUG
UCCGACCUGUACCACCCGAUCCAGAUCCUGGCAGACUACCUGACCCUGCAAG
AACACUACAGCUCCCUGAAGGGCCUGACCCUGUCAUGGAUCGGGGACGGGAA
CAACAUCCUGCACUCCAUAAUGAUGUCAGCCGCCAAGUUCGGAAUGCACCUC
CAAGCCGCAACCCCGAAGGGCUACGAACCGGACGCAUCAGUGACCAAACUGG
CCGAGCAGUACGCCAAGGAAAACGGCACCAAGCUCCUGCUGACCAACGACCC
GCUGGAGGCCGCACACGGGGGGAACGUGCUGAUCACCGACACCUGGAUCUCC
AUGGGACAGGAGGAGGAAAAGAAGAAGCGGCUGCAGGCGUUCCAGGGGUAC
CAGGUCACCAUGAAAACCGCGAAGGUCGCGGCAUCAGACUGGACCUUCCUGC
ACUGCCUGCCCCGGAAGCCGGAAGAGGUGGACGACGAGGUGUUCUACUCGCC
GCGCUCGCUGGUGUUCCCCGAGGCGGAGAACAGGAAGUGGACCAUCAUGGCG
GUGAUGGUCAGCCUCCUGACCGACUACUCGCCGCAGCUGCAGAAGCCGAAGU
UCUGAUAACUCGAGCUAGUGACUGACUAGGAUCUGGUUACCACUAAACCAGC
CUCAAGAACACCCGAAUGGAGUCUCUAAGCUACAUAAUACCAACUUACACUU
ACAAAAUGUUGUCCCCCAAAAUGUAGCCAUUCGUAUCUGCUCCUAAUAAAAA
GAAAGUUUCUUCACAUUCUAG
>mARM1885
(SEQ ID NO: 97)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUGGUCAACCUCCGCAUC
CUCCUCAACAACGCCGCAUUCAGAAACGGGCACAACUUCAUGGUCAGAAACU
UCCGCUGCGGGCAACCCCUACAAAACAAGGUCCAGCUCAAGGGGCGGGACCU
CCUGACCCUGAAGAACUUCACCGGCGAAGAGAUCAAGUACAUGCUGUGGCUC
UCCGCCGACCUGAAGUUCCGCAUCAAGCAGAAGGGAGAGUACCUCCCGCUGC
UGCAAGGGAAGUCGCUGGGGAUGAUCUUCGAGAAGCGGUCAACCAGAACCCG
GCUGUCAACCGAAACCGGGUUCGCACUGCUGGGGGGACACCCGUGCUUCCUG
ACCACCCAAGACAUCCACCUGGGAGUGAACGAAUCGCUGACCGACACCGCCC
GCGUGCUGAGCUCAAUGGCGGACGCCGUGCUGGCCCGCGUGUACAAGCAGUC
CGACCUGGACACCCUGGCCAAGGAAGCGUCCAUCCCGAUCAUCAACGGACUG
UCCGACCUGUACCACCCGAUCCAGAUCCUGGCAGACUACCUGACCCUGCAAG
AACACUACAGCUCCCUGAAGGGCCUGACCCUGUCAUGGAUCGGGGACGGGAA
CAACAUCCUGCACUCCAUAAUGAUGUCAGCCGCCAAGUUCGGAAUGCACCUC
CAAGCCGCAACCCCGAAGGGCUACGAACCGGACGCAUCAGUGACCAAACUGG
CCGAGCAGUACGCCAAGGAAAACGGCACCAAGCUCCUGCUGACCAACGACCC
GCUGGAGGCCGCACACGGGGGGAACGUGCUGAUCACCGACACCUGGAUCUCC
AUGGGACAGGAGGAGGAAAAGAAGAAGCGGCUGCAGGCGUUCCAGGGGUAC
CAGGUCACCAUGAAAACCGCGAAGGUCGCGGCAUCAGACUGGACCUUCCUGC
ACUGCCUGCCCCGGAAGCCGGAAGAGGUGGACGACGAGGUGUUCUACUCGCC
GCGCUCGCUGGUGUUCCCCGAGGCGGAGAACAGGAAGUGGACCAUCAUGGCG
GUGAUGGUCAGCCUCCUGACCGACUACUCGCCGCAGCUGCAGAAGCCGAAGU
UCUGAUAACUCGAGCUAGUGACUGACUAGGAUCUGGUUACCACUAAACCAGC
CUCAAGAACACCCGAAUGGAGUCUCUAAGCUACAUAAUACCAACUUACACUU
ACAAAAUGUUGUCCCCCAAAAUGUAGCCAUUCGUAUCUGCUCCUAAUAAAAA
GAAAGUUUCUUCACAUUCUAG
>mARM1886
(SEQ ID NO: 98)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUGGUCAACCUCCGCAUC
CUCCUCAACAACGCCGCAUUCAGAAACGGGCACAACUUCAUGGUCAGAAACU
UCCGCUGCGGGCAACCCCUACAAAACCGGGUCCAGCUCAAGGGGCGGGACCU
CCUGACCCUGAAGAACUUCACCGGCGAAGAGAUCAAGUACAUGCUGUGGCUC
UCCGCCGACCUGAAGUUCCGCAUCAAGCAGAAGGGAGAGUACCUCCCGCUGC
UGCAAGGGAAGUCGCUGGGGAUGAUCUUCGAGAAGCGGUCAACCAGAACCCG
GCUGUCAACCGAAACCGGGUUCGCACUGCUGGGGGGACACCCGUGCUUCCUG
ACCACCCAAGACAUCCACCUGGGAGUGAACGAAUCGCUGACCGACACCGCCC
GCGUGCUGAGCUCAAUGGCGGACGCCGUGCUGGCCCGCGUGUACAAGCAGUC
CGACCUGGACACCCUGGCCAAGGAAGCGUCCAUCCCGAUCAUCAACGGACUG
UCCGACCUGUACCACCCGAUCCAGAUCCUGGCAGACUACCUGACCCUGCAAG
AACACUACAGCUCCCUGAAGGGCCUGACCCUGUCAUGGAUCGGGGACGGGAA
CAACAUCCUGCACUCCAUAAUGAUGUCAGCCGCCAAGUUCGGAAUGCACCUC
CAAGCCGCAACCCCGAAGGGCUACGAACCGGACGCAUCAGUGACCAAACUGG
CCGAGCAGUACGCCAAGGAAAACGGCACCAAGCUCCUGCUGACCAACGACCC
GCUGGAGGCCGCACACGGGGGGAACGUGCUGAUCACCGACACCUGGAUCUCC
AUGGGACAGGAGGAGGAAAAGAAGAAGCGGCUGCAGGCGUUCCAGGGGUAC
CAGGUCACCAUGAAAACCGCGAAGGUCGCGGCAUCAGACUGGACCUUCCUGC
ACUGCCUGCCCCGGAAGCCGGAAGAGGUGGACGACGAGGUGUUCUACUCGCC
GCGCUCGCUGGUGUUCCCCGAGGCGGAGAACAGGAAGUGGACCAUCAUGGCG
GUGAUGGUCAGCCUCCUGACCGACUACUCGCCGCAGCUGCAGAAGCCGAAGU
UCUGAUAACUCGAGCUAGUGACUGACUAGGAUCUGGUUACCACUAAACCAGC
CUCAAGAACACCCGAAUGGAGUCUCUAAGCUACAUAAUACCAACUUACACUU
ACAAAAUGUUGUCCCCCAAAAUGUAGCCAUUCGUAUCUGCUCCUAAUAAAAA
GAAAGUUUCUUCACAUUCUAGAAAAAAAAAAAAAAAAAAAAAAAAAAAAAA
AAAAAAAAAAAAAAAAAAAAAAAAAAAAAAAAAAAAAAAAAAAAAAAAAAA
AAAAAAAAAAAAAAAAAAA
>mARM1887
(SEQ ID NO: 99)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUGGUCAACCUCCGCAUC
CUCCUCAACAACGCCGCAUUCAGAAACGGGCACAACUUCAUGGUCAGAAACU
UCCGCUGCGGGCAACCCCUACAAAACCGGGUCCAGCUCAAGGGGCGGGACCU
CCUGACCCUGAAGAACUUCACCGGCGAAGAGAUCCGGUACAUGCUGUGGCUC
UCCGCCGACCUGAAGUUCCGCAUCAAGCAGAAGGGAGAGUACCUCCCGCUGC
UGCAAGGGAAGUCGCUGGGGAUGAUCUUCGAGAAGCGGUCAACCAGAACCCG
GCUGUCAACCGAAACCGGGUUCGCACUGCUGGGGGGACACCCGUGCUUCCUG
ACCACCCAAGACAUCCACCUGGGAGUGAACGAAUCGCUGACCGACACCGCCC
GCGUGCUGAGCUCAAUGGCGGACGCCGUGCUGGCCCGCGUGUACAAGCAGUC
CGACCUGGACACCCUGGCCAAGGAAGCGUCCAUCCCGAUCAUCAACGGACUG
UCCGACCUGUACCACCCGAUCCAGAUCCUGGCAGACUACCUGACCCUGCAAG
AACACUACAGCUCCCUGAAGGGCCUGACCCUGUCAUGGAUCGGGGACGGGAA
CAACAUCCUGCACUCCAUAAUGAUGUCAGCCGCCAAGUUCGGAAUGCACCUC
CAAGCCGCAACCCCGAAGGGCUACGAACCGGACGCAUCAGUGACCAAACUGG
CCGAGCAGUACGCCAAGGAAAACGGCACCAAGCUCCUGCUGACCAACGACCC
GCUGGAGGCCGCACACGGGGGGAACGUGCUGAUCACCGACACCUGGAUCUCC
AUGGGACAGGAGGAGGAAAAGAAGAAGCGGCUGCAGGCGUUCCAGGGGUAC
CAGGUCACCAUGAAAACCGCGAAGGUCGCGGCAUCAGACUGGACCUUCCUGC
ACUGCCUGCCCCGGAAGCCGGAAGAGGUGGACGACGAGGUGUUCUACUCGCC
GCGCUCGCUGGUGUUCCCCGAGGCGGAGAACAGGAAGUGGACCAUCAUGGCG
GUGAUGGUCAGCCUCCUGACCGACUACUCGCCGCAGCUGCAGAAGCCGAAGU
UCUGAUAACUCGAGCUAGUGACUGACUAGGAUCUGGUUACCACUAAACCAGC
CUCAAGAACACCCGAAUGGAGUCUCUAAGCUACAUAAUACCAACUUACACUU
ACAAAAUGUUGUCCCCCAAAAUGUAGCCAUUCGUAUCUGCUCCUAAUAAAAA
GAAAGUUUCUUCACAUUCUAG
>mARM1888
(SEQ ID NO: 100)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUGGUCAACCUGCGCAUC
CUGCUGAACAACGCCGCCUUCCGCAACGGCCACAACUUCAUGGUGCGCAACU
UCCGCUGCGGCCAGCCCCUGCAGAACAAGGUGCAGCUGAAGGGCCGCGACCU
GCUGACCCUGAAGAACUUCACCGGCGAGGAGAUCAAGUACAUGCUGUGGCUG
AGCGCCGACCUGAAGUUCCGCAUCAAGCAGAAGGGCGAGUACCUGCCCCUGC
UGCAGGGCAAGAGCCUGGGCAUGAUCUUCGAGAAGCGCAGCACCCGCACCCG
CCUGAGCACCGAGACAGGCUUCGCCCUGCUGGGCGGCCACCCCUGCUUCCUG
ACCACCCAGGACAUCCACCUGGGCGUGAACGAGAGCCUGACCGACACCGCCC
GCGUGCUGAGCAGCAUGGCCGACGCCGUGCUGGCCCGCGUGUACAAGCAGAG
CGACCUGGACACCCUGGCCAAGGAGGCCAGCAUCCCCAUCAUCAACGGCCUG
AGCGACCUGUACCACCCCAUCCAGAUCCUGGCCGACUACCUGACCCUGCAGG
AGCACUACAGCAGCCUGAAGGGCCUGACCCUGAGCUGGAUCGGCGACGGCAA
CAACAUCCUGCACAGCAUCAUGAUGAGCGCCGCCAAGUUCGGCAUGCACCUG
CAGGCCGCCACCCCCAAGGGCUACGAGCCCGACGCCAGCGUGACCAAGCUGGC
CGAGCAGUACGCCAAGGAGAACGGCACCAAGCUGCUGCUGACCAACGACCCC
CUGGAGGCCGCCCACGGCGGCAACGUGCUGAUCACCGACACCUGGAUCAGCA
UGGGCCAGGAGGAGGAGAAGAAGAAGCGCCUGCAGGCCUUCCAGGGCUACCA
GGUGACCAUGAAGACCGCCAAGGUGGCCGCCAGCGACUGGACCUUCCUGCAC
UGCCUGCCCCGCAAGCCCGAGGAGGUGGACGACGAGGUGUUCUACAGCCCCC
GCAGCCUGGUGUUCCCCGAGGCCGAGAACCGCAAGUGGACCAUCAUGGCCGU
GAUGGUGAGCCUGCUGACCGACUACAGCCCCCAGCUGCAGAAGCCCAAGUUC
UGAUAACUCGAGCUAGUGACUGACUAGGAUCUGGUUACCACUAAACCAGCCU
CAAGAACACCCGAAUGGAGUCUCUAAGCUACAUAAUACCAACUUACACUUAC
AAAAUGUUGUCCCCCAAAAUGUAGCCAUUCGUAUCUGCUCCUAAUAAAAAGA
AAGUUUCUUCACAUUCUAGAAAAAAAAAAAAAAAAAAAAAAAAAAAAAAAA
AAAAAAAAAAAAAAAAAAAAAAAAAAAAAAAAAAAAAAAAAAAAAAAAAAA
AAAAAAAAAAAAAAAAA
>mARM1889
(SEQ ID NO: 101)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUGGUCAACCUGCGCAUC
CUGCUGAACAACGCCGCCUUCCGCAACGGCCACAACUUCAUGGUGCGCAACU
UCCGCUGCGGCCAGCCCCUGCAGAACCGGGUGCAGCUGAAGGGCCGCGACCU
GCUGACCCUGAAGAACUUCACCGGCGAGGAGAUCAAGUACAUGCUGUGGCUG
AGCGCCGACCUGAAGUUCCGCAUCAAGCAGAAGGGCGAGUACCUGCCCCUGC
UGCAGGGCAAGAGCCUGGGCAUGAUCUUCGAGAAGCGCAGCACCCGCACCCG
CCUGAGCACCGAGACAGGCUUCGCCCUGCUGGGCGGCCACCCCUGCUUCCUG
ACCACCCAGGACAUCCACCUGGGCGUGAACGAGAGCCUGACCGACACCGCCC
GCGUGCUGAGCAGCAUGGCCGACGCCGUGCUGGCCCGCGUGUACAAGCAGAG
CGACCUGGACACCCUGGCCAAGGAGGCCAGCAUCCCCAUCAUCAACGGCCUG
AGCGACCUGUACCACCCCAUCCAGAUCCUGGCCGACUACCUGACCCUGCAGG
AGCACUACAGCAGCCUGAAGGGCCUGACCCUGAGCUGGAUCGGCGACGGCAA
CAACAUCCUGCACAGCAUCAUGAUGAGCGCCGCCAAGUUCGGCAUGCACCUG
CAGGCCGCCACCCCCAAGGGCUACGAGCCCGACGCCAGCGUGACCAAGCUGGC
CGAGCAGUACGCCAAGGAGAACGGCACCAAGCUGCUGCUGACCAACGACCCC
CUGGAGGCCGCCCACGGCGGCAACGUGCUGAUCACCGACACCUGGAUCAGCA
UGGGCCAGGAGGAGGAGAAGAAGAAGCGCCUGCAGGCCUUCCAGGGCUACCA
GGUGACCAUGAAGACCGCCAAGGUGGCCGCCAGCGACUGGACCUUCCUGCAC
UGCCUGCCCCGCAAGCCCGAGGAGGUGGACGACGAGGUGUUCUACAGCCCCC
GCAGCCUGGUGUUCCCCGAGGCCGAGAACCGCAAGUGGACCAUCAUGGCCGU
GAUGGUGAGCCUGCUGACCGACUACAGCCCCCAGCUGCAGAAGCCCAAGUUC
UGAUAACUCGAGCUAGUGACUGACUAGGAUCUGGUUACCACUAAACCAGCCU
CAAGAACACCCGAAUGGAGUCUCUAAGCUACAUAAUACCAACUUACACUUAC
AAAAUGUUGUCCCCCAAAAUGUAGCCAUUCGUAUCUGCUCCUAAUAAAAAGA
AAGUUUCUUCACAUUCUAG
>mARM1890
(SEQ ID NO: 102)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUGUCAAUCUCCGCAUC
CUCCUUAACAACGCCGCGUUUAGAAACGGCCACAACUUCAUGGUCCGGAACU
UCAGAUGUGGCCAGCCGCUUCAAAACAAGGUCCAGCUGAAGGGCCGGGAUCU
UCUGACCCUGAAGAACUUUACUGGCGAAGAGAUCAAGUACAUGCUCUGGCUC
UCCGCGGACUUGAAGUUCCGCAUUAAGCAGAAGGGGGAAUACCUUCCGCUGC
UUCAAGGAAAGAGCCUCGGCAUGAUCUUUGAGAAGCGCUCAACCAGGACCCG
CCUUUCUACUGAAACUGGGUUCGCGCUGCUCGGUGGCCACCCCUGCUUCCUG
ACGACCCAGGACAUCCACCUCGGAGUGAACGAAUCCCUCACCGAUACCGCCC
GGGUGUUAUCGAGCAUGGCAGAUGCCGUGCUGGCCAGGGUGUACAAACAGUC
CGAUCUGGACACUCUGGCCAAGGAGGCGUCAAUUCCUAUUAUCAACGGCCUU
AGUGACCUCUACCAUCCGAUUCAGAUCCUGGCCGAUUACCUCACCCUGCAAG
AACACUACAGCUCCCUGAAGGGUCUGACAUUGUCCUGGAUCGGCGACGGCAA
CAACAUUCUCCAUUCCAUCAUGAUGUCCGCCGCAAAAUUCGGCAUGCAUCUU
CAAGCCGCCACGCCGAAGGGUUACGAGCCCGACGCUUCCGUGACUAAGCUCG
CCGAGCAGUACGCUAAGGAGAACGGAACCAAGCUUCUGCUGACUAACGACCC
ACUAGAAGCAGCCCACGGGGGCAACGUGCUUAUUACUGACACCUGGAUCUCC
AUGGGCCAGGAAGAAGAGAAAAAGAAGCGGCUGCAGGCGUUCCAGGGAUAU
CAGGUCACCAUGAAAACCGCCAAGGUCGCUGCCUCCGACUGGACCUUCCUGC
ACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCC
GUGAUGGUGUCACUGCUCACCGACUACAGCCCGCAGCUUCAGAAGCCCAAGU
UCUAGCUCGAGCUAGUGACUGACUAGGAUCUGGUUACCACUAAACCAGCCUC
AAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAA
AGUUUCUUCACAUUCUAG
>mARM1891
(SEQ ID NO: 103)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGCUUGUCAAUCUCCGCAUC
CUCCUUAACAACGCCGCGUUUAGAAACGGCCACAACUUCAUGGUCCGGAACU
UCAGAUGUGGCCAGCCGCUUCAAAACCGGGUCCAGCUGAAGGGCCGGGAUCU
UCUGACCCUGAAGAACUUUACUGGCGAAGAGAUCAAGUACAUGCUCUGGCUC
UCCGCGGACUUGAAGUUCCGCAUUAAGCAGAAGGGGGAAUACCUUCCGCUGC
UUCAAGGAAAGAGCCUCGGCAUGAUCUUUGAGAAGCGCUCAACCAGGACCCG
CCUUUCUACUGAAACUGGGUUCGCGCUGCUCGGUGGCCACCCCUGCUUCCUG
ACGACCCAGGACAUCCACCUCGGAGUGAACGAAUCCCUCACCGAUACCGCCC
GGGUGUUAUCGAGCAUGGCAGAUGCCGUGCUGGCCAGGGUGUACAAACAGUC
CGAUCUGGACACUCUGGCCAAGGAGGCGUCAAUUCCUAUUAUCAACGGCCUU
AGUGACCUCUACCAUCCGAUUCAGAUCCUGGCCGAUUACCUCACCCUGCAAG
AACACUACAGCUCCCUGAAGGGUCUGACAUUGUCCUGGAUCGGCGACGGCAA
CAACAUUCUCCAUUCCAUCAUGAUGUCCGCCGCAAAAUUCGGCAUGCAUCUU
CAAGCCGCCACGCCGAAGGGUUACGAGCCCGACGCUUCCGUGACUAAGCUCG
CCGAGCAGUACGCUAAGGAGAACGGAACCAAGCUUCUGCUGACUAACGACCC
ACUAGAAGCAGCCCACGGGGGCAACGUGCUUAUUACUGACACCUGGAUCUCC
AUGGGCCAGGAAGAAGAGAAAAAGAAGCGGCUGCAGGCGUUCCAGGGAUAU
CAGGUCACCAUGAAAACCGCCAAGGUCGCUGCCUCCGACUGGACCUUCCUGC
ACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCC
GUGAUGGUGUCACUGCUCACCGACUACAGCCCGCAGCUUCAGAAGCCCAAGU
UCUAGCUCGAGCUAGUGACUGACUAGGAUCUGGUUACCACUAAACCAGCCUC
AAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAA
AGUUUCUUCACAUUCUAG
>mARM1898
(SEQ ID NO: 104)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGGGCCUUGUCAAUCUCCGC
AUCCUCCUUAACAACGCCGCGUUUAGAAACGGCCACAACUUCAUGGUCCGGA
ACUUCAGAUGUGGCCAGCCGCUUCAAAACAAGGUCCAGCUGAAGGGCCGGGA
UCUUCUGACCCUGAAGAACUUUACUGGCGAAGAGAUCAAGUACAUGCUCUGG
CUCUCCGCGGACUUGAAGUUCCGCAUUAAGCAGAAGGGGGAAUACCUUCCGC
UGCUUCAAGGAAAGAGCCUCGGCAUGAUCUUUGAGAAGCGCUCAACCAGGAC
CCGCCUUUCUACUGAAACUGGGUUCGCGCUGCUCGGUGGCCACCCCUGCUUC
CUGACGACCCAGGACAUCCACCUCGGAGUGAACGAAUCCCUCACCGAUACCG
CCCGGGUGUUAUCGAGCAUGGCAGAUGCCGUGCUGGCCAGGGUGUACAAACA
GUCCGAUCUGGACACUCUGGCCAAGGAGGCGUCAAUUCCUAUUAUCAACGGC
CUUAGUGACCUCUACCAUCCGAUUCAGAUCCUGGCCGAUUACCUCACCCUGC
AAGAACACUACAGCUCCCUGAAGGGUCUGACAUUGUCCUGGAUCGGCGACGG
CAACAACAUUCUCCAUUCCAUCAUGAUGUCCGCCGCAAAAUUCGGCAUGCAU
CUUCAAGCCGCCACGCCGAAGGGUUACGAGCCCGACGCUUCCGUGACUAAGC
UCGCCGAGCAGUACGCUAAGGAGAACGGAACCAAGCUUCUGCUGACUAACGA
CCCACUAGAAGCAGCCCACGGGGGCAACGUGCUUAUUACUGACACCUGGAUC
UCCAUGGGCCAGGAAGAAGAGAAAAAGAAGCGGCUGCAGGCGUUCCAGGGAU
AUCAGGUCACCAUGAAAACCGCCAAGGUCGCUGCCUCCGACUGGACCUUCCU
GCACUGCCUGCCUCGCAAGCCUGAAGAAGUGGACGACGAGGUGUUCUACUCG
CCACGGAGCCUCGUGUUCCCCGAGGCCGAGAAUAGAAAGUGGACCAUCAUGG
CCGUGAUGGUGUCACUGCUCACCGACUACAGCCCGCAGCUUCAGAAGCCCAA
GUUCUAGCUCGAGCUAGUGACUGACUAGGAUCUGGUUACCACUAAACCAGCC
UCAAGAACACCCGAAUGGAGUCUCUAAGCUACAUAAUACCAACUUACACUUA
CAAAAUGUUGUCCCCCAAAAUGUAGCCAUUCGUAUCUGCUCCUAAUAAAAAG
AAAGUUUCUUCACAUUCUAG
>mARM1899
(SEQ ID NO: 105)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGGGCCUUGUCAAUCUCCGC
AUCCUCCUUAACAACGCCGCGUUUAGAAACGGCCACAACUUCAUGGUCCGGA
ACUUCAGAUGUGGCCAGCCGCUUCAAAACCGGGUCCAGCUGAAGGGCCGGGA
UCUUCUGACCCUGAAGAACUUUACUGGCGAAGAGAUCAAGUACAUGCUCUGG
CUCUCCGCGGACUUGAAGUUCCGCAUUAAGCAGAAGGGGGAAUACCUUCCGC
UGCUUCAAGGAAAGAGCCUCGGCAUGAUCUUUGAGAAGCGCUCAACCAGGAC
CCGCCUUUCUACUGAAACUGGGUUCGCGCUGCUCGGUGGCCACCCCUGCUUC
CUGACGACCCAGGACAUCCACCUCGGAGUGAACGAAUCCCUCACCGAUACCG
CCCGGGUGUUAUCGAGCAUGGCAGAUGCCGUGCUGGCCAGGGUGUACAAACA
GUCCGAUCUGGACACUCUGGCCAAGGAGGCGUCAAUUCCUAUUAUCAACGGC
CUUAGUGACCUCUACCAUCCGAUUCAGAUCCUGGCCGAUUACCUCACCCUGC
AAGAACACUACAGCUCCCUGAAGGGUCUGACAUUGUCCUGGAUCGGCGACGG
CAACAACAUUCUCCAUUCCAUCAUGAUGUCCGCCGCAAAAUUCGGCAUGCAU
CUUCAAGCCGCCACGCCGAAGGGUUACGAGCCCGACGCUUCCGUGACUAAGC
UCGCCGAGCAGUACGCUAAGGAGAACGGAACCAAGCUUCUGCUGACUAACGA
CCCACUAGAAGCAGCCCACGGGGGCAACGUGCUUAUUACUGACACCUGGAUC
UCCAUGGGCCAGGAAGAAGAGAAAAAGAAGCGGCUGCAGGCGUUCCAGGGAU
AUCAGGUCACCAUGAAAACCGCCAAGGUCGCUGCCUCCGACUGGACCUUCCU
GCACUGCCUGCCUCGCAAGCCUGAAGAAGUGGACGACGAGGUGUUCUACUCG
CCACGGAGCCUCGUGUUCCCCGAGGCCGAGAAUAGAAAGUGGACCAUCAUGG
CCGUGAUGGUGUCACUGCUCACCGACUACAGCCCGCAGCUUCAGAAGCCCAA
GUUCUAGCUCGAGCUAGUGACUGACUAGGAUCUGGUUACCACUAAACCAGCC
UCAAGAACACCCGAAUGGAGUCUCUAAGCUACAUAAUACCAACUUACACUUA
CAAAAUGUUGUCCCCCAAAAUGUAGCCAUUCGUAUCUGCUCCUAAUAAAAAG
AAAGUUUCUUCACAUUCUAGAAAAAAAAAAAAAAAAAAAAAAAAAAAAAAA
AAAAAAAAAAAAAAAAAAAAAAAAAAAAAAAAAAAAAAAAAAAAAAAAAAA
AAAAAAAAAAAAAAAAAAAAAAAAAAAAAAAA
>mARM1900
(SEQ ID NO: 106)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGGGCGGACUUGUCAAUCUC
CGCAUCCUCCUUAACAACGCCGCGUUUAGAAACGGCCACAACUUCAUGGUCC
GGAACUUCAGAUGUGGCCAGCCGCUUCAAAACAAGGUCCAGCUGAAGGGCCG
GGAUCUUCUGACCCUGAAGAACUUUACUGGCGAAGAGAUCAAGUACAUGCUC
UGGCUCUCCGCGGACUUGAAGUUCCGCAUUAAGCAGAAGGGGGAAUACCUUC
CGCUGCUUCAAGGAAAGAGCCUCGGCAUGAUCUUUGAGAAGCGCUCAACCAG
GACCCGCCUUUCUACUGAAACUGGGUUCGCGCUGCUCGGUGGCCACCCCUGC
UUCCUGACGACCCAGGACAUCCACCUCGGAGUGAACGAAUCCCUCACCGAUA
CCGCCCGGGUGUUAUCGAGCAUGGCAGAUGCCGUGCUGGCCAGGGUGUACAA
ACAGUCCGAUCUGGACACUCUGGCCAAGGAGGCGUCAAUUCCUAUUAUCAAC
GGCCUUAGUGACCUCUACCAUCCGAUUCAGAUCCUGGCCGAUUACCUCACCC
UGCAAGAACACUACAGCUCCCUGAAGGGUCUGACAUUGUCCUGGAUCGGCGA
CGGCAACAACAUUCUCCAUUCCAUCAUGAUGUCCGCCGCAAAAUUCGGCAUG
CAUCUUCAAGCCGCCACGCCGAAGGGUUACGAGCCCGACGCUUCCGUGACUA
AGCUCGCCGAGCAGUACGCUAAGGAGAACGGAACCAAGCUUCUGCUGACUAA
CGACCCACUAGAAGCAGCCCACGGGGGCAACGUGCUUAUUACUGACACCUGG
AUCUCCAUGGGCCAGGAAGAAGAGAAAAAGAAGCGGCUGCAGGCGUUCCAGG
GAUAUCAGGUCACCAUGAAAACCGCCAAGGUCGCUGCCUCCGACUGGACCUU
CCUGCACUGCCUGCCUCGCAAGCCUGAAGAAGUGGACGACGAGGUGUUCUAC
UCGCCACGGAGCCUCGUGUUCCCCGAGGCCGAGAAUAGAAAGUGGACCAUCA
UGGCCGUGAUGGUGUCACUGCUCACCGACUACAGCCCGCAGCUUCAGAAGCC
CAAGUUCUAGCUCGAGCUAGUGACUGACUAGGAUCUGGUUACCACUAAACCA
GCCUCAAGAACACCCGAAUGGAGUCUCUAAGCUACAUAAUACCAACUUACAC
UUACAAAAUGUUGUCCCCCAAAAUGUAGCCAUUCGUAUCUGCUCCUAAUAAA
AAGAAAGUUUCUUCACAUUCUAG
>mARM1903
(SEQ ID NO: 107)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCAUGGCCCUUUUCAAUCUCCGCAUC
CUCCUUAACAACGCCGCGUUUAGAAACGGCCACAACUUCAUGGUCCGGAACU
UCAGAUGUGGCCAGCCGCUUCAAGGCAAGGUCCAGCUGAAGGGCCGGGAUCU
UCUGACCCUGAAGAACUUUACUGGCGAAGAGAUCAAGUACAUGCUCUGGCUC
UCCGCGGACUUGAAGUUCCGCAUUAAGCAGAAGGGGGAAUACCUUCCGCUGC
UUCAAGGAAAGAGCCUCGGCAUGAUCUUUGAGAAGCGCUCAACCAGGACCCG
CCUUUCUACUGAAACUGGGUUCGCGCUGCUCGGUGGCCACCCCUGCUUCCUG
ACGACCCAGGACAUCCACCUCGGAGUGAACGAAUCCCUCACCGAUACCGCCC
GGGUGUUAUCGAGCAUGGCAGAUGCCGUGCUGGCCAGGGUGUACAAACAGUC
CGAUCUGGACACUCUGGCCAAGGAGGCGUCAAUUCCUAUUAUCAACGGCCUU
AGUGACCUCUACCAUCCGAUUCAGAUCCUGGCCGAUUACCUCACCCUGCAAG
AACACUACAGCUCCCUGAAGGGUCUGACAUUGUCCUGGAUCGGCGACGGCAA
CAACAUUCUCCAUUCCAUCAUGAUGUCCGCCGCAAAAUUCGGCAUGCAUCUU
CAAGCCGCCACGCCGAAGGGUUACGAGCCCGACGCUUCCGUGACUAAGCUCG
CCGAGCAGUACGCUAAGGAGAACGGAACCAAGCUUCUGCUGACUAACGACCC
ACUAGAAGCAGCCCACGGGGGCAACGUGCUUAUUACUGACACCUGGAUCUCC
AUGGGCCAGGAAGAAGAGAAAAAGAAGCGGCUGCAGGCGUUCCAGGGAUAU
CAGGUCACCAUGAAAACCGCCAAGGUCGCUGCCUCCGACUGGACCUUCCUGC
ACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCC
GUGAUGGUGUCACUGCUCACCGACUACAGCCCGCAGCUUCAGAAGCCCAAGU
UCUAGAUAAGUGAACUCGAGCUAGUGACUGACUAGGAUCUGGUUACCACUAA
ACCAGCCUCAAGAACACCCGAAUGGAGUCUCUAAGCUACAUAAUACCAACUU
ACACUUACAAAAUGUUGUCCCCCAAAAUGUAGCCAUUCGUAUCUGCUCCUAA
UAAAAAGAAAGUUUCUUCACAUUCUAG
>mARM1904
(SEQ ID NO: 108)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCAUGGCCCUUUUCAAUCUCCGCAUC
CUCCUUAACAACGCCGCGUUUAGAAACGGCCACAACUUCAUGGUCCGGAACU
UCAGAUGUGGCCAGCCGCUUCAAGGCCGGGUCCAGCUGAAGGGCCGGGAUCU
UCUGACCCUGAAGAACUUUACUGGCGAAGAGAUCAAGUACAUGCUCUGGCUC
UCCGCGGACUUGAAGUUCCGCAUUAAGCAGAAGGGGGAAUACCUUCCGCUGC
UUCAAGGAAAGAGCCUCGGCAUGAUCUUUGAGAAGCGCUCAACCAGGACCCG
CCUUUCUACUGAAACUGGGUUCGCGCUGCUCGGUGGCCACCCCUGCUUCCUG
ACGACCCAGGACAUCCACCUCGGAGUGAACGAAUCCCUCACCGAUACCGCCC
GGGUGUUAUCGAGCAUGGCAGAUGCCGUGCUGGCCAGGGUGUACAAACAGUC
CGAUCUGGACACUCUGGCCAAGGAGGCGUCAAUUCCUAUUAUCAACGGCCUU
AGUGACCUCUACCAUCCGAUUCAGAUCCUGGCCGAUUACCUCACCCUGCAAG
AACACUACAGCUCCCUGAAGGGUCUGACAUUGUCCUGGAUCGGCGACGGCAA
CAACAUUCUCCAUUCCAUCAUGAUGUCCGCCGCAAAAUUCGGCAUGCAUCUU
CAAGCCGCCACGCCGAAGGGUUACGAGCCCGACGCUUCCGUGACUAAGCUCG
CCGAGCAGUACGCUAAGGAGAACGGAACCAAGCUUCUGCUGACUAACGACCC
ACUAGAAGCAGCCCACGGGGGCAACGUGCUUAUUACUGACACCUGGAUCUCC
AUGGGCCAGGAAGAAGAGAAAAAGAAGCGGCUGCAGGCGUUCCAGGGAUAU
CAGGUCACCAUGAAAACCGCCAAGGUCGCUGCCUCCGACUGGACCUUCCUGC
ACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCC
GUGAUGGUGUCACUGCUCACCGACUACAGCCCGCAGCUUCAGAAGCCCAAGU
UCUAGAUAAGUGAACUCGAGCUAGUGACUGACUAGGAUCUGGUUACCACUAA
ACCAGCCUCAAGAACACCCGAAUGGAGUCUCUAAGCUACAUAAUACCAACUU
ACACUUACAAAAUGUUGUCCCCCAAAAUGUAGCCAUUCGUAUCUGCUCCUAA
UAAAAAGAAAGUUUCUUCACAUUCUAG
>mARM1905
(SEQ ID NO: 109)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCAUGGCCCUUUUCAAUCUCCGCAUC
CUCCUUAACAACGCCGCGUUUAGAAACGGCCACAACUUCAUGGUCCGGAACU
UCAGAUGUGGCCAGCCGCUUCAAGGCCGGGUCCAGCUGAAGGGCCGGGAUCU
UCUGACCCUGAAGAACUUUACUGGCGAAGAGAUCAGGUACAUGCUCUGGCUC
UCCGCGGACUUGAAGUUCCGCAUUAAGCAGAAGGGGGAAUACCUUCCGCUGC
UUCAAGGAAAGAGCCUCGGCAUGAUCUUUGAGAAGCGCUCAACCAGGACCCG
CCUUUCUACUGAAACUGGGUUCGCGCUGCUCGGUGGCCACCCCUGCUUCCUG
ACGACCCAGGACAUCCACCUCGGAGUGAACGAAUCCCUCACCGAUACCGCCC
GGGUGUUAUCGAGCAUGGCAGAUGCCGUGCUGGCCAGGGUGUACAAACAGUC
CGAUCUGGACACUCUGGCCAAGGAGGCGUCAAUUCCUAUUAUCAACGGCCUU
AGUGACCUCUACCAUCCGAUUCAGAUCCUGGCCGAUUACCUCACCCUGCAAG
AACACUACAGCUCCCUGAAGGGUCUGACAUUGUCCUGGAUCGGCGACGGCAA
CAACAUUCUCCAUUCCAUCAUGAUGUCCGCCGCAAAAUUCGGCAUGCAUCUU
CAAGCCGCCACGCCGAAGGGUUACGAGCCCGACGCUUCCGUGACUAAGCUCG
CCGAGCAGUACGCUAAGGAGAACGGAACCAAGCUUCUGCUGACUAACGACCC
ACUAGAAGCAGCCCACGGGGGCAACGUGCUUAUUACUGACACCUGGAUCUCC
AUGGGCCAGGAAGAAGAGAAAAAGAAGCGGCUGCAGGCGUUCCAGGGAUAU
CAGGUCACCAUGAAAACCGCCAAGGUCGCUGCCUCCGACUGGACCUUCCUGC
ACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCC
GUGAUGGUGUCACUGCUCACCGACUACAGCCCGCAGCUUCAGAAGCCCAAGU
UCUAGAUAAGUGAACUCGAGCUAGUGACUGACUAGGAUCUGGUUACCACUAA
ACCAGCCUCAAGAACACCCGAAUGGAGUCUCUAAGCUACAUAAUACCAACUU
ACACUUACAAAAUGUUGUCCCCCAAAAUGUAGCCAUUCGUAUCUGCUCCUAA
UAAAAAGAAAGUUUCUUCACAUUCUAG
>mARM1906
(SEQ ID NO: 110)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCAUGGCCCUUGUCAAUCUCCGCAUC
CUCCUUAACAACGCCGCGUUUAGAAACGGCCACAACUUCAUGGUCCGGAACU
UCAGAUGUGGCCAGCCGCUUCAAGGCAGGGUCCAGCUGAAGGGCCGGGAUCU
UCUGACCCUGAAGAACUUUACUGGCGAAGAGAUCAAGUACAUGCUCUGGCUC
UCCGCGGACUUGAAGUUCCGCAUUAAGCAGAAGGGGGAAUACCUUCCGCUGC
UUCAAGGAAAGAGCCUCGGCAUGAUCUUUGAGAAGCGCUCAACCAGGACCCG
CCUUUCUACUGAAACUGGGUUCGCGCUGCUCGGUGGCCACCCCUGCUUCCUG
ACGACCCAGGACAUCCACCUCGGAGUGAACGAAUCCCUCACCGAUACCGCCC
GGGUGUUAUCGAGCAUGGCAGAUGCCGUGCUGGCCAGGGUGUACAAACAGUC
CGAUCUGGACACUCUGGCCAAGGAGGCGUCAAUUCCUAUUAUCAACGGCCUU
AGUGACCUCUACCAUCCGAUUCAGAUCCUGGCCGAUUACCUCACCCUGCAAG
AACACUACAGCUCCCUGAAGGGUCUGACAUUGUCCUGGAUCGGCGACGGCAA
CAACAUUCUCCAUUCCAUCAUGAUGUCCGCCGCAAAAUUCGGCAUGCAUCUU
CAAGCCGCCACGCCGAAGGGUUACGAGCCCGACGCUUCCGUGACUAAGCUCG
CCGAGCAGUACGCUAAGGAGAACGGAACCAAGCUUCUGCUGACUAACGACCC
ACUAGAAGCAGCCCACGGGGGCAACGUGCUUAUUACUGACACCUGGAUCUCC
AUGGGCCAGGAAGAAGAGAAAAAGAAGCGGCUGCAGGCGUUCCAGGGAUAU
CAGGUCACCAUGAAAACCGCCAAGGUCGCUGCCUCCGACUGGACCUUCCUGC
ACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCC
GUGAUGGUGUCACUGCUCACCGACUACAGCCCGCAGCUUCAGAAGCCCAAGU
UCUAAGUGAAUAGACUCGAGCUAGUGACUGACUAGGAUCUGGUUACCACUAA
ACCAGCCUCAAGAACACCCGAAUGGAGUCUCUAAGCUACAUAAUACCAACUU
ACACUUACAAAAUGUUGUCCCCCAAAAUGUAGCCAUUCGUAUCUGCUCCUAA
UAAAAAGAAAGUUUCUUCACAUUCUAG
>mARM1907
(SEQ ID NO: 111)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCAUGGCCCUUUUCAAUCUCCGCAUC
CUCCUUAACAACGCCGCGUUUAGAAACGGCCACAACUUCAUGGUCCGGAACU
UCAGAUGUGGCCAGCCGCUUCAAGUCAAGGUCCAGCUGAAGGGCCGGGAUCU
UCUGACCCUGAAGAACUUUACUGGCGAAGAGAUCAAGUACAUGCUCUGGCUC
UCCGCGGACUUGAAGUUCCGCAUUAAGCAGAAGGGGGAAUACCUUCCGCUGC
UUCAAGGAAAGAGCCUCGGCAUGAUCUUUGAGAAGCGCUCAACCAGGACCCG
CCUUUCUACUGAAACUGGGUUCGCGCUGCUCGGUGGCCACCCCUGCUUCCUG
ACGACCCAGGACAUCCACCUCGGAGUGAACGAAUCCCUCACCGAUACCGCCC
GGGUGUUAUCGAGCAUGGCAGAUGCCGUGCUGGCCAGGGUGUACAAACAGUC
CGAUCUGGACACUCUGGCCAAGGAGGCGUCAAUUCCUAUUAUCAACGGCCUU
AGUGACCUCUACCAUCCGAUUCAGAUCCUGGCCGAUUACCUCACCCUGCAAG
AACACUACAGCUCCCUGAAGGGUCUGACAUUGUCCUGGAUCGGCGACGGCAA
CAACAUUCUCCAUUCCAUCAUGAUGUCCGCCGCAAAAUUCGGCAUGCAUCUU
CAAGCCGCCACGCCGAAGGGUUACGAGCCCGACGCUUCCGUGACUAAGCUCG
CCGAGCAGUACGCUAAGGAGAACGGAACCAAGCUUCUGCUGACUAACGACCC
ACUAGAAGCAGCCCACGGGGGCAACGUGCUUAUUACUGACACCUGGAUCUCC
AUGGGCCAGGAAGAAGAGAAAAAGAAGCGGCUGCAGGCGUUCCAGGGAUAU
CAGGUCACCAUGAAAACCGCCAAGGUCGCUGCCUCCGACUGGACCUUCCUGC
ACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCC
GUGAUGGUGUCACUGCUCACCGACUACAGCCCGCAGCUUCAGAAGCCCAAGU
UCUAGAUAAGUGAACUCGAGCUAGUGACUGACUAGGAUCUGGUUACCACUAA
ACCAGCCUCAAGAACACCCGAAUGGAGUCUCUAAGCUACAUAAUACCAACUU
ACACUUACAAAAUGUUGUCCCCCAAAAUGUAGCCAUUCGUAUCUGCUCCUAA
UAAAAAGAAAGUUUCUUCACAUUCUAGAAAAAAAAAAAAAAAAAAAAAAAA
AAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAA
>mARM1908
(SEQ ID NO: 112)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCAUGGCCCUUUUCAAUCUCCGCAUC
CUCCUUAACAACGCCGCGUUUAGAAACGGCCACAACUUCAUGGUCCGGAACU
UCAGAUGUGGCCAGCCGCUUCAAGUCAGGGUCCAGCUGAAGGGCCGGGAUCU
UCUGACCCUGAAGAACUUUACUGGCGAAGAGAUCAAGUACAUGCUCUGGCUC
UCCGCGGACUUGAAGUUCCGCAUUAAGCAGAAGGGGGAAUACCUUCCGCUGC
UUCAAGGAAAGAGCCUCGGCAUGAUCUUUGAGAAGCGCUCAACCAGGACCCG
CCUUUCUACUGAAACUGGGUUCGCGCUGCUCGGUGGCCACCCCUGCUUCCUG
ACGACCCAGGACAUCCACCUCGGAGUGAACGAAUCCCUCACCGAUACCGCCC
GGGUGUUAUCGAGCAUGGCAGAUGCCGUGCUGGCCAGGGUGUACAAACAGUC
CGAUCUGGACACUCUGGCCAAGGAGGCGUCAAUUCCUAUUAUCAACGGCCUU
AGUGACCUCUACCAUCCGAUUCAGAUCCUGGCCGAUUACCUCACCCUGCAAG
AACACUACAGCUCCCUGAAGGGUCUGACAUUGUCCUGGAUCGGCGACGGCAA
CAACAUUCUCCAUUCCAUCAUGAUGUCCGCCGCAAAAUUCGGCAUGCAUCUU
CAAGCCGCCACGCCGAAGGGUUACGAGCCCGACGCUUCCGUGACUAAGCUCG
CCGAGCAGUACGCUAAGGAGAACGGAACCAAGCUUCUGCUGACUAACGACCC
ACUAGAAGCAGCCCACGGGGGCAACGUGCUUAUUACUGACACCUGGAUCUCC
AUGGGCCAGGAAGAAGAGAAAAAGAAGCGGCUGCAGGCGUUCCAGGGAUAU
CAGGUCACCAUGAAAACCGCCAAGGUCGCUGCCUCCGACUGGACCUUCCUGC
ACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCC
GUGAUGGUGUCACUGCUCACCGACUACAGCCCGCAGCUUCAGAAGCCCAAGU
UCUAGAUAAGUGAACUCGAGCUAGUGACUGACUAGGAUCUGGUUACCACUAA
ACCAGCCUCAAGAACACCCGAAUGGAGUCUCUAAGCUACAUAAUACCAACUU
ACACUUACAAAAUGUUGUCCCCCAAAAUGUAGCCAUUCGUAUCUGCUCCUAA
UAAAAAGAAAGUUUCUUCACAUUCUAG
>mARM1915
(SEQ ID NO: 113)
GGCAGAAAAAUUUGCUACAUUGUUUCACAAACUUCAAAUAUUAUUCAUUUA
UUUAGAUCUAUUAUUACAUCAAAACAAAAAGCCGCCACCAUGCUGUUCAACC
UGCGCAUCCUGCUGAACAACGCCGCCUUCCGCAACGGCCACAACUUCAUGGU
GCGCAACUUCCGCUGCGGCCAGCCCCUGCAGAACAAGGUGCAGCUGAAGGGC
CGCGACCUGCUGACCCUGAAGAACUUCACCGGCGAGGAGAUCAAGUACAUGC
UGUGGCUGAGCGCCGACCUGAAGUUCCGCAUCAAGCAGAAGGGCGAGUACCU
GCCCCUGCUGCAGGGCAAGAGCCUGGGCAUGAUCUUCGAGAAGCGCAGCACC
CGCACCCGCCUGAGCACCGAGACAGGCUUCGCCCUGCUGGGCGGCCACCCCUG
CUUCCUGACCACCCAGGACAUCCACCUGGGCGUGAACGAGAGCCUGACCGAC
ACCGCCCGCGUGCUGAGCAGCAUGGCCGACGCCGUGCUGGCCCGCGUGUACA
AGCAGAGCGACCUGGACACCCUGGCCAAGGAGGCCAGCAUCCCCAUCAUCAA
CGGCCUGAGCGACCUGUACCACCCCAUCCAGAUCCUGGCCGACUACCUGACCC
UGCAGGAGCACUACAGCAGCCUGAAGGGCCUGACCCUGAGCUGGAUCGGCGA
CGGCAACAACAUCCUGCACAGCAUCAUGAUGAGCGCCGCCAAGUUCGGCAUG
CACCUGCAGGCCGCCACCCCCAAGGGCUACGAGCCCGACGCCAGCGUGACCAA
GCUGGCCGAGCAGUACGCCAAGGAGAACGGCACCAAGCUGCUGCUGACCAAC
GACCCCCUGGAGGCCGCCCACGGCGGCAACGUGCUGAUCACCGACACCUGGA
UCAGCAUGGGCCAGGAGGAGGAGAAGAAGAAGCGCCUGCAGGCCUUCCAGGG
CUACCAGGUGACCAUGAAGACCGCCAAGGUGGCCGCCAGCGACUGGACCUUC
CUGCACUGCCUGCCCCGCAAGCCCGAGGAGGUGGACGACGAGGUGUUCUACA
GCCCCCGCAGCCUGGUGUUCCCCGAGGCCGAGAACCGCAAGUGGACCAUCAU
GGCCGUGAUGGUGAGCCUGCUGACCGACUACAGCCCCCAGCUGCAGAAGCCC
AAGUUCUGAGGUCUCUAGUAAUGAGCUGGAGCCUCGGUAGCCGUUCCUCCUG
CCCGCUGGGCCUCCCAACGGGCCCUCCUCCCCUCCUUGCACCGGCCCUUCCUG
GUCUUUGAAUAAAGUCUGAGUGGGCAUCUAG
>mARM1916
(SEQ ID NO: 114)
GGCAGAAAAAUUUGCUACAUUGUUUCACAAACUUCAAAUAUUAUUCAUUUA
UUUAGAUCUAUUAUUACAUCAAAACAAAAAGCCGCCACCAUGGGAGUAUUCA
ACCUGCGCAUCCUGCUGAACAACGCCGCCUUCCGCAACGGCCACAACUUCAU
GGUGCGCAACUUCCGCUGCGGCCAGCCCCUGCAGAACAAGGUGCAGCUGAAG
GGCCGCGACCUGCUGACCCUGAAGAACUUCACCGGCGAGGAGAUCAAGUACA
UGCUGUGGCUGAGCGCCGACCUGAAGUUCCGCAUCAAGCAGAAGGGCGAGUA
CCUGCCCCUGCUGCAGGGCAAGAGCCUGGGCAUGAUCUUCGAGAAGCGCAGC
ACCCGCACCCGCCUGAGCACCGAGACAGGCUUCGCCCUGCUGGGCGGCCACCC
CUGCUUCCUGACCACCCAGGACAUCCACCUGGGCGUGAACGAGAGCCUGACC
GACACCGCCCGCGUGCUGAGCAGCAUGGCCGACGCCGUGCUGGCCCGCGUGU
ACAAGCAGAGCGACCUGGACACCCUGGCCAAGGAGGCCAGCAUCCCCAUCAU
CAACGGCCUGAGCGACCUGUACCACCCCAUCCAGAUCCUGGCCGACUACCUG
ACCCUGCAGGAGCACUACAGCAGCCUGAAGGGCCUGACCCUGAGCUGGAUCG
GCGACGGCAACAACAUCCUGCACAGCAUCAUGAUGAGCGCCGCCAAGUUCGG
CAUGCACCUGCAGGCCGCCACCCCCAAGGGCUACGAGCCCGACGCCAGCGUGA
CCAAGCUGGCCGAGCAGUACGCCAAGGAGAACGGCACCAAGCUGCUGCUGAC
CAACGACCCCCUGGAGGCCGCCCACGGCGGCAACGUGCUGAUCACCGACACCU
GGAUCAGCAUGGGCCAGGAGGAGGAGAAGAAGAAGCGCCUGCAGGCCUUCCA
GGGCUACCAGGUGACCAUGAAGACCGCCAAGGUGGCCGCCAGCGACUGGACC
UUCCUGCACUGCCUGCCCCGCAAGCCCGAGGAGGUGGACGACGAGGUGUUCU
ACAGCCCCCGCAGCCUGGUGUUCCCCGAGGCCGAGAACCGCAAGUGGACCAU
CAUGGCCGUGAUGGUGAGCCUGCUGACCGACUACAGCCCCCAGCUGCAGAAG
CCCAAGUUCUGAGGUCUCUAGUAAUGAGCUGGAGCCUCGGUAGCCGUUCCUC
CUGCCCGCUGGGCCUCCCAACGGGCCCUCCUCCCCUCCUUGCACCGGCCCUUC
CUGGUCUUUGAAUAAAGUCUGAGUGGGCAUCUAG
>mARM1917
(SEQ ID NO: 115)
GGCAGAAAAAUUUGCUACAUUGUUUCACAAACUUCAAAUAUUAUUCAUUUA
UUUAGAUCUAUUAUUACAUCAAAACAAAAAGCCGCCACCAUGGGAGUAUUCA
ACCUGCGCAUCCUGCUGAACAACGCCGCCUUCCGCAACGGCCACAACUUCAU
GGUGCGCAACUUCCGCUGCGGCCAGCCCCUGCAGAACCGGGUGCAGCUGAAG
GGCCGCGACCUGCUGACCCUGAAGAACUUCACCGGCGAGGAGAUCCGGUACA
UGCUGUGGCUGAGCGCCGACCUGAAGUUCCGCAUCAAGCAGAAGGGCGAGUA
CCUGCCCCUGCUGCAGGGCAAGAGCCUGGGCAUGAUCUUCGAGAAGCGCAGC
ACCCGCACCCGCCUGAGCACCGAGACAGGCUUCGCCCUGCUGGGCGGCCACCC
CUGCUUCCUGACCACCCAGGACAUCCACCUGGGCGUGAACGAGAGCCUGACC
GACACCGCCCGCGUGCUGAGCAGCAUGGCCGACGCCGUGCUGGCCCGCGUGU
ACAAGCAGAGCGACCUGGACACCCUGGCCAAGGAGGCCAGCAUCCCCAUCAU
CAACGGCCUGAGCGACCUGUACCACCCCAUCCAGAUCCUGGCCGACUACCUG
ACCCUGCAGGAGCACUACAGCAGCCUGAAGGGCCUGACCCUGAGCUGGAUCG
GCGACGGCAACAACAUCCUGCACAGCAUCAUGAUGAGCGCCGCCAAGUUCGG
CAUGCACCUGCAGGCCGCCACCCCCAAGGGCUACGAGCCCGACGCCAGCGUGA
CCAAGCUGGCCGAGCAGUACGCCAAGGAGAACGGCACCAAGCUGCUGCUGAC
CAACGACCCCCUGGAGGCCGCCCACGGCGGCAACGUGCUGAUCACCGACACCU
GGAUCAGCAUGGGCCAGGAGGAGGAGAAGAAGAAGCGCCUGCAGGCCUUCCA
GGGCUACCAGGUGACCAUGAAGACCGCCAAGGUGGCCGCCAGCGACUGGACC
UUCCUGCACUGCCUGCCCCGCAAGCCCGAGGAGGUGGACGACGAGGUGUUCU
ACAGCCCCCGCAGCCUGGUGUUCCCCGAGGCCGAGAACCGCAAGUGGACCAU
CAUGGCCGUGAUGGUGAGCCUGCUGACCGACUACAGCCCCCAGCUGCAGAAG
CCCAAGUUCUGAGGUCUCUAGUAAUGAGCUGGAGCCUCGGUAGCCGUUCCUC
CUGCCCGCUGGGCCUCCCAACGGGCCCUCCUCCCCUCCUUGCACCGGCCCUUC
CUGGUCUUUGAAUAAAGUCUGAGUGGGCAUCUAG
>mARM1918
(SEQ ID NO: 116)
GGCAGAAAAAUUUGCUACAUUGUUUCACAAACUUCAAAUAUUAUUCAUUUA
UUUAGAUCUAUUAUUACAUCAAAACAAAAAGCCGCCACCAUGCUGGUAUUCA
ACCUGCGCAUCCUGCUGAACAACGCCGCCUUCCGCAACGGCCACAACUUCAU
GGUGCGCAACUUCCGCUGCGGCCAGCCCCUGCAGAACCGGGUGCAGCUGAAG
GGCCGCGACCUGCUGACCCUGAAGAACUUCACCGGCGAGGAGAUCCGGUACA
UGCUGUGGCUGAGCGCCGACCUGAAGUUCCGCAUCAAGCAGAAGGGCGAGUA
CCUGCCCCUGCUGCAGGGCAAGAGCCUGGGCAUGAUCUUCGAGAAGCGCAGC
ACCCGCACCCGCCUGAGCACCGAGACAGGCUUCGCCCUGCUGGGCGGCCACCC
CUGCUUCCUGACCACCCAGGACAUCCACCUGGGCGUGAACGAGAGCCUGACC
GACACCGCCCGCGUGCUGAGCAGCAUGGCCGACGCCGUGCUGGCCCGCGUGU
ACAAGCAGAGCGACCUGGACACCCUGGCCAAGGAGGCCAGCAUCCCCAUCAU
CAACGGCCUGAGCGACCUGUACCACCCCAUCCAGAUCCUGGCCGACUACCUG
ACCCUGCAGGAGCACUACAGCAGCCUGAAGGGCCUGACCCUGAGCUGGAUCG
GCGACGGCAACAACAUCCUGCACAGCAUCAUGAUGAGCGCCGCCAAGUUCGG
CAUGCACCUGCAGGCCGCCACCCCCAAGGGCUACGAGCCCGACGCCAGCGUGA
CCAAGCUGGCCGAGCAGUACGCCAAGGAGAACGGCACCAAGCUGCUGCUGAC
CAACGACCCCCUGGAGGCCGCCCACGGCGGCAACGUGCUGAUCACCGACACCU
GGAUCAGCAUGGGCCAGGAGGAGGAGAAGAAGAAGCGCCUGCAGGCCUUCCA
GGGCUACCAGGUGACCAUGAAGACCGCCAAGGUGGCCGCCAGCGACUGGACC
UUCCUGCACUGCCUGCCCCGCAAGCCCGAGGAGGUGGACGACGAGGUGUUCU
ACAGCCCCCGCAGCCUGGUGUUCCCCGAGGCCGAGAACCGCAAGUGGACCAU
CAUGGCCGUGAUGGUGAGCCUGCUGACCGACUACAGCCCCCAGCUGCAGAAG
CCCAAGUUCUGAGGUCUCUAGUAAUGAGCUGGAGCCUCGGUAGCCGUUCCUC
CUGCCCGCUGGGCCUCCCAACGGGCCCUCCUCCCCUCCUUGCACCGGCCCUUC
CUGGUCUUUGAAUAAAGUCUGAGUGGGCAUCUAG
>mARM1919
(SEQ ID NO: 117)
AUUAUUACAUCAAAACAAAAAGCCGCCACCAUGGGAGUAUUCAACCUGCGCA
UCCUGCUGAACAACGCCGCCUUCCGCAACGGCCACAACUUCAUGGUGCGCAA
CUUCCGCUGCGGCCAGCCCCUGCAGAACAAGGUGCAGCUGAAGGGCCGCGAC
CUGCUGACCCUGAAGAACUUCACCGGCGAGGAGAUCAAGUACAUGCUGUGGC
UGAGCGCCGACCUGAAGUUCCGCAUCAAGCAGAAGGGCGAGUACCUGCCCCU
GCUGCAGGGCAAGAGCCUGGGCAUGAUCUUCGAGAAGCGCAGCACCCGCACC
CGCCUGAGCACCGAGACAGGCUUCGCCCUGCUGGGCGGCCACCCCUGCUUCCU
GACCACCCAGGACAUCCACCUGGGCGUGAACGAGAGCCUGACCGACACCGCC
CGCGUGCUGAGCAGCAUGGCCGACGCCGUGCUGGCCCGCGUGUACAAGCAGA
GCGACCUGGACACCCUGGCCAAGGAGGCCAGCAUCCCCAUCAUCAACGGCCU
GAGCGACCUGUACCACCCCAUCCAGAUCCUGGCCGACUACCUGACCCUGCAG
GAGCACUACAGCAGCCUGAAGGGCCUGACCCUGAGCUGGAUCGGCGACGGCA
ACAACAUCCUGCACAGCAUCAUGAUGAGCGCCGCCAAGUUCGGCAUGCACCU
GCAGGCCGCCACCCCCAAGGGCUACGAGCCCGACGCCAGCGUGACCAAGCUG
GCCGAGCAGUACGCCAAGGAGAACGGCACCAAGCUGCUGCUGACCAACGACC
CCCUGGAGGCCGCCCACGGCGGCAACGUGCUGAUCACCGACACCUGGAUCAG
CAUGGGCCAGGAGGAGGAGAAGAAGAAGCGCCUGCAGGCCUUCCAGGGCUAC
CAGGUGACCAUGAAGACCGCCAAGGUGGCCGCCAGCGACUGGACCUUCCUGC
ACUGCCUGCCCCGCAAGCCCGAGGAGGUGGACGACGAGGUGUUCUACAGCCC
CCGCAGCCUGGUGUUCCCCGAGGCCGAGAACCGCAAGUGGACCAUCAUGGCC
GUGAUGGUGAGCCUGCUGACCGACUACAGCCCCCAGCUGCAGAAGCCCAAGU
UCUGAGGUCUCUAGUAAUGAGCUGGAGCCUCGGUAGCCGUUCCUCCUGCCCG
CUGGGCCUCCCAACGGGCCCUCCUCCCCUCCUUGCACCGGCCCUUCCUGGUCU
UUGAAUAAAGUCUGAGUGGGCAUCUAG
>mARM1920
(SEQ ID NO: 118)
AUUAUUACAUCAAAACAAAAAGCCGCCACCAUGGGAGUAUUCAACCUGCGCA
UCCUGCUGAACAACGCCGCCUUCCGCAACGGCCACAACUUCAUGGUGCGCAA
CUUCCGCUGCGGCCAGCCCCUGCAGAACCGGGUGCAGCUGAAGGGCCGCGAC
CUGCUGACCCUGAAGAACUUCACCGGCGAGGAGAUCCGGUACAUGCUGUGGC
UGAGCGCCGACCUGAAGUUCCGCAUCAAGCAGAAGGGCGAGUACCUGCCCCU
GCUGCAGGGCAAGAGCCUGGGCAUGAUCUUCGAGAAGCGCAGCACCCGCACC
CGCCUGAGCACCGAGACAGGCUUCGCCCUGCUGGGCGGCCACCCCUGCUUCCU
GACCACCCAGGACAUCCACCUGGGCGUGAACGAGAGCCUGACCGACACCGCC
CGCGUGCUGAGCAGCAUGGCCGACGCCGUGCUGGCCCGCGUGUACAAGCAGA
GCGACCUGGACACCCUGGCCAAGGAGGCCAGCAUCCCCAUCAUCAACGGCCU
GAGCGACCUGUACCACCCCAUCCAGAUCCUGGCCGACUACCUGACCCUGCAG
GAGCACUACAGCAGCCUGAAGGGCCUGACCCUGAGCUGGAUCGGCGACGGCA
ACAACAUCCUGCACAGCAUCAUGAUGAGCGCCGCCAAGUUCGGCAUGCACCU
GCAGGCCGCCACCCCCAAGGGCUACGAGCCCGACGCCAGCGUGACCAAGCUG
GCCGAGCAGUACGCCAAGGAGAACGGCACCAAGCUGCUGCUGACCAACGACC
CCCUGGAGGCCGCCCACGGCGGCAACGUGCUGAUCACCGACACCUGGAUCAG
CAUGGGCCAGGAGGAGGAGAAGAAGAAGCGCCUGCAGGCCUUCCAGGGCUAC
CAGGUGACCAUGAAGACCGCCAAGGUGGCCGCCAGCGACUGGACCUUCCUGC
ACUGCCUGCCCCGCAAGCCCGAGGAGGUGGACGACGAGGUGUUCUACAGCCC
CCGCAGCCUGGUGUUCCCCGAGGCCGAGAACCGCAAGUGGACCAUCAUGGCC
GUGAUGGUGAGCCUGCUGACCGACUACAGCCCCCAGCUGCAGAAGCCCAAGU
UCUGAGGUCUCUAGUAAUGAGCUGGAGCCUCGGUAGCCGUUCCUCCUGCCCG
CUGGGCCUCCCAACGGGCCCUCCUCCCCUCCUUGCACCGGCCCUUCCUGGUCU
UUGAAUAAAGUCUGAGUGGGCAUCUAG
>mARM1921
(SEQ ID NO: 119)
AUUAUUACAUCAAAACAAAAAGCCGCCACCAUGCUGGUAUUCAACCUGCGCA
UCCUGCUGAACAACGCCGCCUUCCGCAACGGCCACAACUUCAUGGUGCGCAA
CUUCCGCUGCGGCCAGCCCCUGCAGAACCGGGUGCAGCUGAAGGGCCGCGAC
CUGCUGACCCUGAAGAACUUCACCGGCGAGGAGAUCCGGUACAUGCUGUGGC
UGAGCGCCGACCUGAAGUUCCGCAUCAAGCAGAAGGGCGAGUACCUGCCCCU
GCUGCAGGGCAAGAGCCUGGGCAUGAUCUUCGAGAAGCGCAGCACCCGCACC
CGCCUGAGCACCGAGACAGGCUUCGCCCUGCUGGGCGGCCACCCCUGCUUCCU
GACCACCCAGGACAUCCACCUGGGCGUGAACGAGAGCCUGACCGACACCGCC
CGCGUGCUGAGCAGCAUGGCCGACGCCGUGCUGGCCCGCGUGUACAAGCAGA
GCGACCUGGACACCCUGGCCAAGGAGGCCAGCAUCCCCAUCAUCAACGGCCU
GAGCGACCUGUACCACCCCAUCCAGAUCCUGGCCGACUACCUGACCCUGCAG
GAGCACUACAGCAGCCUGAAGGGCCUGACCCUGAGCUGGAUCGGCGACGGCA
ACAACAUCCUGCACAGCAUCAUGAUGAGCGCCGCCAAGUUCGGCAUGCACCU
GCAGGCCGCCACCCCCAAGGGCUACGAGCCCGACGCCAGCGUGACCAAGCUG
GCCGAGCAGUACGCCAAGGAGAACGGCACCAAGCUGCUGCUGACCAACGACC
CCCUGGAGGCCGCCCACGGCGGCAACGUGCUGAUCACCGACACCUGGAUCAG
CAUGGGCCAGGAGGAGGAGAAGAAGAAGCGCCUGCAGGCCUUCCAGGGCUAC
CAGGUGACCAUGAAGACCGCCAAGGUGGCCGCCAGCGACUGGACCUUCCUGC
ACUGCCUGCCCCGCAAGCCCGAGGAGGUGGACGACGAGGUGUUCUACAGCCC
CCGCAGCCUGGUGUUCCCCGAGGCCGAGAACCGCAAGUGGACCAUCAUGGCC
GUGAUGGUGAGCCUGCUGACCGACUACAGCCCCCAGCUGCAGAAGCCCAAGU
UCUGAGGUCUCUAGUAAUGAGCUGGAGCCUCGGUAGCCGUUCCUCCUGCCCG
CUGGGCCUCCCAACGGGCCCUCCUCCCCUCCUUGCACCGGCCCUUCCUGGUCU
UUGAAUAAAGUCUGAGUGGGCAUCUAG
>mARM1925
(SEQ ID NO: 120)
UCAACACAACAUAUACAAAACAAACGAAUCUCAAGCAAUCAAGCAUUCUACU
UCUAUUGCAGCAAUUUAAAUCAUUUCUUUUAAAGCAAAAGCAAUUUUCUGA
AAAUUUUCACCAUUUACGAACGAUAGCCACCAUGUUGUUCAACUUGAGGAUC
UUGUUGAACAACGCCGCCUUCAGGAACGGACACAACUUCAUGGUAAGGAACU
UCAGGUGCGGACAGCCCUUGCAGAACAAAGUACAGUUGAAAGGAAGGGACUU
GUUGACAUUGAAAAACUUCACAGGAGAAGAAAUCAAAUACAUGUUGUGGUU
GUCGGCCGACUUGAAAUUCAGGAUCAAACAGAAAGGAGAAUACUUGCCCUUG
UUGCAGGGAAAAUCGUUGGGAAUGAUCUUCGAAAAAAGGUCGACAAGGACA
AGGUUGUCGACAGAAACAGGAUUCGCCUUGUUGGGAGGACACCCCUGCUUCU
UGACAACACAGGACAUCCACUUGGGAGUAAACGAAUCGUUGACAGACACAGC
CAGGGUAUUGUCGUCGAUGGCCGACGCCGUAUUGGCCAGGGUAUACAAACAG
UCGGACUUGGACACAUUGGCCAAAGAAGCCUCGAUCCCCAUCAUCAACGGAU
UGUCGGACUUGUACCACCCCAUCCAGAUCUUGGCCGACUACUUGACAUUGCA
GGAACACUACUCGUCGUUGAAAGGAUUGACAUUGUCGUGGAUCGGAGACGG
AAACAACAUCUUGCACUCGAUCAUGAUGUCGGCCGCCAAAUUCGGAAUGCAC
UUGCAGGCCGCCACACCCAAAGGAUACGAACCCGACGCCUCGGUAACAAAAU
UGGCCGAACAGUACGCCAAAGAAAACGGAACAAAAUUGUUGUUGACAAACGA
CCCCUUGGAAGCCGCCCACGGAGGAAACGUAUUGAUCACAGACACAUGGAUC
UCGAUGGGACAGGAAGAAGAAAAAAAAAAAAGGUUGCAGGCCUUCCAGGGA
UACCAGGUAACAAUGAAAACAGCCAAAGUAGCCGCCUCGGACUGGACAUUCU
UGCACUGCUUGCCCAGGAAACCCGAAGAAGUAGACGACGAAGUAUUCUACUC
GCCCAGGUCGUUGGUAUUCCCCGAAGCCGAAAACAGGAAAUGGACAAUCAUG
GCCGUAAUGGUAUCGUUGUUGACAGACUACUCGCCCCAGUUGCAGAAACCCA
AAUUCUGAAUAGUGAACUCGAGCUAGUGACUGACUAGGAUCUGGUUACCACU
AAACCAGCCUCAAGAACACCCGAAUGGAGUCUCUAAGCUACAUAAUACCAAC
UUACACUUACAAAAUGUUGUCCCCCAAAAUGUAGCCAUUCGUAUCUGCUCCU
AAUAAAAAGAAAGUUUCUUCACAUUCUAG
>mARM1926
(SEQ ID NO: 121)
AUUAUUACAUCAAAACAAAAAGCCGCCACCAUGCUGUUCAACCUGCGCAUCC
UGCUGAACAACGCCGCCUUCCGCAACGGCCACAACUUCAUGGUGCGCAACUU
CCGCUGCGGCCAGCCCCUGCAGGGCAAGGUGCAGCUGAAGGGCCGCGACCUG
CUGACCCUGAAGAACUUCACCGGCGAGGAGAUCAAGUACAUGCUGUGGCUGA
GCGCCGACCUGAAGUUCCGCAUCAAGCAGAAGGGCGAGUACCUGCCCCUGCU
GCAGGGCAAGAGCCUGGGCAUGAUCUUCGAGAAGCGCAGCACCCGCACCCGC
CUGAGCACCGAGACAGGCUUCGCCCUGCUGGGCGGCCACCCCUGCUUCCUGA
CCACCCAGGACAUCCACCUGGGCGUGAACGAGAGCCUGACCGACACCGCCCGC
GUGCUGAGCAGCAUGGCCGACGCCGUGCUGGCCCGCGUGUACAAGCAGAGCG
ACCUGGACACCCUGGCCAAGGAGGCCAGCAUCCCCAUCAUCAACGGCCUGAG
CGACCUGUACCACCCCAUCCAGAUCCUGGCCGACUACCUGACCCUGCAGGAGC
ACUACAGCAGCCUGAAGGGCCUGACCCUGAGCUGGAUCGGCGACGGCAACAA
CAUCCUGCACAGCAUCAUGAUGAGCGCCGCCAAGUUCGGCAUGCACCUGCAG
GCCGCCACCCCCAAGGGCUACGAGCCCGACGCCAGCGUGACCAAGCUGGCCGA
GCAGUACGCCAAGGAGAACGGCACCAAGCUGCUGCUGACCAACGACCCCCUG
GAGGCCGCCCACGGCGGCAACGUGCUGAUCACCGACACCUGGAUCAGCAUGG
GCCAGGAGGAGGAGAAGAAGAAGCGCCUGCAGGCCUUCCAGGGCUACCAGGU
GACCAUGAAGACCGCCAAGGUGGCCGCCAGCGACUGGACCUUCCUGCACUGC
CUGCCCCGCAAGCCCGAGGAGGUGGACGACGAGGUGUUCUACAGCCCCCGCA
GCCUGGUGUUCCCCGAGGCCGAGAACCGCAAGUGGACCAUCAUGGCCGUGAU
GGUGAGCCUGCUGACCGACUACAGCCCCCAGCUGCAGAAGCCCAAGUUCUGA
GGUCUCUAGUAAUGAGCUGGAGCCUCGGUAGCCGUUCCUCCUGCCCGCUGGG
CCUCCCAACGGGCCCUCCUCCCCUCCUUGCACCGGCCCUUCCUGGUCUUUGAA
UAAAGUCUGAGUGGGCAUCUAG
>mARM1927
(SEQ ID NO: 122)
AUUAUUACAUCAAAACAAAAAGCCGCCACCAUGCUGUUCAACCUGCGCAUCC
UGCUGAACAACGCCGCCUUCCGCAACGGCCACAACUUCAUGGUGCGCAACUU
CCGCUGCGGCCAGCCCCUGCAGGGCCGGGUGCAGCUGAAGGGCCGCGACCUG
CUGACCCUGAAGAACUUCACCGGCGAGGAGAUCAAGUACAUGCUGUGGCUGA
GCGCCGACCUGAAGUUCCGCAUCAAGCAGAAGGGCGAGUACCUGCCCCUGCU
GCAGGGCAAGAGCCUGGGCAUGAUCUUCGAGAAGCGCAGCACCCGCACCCGC
CUGAGCACCGAGACAGGCUUCGCCCUGCUGGGCGGCCACCCCUGCUUCCUGA
CCACCCAGGACAUCCACCUGGGCGUGAACGAGAGCCUGACCGACACCGCCCGC
GUGCUGAGCAGCAUGGCCGACGCCGUGCUGGCCCGCGUGUACAAGCAGAGCG
ACCUGGACACCCUGGCCAAGGAGGCCAGCAUCCCCAUCAUCAACGGCCUGAG
CGACCUGUACCACCCCAUCCAGAUCCUGGCCGACUACCUGACCCUGCAGGAGC
ACUACAGCAGCCUGAAGGGCCUGACCCUGAGCUGGAUCGGCGACGGCAACAA
CAUCCUGCACAGCAUCAUGAUGAGCGCCGCCAAGUUCGGCAUGCACCUGCAG
GCCGCCACCCCCAAGGGCUACGAGCCCGACGCCAGCGUGACCAAGCUGGCCGA
GCAGUACGCCAAGGAGAACGGCACCAAGCUGCUGCUGACCAACGACCCCCUG
GAGGCCGCCCACGGCGGCAACGUGCUGAUCACCGACACCUGGAUCAGCAUGG
GCCAGGAGGAGGAGAAGAAGAAGCGCCUGCAGGCCUUCCAGGGCUACCAGGU
GACCAUGAAGACCGCCAAGGUGGCCGCCAGCGACUGGACCUUCCUGCACUGC
CUGCCCCGCAAGCCCGAGGAGGUGGACGACGAGGUGUUCUACAGCCCCCGCA
GCCUGGUGUUCCCCGAGGCCGAGAACCGCAAGUGGACCAUCAUGGCCGUGAU
GGUGAGCCUGCUGACCGACUACAGCCCCCAGCUGCAGAAGCCCAAGUUCUGA
GGUCUCUAGUAAUGAGCUGGAGCCUCGGUAGCCGUUCCUCCUGCCCGCUGGG
CCUCCCAACGGGCCCUCCUCCCCUCCUUGCACCGGCCCUUCCUGGUCUUUGAA
UAAAGUCUGAGUGGGCAUCUAG
>mARM1928
(SEQ ID NO: 123)
AUUAUUACAUCAAAACAAAAAGCCGCCACCAUGCUGUUCAACCUGCGCAUCC
UGCUGAACAACGCCGCCUUCCGCAACGGCCACAACUUCAUGGUGCGCAACUU
CCGCUGCGGCCAGCCCCUGCAGGGCCGGGUGCAGCUGAAGGGCCGCGACCUG
CUGACCCUGAAGAACUUCACCGGCGAGGAGAUCCGGUACAUGCUGUGGCUGA
GCGCCGACCUGAAGUUCCGCAUCAAGCAGAAGGGCGAGUACCUGCCCCUGCU
GCAGGGCAAGAGCCUGGGCAUGAUCUUCGAGAAGCGCAGCACCCGCACCCGC
CUGAGCACCGAGACAGGCUUCGCCCUGCUGGGCGGCCACCCCUGCUUCCUGA
CCACCCAGGACAUCCACCUGGGCGUGAACGAGAGCCUGACCGACACCGCCCGC
GUGCUGAGCAGCAUGGCCGACGCCGUGCUGGCCCGCGUGUACAAGCAGAGCG
ACCUGGACACCCUGGCCAAGGAGGCCAGCAUCCCCAUCAUCAACGGCCUGAG
CGACCUGUACCACCCCAUCCAGAUCCUGGCCGACUACCUGACCCUGCAGGAGC
ACUACAGCAGCCUGAAGGGCCUGACCCUGAGCUGGAUCGGCGACGGCAACAA
CAUCCUGCACAGCAUCAUGAUGAGCGCCGCCAAGUUCGGCAUGCACCUGCAG
GCCGCCACCCCCAAGGGCUACGAGCCCGACGCCAGCGUGACCAAGCUGGCCGA
GCAGUACGCCAAGGAGAACGGCACCAAGCUGCUGCUGACCAACGACCCCCUG
GAGGCCGCCCACGGCGGCAACGUGCUGAUCACCGACACCUGGAUCAGCAUGG
GCCAGGAGGAGGAGAAGAAGAAGCGCCUGCAGGCCUUCCAGGGCUACCAGGU
GACCAUGAAGACCGCCAAGGUGGCCGCCAGCGACUGGACCUUCCUGCACUGC
CUGCCCCGCAAGCCCGAGGAGGUGGACGACGAGGUGUUCUACAGCCCCCGCA
GCCUGGUGUUCCCCGAGGCCGAGAACCGCAAGUGGACCAUCAUGGCCGUGAU
GGUGAGCCUGCUGACCGACUACAGCCCCCAGCUGCAGAAGCCCAAGUUCUGA
GGUCUCUAGUAAUGAGCUGGAGCCUCGGUAGCCGUUCCUCCUGCCCGCUGGG
CCUCCCAACGGGCCCUCCUCCCCUCCUUGCACCGGCCCUUCCUGGUCUUUGAA
UAAAGUCUGAGUGGGCAUCUAG
>mARM1929
(SEQ ID NO: 124)
GGCAGAAAAAUUUGCUACAUUGUUUCACAAACUUCAAAUAUUAUUCAUUUA
UUUAGAUCUAUUAUUACAUCAAAACAAAAAGCCGCCACCAUGCUGUUCAACC
UGCGCAUCCUGCUGAACAACGCCGCCUUCCGCAACGGCCACAACUUCAUGGU
GCGCAACUUCCGCUGCGGCCAGCCCCUGCAGGGCAAGGUGCAGCUGAAGGGC
CGCGACCUGCUGACCCUGAAGAACUUCACCGGCGAGGAGAUCAAGUACAUGC
UGUGGCUGAGCGCCGACCUGAAGUUCCGCAUCAAGCAGAAGGGCGAGUACCU
GCCCCUGCUGCAGGGCAAGAGCCUGGGCAUGAUCUUCGAGAAGCGCAGCACC
CGCACCCGCCUGAGCACCGAGACAGGCUUCGCCCUGCUGGGCGGCCACCCCUG
CUUCCUGACCACCCAGGACAUCCACCUGGGCGUGAACGAGAGCCUGACCGAC
ACCGCCCGCGUGCUGAGCAGCAUGGCCGACGCCGUGCUGGCCCGCGUGUACA
AGCAGAGCGACCUGGACACCCUGGCCAAGGAGGCCAGCAUCCCCAUCAUCAA
CGGCCUGAGCGACCUGUACCACCCCAUCCAGAUCCUGGCCGACUACCUGACCC
UGCAGGAGCACUACAGCAGCCUGAAGGGCCUGACCCUGAGCUGGAUCGGCGA
CGGCAACAACAUCCUGCACAGCAUCAUGAUGAGCGCCGCCAAGUUCGGCAUG
CACCUGCAGGCCGCCACCCCCAAGGGCUACGAGCCCGACGCCAGCGUGACCAA
GCUGGCCGAGCAGUACGCCAAGGAGAACGGCACCAAGCUGCUGCUGACCAAC
GACCCCCUGGAGGCCGCCCACGGCGGCAACGUGCUGAUCACCGACACCUGGA
UCAGCAUGGGCCAGGAGGAGGAGAAGAAGAAGCGCCUGCAGGCCUUCCAGGG
CUACCAGGUGACCAUGAAGACCGCCAAGGUGGCCGCCAGCGACUGGACCUUC
CUGCACUGCCUGCCCCGCAAGCCCGAGGAGGUGGACGACGAGGUGUUCUACA
GCCCCCGCAGCCUGGUGUUCCCCGAGGCCGAGAACCGCAAGUGGACCAUCAU
GGCCGUGAUGGUGAGCCUGCUGACCGACUACAGCCCCCAGCUGCAGAAGCCC
AAGUUCUGAGGUCUCUAGUAAUGAGCUGGAGCCUCGGUAGCCGUUCCUCCUG
CCCGCUGGGCCUCCCAACGGGCCCUCCUCCCCUCCUUGCACCGGCCCUUCCUG
GUCUUUGAAUAAAGUCUGAGUGGGCAUCUAG
AT1G67090>
(SEQ ID NO: 125)
CACAAAGAGUAAAGAAGAACA
AT1G35720>
(SEQ ID NO: 126)
AACACUAAAAGUAGAAGAAAA
AT5G45900>
(SEQ ID NO: 127)
CUCAGAAAGAUAAGAUCAGCC
>pARM563
(SEQ ID NO: 128)
ATGCTGTTTAATCTGAGGATCCTGTTAAACAATGCAGCTTTTAGAAATGGTCAC
AACTTCATGGTTCGAAATTTTCGGTGTGGACAACCACTACAAAATAAAGTGCAG
CTGAAGGGCCGTGACCTTCTCACTCTAAAAAACTTTACCGGAGAAGAAATTAAA
TATATGCTATGGCTATCAGCAGATCTGAAATTTAGGATAAAACAGAAAGGAGA
GTATTTGCCTTTATTGCAAGGGAAGTCCTTAGGCATGATTTTTGAGAAAAGAAG
TACTCGAACAAGATTGTCTACAGAAACAGGCTTTGCACTTCTGGGAGGACATCC
TTGTTTTCTTACCACACAAGATATTCATTTGGGTGTGAATGAAAGTCTCACGGA
CACGGCCCGTGTATTGTCTAGCATGGCAGATGCAGTATTGGCTCGAGTGTATAA
ACAATCAGATTTGGACACCCTGGCTAAAGAAGCATCCATCCCAATTATCAATGG
GCTGTCAGATTTGTACCATCCTATCCAGATCCTGGCTGATTACCTCACGCTCCAG
GAACACTATAGCTCTCTGAAAGGTCTTACCCTCAGCTGGATCGGGGATGGGAAC
AATATCCTGCACTCCATCATGATGAGCGCAGCGAAATTCGGAATGCACCTTCAG
GCAGCTACTCCAAAGGGTTATGAGCCGGATGCTAGTGTAACCAAGTTGGCAGA
GCAGTATGCCAAAGAGAATGGTACCAAGCTGTTGCTGACAAATGATCCATTGG
AAGCAGCGCATGGAGGCAATGTATTAATTACAGACACTTGGATAAGCATGGGA
CAAGAAGAGGAGAAGAAAAAGCGGCTCCAGGCTTTCCAAGGTTACCAGGTTAC
AATGAAGACTGCTAAAGTTGCTGCCTCTGACTGGACATTTTTACACTGCTTGCC
CAGAAAGCCAGAAGAAGTGGATGATGAAGTCTTTTATTCTCCTCGATCACTAGT
GTTCCCAGAGGCAGAAAACAGAAAGTGGACAATCATGGCTGTCATGGTGTCCC
TGCTGACAGATTACTCACCTCAGCTCCAGAAGCCTAAATTTTGA
>pARM564
(SEQ ID NO: 129)
ATGCTCTTTAATCTGCGCATCTTACTGAACAACGCCGCATTCCGGAACGGTCAC
AACTTCATGGTCCGCAATTTCCGCTGTGGCCAGCCGCTTCAAAACAAGGTCCAG
CTGAAGGGACGGGATCTGCTGACACTGAAGAACTTCACCGGAGAAGAGATCAA
GTACATGCTGTGGCTCAGCGCAGACTTGAAGTTCCGGATCAAGCAGAAGGGAG
AATACTTGCCCCTGCTGCAAGGAAAGTCGCTGGGAATGATTTTTGAGAAGCGGT
CAACTCGCACCAGACTCTCCACCGAAACTGGTTTCGCACTGCTTGGCGGGCACC
CTTGCTTCCTGACGACTCAGGACATCCACCTCGGCGTGAACGAATCGCTAACCG
ATACCGCCAGAGTGCTTTCTTCCATGGCCGACGCGGTGCTGGCCAGGGTGTACA
AGCAGTCCGACCTCGATACCTTGGCAAAGGAGGCTTCCATTCCCATCATCAACG
GCCTGAGCGACCTGTACCACCCAATCCAAATCCTGGCTGACTACCTGACCCTGC
AAGAGCACTACAGCAGCCTGAAGGGTCTGACCCTGTCATGGATTGGCGATGGA
AACAATATTCTGCACTCCATCATGATGTCCGCCGCGAAGTTCGGAATGCATCTG
CAAGCCGCCACTCCAAAAGGATACGAACCGGATGCGTCCGTGACCAAGTTGGC
GGAACAGTACGCGAAGGAGAACGGAACCAAGCTTCTGCTGACTAACGACCCCC
TCGAGGCTGCGCATGGGGGCAACGTGCTGATTACCGACACCTGGATCTCCATGG
GGCAGGAGGAAGAGAAGAAGAAGAGACTGCAGGCATTCCAGGGGTACCAGGT
CACCATGAAAACCGCAAAAGTGGCAGCTTCGGACTGGACTTTCCTGCATTGCCT
GCCGAGGAAGCCGGAGGAAGTCGACGACGAAGTGTTCTACTCGCCTCGGTCCC
TGGTGTTCCCCGAGGCCGAAAACCGGAAGTGGACCATCATGGCCGTGATGGTG
TCCTTGCTGACTGACTATAGCCCGCAGCTGCAGAAGCCTAAGTTCTAG
>pARM565
(SEQ ID NO: 130)
ATGCTGTTTAACCTACGTATTTTGCTCAACAATGCAGCCTTTAGAAACGGACAT
AACTTTATGGTTCGAAACTTTCGCTGCGGGCAGCCACTGCAGAACAAGGTCCAG
CTGAAAGGGAGAGATTTGCTCACGCTGAAGAACTTTACTGGCGAAGAAATCAA
GTATATGCTGTGGTTGTCCGCGGACCTCAAGTTTCGGATTAAGCAGAAAGGGGA
GTATCTGCCACTGCTGCAAGGAAAGAGCCTCGGCATGATCTTCGAGAAGCGGA
GCACTCGGACCAGGCTGAGTACCGAAACTGGCTTCGCATTGTTGGGTGGACATC
CATGTTTTCTGACAACGCAGGACATTCATCTGGGCGTGAACGAGAGTCTGACGG
ACACAGCTCGCGTTCTGTCCTCTATGGCTGATGCGGTGTTGGCCCGGGTCTATA
AGCAGTCCGATTTGGACACCTTGGCTAAGGAAGCTAGCATACCGATTATCAATG
GGCTGTCCGACCTGTATCACCCTATTCAAATCCTGGCCGACTACCTCACACTGC
AAGAACACTATAGCTCATTGAAGGGACTGACCCTGAGCTGGATAGGGGACGGA
AACAACATCCTACATAGCATTATGATGTCCGCTGCCAAGTTTGGCATGCATCTT
CAAGCCGCCACGCCAAAGGGTTATGAGCCCGACGCGTCAGTGACAAAGCTGGC
CGAGCAGTACGCTAAGGAGAATGGTACCAAATTACTGCTGACTAATGATCCACT
GGAGGCTGCACATGGCGGCAATGTACTGATCACCGACACATGGATCTCGATGG
GCCAGGAGGAAGAAAAGAAGAAGAGGCTTCAGGCCTTCCAAGGCTACCAGGTC
ACCATGAAAACAGCTAAGGTTGCAGCATCTGATTGGACCTTTCTGCACTGTCTG
CCAAGGAAGCCCGAAGAGGTGGACGATGAAGTATTCTATAGCCCACGGAGTTT
GGTGTTCCCTGAGGCTGAAAATAGGAAGTGGACAATTATGGCCGTAATGGTGTC
CCTGTTAACCGACTACTCTCCGCAACTGCAGAAACCTAAGTTTTAG
>pARM566
(SEQ ID NO: 131)
ATGCTGTTTAACTTAAGGATCCTGCTGAACAACGCCGCTTTTCGTAACGGTCAT
AACTTTATGGTCCGGAACTTTAGATGTGGCCAGCCGCTGCAGAACAAGGTTCAG
CTGAAGGGGAGGGATCTGCTGACCTTGAAGAACTTTACCGGCGAAGAGATCAA
GTACATGTTGTGGCTGAGCGCCGATCTGAAGTTTAGGATTAAGCAGAAGGGGG
AGTATTTGCCACTGCTGCAAGGAAAATCCTTGGGGATGATCTTCGAGAAGCGCT
CCACTAGAACCCGGCTAAGCACAGAAACCGGCTTCGCACTTCTGGGTGGACATC
CCTGTTTTCTGACGACGCAGGATATACACCTGGGCGTGAATGAGAGTCTGACGG
ACACAGCTAGGGTGTTGAGCAGCATGGCCGATGCAGTACTGGCCCGCGTTTATA
AGCAGAGCGACTTGGACACACTGGCCAAGGAAGCGTCAATTCCGATTATCAAT
GGGCTGTCAGACCTGTATCATCCCATTCAAATCTTGGCTGACTATCTGACCCTGC
AAGAACATTACAGCTCCCTGAAGGGCCTCACGTTGTCCTGGATTGGCGACGGAA
ACAACATTCTGCATTCGATCATGATGAGCGCTGCTAAGTTTGGCATGCACCTCC
AAGCCGCTACACCTAAGGGATATGAGCCTGATGCCAGCGTAACCAAGCTGGCC
GAACAGTACGCGAAGGAGAATGGCACGAAACTGCTGTTGACAAATGACCCACT
GGAGGCAGCTCACGGTGGCAACGTGCTGATCACCGACACGTGGATATCTATGG
GACAGGAAGAAGAGAAGAAGAAGCGGCTGCAGGCATTCCAAGGGTATCAGGT
CACCATGAAAACGGCCAAGGTTGCTGCATCCGACTGGACATTTCTGCATTGCTT
GCCCCGCAAACCAGAAGAAGTAGACGACGAAGTCTTTTATTCCCCACGGTCGCT
GGTGTTCCCCGAGGCGGAGAATCGAAAGTGGACGATTATGGCCGTGATGGTGT
CCCTGCTGACTGATTACTCTCCCCAACTGCAAAAGCCTAAGTTTTAG
>pARM567
(SEQ ID NO: 132)
ATGCTTTTCAACCTGAGGATCCTCCTGAACAACGCCGCCTTTCGCAATGGTCAC
AACTTTATGGTCCGGAACTTCAGATGCGGCCAGCCGCTGCAGAACAAGGTCCA
GCTGAAGGGACGGGATCTGCTGACTCTGAAGAACTTCACCGGAGAAGAGATCA
AGTACATGCTGTGGCTGTCGGCCGACCTGAAGTTCAGGATCAAGCAGAAGGGA
GAATACCTCCCGCTGCTGCAAGGAAAGTCCCTGGGCATGATTTTCGAGAAGCGC
TCGACCAGAACTCGGTTGTCCACCGAAACCGGGTTTGCGCTGCTGGGCGGACAT
CCTTGCTTCCTGACGACTCAGGATATTCACCTGGGAGTGAACGAGTCGCTGACC
GACACCGCCAGAGTGCTGAGCTCGATGGCCGACGCCGTGTTGGCACGCGTGTA
CAAGCAGTCCGATCTGGATACCCTGGCCAAAGAAGCTTCCATCCCGATCATTAA
CGGGCTGAGCGACCTCTACCACCCCATTCAAATCCTGGCCGACTACCTGACTCT
GCAAGAACACTACAGCTCGCTGAAGGGGTTGACTCTGTCCTGGATCGGCGACG
GAAACAACATCCTGCACTCCATCATGATGTCGGCCGCAAAGTTCGGCATGCATT
TGCAAGCCGCCACCCCAAAGGGCTACGAACCAGACGCGAGCGTCACCAAGCTG
GCCGAACAGTACGCGAAGGAAAATGGTACTAAGCTGCTGCTGACCAACGACCC
ATTGGAAGCTGCCCATGGTGGAAACGTGCTGATCACCGACACCTGGATCTCGAT
GGGCCAGGAAGAGGAGAAGAAGAAGCGGCTGCAGGCGTTCCAGGGGTATCAG
GTCACCATGAAAACAGCCAAAGTGGCAGCGTCAGACTGGACCTTCCTCCACTGT
CTGCCTCGCAAGCCAGAGGAGGTGGACGACGAGGTGTTCTACTCCCCTCGGTCC
CTCGTGTTCCCTGAGGCTGAGAACCGGAAGTGGACCATTATGGCCGTGATGGTG
TCACTCCTGACTGATTACTCCCCGCAACTGCAGAAGCCCAAGTTCTAG
>pARM568
(SEQ ID NO: 133)
ATGCTGTTTAACCTGAGGATCCTATTGAACAATGCTGCTTTTCGTAATGGCCATA
ACTTTATGGTTCGGAACTTTAGATGCGGGCAGCCACTGCAGAACAAGGTCCAGT
TGAAAGGCCGCGATCTGTTGACATTGAAGAACTTTACCGGCGAAGAGATTAAG
TATATGCTGTGGCTGTCTGCTGACCTCAAGTTTCGAATCAAGCAGAAGGGCGAA
TATCTCCCCCTGCTGCAAGGAAAGTCTCTCGGCATGATCTTTGAGAAGCGGAGT
ACCCGAACACGGCTGAGCACCGAAACGGGCTTCGCACTGCTGGGGGGCCATCC
CTGTTTTCTGACAACGCAGGACATCCACTTGGGGGTTAACGAATCATTGACTGA
TACCGCCCGCGTACTGTCATCCATGGCCGACGCTGTGCTGGCTAGGGTGTACAA
GCAGTCAGATCTGGATACACTGGCCAAGGAAGCTAGCATACCAATCATCAATG
GACTGAGTGACCTTTATCACCCGATTCAAATACTAGCCGATTATCTGACCCTGC
AAGAGCATTACTCCTCGCTGAAAGGCCTCACGCTGTCCTGGATCGGCGACGGCA
ACAACATTCTGCATAGTATTATGATGTCTGCTGCCAAATTCGGCATGCATCTGC
AAGCTGCTACGCCGAAGGGTTATGAACCCGACGCGTCAGTTACGAAGCTCGCT
GAGCAGTATGCAAAGGAGAATGGCACAAAGCTGTTGCTTACCAACGATCCCCT
GGAAGCTGCTCATGGCGGCAATGTGCTGATTACTGACACCTGGATTTCAATGGG
CCAGGAGGAGGAGAAGAAGAAGAGGTTACAGGCTTTTCAAGGTTACCAAGTCA
CGATGAAAACCGCTAAGGTCGCAGCCAGCGACTGGACATTCCTGCACTGTCTGC
CAAGAAAGCCGGAAGAAGTGGACGACGAGGTGTTCTATTCCCCGCGGTCTTTG
GTGTTTCCGGAGGCCGAAAACAGGAAATGGACCATTATGGCCGTGATGGTATC
GTTGCTGACGGACTACAGCCCTCAGTTGCAAAAGCCCAAGTTCTAG
>pARM569
(SEQ ID NO: 134)
ATGCTCTTTAACCTCCGCATCCTCCTCAACAACGCCGCCTTCCGGAATGGGCAT
AACTTCATGGTCCGGAACTTCAGATGCGGCCAGCCCCTGCAAAACAAGGTCCA
GTTGAAGGGACGGGACCTCCTTACGCTGAAGAACTTTACCGGAGAAGAGATTA
AGTACATGCTGTGGTTGTCCGCTGACCTCAAGTTCCGCATTAAGCAGAAGGGAG
AATATCTGCCGCTGCTGCAAGGAAAGAGCCTGGGCATGATCTTCGAAAAGCGC
TCCACTAGAACCCGGCTGTCGACTGAGACTGGATTCGCCTTGCTCGGTGGACAC
CCGTGCTTCCTGACGACCCAGGACATCCACCTGGGAGTGAACGAGTCACTTACG
GATACCGCGAGGGTGCTGTCCTCAATGGCCGACGCAGTGCTCGCGCGCGTGTAC
AAGCAGTCAGATCTGGATACCCTGGCCAAGGAAGCCAGCATTCCCATCATCAA
CGGACTGAGCGACCTTTACCACCCAATCCAGATCCTCGCCGACTACTTAACCCT
GCAAGAGCACTACAGCTCCCTGAAGGGACTGACTCTGTCCTGGATCGGGGATG
GAAACAACATCCTGCACTCCATCATGATGTCTGCCGCTAAGTTTGGGATGCATC
TGCAAGCCGCAACCCCTAAGGGATACGAGCCCGACGCCTCGGTGACCAAACTT
GCGGAACAGTACGCCAAGGAAAACGGTACCAAGCTGCTGCTGACCAACGACCC
TCTGGAAGCGGCCCACGGAGGAAATGTGCTGATTACCGACACCTGGATTTCGAT
GGGCCAGGAGGAGGAGAAGAAGAAGAGACTGCAGGCGTTCCAGGGATATCAG
GTCACCATGAAAACCGCCAAGGTCGCCGCCAGCGACTGGACCTTCCTGCACTGT
CTCCCTCGGAAACCGGAAGAAGTGGATGACGAGGTGTTCTACTCCCCGCGCTCG
CTGGTGTTCCCGGAGGCTGAAAACAGGAAGTGGACAATCATGGCCGTGATGGT
GTCCCTGTTGACCGACTACTCCCCACAACTGCAGAAGCCCAAGTTCTAG
>pARM570
(SEQ ID NO: 135)
ATGCTTTTCAATCTGCGCATCCTCCTGAACAACGCCGCCTTCCGCAATGGACAC
AACTTTATGGTCCGCAACTTCCGCTGTGGGCAGCCGCTGCAGAACAAGGTCCAG
CTCAAGGGGAGAGATCTCCTGACCCTGAAGAACTTCACTGGAGAGGAGATCAA
GTACATGCTGTGGCTGTCCGCCGACCTGAAATTTCGGATTAAGCAGAAGGGCGA
ATACCTCCCACTGCTGCAAGGAAAGTCTTTGGGCATGATCTTCGAAAAGAGAAG
CACCCGGACCCGGTTGAGCACCGAAACTGGGTTCGCGCTCCTCGGTGGACACCC
GTGCTTCCTGACCACCCAAGATATTCATCTGGGTGTCAACGAAAGCCTGACCGA
CACCGCCAGGGTGCTGTCATCCATGGCTGACGCAGTGCTCGCCCGGGTGTACAA
GCAGTCAGACCTGGACACCCTCGCCAAGGAAGCTTCGATCCCTATCATCAACGG
ACTTTCCGACCTGTACCACCCCATCCAAATTCTGGCCGACTACCTGACTCTGCA
AGAACACTATAGCTCGCTGAAAGGACTTACTCTGTCCTGGATCGGGGACGGCA
ACAACATTCTCCATTCCATCATGATGTCCGCTGCCAAGTTCGGAATGCACCTTC
AAGCAGCGACTCCCAAGGGATACGAACCTGATGCCTCCGTGACTAAGCTGGCA
GAGCAGTACGCCAAGGAGAACGGTACAAAGCTGCTGCTCACGAACGACCCCCT
GGAGGCGGCCCACGGCGGAAACGTGCTGATTACCGATACCTGGATCTCAATGG
GCCAGGAAGAGGAGAAGAAGAAGCGGCTCCAGGCGTTTCAAGGCTACCAGGTC
ACCATGAAAACCGCGAAGGTCGCCGCCTCCGACTGGACTTTCTTGCACTGCCTG
CCGCGGAAGCCCGAGGAAGTGGATGACGAAGTGTTCTACTCGCCGAGATCGTT
GGTGTTCCCTGAGGCCGAAAACAGGAAGTGGACCATCATGGCCGTGATGGTGT
CCCTGCTGACTGATTACAGCCCACAGCTGCAGAAGCCTAAGTTCTAG
>pARM571
(SEQ ID NO: 136)
ATGCTTTTCAATCTCCGCATCCTCCTTAACAACGCCGCGTTTAGAAACGGCCAC
AACTTCATGGTCCGGAACTTCAGATGTGGCCAGCCGCTTCAAAACAAGGTCCAG
CTGAAGGGCCGGGATCTTCTGACCCTGAAGAACTTTACTGGCGAAGAGATCAA
GTACATGCTCTGGCTCTCCGCGGACTTGAAGTTCCGCATTAAGCAGAAGGGGGA
ATACCTTCCGCTGCTTCAAGGAAAGAGCCTCGGCATGATCTTTGAGAAGCGCTC
AACCAGGACCCGCCTTTCTACTGAAACTGGGTTCGCGCTGCTCGGTGGCCACCC
CTGCTTCCTGACGACCCAGGACATCCACCTCGGAGTGAACGAATCCCTCACCGA
TACCGCCCGGGTGTTATCGAGCATGGCAGATGCCGTGCTGGCCAGGGTGTACAA
ACAGTCCGATCTGGACACTCTGGCCAAGGAGGCGTCAATTCCTATTATCAACGG
CCTTAGTGACCTCTACCATCCGATTCAGATCCTGGCCGATTACCTCACCCTGCAA
GAACACTACAGCTCCCTGAAGGGTCTGACATTGTCCTGGATCGGCGACGGCAAC
AACATTCTCCATTCCATCATGATGTCCGCCGCAAAATTCGGCATGCATCTTCAA
GCCGCCACGCCGAAGGGTTACGAGCCCGACGCTTCCGTGACTAAGCTCGCCGA
GCAGTACGCTAAGGAGAACGGAACCAAGCTTCTGCTGACTAACGACCCACTAG
AAGCAGCCCACGGGGGCAACGTGCTTATTACTGACACCTGGATCTCCATGGGCC
AGGAAGAAGAGAAAAAGAAGCGGCTGCAGGCGTTCCAGGGATATCAGGTCAC
CATGAAAACCGCCAAGGTCGCTGCCTCCGACTGGACCTTCCTGCACTGCCTGCC
TCGCAAGCCTGAAGAAGTGGACGACGAGGTGTTCTACTCGCCACGGAGCCTCG
TGTTCCCCGAGGCCGAGAATAGAAAGTGGACCATCATGGCCGTGATGGTGTCA
CTGCTCACCGACTACAGCCCGCAGCTTCAGAAGCCCAAGTTCTAG
>pARM572
(SEQ ID NO: 137)
ATGCTTTTCAACCTGAGAATCCTCCTGAACAACGCCGCCTTCCGCAATGGTCAT
AACTTCATGGTCCGCAACTTTCGCTGCGGACAGCCTCTCCAAAACAAGGTCCAG
CTCAAGGGGCGCGACCTCCTCACACTGAAGAACTTCACTGGAGAAGAAATCAA
GTACATGCTGTGGCTGAGCGCCGATCTGAAGTTCCGGATCAAGCAGAAGGGAG
AGTACCTTCCTCTGCTGCAAGGGAAGTCCTTGGGAATGATTTTCGAGAAGCGGT
CCACCCGGACCAGGCTGAGCACTGAAACTGGCTTCGCCCTGCTGGGAGGCCAC
CCTTGTTTCCTGACCACTCAGGACATCCACCTGGGCGTGAACGAGTCCCTGACC
GATACTGCCAGAGTGCTGTCCTCCATGGCCGACGCCGTGCTCGCCCGGGTGTAC
AAGCAGTCAGACCTCGATACGCTGGCCAAGGAAGCCTCCATTCCCATTATCAAT
GGTCTGTCGGACCTCTACCATCCAATCCAAATCCTCGCCGACTACCTGACTCTG
CAAGAACACTACAGCTCACTCAAGGGCCTCACCCTCTCCTGGATCGGCGACGGA
AACAACATCCTTCACTCGATTATGATGTCGGCCGCGAAGTTCGGGATGCACCTC
CAAGCTGCCACTCCAAAAGGCTACGAGCCGGATGCCTCAGTGACTAAGTTGGC
GGAACAGTATGCGAAGGAGAACGGTACCAAGCTCCTGCTGACTAACGACCCGC
TGGAGGCCGCCCACGGGGGAAACGTGCTCATCACCGATACTTGGATTTCCATGG
GACAGGAGGAAGAGAAGAAGAAGCGGTTGCAGGCATTTCAGGGCTACCAGGTC
ACCATGAAAACTGCCAAAGTCGCCGCCAGCGACTGGACCTTCCTGCACTGCCTG
CCCCGCAAGCCTGAAGAAGTGGACGACGAGGTGTTCTACTCTCCCCGGTCCCTC
GTGTTCCCTGAGGCCGAAAACAGGAAGTGGACCATCATGGCTGTGATGGTGTCC
CTCCTGACCGACTACAGCCCTCAGCTCCAAAAACCCAAGTTTTAG
>pARM573
(SEQ ID NO: 138)
ATGCTTTTCAACCTGAGAATCCTCTTGAACAATGCTGCTTTTCGGAATGGCCAC
AACTTTATGGTTCGGAACTTCCGTTGCGGCCAGCCTTTACAAAACAAGGTCCAG
CTGAAGGGCCGGGATTTGCTCACACTGAAGAACTTTACTGGGGAGGAGATTAA
GTATATGCTGTGGCTGTCCGCTGACCTGAAGTTTAGGATCAAGCAGAAGGGCGA
ATATCTGCCGCTGCTGCAAGGGAAAAGTCTGGGCATGATTTTTGAAAAGCGCTC
TACCCGGACCAGACTGTCTACGGAAACAGGCTTTGCCCTGCTGGGCGGCCACCC
CTGTTTTCTGACAACGCAGGACATCCATCTGGGCGTGAACGAATCACTGACCGA
TACTGCTCGGGTACTCAGTTCTATGGCTGACGCAGTGCTGGCTAGGGTGTACAA
GCAGAGCGACTTGGACACACTGGCTAAGGAGGCCAGCATCCCCATTATCAATG
GCCTGTCTGATTTGTACCATCCCATTCAAATCCTGGCTGATTATCTGACACTACA
AGAGCATTACTCAAGTCTGAAGGGTTTGACTCTCTCCTGGATCGGCGACGGCAA
CAACATTTTACATTCCATTATGATGAGTGCTGCTAAGTTTGGCATGCATTTGCAA
GCTGCTACCCCAAAGGGCTATGAACCTGACGCTAGCGTAACCAAGTTGGCCGA
ACAGTATGCTAAAGAGAATGGCACCAAGCTGCTCCTGACGAATGACCCCCTGG
AAGCTGCTCATGGCGGAAACGTACTTATAACTGATACATGGATTAGCATGGGCC
AGGAAGAGGAGAAGAAGAAGAGACTGCAGGCCTTCCAAGGCTATCAGGTCACC
ATGAAAACTGCCAAGGTTGCAGCTAGCGACTGGACCTTCCTGCACTGTTTGCCG
AGGAAACCCGAGGAGGTGGACGATGAAGTCTTTTATTCTCCCCGCTCCTTGGTG
TTTCCCGAGGCTGAAAATCGAAAGTGGACGATAATGGCAGTGATGGTGTCCCTA
CTGACCGACTATTCTCCACAACTGCAGAAGCCTAAATTCTAG
>pARM574
(SEQ ID NO: 139)
ATGCTTTTCAATCTGAGGATCCTGCTGAACAACGCTGCTTTTCGCAACGGTCAT
AACTTTATGGTTCGCAATTTTCGTTGTGGCCAGCCGCTGCAGAACAAGGTTCAG
CTGAAGGGCAGAGATCTGCTGACTCTGAAGAACTTCACTGGGGAAGAAATCAA
GTATATGTTATGGCTGTCCGCGGATCTGAAATTTCGAATCAAGCAGAAGGGCGA
ATATCTTCCCCTGCTGCAAGGGAAATCCTTGGGCATGATTTTTGAGAAGAGGAG
CACTAGGACTAGATTGTCAACAGAAACAGGCTTTGCTTTGTTGGGCGGACATCC
CTGCTTTCTGACGACACAGGATATCCACCTCGGCGTAAACGAGTCCCTCACCGA
CACTGCTAGGGTACTGAGCAGCATGGCCGACGCTGTGCTAGCCCGGGTTTACAA
GCAGTCAGACCTGGACACCCTTGCCAAGGAAGCTTCTATTCCAATTATCAACGG
CCTGAGTGACCTGTATCACCCTATTCAAATACTCGCCGACTATTTGACGCTTCAA
GAACATTACAGCAGCCTCAAGGGCTTAACCTTGAGTTGGATAGGCGACGGCAA
CAATATCCTGCATTCCATTATGATGTCTGCCGCTAAGTTTGGCATGCATCTACAA
GCCGCAACACCCAAGGGCTATGAACCCGACGCTAGCGTGACCAAGCTGGCCGA
GCAGTATGCTAAGGAAAATGGCACAAAGCTCCTTCTTACCAACGATCCCCTGGA
GGCTGCTCACGGCGGCAACGTGCTGATTACCGATACATGGATTAGCATGGGCCA
GGAGGAGGAGAAAAAGAAGCGGCTCCAGGCTTTTCAAGGCTATCAGGTCACCA
TGAAAACTGCAAAGGTCGCTGCCTCCGACTGGACTTTCCTGCATTGTCTACCCC
GCAAGCCTGAGGAAGTGGACGATGAGGTGTTCTACTCCCCACGGAGTCTGGTGT
TCCCGGAAGCAGAGAATCGGAAGTGGACCATCATGGCTGTCATGGTGTCGCTCT
TGACTGACTATTCTCCCCAACTGCAAAAACCCAAGTTTTAG
>pARM575
(SEQ ID NO: 140)
ATGCTTTTCAATCTGAGGATCCTGCTGAACAACGCTGCTTTTCGCAACGGTCAT
AACTTTATGGTTCGCAATTTTCGTTGTGGCCAGCCGCTGCAGAACAAGGTTCAG
CTGAAGGGCAGAGATCTGCTGACTCTGAAGAACTTCACTGGGGAAGAAATCAA
GTATATGTTATGGCTGTCCGCGGATCTGAAATTTCGAATCAAGCAGAAGGGCGA
ATATCTTCCCCTGCTGCAAGGGAAATCCTTGGGCATGATTTTTGAGAAGAGGAG
CACTAGGACTAGATTGTCAACAGAAACAGGCTTTGCTTTGTTGGGCGGACATCC
CTGCTTTCTGACGACACAGGATATCCACCTCGGCGTAAACGAGTCCCTCACCGA
CACTGCTAGGGTACTGAGCAGCATGGCCGACGCTGTGCTAGCCCGGGTTTACAA
GCAGTCAGACCTGGACACCCTTGCCAAGGAAGCTTCTATTCCAATTATCAACGG
CCTGAGTGACCTGTATCACCCTATTCAAATACTCGCCGACTATTTGACGCTTCAA
GAACATTACAGCAGCCTCAAGGGCTTAACCTTGAGTTGGATAGGCGACGGCAA
CAATATCCTGCATTCCATTATGATGTCTGCCGCTAAGTTTGGCATGCATCTACAA
GCCGCAACACCCAAGGGCTATGAACCCGACGCTAGCGTGACCAAGCTGGCCGA
GCAGTATGCTAAGGAAAATGGCACAAAGCTCCTTCTTACCAACGATCCCCTGGA
GGCTGCTCACGGCGGCAACGTGCTGATTACCGATACATGGATTAGCATGGGCCA
GGAGGAGGAGAAAAAGAAGCGGCTCCAGGCTTTTCAAGGCTATCAGGTCACCA
TGAAAACTGCAAAGGTCGCTGCCTCCGACTGGACTTTCCTGCATTGTCTACCCC
GCAAGCCTGAGGAAGTGGACGATGAGGTGTTCTACTCCCCACGGAGTCTGGTGT
TCCCGGAAGCAGAGAATCGGAAGTGGACCATCATGGCTGTCATGGTGTCGCTCT
TGACTGACTATTCTCCCCAACTGCAAAAACCCAAGTTTTAG
>pARM708
(SEQ ID NO: 141)
ATGCTTTTTAATCTCCGCATCCTCCTTAACAACGCCGCGTTTAGAAACGGCCAC
AACTTCATGGTCCGGAACTTCAGATGTGGCCAGCCGCTTCAAAACAAGGTCCAG
CTGAAGGGCCGGGATCTTCTGACCCTGAAGAACTTTACTGGCGAAGAGATCAA
GTACATGCTCTGGCTCTCCGCGGACTTGAAGTTCCGCATTAAGCAGAAGGGGGA
ATACCTTCCGCTGCTTCAAGGAAAGAGCCTCGGCATGATCTTTGAGAAGCGCTC
AACCAGGACCCGCCTTTCTACTGAAACTGGGTTCGCGCTGCTCGGTGGCCACCC
CTGCTTCCTGACGACCCAGGACATCCACCTCGGAGTGAACGAATCCCTCACCGA
TACCGCCCGGGTGTTATCGAGCATGGCAGATGCCGTGCTGGCCAGGGTGTACAA
ACAGTCCGATCTGGACACTCTGGCCAAGGAGGCGTCAATTCCCATCATCAACGG
CCTGAGCGACCTGTACCACCCAATCCAAATCCTGGCTGACTACCTGACCCTGCA
AGAGCACTACAGCAGCCTGAAGGGTCTGACCCTGTCATGGATTGGCGATGGAA
ACAATATTCTGCACTCCATCATGATGTCCGCCGCGAAGTTCGGAATGCATCTGC
AAGCCGCCACGCCAAAAGGATACGAACCGGATGCGTCCGTGACGAAGTTGGCG
GAACAGTACGCGAAGGAGAACGGAACCAAGCTTCTGCTGACTAACGACCCCCT
CGAGGCTGCGCATGGGGGCAACGTGCTGATTACCGACACCTGGATCTCCATGG
GGCAGGAGGAAGAGAAGAAGAAGAGACTGCAGGCATTCCAGGGGTACCAGGT
CACCATGAAAACCGCAAAAGTGGCAGCTTCGGACTGGACTTTCCTGCATTGCCT
GCCGAGGAAGCCGGAGGAAGTCGACGACGAAGTGTTCTACTCGCCTCGGTCCC
TGGTGTTCCCCGAGGCCGAAAACCGGAAGTGGACCATCATGGCCGTGATGGTG
TCCTTGCTGACTGACTATAGCCCGCAGCTGCAGAAGCCTAAGTTCTAG
>pARM709
(SEQ ID NO: 142)
ATGCTTTTCAACCTGAGAATCCTCTTGAACAATGCTGCTTTTCGGAATGGCCAC
AACTTTATGGTTCGGAACTTCCGTTGCGGCCAGCCTTTACAAAACAAGGTCCAG
CTGAAGGGCCGGGATTTGCTCACACTGAAGAACTTTACTGGAGAAGAGATCAA
GTACATGCTGTGGCTGTCGGCCGACCTGAAGTTCAGGATCAAGCAGAAGGGAG
AATACCTTCCGCTGCTTCAAGGAAAGAGCCTCGGCATGATCTTTGAGAAGCGCT
CAACCAGGACCCGCCTTTCTACTGAAACTGGGTTCGCGCTGCTCGGTGGCCACC
CCTGCTTCCTGACGACCCAGGACATCCACCTCGGAGTGAACGAATCCCTCACCG
ATACCGCCCGGGTGTTATCGAGCATGGCAGATGCCGTGCTGGCCAGGGTGTACA
AACAGTCCGATCTCGATACCTTGGCAAAGGAGGCTTCCATTCCCATCATCAACG
GCCTGAGCGACCTGTACCACCCAATCCAAATCCTGGCTGACTACCTGACCCTGC
AAGAGCACTACAGCAGCCTGAAGGGTCTGACCCTGTCATGGATTGGCGATGGA
AACAATATTCTGCACTCCATCATGATGTCCGCCGCGAAGTTCGGAATGCATCTG
CAAGCCGCCACTCCAAAAGGATACGAACCGGATGCGTCCGTGACCAAGTTGGC
GGAACAGTACGCGAAGGAGAACGGAACCAAGCTTCTGCTGACTAACGACCCCC
TCGAGGCTGCGCATGGGGGCAACGTGCTGATTACCGACACCTGGATCTCCATGG
GGCAGGAGGAAGAGAAGAAGAAGAGACTGCAGGCATTCCAGGGGTACCAGGT
CACCATGAAAACCGCAAAAGTGGCAGCTTCGGACTGGACTTTCCTGCATTGCCT
GCCGAGGAAGCCGGAGGAAGTCGACGACGAAGTGTTCTACTCGCCTCGGTCCC
TGGTGTTCCCCGAGGCCGAAAACCGGAAGTGGACCATCATGGCCGTGATGGTG
TCCTTGCTGACTGACTATAGCCCGCAGCTGCAGAAGCCTAAGTTCTAG
>pARM710
(SEQ ID NO: 143)
ATGCTTTTCAACCTGAGAATCCTCTTGAACAATGCTGCTTTTCGGAATGGCCAC
AACTTTATGGTTCGGAACTTCCGTTGCGGCCAGCCTTTACAAAACAAGGTCCAG
CTGAAGGGCCGGGATTTGCTCACACTAAAGAACTTTACTGGAGAAGAGATCAA
GTACATGCTATGGCTGTCGGCCGACCTGAAGTTCCGTATCAAGCAGAAGGGAG
AATACCTTCCGCTGCTTCAAGGAAAGAGCCTCGGCATGATCTTTGAGAAGCGCT
CAACCAGGACCCGCCTTTCTACTGAAACTGGGTTCGCGCTGCTCGGTGGCCACC
CCTGCTTCCTGACGACCCAGGACATCCACCTCGGAGTGAACGAATCCCTCACCG
ATACCGCCCGGGTGTTATCGAGCATGGCAGATGCCGTGCTGGCCAGGGTGTACA
AACAGTCCGATCTCGATACCTTGGCAAAGGAGGCTTCCATTCCCATCATCAACG
GCCTGAGCGACCTGTACCACCCAATCCAAATCCTGGCTGACTACCTGACCCTGC
AAGAGCACTACAGCAGCCTGAAGGGTCTGACCCTGTCATGGATTGGCGATGGA
AACAATATTCTGCACTCCATCATGATGTCCGCCGCGAAGTTCGGAATGCATCTG
CAAGCCGCCACTCCAAAAGGATACGAACCGGATGCATCCGTGACCAAGTTGGC
GGAACAGTACGCGAAGGAGAACGGAACCAAGCTCCTGCTGACTAACGACCCGC
TCGAGGCTGCGCATGGGGGTAACGTGCTGATTACGGACACCTGGATCTCCATGG
GGCAGGAGGAAGAGAAGAAGAAGAGACTGCAGGCATTCCAGGGGTACCAGGT
CACCATGAAAACCGCAAAAGTGGCAGCTTCGGACTGGACTTTCCTGCATTGCCT
GCCGAGGAAGCCGGAGGAAGTCGACGACGAAGTGTTCTACTCGCCTCGGTCCC
TGGTGTTCCCCGAGGCCGAAAACCGGAAGTGGACCATCATGGCCGTGATGGTG
TCCTTGCTGACTGACTATAGCCCGCAGCTGCAGAAGCCTAAGTTCTAG
>pARM711
(SEQ ID NO: 144)
ATGCTTTTCAACCTGAGAATCCTCTTGAACAATGCTGCTTTTCGGAATGGCCAC
AACTTTATGGTTCGGAACTTCCGTTGCGGCCAGCCTTTACAAAACAAGGTCCAG
CTGAAGGGCCGGGATTTGCTCACACTAAAGAACTTTACTGGAGAAGAGATCAA
GTACATGCTATGGCTGTCGGCCGACCTGAAGTTCCGTATCAAGCAGAAGGGAG
AATACCTTCCGCTGCTTCAAGGAAAGAGCCTCGGCATGATCTTTGAGAAGCGCT
CAACCAGGACCCGCCTTTCTACTGAAACTGGGTTCGCGCTGCTCGGTGGCCACC
CCTGCTTCCTGACGACCCAGGACATCCACCTCGGAGTGAACGAATCCCTCACCG
ATACCGCCCGGGTGTTATCGAGCATGGCAGATGCCGTGCTGGCCAGGGTGTACA
AACAGTCCGATCTCGATACCTTGGCAAAGGAGGCTTCCATTCCCATCATCAACG
GCCTGAGCGACCTGTACCACCCAATCCAAATCCTGGCTGACTACCTGACCCTGC
AAGAGCACTACAGCAGCCTGAAGGGTCTGACCCTGTCATGGATTGGCGATGGA
AACAATATTCTGCACTCCATCATGATGTCCGCCGCGAAGTTCGGAATGCATCTG
CAAGCCGCCACTCCAAAAGGATACGAACCGGATGCGTCCGTGACCAAGTTGGC
GGAACAGTACGCGAAGGAGAACGGAACCAAGCTTCTGCTGACTAACGACCCCC
TCGAGGCTGCGCATGGGGGCAACGTGCTGATTACCGACACCTGGATCTCCATGG
GGCAGGAGGAAGAGAAGAAGAAGAGACTGCAGGCATTCCAGGGGTACCAGGT
CACCATGAAAACCGCAAAAGTGGCAGCTTCGGACTGGACTTTCCTGCATTGCCT
GCCGAGGAAGCCGGAGGAAGTCGACGACGAAGTGTTCTACTCGCCTCGGTCCC
TGGTGTTCCCCGAGGCCGAAAACCGGAAGTGGACCATCATGGCCGTGATGGTG
TCCTTGCTGACTGACTATAGCCCGCAGCTGCAGAAGCCTAAGTTCTAG
>pARM712
(SEQ ID NO: 145)
ATGCTGTTCAACCTGCGAATCCTGCTGAACAACGCCGCTTTTCGGAACGGGCAC
AACTTTATGGTGAGGAACTTTCGCTGCGGACAGCCCCTCCAGAATAAGGTCCAG
CTGAAGGGCAGGGACCTGCTGACCCTGAAAAATTTCACAGGGGAGGAAATCAA
GTATATGCTGTGGCTGTCAGCTGATCTGAAGTTCCGGATCAAGCAGAAGGGCGA
ATATCTGCCTCTGCTCCAGGGCAAAAGCCTGGGGATGATCTTCGAAAAGCGCAG
TACTCGGACCAGACTGTCAACCGAGACTGGATTCGCTCTGCTGGGAGGACACCC
TTGTTTTCTGACCACTCAGGACATTCACCTGGGAGTGAACGAGTCCCTGACCGA
CACTGCTCGCGTCCTGAGCTCTATGGCCGACGCTGTGCTGGCTCGAGTCTACAA
ACAGTCCGACCTGGATACCCTGGCCAAGGAAGCTTCTATCCCAATTATTAACGG
CCTGTCAGACCTGTATCACCCCATCCAGATTCTGGCCGATTACCTGACCCTCCA
GGAGCACTATTCTAGTCTGAAAGGGCTGACACTGAGTTGGATTGGGGACGGAA
ACAATATCCTGCACTCTATTATGATGTCAGCCGCCAAGTTTGGAATGCACCTCC
AGGCTGCAACCCCAAAAGGCTACGAACCCGATGCCTCAGTGACAAAGCTGGCT
GAACAGTACGCCAAAGAGAACGGCACTAAGCTGCTGCTGACCAACGACCCTCT
GGAGGCCGCTCACGGAGGCAACGTGCTGATCACCGATACCTGGATTAGTATGG
GACAGGAGGAAGAGAAGAAGAAGCGGCTCCAGGCCTTCCAGGGCTACCAGGT
GACAATGAAAACCGCTAAGGTCGCAGCCAGCGATTGGACCTTTCTGCACTGCCT
GCCCAGAAAGCCCGAAGAGGTGGACGACGAGGTCTTCTACTCTCCCAGAAGCC
TGGTGTTTCCCGAAGCTGAGAATAGGAAGTGGACAATTATGGCAGTGATGGTC
AGCCTGCTGACTGATTATTCACCTCAGCTCCAGAAACCAAAGTTCTGA
>pARM713
(SEQ ID NO: 146)
ATGCTGTTCAACCTGCGCATCCTGCTGAACAACGCCGCCTTCCGCAACGGCCAC
AACTTCATGGTGCGCAACTTCCGCTGCGGCCAGCCCCTGCAGAACAAGGTGCAG
CTGAAGGGCCGCGACCTGCTGACCCTGAAGAACTTCACCGGCGAGGAGATCAA
GTACATGCTGTGGCTGAGCGCCGACCTGAAGTTCCGCATCAAGCAGAAGGGCG
AGTACCTGCCCCTGCTGCAGGGCAAGAGCCTGGGCATGATCTTCGAGAAGCGC
AGCACCCGCACCCGCCTGAGCACCGAGACAGGCCTGGCCCTGCTGGGCGGCCA
CCCCTGCTTCCTGACCACCCAGGACATCCACCTGGGCGTGAACGAGAGCCTGAC
CGACACCGCCCGCGTGCTGAGCAGCATGGCCGACGCCGTGCTGGCCCGCGTGT
ACAAGCAGAGCGACCTGGACACCCTGGCCAAGGAGGCCAGCATCCCCATCATC
AACGGCCTGAGCGACCTGTACCACCCCATCCAGATCCTGGCCGACTACCTGACC
CTGCAGGAGCACTACAGCAGCCTGAAGGGCCTGACCCTGAGCTGGATCGGCGA
CGGCAACAACATCCTGCACAGCATCATGATGAGCGCCGCCAAGTTCGGCATGC
ACCTGCAGGCCGCCACCCCCAAGGGCTACGAGCCCGACGCCAGCGTGACCAAG
CTGGCCGAGCAGTACGCCAAGGAGAACGGCACCAAGCTGCTGCTGACCAACGA
CCCCCTGGAGGCCGCCCACGGCGGCAACGTGCTGATCACCGACACCTGGATCA
GCATGGGCCAGGAGGAGGAGAAGAAGAAGCGCCTGCAGGCCTTCCAGGGCTAC
CAGGTGACCATGAAGACCGCCAAGGTGGCCGCCAGCGACTGGACCTTCCTGCA
CTGCCTGCCCCGCAAGCCCGAGGAGGTGGACGACGAGGTGTTCTACAGCCCCC
GCAGCCTGGTGTTCCCCGAGGCCGAGAACCGCAAGTGGACCATCATGGCCGTG
ATGGTGAGCCTGCTGACCGACTACAGCCCCCAGCTGCAGAAGCCCAAGTTCTGA
>pARM714
(SEQ ID NO: 147)
ATGCTGTTCAACCTGCGAATCCTGCTGAACAACGCCGCTTTTCGGAACGGGCAC
AACTTTATGGTGAGGAACTTTCGCTGCGGACAGCCCCTCCAGAATAAGGTCCAG
CTGAAGGGCAGGGACCTGCTGACCCTGAAAAATTTCACAGGGGAGGAAATCAA
GTATATGCTGTGGCTGTCAGCTGATCTGAAGTTCCGGATCAAGCAGAAGGGCGA
ATATCTGCCTCTGCTCCAGGGCAAAAGCCTGGGGATGATCTTCGAAAAGCGCAG
TACTCGGACCAGACTGTCAACCGAGACTGGATTCGCTCTGCTGGGAGGACACCC
TTGTTTTCTGACCACTCAGGACATTCACCTGGGAGTGAACGAGTCCCTGACCGA
CACTGCTCGCGTCCTGAGCTCTATGGCCGACGCTGTGCTAGCTCGAGTCTACAA
ACAGTCCGACCTGGATACCCTGGCCAAGGAAGCTTCTATCCCAATTATTAACGG
CCTGTCAGACCTGTATCACCCCATCCAGATTCTGGCCGATTACCTGACCCTCCA
GGAGCACTATTCTAGTCTGAAAGGGCTGACACTGAGTTGGATTGGGGACGGAA
ACAATATCCTGCACTCTATTATGATGTCAGCCGCCAAGTTTGGAATGCACCTCC
AGGCTGCAACCCCAAAAGGCTACGAACCCGATGCCTCAGTGACAAAGCTGGCT
GAACAGTACGCCAAAGAGAACGGCACTAAGCTGCTGCTGACCAACGACCCTCT
GGAGGCCGCTCACGGAGGCAACGTGCTGATCACCGATACCTGGATTAGTATGG
GACAGGAGGAAGAGAAGAAGAAGCGGCTCCAGGCCTTCCAGGGCTACCAGGT
GACAATGAAAACCGCTAAGGTCGCAGCCAGCGATTGGACCTTTCTGCACTGCCT
GCCCAGAAAGCCCGAAGAGGTGGACGACGAGGTCTTCTACTCTCCCAGAAGCC
TGGTGTTTCCCGAAGCTGAGAATAGGAAGTGGACAATTATGGCAGTGATGGTC
AGCCTGCTGACTGATTATTCACCTCAGCTCCAGAAACCAAAGTTCTGA
>pARM715
(SEQ ID NO: 148)
ATGCTGTTCAACCTGCGAATCCTGCTGAACAATGCCGCTTTTCGGAACGGGCAC
AATTTCATGGTGAGGAACTTTCGCTGCGGACAGCCCCTCCAGAACAAGGTCCAG
CTGAAGGGCAGGGACCTGCTGACCCTGAAAAATTTCACAGGGGAGGAAATCAA
GTACATGCTGTGGCTGTCAGCCGATCTGAAGTTCCGGATCAAGCAGAAGGGCG
AATATCTGCCTCTGCTCCAGGGCAAAAGCCTGGGGATGATCTTCGAAAAGCGCA
GTACTCGGACCAGACTGTCAACAGAGACTGGATTCGCACTGCTGGGAGGACAC
CCATGTTTTCTGACCACACAGGACATTCATCTGGGAGTGAACGAGTCCCTGACC
GACACAGCACGCGTCCTGAGCTCCATGGCTGATGCAGTGCTGGCTCGAGTCTAC
AAACAGTCTGACCTGGATACCCTGGCCAAGGAAGCTTCTATCCCAATCATTAAT
GGCCTGAGTGACCTGTATCACCCCATCCAGATTCTGGCCGATTACCTGACCCTC
CAGGAGCATTATTCTAGTCTGAAAGGGCTGACACTGAGCTGGATTGGGGACGG
AAACAATATCCTGCACTCCATTATGATGAGCGCCGCCAAGTTTGGAATGCACCT
CCAGGCTGCAACCCCAAAAGGCTACGAACCCGATGCCTCCGTGACAAAGCTGG
CAGAACAGTATGCCAAAGAGAACGGCACTAAGCTGCTGCTGACCAATGACCCT
CTGGAGGCCGCTCACGGAGGCAACGTGCTGATCACTGATACCTGGATTAGTATG
GGACAGGAGGAAGAGAAGAAGAAGCGGCTCCAGGCCTTCCAGGGCTACCAGG
TGACAATGAAAACTGCTAAGGTCGCAGCCAGCGACTGGACCTTTCTGCATTGCC
TGCCCAGAAAGCCTGAAGAGGTGGACGATGAGGTCTTCTACTCACCCAGAAGC
CTGGTGTTTCCTGAAGCTGAGAATAGGAAGTGGACAATCATGGCAGTGATGGTC
AGCCTGCTGACTGATTATTCCCCTCAGCTCCAGAAACCAAAGTTCTGA
>pARM716
(SEQ ID NO: 149)
ATGCTTTTCAACCTTCGCATTCTCCTCAACAACGCCGCGTTTAGAAACGGACAC
AACTTCATGGTCCGCAACTTCCGCTGCGGACAGCCGCTGCAGAACAAGGTCCAG
CTCAAGGGTCGGGATCTCCTGACGCTGAAGAACTTTACCGGCGAAGAGATTAA
GTACATGCTGTGGCTGTCCGCCGACCTTAAGTTCCGGATCAAGCAGAAGGGCGA
ATACCTTCCCCTGCTGCAAGGAAAGTCCCTGGGCATGATCTTCGAGAAGCGCAG
TACCAGAACCAGACTCTCCACTGAAACCGGGTTCGCGCTGCTTGGCGGCCACCC
GTGTTTCCTCACTACGCAAGACATCCATCTTGGCGTGAACGAGTCCCTTACCGA
CACCGCCAGGGTGCTGTCAAGCATGGCCGACGCCGTCCTTGCGCGCGTGTACAA
GCAGTCAGACCTTGATACTCTGGCCAAGGAAGCCTCCATCCCTATTATCAACGG
CCTATCCGACCTTTACCACCCGATCCAGATCCTCGCTGACTACCTGACCCTGCA
AGAACACTACAGCAGCCTCAAGGGACTGACTCTGTCCTGGATCGGCGACGGGA
ACAACATCCTGCACTCAATCATGATGAGCGCAGCCAAGTTCGGCATGCATCTCC
AAGCCGCTACACCCAAGGGTTATGAACCGGACGCCTCTGTGACCAAGTTGGCA
GAACAGTACGCCAAGGAGAACGGTACTAAGCTCCTTTTAACCAACGACCCCCTC
GAAGCAGCCCATGGCGGGAATGTGCTCATTACCGATACCTGGATTTCGATGGGC
CAGGAGGAGGAGAAGAAGAAGCGGCTGCAGGCGTTCCAGGGCTACCAGGTCA
CCATGAAAACTGCCAAAGTGGCCGCCTCGGATTGGACCTTTCTCCACTGCCTGC
CTCGGAAGCCTGAGGAGGTGGACGACGAAGTGTTCTACTCCCCACGGTCCCTCG
TGTTCCCCGAGGCCGAAAATAGGAAGTGGACCATCATGGCCGTGATGGTGTCCC
TCTTGACCGATTACAGCCCGCAGCTTCAGAAGCCTAAATTCTAG
>pARM717
(SEQ ID NO: 150)
ATGCTTTTCAATCTTCGCATCCTGTTGAACAACGCCGCCTTCCGCAATGGTCACA
ACTTCATGGTCCGGAACTTCAGATGTGGACAGCCTCTCCAAAACAAGGTCCAGC
TGAAGGGAAGGGACCTCTTAACCCTCAAAAACTTTACTGGAGAGGAGATCAAG
TACATGCTGTGGCTTAGCGCCGACCTTAAGTTCCGGATCAAGCAGAAGGGAGA
GTACCTCCCGCTGCTGCAAGGAAAGAGTCTTGGAATGATCTTCGAGAAGCGGTC
CACCAGAACTCGCCTCTCCACTGAAACCGGATTCGCACTCCTGGGTGGACACCC
GTGCTTTCTGACCACCCAAGACATCCACCTCGGAGTGAACGAGAGCCTCACGGA
CACCGCGAGAGTGCTGTCATCCATGGCCGACGCCGTGCTTGCACGGGTCTACAA
GCAGTCCGATCTGGACACTCTTGCCAAGGAAGCCTCCATTCCTATCATTAACGG
TCTGTCGGATCTGTACCACCCGATTCAGATCCTTGCGGACTACCTCACACTTCAA
GAACACTATTCAAGCCTAAAGGGTCTGACCCTGTCCTGGATCGGAGATGGAAA
CAACATTCTCCATTCCATCATGATGAGCGCTGCCAAGTTCGGAATGCATCTCCA
AGCAGCGACTCCTAAGGGTTACGAGCCGGACGCCTCAGTGACTAAGCTGGCCG
AGCAGTACGCCAAGGAGAACGGTACCAAACTGTTGCTTACTAACGACCCGCTT
GAAGCGGCCCATGGAGGAAACGTGCTGATTACCGACACCTGGATTTCGATGGG
ACAGGAAGAGGAGAAGAAGAAGCGGCTCCAGGCGTTCCAGGGATACCAGGTC
ACCATGAAAACGGCCAAAGTGGCCGCTAGCGATTGGACCTTTCTGCACTGCCTC
CCGCGCAAGCCTGAAGAAGTGGACGACGAAGTGTTCTACTCCCCTCGCTCTCTT
GTGTTCCCGGAAGCCGAAAACAGGAAGTGGACCATCATGGCCGTGATGGTGTC
CCTCCTGACCGATTACAGCCCGCAGCTGCAGAAGCCTAAGTTCTAG
>pARM718
(SEQ ID NO: 151)
ATGCTTTTCAATCTCCGCATCCTCCTCAACAACGCCGCGTTTAGAAACGGCCAC
AACTTCATGGTCCGGAACTTCAGATGTGGCCAGCCGCTTCAGAACAAGGTCCAG
CTCAAGGGCCGGGATCTTCTGACCCTGAAGAACTTTACTGGCGAAGAAATCAA
GTACATGCTCTGGCTCTCCGCCGACTTGAAGTTCCGCATTAAGCAGAAGGGGGA
ATACCTTCCGCTGCTGCAAGGAAAGTCGCTCGGCATGATCTTTGAGAAGCGCTC
AACCCGCACCAGGCTGTCCACTGAAACCGGGTTCGCGCTGCTTGGTGGCCACCC
CTGCTTCCTGACCACCCAAGACATTCACCTCGGAGTGAACGAATCGCTCACTGA
TACTGCCCGGGTGCTGTCGTCGATGGCCGATGCAGTGCTGGCCAGGGTGTACAA
ACAGTCCGATCTGGACACTCTGGCCAAGGAGGCGTCCATCCCTATTATCAACGG
CCTTTCCGACCTCTACCACCCGATTCAGATCCTTGCCGATTACCTCACCCTGCAA
GAACACTACTCGTCACTGAAGGGTCTGACCTTGTCCTGGATCGGCGACGGCAAC
AACATCCTCCATTCCATTATGATGTCCGCCGCCAAATTCGGCATGCATCTTCAA
GCCGCAACCCCTAAGGGTTACGAGCCGGACGCTTCCGTGACCAAGCTCGCCGA
GCAGTACGCTAAGGAGAACGGAACCAAGCTTCTGCTGACTAACGACCCCCTAG
AGGCAGCCCACGGGGGCAACGTGCTTATTACTGACACCTGGATCTCCATGGGAC
AGGAAGAAGAGAAGAAGAAGCGGTTACAGGCGTTCCAGGGCTATCAGGTCACC
ATGAAAACCGCCAAGGTCGCTGCCTCGGACTGGACCTTCCTGCATTGCCTGCCT
CGCAAGCCCGAAGAAGTGGACGACGAGGTGTTCTACTCGCCACGGTCCCTTGTG
TTCCCTGAGGCCGAGAATAGAAAGTGGACCATTATGGCCGTGATGGTGTCCCTT
CTCACCGACTACTCGCCGCAACTGCAGAAACCCAAGTTCTAG
>pARM719
(SEQ ID NO: 152)
ATGCTTTTCAATCTTCGCATCCTCCTCAACAACGCCGCCTTCCGGAACGGTCACA
ACTTCATGGTCCGGAACTTCCGCTGCGGCCAGCCGCTCCAAAACAAAGTGCAGC
TTAAGGGCCGCGATCTCCTGACCCTGAAGAACTTCACCGGAGAGGAAATCAAG
TACATGCTGTGGCTCTCGGCGGACCTGAAGTTTAGGATTAAGCAGAAGGGGGA
GTATCTGCCGCTGCTCCAAGGGAAGTCCCTTGGCATGATCTTCGAAAAGAGGTC
CACCCGGACTCGGCTCAGCACCGAAACAGGTTTTGCACTTCTGGGGGGCCACCC
GTGCTTCCTGACGACCCAGGACATCCATCTGGGTGTCAACGAGAGTTTGACCGA
CACTGCCAGAGTGCTGTCATCCATGGCGGACGCGGTGCTCGCGAGAGTGTACA
AGCAGTCCGATCTTGACACCCTGGCAAAAGAGGCTTCAATCCCGATCATTAACG
GACTCTCGGATCTGTACCACCCTATCCAAATCTTGGCCGACTACCTGACCCTGC
AAGAACACTACAGCTCCCTGAAGGGCCTGACTCTTTCCTGGATTGGCGATGGAA
ACAACATTCTCCATTCTATTATGATGTCCGCCGCCAAGTTCGGCATGCACCTTCA
AGCCGCCACCCCGAAGGGCTACGAACCTGACGCCTCCGTGACTAAGCTAGCCG
AACAGTACGCTAAGGAGAACGGCACTAAGCTTCTCCTTACCAACGATCCGCTGG
AGGCGGCCCATGGCGGAAATGTGCTTATCACCGACACCTGGATTAGCATGGGG
CAGGAAGAAGAGAAGAAGAAACGGCTCCAGGCATTCCAGGGCTACCAGGTCAC
CATGAAAACTGCCAAGGTCGCCGCTAGCGACTGGACCTTCCTCCACTGTCTGCC
TCGCAAGCCTGAAGAAGTGGACGACGAGGTGTTCTACTCCCCGCGCTCCCTCGT
GTTTCCTGAGGCCGAGAACAGAAAGTGGACCATCATGGCCGTGATGGTGTCATT
ACTTACGGACTACAGCCCGCAGCTGCAGAAGCCGAAGTTCTAG
>pARM720
(SEQ ID NO: 153)
ATGCTTTTTAACTTGAGAATCCTTCTGAACAACGCCGCTTTCCGCAACGGTCATA
ACTTCATGGTCCGGAACTTCAGATGTGGCCAGCCCCTCCAAAACAAAGTGCAGC
TGAAGGGCCGGGACCTTCTTACGCTGAAGAATTTCACCGGCGAAGAAATCAAG
TACATGCTCTGGCTGTCCGCCGATCTTAAGTTCCGCATTAAGCAGAAGGGGGAA
TACCTCCCGCTGCTGCAAGGGAAGTCGCTGGGCATGATTTTTGAGAAGCGGTCA
ACTCGCACCCGCCTGTCCACTGAAACTGGATTCGCACTGCTCGGTGGCCATCCC
TGCTTCCTGACCACCCAAGACATCCACCTCGGCGTGAACGAGTCCCTGACTGAC
ACCGCCCGGGTCTTATCCTCGATGGCCGATGCTGTGCTTGCGAGGGTGTACAAG
CAGTCCGACCTCGACACACTCGCGAAGGAGGCCTCCATCCCCATCATCAACGGC
CTGTCCGACCTTTACCACCCAATTCAGATCCTCGCCGATTACCTGACCCTGCAA
GAGCACTACTCGTCGCTCAAGGGGCTTACCCTCTCGTGGATTGGCGACGGCAAC
AACATCCTTCACTCCATCATGATGTCGGCAGCGAAGTTCGGCATGCATCTGCAA
GCCGCCACGCCTAAGGGTTATGAACCGGATGCCTCAGTGACCAAGCTCGCCGA
ACAGTACGCGAAAGAGAATGGAACCAAGCTACTTCTGACCAACGACCCCCTGG
AGGCCGCTCACGGCGGCAACGTCCTCATTACCGATACTTGGATTTCGATGGGAC
AGGAAGAGGAAAAGAAGAAGAGACTGCAGGCGTTCCAGGGATACCAGGTCAC
CATGAAAACTGCCAAAGTGGCAGCCTCCGACTGGACCTTCCTTCACTGCCTGCC
GAGGAAGCCTGAAGAGGTGGACGACGAGGTGTTCTACTCCCCGCGCTCCTTGGT
GTTTCCTGAGGCCGAAAACCGGAAGTGGACTATCATGGCCGTGATGGTGTCCCT
CCTCACCGACTACTCGCCGCAACTGCAGAAGCCTAAGTTCTAG
>pARM721
(SEQ ID NO: 154)
ATGTTATTCAACCTTAGAATTCTCCTTAACAACGCCGCCTTCCGGAATGGGCAT
AACTTTATGGTCCGCAATTTCCGCTGTGGACAGCCTCTGCAAAACAAGGTCCAG
CTCAAGGGCCGGGATCTGCTGACTCTCAAGAACTTCACTGGGGAAGAAATCAA
GTACATGCTCTGGCTGAGCGCCGACCTCAAGTTCCGCATCAAGCAGAAGGGAG
AGTACCTCCCGCTGCTCCAAGGGAAGTCCCTGGGCATGATCTTCGAGAAGAGAT
CCACCCGCACCAGACTTTCCACTGAGACTGGCTTCGCCTTGCTGGGAGGCCACC
CATGCTTCCTGACGACCCAGGACATTCACCTTGGCGTGAACGAGTCCCTGACTG
ACACCGCAAGGGTGTTGTCCTCGATGGCCGACGCCGTGCTTGCCCGGGTGTACA
AGCAGAGCGATCTTGACACCCTGGCTAAGGAAGCTTCCATTCCCATCATCAACG
GTCTGAGCGACCTGTACCACCCGATTCAGATCCTGGCGGACTACCTAACCCTGC
AAGAGCACTATAGCTCCCTGAAGGGCCTCACACTTTCATGGATCGGCGACGGCA
ACAACATCCTGCACTCTATTATGATGAGCGCTGCCAAATTCGGCATGCACCTCC
AAGCCGCCACGCCTAAAGGCTACGAGCCCGACGCCTCGGTGACCAAGCTTGCG
GAGCAGTACGCGAAGGAAAACGGCACCAAGCTGCTTCTCACCAACGATCCTCT
GGAAGCGGCCCATGGTGGCAACGTGCTCATTACCGACACTTGGATCTCCATGGG
ACAGGAGGAGGAAAAGAAGAAGCGGCTCCAGGCGTTTCAGGGTTACCAGGTCA
CCATGAAAACCGCCAAGGTCGCAGCCTCCGACTGGACCTTCCTTCATTGCCTTC
CGCGCAAGCCCGAAGAAGTGGACGATGAAGTGTTTTACTCACCTCGGTCACTCG
TGTTCCCGGAAGCAGAGAACAGGAAATGGACCATTATGGCCGTGATGGTGTCC
CTGCTCACCGATTACAGTCCGCAACTGCAGAAGCCCAAGTTCTAG
>pARM722
(SEQ ID NO: 155)
ATGCTTTTCAATCTCCGCATCCTCCTTAACAACGCCGCGTTTAGAAACGGCCAC
AACTTCATGGTCCGGAACTTCAGATGTGGCCAGCCGCTTCAAAACAAGGTCCAG
CTGAAGGGCCGGGATCTTCTGACCCTGAAGAACTTTACTGGCGAAGAGATCAA
GTACATGCTCTGGCTCTCCGCGGACTTGAAGTTCCGCATTAAGCAGAAGGGGGA
ATACCTTCCGCTGCTTCAAGGAAAGAGCCTCGGCATGATCTTTGAGAAGCGCTC
AACCAGGACCCGCCTTTCTACTGAAACTGGGTTCGCGCTGCTCGGTGGCCACCC
CTGCTTCCTGACGACCCAGGACATCCACCTCGGAGTGAACGAATCCCTCACCGA
TACCGCCCGGGTGTTATCGAGCATGGCAGATGCCGTGCTGGCCAGGGTGTACAA
ACAGTCCGATCTGGACACTCTGGCCAAGGAGGCGTCAATTCCTATTATCAACGG
CCTTAGTGACCTCTACCATCCGATTCAAATCCTGGCCGATTACCTCACCCTGCAA
GAACACTACAGCTCCCTGAAGGGTCTGACATTGTCCTGGATCGGCGACGGCAAC
AACATTCTCCATTCCATCATGATGTCCGCCGCAAAATTCGGCATGCATCTTCAA
GCCGCCACGCCTAAGGGTTACGAACCCGACGCTTCCGTGACTAAGCTCGCCGAG
CAGTACGCTAAGGAGAACGGAACCAAGCTGCTGCTGACTAACGACCCGCTAGA
AGCAGCCCACGGGGGCAACGTGCTTATTACTGACACCTGGATCTCCATGGGCCA
GGAGGAAGAGAAAAAGAAGCGGCTGCAGGCGTTCCAGGGATATCAGGTCACC
ATGAAAACCGCCAAGGTCGCTGCCTCCGACTGGACCTTCCTGCACTGCCTGCCT
CGCAAGCCTGAAGAAGTGGACGACGAGGTGTTCTACTCGCCACGGAGCCTCGT
GTTCCCCGAGGCCGAGAATAGAAAGTGGACCATCATGGCCGTGATGGTGTCAC
TTCTCACCGACTACAGCCCGCAGCTTCAGAAGCCCAAGTTCTAG
>pARM723
(SEQ ID NO: 156)
ATGCTTTTCAATCTCCGCATCCTCCTTAACAACGCCGCGTTTAGAAACGGCCAC
AACTTCATGGTCCGGAACTTCAGATGTGGCCAGCCGCTTCAAAACAAGGTCCAG
CTTAAGGGCCGGGATCTCCTCACCCTTAAAAACTTCACCGGCGAAGAGATCAAG
TACATGCTCTGGCTCTCCGCGGACCTTAAGTTCCGCATTAAGCAGAAGGGGGAA
TACCTTCCGCTGCTTCAAGGAAAGAGCCTCGGCATGATCTTTGAGAAGCGCTCA
ACCAGGACCAGGCTTTCTACTGAAACTGGGTTCGCGCTTCTCGGCGGTCATCCC
TGCTTCCTCACGACCCAAGACATCCACCTCGGAGTGAACGAATCCCTCACGGAT
ACTGCCCGCGTGCTTTCGAGCATGGCAGACGCCGTGCTCGCCCGGGTGTACAAA
CAGTCCGATCTCGACACTCTCGCCAAGGAGGCGTCAATTCCTATTATCAACGGT
CTTAGTGACCTTTACCACCCGATCCAGATCCTCGCCGATTACCTCACACTCCAA
GAACACTACAGCTCCCTTAAGGGTCTTACCCTCTCCTGGATCGGCGACGGCAAC
AACATTCTCCACTCCATCATGATGTCCGCCGCAAAGTTCGGCATGCATCTTCAA
GCCGCCACCCCGAAGGGCTACGAGCCTGATGCTTCCGTGACTAAGCTCGCCGAG
CAGTACGCTAAGGAGAACGGAACCAAGCTTCTTCTCACTAACGACCCACTCGA
AGCAGCCCATGGGGGCAACGTGCTTATCACTGACACCTGGATCTCCATGGGCCA
GGAAGAAGAGAAGAAGAAGCGGCTCCAGGCGTTCCAGGGATATCAGGTCACCA
TGAAAACCGCCAAGGTCGCTGCCTCCGACTGGACCTTTCTCCACTGCCTCCCTC
GCAAACCTGAAGAAGTGGACGACGAGGTGTTCTACTCGCCCCGGAGCCTCGTG
TTCCCCGAGGCCGAGAATAGAAAGTGGACCATTATGGCCGTGATGGTGTCACTC
CTCACCGACTACAGCCCGCAGCTTCAGAAGCCCAAGTTCTAG
>pARM724
(SEQ ID NO: 157)
ATGCTTTTCAATCTCCGCATCCTCCTTAACAACGCCGCGTTTAGAAACGGACAT
AACTTCATGGTCCGGAACTTCAGATGTGGACAGCCGCTTCAAAACAAGGTCCAG
CTGAAGGGTCGGGATCTTCTGACCCTGAAGAACTTTACCGGAGAAGAGATCAA
GTACATGCTCTGGCTCTCCGCGGACTTGAAGTTCCGCATTAAGCAGAAGGGAGA
ATACCTCCCGCTGCTTCAAGGAAAGAGCCTCGGAATGATTTTTGAGAAGCGCTC
AACCAGGACCCGCCTTTCTACTGAAACTGGATTCGCGCTGCTGGGTGGACACCC
CTGCTTCCTGACGACCCAGGACATCCACCTCGGAGTGAACGAATCCCTCACTGA
TACCGCCCGGGTGTTATCGAGCATGGCAGATGCCGTGCTGGCCAGGGTGTACAA
ACAGTCCGATCTGGACACTCTGGCCAAGGAGGCGTCAATTCCTATCATCAACGG
ACTTAGTGACCTCTACCATCCGATTCAAATCCTGGCCGACTACCTCACCCTGCA
AGAACACTACAGCTCCCTGAAGGGTCTGACATTGTCCTGGATCGGAGATGGAA
ACAACATTCTCCACTCCATCATGATGTCCGCCGCAAAATTCGGAATGCATCTTC
AAGCCGCCACGCCTAAGGGTTACGAACCCGACGCTTCCGTGACTAAGCTCGCCG
AGCAGTACGCTAAGGAGAACGGTACCAAGCTTCTCCTGACCAACGACCCACTA
GAAGCAGCCCACGGTGGAAACGTGCTTATTACTGACACTTGGATCTCCATGGGA
CAGGAGGAAGAGAAAAAGAAGCGGCTGCAGGCGTTCCAGGGATATCAGGTCA
CCATGAAAACCGCCAAGGTCGCTGCCTCCGACTGGACCTTCCTGCACTGCCTGC
CTCGCAAGCCTGAAGAAGTGGACGACGAGGTGTTCTACTCGCCGCGGAGCCTC
GTGTTCCCCGAGGCCGAGAATAGAAAGTGGACCATCATGGCCGTGATGGTGTC
ACTGCTCACCGACTACAGCCCGCAGCTTCAGAAGCCCAAGTTCTAG
>pARM725
(SEQ ID NO: 158)
ATGCTTTTCAACCTCCGCATTCTCCTCAACAACGCTGCCTTCCGGAATGGACATA
ACTTCATGGTCCGGAACTTCAGATGCGGACAGCCGCTTCAGAACAAGGTCCAGC
TTAAGGGGAGAGATCTCCTTACCCTCAAAAACTTCACTGGCGAAGAAATCAAGT
ACATGCTCTGGCTTAGTGCGGATCTCAAGTTCCGCATCAAGCAGAAGGGAGAAT
ACCTCCCGCTCCTTCAAGGAAAGAGCCTCGGCATGATTTTTGAGAAGAGGTCCA
CCAGAACTCGCCTTTCAACCGAGACTGGGTTCGCCCTGCTTGGCGGTCACCCCT
GCTTCCTCACTACCCAAGACATCCACCTCGGCGTGAACGAGAGCCTTACCGACA
CCGCCCGCGTGCTCTCCTCAATGGCCGACGCTGTGCTCGCCCGGGTGTACAAGC
AGTCCGACCTTGATACTCTCGCCAAGGAGGCCTCCATCCCAATTATCAACGGGC
TCTCTGATCTCTACCACCCTATCCAAATCCTCGCGGACTACCTCACCCTCCAAGA
GCACTATAGCTCGCTCAAGGGCCTCACCCTTTCCTGGATTGGCGACGGCAACAA
CATTCTTCACTCGATCATGATGTCCGCCGCCAAGTTCGGCATGCATCTCCAAGC
CGCGACCCCCAAGGGCTACGAGCCTGACGCATCCGTGACCAAGCTCGCCGAGC
AGTACGCGAAGGAAAATGGCACCAAGCTTCTTCTCACCAACGACCCCCTTGAG
GCCGCTCATGGCGGCAACGTGCTCATCACTGACACTTGGATCAGCATGGGCCAG
GAGGAGGAAAAGAAGAAGCGCCTTCAGGCATTCCAGGGTTACCAGGTCACCAT
GAAAACCGCCAAAGTGGCCGCCTCCGACTGGACCTTTCTTCACTGTCTCCCGCG
GAAGCCTGAAGAAGTGGATGACGAAGTGTTTTACTCCCCTCGGTCACTCGTGTT
CCCGGAAGCAGAAAACAGGAAGTGGACCATTATGGCGGTCATGGTGTCCCTCC
TCACCGACTACAGCCCGCAGCTTCAGAAACCCAAGTTCTAG
>pARM726
(SEQ ID NO: 159)
ATGCTTTTCAATCTCCGCATCCTCCTTAACAACGCAGCGTTTAGAAACGGTCAC
AACTTCATGGTCCGGAACTTCCGCTGTGGACAGCCGCTTCAAAACAAGGTCCAG
CTGAAGGGTCGGGACCTTCTGACCCTGAAGAACTTTACTGGAGAAGAGATCAA
GTACATGCTTTGGCTGTCCGCGGACTTGAAGTTCCGCATTAAGCAGAAGGGAGA
ATACCTTCCGCTGCTCCAAGGAAAGAGCCTGGGAATGATCTTTGAGAAGCGCTC
AACCAGGACCCGCCTTTCTACTGAAACTGGATTCGCGCTGCTGGGTGGTCACCC
TTGCTTCCTGACGACCCAGGACATTCACCTCGGAGTGAACGAGTCCCTCACTGA
TACCGCCAGAGTGTTATCGAGCATGGCAGATGCCGTGCTGGCTAGGGTGTACAA
ACAGTCCGATCTGGACACCCTGGCCAAGGAGGCATCAATTCCTATTATCAACGG
ACTTAGTGACCTCTACCATCCGATTCAAATCCTGGCCGATTACCTCACCCTGCA
AGAACACTACAGCTCCCTGAAGGGTCTGACATTGTCCTGGATCGGAGATGGAA
ACAACATTCTCCATTCCATCATGATGTCCGCGGCCAAGTTCGGAATGCATCTCC
AAGCCGCCACGCCGAAAGGATACGAGCCGGACGCTTCCGTGACTAAGCTCGCC
GAGCAGTACGCTAAGGAGAACGGAACCAAGCTTCTGCTGACTAACGACCCGCT
AGAAGCCGCCCACGGTGGAAACGTGCTTATTACTGACACCTGGATCTCCATGGG
ACAGGAAGAAGAGAAAAAGAAGCGGCTGCAGGCGTTCCAGGGATATCAGGTC
ACCATGAAAACCGCCAAGGTCGCCGCCTCCGACTGGACCTTCCTTCACTGCCTG
CCTCGGAAGCCTGAAGAAGTGGACGACGAGGTGTTCTACTCGCCGCGGAGCCT
CGTGTTCCCTGAGGCCGAGAATAGAAAGTGGACCATCATGGCCGTGATGGTGTC
ACTCCTCACCGACTACAGCCCGCAGCTTCAGAAGCCTAAGTTCTAG
>pARM727
(SEQ ID NO: 160)
ATGCTTTTCAATCTCCGCATTCTCCTCAACAACGCAGCCTTTAGAAACGGCCAC
AACTTCATGGTCCGGAACTTCAGATGTGGCCAGCCGCTTCAGAACAAGGTCCAG
CTCAAGGGCCGGGACCTCCTCACCCTCAAAAACTTTACCGGCGAAGAGATCAA
GTACATGCTCTGGCTTTCGGCCGACCTTAAGTTCCGCATCAAGCAGAAGGGGGA
ATACCTTCCGCTGCTTCAAGGAAAGTCCCTCGGCATGATCTTTGAAAAGCGCTC
GACCAGGACCCGCCTTTCCACTGAAACCGGGTTCGCGCTTCTCGGTGGCCACCC
CTGCTTCCTCACCACCCAAGACATTCACCTCGGAGTGAACGAATCCCTTACCGA
TACCGCAAGAGTGCTTTCGTCGATGGCCGATGCCGTGCTTGCGCGGGTGTACAA
GCAGTCAGATCTCGACACTCTCGCCAAGGAGGCGTCCATTCCTATTATCAACGG
CCTTTCCGACCTTTACCACCCGATTCAGATCCTCGCCGATTACCTCACCCTGCAA
GAGCACTACTCGTCACTCAAGGGTCTTACCCTCTCCTGGATCGGCGACGGAAAC
AACATCCTCCATTCGATCATGATGTCCGCCGCCAAATTCGGCATGCACCTCCAA
GCCGCGACCCCGAAGGGTTACGAGCCCGACGCTTCCGTGACCAAGCTCGCCGA
ACAGTACGCTAAGGAAAACGGCACCAAGCTCCTCCTCACTAACGACCCTCTCGA
AGCAGCCCATGGGGGCAACGTGCTCATTACTGACACTTGGATCTCGATGGGCCA
GGAAGAGGAGAAAAAGAAGCGGCTTCAGGCGTTCCAGGGATATCAGGTCACCA
TGAAAACCGCCAAGGTCGCTGCCTCGGACTGGACCTTCCTTCACTGCCTTCCGC
GCAAGCCTGAAGAGGTGGACGATGAGGTGTTCTACTCCCCACGGTCCCTTGTGT
TCCCCGAGGCCGAGAATAGGAAGTGGACCATCATGGCCGTGATGGTGTCGCTC
CTCACTGACTACTCCCCGCAACTTCAGAAGCCTAAGTTCTAG
>pARM728
(SEQ ID NO: 161)
ATGCTGTTTAATCTGAGAATACTTCTAAACAACGCCGCCTTCCGGAATGGCCAT
AACTTTATGGTTCGGAATTTCCGCTGCGGCCAGCCGCTGCAGAACAAGGTCCAG
CTGAAGGGAAGAGACTTGCTGACCCTCAAGAACTTCACCGGAGAAGAAATCAA
GTATATGCTGTGGCTGTCCGCCGACCTGAAATTCCGCATCAAGCAGAAGGGCGA
ATATCTGCCGCTGTTGCAAGGGAAGTCCCTGGGGATGATCTTCGAGAAGAGGTC
CACCAGAACACGGCTTTCAACCGAAACCGGGTTTGCACTGCTGGGTGGACACCC
CTGTTTTCTGACCACTCAAGATATCCACCTGGGCGTGAACGAGTCCCTTACCGA
CACTGCTAGGGTGTTGTCCAGCATGGCCGATGCCGTCCTGGCTCGCGTGTACAA
GCAGTCCGACCTGGATACCCTGGCAAAGGAAGCGTCCATTCCCATTATCAACGG
GCTGTCCGACCTGTACCATCCGATTCAAATCCTGGCGGACTACCTGACTCTGCA
AGAGCATTACAGCAGCTTGAAGGGGCTTACTCTCTCGTGGATCGGCGACGGGA
ACAACATCCTGCACTCCATCATGATGTCCGCCGCCAAGTTCGGGATGCATTTGC
AAGCTGCGACCCCGAAAGGTTACGAGCCCGATGCTAGCGTAACTAAGCTTGCC
GAACAGTACGCCAAAGAGAATGGTACAAAACTGCTTCTGACTAACGACCCGCT
GGAAGCAGCCCACGGCGGGAACGTGCTGATAACCGACACCTGGATTTCAATGG
GGCAGGAGGAAGAGAAGAAGAAGCGACTGCAGGCGTTCCAAGGCTATCAGGTT
ACCATGAAAACCGCCAAAGTGGCAGCCAGCGATTGGACTTTCCTGCACTGTCTG
CCGCGGAAGCCCGAGGAAGTTGATGACGAAGTATTCTACTCACCCCGGAGCCT
CGTGTTCCCCGAGGCCGAAAACCGGAAGTGGACTATTATGGCCGTGATGGTGTC
GCTGTTGACCGACTACAGCCCGCAACTGCAGAAGCCGAAGTTTTAG
>pARM729
(SEQ ID NO: 162)
ATGCTTTTCAACCTGAGGATCCTTTTGAACAACGCCGCCTTTCGCAACGGCCAC
AACTTTATGGTCCGCAATTTCCGCTGCGGGCAGCCGCTGCAGAACAAGGTCCAG
CTGAAGGGCCGGGATCTGCTGACCCTGAAGAACTTCACCGGGGAGGAAATCAA
GTACATGCTTTGGCTCTCCGCCGATCTGAAGTTCAGAATCAAGCAGAAGGGAGA
GTACCTCCCGTTGCTGCAAGGAAAGTCACTCGGAATGATTTTCGAAAAGAGAA
GCACTAGGACCCGCCTCTCAACTGAAACCGGGTTCGCGCTGCTCGGGGGCCATC
CGTGTTTCCTGACTACCCAAGACATCCACCTGGGAGTGAACGAGTCGCTGACCG
ACACCGCACGCGTGCTGTCATCCATGGCGGACGCAGTGCTTGCCCGGGTGTACA
AGCAGTCGGACCTGGACACTCTTGCCAAGGAGGCATCAATCCCCATCATTAACG
GACTGTCCGATCTCTACCACCCGATTCAGATCCTGGCTGACTACCTAACCCTGC
AAGAGCACTACTCAAGCCTGAAGGGGCTGACCCTGTCGTGGATCGGGGACGGC
AACAACATTCTGCACTCCATCATGATGTCGGCGGCTAAGTTCGGGATGCATTTG
CAAGCGGCAACTCCGAAGGGTTATGAACCCGACGCCTCCGTGACCAAGCTGGC
CGAACAGTACGCCAAGGAAAACGGAACCAAGTTGCTGCTGACTAATGATCCCC
TGGAGGCGGCCCACGGGGGGAACGTGCTGATAACCGATACCTGGATCTCCATG
GGGCAGGAAGAAGAGAAGAAAAAGCGGCTGCAGGCATTCCAGGGATACCAGG
TCACCATGAAAACCGCAAAAGTGGCAGCCAGCGACTGGACTTTCCTCCATTGCC
TGCCGCGAAAGCCGGAGGAGGTCGATGACGAGGTGTTCTACTCCCCGCGGTCG
CTGGTGTTCCCGGAGGCGGAAAACCGGAAGTGGACCATTATGGCCGTGATGGT
GTCACTCCTGACTGACTACAGCCCGCAACTGCAGAAGCCGAAGTTCTAG
>pARM1787
(SEQ ID NO: 163)
ATGCTTTTCAATCTCCGCATCCTCCTTAACAACGCCGCGTTTAGAAACGGCCAC
AACTTCATGGTCCGGAACTTCAGATGTGGCCAGCCGCTTCAAAACAAGGTCCAG
CTGAAGGGCCGGGATCTTCTGACCCTGAAGAACTTTACTGGCGAAGAGATCAA
GTACATGCTCTGGCTCTCCGCGGACTTGAAGTTCCGCATTAAGCAGAAGGGGGA
ATACCTTCCGCTGCTTCAAGGAAAGAGCCTCGGCATGATCTTTGAGAAGCGCTC
AACCAGGACCCGCCTTTCTACTGAAACTGGGTTCGCGCTGCTCGGTGGCCACCC
CTGCTTCCTGACGACCCAGGACATCCACCTCGGAGTGAACGAATCCCTCACCGA
TACCGCCCGGGTGTTATCGAGCATGGCAGATGCCGTGCTGGCCAGGGTGTACAA
ACAGTCCGATCTGGACACTCTGGCCAAGGAGGCGTCAATTCCTATTATCAACGG
CCTTAGTGACCTCTACCATCCGATTCAGATCCTGGCCGATTACCTCACCCTGCAA
GAACACTACAGCTCCCTGAAGGGTCTGACATTGTCCTGGATCGGCGACGGCAAC
AACATTCTCCATTCCATCATGATGTCCGCCGCAAAATTCGGCATGCATCTTCAA
GCCGCCACGCCGAAGGGTTACGAGCCCGACGCTTCCGTGACTAAGCTCGCCGA
GCAGTACGCTAAGGAGAACGGAACCAAGCTTCTGCTGACTAACGACCCACTAG
AAGCAGCCCACGGGGGCAACGTGCTTATTACTGACACCTGGATCTCCATGGGCC
AGGAAGAAGAGAAAAAGAAGCGGCTGCAGGCGTTCCAGGGATATCAGGTCAC
CATGAAAACCGCCAAGGTCGCTGCCTCCGACTGGACCTTCCTGCACTGCCTGCC
TCGCAAGCCTGAAGAAGTGGACGACGAGGTGTTCTACTCGCCACGGAGCCTCG
TGTTCCCCGAGGCCGAGAATAGAAAGTGGACCATCATGGCCGTGATGGTGTCA
CTGCTCACCGACTACAGCCCGCAGCTTCAGAAGCCCAAGTTCTAGATAAGTGAA
>pARM1788
(SEQ ID NO: 164)
ATGCTGTTCAACCTGCGCATCCTGCTGAACAACGCCGCCTTCCGCAACGGCCAC
AACTTCATGGTGCGCAACTTCCGCTGCGGCCAGCCCCTGCAGAACAAGGTGCAG
CTGAAGGGCCGCGACCTGCTGACCCTGAAGAACTTCACCGGCGAGGAGATCAA
GTACATGCTGTGGCTGAGCGCCGACCTGAAGTTCCGCATCAAGCAGAAGGGCG
AGTACCTGCCCCTGCTGCAGGGCAAGAGCCTGGGCATGATCTTCGAGAAGCGC
AGCACCCGCACCCGCCTGAGCACCGAGACAGGCCTGGCCCTGCTGGGCGGCCA
CCCCTGCTTCCTGACCACCCAGGACATCCACCTGGGCGTGAACGAGAGCCTGAC
CGACACCGCCCGCGTGCTGAGCAGCATGGCCGACGCCGTGCTGGCCCGCGTGT
ACAAGCAGAGCGACCTGGACACCCTGGCCAAGGAGGCCAGCATCCCCATCATC
AACGGCCTGAGCGACCTGTACCACCCCATCCAGATCCTGGCCGACTACCTGACC
CTGCAGGAGCACTACAGCAGCCTGAAGGGCCTGACCCTGAGCTGGATCGGCGA
CGGCAACAACATCCTGCACAGCATCATGATGAGCGCCGCCAAGTTCGGCATGC
ACCTGCAGGCCGCCACCCCCAAGGGCTACGAGCCCGACGCCAGCGTGACCAAG
CTGGCCGAGCAGTACGCCAAGGAGAACGGCACCAAGCTGCTGCTGACCAACGA
CCCCCTGGAGGCCGCCCACGGCGGCAACGTGCTGATCACCGACACCTGGATCA
GCATGGGCCAGGAGGAGGAGAAGAAGAAGCGCCTGCAGGCCTTCCAGGGCTAC
CAGGTGACCATGAAGACCGCCAAGGTGGCCGCCAGCGACTGGACCTTCCTGCA
CTGCCTGCCCCGCAAGCCCGAGGAGGTGGACGACGAGGTGTTCTACAGCCCCC
GCAGCCTGGTGTTCCCCGAGGCCGAGAACCGCAAGTGGACCATCATGGCCGTG
ATGGTGAGCCTGCTGACCGACTACAGCCCCCAGCTGCAGAAGCCCAAGTTCTGA
ATAAGTGA
>pARM1789
(SEQ ID NO: 165)
ATGCTGTTCAACCTGCGCATCCTGCTGAACAACGCCGCCTTCCGCAACGGCCAC
AACTTCATGGTGCGCAACTTCCGCTGCGGCCAGCCCCTGCAGAACAAGGTGCAG
CTGAAGGGCCGCGACCTGCTGACCCTGAAGAACTTCACCGGCGAGGAGATCAA
GTACATGCTGTGGCTGAGCGCCGACCTGAAGTTCCGCATCAAGCAGAAGGGCG
AGTACCTGCCCCTGCTGCAGGGCAAGAGCCTGGGCATGATCTTCGAGAAGCGC
AGCACCCGCACCCGCCTGAGCACCGAGACAGGCCTGGCCCTGCTGGGCGGCCA
CCCCTGCTTCCTGACCACCCAGGACATCCACCTGGGCGTGAACGAGAGCCTGAC
CGACACCGCCCGCGTGCTGAGCAGCATGGCCGACGCCGTGCTGGCCCGCGTGT
ACAAGCAGAGCGACCTGGACACCCTGGCCAAGGAGGCCAGCATCCCCATCATC
AACGGCCTGAGCGACCTGTACCACCCCATCCAGATCCTGGCCGACTACCTGACC
CTGCAGGAGCACTACAGCAGCCTGAAGGGCCTGACCCTGAGCTGGATCGGCGA
CGGCAACAACATCCTGCACAGCATCATGATGAGCGCCGCCAAGTTCGGCATGC
ACCTGCAGGCCGCCACCCCCAAGGGCTACGAGCCCGACGCCAGCGTGACCAAG
CTGGCCGAGCAGTACGCCAAGGAGAACGGCACCAAGCTGCTGCTGACCAACGA
CCCCCTGGAGGCCGCCCACGGCGGCAACGTGCTGATCACCGACACCTGGATCA
GCATGGGCCAGGAGGAGGAGAAGAAGAAGCGCCTGCAGGCCTTCCAGGGCTAC
CAGGTGACCATGAAGACCGCCAAGGTGGCCGCCAGCGACTGGACCTTCCTGCA
CTGCCTGCCCCGCAAGCCCGAGGAGGTGGACGACGAGGTGTTCTACAGCCCCC
GCAGCCTGGTGTTCCCCGAGGCCGAGAACCGCAAGTGGACCATCATGGCCGTG
ATGGTGAGCCTGCTGACCGACTACAGCCCCCAGCTGCAGAAGCCCAAGTTCTGA
ATAAGTGA
>pARM1790
(SEQ ID NO: 166)
ATGCTTTTCAATCTCCGCATCCTCCTTAACAACGCCGCGTTTAGAAACGGCCAC
AACTTCATGGTCCGGAACTTCAGATGTGGCCAGCCGCTTCAAAACAAGGTCCAG
CTGAAGGGCCGGGATCTTCTGACCCTGAAGAACTTTACTGGCGAAGAGATCAA
GTACATGCTCTGGCTCTCCGCGGACTTGAAGTTCCGCATTAAGCAGAAGGGGGA
ATACCTTCCGCTGCTTCAAGGAAAGAGCCTCGGCATGATCTTTGAGAAGCGCTC
AACCAGGACCCGCCTTTCTACTGAAACTGGGTTCGCGCTGCTCGGTGGCCACCC
CTGCTTCCTGACGACCCAGGACATCCACCTCGGAGTGAACGAATCCCTCACCGA
TACCGCCCGGGTGTTATCGAGCATGGCAGATGCCGTGCTGGCCAGGGTGTACAA
ACAGTCCGATCTGGACACTCTGGCCAAGGAGGCGTCAATTCCTATTATCAACGG
CCTTAGTGACCTCTACCATCCGATTCAGATCCTGGCCGATTACCTCACCCTGCAA
GAACACTACAGCTCCCTGAAGGGTCTGACATTGTCCTGGATCGGCGACGGCAAC
AACATTCTCCATTCCATCATGATGTCCGCCGCAAAATTCGGCATGCATCTTCAA
GCCGCCACGCCGAAGGGTTACGAGCCCGACGCTTCCGTGACTAAGCTCGCCGA
GCAGTACGCTAAGGAGAACGGAACCAAGCTTCTGCTGACTAACGACCCACTAG
AAGCAGCCCACGGGGGCAACGTGCTTATTACTGACACCTGGATCTCCATGGGCC
AGGAAGAAGAGAAAAAGAAGCGGCTGCAGGCGTTCCAGGGATATCAGGTCAC
CATGAAAACCGCCAAGGTCGCTGCCTCCGACTGGACCTTCCTGCACTGCCTGCC
TCGCAAGCCTGAAGAAGTGGACGACGAGGTGTTCTACTCGCCACGGAGCCTCG
TGTTCCCCGAGGCCGAGAATAGAAAGTGGACCATCATGGCCGTGATGGTGTCA
CTGCTCACCGACTACAGCCCGCAGCTTCAGAAGCCCAAGTTCTAGATAAGTGAA
>pARM1791
(SEQ ID NO: 167)
ATGCTTTTCAACCTGAGAATCCTCTTGAACAATGCTGCTTTTCGGAATGGCCAC
AACTTTATGGTTCGGAACTTCCGTTGCGGCCAGCCTTTACAAAACAAGGTCCAG
CTGAAGGGCCGGGATTTGCTCACACTAAAGAACTTTACTGGAGAAGAGATCAA
GTACATGCTATGGCTGTCGGCCGACCTGAAGTTCCGTATCAAGCAGAAGGGAG
AATACCTTCCGCTGCTTCAAGGAAAGAGCCTCGGCATGATCTTTGAGAAGCGCT
CAACCAGGACCCGCCTTTCTACTGAAACTGGGTTCGCGCTGCTCGGTGGCCACC
CCTGCTTCCTGACGACCCAGGACATCCACCTCGGAGTGAACGAATCCCTCACCG
ATACCGCCCGGGTGTTATCGAGCATGGCAGATGCCGTGCTGGCCAGGGTGTACA
AACAGTCCGATCTCGATACCTTGGCAAAGGAGGCTTCCATTCCCATCATCAACG
GCCTGAGCGACCTGTACCACCCAATCCAAATCCTGGCTGACTACCTGACCCTGC
AAGAGCACTACAGCAGCCTGAAGGGTCTGACCCTGTCATGGATTGGCGATGGA
AACAATATTCTGCACTCCATCATGATGTCCGCCGCGAAGTTCGGAATGCATCTG
CAAGCCGCCACTCCAAAAGGATACGAACCGGATGCATCCGTGACCAAGTTGGC
GGAACAGTACGCGAAGGAGAACGGAACCAAGCTCCTGCTGACTAACGACCCGC
TCGAGGCTGCGCATGGGGGTAACGTGCTGATTACGGACACCTGGATCTCCATGG
GGCAGGAGGAAGAGAAGAAGAAGAGACTGCAGGCATTCCAGGGGTACCAGGT
CACCATGAAAACCGCAAAAGTGGCAGCTTCGGACTGGACTTTCCTGCATTGCCT
GCCGAGGAAGCCGGAGGAAGTCGACGACGAAGTGTTCTACTCGCCTCGGTCCC
TGGTGTTCCCCGAGGCCGAAAACCGGAAGTGGACCATCATGGCCGTGATGGTG
TCCTTGCTGACTGACTATAGCCCGCAGCTGCAGAAGCCTAAGTTCTAGATAAGT
GA
>pARM1792
(SEQ ID NO: 168)
ATGCTGTTCAACCTGCGCATCCTGCTGAACAACGCCGCCTTCCGCAACGGCCAC
AACTTCATGGTGCGCAACTTCCGCTGCGGCCAGCCCCTGCAGAACAAGGTGCAG
CTGAAGGGCCGCGACCTGCTGACCCTGAAGAACTTCACCGGCGAGGAGATCAA
GTACATGCTGTGGCTGAGCGCCGACCTGAAGTTCCGCATCAAGCAGAAGGGCG
AGTACCTGCCCCTGCTGCAGGGCAAGAGCCTGGGCATGATCTTCGAGAAGCGC
AGCACCCGCACCCGCCTGAGCACCGAGACAGGCCTGGCCCTGCTGGGCGGCCA
CCCCTGCTTCCTGACCACCCAGGACATCCACCTGGGCGTGAACGAGAGCCTGAC
CGACACCGCCCGCGTGCTGAGCAGCATGGCCGACGCCGTGCTGGCCCGCGTGT
ACAAGCAGAGCGACCTGGACACCCTGGCCAAGGAGGCCAGCATCCCCATCATC
AACGGCCTGAGCGACCTGTACCACCCCATCCAGATCCTGGCCGACTACCTGACC
CTGCAGGAGCACTACAGCAGCCTGAAGGGCCTGACCCTGAGCTGGATCGGCGA
CGGCAACAACATCCTGCACAGCATCATGATGAGCGCCGCCAAGTTCGGCATGC
ACCTGCAGGCCGCCACCCCCAAGGGCTACGAGCCCGACGCCAGCGTGACCAAG
CTGGCCGAGCAGTACGCCAAGGAGAACGGCACCAAGCTGCTGCTGACCAACGA
CCCCCTGGAGGCCGCCCACGGCGGCAACGTGCTGATCACCGACACCTGGATCA
GCATGGGCCAGGAGGAGGAGAAGAAGAAGCGCCTGCAGGCCTTCCAGGGCTAC
CAGGTGACCATGAAGACCGCCAAGGTGGCCGCCAGCGACTGGACCTTCCTGCA
CTGCCTGCCCCGCAAGCCCGAGGAGGTGGACGACGAGGTGTTCTACAGCCCCC
GCAGCCTGGTGTTCCCCGAGGCCGAGAACCGCAAGTGGACCATCATGGCCGTG
ATGGTGAGCCTGCTGACCGACTACAGCCCCCAGCTGCAGAAGCCCAAGTTCTGA
ATAAGTGA
>pARM1793
(SEQ ID NO: 169)
ATGCTTTTCAATCTCCGCATCCTCCTTAACAACGCCGCGTTTAGAAACGGCCAC
AACTTCATGGTCCGGAACTTCAGATGTGGCCAGCCGCTTCAAAACAAGGTCCAG
CTGAAGGGCCGGGATCTTCTGACCCTGAAGAACTTTACTGGCGAAGAGATCAA
GTACATGCTCTGGCTCTCCGCGGACTTGAAGTTCCGCATTAAGCAGAAGGGGGA
ATACCTTCCGCTGCTTCAAGGAAAGAGCCTCGGCATGATCTTTGAGAAGCGCTC
AACCAGGACCCGCCTTTCTACTGAAACTGGGTTCGCGCTGCTCGGTGGCCACCC
CTGCTTCCTGACGACCCAGGACATCCACCTCGGAGTGAACGAATCCCTCACCGA
TACCGCCCGGGTGTTATCGAGCATGGCAGATGCCGTGCTGGCCAGGGTGTACAA
ACAGTCCGATCTGGACACTCTGGCCAAGGAGGCGTCAATTCCTATTATCAACGG
CCTTAGTGACCTCTACCATCCGATTCAGATCCTGGCCGATTACCTCACCCTGCAA
GAACACTACAGCTCCCTGAAGGGTCTGACATTGTCCTGGATCGGCGACGGCAAC
AACATTCTCCATTCCATCATGATGTCCGCCGCAAAATTCGGCATGCATCTTCAA
GCCGCCACGCCGAAGGGTTACGAGCCCGACGCTTCCGTGACTAAGCTCGCCGA
GCAGTACGCTAAGGAGAACGGAACCAAGCTTCTGCTGACTAACGACCCACTAG
AAGCAGCCCACGGGGGCAACGTGCTTATTACTGACACCTGGATCTCCATGGGCC
AGGAAGAAGAGAAAAAGAAGCGGCTGCAGGCGTTCCAGGGATATCAGGTCAC
CATGAAAACCGCCAAGGTCGCTGCCTCCGACTGGACCTTCCTGCACTGCCTGCC
TCGCAAGCCTGAAGAAGTGGACGACGAGGTGTTCTACTCGCCACGGAGCCTCG
TGTTCCCCGAGGCCGAGAATAGAAAGTGGACCATCATGGCCGTGATGGTGTCA
CTGCTCACCGACTACAGCCCGCAGCTTCAGAAGCCCAAGTTCTAGATAAGTGAA
>pARM1794
(SEQ ID NO: 170)
ATGCTGTTCAACCTGCGCATCCTGCTGAACAACGCCGCCTTCCGCAACGGCCAC
AACTTCATGGTGCGCAACTTCCGCTGCGGCCAGCCCCTGCAGAACAAGGTGCAG
CTGAAGGGCCGCGACCTGCTGACCCTGAAGAACTTCACCGGCGAGGAGATCAA
GTACATGCTGTGGCTGAGCGCCGACCTGAAGTTCCGCATCAAGCAGAAGGGCG
AGTACCTGCCCCTGCTGCAGGGCAAGAGCCTGGGCATGATCTTCGAGAAGCGC
AGCACCCGCACCCGCCTGAGCACCGAGACAGGCCTGGCCCTGCTGGGCGGCCA
CCCCTGCTTCCTGACCACCCAGGACATCCACCTGGGCGTGAACGAGAGCCTGAC
CGACACCGCCCGCGTGCTGAGCAGCATGGCCGACGCCGTGCTGGCCCGCGTGT
ACAAGCAGAGCGACCTGGACACCCTGGCCAAGGAGGCCAGCATCCCCATCATC
AACGGCCTGAGCGACCTGTACCACCCCATCCAGATCCTGGCCGACTACCTGACC
CTGCAGGAGCACTACAGCAGCCTGAAGGGCCTGACCCTGAGCTGGATCGGCGA
CGGCAACAACATCCTGCACAGCATCATGATGAGCGCCGCCAAGTTCGGCATGC
ACCTGCAGGCCGCCACCCCCAAGGGCTACGAGCCCGACGCCAGCGTGACCAAG
CTGGCCGAGCAGTACGCCAAGGAGAACGGCACCAAGCTGCTGCTGACCAACGA
CCCCCTGGAGGCCGCCCACGGCGGCAACGTGCTGATCACCGACACCTGGATCA
GCATGGGCCAGGAGGAGGAGAAGAAGAAGCGCCTGCAGGCCTTCCAGGGCTAC
CAGGTGACCATGAAGACCGCCAAGGTGGCCGCCAGCGACTGGACCTTCCTGCA
CTGCCTGCCCCGCAAGCCCGAGGAGGTGGACGACGAGGTGTTCTACAGCCCCC
GCAGCCTGGTGTTCCCCGAGGCCGAGAACCGCAAGTGGACCATCATGGCCGTG
ATGGTGAGCCTGCTGACCGACTACAGCCCCCAGCTGCAGAAGCCCAAGTTCTGA
ATAAGTGA
>pARM1795
(SEQ ID NO: 171)
ATGCTTTTCAACCTGAGAATCCTCTTGAACAATGCTGCTTTTCGGAATGGCCAC
AACTTTATGGTTCGGAACTTCCGTTGCGGCCAGCCTTTACAAAACAAGGTCCAG
CTGAAGGGCCGGGATTTGCTCACACTAAAGAACTTTACTGGAGAAGAGATCAA
GTACATGCTATGGCTGTCGGCCGACCTGAAGTTCCGTATCAAGCAGAAGGGAG
AATACCTTCCGCTGCTTCAAGGAAAGAGCCTCGGCATGATCTTTGAGAAGCGCT
CAACCAGGACCCGCCTTTCTACTGAAACTGGGTTCGCGCTGCTCGGTGGCCACC
CCTGCTTCCTGACGACCCAGGACATCCACCTCGGAGTGAACGAATCCCTCACCG
ATACCGCCCGGGTGTTATCGAGCATGGCAGATGCCGTGCTGGCCAGGGTGTACA
AACAGTCCGATCTCGATACCTTGGCAAAGGAGGCTTCCATTCCCATCATCAACG
GCCTGAGCGACCTGTACCACCCAATCCAAATCCTGGCTGACTACCTGACCCTGC
AAGAGCACTACAGCAGCCTGAAGGGTCTGACCCTGTCATGGATTGGCGATGGA
AACAATATTCTGCACTCCATCATGATGTCCGCCGCGAAGTTCGGAATGCATCTG
CAAGCCGCCACTCCAAAAGGATACGAACCGGATGCATCCGTGACCAAGTTGGC
GGAACAGTACGCGAAGGAGAACGGAACCAAGCTCCTGCTGACTAACGACCCGC
TCGAGGCTGCGCATGGGGGTAACGTGCTGATTACGGACACCTGGATCTCCATGG
GGCAGGAGGAAGAGAAGAAGAAGAGACTGCAGGCATTCCAGGGGTACCAGGT
CACCATGAAAACCGCAAAAGTGGCAGCTTCGGACTGGACTTTCCTGCATTGCCT
GCCGAGGAAGCCGGAGGAAGTCGACGACGAAGTGTTCTACTCGCCTCGGTCCC
TGGTGTTCCCCGAGGCCGAAAACCGGAAGTGGACCATCATGGCCGTGATGGTG
TCCTTGCTGACTGACTATAGCCCGCAGCTGCAGAAGCCTAAGTTCTAGATAAGT
GA
>pARM1796
(SEQ ID NO: 172)
ATGCTTTTCAATCTCCGCATCCTCCTTAACAACGCCGCGTTTAGAAACGGCCAC
AACTTCATGGTCCGGAACTTCAGATGTGGCCAGCCGCTTCAAAACAAGGTCCAG
CTGAAGGGCCGGGATCTTCTGACCCTGAAGAACTTTACTGGCGAAGAGATCAA
GTACATGCTCTGGCTCTCCGCGGACTTGAAGTTCCGCATTAAGCAGAAGGGGGA
ATACCTTCCGCTGCTTCAAGGAAAGAGCCTCGGCATGATCTTTGAGAAGCGCTC
AACCAGGACCCGCCTTTCTACTGAAACTGGGTTCGCGCTGCTCGGTGGCCACCC
CTGCTTCCTGACGACCCAGGACATCCACCTCGGAGTGAACGAATCCCTCACCGA
TACCGCCCGGGTGTTATCGAGCATGGCAGATGCCGTGCTGGCCAGGGTGTACAA
ACAGTCCGATCTGGACACTCTGGCCAAGGAGGCGTCAATTCCTATTATCAACGG
CCTTAGTGACCTCTACCATCCGATTCAGATCCTGGCCGATTACCTCACCCTGCAA
GAACACTACAGCTCCCTGAAGGGTCTGACATTGTCCTGGATCGGCGACGGCAAC
AACATTCTCCATTCCATCATGATGTCCGCCGCAAAATTCGGCATGCATCTTCAA
GCCGCCACGCCGAAGGGTTACGAGCCCGACGCTTCCGTGACTAAGCTCGCCGA
GCAGTACGCTAAGGAGAACGGAACCAAGCTTCTGCTGACTAACGACCCACTAG
AAGCAGCCCACGGGGGCAACGTGCTTATTACTGACACCTGGATCTCCATGGGCC
AGGAAGAAGAGAAAAAGAAGCGGCTGCAGGCGTTCCAGGGATATCAGGTCAC
CATGAAAACCGCCAAGGTCGCTGCCTCCGACTGGACCTTCCTGCACTGCCTGCC
TCGCAAGCCTGAAGAAGTGGACGACGAGGTGTTCTACTCGCCACGGAGCCTCG
TGTTCCCCGAGGCCGAGAATAGAAAGTGGACCATCATGGCCGTGATGGTGTCA
CTGCTCACCGACTACAGCCCGCAGCTTCAGAAGCCCAAGTTCTAGATAAGTGAA
>pARM1797
(SEQ ID NO: 173)
ATGCTGTTCAACCTGCGCATCCTGCTGAACAACGCCGCCTTCCGCAACGGCCAC
AACTTCATGGTGCGCAACTTCCGCTGCGGCCAGCCCCTGCAGAACAAGGTGCAG
CTGAAGGGCCGCGACCTGCTGACCCTGAAGAACTTCACCGGCGAGGAGATCAA
GTACATGCTGTGGCTGAGCGCCGACCTGAAGTTCCGCATCAAGCAGAAGGGCG
AGTACCTGCCCCTGCTGCAGGGCAAGAGCCTGGGCATGATCTTCGAGAAGCGC
AGCACCCGCACCCGCCTGAGCACCGAGACAGGCCTGGCCCTGCTGGGCGGCCA
CCCCTGCTTCCTGACCACCCAGGACATCCACCTGGGCGTGAACGAGAGCCTGAC
CGACACCGCCCGCGTGCTGAGCAGCATGGCCGACGCCGTGCTGGCCCGCGTGT
ACAAGCAGAGCGACCTGGACACCCTGGCCAAGGAGGCCAGCATCCCCATCATC
AACGGCCTGAGCGACCTGTACCACCCCATCCAGATCCTGGCCGACTACCTGACC
CTGCAGGAGCACTACAGCAGCCTGAAGGGCCTGACCCTGAGCTGGATCGGCGA
CGGCAACAACATCCTGCACAGCATCATGATGAGCGCCGCCAAGTTCGGCATGC
ACCTGCAGGCCGCCACCCCCAAGGGCTACGAGCCCGACGCCAGCGTGACCAAG
CTGGCCGAGCAGTACGCCAAGGAGAACGGCACCAAGCTGCTGCTGACCAACGA
CCCCCTGGAGGCCGCCCACGGCGGCAACGTGCTGATCACCGACACCTGGATCA
GCATGGGCCAGGAGGAGGAGAAGAAGAAGCGCCTGCAGGCCTTCCAGGGCTAC
CAGGTGACCATGAAGACCGCCAAGGTGGCCGCCAGCGACTGGACCTTCCTGCA
CTGCCTGCCCCGCAAGCCCGAGGAGGTGGACGACGAGGTGTTCTACAGCCCCC
GCAGCCTGGTGTTCCCCGAGGCCGAGAACCGCAAGTGGACCATCATGGCCGTG
ATGGTGAGCCTGCTGACCGACTACAGCCCCCAGCTGCAGAAGCCCAAGTTCTGA
ATAAGTGA
>pARM1798
(SEQ ID NO: 174)
ATGCTTTTCAACCTGAGAATCCTCTTGAACAATGCTGCTTTTCGGAATGGCCAC
AACTTTATGGTTCGGAACTTCCGTTGCGGCCAGCCTTTACAAAACAAGGTCCAG
CTGAAGGGCCGGGATTTGCTCACACTAAAGAACTTTACTGGAGAAGAGATCAA
GTACATGCTATGGCTGTCGGCCGACCTGAAGTTCCGTATCAAGCAGAAGGGAG
AATACCTTCCGCTGCTTCAAGGAAAGAGCCTCGGCATGATCTTTGAGAAGCGCT
CAACCAGGACCCGCCTTTCTACTGAAACTGGGTTCGCGCTGCTCGGTGGCCACC
CCTGCTTCCTGACGACCCAGGACATCCACCTCGGAGTGAACGAATCCCTCACCG
ATACCGCCCGGGTGTTATCGAGCATGGCAGATGCCGTGCTGGCCAGGGTGTACA
AACAGTCCGATCTCGATACCTTGGCAAAGGAGGCTTCCATTCCCATCATCAACG
GCCTGAGCGACCTGTACCACCCAATCCAAATCCTGGCTGACTACCTGACCCTGC
AAGAGCACTACAGCAGCCTGAAGGGTCTGACCCTGTCATGGATTGGCGATGGA
AACAATATTCTGCACTCCATCATGATGTCCGCCGCGAAGTTCGGAATGCATCTG
CAAGCCGCCACTCCAAAAGGATACGAACCGGATGCATCCGTGACCAAGTTGGC
GGAACAGTACGCGAAGGAGAACGGAACCAAGCTCCTGCTGACTAACGACCCGC
TCGAGGCTGCGCATGGGGGTAACGTGCTGATTACGGACACCTGGATCTCCATGG
GGCAGGAGGAAGAGAAGAAGAAGAGACTGCAGGCATTCCAGGGGTACCAGGT
CACCATGAAAACCGCAAAAGTGGCAGCTTCGGACTGGACTTTCCTGCATTGCCT
GCCGAGGAAGCCGGAGGAAGTCGACGACGAAGTGTTCTACTCGCCTCGGTCCC
TGGTGTTCCCCGAGGCCGAAAACCGGAAGTGGACCATCATGGCCGTGATGGTG
TCCTTGCTGACTGACTATAGCCCGCAGCTGCAGAAGCCTAAGTTCTAGATAAGT
GA
>pARM1799
(SEQ ID NO: 175)
ATGCTGTTCAACCTGCGCATCCTGCTGAACAACGCCGCCTTCCGCAACGGCCAC
AACTTCATGGTGCGCAACTTCCGCTGCGGCCAGCCCCTGCAGAACAAGGTGCAG
CTGAAGGGCCGCGACCTGCTGACCCTGAAGAACTTCACCGGCGAGGAGATCAA
GTACATGCTGTGGCTGAGCGCCGACCTGAAGTTCCGCATCAAGCAGAAGGGCG
AGTACCTGCCCCTGCTGCAGGGCAAGAGCCTGGGCATGATCTTCGAGAAGCGC
AGCACCCGCACCCGCCTGAGCACCGAGACAGGCTTCGCCCTGCTGGGCGGCCA
CCCCTGCTTCCTGACCACCCAGGACATCCACCTGGGCGTGAACGAGAGCCTGAC
CGACACCGCCCGCGTGCTGAGCAGCATGGCCGACGCCGTGCTGGCCCGCGTGT
ACAAGCAGAGCGACCTGGACACCCTGGCCAAGGAGGCCAGCATCCCCATCATC
AACGGCCTGAGCGACCTGTACCACCCCATCCAGATCCTGGCCGACTACCTGACC
CTGCAGGAGCACTACAGCAGCCTGAAGGGCCTGACCCTGAGCTGGATCGGCGA
CGGCAACAACATCCTGCACAGCATCATGATGAGCGCCGCCAAGTTCGGCATGC
ACCTGCAGGCCGCCACCCCCAAGGGCTACGAGCCCGACGCCAGCGTGACCAAG
CTGGCCGAGCAGTACGCCAAGGAGAACGGCACCAAGCTGCTGCTGACCAACGA
CCCCCTGGAGGCCGCCCACGGCGGCAACGTGCTGATCACCGACACCTGGATCA
GCATGGGCCAGGAGGAGGAGAAGAAGAAGCGCCTGCAGGCCTTCCAGGGCTAC
CAGGTGACCATGAAGACCGCCAAGGTGGCCGCCAGCGACTGGACCTTCCTGCA
CTGCCTGCCCCGCAAGCCCGAGGAGGTGGACGACGAGGTGTTCTACAGCCCCC
GCAGCCTGGTGTTCCCCGAGGCCGAGAACCGCAAGTGGACCATCATGGCCGTG
ATGGTGAGCCTGCTGACCGACTACAGCCCCCAGCTGCAGAAGCCCAAGTTCTGA
>pARM1800
(SEQ ID NO: 176)
ATGCTGTTCAACCTGCGCATCCTGCTGAACAACGCCGCCTTCCGCAACGGCCAC
AACTTCATGGTGCGCAACTTCCGCTGCGGCCAGCCCCTGCAGAACAAGGTGCAG
CTGAAGGGCCGCGACCTGCTGACCCTGAAGAACTTCACCGGCGAGGAGATCAA
GTACATGCTGTGGCTGAGCGCCGACCTGAAGTTCCGCATCAAGCAGAAGGGCG
AGTACCTGCCCCTGCTGCAGGGCAAGAGCCTGGGCATGATCTTCGAGAAGCGC
AGCACCCGCACCCGCCTGAGCACCGAGACAGGCTTCGCCCTGCTGGGCGGCCA
CCCCTGCTTCCTGACCACCCAGGACATCCACCTGGGCGTGAACGAGAGCCTGAC
CGACACCGCCCGCGTGCTGAGCAGCATGGCCGACGCCGTGCTGGCCCGCGTGT
ACAAGCAGAGCGACCTGGACACCCTGGCCAAGGAGGCCAGCATCCCCATCATC
AACGGCCTGAGCGACCTGTACCACCCCATCCAGATCCTGGCCGACTACCTGACC
CTGCAGGAGCACTACAGCAGCCTGAAGGGCCTGACCCTGAGCTGGATCGGCGA
CGGCAACAACATCCTGCACAGCATCATGATGAGCGCCGCCAAGTTCGGCATGC
ACCTGCAGGCCGCCACCCCCAAGGGCTACGAGCCCGACGCCAGCGTGACCAAG
CTGGCCGAGCAGTACGCCAAGGAGAACGGCACCAAGCTGCTGCTGACCAACGA
CCCCCTGGAGGCCGCCCACGGCGGCAACGTGCTGATCACCGACACCTGGATCA
GCATGGGCCAGGAGGAGGAGAAGAAGAAGCGCCTGCAGGCCTTCCAGGGCTAC
CAGGTGACCATGAAGACCGCCAAGGTGGCCGCCAGCGACTGGACCTTCCTGCA
CTGCCTGCCCCGCAAGCCCGAGGAGGTGGACGACGAGGTGTTCTACAGCCCCC
GCAGCCTGGTGTTCCCCGAGGCCGAGAACCGCAAGTGGACCATCATGGCCGTG
ATGGTGAGCCTGCTGACCGACTACAGCCCCCAGCTGCAGAAGCCCAAGTTCTGA
>pARM1801
(SEQ ID NO: 177)
ATGCTGTTCAACCTGCGCATCCTGCTGAACAACGCCGCCTTCCGCAACGGCCAC
AACTTCATGGTGCGCAACTTCCGCTGCGGCCAGCCCCTGCAGAACAAGGTGCAG
CTGAAGGGCCGCGACCTGCTGACCCTGAAGAACTTCACCGGCGAGGAGATCAA
GTACATGCTGTGGCTGAGCGCCGACCTGAAGTTCCGCATCAAGCAGAAGGGCG
AGTACCTGCCCCTGCTGCAGGGCAAGAGCCTGGGCATGATCTTCGAGAAGCGC
AGCACCCGCACCCGCCTGAGCACCGAGACAGGCTTCGCCCTGCTGGGCGGCCA
CCCCTGCTTCCTGACCACCCAGGACATCCACCTGGGCGTGAACGAGAGCCTGAC
CGACACCGCCCGCGTGCTGAGCAGCATGGCCGACGCCGTGCTGGCCCGCGTGT
ACAAGCAGAGCGACCTGGACACCCTGGCCAAGGAGGCCAGCATCCCCATCATC
AACGGCCTGAGCGACCTGTACCACCCCATCCAGATCCTGGCCGACTACCTGACC
CTGCAGGAGCACTACAGCAGCCTGAAGGGCCTGACCCTGAGCTGGATCGGCGA
CGGCAACAACATCCTGCACAGCATCATGATGAGCGCCGCCAAGTTCGGCATGC
ACCTGCAGGCCGCCACCCCCAAGGGCTACGAGCCCGACGCCAGCGTGACCAAG
CTGGCCGAGCAGTACGCCAAGGAGAACGGCACCAAGCTGCTGCTGACCAACGA
CCCCCTGGAGGCCGCCCACGGCGGCAACGTGCTGATCACCGACACCTGGATCA
GCATGGGCCAGGAGGAGGAGAAGAAGAAGCGCCTGCAGGCCTTCCAGGGCTAC
CAGGTGACCATGAAGACCGCCAAGGTGGCCGCCAGCGACTGGACCTTCCTGCA
CTGCCTGCCCCGCAAGCCCGAGGAGGTGGACGACGAGGTGTTCTACAGCCCCC
GCAGCCTGGTGTTCCCCGAGGCCGAGAACCGCAAGTGGACCATCATGGCCGTG
ATGGTGAGCCTGCTGACCGACTACAGCCCCCAGCTGCAGAAGCCCAAGTTCTGA
>pARM1802
(SEQ ID NO: 178)
ATGCTGTTCAACCTGCGCATCCTGCTGAACAACGCCGCCTTCCGCAACGGCCAC
AACTTCATGGTGCGCAACTTCCGCTGCGGCCAGCCCCTGCAGAACAAGGTGCAG
CTGAAGGGCCGCGACCTGCTGACCCTGAAGAACTTCACCGGCGAGGAGATCAA
GTACATGCTGTGGCTGAGCGCCGACCTGAAGTTCCGCATCAAGCAGAAGGGCG
AGTACCTGCCCCTGCTGCAGGGCAAGAGCCTGGGCATGATCTTCGAGAAGCGC
AGCACCCGCACCCGCCTGAGCACCGAGACAGGCTTCGCCCTGCTGGGCGGCCA
CCCCTGCTTCCTGACCACCCAGGACATCCACCTGGGCGTGAACGAGAGCCTGAC
CGACACCGCCCGCGTGCTGAGCAGCATGGCCGACGCCGTGCTGGCCCGCGTGT
ACAAGCAGAGCGACCTGGACACCCTGGCCAAGGAGGCCAGCATCCCCATCATC
AACGGCCTGAGCGACCTGTACCACCCCATCCAGATCCTGGCCGACTACCTGACC
CTGCAGGAGCACTACAGCAGCCTGAAGGGCCTGACCCTGAGCTGGATCGGCGA
CGGCAACAACATCCTGCACAGCATCATGATGAGCGCCGCCAAGTTCGGCATGC
ACCTGCAGGCCGCCACCCCCAAGGGCTACGAGCCCGACGCCAGCGTGACCAAG
CTGGCCGAGCAGTACGCCAAGGAGAACGGCACCAAGCTGCTGCTGACCAACGA
CCCCCTGGAGGCCGCCCACGGCGGCAACGTGCTGATCACCGACACCTGGATCA
GCATGGGCCAGGAGGAGGAGAAGAAGAAGCGCCTGCAGGCCTTCCAGGGCTAC
CAGGTGACCATGAAGACCGCCAAGGTGGCCGCCAGCGACTGGACCTTCCTGCA
CTGCCTGCCCCGCAAGCCCGAGGAGGTGGACGACGAGGTGTTCTACAGCCCCC
GCAGCCTGGTGTTCCCCGAGGCCGAGAACCGCAAGTGGACCATCATGGCCGTG
ATGGTGAGCCTGCTGACCGACTACAGCCCCCAGCTGCAGAAGCCCAAGTTCTGA
ATAAGTGAA
>pARM1803
(SEQ ID NO: 179)
ATGGGCGTCTTCAACCTGCGGATCCTGCTGAACAACGCCGCCTTCCGGAACGGC
CACAACTTCATGGTCCGCAACTTCAGATGCGGCCAGCCCCTGCAGAACAAGGTG
CAGCTGAAGGGCCGGGACCTGCTGACCCTGAAGAACTTCACCGGCGAAGAGAT
CAAGTACATGCTGTGGCTGAGCGCCGACCTGAAGTTCCGGATCAAGCAGAAGG
GCGAGTACCTGCCCCTGCTGCAAGGCAAGAGCCTGGGCATGATCTTCGAGAAG
CGGAGCACCCGGACCCGGCTGAGCACCGAGACAGGCTTTGCCCTGCTGGGAGG
CCACCCCTGCTTTCTGACCACCCAGGACATCCACCTGGGCGTGAACGAGAGCCT
GACCGACACCGCCAGAGTGCTGAGCAGCATGGCCGACGCCGTGCTGGCCCGGG
TGTACAAGCAGAGCGACCTGGACACCCTGGCCAAAGAGGCCAGCATCCCCATC
ATCAACGGCCTGAGCGACCTGTACCACCCCATCCAGATCCTGGCCGACTACCTG
ACCCTGCAGGAACACTACAGCTCCCTGAAGGGCCTGACCCTGAGCTGGATCGG
CGACGGCAACAACATCCTGCACAGCATCATGATGAGCGCCGCCAAGTTCGGCA
TGCATCTGCAGGCCGCCACCCCCAAGGGCTACGAGCCTGATGCCAGCGTGACC
AAGCTGGCCGAGCAGTACGCCAAAGAGAACGGCACCAAGCTGCTGCTGACCAA
CGACCCCCTGGAAGCCGCCCACGGCGGCAACGTGCTGATCACCGACACCTGGA
TCAGCATGGGCCAGGAAGAGGAAAAGAAGAAGCGGCTGCAGGCCTTCCAGGG
CTACCAGGTCACAATGAAGACCGCCAAGGTGGCCGCCAGCGACTGGACCTTCC
TGCACTGCCTGCCCCGGAAGCCCGAAGAGGTGGACGACGAGGTGTTCTACAGC
CCCCGGTCCCTGGTGTTCCCCGAGGCCGAGAACCGGAAGTGGACCATTATGGCC
GTGATGGTGTCCCTGCTGACCGACTACTCCCCCCAGCTGCAGAAGCCCAAGTTC
TAGATAAGTGAA
>pARM1804
(SEQ ID NO: 180)
ATGGGCGTCTTCAACCTGCGGATCCTGCTGAACAACGCCGCCTTCCGGAACGGC
CACAACTTCATGGTCCGCAACTTCAGATGCGGCCAGCCCCTGCAGAACAGGGTG
CAGCTGAAGGGCCGGGACCTGCTGACCCTGAAGAACTTCACCGGCGAAGAGAT
CAGGTACATGCTGTGGCTGAGCGCCGACCTGAAGTTCCGGATCAAGCAGAAGG
GCGAGTACCTGCCCCTGCTGCAAGGCAAGAGCCTGGGCATGATCTTCGAGAAG
CGGAGCACCCGGACCCGGCTGAGCACCGAGACAGGCTTTGCCCTGCTGGGAGG
CCACCCCTGCTTTCTGACCACCCAGGACATCCACCTGGGCGTGAACGAGAGCCT
GACCGACACCGCCAGAGTGCTGAGCAGCATGGCCGACGCCGTGCTGGCCCGGG
TGTACAAGCAGAGCGACCTGGACACCCTGGCCAAAGAGGCCAGCATCCCCATC
ATCAACGGCCTGAGCGACCTGTACCACCCCATCCAGATCCTGGCCGACTACCTG
ACCCTGCAGGAACACTACAGCTCCCTGAAGGGCCTGACCCTGAGCTGGATCGG
CGACGGCAACAACATCCTGCACAGCATCATGATGAGCGCCGCCAAGTTCGGCA
TGCATCTGCAGGCCGCCACCCCCAAGGGCTACGAGCCTGATGCCAGCGTGACC
AAGCTGGCCGAGCAGTACGCCAAAGAGAACGGCACCAAGCTGCTGCTGACCAA
CGACCCCCTGGAAGCCGCCCACGGCGGCAACGTGCTGATCACCGACACCTGGA
TCAGCATGGGCCAGGAAGAGGAAAAGAAGAAGCGGCTGCAGGCCTTCCAGGG
CTACCAGGTCACAATGAAGACCGCCAAGGTGGCCGCCAGCGACTGGACCTTCC
TGCACTGCCTGCCCCGGAAGCCCGAAGAGGTGGACGACGAGGTGTTCTACAGC
CCCCGGTCCCTGGTGTTCCCCGAGGCCGAGAACCGGAAGTGGACCATTATGGCC
GTGATGGTGTCCCTGCTGACCGACTACTCCCCCCAGCTGCAGAAGCCCAAGTTC
TAGATAAGTGAA
>pARM1805
(SEQ ID NO: 181)
ATGCTGGTCTTCAACCTGCGGATCCTGCTGAACAACGCCGCCTTCCGGAACGGC
CACAACTTCATGGTCCGCAACTTCAGATGCGGCCAGCCCCTGCAGAACAGGGTG
CAGCTGAAGGGCCGGGACCTGCTGACCCTGAAGAACTTCACCGGCGAAGAGAT
CAGGTACATGCTGTGGCTGAGCGCCGACCTGAAGTTCCGGATCAAGCAGAAGG
GCGAGTACCTGCCCCTGCTGCAAGGCAAGAGCCTGGGCATGATCTTCGAGAAG
CGGAGCACCCGGACCCGGCTGAGCACCGAGACAGGCTTTGCCCTGCTGGGAGG
CCACCCCTGCTTTCTGACCACCCAGGACATCCACCTGGGCGTGAACGAGAGCCT
GACCGACACCGCCAGAGTGCTGAGCAGCATGGCCGACGCCGTGCTGGCCCGGG
TGTACAAGCAGAGCGACCTGGACACCCTGGCCAAAGAGGCCAGCATCCCCATC
ATCAACGGCCTGAGCGACCTGTACCACCCCATCCAGATCCTGGCCGACTACCTG
ACCCTGCAGGAACACTACAGCTCCCTGAAGGGCCTGACCCTGAGCTGGATCGG
CGACGGCAACAACATCCTGCACAGCATCATGATGAGCGCCGCCAAGTTCGGCA
TGCATCTGCAGGCCGCCACCCCCAAGGGCTACGAGCCTGATGCCAGCGTGACC
AAGCTGGCCGAGCAGTACGCCAAAGAGAACGGCACCAAGCTGCTGCTGACCAA
CGACCCCCTGGAAGCCGCCCACGGCGGCAACGTGCTGATCACCGACACCTGGA
TCAGCATGGGCCAGGAAGAGGAAAAGAAGAAGCGGCTGCAGGCCTTCCAGGG
CTACCAGGTCACAATGAAGACCGCCAAGGTGGCCGCCAGCGACTGGACCTTCC
TGCACTGCCTGCCCCGGAAGCCCGAAGAGGTGGACGACGAGGTGTTCTACAGC
CCCCGGTCCCTGGTGTTCCCCGAGGCCGAGAACCGGAAGTGGACCATTATGGCC
GTGATGGTGTCCCTGCTGACCGACTACTCCCCCCAGCTGCAGAAGCCCAAGTTC
TAGATAAGTGAA
>pARM1806
(SEQ ID NO: 182)
ATGCTGTTCAACCTGAGGATCCTGCTGAACAACGCAGCTTTCAGGAACGGCCAC
AACTTCATGGTGAGGAACTTCCGGTGCGGCCAGCCCCTGCAGAACAAGGTGCA
GCTGAAGGGCAGGGACCTGCTGACCCTGAAGAACTTCACCGGAGAGGAGATCA
AGTACATGCTGTGGCTGAGCGCAGACCTGAAGTTCAGGATCAAGCAGAAGGGA
GAGTACCTGCCCCTGCTGCAGGGGAAGTCCCTGGGCATGATCTTCGAGAAGAG
GAGTACCAGGACCAGGCTGAGCACCGAAACCGGCTTCGCCCTGCTGGGAGGAC
ACCCCTGCTTCCTGACCACCCAGGACATCCACCTGGGCGTGAACGAGAGTCTGA
CCGACACCGCCAGGGTGCTGTCTAGCATGGCCGACGCCGTGCTGGCCAGGGTGT
ACAAGCAGTCAGACCTGGACACCCTGGCTAAGGAGGCCAGCATCCCCATCATC
AACGGCCTGAGCGACCTGTACCACCCCATCCAGATCCTGGCTGACTACCTGACC
CTGCAGGAGCACTACAGCTCTCTGAAGGGCCTGACCCTGAGCTGGATCGGCGA
CGGGAACAACATCCTGCACAGCATCATGATGAGCGCCGCCAAGTTCGGCATGC
ACCTGCAGGCCGCTACCCCCAAGGGTTACGAGCCCGACGCCAGCGTGACCAAG
CTGGCAGAGCAGTACGCCAAGGAGAACGGCACCAAGCTGCTGCTGACCAACGA
CCCCCTGGAGGCCGCCCACGGAGGCAACGTGCTGATCACCGACACCTGGATCA
GCATGGGACAGGAGGAGGAGAAGAAGAAGCGGCTGCAGGCTTTCCAGGGTTAC
CAGGTGACCATGAAGACCGCCAAGGTGGCTGCCAGCGACTGGACCTTCCTGCA
CTGCCTGCCCAGGAAGCCCGAGGAGGTGGACGACGAGGTGTTCTACTCTCCCA
GGAGCCTGGTGTTCCCCGAGGCCGAGAACAGGAAGTGGACCATCATGGCTGTG
ATGGTGTCCCTGCTGACCGACTACAGCCCCCAGCTGCAGAAGCCCAAGTTCTGA
ATAAGTGAA
>pARM1808
(SEQ ID NO: 183)
ATGCTGTTCAACCTGAGGATCCTGCTGAACAACGCAGCTTTCAGGAACGGCCAC
AACTTCATGGTGAGGAACTTCCGGTGCGGCCAGCCCCTGCAGAACAAGGTGCA
GCTGAAGGGCAGGGACCTGCTGACCCTGAAGAACTTCACCGGAGAGGAGATCA
AGTACATGCTGTGGCTGAGCGCAGACCTGAAGTTCAGGATCAAGCAGAAGGGA
GAGTACCTGCCCCTGCTGCAGGGGAAGTCCCTGGGCATGATCTTCGAGAAGAG
GAGTACCAGGACCAGGCTGAGCACCGAAACCGGCTTCGCCCTGCTGGGAGGAC
ACCCCTGCTTCCTGACGACCCAGGACATCCACCTCGGAGTGAACGAATCCCTCA
CCGATACCGCCCGGGTGTTATCAAGCATGGCAGATGCCGTGCTGGCCAGGGTGT
ACAAACAGTCCGATCTGGACACTCTGGCTAAGGAGGCCAGCATCCCCATCATCA
ACGGCCTGAGCGACCTGTACCACCCCATCCAGATCCTGGCTGACTACCTGACCC
TGCAGGAGCACTACAGCTCTCTGAAGGGCCTGACCCTGAGCTGGATCGGCGAC
GGGAACAACATCCTGCACTCCATCATGATGTCCGCCGCGAAGTTCGGAATGCAT
CTGCAAGCCGCCACGCCAAAAGGATACGAACCGGATGCGCCCGTGACAAAGTT
GGCGGAACAGTACGCTAAGGAGAACGGAACCAAGCTGCTGCTGACCAACGACC
CCCTGGAGGCCGCCCACGGAGGCAACGTGCTGATCACCGACACCTGGATCAGC
ATGGGACAGGAGGAGGAGAAGAAGAAGCGGCTGCAGGCTTTCCAGGGTTACCA
GGTGACCATGAAGACCGCCAAGGTGGCTGCCAGCGACTGGACCTTCCTGCACT
GCCTGCCCAGGAAGCCCGAGGAGGTGGACGACGAGGTGTTCTACTCTCCCAGG
AGCCTGGTGTTCCCCGAGGCCGAGAACAGGAAGTGGACCATCATGGCTGTGAT
GGTGTCCCTGCTGACCGACTACAGCCCCCAGCTGCAGAAGCCCAAGTTCTGAAT
AAGTGAA
>pARM1809
(SEQ ID NO: 184)
ATGCTGTTCAACCTGCGCATCCTGCTGAACAACGCCGCCTTCCGCAACGGCCAC
AACTTCATGGTGCGCAACTTCCGCTGCGGCCAGCCCCTGCAGAACAAGGTGCAG
CTGAAGGGCCGCGACCTGCTGACCCTGAAGAACTTCACCGGCGAGGAGATCAA
GTACATGCTGTGGCTGAGCGCCGACCTGAAGTTCCGCATCAAGCAGAAGGGCG
AGTACCTGCCCCTGCTGCAGGGCAAGAGCCTGGGCATGATCTTCGAGAAGCGC
AGCACCCGCACCCGCCTGAGCACCGAGACAGGCTTCGCCCTGCTGGGCGGCCA
CCCCTGCTTCCTGACCACCCAGGACATCCACCTGGGCGTGAACGAGAGCCTGAC
CGACACCGCCCGCGTGCTGAGCAGCATGGCCGACGCCGTGCTGGCCCGCGTGT
ACAAGCAGAGCGACCTGGACACCCTGGCCAAGGAGGCCAGCATCCCCATCATC
AACGGCCTGAGCGACCTGTACCACCCCATCCAGATCCTGGCCGACTACCTGACC
CTGCAGGAGCACTACAGCAGCCTGAAGGGCCTGACCCTGAGCTGGATCGGCGA
CGGCAACAACATCCTGCACAGCATCATGATGAGCGCCGCCAAGTTCGGCATGC
ACCTGCAGGCCGCCACCCCCAAGGGCTACGAGCCCGACGCCAGCGTGACCAAG
CTGGCCGAGCAGTACGCCAAGGAGAACGGCACCAAGCTGCTGCTGACCAACGA
CCCCCTGGAGGCCGCCCACGGCGGCAACGTGCTGATCACCGACACCTGGATCA
GCATGGGCCAGGAGGAGGAGAAGAAGAAGCGCCTGCAGGCCTTCCAGGGCTAC
CAGGTGACCATGAAGACCGCCAAGGTGGCCGCCAGCGACTGGACCTTCCTGCA
CTGCCTGCCCCGCAAGCCCGAGGAGGTGGACGACGAGGTGTTCTACAGCCCCC
GCAGCCTGGTGTTCCCCGAGGCCGAGAACCGCAAGTGGACCATCATGGCCGTG
ATGGTGAGCCTGCTGACCGACTACAGCCCCCAGCTGCAGAAGCCCAAGTTCTGA
ATAAGTGAA
>pARM1816
(SEQ ID NO: 185)
ATGCTGTTCAACCTGCGCATCCTGCTGAACAACGCCGCCTTCCGCAACGGCCAC
AACTTCATGGTGCGCAACTTCCGCTGCGGCCAGCCCCTGCAGAACAAGGTGCAG
CTGAAGGGCCGCGACCTGCTGACCCTGAAGAACTTCACCGGCGAGGAGATCAA
GTACATGCTGTGGCTGAGCGCCGACCTGAAGTTCCGCATCAAGCAGAAGGGCG
AGTACCTGCCCCTGCTGCAGGGCAAGAGCCTGGGCATGATCTTCGAGAAGCGC
AGCACCCGCACCCGCCTGAGCACCGAGACAGGCTTCGCCCTGCTGGGCGGCCA
CCCCTGCTTCCTGACCACCCAGGACATCCACCTGGGCGTGAACGAGAGCCTGAC
CGACACCGCCCGCGTGCTGAGCAGCATGGCCGACGCCGTGCTGGCCCGCGTGT
ACAAGCAGAGCGACCTGGACACCCTGGCCAAGGAGGCCAGCATCCCCATCATC
AACGGCCTGAGCGACCTGTACCACCCCATCCAGATCCTGGCCGACTACCTGACC
CTGCAGGAGCACTACAGCAGCCTGAAGGGCCTGACCCTGAGCTGGATCGGCGA
CGGCAACAACATCCTGCACAGCATCATGATGAGCGCCGCCAAGTTCGGCATGC
ACCTGCAGGCCGCCACCCCCAAGGGCTACGAGCCCGACGCCAGCGTGACCAAG
CTGGCCGAGCAGTACGCCAAGGAGAACGGCACCAAGCTGCTGCTGACCAACGA
CCCCCTGGAGGCCGCCCACGGCGGCAACGTGCTGATCACCGACACCTGGATCA
GCATGGGCCAGGAGGAGGAGAAGAAGAAGCGCCTGCAGGCCTTCCAGGGCTAC
CAGGTGACCATGAAGACCGCCAAGGTGGCCGCCAGCGACTGGACCTTCCTGCA
CTGCCTGCCCCGCAAGCCCGAGGAGGTGGACGACGAGGTGTTCTACAGCCCCC
GCAGCCTGGTGTTCCCCGAGGCCGAGAACCGCAAGTGGACCATCATGGCCGTG
ATGGTGAGCCTGCTGACCGACTACAGCCCCCAGCTGCAGAAGCCCAAGTTCTGA
ATAAGTGAA
>pARM1822
(SEQ ID NO: 186)
ATGCTTTTCAACTTGAGAATCCTGCTGAACAACGCCGCCTTTCGCAACGGTCAC
AATTTTATGGTCAGAAACTTCAGATGCGGACAGCCCCTCCAAAACAAGGTCCAG
CTGAAGGGCCGCGATCTCCTCACCCTGAAGAACTTCACGGGGGAGGAGATCAA
GTACATGCTGTGGCTCTCCGCTGACCTGAAGTTCAGGATCAAGCAGAAGGGAG
AATATCTGCCGCTGCTGCAAGGGAAGTCCCTGGGGATGATTTTCGAGAAGCGG
AGCACCCGGACTCGGCTCTCCACTGAAACTGGTTTCGCCCTTCTGGGCGGTCAC
CCCTGCTTCCTGACCACTCAAGACATTCACCTCGGAGTGAACGAGTCCTTGACT
GACACCGCCCGGGTGCTGTCGAGCATGGCAGACGCCGTGCTAGCCCGCGTGTA
CAAGCAGTCAGACCTCGATACCCTGGCCAAGGAGGCTTCGATCCCGATCATCAA
CGGGTTGTCCGACCTGTACCACCCGATTCAGATTCTCGCCGACTACCTCACCCT
GCAAGAGCATTACAGCTCCCTGAAGGGGCTTACCCTGTCCTGGATTGGCGACGG
AAACAACATCCTGCACTCCATTATGATGTCGGCGGCCAAGTTCGGCATGCACCT
CCAAGCCGCGACCCCTAAGGGTTACGAACCAGACGCGTCAGTGACTAAGCTGG
CCGAACAGTACGCAAAGGAAAATGGCACGAAGCTGCTCCTGACCAACGATCCG
TTGGAAGCCGCCCATGGCGGAAATGTGCTCATCACCGACACCTGGATCTCGATG
GGACAGGAGGAAGAGAAGAAGAAGCGGCTGCAGGCGTTCCAGGGCTACCAGG
TCACCATGAAAACTGCCAAGGTGGCCGCCAGCGACTGGACCTTCCTGCACTGCC
TTCCGCGCAAGCCTGAGGAGGTGGACGATGAAGTGTTCTACTCTCCACGGTCCC
TGGTGTTCCCCGAGGCGGAGAACCGCAAATGGACCATCATGGCTGTGATGGTC
AGCCTGCTGACCGATTACAGCCCTCAGTTGCAAAAGCCGAAGTTTTGA
>pARM1823
(SEQ ID NO: 187)
ATGCTGTTCAACCTCCGCATCCTCCTCAACAACGCCGCATTCAGAAACGGGCAC
AACTTCATGGTCAGAAACTTCCGCTGCGGGCAACCCCTACAAAACAAGGTCCA
GCTCAAGGGGCGGGACCTCCTGACCCTGAAGAACTTCACCGGCGAAGAGATCA
AGTACATGCTGTGGCTCTCCGCCGACCTGAAGTTCCGCATCAAGCAGAAGGGA
GAGTACCTCCCGCTGCTGCAAGGGAAGTCGCTGGGGATGATCTTCGAGAAGCG
GTCAACCAGAACCCGGCTGTCAACCGAAACCGGGTTCGCACTGCTGGGGGGAC
ACCCGTGCTTCCTGACCACCCAAGACATCCACCTGGGAGTGAACGAATCGCTGA
CCGACACCGCCCGCGTGCTGAGCTCAATGGCGGACGCCGTGCTGGCCCGCGTGT
ACAAGCAGTCCGACCTGGACACCCTGGCCAAGGAAGCGTCCATCCCGATCATC
AACGGACTGTCCGACCTGTACCACCCGATCCAGATCCTGGCAGACTACCTGACC
CTGCAAGAACACTACAGCTCCCTGAAGGGCCTGACCCTGTCATGGATCGGGGA
CGGGAACAACATCCTGCACTCCATAATGATGTCAGCCGCCAAGTTCGGAATGCA
CCTCCAAGCCGCAACCCCGAAGGGCTACGAACCGGACGCATCAGTGACCAAAC
TGGCCGAGCAGTACGCCAAGGAAAACGGCACCAAGCTCCTGCTGACCAACGAC
CCGCTGGAGGCCGCACACGGGGGGAACGTGCTGATCACCGACACCTGGATCTC
CATGGGACAGGAGGAGGAAAAGAAGAAGCGGCTGCAGGCGTTCCAGGGGTAC
CAGGTCACCATGAAAACCGCGAAGGTCGCGGCATCAGACTGGACCTTCCTGCA
CTGCCTGCCCCGGAAGCCGGAAGAGGTGGACGACGAGGTGTTCTACTCGCCGC
GCTCGCTGGTGTTCCCCGAGGCGGAGAACAGGAAGTGGACCATCATGGCGGTG
ATGGTCAGCCTCCTGACCGACTACTCGCCGCAGCTGCAGAAGCCGAAGTTCTGA
>pARM1840
(SEQ ID NO: 188)
ATGCTTTTCAATCTCCGCATCCTCCTTAACAACGCCGCGTTTAGAAACGGCCAC
AACTTCATGGTCCGGAACTTCAGATGTGGCCAGCCGCTTCAAAACAAGGTCCAG
CTGAAGGGCCGGGATCTTCTGACCCTGAAGAACTTTACTGGCGAAGAGATCAA
GTACATGCTCTGGCTCTCCGCGGACTTGAAGTTCCGCATTAAGCAGAAGGGGGA
ATACCTTCCGCTGCTTCAAGGAAAGAGCCTCGGCATGATCTTTGAGAAGCGCTC
AACCAGGACCCGCCTTTCTACTGAAACTGGGTTCGCGCTGCTCGGTGGCCACCC
CTGCTTCCTGACGACCCAGGACATCCACCTCGGAGTGAACGAATCCCTCACCGA
TACCGCCCGGGTGTTATCGAGCATGGCAGATGCCGTGCTGGCCAGGGTGTACAA
ACAGTCCGATCTGGACACTCTGGCCAAGGAGGCGTCAATTCCTATTATCAACGG
CCTTAGTGACCTCTACCATCCGATTCAGATCCTGGCCGATTACCTCACCCTGCAA
GAACACTACAGCTCCCTGAAGGGTCTGACATTGTCCTGGATCGGCGACGGCAAC
AACATTCTCCATTCCATCATGATGTCCGCCGCAAAATTCGGCATGCATCTTCAA
GCCGCCACGCCGAAGGGTTACGAGCCCGACGCTTCCGTGACTAAGCTCGCCGA
GCAGTACGCTAAGGAGAACGGAACCAAGCTTCTGCTGACTAACGACCCACTAG
AAGCAGCCCACGGGGGCAACGTGCTTATTACTGACACCTGGATCTCCATGGGCC
AGGAAGAAGAGAAAAAGAAGCGGCTGCAGGCGTTCCAGGGATATCAGGTCAC
CATGAAAACCGCCAAGGTCGCTGCCTCCGACTGGACCTTCCTGCACTGCCTGCC
TCGCAAGCCTGAAGAAGTGGACGACGAGGTGTTCTACTCGCCACGGAGCCTCG
TGTTCCCCGAGGCCGAGAATAGAAAGTGGACCATCATGGCCGTGATGGTGTCA
CTGCTCACCGACTACAGCCCGCAGCTTCAGAAGCCCAAGTTCTGAATAAGTAGA
>pARM1841
(SEQ ID NO: 189)
ATGCTTTTCAACCTGAGAATCCTCTTGAACAATGCTGCTTTTCGGAATGGCCAC
AACTTTATGGTTCGGAACTTCCGTTGCGGCCAGCCTTTACAAAACAAGGTCCAG
CTGAAGGGCCGGGATTTGCTCACACTAAAGAACTTTACTGGAGAAGAGATCAA
GTACATGCTATGGCTGTCGGCCGACCTGAAGTTCCGTATCAAGCAGAAGGGAG
AATACCTTCCGCTGCTTCAAGGAAAGAGCCTCGGCATGATCTTTGAGAAGCGCT
CAACCAGGACCCGCCTTTCTACTGAAACTGGGTTCGCGCTGCTCGGTGGCCACC
CCTGCTTCCTGACGACCCAGGACATCCACCTCGGAGTGAACGAATCCCTCACCG
ATACCGCCCGGGTGTTATCGAGCATGGCAGATGCCGTGCTGGCCAGGGTGTACA
AACAGTCCGATCTCGATACCTTGGCAAAGGAGGCTTCCATTCCCATCATCAACG
GCCTGAGCGACCTGTACCACCCAATCCAAATCCTGGCTGACTACCTGACCCTGC
AAGAGCACTACAGCAGCCTGAAGGGTCTGACCCTGTCATGGATTGGCGATGGA
AACAATATTCTGCACTCCATCATGATGTCCGCCGCGAAGTTCGGAATGCATCTG
CAAGCCGCCACTCCAAAAGGATACGAACCGGATGCATCCGTGACCAAGTTGGC
GGAACAGTACGCGAAGGAGAACGGAACCAAGCTCCTGCTGACTAACGACCCGC
TCGAGGCTGCGCATGGGGGTAACGTGCTGATTACGGACACCTGGATCTCCATGG
GGCAGGAGGAAGAGAAGAAGAAGAGACTGCAGGCATTCCAGGGGTACCAGGT
CACCATGAAAACCGCAAAAGTGGCAGCTTCGGACTGGACTTTCCTGCATTGCCT
GCCGAGGAAGCCGGAGGAAGTCGACGACGAAGTGTTCTACTCGCCTCGGTCCC
TGGTGTTCCCCGAGGCCGAAAACCGGAAGTGGACCATCATGGCCGTGATGGTG
TCCTTGCTGACTGACTATAGCCCGCAGCTGCAGAAGCCTAAGTTCTGAATAAGT
AGA
>pARM1842
(SEQ ID NO: 190)
ATGCTGTTCAACCTGCGCATCCTGCTGAACAACGCCGCCTTCCGCAACGGCCAC
AACTTCATGGTGCGCAACTTCCGCTGCGGCCAGCCCCTGCAGAACAAGGTGCAG
CTGAAGGGCCGCGACCTGCTGACCCTGAAGAACTTCACCGGCGAGGAGATCAA
GTACATGCTGTGGCTGAGCGCCGACCTGAAGTTCCGCATCAAGCAGAAGGGCG
AGTACCTGCCCCTGCTGCAGGGCAAGAGCCTGGGCATGATCTTCGAGAAGCGC
AGCACCCGCACCCGCCTGAGCACCGAGACAGGCCTGGCCCTGCTGGGCGGCCA
CCCCTGCTTCCTGACCACCCAGGACATCCACCTGGGCGTGAACGAGAGCCTGAC
CGACACCGCCCGCGTGCTGAGCAGCATGGCCGACGCCGTGCTGGCCCGCGTGT
ACAAGCAGAGCGACCTGGACACCCTGGCCAAGGAGGCCAGCATCCCCATCATC
AACGGCCTGAGCGACCTGTACCACCCCATCCAGATCCTGGCCGACTACCTGACC
CTGCAGGAGCACTACAGCAGCCTGAAGGGCCTGACCCTGAGCTGGATCGGCGA
CGGCAACAACATCCTGCACAGCATCATGATGAGCGCCGCCAAGTTCGGCATGC
ACCTGCAGGCCGCCACCCCCAAGGGCTACGAGCCCGACGCCAGCGTGACCAAG
CTGGCCGAGCAGTACGCCAAGGAGAACGGCACCAAGCTGCTGCTGACCAACGA
CCCCCTGGAGGCCGCCCACGGCGGCAACGTGCTGATCACCGACACCTGGATCA
GCATGGGCCAGGAGGAGGAGAAGAAGAAGCGCCTGCAGGCCTTCCAGGGCTAC
CAGGTGACCATGAAGACCGCCAAGGTGGCCGCCAGCGACTGGACCTTCCTGCA
CTGCCTGCCCCGCAAGCCCGAGGAGGTGGACGACGAGGTGTTCTACAGCCCCC
GCAGCCTGGTGTTCCCCGAGGCCGAGAACCGCAAGTGGACCATCATGGCCGTG
ATGGTGAGCCTGCTGACCGACTACAGCCCCCAGCTGCAGAAGCCCAAGTTCTGA
ATAAGTAGA
>pARM1843
(SEQ ID NO: 191)
ATGCTTTTCAATCTCCGCATCCTCCTTAACAACGCCGCGTTTAGAAACGGCCAC
AACTTCATGGTCCGGAACTTCAGATGTGGCCAGCCGCTTCAAAACAAGGTCCAG
CTGAAGGGCCGGGATCTTCTGACCCTGAAGAACTTTACTGGCGAAGAGATCAA
GTACATGCTCTGGCTCTCCGCGGACTTGAAGTTCCGCATTAAGCAGAAGGGGGA
ATACCTTCCGCTGCTTCAAGGAAAGAGCCTCGGCATGATCTTTGAGAAGCGCTC
AACCAGGACCCGCCTTTCTACTGAAACTGGGTTCGCGCTGCTCGGTGGCCACCC
CTGCTTCCTGACGACCCAGGACATCCACCTCGGAGTGAACGAATCCCTCACCGA
TACCGCCCGGGTGTTATCGAGCATGGCAGATGCCGTGCTGGCCAGGGTGTACAA
ACAGTCCGATCTGGACACTCTGGCCAAGGAGGCGTCAATTCCTATTATCAACGG
CCTTAGTGACCTCTACCATCCGATTCAGATCCTGGCCGATTACCTCACCCTGCAA
GAACACTACAGCTCCCTGAAGGGTCTGACATTGTCCTGGATCGGCGACGGCAAC
AACATTCTCCATTCCATCATGATGTCCGCCGCAAAATTCGGCATGCATCTTCAA
GCCGCCACGCCGAAGGGTTACGAGCCCGACGCTTCCGTGACTAAGCTCGCCGA
GCAGTACGCTAAGGAGAACGGAACCAAGCTTCTGCTGACTAACGACCCACTAG
AAGCAGCCCACGGGGGCAACGTGCTTATTACTGACACCTGGATCTCCATGGGCC
AGGAAGAAGAGAAAAAGAAGCGGCTGCAGGCGTTCCAGGGATATCAGGTCAC
CATGAAAACCGCCAAGGTCGCTGCCTCCGACTGGACCTTCCTGCACTGCCTGCC
TCGCAAGCCTGAAGAAGTGGACGACGAGGTGTTCTACTCGCCACGGAGCCTCG
TGTTCCCCGAGGCCGAGAATAGAAAGTGGACCATCATGGCCGTGATGGTGTCA
CTGCTCACCGACTACAGCCCGCAGCTTCAGAAGCCCAAGTTCTAG
>pARM1844
(SEQ ID NO: 192)
ATGCTTTTCAATCTCCGCATCCTCCTTAACAACGCCGCGTTTAGAAACGGCCAC
AACTTCATGGTCCGGAACTTCAGATGTGGCCAGCCGCTTCAAAACAAGGTCCAG
CTGAAGGGCCGGGATCTTCTGACCCTGAAGAACTTTACTGGCGAAGAGATCAA
GTACATGCTCTGGCTCTCCGCGGACTTGAAGTTCCGCATTAAGCAGAAGGGGGA
ATACCTTCCGCTGCTTCAAGGAAAGAGCCTCGGCATGATCTTTGAGAAGCGCTC
AACCAGGACCCGCCTTTCTACTGAAACTGGGTTCGCGCTGCTCGGTGGCCACCC
CTGCTTCCTGACGACCCAGGACATCCACCTCGGAGTGAACGAATCCCTCACCGA
TACCGCCCGGGTGTTATCGAGCATGGCAGATGCCGTGCTGGCCAGGGTGTACAA
ACAGTCCGATCTGGACACTCTGGCCAAGGAGGCGTCAATTCCTATTATCAACGG
CCTTAGTGACCTCTACCATCCGATTCAGATCCTGGCCGATTACCTCACCCTGCAA
GAACACTACAGCTCCCTGAAGGGTCTGACATTGTCCTGGATCGGCGACGGCAAC
AACATTCTCCATTCCATCATGATGTCCGCCGCAAAATTCGGCATGCATCTTCAA
GCCGCCACGCCGAAGGGTTACGAGCCCGACGCTTCCGTGACTAAGCTCGCCGA
GCAGTACGCTAAGGAGAACGGAACCAAGCTTCTGCTGACTAACGACCCACTAG
AAGCAGCCCACGGGGGCAACGTGCTTATTACTGACACCTGGATCTCCATGGGCC
AGGAAGAAGAGAAAAAGAAGCGGCTGCAGGCGTTCCAGGGATATCAGGTCAC
CATGAAAACCGCCAAGGTCGCTGCCTCCGACTGGACCTTCCTGCACTGCCTGCC
TCGCAAGCCTGAAGAAGTGGACGACGAGGTGTTCTACTCGCCACGGAGCCTCG
TGTTCCCCGAGGCCGAGAATAGAAAGTGGACCATCATGGCCGTGATGGTGTCA
CTGCTCACCGACTACAGCCCGCAGCTTCAGAAGCCCAAGTTCTAG
>pARM1845
(SEQ ID NO: 193)
ATGCTTTTCAATCTCCGCATCCTCCTTAACAACGCCGCGTTTAGAAACGGCCAC
AACTTCATGGTCCGGAACTTCAGATGTGGCCAGCCGCTTCAAAACAAGGTCCAG
CTGAAGGGCCGGGATCTTCTGACCCTGAAGAACTTTACTGGCGAAGAGATCAA
GTACATGCTCTGGCTCTCCGCGGACTTGAAGTTCCGCATTAAGCAGAAGGGGGA
ATACCTTCCGCTGCTTCAAGGAAAGAGCCTCGGCATGATCTTTGAGAAGCGCTC
AACCAGGACCCGCCTTTCTACTGAAACTGGGTTCGCGCTGCTCGGTGGCCACCC
CTGCTTCCTGACGACCCAGGACATCCACCTCGGAGTGAACGAATCCCTCACCGA
TACCGCCCGGGTGTTATCGAGCATGGCAGATGCCGTGCTGGCCAGGGTGTACAA
ACAGTCCGATCTGGACACTCTGGCCAAGGAGGCGTCAATTCCTATTATCAACGG
CCTTAGTGACCTCTACCATCCGATTCAGATCCTGGCCGATTACCTCACCCTGCAA
GAACACTACAGCTCCCTGAAGGGTCTGACATTGTCCTGGATCGGCGACGGCAAC
AACATTCTCCATTCCATCATGATGTCCGCCGCAAAATTCGGCATGCATCTTCAA
GCCGCCACGCCGAAGGGTTACGAGCCCGACGCTTCCGTGACTAAGCTCGCCGA
GCAGTACGCTAAGGAGAACGGAACCAAGCTTCTGCTGACTAACGACCCACTAG
AAGCAGCCCACGGGGGCAACGTGCTTATTACTGACACCTGGATCTCCATGGGCC
AGGAAGAAGAGAAAAAGAAGCGGCTGCAGGCGTTCCAGGGATATCAGGTCAC
CATGAAAACCGCCAAGGTCGCTGCCTCCGACTGGACCTTCCTGCACTGCCTGCC
TCGCAAGCCTGAAGAAGTGGACGACGAGGTGTTCTACTCGCCACGGAGCCTCG
TGTTCCCCGAGGCCGAGAATAGAAAGTGGACCATCATGGCCGTGATGGTGTCA
CTGCTCACCGACTACAGCCCGCAGCTTCAGAAGCCCAAGTTCTAG
>pARM1846
(SEQ ID NO: 194)
ATGCTTTTCAATCTCCGCATCCTCCTTAACAACGCCGCGTTTAGAAACGGCCAC
AACTTCATGGTCCGGAACTTCAGATGTGGCCAGCCGCTTCAAAACAAGGTCCAG
CTGAAGGGCCGGGATCTTCTGACCCTGAAGAACTTTACTGGCGAAGAGATCAA
GTACATGCTCTGGCTCTCCGCGGACTTGAAGTTCCGCATTAAGCAGAAGGGGGA
ATACCTTCCGCTGCTTCAAGGAAAGAGCCTCGGCATGATCTTTGAGAAGCGCTC
AACCAGGACCCGCCTTTCTACTGAAACTGGGTTCGCGCTGCTCGGTGGCCACCC
CTGCTTCCTGACGACCCAGGACATCCACCTCGGAGTGAACGAATCCCTCACCGA
TACCGCCCGGGTGTTATCGAGCATGGCAGATGCCGTGCTGGCCAGGGTGTACAA
ACAGTCCGATCTGGACACTCTGGCCAAGGAGGCGTCAATTCCTATTATCAACGG
CCTTAGTGACCTCTACCATCCGATTCAGATCCTGGCCGATTACCTCACCCTGCAA
GAACACTACAGCTCCCTGAAGGGTCTGACATTGTCCTGGATCGGCGACGGCAAC
AACATTCTCCATTCCATCATGATGTCCGCCGCAAAATTCGGCATGCATCTTCAA
GCCGCCACGCCGAAGGGTTACGAGCCCGACGCTTCCGTGACTAAGCTCGCCGA
GCAGTACGCTAAGGAGAACGGAACCAAGCTTCTGCTGACTAACGACCCACTAG
AAGCAGCCCACGGGGGCAACGTGCTTATTACTGACACCTGGATCTCCATGGGCC
AGGAAGAAGAGAAAAAGAAGCGGCTGCAGGCGTTCCAGGGATATCAGGTCAC
CATGAAAACCGCCAAGGTCGCTGCCTCCGACTGGACCTTCCTGCACTGCCTGCC
TCGCAAGCCTGAAGAAGTGGACGACGAGGTGTTCTACTCGCCACGGAGCCTCG
TGTTCCCCGAGGCCGAGAATAGAAAGTGGACCATCATGGCCGTGATGGTGTCA
CTGCTCACCGACTACAGCCCGCAGCTTCAGAAGCCCAAGTTCTAG
>pARM1847
(SEQ ID NO: 195)
ATGCTTTTCAATCTCCGCATCCTCCTTAACAACGCCGCGTTTAGAAACGGCCAC
AACTTCATGGTCCGGAACTTCAGATGTGGCCAGCCGCTTCAAAACAAGGTCCAG
CTGAAGGGCCGGGATCTTCTGACCCTGAAGAACTTTACTGGCGAAGAGATCAA
GTACATGCTCTGGCTCTCCGCGGACTTGAAGTTCCGCATTAAGCAGAAGGGGGA
ATACCTTCCGCTGCTTCAAGGAAAGAGCCTCGGCATGATCTTTGAGAAGCGCTC
AACCAGGACCCGCCTTTCTACTGAAACTGGGTTCGCGCTGCTCGGTGGCCACCC
CTGCTTCCTGACGACCCAGGACATCCACCTCGGAGTGAACGAATCCCTCACCGA
TACCGCCCGGGTGTTATCGAGCATGGCAGATGCCGTGCTGGCCAGGGTGTACAA
ACAGTCCGATCTGGACACTCTGGCCAAGGAGGCGTCAATTCCTATTATCAACGG
CCTTAGTGACCTCTACCATCCGATTCAGATCCTGGCCGATTACCTCACCCTGCAA
GAACACTACAGCTCCCTGAAGGGTCTGACATTGTCCTGGATCGGCGACGGCAAC
AACATTCTCCATTCCATCATGATGTCCGCCGCAAAATTCGGCATGCATCTTCAA
GCCGCCACGCCGAAGGGTTACGAGCCCGACGCTTCCGTGACTAAGCTCGCCGA
GCAGTACGCTAAGGAGAACGGAACCAAGCTTCTGCTGACTAACGACCCACTAG
AAGCAGCCCACGGGGGCAACGTGCTTATTACTGACACCTGGATCTCCATGGGCC
AGGAAGAAGAGAAAAAGAAGCGGCTGCAGGCGTTCCAGGGATATCAGGTCAC
CATGAAAACCGCCAAGGTCGCTGCCTCCGACTGGACCTTCCTGCACTGCCTGCC
TCGCAAGCCTGAAGAAGTGGACGACGAGGTGTTCTACTCGCCACGGAGCCTCG
TGTTCCCCGAGGCCGAGAATAGAAAGTGGACCATCATGGCCGTGATGGTGTCA
CTGCTCACCGACTACAGCCCGCAGCTTCAGAAGCCCAAGTTCTAG
>pARM1882
(SEQ ID NO: 196)
ATGGTGTTCAACCTCCGCATCCTCCTCAACAACGCCGCATTCAGAAACGGGCAC
AACTTCATGGTCAGAAACTTCCGCTGCGGGCAACCCCTACAAAACAAGGTCCA
GCTCAAGGGGCGGGACCTCCTGACCCTGAAGAACTTCACCGGCGAAGAGATCA
AGTACATGCTGTGGCTCTCCGCCGACCTGAAGTTCCGCATCAAGCAGAAGGGA
GAGTACCTCCCGCTGCTGCAAGGGAAGTCGCTGGGGATGATCTTCGAGAAGCG
GTCAACCAGAACCCGGCTGTCAACCGAAACCGGGTTCGCACTGCTGGGGGGAC
ACCCGTGCTTCCTGACCACCCAAGACATCCACCTGGGAGTGAACGAATCGCTGA
CCGACACCGCCCGCGTGCTGAGCTCAATGGCGGACGCCGTGCTGGCCCGCGTGT
ACAAGCAGTCCGACCTGGACACCCTGGCCAAGGAAGCGTCCATCCCGATCATC
AACGGACTGTCCGACCTGTACCACCCGATCCAGATCCTGGCAGACTACCTGACC
CTGCAAGAACACTACAGCTCCCTGAAGGGCCTGACCCTGTCATGGATCGGGGA
CGGGAACAACATCCTGCACTCCATAATGATGTCAGCCGCCAAGTTCGGAATGCA
CCTCCAAGCCGCAACCCCGAAGGGCTACGAACCGGACGCATCAGTGACCAAAC
TGGCCGAGCAGTACGCCAAGGAAAACGGCACCAAGCTCCTGCTGACCAACGAC
CCGCTGGAGGCCGCACACGGGGGGAACGTGCTGATCACCGACACCTGGATCTC
CATGGGACAGGAGGAGGAAAAGAAGAAGCGGCTGCAGGCGTTCCAGGGGTAC
CAGGTCACCATGAAAACCGCGAAGGTCGCGGCATCAGACTGGACCTTCCTGCA
CTGCCTGCCCCGGAAGCCGGAAGAGGTGGACGACGAGGTGTTCTACTCGCCGC
GCTCGCTGGTGTTCCCCGAGGCGGAGAACAGGAAGTGGACCATCATGGCGGTG
ATGGTCAGCCTCCTGACCGACTACTCGCCGCAGCTGCAGAAGCCGAAGTTCTGA
>pARM1883
(SEQ ID NO: 197)
ATGGTGTTCAACCTCCGCATCCTCCTCAACAACGCCGCATTCAGAAACGGGCAC
AACTTCATGGTCAGAAACTTCCGCTGCGGGCAACCCCTACAAAACCGGGTCCAG
CTCAAGGGGCGGGACCTCCTGACCCTGAAGAACTTCACCGGCGAAGAGATCAA
GTACATGCTGTGGCTCTCCGCCGACCTGAAGTTCCGCATCAAGCAGAAGGGAG
AGTACCTCCCGCTGCTGCAAGGGAAGTCGCTGGGGATGATCTTCGAGAAGCGG
TCAACCAGAACCCGGCTGTCAACCGAAACCGGGTTCGCACTGCTGGGGGGACA
CCCGTGCTTCCTGACCACCCAAGACATCCACCTGGGAGTGAACGAATCGCTGAC
CGACACCGCCCGCGTGCTGAGCTCAATGGCGGACGCCGTGCTGGCCCGCGTGTA
CAAGCAGTCCGACCTGGACACCCTGGCCAAGGAAGCGTCCATCCCGATCATCA
ACGGACTGTCCGACCTGTACCACCCGATCCAGATCCTGGCAGACTACCTGACCC
TGCAAGAACACTACAGCTCCCTGAAGGGCCTGACCCTGTCATGGATCGGGGAC
GGGAACAACATCCTGCACTCCATAATGATGTCAGCCGCCAAGTTCGGAATGCAC
CTCCAAGCCGCAACCCCGAAGGGCTACGAACCGGACGCATCAGTGACCAAACT
GGCCGAGCAGTACGCCAAGGAAAACGGCACCAAGCTCCTGCTGACCAACGACC
CGCTGGAGGCCGCACACGGGGGGAACGTGCTGATCACCGACACCTGGATCTCC
ATGGGACAGGAGGAGGAAAAGAAGAAGCGGCTGCAGGCGTTCCAGGGGTACC
AGGTCACCATGAAAACCGCGAAGGTCGCGGCATCAGACTGGACCTTCCTGCAC
TGCCTGCCCCGGAAGCCGGAAGAGGTGGACGACGAGGTGTTCTACTCGCCGCG
CTCGCTGGTGTTCCCCGAGGCGGAGAACAGGAAGTGGACCATCATGGCGGTGA
TGGTCAGCCTCCTGACCGACTACTCGCCGCAGCTGCAGAAGCCGAAGTTCTGA
>pARM1884
(SEQ ID NO: 198)
ATGGTGTTCAACCTCCGCATCCTCCTCAACAACGCCGCATTCAGAAACGGGCAC
AACTTCATGGTCAGAAACTTCCGCTGCGGGCAACCCCTACAAAACCGGGTCCAG
CTCAAGGGGCGGGACCTCCTGACCCTGAAGAACTTCACCGGCGAAGAGATCCG
GTACATGCTGTGGCTCTCCGCCGACCTGAAGTTCCGCATCAAGCAGAAGGGAG
AGTACCTCCCGCTGCTGCAAGGGAAGTCGCTGGGGATGATCTTCGAGAAGCGG
TCAACCAGAACCCGGCTGTCAACCGAAACCGGGTTCGCACTGCTGGGGGGACA
CCCGTGCTTCCTGACCACCCAAGACATCCACCTGGGAGTGAACGAATCGCTGAC
CGACACCGCCCGCGTGCTGAGCTCAATGGCGGACGCCGTGCTGGCCCGCGTGTA
CAAGCAGTCCGACCTGGACACCCTGGCCAAGGAAGCGTCCATCCCGATCATCA
ACGGACTGTCCGACCTGTACCACCCGATCCAGATCCTGGCAGACTACCTGACCC
TGCAAGAACACTACAGCTCCCTGAAGGGCCTGACCCTGTCATGGATCGGGGAC
GGGAACAACATCCTGCACTCCATAATGATGTCAGCCGCCAAGTTCGGAATGCAC
CTCCAAGCCGCAACCCCGAAGGGCTACGAACCGGACGCATCAGTGACCAAACT
GGCCGAGCAGTACGCCAAGGAAAACGGCACCAAGCTCCTGCTGACCAACGACC
CGCTGGAGGCCGCACACGGGGGGAACGTGCTGATCACCGACACCTGGATCTCC
ATGGGACAGGAGGAGGAAAAGAAGAAGCGGCTGCAGGCGTTCCAGGGGTACC
AGGTCACCATGAAAACCGCGAAGGTCGCGGCATCAGACTGGACCTTCCTGCAC
TGCCTGCCCCGGAAGCCGGAAGAGGTGGACGACGAGGTGTTCTACTCGCCGCG
CTCGCTGGTGTTCCCCGAGGCGGAGAACAGGAAGTGGACCATCATGGCGGTGA
TGGTCAGCCTCCTGACCGACTACTCGCCGCAGCTGCAGAAGCCGAAGTTCTGA
>pARM1885
(SEQ ID NO: 199)
ATGCTGGTCAACCTCCGCATCCTCCTCAACAACGCCGCATTCAGAAACGGGCAC
AACTTCATGGTCAGAAACTTCCGCTGCGGGCAACCCCTACAAAACAAGGTCCA
GCTCAAGGGGCGGGACCTCCTGACCCTGAAGAACTTCACCGGCGAAGAGATCA
AGTACATGCTGTGGCTCTCCGCCGACCTGAAGTTCCGCATCAAGCAGAAGGGA
GAGTACCTCCCGCTGCTGCAAGGGAAGTCGCTGGGGATGATCTTCGAGAAGCG
GTCAACCAGAACCCGGCTGTCAACCGAAACCGGGTTCGCACTGCTGGGGGGAC
ACCCGTGCTTCCTGACCACCCAAGACATCCACCTGGGAGTGAACGAATCGCTGA
CCGACACCGCCCGCGTGCTGAGCTCAATGGCGGACGCCGTGCTGGCCCGCGTGT
ACAAGCAGTCCGACCTGGACACCCTGGCCAAGGAAGCGTCCATCCCGATCATC
AACGGACTGTCCGACCTGTACCACCCGATCCAGATCCTGGCAGACTACCTGACC
CTGCAAGAACACTACAGCTCCCTGAAGGGCCTGACCCTGTCATGGATCGGGGA
CGGGAACAACATCCTGCACTCCATAATGATGTCAGCCGCCAAGTTCGGAATGCA
CCTCCAAGCCGCAACCCCGAAGGGCTACGAACCGGACGCATCAGTGACCAAAC
TGGCCGAGCAGTACGCCAAGGAAAACGGCACCAAGCTCCTGCTGACCAACGAC
CCGCTGGAGGCCGCACACGGGGGGAACGTGCTGATCACCGACACCTGGATCTC
CATGGGACAGGAGGAGGAAAAGAAGAAGCGGCTGCAGGCGTTCCAGGGGTAC
CAGGTCACCATGAAAACCGCGAAGGTCGCGGCATCAGACTGGACCTTCCTGCA
CTGCCTGCCCCGGAAGCCGGAAGAGGTGGACGACGAGGTGTTCTACTCGCCGC
GCTCGCTGGTGTTCCCCGAGGCGGAGAACAGGAAGTGGACCATCATGGCGGTG
ATGGTCAGCCTCCTGACCGACTACTCGCCGCAGCTGCAGAAGCCGAAGTTCTGA
>pARM1886
(SEQ ID NO: 200)
ATGCTGGTCAACCTCCGCATCCTCCTCAACAACGCCGCATTCAGAAACGGGCAC
AACTTCATGGTCAGAAACTTCCGCTGCGGGCAACCCCTACAAAACCGGGTCCAG
CTCAAGGGGCGGGACCTCCTGACCCTGAAGAACTTCACCGGCGAAGAGATCAA
GTACATGCTGTGGCTCTCCGCCGACCTGAAGTTCCGCATCAAGCAGAAGGGAG
AGTACCTCCCGCTGCTGCAAGGGAAGTCGCTGGGGATGATCTTCGAGAAGCGG
TCAACCAGAACCCGGCTGTCAACCGAAACCGGGTTCGCACTGCTGGGGGGACA
CCCGTGCTTCCTGACCACCCAAGACATCCACCTGGGAGTGAACGAATCGCTGAC
CGACACCGCCCGCGTGCTGAGCTCAATGGCGGACGCCGTGCTGGCCCGCGTGTA
CAAGCAGTCCGACCTGGACACCCTGGCCAAGGAAGCGTCCATCCCGATCATCA
ACGGACTGTCCGACCTGTACCACCCGATCCAGATCCTGGCAGACTACCTGACCC
TGCAAGAACACTACAGCTCCCTGAAGGGCCTGACCCTGTCATGGATCGGGGAC
GGGAACAACATCCTGCACTCCATAATGATGTCAGCCGCCAAGTTCGGAATGCAC
CTCCAAGCCGCAACCCCGAAGGGCTACGAACCGGACGCATCAGTGACCAAACT
GGCCGAGCAGTACGCCAAGGAAAACGGCACCAAGCTCCTGCTGACCAACGACC
CGCTGGAGGCCGCACACGGGGGGAACGTGCTGATCACCGACACCTGGATCTCC
ATGGGACAGGAGGAGGAAAAGAAGAAGCGGCTGCAGGCGTTCCAGGGGTACC
AGGTCACCATGAAAACCGCGAAGGTCGCGGCATCAGACTGGACCTTCCTGCAC
TGCCTGCCCCGGAAGCCGGAAGAGGTGGACGACGAGGTGTTCTACTCGCCGCG
CTCGCTGGTGTTCCCCGAGGCGGAGAACAGGAAGTGGACCATCATGGCGGTGA
TGGTCAGCCTCCTGACCGACTACTCGCCGCAGCTGCAGAAGCCGAAGTTCTGA
>pARM1887
(SEQ ID NO: 201)
ATGCTGGTCAACCTCCGCATCCTCCTCAACAACGCCGCATTCAGAAACGGGCAC
AACTTCATGGTCAGAAACTTCCGCTGCGGGCAACCCCTACAAAACCGGGTCCAG
CTCAAGGGGCGGGACCTCCTGACCCTGAAGAACTTCACCGGCGAAGAGATCCG
GTACATGCTGTGGCTCTCCGCCGACCTGAAGTTCCGCATCAAGCAGAAGGGAG
AGTACCTCCCGCTGCTGCAAGGGAAGTCGCTGGGGATGATCTTCGAGAAGCGG
TCAACCAGAACCCGGCTGTCAACCGAAACCGGGTTCGCACTGCTGGGGGGACA
CCCGTGCTTCCTGACCACCCAAGACATCCACCTGGGAGTGAACGAATCGCTGAC
CGACACCGCCCGCGTGCTGAGCTCAATGGCGGACGCCGTGCTGGCCCGCGTGTA
CAAGCAGTCCGACCTGGACACCCTGGCCAAGGAAGCGTCCATCCCGATCATCA
ACGGACTGTCCGACCTGTACCACCCGATCCAGATCCTGGCAGACTACCTGACCC
TGCAAGAACACTACAGCTCCCTGAAGGGCCTGACCCTGTCATGGATCGGGGAC
GGGAACAACATCCTGCACTCCATAATGATGTCAGCCGCCAAGTTCGGAATGCAC
CTCCAAGCCGCAACCCCGAAGGGCTACGAACCGGACGCATCAGTGACCAAACT
GGCCGAGCAGTACGCCAAGGAAAACGGCACCAAGCTCCTGCTGACCAACGACC
CGCTGGAGGCCGCACACGGGGGGAACGTGCTGATCACCGACACCTGGATCTCC
ATGGGACAGGAGGAGGAAAAGAAGAAGCGGCTGCAGGCGTTCCAGGGGTACC
AGGTCACCATGAAAACCGCGAAGGTCGCGGCATCAGACTGGACCTTCCTGCAC
TGCCTGCCCCGGAAGCCGGAAGAGGTGGACGACGAGGTGTTCTACTCGCCGCG
CTCGCTGGTGTTCCCCGAGGCGGAGAACAGGAAGTGGACCATCATGGCGGTGA
TGGTCAGCCTCCTGACCGACTACTCGCCGCAGCTGCAGAAGCCGAAGTTCTGA
>pARM1888
(SEQ ID NO: 202)
ATGCTGGTCAACCTGCGCATCCTGCTGAACAACGCCGCCTTCCGCAACGGCCAC
AACTTCATGGTGCGCAACTTCCGCTGCGGCCAGCCCCTGCAGAACAAGGTGCAG
CTGAAGGGCCGCGACCTGCTGACCCTGAAGAACTTCACCGGCGAGGAGATCAA
GTACATGCTGTGGCTGAGCGCCGACCTGAAGTTCCGCATCAAGCAGAAGGGCG
AGTACCTGCCCCTGCTGCAGGGCAAGAGCCTGGGCATGATCTTCGAGAAGCGC
AGCACCCGCACCCGCCTGAGCACCGAGACAGGCTTCGCCCTGCTGGGCGGCCA
CCCCTGCTTCCTGACCACCCAGGACATCCACCTGGGCGTGAACGAGAGCCTGAC
CGACACCGCCCGCGTGCTGAGCAGCATGGCCGACGCCGTGCTGGCCCGCGTGT
ACAAGCAGAGCGACCTGGACACCCTGGCCAAGGAGGCCAGCATCCCCATCATC
AACGGCCTGAGCGACCTGTACCACCCCATCCAGATCCTGGCCGACTACCTGACC
CTGCAGGAGCACTACAGCAGCCTGAAGGGCCTGACCCTGAGCTGGATCGGCGA
CGGCAACAACATCCTGCACAGCATCATGATGAGCGCCGCCAAGTTCGGCATGC
ACCTGCAGGCCGCCACCCCCAAGGGCTACGAGCCCGACGCCAGCGTGACCAAG
CTGGCCGAGCAGTACGCCAAGGAGAACGGCACCAAGCTGCTGCTGACCAACGA
CCCCCTGGAGGCCGCCCACGGCGGCAACGTGCTGATCACCGACACCTGGATCA
GCATGGGCCAGGAGGAGGAGAAGAAGAAGCGCCTGCAGGCCTTCCAGGGCTAC
CAGGTGACCATGAAGACCGCCAAGGTGGCCGCCAGCGACTGGACCTTCCTGCA
CTGCCTGCCCCGCAAGCCCGAGGAGGTGGACGACGAGGTGTTCTACAGCCCCC
GCAGCCTGGTGTTCCCCGAGGCCGAGAACCGCAAGTGGACCATCATGGCCGTG
ATGGTGAGCCTGCTGACCGACTACAGCCCCCAGCTGCAGAAGCCCAAGTTCTGA
>pARM1889
(SEQ ID NO: 203)
ATGCTGGTCAACCTGCGCATCCTGCTGAACAACGCCGCCTTCCGCAACGGCCAC
AACTTCATGGTGCGCAACTTCCGCTGCGGCCAGCCCCTGCAGAACCGGGTGCAG
CTGAAGGGCCGCGACCTGCTGACCCTGAAGAACTTCACCGGCGAGGAGATCAA
GTACATGCTGTGGCTGAGCGCCGACCTGAAGTTCCGCATCAAGCAGAAGGGCG
AGTACCTGCCCCTGCTGCAGGGCAAGAGCCTGGGCATGATCTTCGAGAAGCGC
AGCACCCGCACCCGCCTGAGCACCGAGACAGGCTTCGCCCTGCTGGGCGGCCA
CCCCTGCTTCCTGACCACCCAGGACATCCACCTGGGCGTGAACGAGAGCCTGAC
CGACACCGCCCGCGTGCTGAGCAGCATGGCCGACGCCGTGCTGGCCCGCGTGT
ACAAGCAGAGCGACCTGGACACCCTGGCCAAGGAGGCCAGCATCCCCATCATC
AACGGCCTGAGCGACCTGTACCACCCCATCCAGATCCTGGCCGACTACCTGACC
CTGCAGGAGCACTACAGCAGCCTGAAGGGCCTGACCCTGAGCTGGATCGGCGA
CGGCAACAACATCCTGCACAGCATCATGATGAGCGCCGCCAAGTTCGGCATGC
ACCTGCAGGCCGCCACCCCCAAGGGCTACGAGCCCGACGCCAGCGTGACCAAG
CTGGCCGAGCAGTACGCCAAGGAGAACGGCACCAAGCTGCTGCTGACCAACGA
CCCCCTGGAGGCCGCCCACGGCGGCAACGTGCTGATCACCGACACCTGGATCA
GCATGGGCCAGGAGGAGGAGAAGAAGAAGCGCCTGCAGGCCTTCCAGGGCTAC
CAGGTGACCATGAAGACCGCCAAGGTGGCCGCCAGCGACTGGACCTTCCTGCA
CTGCCTGCCCCGCAAGCCCGAGGAGGTGGACGACGAGGTGTTCTACAGCCCCC
GCAGCCTGGTGTTCCCCGAGGCCGAGAACCGCAAGTGGACCATCATGGCCGTG
ATGGTGAGCCTGCTGACCGACTACAGCCCCCAGCTGCAGAAGCCCAAGTTCTGA
>pARM1890
(SEQ ID NO: 204)
ATGCTTGTCAATCTCCGCATCCTCCTTAACAACGCCGCGTTTAGAAACGGCCAC
AACTTCATGGTCCGGAACTTCAGATGTGGCCAGCCGCTTCAAAACAAGGTCCAG
CTGAAGGGCCGGGATCTTCTGACCCTGAAGAACTTTACTGGCGAAGAGATCAA
GTACATGCTCTGGCTCTCCGCGGACTTGAAGTTCCGCATTAAGCAGAAGGGGGA
ATACCTTCCGCTGCTTCAAGGAAAGAGCCTCGGCATGATCTTTGAGAAGCGCTC
AACCAGGACCCGCCTTTCTACTGAAACTGGGTTCGCGCTGCTCGGTGGCCACCC
CTGCTTCCTGACGACCCAGGACATCCACCTCGGAGTGAACGAATCCCTCACCGA
TACCGCCCGGGTGTTATCGAGCATGGCAGATGCCGTGCTGGCCAGGGTGTACAA
ACAGTCCGATCTGGACACTCTGGCCAAGGAGGCGTCAATTCCTATTATCAACGG
CCTTAGTGACCTCTACCATCCGATTCAGATCCTGGCCGATTACCTCACCCTGCAA
GAACACTACAGCTCCCTGAAGGGTCTGACATTGTCCTGGATCGGCGACGGCAAC
AACATTCTCCATTCCATCATGATGTCCGCCGCAAAATTCGGCATGCATCTTCAA
GCCGCCACGCCGAAGGGTTACGAGCCCGACGCTTCCGTGACTAAGCTCGCCGA
GCAGTACGCTAAGGAGAACGGAACCAAGCTTCTGCTGACTAACGACCCACTAG
AAGCAGCCCACGGGGGCAACGTGCTTATTACTGACACCTGGATCTCCATGGGCC
AGGAAGAAGAGAAAAAGAAGCGGCTGCAGGCGTTCCAGGGATATCAGGTCAC
CATGAAAACCGCCAAGGTCGCTGCCTCCGACTGGACCTTCCTGCACTGCCTGCC
TCGCAAGCCTGAAGAAGTGGACGACGAGGTGTTCTACTCGCCACGGAGCCTCG
TGTTCCCCGAGGCCGAGAATAGAAAGTGGACCATCATGGCCGTGATGGTGTCA
CTGCTCACCGACTACAGCCCGCAGCTTCAGAAGCCCAAGTTCTAG
>pARM1891
(SEQ ID NO: 205)
ATGCTTGTCAATCTCCGCATCCTCCTTAACAACGCCGCGTTTAGAAACGGCCAC
AACTTCATGGTCCGGAACTTCAGATGTGGCCAGCCGCTTCAAAACCGGGTCCAG
CTGAAGGGCCGGGATCTTCTGACCCTGAAGAACTTTACTGGCGAAGAGATCAA
GTACATGCTCTGGCTCTCCGCGGACTTGAAGTTCCGCATTAAGCAGAAGGGGGA
ATACCTTCCGCTGCTTCAAGGAAAGAGCCTCGGCATGATCTTTGAGAAGCGCTC
AACCAGGACCCGCCTTTCTACTGAAACTGGGTTCGCGCTGCTCGGTGGCCACCC
CTGCTTCCTGACGACCCAGGACATCCACCTCGGAGTGAACGAATCCCTCACCGA
TACCGCCCGGGTGTTATCGAGCATGGCAGATGCCGTGCTGGCCAGGGTGTACAA
ACAGTCCGATCTGGACACTCTGGCCAAGGAGGCGTCAATTCCTATTATCAACGG
CCTTAGTGACCTCTACCATCCGATTCAGATCCTGGCCGATTACCTCACCCTGCAA
GAACACTACAGCTCCCTGAAGGGTCTGACATTGTCCTGGATCGGCGACGGCAAC
AACATTCTCCATTCCATCATGATGTCCGCCGCAAAATTCGGCATGCATCTTCAA
GCCGCCACGCCGAAGGGTTACGAGCCCGACGCTTCCGTGACTAAGCTCGCCGA
GCAGTACGCTAAGGAGAACGGAACCAAGCTTCTGCTGACTAACGACCCACTAG
AAGCAGCCCACGGGGGCAACGTGCTTATTACTGACACCTGGATCTCCATGGGCC
AGGAAGAAGAGAAAAAGAAGCGGCTGCAGGCGTTCCAGGGATATCAGGTCAC
CATGAAAACCGCCAAGGTCGCTGCCTCCGACTGGACCTTCCTGCACTGCCTGCC
TCGCAAGCCTGAAGAAGTGGACGACGAGGTGTTCTACTCGCCACGGAGCCTCG
TGTTCCCCGAGGCCGAGAATAGAAAGTGGACCATCATGGCCGTGATGGTGTCA
CTGCTCACCGACTACAGCCCGCAGCTTCAGAAGCCCAAGTTCTAG
>pARM1898
(SEQ ID NO: 206)
ATGGGCCTTGTCAATCTCCGCATCCTCCTTAACAACGCCGCGTTTAGAAACGGC
CACAACTTCATGGTCCGGAACTTCAGATGTGGCCAGCCGCTTCAAAACAAGGTC
CAGCTGAAGGGCCGGGATCTTCTGACCCTGAAGAACTTTACTGGCGAAGAGAT
CAAGTACATGCTCTGGCTCTCCGCGGACTTGAAGTTCCGCATTAAGCAGAAGGG
GGAATACCTTCCGCTGCTTCAAGGAAAGAGCCTCGGCATGATCTTTGAGAAGCG
CTCAACCAGGACCCGCCTTTCTACTGAAACTGGGTTCGCGCTGCTCGGTGGCCA
CCCCTGCTTCCTGACGACCCAGGACATCCACCTCGGAGTGAACGAATCCCTCAC
CGATACCGCCCGGGTGTTATCGAGCATGGCAGATGCCGTGCTGGCCAGGGTGTA
CAAACAGTCCGATCTGGACACTCTGGCCAAGGAGGCGTCAATTCCTATTATCAA
CGGCCTTAGTGACCTCTACCATCCGATTCAGATCCTGGCCGATTACCTCACCCTG
CAAGAACACTACAGCTCCCTGAAGGGTCTGACATTGTCCTGGATCGGCGACGGC
AACAACATTCTCCATTCCATCATGATGTCCGCCGCAAAATTCGGCATGCATCTT
CAAGCCGCCACGCCGAAGGGTTACGAGCCCGACGCTTCCGTGACTAAGCTCGC
CGAGCAGTACGCTAAGGAGAACGGAACCAAGCTTCTGCTGACTAACGACCCAC
TAGAAGCAGCCCACGGGGGCAACGTGCTTATTACTGACACCTGGATCTCCATGG
GCCAGGAAGAAGAGAAAAAGAAGCGGCTGCAGGCGTTCCAGGGATATCAGGT
CACCATGAAAACCGCCAAGGTCGCTGCCTCCGACTGGACCTTCCTGCACTGCCT
GCCTCGCAAGCCTGAAGAAGTGGACGACGAGGTGTTCTACTCGCCACGGAGCC
TCGTGTTCCCCGAGGCCGAGAATAGAAAGTGGACCATCATGGCCGTGATGGTGT
CACTGCTCACCGACTACAGCCCGCAGCTTCAGAAGCCCAAGTTCTAG
>pARM1899
(SEQ ID NO: 207)
ATGGGCCTTGTCAATCTCCGCATCCTCCTTAACAACGCCGCGTTTAGAAACGGC
CACAACTTCATGGTCCGGAACTTCAGATGTGGCCAGCCGCTTCAAAACCGGGTC
CAGCTGAAGGGCCGGGATCTTCTGACCCTGAAGAACTTTACTGGCGAAGAGAT
CAAGTACATGCTCTGGCTCTCCGCGGACTTGAAGTTCCGCATTAAGCAGAAGGG
GGAATACCTTCCGCTGCTTCAAGGAAAGAGCCTCGGCATGATCTTTGAGAAGCG
CTCAACCAGGACCCGCCTTTCTACTGAAACTGGGTTCGCGCTGCTCGGTGGCCA
CCCCTGCTTCCTGACGACCCAGGACATCCACCTCGGAGTGAACGAATCCCTCAC
CGATACCGCCCGGGTGTTATCGAGCATGGCAGATGCCGTGCTGGCCAGGGTGTA
CAAACAGTCCGATCTGGACACTCTGGCCAAGGAGGCGTCAATTCCTATTATCAA
CGGCCTTAGTGACCTCTACCATCCGATTCAGATCCTGGCCGATTACCTCACCCTG
CAAGAACACTACAGCTCCCTGAAGGGTCTGACATTGTCCTGGATCGGCGACGGC
AACAACATTCTCCATTCCATCATGATGTCCGCCGCAAAATTCGGCATGCATCTT
CAAGCCGCCACGCCGAAGGGTTACGAGCCCGACGCTTCCGTGACTAAGCTCGC
CGAGCAGTACGCTAAGGAGAACGGAACCAAGCTTCTGCTGACTAACGACCCAC
TAGAAGCAGCCCACGGGGGCAACGTGCTTATTACTGACACCTGGATCTCCATGG
GCCAGGAAGAAGAGAAAAAGAAGCGGCTGCAGGCGTTCCAGGGATATCAGGT
CACCATGAAAACCGCCAAGGTCGCTGCCTCCGACTGGACCTTCCTGCACTGCCT
GCCTCGCAAGCCTGAAGAAGTGGACGACGAGGTGTTCTACTCGCCACGGAGCC
TCGTGTTCCCCGAGGCCGAGAATAGAAAGTGGACCATCATGGCCGTGATGGTGT
CACTGCTCACCGACTACAGCCCGCAGCTTCAGAAGCCCAAGTTCTAG
>pARM1900
(SEQ ID NO: 208)
ATGGGCGGACTTGTCAATCTCCGCATCCTCCTTAACAACGCCGCGTTTAGAAAC
GGCCACAACTTCATGGTCCGGAACTTCAGATGTGGCCAGCCGCTTCAAAACAAG
GTCCAGCTGAAGGGCCGGGATCTTCTGACCCTGAAGAACTTTACTGGCGAAGA
GATCAAGTACATGCTCTGGCTCTCCGCGGACTTGAAGTTCCGCATTAAGCAGAA
GGGGGAATACCTTCCGCTGCTTCAAGGAAAGAGCCTCGGCATGATCTTTGAGAA
GCGCTCAACCAGGACCCGCCTTTCTACTGAAACTGGGTTCGCGCTGCTCGGTGG
CCACCCCTGCTTCCTGACGACCCAGGACATCCACCTCGGAGTGAACGAATCCCT
CACCGATACCGCCCGGGTGTTATCGAGCATGGCAGATGCCGTGCTGGCCAGGGT
GTACAAACAGTCCGATCTGGACACTCTGGCCAAGGAGGCGTCAATTCCTATTAT
CAACGGCCTTAGTGACCTCTACCATCCGATTCAGATCCTGGCCGATTACCTCAC
CCTGCAAGAACACTACAGCTCCCTGAAGGGTCTGACATTGTCCTGGATCGGCGA
CGGCAACAACATTCTCCATTCCATCATGATGTCCGCCGCAAAATTCGGCATGCA
TCTTCAAGCCGCCACGCCGAAGGGTTACGAGCCCGACGCTTCCGTGACTAAGCT
CGCCGAGCAGTACGCTAAGGAGAACGGAACCAAGCTTCTGCTGACTAACGACC
CACTAGAAGCAGCCCACGGGGGCAACGTGCTTATTACTGACACCTGGATCTCCA
TGGGCCAGGAAGAAGAGAAAAAGAAGCGGCTGCAGGCGTTCCAGGGATATCA
GGTCACCATGAAAACCGCCAAGGTCGCTGCCTCCGACTGGACCTTCCTGCACTG
CCTGCCTCGCAAGCCTGAAGAAGTGGACGACGAGGTGTTCTACTCGCCACGGA
GCCTCGTGTTCCCCGAGGCCGAGAATAGAAAGTGGACCATCATGGCCGTGATG
GTGTCACTGCTCACCGACTACAGCCCGCAGCTTCAGAAGCCCAAGTTCTAG
>pARM1903
(SEQ ID NO: 209)
ATGGCCCTTTTCAATCTCCGCATCCTCCTTAACAACGCCGCGTTTAGAAACGGC
CACAACTTCATGGTCCGGAACTTCAGATGTGGCCAGCCGCTTCAAGGCAAGGTC
CAGCTGAAGGGCCGGGATCTTCTGACCCTGAAGAACTTTACTGGCGAAGAGAT
CAAGTACATGCTCTGGCTCTCCGCGGACTTGAAGTTCCGCATTAAGCAGAAGGG
GGAATACCTTCCGCTGCTTCAAGGAAAGAGCCTCGGCATGATCTTTGAGAAGCG
CTCAACCAGGACCCGCCTTTCTACTGAAACTGGGTTCGCGCTGCTCGGTGGCCA
CCCCTGCTTCCTGACGACCCAGGACATCCACCTCGGAGTGAACGAATCCCTCAC
CGATACCGCCCGGGTGTTATCGAGCATGGCAGATGCCGTGCTGGCCAGGGTGTA
CAAACAGTCCGATCTGGACACTCTGGCCAAGGAGGCGTCAATTCCTATTATCAA
CGGCCTTAGTGACCTCTACCATCCGATTCAGATCCTGGCCGATTACCTCACCCTG
CAAGAACACTACAGCTCCCTGAAGGGTCTGACATTGTCCTGGATCGGCGACGGC
AACAACATTCTCCATTCCATCATGATGTCCGCCGCAAAATTCGGCATGCATCTT
CAAGCCGCCACGCCGAAGGGTTACGAGCCCGACGCTTCCGTGACTAAGCTCGC
CGAGCAGTACGCTAAGGAGAACGGAACCAAGCTTCTGCTGACTAACGACCCAC
TAGAAGCAGCCCACGGGGGCAACGTGCTTATTACTGACACCTGGATCTCCATGG
GCCAGGAAGAAGAGAAAAAGAAGCGGCTGCAGGCGTTCCAGGGATATCAGGT
CACCATGAAAACCGCCAAGGTCGCTGCCTCCGACTGGACCTTCCTGCACTGCCT
GCCTCGCAAGCCTGAAGAAGTGGACGACGAGGTGTTCTACTCGCCACGGAGCC
TCGTGTTCCCCGAGGCCGAGAATAGAAAGTGGACCATCATGGCCGTGATGGTGT
CACTGCTCACCGACTACAGCCCGCAGCTTCAGAAGCCCAAGTTCTAGATAAGTG
AA
>pARM1904
(SEQ ID NO: 210)
ATGGCCCTTTTCAATCTCCGCATCCTCCTTAACAACGCCGCGTTTAGAAACGGC
CACAACTTCATGGTCCGGAACTTCAGATGTGGCCAGCCGCTTCAAGGCCGGGTC
CAGCTGAAGGGCCGGGATCTTCTGACCCTGAAGAACTTTACTGGCGAAGAGAT
CAAGTACATGCTCTGGCTCTCCGCGGACTTGAAGTTCCGCATTAAGCAGAAGGG
GGAATACCTTCCGCTGCTTCAAGGAAAGAGCCTCGGCATGATCTTTGAGAAGCG
CTCAACCAGGACCCGCCTTTCTACTGAAACTGGGTTCGCGCTGCTCGGTGGCCA
CCCCTGCTTCCTGACGACCCAGGACATCCACCTCGGAGTGAACGAATCCCTCAC
CGATACCGCCCGGGTGTTATCGAGCATGGCAGATGCCGTGCTGGCCAGGGTGTA
CAAACAGTCCGATCTGGACACTCTGGCCAAGGAGGCGTCAATTCCTATTATCAA
CGGCCTTAGTGACCTCTACCATCCGATTCAGATCCTGGCCGATTACCTCACCCTG
CAAGAACACTACAGCTCCCTGAAGGGTCTGACATTGTCCTGGATCGGCGACGGC
AACAACATTCTCCATTCCATCATGATGTCCGCCGCAAAATTCGGCATGCATCTT
CAAGCCGCCACGCCGAAGGGTTACGAGCCCGACGCTTCCGTGACTAAGCTCGC
CGAGCAGTACGCTAAGGAGAACGGAACCAAGCTTCTGCTGACTAACGACCCAC
TAGAAGCAGCCCACGGGGGCAACGTGCTTATTACTGACACCTGGATCTCCATGG
GCCAGGAAGAAGAGAAAAAGAAGCGGCTGCAGGCGTTCCAGGGATATCAGGT
CACCATGAAAACCGCCAAGGTCGCTGCCTCCGACTGGACCTTCCTGCACTGCCT
GCCTCGCAAGCCTGAAGAAGTGGACGACGAGGTGTTCTACTCGCCACGGAGCC
TCGTGTTCCCCGAGGCCGAGAATAGAAAGTGGACCATCATGGCCGTGATGGTGT
CACTGCTCACCGACTACAGCCCGCAGCTTCAGAAGCCCAAGTTCTAGATAAGTG
AA
>pARM1905
(SEQ ID NO: 211)
ATGGCCCTTTTCAATCTCCGCATCCTCCTTAACAACGCCGCGTTTAGAAACGGC
CACAACTTCATGGTCCGGAACTTCAGATGTGGCCAGCCGCTTCAAGGCCGGGTC
CAGCTGAAGGGCCGGGATCTTCTGACCCTGAAGAACTTTACTGGCGAAGAGAT
CAGGTACATGCTCTGGCTCTCCGCGGACTTGAAGTTCCGCATTAAGCAGAAGGG
GGAATACCTTCCGCTGCTTCAAGGAAAGAGCCTCGGCATGATCTTTGAGAAGCG
CTCAACCAGGACCCGCCTTTCTACTGAAACTGGGTTCGCGCTGCTCGGTGGCCA
CCCCTGCTTCCTGACGACCCAGGACATCCACCTCGGAGTGAACGAATCCCTCAC
CGATACCGCCCGGGTGTTATCGAGCATGGCAGATGCCGTGCTGGCCAGGGTGTA
CAAACAGTCCGATCTGGACACTCTGGCCAAGGAGGCGTCAATTCCTATTATCAA
CGGCCTTAGTGACCTCTACCATCCGATTCAGATCCTGGCCGATTACCTCACCCTG
CAAGAACACTACAGCTCCCTGAAGGGTCTGACATTGTCCTGGATCGGCGACGGC
AACAACATTCTCCATTCCATCATGATGTCCGCCGCAAAATTCGGCATGCATCTT
CAAGCCGCCACGCCGAAGGGTTACGAGCCCGACGCTTCCGTGACTAAGCTCGC
CGAGCAGTACGCTAAGGAGAACGGAACCAAGCTTCTGCTGACTAACGACCCAC
TAGAAGCAGCCCACGGGGGCAACGTGCTTATTACTGACACCTGGATCTCCATGG
GCCAGGAAGAAGAGAAAAAGAAGCGGCTGCAGGCGTTCCAGGGATATCAGGT
CACCATGAAAACCGCCAAGGTCGCTGCCTCCGACTGGACCTTCCTGCACTGCCT
GCCTCGCAAGCCTGAAGAAGTGGACGACGAGGTGTTCTACTCGCCACGGAGCC
TCGTGTTCCCCGAGGCCGAGAATAGAAAGTGGACCATCATGGCCGTGATGGTGT
CACTGCTCACCGACTACAGCCCGCAGCTTCAGAAGCCCAAGTTCTAGATAAGTG
AA
>pARM1906
(SEQ ID NO: 212)
ATGGCCCTTGTCAATCTCCGCATCCTCCTTAACAACGCCGCGTTTAGAAACGGC
CACAACTTCATGGTCCGGAACTTCAGATGTGGCCAGCCGCTTCAAGGCAGGGTC
CAGCTGAAGGGCCGGGATCTTCTGACCCTGAAGAACTTTACTGGCGAAGAGAT
CAAGTACATGCTCTGGCTCTCCGCGGACTTGAAGTTCCGCATTAAGCAGAAGGG
GGAATACCTTCCGCTGCTTCAAGGAAAGAGCCTCGGCATGATCTTTGAGAAGCG
CTCAACCAGGACCCGCCTTTCTACTGAAACTGGGTTCGCGCTGCTCGGTGGCCA
CCCCTGCTTCCTGACGACCCAGGACATCCACCTCGGAGTGAACGAATCCCTCAC
CGATACCGCCCGGGTGTTATCGAGCATGGCAGATGCCGTGCTGGCCAGGGTGTA
CAAACAGTCCGATCTGGACACTCTGGCCAAGGAGGCGTCAATTCCTATTATCAA
CGGCCTTAGTGACCTCTACCATCCGATTCAGATCCTGGCCGATTACCTCACCCTG
CAAGAACACTACAGCTCCCTGAAGGGTCTGACATTGTCCTGGATCGGCGACGGC
AACAACATTCTCCATTCCATCATGATGTCCGCCGCAAAATTCGGCATGCATCTT
CAAGCCGCCACGCCGAAGGGTTACGAGCCCGACGCTTCCGTGACTAAGCTCGC
CGAGCAGTACGCTAAGGAGAACGGAACCAAGCTTCTGCTGACTAACGACCCAC
TAGAAGCAGCCCACGGGGGCAACGTGCTTATTACTGACACCTGGATCTCCATGG
GCCAGGAAGAAGAGAAAAAGAAGCGGCTGCAGGCGTTCCAGGGATATCAGGT
CACCATGAAAACCGCCAAGGTCGCTGCCTCCGACTGGACCTTCCTGCACTGCCT
GCCTCGCAAGCCTGAAGAAGTGGACGACGAGGTGTTCTACTCGCCACGGAGCC
TCGTGTTCCCCGAGGCCGAGAATAGAAAGTGGACCATCATGGCCGTGATGGTGT
CACTGCTCACCGACTACAGCCCGCAGCTTCAGAAGCCCAAGTTCTAAGTGAATA
GA
>pARM1907
(SEQ ID NO: 213)
ATGGCCCTTTTCAATCTCCGCATCCTCCTTAACAACGCCGCGTTTAGAAACGGC
CACAACTTCATGGTCCGGAACTTCAGATGTGGCCAGCCGCTTCAAGTCAAGGTC
CAGCTGAAGGGCCGGGATCTTCTGACCCTGAAGAACTTTACTGGCGAAGAGAT
CAAGTACATGCTCTGGCTCTCCGCGGACTTGAAGTTCCGCATTAAGCAGAAGGG
GGAATACCTTCCGCTGCTTCAAGGAAAGAGCCTCGGCATGATCTTTGAGAAGCG
CTCAACCAGGACCCGCCTTTCTACTGAAACTGGGTTCGCGCTGCTCGGTGGCCA
CCCCTGCTTCCTGACGACCCAGGACATCCACCTCGGAGTGAACGAATCCCTCAC
CGATACCGCCCGGGTGTTATCGAGCATGGCAGATGCCGTGCTGGCCAGGGTGTA
CAAACAGTCCGATCTGGACACTCTGGCCAAGGAGGCGTCAATTCCTATTATCAA
CGGCCTTAGTGACCTCTACCATCCGATTCAGATCCTGGCCGATTACCTCACCCTG
CAAGAACACTACAGCTCCCTGAAGGGTCTGACATTGTCCTGGATCGGCGACGGC
AACAACATTCTCCATTCCATCATGATGTCCGCCGCAAAATTCGGCATGCATCTT
CAAGCCGCCACGCCGAAGGGTTACGAGCCCGACGCTTCCGTGACTAAGCTCGC
CGAGCAGTACGCTAAGGAGAACGGAACCAAGCTTCTGCTGACTAACGACCCAC
TAGAAGCAGCCCACGGGGGCAACGTGCTTATTACTGACACCTGGATCTCCATGG
GCCAGGAAGAAGAGAAAAAGAAGCGGCTGCAGGCGTTCCAGGGATATCAGGT
CACCATGAAAACCGCCAAGGTCGCTGCCTCCGACTGGACCTTCCTGCACTGCCT
GCCTCGCAAGCCTGAAGAAGTGGACGACGAGGTGTTCTACTCGCCACGGAGCC
TCGTGTTCCCCGAGGCCGAGAATAGAAAGTGGACCATCATGGCCGTGATGGTGT
CACTGCTCACCGACTACAGCCCGCAGCTTCAGAAGCCCAAGTTCTAGATAAGTG
AA
>pARM1908
(SEQ ID NO: 214)
ATGGCCCTTTTCAATCTCCGCATCCTCCTTAACAACGCCGCGTTTAGAAACGGC
CACAACTTCATGGTCCGGAACTTCAGATGTGGCCAGCCGCTTCAAGTCAGGGTC
CAGCTGAAGGGCCGGGATCTTCTGACCCTGAAGAACTTTACTGGCGAAGAGAT
CAAGTACATGCTCTGGCTCTCCGCGGACTTGAAGTTCCGCATTAAGCAGAAGGG
GGAATACCTTCCGCTGCTTCAAGGAAAGAGCCTCGGCATGATCTTTGAGAAGCG
CTCAACCAGGACCCGCCTTTCTACTGAAACTGGGTTCGCGCTGCTCGGTGGCCA
CCCCTGCTTCCTGACGACCCAGGACATCCACCTCGGAGTGAACGAATCCCTCAC
CGATACCGCCCGGGTGTTATCGAGCATGGCAGATGCCGTGCTGGCCAGGGTGTA
CAAACAGTCCGATCTGGACACTCTGGCCAAGGAGGCGTCAATTCCTATTATCAA
CGGCCTTAGTGACCTCTACCATCCGATTCAGATCCTGGCCGATTACCTCACCCTG
CAAGAACACTACAGCTCCCTGAAGGGTCTGACATTGTCCTGGATCGGCGACGGC
AACAACATTCTCCATTCCATCATGATGTCCGCCGCAAAATTCGGCATGCATCTT
CAAGCCGCCACGCCGAAGGGTTACGAGCCCGACGCTTCCGTGACTAAGCTCGC
CGAGCAGTACGCTAAGGAGAACGGAACCAAGCTTCTGCTGACTAACGACCCAC
TAGAAGCAGCCCACGGGGGCAACGTGCTTATTACTGACACCTGGATCTCCATGG
GCCAGGAAGAAGAGAAAAAGAAGCGGCTGCAGGCGTTCCAGGGATATCAGGT
CACCATGAAAACCGCCAAGGTCGCTGCCTCCGACTGGACCTTCCTGCACTGCCT
GCCTCGCAAGCCTGAAGAAGTGGACGACGAGGTGTTCTACTCGCCACGGAGCC
TCGTGTTCCCCGAGGCCGAGAATAGAAAGTGGACCATCATGGCCGTGATGGTGT
CACTGCTCACCGACTACAGCCCGCAGCTTCAGAAGCCCAAGTTCTAGATAAGTG
AA
>pARM1915
(SEQ ID NO: 215)
ATGCTGTTCAACCTGCGCATCCTGCTGAACAACGCCGCCTTCCGCAACGGCCAC
AACTTCATGGTGCGCAACTTCCGCTGCGGCCAGCCCCTGCAGAACAAGGTGCAG
CTGAAGGGCCGCGACCTGCTGACCCTGAAGAACTTCACCGGCGAGGAGATCAA
GTACATGCTGTGGCTGAGCGCCGACCTGAAGTTCCGCATCAAGCAGAAGGGCG
AGTACCTGCCCCTGCTGCAGGGCAAGAGCCTGGGCATGATCTTCGAGAAGCGC
AGCACCCGCACCCGCCTGAGCACCGAGACAGGCTTCGCCCTGCTGGGCGGCCA
CCCCTGCTTCCTGACCACCCAGGACATCCACCTGGGCGTGAACGAGAGCCTGAC
CGACACCGCCCGCGTGCTGAGCAGCATGGCCGACGCCGTGCTGGCCCGCGTGT
ACAAGCAGAGCGACCTGGACACCCTGGCCAAGGAGGCCAGCATCCCCATCATC
AACGGCCTGAGCGACCTGTACCACCCCATCCAGATCCTGGCCGACTACCTGACC
CTGCAGGAGCACTACAGCAGCCTGAAGGGCCTGACCCTGAGCTGGATCGGCGA
CGGCAACAACATCCTGCACAGCATCATGATGAGCGCCGCCAAGTTCGGCATGC
ACCTGCAGGCCGCCACCCCCAAGGGCTACGAGCCCGACGCCAGCGTGACCAAG
CTGGCCGAGCAGTACGCCAAGGAGAACGGCACCAAGCTGCTGCTGACCAACGA
CCCCCTGGAGGCCGCCCACGGCGGCAACGTGCTGATCACCGACACCTGGATCA
GCATGGGCCAGGAGGAGGAGAAGAAGAAGCGCCTGCAGGCCTTCCAGGGCTAC
CAGGTGACCATGAAGACCGCCAAGGTGGCCGCCAGCGACTGGACCTTCCTGCA
CTGCCTGCCCCGCAAGCCCGAGGAGGTGGACGACGAGGTGTTCTACAGCCCCC
GCAGCCTGGTGTTCCCCGAGGCCGAGAACCGCAAGTGGACCATCATGGCCGTG
ATGGTGAGCCTGCTGACCGACTACAGCCCCCAGCTGCAGAAGCCCAAGTTCTGA
>pARM1916
(SEQ ID NO: 216)
ATGGGAGTATTCAACCTGCGCATCCTGCTGAACAACGCCGCCTTCCGCAACGGC
CACAACTTCATGGTGCGCAACTTCCGCTGCGGCCAGCCCCTGCAGAACAAGGTG
CAGCTGAAGGGCCGCGACCTGCTGACCCTGAAGAACTTCACCGGCGAGGAGAT
CAAGTACATGCTGTGGCTGAGCGCCGACCTGAAGTTCCGCATCAAGCAGAAGG
GCGAGTACCTGCCCCTGCTGCAGGGCAAGAGCCTGGGCATGATCTTCGAGAAG
CGCAGCACCCGCACCCGCCTGAGCACCGAGACAGGCTTCGCCCTGCTGGGCGG
CCACCCCTGCTTCCTGACCACCCAGGACATCCACCTGGGCGTGAACGAGAGCCT
GACCGACACCGCCCGCGTGCTGAGCAGCATGGCCGACGCCGTGCTGGCCCGCG
TGTACAAGCAGAGCGACCTGGACACCCTGGCCAAGGAGGCCAGCATCCCCATC
ATCAACGGCCTGAGCGACCTGTACCACCCCATCCAGATCCTGGCCGACTACCTG
ACCCTGCAGGAGCACTACAGCAGCCTGAAGGGCCTGACCCTGAGCTGGATCGG
CGACGGCAACAACATCCTGCACAGCATCATGATGAGCGCCGCCAAGTTCGGCA
TGCACCTGCAGGCCGCCACCCCCAAGGGCTACGAGCCCGACGCCAGCGTGACC
AAGCTGGCCGAGCAGTACGCCAAGGAGAACGGCACCAAGCTGCTGCTGACCAA
CGACCCCCTGGAGGCCGCCCACGGCGGCAACGTGCTGATCACCGACACCTGGA
TCAGCATGGGCCAGGAGGAGGAGAAGAAGAAGCGCCTGCAGGCCTTCCAGGGC
TACCAGGTGACCATGAAGACCGCCAAGGTGGCCGCCAGCGACTGGACCTTCCT
GCACTGCCTGCCCCGCAAGCCCGAGGAGGTGGACGACGAGGTGTTCTACAGCC
CCCGCAGCCTGGTGTTCCCCGAGGCCGAGAACCGCAAGTGGACCATCATGGCC
GTGATGGTGAGCCTGCTGACCGACTACAGCCCCCAGCTGCAGAAGCCCAAGTTC
TGA
>pARM1917
(SEQ ID NO: 217)
ATGGGAGTATTCAACCTGCGCATCCTGCTGAACAACGCCGCCTTCCGCAACGGC
CACAACTTCATGGTGCGCAACTTCCGCTGCGGCCAGCCCCTGCAGAACCGGGTG
CAGCTGAAGGGCCGCGACCTGCTGACCCTGAAGAACTTCACCGGCGAGGAGAT
CCGGTACATGCTGTGGCTGAGCGCCGACCTGAAGTTCCGCATCAAGCAGAAGG
GCGAGTACCTGCCCCTGCTGCAGGGCAAGAGCCTGGGCATGATCTTCGAGAAG
CGCAGCACCCGCACCCGCCTGAGCACCGAGACAGGCTTCGCCCTGCTGGGCGG
CCACCCCTGCTTCCTGACCACCCAGGACATCCACCTGGGCGTGAACGAGAGCCT
GACCGACACCGCCCGCGTGCTGAGCAGCATGGCCGACGCCGTGCTGGCCCGCG
TGTACAAGCAGAGCGACCTGGACACCCTGGCCAAGGAGGCCAGCATCCCCATC
ATCAACGGCCTGAGCGACCTGTACCACCCCATCCAGATCCTGGCCGACTACCTG
ACCCTGCAGGAGCACTACAGCAGCCTGAAGGGCCTGACCCTGAGCTGGATCGG
CGACGGCAACAACATCCTGCACAGCATCATGATGAGCGCCGCCAAGTTCGGCA
TGCACCTGCAGGCCGCCACCCCCAAGGGCTACGAGCCCGACGCCAGCGTGACC
AAGCTGGCCGAGCAGTACGCCAAGGAGAACGGCACCAAGCTGCTGCTGACCAA
CGACCCCCTGGAGGCCGCCCACGGCGGCAACGTGCTGATCACCGACACCTGGA
TCAGCATGGGCCAGGAGGAGGAGAAGAAGAAGCGCCTGCAGGCCTTCCAGGGC
TACCAGGTGACCATGAAGACCGCCAAGGTGGCCGCCAGCGACTGGACCTTCCT
GCACTGCCTGCCCCGCAAGCCCGAGGAGGTGGACGACGAGGTGTTCTACAGCC
CCCGCAGCCTGGTGTTCCCCGAGGCCGAGAACCGCAAGTGGACCATCATGGCC
GTGATGGTGAGCCTGCTGACCGACTACAGCCCCCAGCTGCAGAAGCCCAAGTTC
TGA
>pARM1918
(SEQ ID NO: 218)
ATGCTGGTATTCAACCTGCGCATCCTGCTGAACAACGCCGCCTTCCGCAACGGC
CACAACTTCATGGTGCGCAACTTCCGCTGCGGCCAGCCCCTGCAGAACCGGGTG
CAGCTGAAGGGCCGCGACCTGCTGACCCTGAAGAACTTCACCGGCGAGGAGAT
CCGGTACATGCTGTGGCTGAGCGCCGACCTGAAGTTCCGCATCAAGCAGAAGG
GCGAGTACCTGCCCCTGCTGCAGGGCAAGAGCCTGGGCATGATCTTCGAGAAG
CGCAGCACCCGCACCCGCCTGAGCACCGAGACAGGCTTCGCCCTGCTGGGCGG
CCACCCCTGCTTCCTGACCACCCAGGACATCCACCTGGGCGTGAACGAGAGCCT
GACCGACACCGCCCGCGTGCTGAGCAGCATGGCCGACGCCGTGCTGGCCCGCG
TGTACAAGCAGAGCGACCTGGACACCCTGGCCAAGGAGGCCAGCATCCCCATC
ATCAACGGCCTGAGCGACCTGTACCACCCCATCCAGATCCTGGCCGACTACCTG
ACCCTGCAGGAGCACTACAGCAGCCTGAAGGGCCTGACCCTGAGCTGGATCGG
CGACGGCAACAACATCCTGCACAGCATCATGATGAGCGCCGCCAAGTTCGGCA
TGCACCTGCAGGCCGCCACCCCCAAGGGCTACGAGCCCGACGCCAGCGTGACC
AAGCTGGCCGAGCAGTACGCCAAGGAGAACGGCACCAAGCTGCTGCTGACCAA
CGACCCCCTGGAGGCCGCCCACGGCGGCAACGTGCTGATCACCGACACCTGGA
TCAGCATGGGCCAGGAGGAGGAGAAGAAGAAGCGCCTGCAGGCCTTCCAGGGC
TACCAGGTGACCATGAAGACCGCCAAGGTGGCCGCCAGCGACTGGACCTTCCT
GCACTGCCTGCCCCGCAAGCCCGAGGAGGTGGACGACGAGGTGTTCTACAGCC
CCCGCAGCCTGGTGTTCCCCGAGGCCGAGAACCGCAAGTGGACCATCATGGCC
GTGATGGTGAGCCTGCTGACCGACTACAGCCCCCAGCTGCAGAAGCCCAAGTTC
TGA
>pARM1919
(SEQ ID NO: 219)
ATGGGAGTATTCAACCTGCGCATCCTGCTGAACAACGCCGCCTTCCGCAACGGC
CACAACTTCATGGTGCGCAACTTCCGCTGCGGCCAGCCCCTGCAGAACAAGGTG
CAGCTGAAGGGCCGCGACCTGCTGACCCTGAAGAACTTCACCGGCGAGGAGAT
CAAGTACATGCTGTGGCTGAGCGCCGACCTGAAGTTCCGCATCAAGCAGAAGG
GCGAGTACCTGCCCCTGCTGCAGGGCAAGAGCCTGGGCATGATCTTCGAGAAG
CGCAGCACCCGCACCCGCCTGAGCACCGAGACAGGCTTCGCCCTGCTGGGCGG
CCACCCCTGCTTCCTGACCACCCAGGACATCCACCTGGGCGTGAACGAGAGCCT
GACCGACACCGCCCGCGTGCTGAGCAGCATGGCCGACGCCGTGCTGGCCCGCG
TGTACAAGCAGAGCGACCTGGACACCCTGGCCAAGGAGGCCAGCATCCCCATC
ATCAACGGCCTGAGCGACCTGTACCACCCCATCCAGATCCTGGCCGACTACCTG
ACCCTGCAGGAGCACTACAGCAGCCTGAAGGGCCTGACCCTGAGCTGGATCGG
CGACGGCAACAACATCCTGCACAGCATCATGATGAGCGCCGCCAAGTTCGGCA
TGCACCTGCAGGCCGCCACCCCCAAGGGCTACGAGCCCGACGCCAGCGTGACC
AAGCTGGCCGAGCAGTACGCCAAGGAGAACGGCACCAAGCTGCTGCTGACCAA
CGACCCCCTGGAGGCCGCCCACGGCGGCAACGTGCTGATCACCGACACCTGGA
TCAGCATGGGCCAGGAGGAGGAGAAGAAGAAGCGCCTGCAGGCCTTCCAGGGC
TACCAGGTGACCATGAAGACCGCCAAGGTGGCCGCCAGCGACTGGACCTTCCT
GCACTGCCTGCCCCGCAAGCCCGAGGAGGTGGACGACGAGGTGTTCTACAGCC
CCCGCAGCCTGGTGTTCCCCGAGGCCGAGAACCGCAAGTGGACCATCATGGCC
GTGATGGTGAGCCTGCTGACCGACTACAGCCCCCAGCTGCAGAAGCCCAAGTTC
TGA
>pARM1920
(SEQ ID NO: 220)
ATGGGAGTATTCAACCTGCGCATCCTGCTGAACAACGCCGCCTTCCGCAACGGC
CACAACTTCATGGTGCGCAACTTCCGCTGCGGCCAGCCCCTGCAGAACCGGGTG
CAGCTGAAGGGCCGCGACCTGCTGACCCTGAAGAACTTCACCGGCGAGGAGAT
CCGGTACATGCTGTGGCTGAGCGCCGACCTGAAGTTCCGCATCAAGCAGAAGG
GCGAGTACCTGCCCCTGCTGCAGGGCAAGAGCCTGGGCATGATCTTCGAGAAG
CGCAGCACCCGCACCCGCCTGAGCACCGAGACAGGCTTCGCCCTGCTGGGCGG
CCACCCCTGCTTCCTGACCACCCAGGACATCCACCTGGGCGTGAACGAGAGCCT
GACCGACACCGCCCGCGTGCTGAGCAGCATGGCCGACGCCGTGCTGGCCCGCG
TGTACAAGCAGAGCGACCTGGACACCCTGGCCAAGGAGGCCAGCATCCCCATC
ATCAACGGCCTGAGCGACCTGTACCACCCCATCCAGATCCTGGCCGACTACCTG
ACCCTGCAGGAGCACTACAGCAGCCTGAAGGGCCTGACCCTGAGCTGGATCGG
CGACGGCAACAACATCCTGCACAGCATCATGATGAGCGCCGCCAAGTTCGGCA
TGCACCTGCAGGCCGCCACCCCCAAGGGCTACGAGCCCGACGCCAGCGTGACC
AAGCTGGCCGAGCAGTACGCCAAGGAGAACGGCACCAAGCTGCTGCTGACCAA
CGACCCCCTGGAGGCCGCCCACGGCGGCAACGTGCTGATCACCGACACCTGGA
TCAGCATGGGCCAGGAGGAGGAGAAGAAGAAGCGCCTGCAGGCCTTCCAGGGC
TACCAGGTGACCATGAAGACCGCCAAGGTGGCCGCCAGCGACTGGACCTTCCT
GCACTGCCTGCCCCGCAAGCCCGAGGAGGTGGACGACGAGGTGTTCTACAGCC
CCCGCAGCCTGGTGTTCCCCGAGGCCGAGAACCGCAAGTGGACCATCATGGCC
GTGATGGTGAGCCTGCTGACCGACTACAGCCCCCAGCTGCAGAAGCCCAAGTTC
TGA
>pARM1921
(SEQ ID NO: 221)
ATGCTGGTATTCAACCTGCGCATCCTGCTGAACAACGCCGCCTTCCGCAACGGC
CACAACTTCATGGTGCGCAACTTCCGCTGCGGCCAGCCCCTGCAGAACCGGGTG
CAGCTGAAGGGCCGCGACCTGCTGACCCTGAAGAACTTCACCGGCGAGGAGAT
CCGGTACATGCTGTGGCTGAGCGCCGACCTGAAGTTCCGCATCAAGCAGAAGG
GCGAGTACCTGCCCCTGCTGCAGGGCAAGAGCCTGGGCATGATCTTCGAGAAG
CGCAGCACCCGCACCCGCCTGAGCACCGAGACAGGCTTCGCCCTGCTGGGCGG
CCACCCCTGCTTCCTGACCACCCAGGACATCCACCTGGGCGTGAACGAGAGCCT
GACCGACACCGCCCGCGTGCTGAGCAGCATGGCCGACGCCGTGCTGGCCCGCG
TGTACAAGCAGAGCGACCTGGACACCCTGGCCAAGGAGGCCAGCATCCCCATC
ATCAACGGCCTGAGCGACCTGTACCACCCCATCCAGATCCTGGCCGACTACCTG
ACCCTGCAGGAGCACTACAGCAGCCTGAAGGGCCTGACCCTGAGCTGGATCGG
CGACGGCAACAACATCCTGCACAGCATCATGATGAGCGCCGCCAAGTTCGGCA
TGCACCTGCAGGCCGCCACCCCCAAGGGCTACGAGCCCGACGCCAGCGTGACC
AAGCTGGCCGAGCAGTACGCCAAGGAGAACGGCACCAAGCTGCTGCTGACCAA
CGACCCCCTGGAGGCCGCCCACGGCGGCAACGTGCTGATCACCGACACCTGGA
TCAGCATGGGCCAGGAGGAGGAGAAGAAGAAGCGCCTGCAGGCCTTCCAGGGC
TACCAGGTGACCATGAAGACCGCCAAGGTGGCCGCCAGCGACTGGACCTTCCT
GCACTGCCTGCCCCGCAAGCCCGAGGAGGTGGACGACGAGGTGTTCTACAGCC
CCCGCAGCCTGGTGTTCCCCGAGGCCGAGAACCGCAAGTGGACCATCATGGCC
GTGATGGTGAGCCTGCTGACCGACTACAGCCCCCAGCTGCAGAAGCCCAAGTTC
TGA
>pARM1925
(SEQ ID NO: 222)
ATGTTGTTCAACTTGAGGATCTTGTTGAACAACGCCGCCTTCAGGAACGGACAC
AACTTCATGGTAAGGAACTTCAGGTGCGGACAGCCCTTGCAGAACAAAGTACA
GTTGAAAGGAAGGGACTTGTTGACATTGAAAAACTTCACAGGAGAAGAAATCA
AATACATGTTGTGGTTGTCGGCCGACTTGAAATTCAGGATCAAACAGAAAGGA
GAATACTTGCCCTTGTTGCAGGGAAAATCGTTGGGAATGATCTTCGAAAAAAGG
TCGACAAGGACAAGGTTGTCGACAGAAACAGGATTCGCCTTGTTGGGAGGACA
CCCCTGCTTCTTGACAACACAGGACATCCACTTGGGAGTAAACGAATCGTTGAC
AGACACAGCCAGGGTATTGTCGTCGATGGCCGACGCCGTATTGGCCAGGGTAT
ACAAACAGTCGGACTTGGACACATTGGCCAAAGAAGCCTCGATCCCCATCATC
AACGGATTGTCGGACTTGTACCACCCCATCCAGATCTTGGCCGACTACTTGACA
TTGCAGGAACACTACTCGTCGTTGAAAGGATTGACATTGTCGTGGATCGGAGAC
GGAAACAACATCTTGCACTCGATCATGATGTCGGCCGCCAAATTCGGAATGCAC
TTGCAGGCCGCCACACCCAAAGGATACGAACCCGACGCCTCGGTAACAAAATT
GGCCGAACAGTACGCCAAAGAAAACGGAACAAAATTGTTGTTGACAAACGACC
CCTTGGAAGCCGCCCACGGAGGAAACGTATTGATCACAGACACATGGATCTCG
ATGGGACAGGAAGAAGAAAAAAAAAAAAGGTTGCAGGCCTTCCAGGGATACC
AGGTAACAATGAAAACAGCCAAAGTAGCCGCCTCGGACTGGACATTCTTGCAC
TGCTTGCCCAGGAAACCCGAAGAAGTAGACGACGAAGTATTCTACTCGCCCAG
GTCGTTGGTATTCCCCGAAGCCGAAAACAGGAAATGGACAATCATGGCCGTAA
TGGTATCGTTGTTGACAGACTACTCGCCCCAGTTGCAGAAACCCAAATTCTGAA
TAGTGAA
>pARM1926
(SEQ ID NO: 223)
ATGCTGTTCAACCTGCGCATCCTGCTGAACAACGCCGCCTTCCGCAACGGCCAC
AACTTCATGGTGCGCAACTTCCGCTGCGGCCAGCCCCTGCAGGGCAAGGTGCAG
CTGAAGGGCCGCGACCTGCTGACCCTGAAGAACTTCACCGGCGAGGAGATCAA
GTACATGCTGTGGCTGAGCGCCGACCTGAAGTTCCGCATCAAGCAGAAGGGCG
AGTACCTGCCCCTGCTGCAGGGCAAGAGCCTGGGCATGATCTTCGAGAAGCGC
AGCACCCGCACCCGCCTGAGCACCGAGACAGGCTTCGCCCTGCTGGGCGGCCA
CCCCTGCTTCCTGACCACCCAGGACATCCACCTGGGCGTGAACGAGAGCCTGAC
CGACACCGCCCGCGTGCTGAGCAGCATGGCCGACGCCGTGCTGGCCCGCGTGT
ACAAGCAGAGCGACCTGGACACCCTGGCCAAGGAGGCCAGCATCCCCATCATC
AACGGCCTGAGCGACCTGTACCACCCCATCCAGATCCTGGCCGACTACCTGACC
CTGCAGGAGCACTACAGCAGCCTGAAGGGCCTGACCCTGAGCTGGATCGGCGA
CGGCAACAACATCCTGCACAGCATCATGATGAGCGCCGCCAAGTTCGGCATGC
ACCTGCAGGCCGCCACCCCCAAGGGCTACGAGCCCGACGCCAGCGTGACCAAG
CTGGCCGAGCAGTACGCCAAGGAGAACGGCACCAAGCTGCTGCTGACCAACGA
CCCCCTGGAGGCCGCCCACGGCGGCAACGTGCTGATCACCGACACCTGGATCA
GCATGGGCCAGGAGGAGGAGAAGAAGAAGCGCCTGCAGGCCTTCCAGGGCTAC
CAGGTGACCATGAAGACCGCCAAGGTGGCCGCCAGCGACTGGACCTTCCTGCA
CTGCCTGCCCCGCAAGCCCGAGGAGGTGGACGACGAGGTGTTCTACAGCCCCC
GCAGCCTGGTGTTCCCCGAGGCCGAGAACCGCAAGTGGACCATCATGGCCGTG
ATGGTGAGCCTGCTGACCGACTACAGCCCCCAGCTGCAGAAGCCCAAGTTCTGA
>pARM1927
(SEQ ID NO: 224)
ATGCTGTTCAACCTGCGCATCCTGCTGAACAACGCCGCCTTCCGCAACGGCCAC
AACTTCATGGTGCGCAACTTCCGCTGCGGCCAGCCCCTGCAGGGCCGGGTGCAG
CTGAAGGGCCGCGACCTGCTGACCCTGAAGAACTTCACCGGCGAGGAGATCAA
GTACATGCTGTGGCTGAGCGCCGACCTGAAGTTCCGCATCAAGCAGAAGGGCG
AGTACCTGCCCCTGCTGCAGGGCAAGAGCCTGGGCATGATCTTCGAGAAGCGC
AGCACCCGCACCCGCCTGAGCACCGAGACAGGCTTCGCCCTGCTGGGCGGCCA
CCCCTGCTTCCTGACCACCCAGGACATCCACCTGGGCGTGAACGAGAGCCTGAC
CGACACCGCCCGCGTGCTGAGCAGCATGGCCGACGCCGTGCTGGCCCGCGTGT
ACAAGCAGAGCGACCTGGACACCCTGGCCAAGGAGGCCAGCATCCCCATCATC
AACGGCCTGAGCGACCTGTACCACCCCATCCAGATCCTGGCCGACTACCTGACC
CTGCAGGAGCACTACAGCAGCCTGAAGGGCCTGACCCTGAGCTGGATCGGCGA
CGGCAACAACATCCTGCACAGCATCATGATGAGCGCCGCCAAGTTCGGCATGC
ACCTGCAGGCCGCCACCCCCAAGGGCTACGAGCCCGACGCCAGCGTGACCAAG
CTGGCCGAGCAGTACGCCAAGGAGAACGGCACCAAGCTGCTGCTGACCAACGA
CCCCCTGGAGGCCGCCCACGGCGGCAACGTGCTGATCACCGACACCTGGATCA
GCATGGGCCAGGAGGAGGAGAAGAAGAAGCGCCTGCAGGCCTTCCAGGGCTAC
CAGGTGACCATGAAGACCGCCAAGGTGGCCGCCAGCGACTGGACCTTCCTGCA
CTGCCTGCCCCGCAAGCCCGAGGAGGTGGACGACGAGGTGTTCTACAGCCCCC
GCAGCCTGGTGTTCCCCGAGGCCGAGAACCGCAAGTGGACCATCATGGCCGTG
ATGGTGAGCCTGCTGACCGACTACAGCCCCCAGCTGCAGAAGCCCAAGTTCTGA
>pARM1928
(SEQ ID NO: 225)
ATGCTGTTCAACCTGCGCATCCTGCTGAACAACGCCGCCTTCCGCAACGGCCAC
AACTTCATGGTGCGCAACTTCCGCTGCGGCCAGCCCCTGCAGGGCCGGGTGCAG
CTGAAGGGCCGCGACCTGCTGACCCTGAAGAACTTCACCGGCGAGGAGATCCG
GTACATGCTGTGGCTGAGCGCCGACCTGAAGTTCCGCATCAAGCAGAAGGGCG
AGTACCTGCCCCTGCTGCAGGGCAAGAGCCTGGGCATGATCTTCGAGAAGCGC
AGCACCCGCACCCGCCTGAGCACCGAGACAGGCTTCGCCCTGCTGGGCGGCCA
CCCCTGCTTCCTGACCACCCAGGACATCCACCTGGGCGTGAACGAGAGCCTGAC
CGACACCGCCCGCGTGCTGAGCAGCATGGCCGACGCCGTGCTGGCCCGCGTGT
ACAAGCAGAGCGACCTGGACACCCTGGCCAAGGAGGCCAGCATCCCCATCATC
AACGGCCTGAGCGACCTGTACCACCCCATCCAGATCCTGGCCGACTACCTGACC
CTGCAGGAGCACTACAGCAGCCTGAAGGGCCTGACCCTGAGCTGGATCGGCGA
CGGCAACAACATCCTGCACAGCATCATGATGAGCGCCGCCAAGTTCGGCATGC
ACCTGCAGGCCGCCACCCCCAAGGGCTACGAGCCCGACGCCAGCGTGACCAAG
CTGGCCGAGCAGTACGCCAAGGAGAACGGCACCAAGCTGCTGCTGACCAACGA
CCCCCTGGAGGCCGCCCACGGCGGCAACGTGCTGATCACCGACACCTGGATCA
GCATGGGCCAGGAGGAGGAGAAGAAGAAGCGCCTGCAGGCCTTCCAGGGCTAC
CAGGTGACCATGAAGACCGCCAAGGTGGCCGCCAGCGACTGGACCTTCCTGCA
CTGCCTGCCCCGCAAGCCCGAGGAGGTGGACGACGAGGTGTTCTACAGCCCCC
GCAGCCTGGTGTTCCCCGAGGCCGAGAACCGCAAGTGGACCATCATGGCCGTG
ATGGTGAGCCTGCTGACCGACTACAGCCCCCAGCTGCAGAAGCCCAAGTTCTGA
>pARM1929
(SEQ ID NO: 226)
ATGCTGTTCAACCTGCGCATCCTGCTGAACAACGCCGCCTTCCGCAACGGCCAC
AACTTCATGGTGCGCAACTTCCGCTGCGGCCAGCCCCTGCAGGGCAAGGTGCAG
CTGAAGGGCCGCGACCTGCTGACCCTGAAGAACTTCACCGGCGAGGAGATCAA
GTACATGCTGTGGCTGAGCGCCGACCTGAAGTTCCGCATCAAGCAGAAGGGCG
AGTACCTGCCCCTGCTGCAGGGCAAGAGCCTGGGCATGATCTTCGAGAAGCGC
AGCACCCGCACCCGCCTGAGCACCGAGACAGGCTTCGCCCTGCTGGGCGGCCA
CCCCTGCTTCCTGACCACCCAGGACATCCACCTGGGCGTGAACGAGAGCCTGAC
CGACACCGCCCGCGTGCTGAGCAGCATGGCCGACGCCGTGCTGGCCCGCGTGT
ACAAGCAGAGCGACCTGGACACCCTGGCCAAGGAGGCCAGCATCCCCATCATC
AACGGCCTGAGCGACCTGTACCACCCCATCCAGATCCTGGCCGACTACCTGACC
CTGCAGGAGCACTACAGCAGCCTGAAGGGCCTGACCCTGAGCTGGATCGGCGA
CGGCAACAACATCCTGCACAGCATCATGATGAGCGCCGCCAAGTTCGGCATGC
ACCTGCAGGCCGCCACCCCCAAGGGCTACGAGCCCGACGCCAGCGTGACCAAG
CTGGCCGAGCAGTACGCCAAGGAGAACGGCACCAAGCTGCTGCTGACCAACGA
CCCCCTGGAGGCCGCCCACGGCGGCAACGTGCTGATCACCGACACCTGGATCA
GCATGGGCCAGGAGGAGGAGAAGAAGAAGCGCCTGCAGGCCTTCCAGGGCTAC
CAGGTGACCATGAAGACCGCCAAGGTGGCCGCCAGCGACTGGACCTTCCTGCA
CTGCCTGCCCCGCAAGCCCGAGGAGGTGGACGACGAGGTGTTCTACAGCCCCC
GCAGCCTGGTGTTCCCCGAGGCCGAGAACCGCAAGTGGACCATCATGGCCGTG
ATGGTGAGCCTGCTGACCGACTACAGCCCCCAGCTGCAGAAGCCCAAGTTCTGA
>pARM2016
(SEQ ID NO: 227)
ATGCTGTTCAACCTGCGCATCCTGCTGAACAACGCCGCCTTCCGCAACGGCCAC
AACTTCATGGTGCGCAACTTCCGCTGCGGCCAGCCCCTGCAGAACAAGGTGCAG
CTGAAGGGCCGCGACCTGCTGACCCTGAAGAACTTCACCGGCGAGGAGATCAA
GTACATGCTGTGGCTGAGCGCCGACCTGAAGTTCCGCATCAAGCAGAAGGGCG
AGTACCTGCCCCTGCTGCAGGGCAAGAGCCTGGGCATGATCTTCGAGAAGCGC
AGCACCCGCACCCGCCTGAGCACCGAGACAGGCTTCGCCCTGCTGGGCGGCCA
CCCCTGCTTCCTGACCACCCAGGACATCCACCTGGGCGTGAACGAGAGCCTGAC
CGACACCGCCCGCGTGCTGAGCAGCATGGCCGACGCCGTGCTGGCCCGCGTGT
ACAAGCAGAGCGACCTGGACACCCTGGCCAAGGAGGCCAGCATCCCCATCATC
AACGGCCTGAGCGACCTGTACCACCCCATCCAGATCCTGGCCGACTACCTGACC
CTGCAGGAGCACTACAGCAGCCTGAAGGGCCTGACCCTGAGCTGGATCGGCGA
CGGCAACAACATCCTGCACAGCATCATGATGAGCGCCGCCAAGTTCGGCATGC
ACCTGCAGGCCGCCACCCCCAAGGGCTACGAGCCCGACGCCAGCGTGACCAAG
CTGGCCGAGCAGTACGCCAAGGAGAACGGCACCAAGCTGCTGCTGACCAACGA
CCCCCTGGAGGCCGCCCACGGCGGCAACGTGCTGATCACCGACACCTGGATCA
GCATGGGCCAGGAGGAGGAGAAGAAGAAGCGCCTGCAGGCCTTCCAGGGCTAC
CAGGTGACCATGAAGACCGCCAAGGTGGCCGCCAGCGACTGGACCTTCCTGCA
CTGCCTGCCCCGCAAGCCCGAGGAGGTGGACGACGAGGTGTTCTACAGCCCCC
GCAGCCTGGTGTTCCCCGAGGCCGAGAACCGCAAGTGGACCATCATGGCCGTG
ATGGTGAGCCTGCTGACCGACTACAGCCCCCAGCTGCAGAAGCCCAAGTTCTGA
>pARM2260
(SEQ ID NO: 228)
ATGCTGGTATTCAACCTGCGCATCCTGCTGAACAACGCCGCCTTCCGCAACGGC
CACAACTTCATGGTGCGCAACTTCCGCTGCGGCCAGCCCCTGCAGAACCGGGTG
CAGCTGAAGGGCCGCGACCTGCTGACCCTGAAGAACTTCACCGGCGAGGAGAT
CCGGTACATGCTGTGGCTGAGCGCCGACCTGAAGTTCCGCATCAAGCAGAAGG
GCGAGTACCTGCCCCTGCTGCAGGGCAAGAGCCTGGGCATGATCTTCGAGAAG
CGCAGCACCCGCACCCGCCTGAGCACCGAGACAGGCTTCGCCCTGCTGGGCGG
CCACCCCTGCTTCCTGACCACCCAGGACATCCACCTGGGCGTGAACGAGAGCCT
GACCGACACCGCCCGCGTGCTGAGCAGCATGGCCGACGCCGTGCTGGCCCGCG
TGTACAAGCAGAGCGACCTGGACACCCTGGCCAAGGAGGCCAGCATCCCCATC
ATCAACGGCCTGAGCGACCTGTACCACCCCATCCAGATCCTGGCCGACTACCTG
ACCCTGCAGGAGCACTACAGCAGCCTGAAGGGCCTGACCCTGAGCTGGATCGG
CGACGGCAACAACATCCTGCACAGCATCATGATGAGCGCCGCCAAGTTCGGCA
TGCACCTGCAGGCCGCCACCCCCAAGGGCTACGAGCCCGACGCCAGCGTGACC
AAGCTGGCCGAGCAGTACGCCAAGGAGAACGGCACCAAGCTGCTGCTGACCAA
CGACCCCCTGGAGGCCGCCCACGGCGGCAACGTGCTGATCACCGACACCTGGA
TCAGCATGGGCCAGGAGGAGGAGAAGAAGAAGCGCCTGCAGGCCTTCCAGGGC
TACCAGGTGACCATGAAGACCGCCAAGGTGGCCGCCAGCGACTGGACCTTCCT
GCACTGCCTGCCCCGCAAGCCCGAGGAGGTGGACGACGAGGTGTTCTACAGCC
CCCGCAGCCTGGTGTTCCCCGAGGCCGAGAACCGCAAGTGGACCATCATGGCC
GTGATGGTGAGCCTGCTGACCGACTACAGCCCCCAGCTGCAGAAGCCCAAGTTC
TGA
>pARM2262
(SEQ ID NO: 229)
ATGCTGGTATTCAACCTGCGCATCCTGCTGAACAACGCCGCCTTCCGCAACGGC
CACAACTTCATGGTGCGCAACTTCCGCTGCGGCCAGCCCCTGCAGAACCGGGTG
CAGCTGAAGGGCCGCGACCTGCTGACCCTGAAGAACTTCACCGGCGAGGAGAT
CCGGTACATGCTGTGGCTGAGCGCCGACCTGAAGTTCCGCATCAAGCAGAAGG
GCGAGTACCTGCCCCTGCTGCAGGGCAAGAGCCTGGGCATGATCTTCGAGAAG
CGCAGCACCCGCACCCGCCTGAGCACCGAGACAGGCTTCGCCCTGCTGGGCGG
CCACCCCTGCTTCCTGACCACCCAGGACATCCACCTGGGCGTGAACGAGAGCCT
GACCGACACCGCCCGCGTGCTGAGCAGCATGGCCGACGCCGTGCTGGCCCGCG
TGTACAAGCAGAGCGACCTGGACACCCTGGCCAAGGAGGCCAGCATCCCCATC
ATCAACGGCCTGAGCGACCTGTACCACCCCATCCAGATCCTGGCCGACTACCTG
ACCCTGCAGGAGCACTACAGCAGCCTGAAGGGCCTGACCCTGAGCTGGATCGG
CGACGGCAACAACATCCTGCACAGCATCATGATGAGCGCCGCCAAGTTCGGCA
TGCACCTGCAGGCCGCCACCCCCAAGGGCTACGAGCCCGACGCCAGCGTGACC
AAGCTGGCCGAGCAGTACGCCAAGGAGAACGGCACCAAGCTGCTGCTGACCAA
CGACCCCCTGGAGGCCGCCCACGGCGGCAACGTGCTGATCACCGACACCTGGA
TCAGCATGGGCCAGGAGGAGGAGAAGAAGAAGCGCCTGCAGGCCTTCCAGGGC
TACCAGGTGACCATGAAGACCGCCAAGGTGGCCGCCAGCGACTGGACCTTCCT
GCACTGCCTGCCCCGCAAGCCCGAGGAGGTGGACGACGAGGTGTTCTACAGCC
CCCGCAGCCTGGTGTTCCCCGAGGCCGAGAACCGCAAGTGGACCATCATGGCC
GTGATGGTGAGCCTGCTGACCGACTACAGCCCCCAGCTGCAGAAGCCCAAGTTC
TGA

| | | |
|---|---|---|
| AT5G61250 | AACCAAUCGAAAGAAACCAAA | SEQ ID NO: 230 |
| AT5G46430 | CUCUAAUCACCAGGAGUAAAA | SEQ ID NO: 231 |
| AT5G47110 | GAGAGAGAUCUUAACAAAAAA | SEQ ID NO: 232 |
| AT1G03110 | UGUGUAACAACAACAACAACA | SEQ ID NO: 233 |
| AT3G12380 | CCGCAGUAGGAAGAGAAAGCC | SEQ ID NO: 234 |
| AT5G45910 | AAAAAAAAAAGAAAUCAUAAA | SEQ ID NO: 235 |
| AT1G07260 | GAGAGAAGAAAGAAGAAGACG | SEQ ID NO: 236 |
| AT3G55500 | CAAUUAAAAAUACUUACCAAA | SEQ ID NO: 237 |
| AT3G46230 | GCAAACAGAGUAAGCGAAACG | SEQ ID NO: 238 |
| AT2G36170 | GCGAAGAAGACGAACGCAAAG | SEQ ID NO: 239 |
| AT1G10660 | UUAGGACUGUAUUGACUGGCC | SEQ ID NO: 240 |
| AT4G14340 | AUCAUCGGAAUUCGGAAAAAG | SEQ ID NO: 241 |
| AT1G49310 | AAAACAAAAGUUAAAGCAGAC | SEQ ID NO: 242 |
| AT4G14360 | UUUAUCUCAAAUAAGAAGGCA | SEQ ID NO: 243 |
| AT1G28520 | GGUGGGGAGGUGAGAUUUCUU | SEQ ID NO: 244 |
| AT1G20160 | UGAUUAGGAAACUACAAAGCC | SEQ ID NO: 245 |
| AT5G37370 | CAUUUUUCAAUUUCAUAAAAC | SEQ ID NO: 246 |
| AT4G11320 | UUACUUUUAAGCCCAACAAAA | SEQ ID NO: 247 |
| AT5G40850 | GGCGUGUGUGUGUGUUGUUGA | SEQ ID NO: 248 |
| AT1G06150 | GUGGUGAAGGGGAAGGUUUAG | SEQ ID NO: 249 |
| AT2G26080 | UUGUUUUUUUUUGGUUUGGUU | SEQ ID NO: 250 |

mARM2260
(SEQ ID NO: 251)
AGGAUUAUUACAUCAAAACAAAAAGCCGCCACCAUGCUGGUAUUCAACCUGC
GCAUCCUGCUGAACAACGCCGCCUUCCGCAACGGCCACAACUUCAUGGUGCG
CAACUUCCGCUGCGGCCAGCCCCUGCAGAACCGGGUGCAGCUGAAGGGCCGC
GACCUGCUGACCCUGAAGAACUUCACCGGCGAGGAGAUCCGGUACAUGCUGU
GGCUGAGCGCCGACCUGAAGUUCCGCAUCAAGCAGAAGGGCGAGUACCUGCC
CCUGCUGCAGGGCAAGAGCCUGGGCAUGAUCUUCGAGAAGCGCAGCACCCGC
ACCCGCCUGAGCACCGAGACAGGCUUCGCCCUGCUGGGCGGCCACCCCUGCUU
CCUGACCACCCAGGACAUCCACCUGGGCGUGAACGAGAGCCUGACCGACACC
GCCCGCGUGCUGAGCAGCAUGGCCGACGCCGUGCUGGCCCGCGUGUACAAGC
AGAGCGACCUGGACACCCUGGCCAAGGAGGCCAGCAUCCCCAUCAUCAACGG
CCUGAGCGACCUGUACCACCCCAUCCAGAUCCUGGCCGACUACCUGACCCUGC
AGGAGCACUACAGCAGCCUGAAGGGCCUGACCCUGAGCUGGAUCGGCGACGG
CAACAACAUCCUGCACAGCAUCAUGAUGAGCGCCGCCAAGUUCGGCAUGCAC
CUGCAGGCCGCCACCCCCAAGGGCUACGAGCCCGACGCCAGCGUGACCAAGCU
GGCCGAGCAGUACGCCAAGGAGAACGGCACCAAGCUGCUGCUGACCAACGAC
CCCCUGGAGGCCGCCCACGGCGGCAACGUGCUGAUCACCGACACCUGGAUCA
GCAUGGGCCAGGAGGAGGAGAAGAAGAAGCGCCUGCAGGCCUUCCAGGGCUA
CCAGGUGACCAUGAAGACCGCCAAGGUGGCCGCCAGCGACUGGACCUUCCUG
CACUGCCUGCCCCGCAAGCCCGAGGAGGUGGACGACGAGGUGUUCUACAGCC
CCCGCAGCCUGGUGUUCCCCGAGGCCGAGAACCGCAAGUGGACCAUCAUGGC
CGUGAUGGUGAGCCUGCUGACCGACUACAGCCCCCAGCUGCAGAAGCCCAAG
UUCUGAGGUCUCUAGUAAUGAGCUGGAGCCUCGGUAGCCGUUCCUCCUGCCC
GCUGGGCCUCCCAACGGGCCCUCCUCCCCUCCUUGCACCGGCCCUUCCUGGUC
UUUGAAUAAAGUCUGAGUGGGCAGCAUCUAGAAAAAAAAAAAAAAAAAAAA
AAAAAAAAAAAAAAAAAAAAAAAAAAAAAAAAAAAAAAAAAAAAAAAAAAA
AAAAAAAAAAAAAAAAAAAAAAAAAAAAA
mARM2262
(SEQ ID NO: 252)
AGGAUUAUUACAUCAAAACAAAAAGCCGCCACCAUGCUGGUAUUCAACCUGC
GCAUCCUGCUGAACAACGCCGCCUUCCGCAACGGCCACAACUUCAUGGUGCG
CAACUUCCGCUGCGGCCAGCCCCUGCAGAACCGGGUGCAGCUGAAGGGCCGC
GACCUGCUGACCCUGAAGAACUUCACCGGCGAGGAGAUCCGGUACAUGCUGU
GGCUGAGCGCCGACCUGAAGUUCCGCAUCAAGCAGAAGGGCGAGUACCUGCC
CCUGCUGCAGGGCAAGAGCCUGGGCAUGAUCUUCGAGAAGCGCAGCACCCGC
ACCCGCCUGAGCACCGAGACAGGCUUCGCCCUGCUGGGCGGCCACCCCUGCUU
CCUGACCACCCAGGACAUCCACCUGGGCGUGAACGAGAGCCUGACCGACACC
GCCCGCGUGCUGAGCAGCAUGGCCGACGCCGUGCUGGCCCGCGUGUACAAGC
AGAGCGACCUGGACACCCUGGCCAAGGAGGCCAGCAUCCCCAUCAUCAACGG
CCUGAGCGACCUGUACCACCCCAUCCAGAUCCUGGCCGACUACCUGACCCUGC
AGGAGCACUACAGCAGCCUGAAGGGCCUGACCCUGAGCUGGAUCGGCGACGG
CAACAACAUCCUGCACAGCAUCAUGAUGAGCGCCGCCAAGUUCGGCAUGCAC
CUGCAGGCCGCCACCCCCAAGGGCUACGAGCCCGACGCCAGCGUGACCAAGCU
GGCCGAGCAGUACGCCAAGGAGAACGGCACCAAGCUGCUGCUGACCAACGAC
CCCCUGGAGGCCGCCCACGGCGGCAACGUGCUGAUCACCGACACCUGGAUCA
GCAUGGGCCAGGAGGAGGAGAAGAAGAAGCGCCUGCAGGCCUUCCAGGGCUA
CCAGGUGACCAUGAAGACCGCCAAGGUGGCCGCCAGCGACUGGACCUUCCUG
CACUGCCUGCCCCGCAAGCCCGAGGAGGUGGACGACGAGGUGUUCUACAGCC
CCCGCAGCCUGGUGUUCCCCGAGGCCGAGAACCGCAAGUGGACCAUCAUGGC
CGUGAUGGUGAGCCUGCUGACCGACUACAGCCCCCAGCUGCAGAAGCCCAAG
UUCUGAGGUCUCUAGUAAUGAGCUGGAGCCUCGGUAGCCGUUCCUCCUGCCC
GCUGGGCCUCCCAACGGGCCCUCCUCCCCUCCUUGCACCGGCCCUUCCUGGUC
UUUGAAUAAAGUCUGAGUGGGCAGCAUCUAGAAAAAAAAAAAAAAAAAAAA
AAAAAAAAAAAAAAAAAAAAAAAAAAAAAAAAAAAAAAAAAAAAAAAAAAA
AAAAAAAAAAAAAAAAAAAAAAAAAAAAAGCU
mARM2016
(SEQ ID NO: 253)
AGGAUUAUUACAUCAAAACAAAAAGCCGCCACCAUGCUGUUCAACCUGCGCA
UCCUGCUGAACAACGCCGCCUUCCGCAACGGCCACAACUUCAUGGUGCGCAA
CUUCCGCUGCGGCCAGCCCCUGCAGAACAAGGUGCAGCUGAAGGGCCGCGAC
CUGCUGACCCUGAAGAACUUCACCGGCGAGGAGAUCAAGUACAUGCUGUGGC
UGAGCGCCGACCUGAAGUUCCGCAUCAAGCAGAAGGGCGAGUACCUGCCCCU
GCUGCAGGGCAAGAGCCUGGGCAUGAUCUUCGAGAAGCGCAGCACCCGCACC
CGCCUGAGCACCGAGACAGGCUUCGCCCUGCUGGGCGGCCACCCCUGCUUCCU
GACCACCCAGGACAUCCACCUGGGCGUGAACGAGAGCCUGACCGACACCGCC
CGCGUGCUGAGCAGCAUGGCCGACGCCGUGCUGGCCCGCGUGUACAAGCAGA
GCGACCUGGACACCCUGGCCAAGGAGGCCAGCAUCCCCAUCAUCAACGGCCU
GAGCGACCUGUACCACCCCAUCCAGAUCCUGGCCGACUACCUGACCCUGCAG
GAGCACUACAGCAGCCUGAAGGGCCUGACCCUGAGCUGGAUCGGCGACGGCA
ACAACAUCCUGCACAGCAUCAUGAUGAGCGCCGCCAAGUUCGGCAUGCACCU
GCAGGCCGCCACCCCCAAGGGCUACGAGCCCGACGCCAGCGUGACCAAGCUG
GCCGAGCAGUACGCCAAGGAGAACGGCACCAAGCUGCUGCUGACCAACGACC
CCCUGGAGGCCGCCCACGGCGGCAACGUGCUGAUCACCGACACCUGGAUCAG
CAUGGGCCAGGAGGAGGAGAAGAAGAAGCGCCUGCAGGCCUUCCAGGGCUAC
CAGGUGACCAUGAAGACCGCCAAGGUGGCCGCCAGCGACUGGACCUUCCUGC
ACUGCCUGCCCCGCAAGCCCGAGGAGGUGGACGACGAGGUGUUCUACAGCCC
CCGCAGCCUGGUGUUCCCCGAGGCCGAGAACCGCAAGUGGACCAUCAUGGCC
GUGAUGGUGAGCCUGCUGACCGACUACAGCCCCCAGCUGCAGAAGCCCAAGU
UCUGAGGUCUCUAGUAAUGAGCUGGAGCCUCGGUAGCCGUUCCUCCUGCCCG
CUGGGCCUCCCAACGGGCCCUCCUCCCCUCCUUGCACCGGCCCUUCCUGGUCU
UUGAAUAAAGUCUGAGUGGGCAGCAUCUAGAAAAAAAAAAAAAAAAAAAAA
AAAAAAAAAAAAAAAAAAAAAAAAAAAAAAAAAAAAAAAAAAAAAAAAAAA
AAAAAAAAAAAAAAAAAAAAAAAAAAAA

1. A modified protein having an amino acid sequence derived from an amino acid sequence of a human wild-type protein, wherein
(i) the amino-acid sequence of the human wild-type protein has been modified to remove one or more ubiquitination sites identified as being present in the amino acid sequence of the human wild-type protein but not being present in a homologous non-human animal wild-type protein;
(ii) the human wild-type protein has a signal peptide located at a terminal portion, and wherein the amino acid sequence of the signal peptide has been modified by changing an amino acid at the +1 or +2 position or by adding de novo an amino acid at the +1 or +2 position;
(iii) the human wild-type protein has a signal peptide located at a terminal portion, and wherein the amino-acid sequence of the human wild-type protein has been modified to remove one or more ubiquitination sites identified as being present in the amino acid sequence of the human wild-type protein but not being present in a homologous non-human animal wild-type protein; or
(iv) both (ii) and (iii).

2. The modified protein of claim 1, wherein the homologous non-human animal wild-type protein is a mouse protein.

3. (canceled)

4. (canceled)

5. The modified protein of claim 1, wherein the one or more ubiquitination sites are removed by changing an amino acid at the ubiquitination site.

6. (canceled)

7. The modified protein of claim 1, wherein the amino acid residue at the one or more ubiquitination sites is changed to an arginine residue.

8. (canceled)

9. The modified protein of claim 1, wherein the signal peptide is located at the N-terminus.

10. The modified protein of claim 1, wherein the signal peptide directs translocation of the wild-type protein
(a) for extracellular excretion;
(b) to an organelle selected from the group consisting of a mitochondrion, a nucleus, a lysosome, a peroxisome, an endoplasmic reticulum, a Golgi apparatus, and a plasma membrane; or
(c) to an interior portion of an intracellular organelle, wherein the intracellular organelle is a mitochondrion.

11. (canceled)

12. (canceled)

13. (canceled)

14. (canceled)

15. The modified protein of claim 1, wherein the amino acid sequence of the signal peptide has been modified
(a) by changing an amino acid at the +1 or +2 position, wherein the amino acid at the +1 or +2 position is changed to a stabilizing amino acid selected from the group consisting of alanine, glycine, methionine, serine, threonine, valine, and proline; or
(b) by adding de novo an amino acid at the +1 or +2 position, wherein the amino acid added at the +1 or +2 position is a stabilizing amino acid selected from the group consisting of alanine, glycine, methionine, serine, threonine, valine, and proline.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. The modified protein of claim 1, wherein the human wild-type protein is selected from the group consisting of OTC, ASL, PAH, ABCB4, ABCB11, PAH, AGL, CFTR, MUT, PCCA, PCCB, ASS1, FAH, HMBS, ATP7B, PFIC2, LDLR, G6PC, AGXT, FXN, PAL, BCKDHA, BCKDHB, DBT, UGT1A1, SLC25A13, CD46, CFH, CFI, FIX, FVII, FVIII, C2, C3, C5, GCHD, CBS, MPI, LNL, SERPINGI, UROC1, SMPD1, GLA, GAA, GRHPR, ATP8B1, SERPINCI, PROS1, GBA, ACADVL, HFE, BCKDA, CDG1B, SERPINA1, BMPR2, ENG, ACVR1, SMAD4, BMPR9, HBB, FLCN, HSP1, AP3B1, HPS3, HPS4, HPS5, HPS6, DTNBP1, BLOC1S3, PLDN, AP3D1, BRAF, NF-1, SLC34A2, FBN1, COL1A1, COL1A2, COL1A3, COL5A1, COL5A2, ADAMTS2, PLOD1, TNXB, ABCA3, SP-B, SP-C, GBA, NPC1, NPC2, FOXF1, NKX2-1, SFTPB, SFTPC, ABCA3, CSF2RA, SFTPD, MUC5B, BMPR2, EIF2AK4, CSF2RB, DNAH5, DNAI1, DNAH11, AKR1D1, AMACR, ATP8B1, CYP7A1, FOXC2, GATA2, GHR, HSD3B7, IGFALS, IKBKG, JAGI, KIF11, NOTCHI, NOTCH2, NR1H4, SOX18, TJP2, P53, P73, P63, VIPAS39, VPS33B, EPO, ARGI, CPS1, NAGS, NOS, KRAS, OX40L, IL12, VEGF-A, MMA, TTR, PCSK9, AT, and ALAS1.

36. A polynucleotide comprising a sequence encoding the modified protein of claim 1.

37. (canceled)

38. (canceled)

39. (canceled)

40. The polynucleotide of claim 36, wherein the polynucleotide is an mRNA comprising an open reading frame encoding the modified protein, wherein the open reading frame
(a) is a codon-optimized open reading frame; or
(b) is optimized to have a theoretical minimum of uridines possible.

41. (canceled)

42. (canceled)

43. The polynucleotide of claim 36, wherein the polynucleotide is an mRNA comprising one or more chemically-modified nucleotides selected from the group consisting of 5-hydroxycytidine, 5-methylcytidine, 5-hydroxymethylcytidine, 5-carboxycytidine, 5-formylcytidine, 5-methoxycytidine, 5-propynylcytidine, 2-thiocytidine, 5-hydroxyuridine, 5-methyluridine, 5,6-dihydro-5-methyluridine, 2′-O-methyluridine, 2′-O-methyl-5-methyluridine, 2′-fluoro-2′-deoxyuridine, 2′-amino-2′-deoxyuridine, 2′-azido-2′-deoxyuridine, 4-thiouridine, 5-hydroxymethyluridine, 5-carboxyuridine, 5-carboxymethylesteruridine, 5-formyluridine, 5-methoxyuridine, 5-propynyluridine, 5-bromouridine, 5-iodouridine, 5-fluorouridine, pseudouridine, 2′-O-methyl-pseudouridine, N¹-hydroxypseudouridine, N¹-methylpseudouridine, 2′-O-methyl-N¹-methylpseudouridine, N¹-ethylpseudouridine, N¹-hydroxymethylpseudouridine, Arauridine, N⁶-methyladenosine, 2-aminoadenosine, 3-methyladenosine, 7-deazaadenosine, 8-oxoadenosine, inosine, thienoguanosine, 7-deazaguanosine, 8-oxoguanosine, and 6-O-methylguanine.

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. A composition comprising one or more polynucleotides of claim 36 and a pharmaceutically acceptable carrier, wherein the one or more polynucleotides are mRNAs, and wherein the carrier comprises a transfection reagent, a nanoparticle, or a liposome.

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. A method of modifying a protein of interest, wherein the protein of interest is a human wild-type protein, the method comprising the steps of:
i) identifying ubiquitination sites in the amino acid sequence of the human wild-type protein which are not present in the amino acid sequence of a homologous non-human animal wild-type protein; and
ii) removing at least one of the ubiquitination sites identified in step (i) from the amino acid sequence of the human wild-type protein to provide a modified protein of interest.

69. (canceled)

70. (canceled)

71. The method of claim 68, wherein the homologous non-human animal wild-type protein is a murine protein.

72. The method of claim 68, wherein the removing of step (ii) comprises
(a) replacing an amino acid residue at the at least one of the ubiquitination sites; or
(b) replacing a lysine residue at the at least one of the ubiquitination sites with an arginine residue.

73. (canceled)

74. The method of claim 68, wherein the human wild-type protein comprises a signal peptide at a terminal portion, and the method further comprises modifying the amino acid sequence of the signal peptide by changing an amino acid at the +1 or +2 position or by adding an amino acid at the +1 or +2 position.

75. The method of claim 74, wherein the signal peptide is located at the N-terminus.

76. The method of claim 74, wherein the signal peptide directs translocation of the human wild-type protein
(a) for extracellular excretion;
(b) to an organelle selected from the group consisting of a mitochondrion, a nucleus, a lysosome, a peroxisome, an endoplasmic reticulum, a Golgi apparatus, and a plasma membrane; or
(c) to an interior portion of an organelle, wherein the organelle is a mitochondrion.

77. (canceled)

78. (canceled)

79. (canceled)

80. (canceled)

81. The method of claim 74, wherein modifying the amino acid sequence of the signal peptide comprises
(a) changing an amino acid at the +1 or +2 position, wherein the amino acid at the +1 or +2 position is changed to a stabilizing amino acid selected from the group consisting of alanine, glycine, methionine, serine, threonine, valine, and proline; or
(b) adding an amino acid at the +1 or +2 position, wherein the amino acid added at the +1 or +2 position is a stabilizing amino acid selected from the group consisting of alanine, glycine, methionine, serine, threonine, valine, and proline.

82. (canceled)

83. (canceled)

84. (canceled)

85. The method of claim 74, wherein the human wild-type protein is selected from the group consisting of OTC, ASL, PAH, ABCB4, ABCB11, PAH, AGL, CFTR, MUT, PCCA, PCCB, ASS1, FAH, HMBS, ATP7B, PFIC2, LDLR, G6PC, AGXT, FXN, PAL, BCKDHA, BCKDHB, DBT, UGT1A1, SLC25A13, CD46, CFH, CFI, FIX, FVII, FVIII, C2, C3, C5, GCHD, CBS, MPI, LNL, SERPINGI, UROC1, SMPD1, GLA, GAA, GRHPR, ATP8B1, SERPINCI, PROS1, GBA, ACADVL, HFE, BCKDA, CDG1B, SERPINA1, BMPR2, ENG, ACVR1, SMAD4, BMPR9, HBB, FLCN, HSP1, AP3B1, HPS3, HPS4, HPS5, HPS6, DTNBP1, BLOC1S3, PLDN, AP3D1, BRAF, NF-1, SLC34A2, FBN1, COL1A1, COL1A2, COL1A3, COL5A1, COL5A2, ADAMTS2, PLOD1, TNXB, ABCA3, SP-B, SP-C, GBA, NPC1, NPC2, FOXF1, NKX2-1, SFTPB, SFTPC, ABCA3, CSF2RA, SFTPD, MUC5B, BMPR2, EIF2AK4, CSF2RB, DNAH5, DNAI1, DNAH11, AKR1D1, AMACR, ATP8B1, CYP7A1, FOXC2, GATA2, GHR, HSD3B7, IGFALS, IKBKG, JAG1, KIF11, NOTCH1, NOTCH2, NR1H4, SOX18, TJP2, P53, P73, P63, VIPAS39, VPS33B, EPO, ARGI, CPS1, NAGS, NOS, KRAS, OX40L, IL12, VEGF-A, MMA, TTR, PCSK9, AT, and ALAS1.

86. (canceled)

87. (canceled)
